(19) United States
(12) Patent Application Publication
(10) Pub. No.: US 2018/0251431 A1
(43) Pub. Date: Sep. 6, 2018

(54) 1,3-SUBSTITUTED PYRAZOLE COMPOUNDS USEFUL FOR REDUCTION OF VERY LONG CHAIN FATTY ACID LEVELS

(71) Applicant: VERTEX PHARMACEUTICALS INCORPORATED, Boston, MA (US)

(72) Inventors: Paul S. Charifson, Framingham, MA (US); Jon H. Come, Cambridge, MA (US); John J. Court, Littleton, MA (US); Zachary Gale-Day, Brookline, MA (US); Wenxin Gu, Concord, MA (US); Katrina L. Jackson, Weston, MA (US); Sanjay Shivayogi Magavi, Cambridge, MA (US); Suganthini S. Nanthakumar, Newton, MA (US); Steven Michael Ronkin, Watertown, MA (US); Rebecca Jane Swett, Somerville, MA (US); Qing Tang, Boxborough, MA (US)

(21) Appl. No.: 15/836,406

(22) Filed: Dec. 8, 2017

Related U.S. Application Data
(60) Provisional application No. 62/432,449, filed on Dec. 9, 2016.

(51) Int. Cl. C07D 231/40; C07D 401/04; C07D 409/12; C07D 403/04; C07D 401/12; C07D 417/04; C07D 405/04; C07D 405/14; C07D 401/14; C07D 403/14; C07D 409/14; C07D 413/14; C07D 417/14; C07D 417/12; C07D 413/04

(52) U.S. Cl. CPC C07D 231/40; C07D 413/04; C07D 409/12; C07D 403/04; C07D 401/12; C07D 417/04; C07D 405/04; C07D 405/14; C07D 401/14; C07D 403/14; C07D 409/14; C07D 413/14; C07D 417/14; C07D 417/12; C07D 401/04

(57) ABSTRACT

Disclosed are chemical entities which are compounds of Formula (I) and pharmaceutically acceptable salts thereof, wherein Formula (I) has the structure,

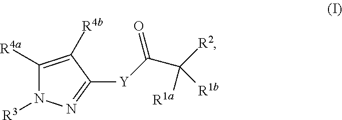

R^{1a}, R^{1b}, R², R³, R^{4a}, R^{4b} and Y are as defined herein. These chemical entities are useful for reduction of very long chain fatty acid levels. These chemical entities and pharmaceutically acceptable compositions comprising such chemical entities can be useful for treating various diseases, disorders and conditions, such as adrenoleukodystrophy (ALD).

### BACKGROUND

Adrenoleukodystrophy (ALD) (also known as X-linked adrenoleukodystrophy or X-adrenoleukodystrophy (X-ALD)) patients suffer from debilitating, and often fatal, neurological effects and adrenal insufficiency often associated with one or more mutations in the ATP binding cassette transporter D1 (ABCD1) gene. ABCD1 plays a critical role in very long chain fatty acid (VLCFA) degradation and, as such, ALD patients typically have elevated VLCFA levels that are thought to be causative of the pathology in ALD. The prevalence of ALD is 1 in 20,000 to 50,000 individuals worldwide. The overall incidence of ALD is estimated to be 1 in 17,000 newborns (males and females). In males there are two predominant phenotypes: cerebral ALD (CALD) and adrenomyeloneuropathy (AMN). CALD is the more extreme form, which presents with rapidly progressive inflammatory demyelination of the brain, leading to rapid cognitive and neurological decline. If untreated, CALD patients die within approximately 2 years of symptom onset. Over the course of their lifetime, approximately 60% of males with ALD will develop CALD, most frequently between the ages of about 3 and about 12 (35 to 40%), with continued (albeit decreasing) risk during adulthood. Adult males with ALD will develop adrenomyeloneuropathy (AMN), a slowly progressive axonopathy with first symptoms appearing around 20 to 30 years of age. AMN is characterized by chronic myelopathy with progressive spastic paraparesis, sensory ataxia, sphincter dysfunction and impotence, commonly associated with primary adrenocortical and/or testicular insufficiency. Approximately 7,000 to 10,000 males in the US and EU combined will develop AMN. Women with ALD are also affected and not merely carriers: >80% of these individuals develop signs and symptoms of myelopathy by the age of 60 years. Approximately 12,000 to 15,000 women in the US and EU combined will eventually develop AMN. Female ABCD1 heterozygotes exhibit approximately half the plasma VLCFA elevation observed in males, never develop the cerebral form of the disease, and develop more modest, but debilitating, AMN-like symptoms later in life. Therefore, about a 50% to about a 75% reduction in VLCFA levels relative to a patient's baseline VLCFA level may be sufficient to prevent cerebral ALD, delay onset, and/or reduce disease severity and progression.

Mutations in any of three separate genes in the VLCFA degradation pathway have been associated with VLCFA accumulation and demyelinating diseases in humans. In addition to mutations in ABCD1, mutations in Acyl-CoA oxidase (ACOX1) or D-Bifunctional protein (DBP) also are associated with accumulation of VLCFA and demyelinating disorders, supporting the hypothesis that increased VLCFA cause the underlying pathophysiology of ALD.

### SUMMARY

There are few treatment options available for ALD patients and their families. One treatment for CALD is an allogenic hematopoietic stem cell transplant (HSCT), but this is effective only if the disease is identified early and a match can be found. Allogenic HSCT is a high-risk procedure, with significant mortality associated with the ablation procedure and graft versus host disease. HSCT is currently used for children affected with CALD; limited data is available regarding effectiveness in adults with CALD, and it has no effect on the subsequent development of AMN in adults. Another treatment for ALD, though not approved for such, has been Lorenzo's oil (LO). Research has suggested that LO has not been able to correct accumulation of VLCFA in brains of ALD patients (Rasmussen et al., Neurochem. Res. (1994) 19(8):1073-82; Poulos et al., Ann Neurol. (1994) 36(5):741-6). Accordingly, there is a need for the development of therapeutic agents useful in the treatment of ALD (for example, CALD, AMN, or both) or other disorders associated with deficiency in very long-chain fatty acids (VLCFA) degradation, associated with deficiency in VLCFA transport into the peroxisomes, associated with accumulation of very long-chain fatty acids (VLCFA), or associated with a benefit from a treatment that lowers VLCFA levels. Deficiency of ABCD1 protein (also known as ALD protein) can lead to transport defects of VLCFA into the peroxisome due to, for example, loss of protein expression or the protein being misfunctional or non-functional. Deficiency of Acyl-CoA Binding Domain Containing 5 (ACBD5), Acyl-CoA oxidase (ACOX1), or D-Bifunctional protein can lead to defects in VLCFA degradation within the peroxisome due to, for example, loss of protein expression or the protein being misfunctional or non-functional.

The chemical entities provided herein can reduce VLCFA levels (also referred to herein as VLCFA concentration) and can be useful for treating (including reducing symptoms of, preventing the onset of, or both) ALD and other diseases, disorders, or conditions associated with accumulation of VLCFA, associated with impaired peroxisomal function (e.g., impaired transport of VLCFA into the peroxisomes or impaired degradation/metabolism of VLCFA (e.g., impaired peroxisomal oxidation within peroxisomes)), or associated with a benefit from a treatment that lowers VLCFA levels. In some embodiments, the chemical entities provided herein can enter the central nervous system (CNS) (e.g., brain, spinal cord, or both). Therefore, in some embodiments, the chemical entities can reduce VLCFA levels in the CNS. In some embodiments, the chemical entities provided herein can reversibly reduce VLCFA levels. Reversibly reducing VLCFA means that the VLCFA levels are reduced when a cell or subject is treated with a chemical entity herein and, when treatment with a chemical entity has been stopped or discontinued, the VLCFA levels return back to about the VLCFA baseline levels prior to treatment. Thus, in some aspects the present invention relates to chemical entities (i.e., free compounds represented by a structure of Formula (I), such as free compounds of Formula (II), (III), (A), (B), (C), (1), (3), (II.A), (II.B), (II.C), (II.1), (III.A), (III.B), (III.C), (III.1), (A.1), (B.1), (C.1), (II.A.1), (II.B.1), (II.C.1), (III.A.1), (III.A.1a), (III.A.1b), (III.A.3), (III.B.1) and/or (III.C.1), including compounds described herein such as those in Table 1, and pharmaceutically acceptable salts thereof) useful for reduction of VLCFA levels. The chemical entities can be useful for treating ALD and other diseases, disorders, or conditions described above and herein. The present invention also relates to pharmaceutically acceptable compositions comprising the chemical entities described herein; methods of reduction of VLCFA levels (e.g., in a cell; in a subject) using the chemical entities described herein; methods of treating of various diseases, disorders, and conditions using the chemical entities described herein; chemical entities for use in a method of reduction of VLCFA levels or treating of various diseases, disorders, and conditions described herein; use of the chemical entities described herein or pharmaceutical composition comprising the chemical entities described herein in the manufacture of a medicament for reduction of VLCFA levels or for treating various diseases, disorders, and conditions described herein; processes for preparing the chemical entities described herein; intermediates useful in the preparation of the chemical entities described herein; and methods of using the chemical entities in in vitro applications.

In some aspects, the present invention provides a chemical entity (a “provided chemical entity”) which is a free compound of Formula (I) or a pharmaceutically acceptable salt thereof, wherein Formula (I) has the structure,

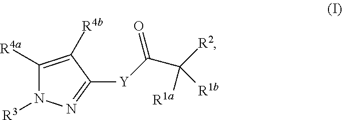

each of R^{1a} and R^{1b} independently is —H, C₁₋₄ alkyl, C₁₋₄ haloalkyl, —(C(R^{J1a}₂))₁₋₂—OH, —(C(R^{J1a}₂))₁₋₂—OR^{J1}, —(C(R^{J1a}₂))₁₋₂—SR^{J1}, —(C(R^{J1a}₂))₁₋₂—NH₂, —(C(R^{J1a}₂))₁₋₂—NHR^{J1}, —(C(R^{J1a}₂))₁₋₂—NR^{J1}₂, C₃₋₆ cycloalkyl or a 3- to 6-membered monocyclic heterocycle containing 1 ring heteroatom selected from O, N, and S,
   wherein the 3- to 6-membered monocyclic heterocycle does not contain a heteroatom bonded to the carbon to which R^{1a} and R^{1b} are attached,
   wherein each instance of R^{J1} is independently C₁₋₃ alkyl or C₁₋₄ haloalkyl,
   wherein each instance of R^{J1a} is independently —H, C₁₋₃ alkyl or C₁₋₄ haloalkyl;
or
R^{1a} and R^{1b}, together with the carbon atom to which they are attached form a C₃₋₆ cycloalkyl, or a 3- to 6-membered monocyclic heterocycle containing 1 ring heteroatom selected from O, N and S, wherein the 1 ring heteroatom is not bonded to the carbon to which R^{1a} and R^{1b} are attached;
   wherein each of said C₃₋₆ cycloalkyl and said 3- to 6-membered monocyclic heterocycle is unsubstituted or substituted with 1 or 2 substituents independently selected from halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, (C(R^{J1a}₂))₀₋₂—NH₂, —(C(R^{J1a}₂))₀₋₂—NHR^{J1}, and —(C(R^{J1a}₂))₀₋₂—NR^{J1}₂, or wherein two geminal substituents, together with the carbon atom to which they are attached, form a C₃₋₆ cycloalkyl or 3- to 6-membered monocyclic heterocycle containing 1-2 heteroatoms selected from O, N, and S,
      wherein each instance of R^{J1} is independently C₁₋₃ alkyl or C₁₋₄ haloalkyl,
      wherein each instance of R^{J1a} is independently —H, C₁₋₃ alkyl or C₁₋₄ haloalkyl;
R² is phenyl or 5- or 6-membered monocyclic heteroaryl having 1-3 ring heteroatoms independently selected from O, N and S,
   wherein each of said phenyl and said 5- or 6-membered monocyclic heteroaryl is unsubstituted or substituted with 1-3 substituents independently selected from halo, C₁₋₄ alkyl, C₁₋₄ haloalkyl, —(C(R^{J2a}₂))₀₋₂—OH, —(C(R^{J2a}₂))₀₋₂—OR^{J1}₂, —(C(R^{J2a}₂))₀₋₂—SR^{J2}, —(C(R^{J2a}₂))₀₋₂—NH₂, —(C(R^{J2a}₂))₀₋₂—NHR^{J2}, —(C(R^{J2a}₂))₀₋₂—NR^{J2}₂, —C(O)R^{J2}, and —CN,
      wherein each instance of R^{J2} is independently C₁₋₃ alkyl or C₁₋₄ haloalkyl,
      wherein each instance of R^{J2a} is independently —H, C₁₋₃ alkyl or C₁₋₄ haloalkyl,
      wherein optionally two adjacent substituents of said phenyl together form methylenedioxy, wherein the methylene unit of the methylenedioxy is unsubstituted or substituted with halo; and
R³ is phenyl, or 5- or 6-membered monocyclic heteroaryl having 1-4 ring heteroatoms independently selected from O, N and S,
   wherein each of said phenyl and said 5- or 6-membered monocyclic heteroaryl is unsubstituted or substituted with 1-3 substituents independently selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))_{0- 2}—NHR^{J3}, —(C(R^{J3a}₂))₀₋₂—NR^{J3}₂, —C(O)R^{J3}, and —CN,
      wherein each instance of R^{J3} is independently C₁₋₃ alkyl or C₁₋₄ haloalkyl, and
      wherein each instance of R^{J3a} is independently —H, C₁₋₃ alkyl or C₁₋₄ haloalkyl; each of R^{4a} and R^{4b} independently is —H, halo, C₁₋₄ alkyl and
Y is —NH— or —N(C₁₋₄ alkyl)-;
wherein 0 to 6 hydrogen atoms of said compound of Formula (I) are optionally replaced with deuterium;
provided that the compound of Formula (I) is not

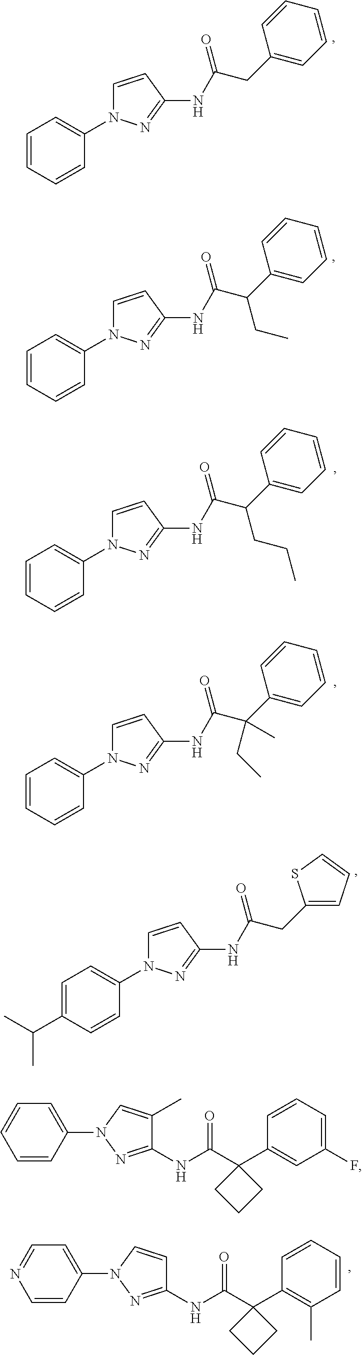

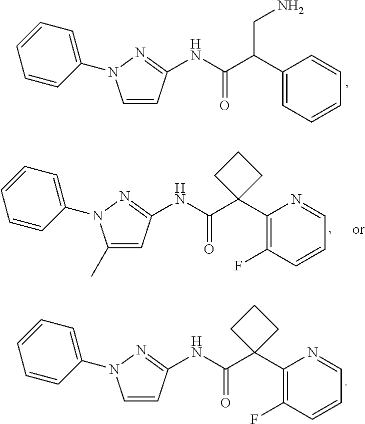

In some embodiments, each of R^{1a} and R^{1b} independently is —H, C₁₋₄ alkyl, C₁₋₄ haloalkyl, —(C(R^{J1a}₂))₁₋₂—OH, —(C(R^{J1a}₂))₁₋₂—OR^{J1}, —(C(R^{J1a}₂))₁₋₂—SR^{J1}, —(C(R^{J1a}₂))₁₋₂—NH₂, —(C(R^{J1a}₂))₁₋₂—NHR^{J1}, —(C(R^{J1a}₂))₁₋₂—NR^{J1}₂, C₃₋₆ cycloalkyl or a 3- to 6-membered monocyclic heterocycle containing 1 ring heteroatom selected from O, N, and S,

wherein the 3- to 6-membered monocyclic heterocycle does not contain a heteroatom bonded to the carbon to which R^{1a} and R^{1b} are attached,
wherein each instance of R^{J1} is independently C₁₋₃ alkyl or C₁₋₄ haloalkyl,
wherein each instance of R^{J1a} is independently —H, C₁₋₃ alkyl or C₁₋₄ haloalkyl;
or
R^{1a} and R^{1b}, together with the carbon atom to which they are attached form a C₃₋₆ cycloalkyl, or a 3- to 6-membered monocyclic heterocycle containing 1 ring heteroatom selected from O, N and S, wherein the 1 ring heteroatom is not bonded to the carbon to which R^{1a} and R^{1b} are attached;
wherein each of said C₃₋₆ cycloalkyl and said 3- to 6-membered monocyclic heterocycle is unsubstituted or substituted with 1 or 2 substituents independently selected from halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, —(C(R^{J1a}₂))₀₋₂—NH₂, —(C(R^{J1a}₂))₀₋₂—NHR^{J1}, and —(C(R^{J1a}₂))₀₋₂—NR^{J1}₂, or wherein two geminal substituents, together with the carbon atom to which they are attached, form a C₄₋₆ cycloalkyl or 4- to 6-membered monocyclic heterocycle containing 1-2 heteroatoms selected from O, N, and S

In some aspects, the present invention provides a pharmaceutical composition comprising a chemical entity described herein (i.e., free compound, a pharmaceutically acceptable salt thereof, or a mixture of free compound and pharmaceutically acceptable salt thereof) and a pharmaceutically acceptable carrier, adjuvant, or excipient.

In some aspects, the present invention provides a method for treating a disease, disorder or condition responsive to reduction of VLCFA levels in a patient comprising administering to the patient an effective amount of a chemical entity described herein. In some embodiments, the subject can be a mammal. In some embodiments, the subject can be a human. In some embodiments, the subject has ALD.

In some aspects, the present invention provides a method of treating, preventing, or ameliorating one or more symptoms of a subject with ALD, its phenotypes, or other disease, disorder or condition responsive to reduction of VLCFA levels in a subject. Examples of symptoms include, but are not limited to, decreased sensitivity to stimulus (e.g., in appendages and hands), seizures, coma, death, bladder misfunction, sphincter dysfunction, misfunction of gait, ability to walk, inability to see/hear, those associated with adrenal gland insufficiency (e.g., weakness/fatigue, nausea, abdominal pain, low blood pressure), or associated with peripheral neuropathy.

In some aspects, the present invention provides a method for reduction of VLCFA levels. In some embodiments, the reduction is reversible. In some embodiments, the reduction can be achieved in a cell (e.g., the cell used in an in vitro assay; cell in vitro; or cell ex vivo), the cell of a patient, by administering to the patient, or to the cell of the patient, or to a biological sample from the patient and comprising the cell, an effective amount of a chemical entity described herein. In some embodiments, the reduction can be achieved in a tissue, e.g., the tissue of a patient, by administering to the patient, or to the tissue of the patient, or to a biological sample from the patient and comprising the tissue, an effective amount of a chemical entity described herein. In certain embodiments, the tissue can be brain tissue, adrenal gland tissue, muscle tissue, nerve (e.g., peripheral nerve) tissue, adipose tissue, testes tissue, eye tissue, or liver tissue. In some embodiments, the reduction can be achieved in a biological fluid, e.g., the biological fluid of a patient, by administering to the patient, or to the biological fluid of the patient, or to a sample from the patient and comprising the biological fluid, an effective amount of a chemical entity described herein. In certain embodiments, the biological fluid can be cerebrospinal fluid (CSF), blood, or any fraction of blood, e.g., serum, or can be from the skin (e.g., skin oil).

In some aspects, the present invention provides methods of preparing the chemical entities of Formula (I), such as chemical entities of Formula (II), (III), (A), (B), (C), (1), (3), (II.A), (II.B), (II.C), (II.1), (III.A), (III.B), (III.C), (III.1), (A.1), (B.1), (C.1), (II.A.1), (II.B.1), (II.C.1), (III.A.1), (III.A.1a), (III.A.1b), (III.A.3), (III.B.1) and/or (III.C.1), including compounds described further herein.

### BRIEF DESCRIPTION OF THE DRAWINGS

FIG. 1 shows dose response in adrenoleukodystrophy (ALD) patient fibroblasts (AMN 1, CALD 1, AMN 2) and healthy human fibroblasts (Healthy 1, Healthy 2) (FIG. 1A), ALD patient B-lymphocytes (CALD 1, Heterozygous (Het) Female 1, Heterozygous (Het) Female 2) (FIG. 1B), and human microglia (FIG. 1C) with administration of Compound 87. In FIG. 1A, FIG. 1B, and FIG. 1C, the level of VLCFA, lysophosphatidylcholine (LPC), was measured in human fibroblast and lymphocyte cells (FIGS. 1A and 1B, respectively) from both ALD and healthy patients and in human microglia cells, each grown with ¹³C-acetate in the presence of increasing concentrations of Compound 87 for about 48 hr. The LPC level is depicted as C26:0 LPC/C16:0 LPC level, indicating that the C26:0 LPC measurement was normalized (i.e., divided by) the C16:0 LPC measurement, for example, as shown in FIG. 1A, FIG. 1B, and FIG. 1C, via mass spectroscopy. AMN: adrenomyeloneuropathy; AMN 1 are cells from one male patient and AMN 2 are cells from a different male patient; he CALD 1 cell line from which fibroblasts in FIG. 1A were derived is different from the CALD 1 cell line from which B-lymphocytes in FIG. 1B were derived; Het Female 1 are cells from one heterozygous female and Het Female 2 are cells from a different heterozygous female; healthy 1 and healthy 2 are control cell lines from two human fibroblast cell lines in which the humans do not have ABCD1 mutations.

FIG. 2 shows reduction of a VLCFA level, specifically C26:0 LPC level in vivo in blood following administration of Compound 87, from ABCD1 knockout (KO) mice, wild-type (WT) rats, and cynomolgous monkeys, each as further described below. ABCD1 KO mice received no treatment, vehicle (2% D-α-Tocopherol polyethylene glycol 1000 succinate (TPGS)), or 1, 8, or 16 mg/kg Compound 87 PO QD daily for 14 days (FIG. 2A). WT and ABCD1 KO mice received 0.5 to 64 mg/kg Compound 87 PO QD and LPC levels, depicted as C26:0 LPC/C16:0 LPC level, were examined after 28 days of dosing (FIG. 2B). WT rats received 2% TPGS vehicle or 30, 100, or 300 mg/kg Compound 87 PO QD for 7 days and LPC levels, depicted as C26:0 LPC/C16:0 LPC level, were examined (FIG. 2C). Male Cynomolgous monkeys received 30 mg/kg Compound 87 PO QD for 7 days and LPC levels, depicted as C26:0 LPC/C16:0 LPC level, were examined (FIG. 2D). Compound 87 was dosed PO QD at 1 and 10 mg/kg to adult female ABCD1 KO mice (n=6), with groups analyzed at 3 months and, as shown, and LPC levels, depicted as C26:0 LPC/C16:0 LPC level, in the blood were maintained at near WT levels through 3 months dosing (FIG. 2E; P values versus ABCD1 KO vehicle controls (*** P≤0.001, **** P≤0.0001); error bars indicate standard deviation). Discontinuation of Compound 87 returns blood LPC levels, depicted as C26:0 LPC/C16:0 LPC level, to about baseline level in adult female ABCD1 KO mice (n=5) (FIG. 2F; error bars indicate standard deviation. For FIG. 2A to FIG. 2F, the vehicle used was 2% D-α-Tocopherol polyethylene glycol 1000 succinate (TPGS) and Compound 87 doses were prepared in 2% TPGS. As used herein, mpk means mg/kg.

FIG. 3 shows reduction of VLCFA level, specifically C24:0 LPC level and C26:0 LPC level, in the brain following administration of Compound 87 in adult female ABCD1 KO mice. ABCD1 KO mice received vehicle (n=6), 1 mg/kg Compound 87 (n=6), or 10 mg/kg Compound 87 (n=6) PO QD for 3 months. WT mice also received vehicle for 3 months (n=6). Ten mg/kg Compound 87 in ABCD1 KO mice induced significant reduction in brain C24:0 LPC (FIG. 3E) and in brain C26:0 LPC level (about 40% reduction for C26:0 LPC level) (FIG. 3F), with 1 mg/kg Compound 87 showing about a 30% reduction in brain C26:0 LPC level, each after 3 months of dosing. Levels of other LPC are shown for comparison (FIG. 3A: C16:0 LPC; FIG. 3B: C18:0 LPC; FIG. 3C: C20:0 LPC; FIG. 3D: C22:0 LPC). Data shown for C18:0, C20:0, C22:0, C24:0, and C26:0 LPCs were normalized by the C16:0 LPC signal counts. P values versus ABCD1 KO vehicle controls are indicated as follows: *P≤0.05, ** P≤0.01, *** P≤0.001, **** P≤0.0001; error bars indicate standard deviation.

FIG. 4 shows reduction of VLCFA level, specifically C24:0 SC-VLCFA level and C26:0 SC-VLCFA level, in the brain following administration of Compound 87, in wild-type mice (n=6) and adult female ABCD1 KO mice (n=6) for 3 months. Mice received vehicle (2% TPGS), 1 mg/kg Compound 87 or 10 mg/kg Compound 87 PO QD for 3 months. Ten mg/kg Compound 87 induced a significant reduction in brain C24:0 SC-VLCFA level and in brain C26:0 SC-VLCFA level (about a 65% reduction in brain C26:0 VLCFA level), each after 3 months of dosing (** P≤0.01, **** P≤0.0001, respectively) (FIG. 4E and FIG. 4F, respectively). Levels of other VLCFA are shown for comparison (FIG. 4A: C16:0 VLCFA; FIG. 4B: C18:0 VLCFA; FIG. 4C: C20:0 VLCFA; FIG. 4D: C22:0 VLCFA).

FIG. 5 shows the response latency (in seconds) of male ABCD1 KO mice that received prophylactic or therapeutic dosing of Compound 87 in response to an infrared source on each hind paw. FIG. 5A shows the response latency from the prophylactic dosing of Compound 87 PO QD at 5 mg/kg (data shown with squares), Compound 87 PO QD at 20 mg/kg (data shown with triangles), and 2% TPGS vehicle (data shown with circles) (n=8-10 mice per group). FIG. 5B shows the response latency from the therapeutic dosing of Compound 87 PO QD at 32 mg/kg (data shown with squares), Compound 87 PO QD at 64 mg/kg (data shown with triangles), and 2% TPGS vehicle (data shown with circles) (n=8-10 mice per group). In FIG. 5A and FIG. 5B, the dashed line indicates historical WT mouse responses, error bars indicate standard error of the mean, and * corresponds to Tukey's post-hoc test between groups and indicates a significant difference from vehicle treated mice during that month.

### DETAILED DESCRIPTION

### Chemical Entities

As used herein, the term “chemical entity” refers to a compound having a structure identified by a specific or generic structural formula, and/or a pharmaceutically acceptable salt thereof. When a salt form is specifically intended, the term “pharmaceutically acceptable salt” is used. When a non-salt form is specifically intended, the term “free compound”, or a variant such as “free acid” or “free base”, is used. The term “compound” is used herein variously to refer to a chemical entity or specifically to a free compound or a pharmaceutically acceptable salt, as informed by context. Thus, statements herein regarding “compounds” apply equally to chemical entities and, as applicable, vice-versa. Accordingly, no significance is intended by the use of “chemical entity” in some contexts and “compound” in others with respect to the description of the compound. For example, a reference to “compounds of Tables A-E” or “compounds of Table 1” is intended to include both free compounds and salt forms, unless otherwise specified or clear from context.

As used herein, the term “a free compound of formula (n),” where “(n)” refers to any Formula or embodiments thereof described herein (e.g., Formula (I), including one or more of Formula (II), (III), (A), (B), (C), (1), (3), (II.A), (II.B), (II.C), (II.1), (III.A), (III.B), (III.C), (III.1), (A.1), (B.1), (C.1), (II.A.1), (II.B.1), (II.C.1), (III.A.1), (III.A.1a), (III.A.1b), (III.A.3), (III.B.1) and/or (III.C.1), and embodiments thereof) refers to the non-salt form, i.e., free base, free acid, or neutral form which is not a salt unless otherwise specified. For example, a free base or free acid compound may comprise an ionizable group (e.g., a basic nitrogen or an acidic group such as a carboxylic acid or phenol) that is in neutral form and not ionized (e.g., to form a pharmaceutically acceptable salt of a free base or free acid compound).

As used herein, the term “a pharmaceutically acceptable salt of a free compound of Formula (n)” means a compound of Formula (n) in a pharmaceutically acceptable salt form unless otherwise specified. For example, when a free compound comprises an ionizable group (e.g., a basic nitrogen or an acidic group such as a carboxylic acid or phenol) that is ionized, a pharmaceutically acceptable salt of the free compound can be formed which has a suitable counterion.

The chemical entities provided herein can be useful for reduction of VLCFA levels or for treating disorders related to impaired peroxisomal function (e.g., impaired transport of VLCFA into the peroxisomes or impaired VLCFA degradation/metabolism within the peroxisomes) or accumulation of very long-chain fatty acids (VLCFA). In some embodiments, the chemical entities are useful for treating disorders associated with deficiency or mutations of at least one of ABCD1 protein (also known as ALD protein), Acyl-CoA Binding Domain Containing 5 (ACBD5), Acyl-CoA oxidase (e.g., ACOX1), or D-Bifunctional protein (DBP). In some embodiments, the chemical entities are useful for treating ALD and its phenotypes (e.g., CALD and AMN). In some embodiments, the chemical entities are useful for treating CALD. In some embodiments, the chemical entities are useful for treating AMN. In some embodiments, the chemical entities are useful for treating Zellweger spectrum disorders (ZSD; peroxisomal biogenesis disorders).

In some aspects, provided is a chemical entity, which is a free compound represented by Formula (I), e.g., represented by Formula (II), (III), (A), (B), (C), (1), (3), (II.A), (II.B), (II.C), (II.1), (III.A), (III.B), (III.C), (III.1), (A.1), (B.1), (C.1), (II.A.1), (II.B.1), (II.C.1), (III.A.1), (III.A.1a), (III.A.1b), (III.A.3), (III.B.1) and/or (III.C.1), or a pharmaceutically acceptable salt thereof, wherein the variables are each and independently as described herein. In some embodiments, a chemical entity is a free compound of any of the foregoing Formulas or a pharmaceutically acceptable salt thereof. In some embodiments, a chemical entity is a free compound of any of the foregoing Formulas. In some embodiments, a chemical entity is a pharmaceutically acceptable salt of a free compound of any of the foregoing Formulas.

In some embodiments, a chemical entity is a free compound of formula (I), a pharmaceutically acceptable salt of a free compound of formula (I), a pharmaceutically acceptable prodrug of a free compound of formula (I), or a pharmaceutically acceptable metabolite of a free compound of formula (I). In some embodiments, a chemical entity is a non-covalent complex between a free compound of formula (I) or a pharmaceutically acceptable salt thereof and another compound. In some embodiments, a non-covalent complex is a solvate (e.g., a hydrate) of a free compound of formula (I) or a pharmaceutically acceptable salt thereof. In some embodiments, a non-covalent complex is a chelate of a free compound of formula (I) or a pharmaceutically acceptable salt thereof. In some embodiments, a non-covalent complex comprises a conformer and a free compound of formula (I) or a pharmaceutically acceptable salt thereof.

Unless otherwise specified or clear from context, a chemical entity can be in any solid form, i.e., amorphous or crystalline (e.g., polymorphs), or combinations of solid forms (e.g., combination of at least two crystalline compounds or combination of at least one crystalline compound and at least one amorphous compound). In some embodiments, a chemical entity is a crystalline compound. In some embodiments, a chemical entity is an amorphous compound. In some embodiments, a chemical entity is a mixture of crystalline compounds. In some embodiments, a chemical entity is a mixture of at least one crystalline compound and at least one amorphous compound.

In some embodiments, a provided chemical entity is a free compound of Formula (II) or a pharmaceutically acceptable salt thereof, wherein Formula (II) has the structure,

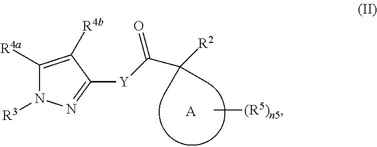

wherein:
A is a C₃₋₆ cycloalkyl or a 4- to 6-membered monocyclic heterocycle containing 1 ring heteroatom selected from O, N and S; wherein the 1 ring heteroatom is not bonded to the carbon to which A is attached;
each instance of R⁵ independently is selected from halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, —(C(R^{J1a}₂))₀₋₂—NH₂, —(C(R^{J1a}₂))₀₋₂—NHR^{J1}, and —(C(R^{J1a}₂))₀₋₂—NR^{J1}₂,
   wherein each instance of R^{J1} is independently C₁₋₃ alkyl or C₁₋₄ haloalkyl, and
   wherein each instance of R^{J1a} is independently —H, C₁₋₃ alkyl or C₁₋₄ haloalkyl;
or two geminal R⁵, together with the carbon atom to which they are attached, form a C₃₋₆ cycloalkyl or 3- to 6-membered monocyclic heterocycle containing 1-2 heteroatoms independently selected from O, N, and S;
n5 is 0, 1 or 2; and
each of R², R³, R^{4a}, R^{4b} and Y is as defined above for Formula (I), both singly and in combination.

In some embodiments, A is cyclopropyl, cyclobutyl or oxetanyl.

In some embodiments, a provided chemical entity is a free compound of Formula (III) or a pharmaceutically acceptable salt thereof, wherein Formula (III) has the structure,

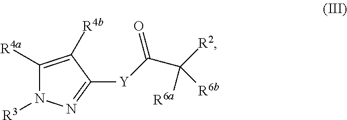

wherein:
each of R^{6a} and R^{6b} independently is —H, C₁₋₄ alkyl, C₁₋₄ haloalkyl, —(C(R^{J1a}₂))₁₋₂—OH, —(C(R^{J1a}₂))₁₋₂—OR^{J1}, —(C(R^{J1a}₂))₁₋₂—SR^{J1}, —(C(R^{J1a}₂))₁₋₂—NH₂, —(C(R^{J1a}₂))₁₋₂—NHR^{J1}, —(C(R^{J1a}₂))₁₋₂—NR^{J1}₂, C₃₋₆ cycloalkyl, or a 3- to 6-membered heterocycle containing 1 ring heteroatom selected from O, N, and S,
   wherein the 3- to 6-membered monocyclic heterocycle does not contain a heteroatom bonded to the carbon to which R^{1a} and R^{1b} are attached,
   wherein each instance of R^{J1} is independently C₁₋₃ alkyl or C₁₋₄ haloalkyl, and
   wherein each instance of R^{J1a} is independently —H, C₁₋₃ alkyl or C₁₋₄ haloalkyl; and
each of R², R³, R^{4a}, R^{4b} and Y is as defined above for Formula (I), both singly and in combination.

In some embodiments, a provided chemical entity is a free compound of Formula (A) or a pharmaceutically acceptable salt thereof, wherein Formula (A) has the structure,

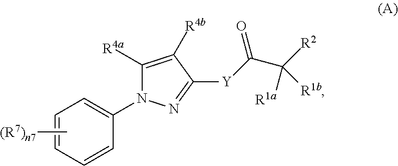

wherein:
each instance of R⁷ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, (C(R^{J3a}₂))₀₋₂—NH₂, —(C(R^{J3a}₂))₀₋₂—NHR^{J3}, (C(R^{J3a}₂))_{0- 2}—NR^{J3}₂, —C(O)R^{J3}, and —CN,
   wherein each instance of R^{J3} is independently C₁₋₃ alkyl or C₁₋₄ haloalkyl, and
   wherein each instance of R^{J3a} is independently —H, C₁₋₃ alkyl, C₁₋₄ haloalkyl;
n7 is 0, 1, 2 or 3; and
each of R^{1a}, R^{1b}, R², R^{4a}, R^{4b} and Y is as defined above for Formula (I), both singly and in combination.

In some embodiments, a provided chemical entity is a free compound of Formula (B) or a pharmaceutically acceptable salt thereof, wherein Formula (B) has the structure,

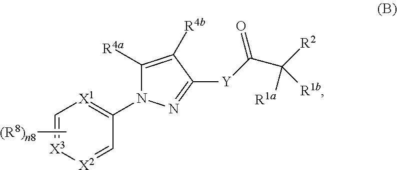

wherein:
one of X¹, X² and X³ is N, and the other two are carbon atoms;
each instance of R⁸ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, (C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
   wherein each instance of R^{J3} is independently C₁₋₃ alkyl or C₁₋₄ haloalkyl, and
   wherein each instance of R^{J3a} is independently —H, C₁₋₃ alkyl, or C₁₋₄ haloalkyl;
n8 is 0, 1, 2 or 3; and
each of R^{1a}, R^{1b}, R², R^{4a}, R^{4b} and Y is as defined above for Formula (I), both singly and in combination.

In some embodiments, a provided compound is a compound of Formula (B) in which X¹ is N, and X² and X³ are carbon atoms. In some embodiments, a provided compound is a compound of Formula (B) in which X² is N, and X¹ and X³ are carbon atoms. In some embodiments, a provided compound is a compound of Formula (B) in which X³ is N, and X¹ and X² are carbon atoms.

In some embodiments, a provided chemical entity is a free compound of Formula (C) or a pharmaceutically acceptable salt thereof, wherein Formula (C) has the structure,

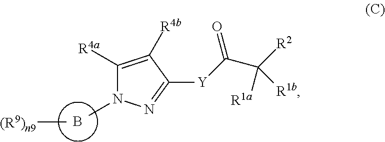

wherein:
B is 5-membered monocyclic heteroaryl having 1-4 ring heteroatoms independently selected from O, N and S, or 6-membered monocyclic heteroaryl having 2 or 3 ring nitrogen atoms; each instance of R⁹ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
   wherein each instance of R^{J3} is independently C₁₋₃ alkyl or C₁₋₄ haloalkyl, and
   wherein each instance of R^{J3a} is independently —H, C₁₋₃ alkyl or C₁₋₄ haloalkyl;
n9 is 0, 1, 2 or 3; and
each of R^{1a}, R^{1b}, R², R^{4a}, R^{4b} and Y is as defined above for Formula (I), both singly and in combination.

In some embodiments, a provided chemical entity is a free compound of Formula (1) or a pharmaceutically acceptable salt thereof, wherein Formula (1) has the structure,

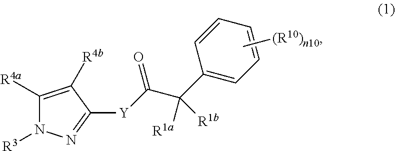

wherein:
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J2a}₂))₀₋₂—SR^{J2}, —(C(R^{J2a}₂))₀₋₂—NH₂, —(C(R^{J2a}₂))₀₋₂—NHR^{J2}, —(C(R^{J2a}₂))₀₋₂—NR^{J2}₂, —C(O)R^{J2}, and —CN, or two adjacent R¹⁰ form methylenedioxy, wherein the methylene unit of the methylenedioxy is unsubstituted or substituted with halo;
   wherein each instance of R^{J2} is independently C₁₋₃ alkyl or C₁₋₄ haloalkyl, and
   wherein each instance of R^{J2a} is independently —H, C₁₋₃ alkyl or C₁₋₄ haloalkyl;
n10 is 0, 1, 2 or 3; and
each of R^{1a}, R^{1b}, R³, R^{4a}, R^{4b} and Y is as defined above for Formula (I), both singly and in combination.

In some embodiments, a provided chemical entity is a free compound of Formula (3) or a pharmaceutically acceptable salt thereof, wherein Formula (3) has the structure,

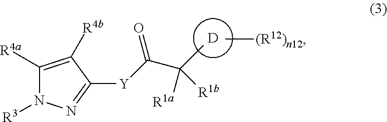

wherein:
D is 5- or 6-membered monocyclic heteroaryl having 1-3 ring heteroatoms independently selected from O, N and S;
each instance of R¹² independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J2a}₂))₀₋₂—SR^{J2}, —(C(R^{J2a}₂))₀₋₂—NH₂, —(C(R^{J2a}₂))₀₋₂—NHR^{J1}₂, —(C(R^{J2a}₂))₀₋₂—NR^{J2}₂, —C(O)R^{J1}₂, and —CN, or two adjacent R¹⁰ form methylenedioxy, wherein the methylene unit of the methylenedioxy is unsubstituted or substituted with halo;
   wherein each instance of R^{J2} is independently C₁₋₃ alkyl or C₁₋₄ haloalkyl, and
   wherein each instance of R^{J2a} is independently —H, C₁₋₃ alkyl or C₁₋₄ haloalkyl;
n12 is 0, 1, 2 or 3; and
each of R^{1a}, R^{1b}, R³, R^{4a}, R^{4b} and Y is as defined above for Formula (I), both singly and in combination.

In some embodiments, a provided chemical entity is a free compound of Formula (II.A) or a pharmaceutically acceptable salt thereof, wherein Formula (II.A) has the structure,

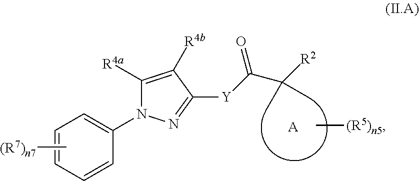

wherein A, R⁵, n5, R², R^{4a}, R^{4b}, Y, R⁷ and n7 are as defined above for Formulas (II) and (A), both singly and in combination.

In some embodiments, a provided chemical entity is a free compound Formula (II.B) or a pharmaceutically acceptable salt thereof, wherein Formula (II.B) has the structure,

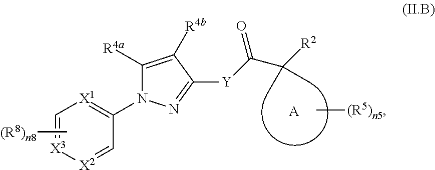

wherein A, R⁵, n5, R², R^{4a}, R^{4b}, Y, X¹, X², X³, R⁸ and n8 are as defined above for Formulas (II) and (B), both singly and in combination.

In some embodiments, a provided chemical entity is a free compound of Formula (II.C) or a pharmaceutically acceptable salt thereof, wherein Formula (II.C) has the structure,

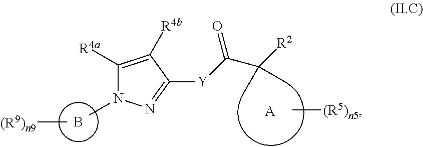

wherein A, R⁵, n5, R², R^{4a}, R^{4b}, Y, B, R⁹ and n9 are as defined above for Formulas (II) and (C), both singly and in combination.

In some embodiments, a provided chemical entity is a free compound of Formula (II.1) or a pharmaceutically acceptable salt thereof, wherein Formula (II.1) has the structure,

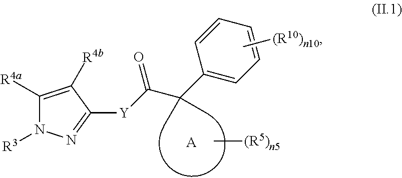

wherein A, R⁵, n5, R³, R^{4a}, R^{4b}, Y, R¹⁰ and n10 are as defined above for Formulas (II) and (1), both singly and in combination.

In some embodiments, a provided chemical entity is a free compound of Formula (III.A) or a pharmaceutically acceptable salt thereof, wherein Formula (III.A) has the structure,

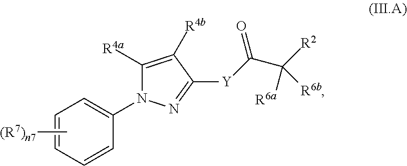

wherein R^{6a}, R^{6b}, R², R^{4a}, R^{4b}, Y, R⁷ and n7 are as defined above for Formulas (III) and (A), both singly and in combination.

In some embodiments, a provided chemical entity is a free compound of Formula (III.B) or a pharmaceutically acceptable salt thereof, wherein Formula (III.B) has the structure,

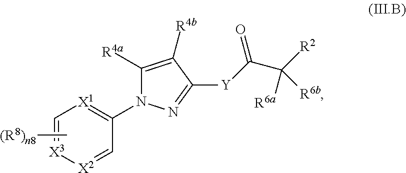

wherein R^{6a}, R^{6b}, R², R^{4a}, R^{4b}, Y, X¹, X², X³, R⁵ and n8 are as defined above for Formulas (III) and (B), both singly and in combination.

In some embodiments, a provided chemical entity is a free compound of Formula (III.C) or a pharmaceutically acceptable salt thereof, wherein Formula (III.C) has the structure,

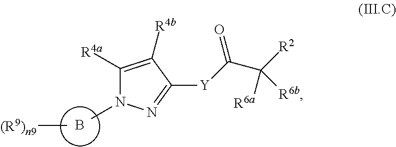

wherein R⁶, R^{6b}, R², R^{4a}, R^{4b}, Y, B, R⁹ and n9 are as defined above for Formulas (III) and (C), both singly and in combination.

In some embodiments, a provided chemical entity is a free compound of Formula (III.1) or a pharmaceutically acceptable salt thereof, wherein Formula (III.1) has the structure,

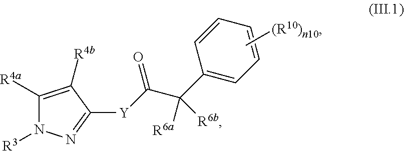

wherein R^{6a}, R^{6b}, R³, R^{4a}, R^{4b}, Y, R¹⁰ and n10 are as defined above for Formulas (III) and (1), both singly and in combination.

In some embodiments, a provided chemical entity is a free compound of Formula (A.1) or a pharmaceutically acceptable salt thereof, wherein Formula (A.1) has the structure,

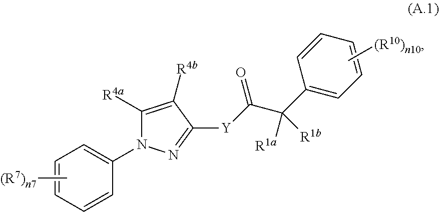

wherein R⁷, n7, R^{1a}, R^{1b}, R^{4a}, R^{4b}, Y, R¹⁰ and n10 are as defined above for Formulas (A) and (1), both singly and in combination.

In some embodiments, a provided chemical entity is a free compound of Formula (B.1) or a pharmaceutically acceptable salt thereof, wherein Formula (B.1) has the structure,

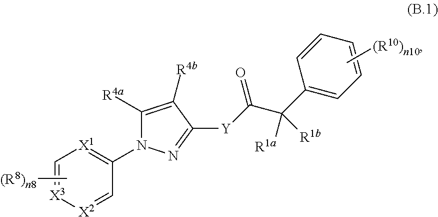

wherein R⁸, n8, X¹, X², X³, R^{1a}, R^{1b}, R^{4a}, R^{4b}, Y, R¹⁰ and n10 are as defined above for Formulas (B) and (1), both singly and in combination.

In some embodiments, a provided chemical entity is a free compound of Formula (C.1) or a pharmaceutically acceptable salt thereof, wherein Formula (C.1) has the structure,

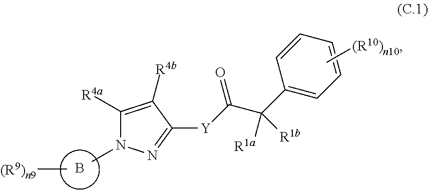

wherein B, R⁹, n9, R^{1a}, R^{1b}, R^{4a}, R^{4b}, Y, R¹⁰ and n10 are as defined above for Formulas (C) and (1), both singly and in combination.

In some embodiments, a provided chemical entity is a free compound of Formula (II.A.1) or a pharmaceutically acceptable salt thereof, wherein Formula (II.A.1) has the structure,

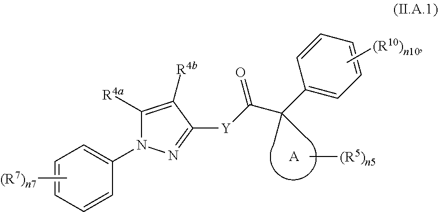

wherein:
A is a C₃₋₆ cycloalkyl or a 3- to 6-membered monocyclic heterocycle containing 1 ring heteroatom selected from O, N and S, wherein the 1 ring heteroatom is not bonded to the carbon to which A is attached;
each instance of R⁵ independently is selected from halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, —(C(R^{J1a}₂))₀₋₂—NH₂, —(C(R^{J1a}₂))₀₋₂—NHR^{J1}, and —(C(R^{J1a}₂))₀₋₂—NR^{J1}₂,
   wherein each instance of R^{J1} is independently C₁₋₃ alkyl or C₁₋₄ haloalkyl, and
   wherein each instance of R^{J1a} is independently —H, C₁₋₃ alkyl or C₁₋₄ haloalkyl;
or two geminal R⁵, together with the carbon atom to which they are attached, form a C₄₋₆ cycloalkyl or 4- to 6-membered monocyclic heterocycle containing 1-2 heteroatoms independently selected from O, N, and S;
n5 is 0, 1 or 2;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J2a}₂))₀₋₂—SR^{J2}, —(C(R^{J2a}₂))₀₋₂—NH₂, —(C(R^{J2a}₂))₀₋₂—NHR^{J2}, —(C(R^{J2a}₂))₀₋₂—NR^{J2}₂, —C(O)R^{J2}, and —CN, or two adjacent R¹⁰ form methylenedioxy, wherein the methylene unit of the methylenedioxy is unsubstituted or substituted with halo;
   wherein each instance of R^{J2} is independently C₁₋₃ alkyl or C₁₋₄ haloalkyl, and
   wherein each instance of R^{J2a} is independently —H, C₁₋₃ alkyl or C₁₋₄ haloalkyl;
n10 is 0, 1, 2 or 3;
each instance of R⁷ independently is selected from halo, C₁₋₄ alkyl, C₁₋₄ haloalkyl, —(C(R^{J3a}₂))₀₋₂—OH, —(C(R^{J3a}₂))₀₋₂—OR³, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
   wherein each instance of R^{J3} is independently C₁₋₃ alkyl or C₁₋₄ haloalkyl, and
   wherein each instance of R^{J3a} is independently —H, C₁₋₃ alkyl or C₁₋₄ haloalkyl;
n7 is 0, 1, 2 or 3;
each of R^{4a} and R^{4b} independently is —H, halo or C₁₋₄ alkyl; and Y is —NH— or —N(C₁₋₄ alkyl)-.

In some embodiments, A is cyclopropane, cyclobutane, cyclopentane, cyclohexane, azetidine, oxetane, pyrrolidine, tetrahydrofuran, tetrahydrothiophene, piperidine, tetrahydropyran or tetrahydrothiopyran, wherein the heteroatom of each of the foregoing applicable rings is not bonded to the carbon to which A is attached. In some embodiments, A is cyclopropane, cyclobutane, cyclopentane, cyclohexane, pyrrolidine, oxetane or tetrahydropyran, wherein the heteroatom of each of the foregoing applicable rings is not bonded to the carbon to which A is attached. In some embodiments, A is pyrrolidine, oxetane or tetrahydropyran, wherein the heteroatom of each of the foregoing rings is not bonded to the carbon to which A is attached. In some embodiments, A is cyclopropane or cyclobutane. In some embodiments, A is cyclopropane. In some embodiments, A is one of the foregoing embodiments and is unsubstituted. In some embodiments, A is one of the foregoing embodiments and is substituted with 1-2 instances of R⁵ as defined herein for Formula (II).

In some embodiments, each instance of R⁵ independently is halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, or two geminal R⁵, together with the carbon atom to which they are attached, form a C₄₋₆ cycloalkyl or 4- to 6-membered monocyclic heterocycle containing 1-2 heteroatoms independently selected from O, N, and S. In some embodiments, each instance of R⁵ independently is -D, halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, or two geminal R⁵, together with the carbon atom to which they are attached, form a C₄₋₆ cycloalkyl. In some embodiments, each instance of R⁵ independently is -D, halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, —(C(R^{J1a}₂))₀₋₂—NH₂, —(C(R^{J1a}₂))₀₋₂—NHR^{J1}, or —(C(R^{J1a}₂))₀₋₂—NR^{J1}₂. In some embodiments, each instance of R⁵ independently is halo, C₁₋₄ alkyl, C₁₋₄ haloalkyl, —OH, or —NH₂. In some embodiments, two geminal R⁵, together with the carbon atom to which they are attached, form a C₄₋₆ cycloalkyl. In some embodiments, two geminal R⁵, together with the carbon atom to which they are attached, form a 4- to 6-membered monocyclic heterocycle containing 1-2 heteroatoms independently selected from O, N, and S. In some embodiments, two geminal R⁵, together with the carbon atom to which they are attached, form cyclobutane or cyclopentane. In some embodiments, each instance of R⁵ independently is C₁₋₄ alkyl. In some embodiments, each instance of R⁵ is Me. In some embodiments, each instance of R⁵ independently is Me or Et. In some embodiments, each instance of R⁵ independently is halo. In some embodiments, each instance of R⁵ independently is —F or —Cl.

In some embodiments n5 is 0, 1 or 2. In some embodiments, n5 is 0. In some embodiments, n5 is 2 and (R⁵)ₙ₅ is geminal di-(C₁₋₄ alkyl) or geminal di-halo. In some embodiments, n5 is 2 and (R⁵)ₙ₅ is geminal dimethyl. In some embodiments, n5 is 2 and (R⁵)ₙ₅ is geminal methyl and ethyl. In some embodiments, n5 is 2 and (R⁵)ₙ₅ is geminal difluoro or geminal dichloro. In some embodiments, n5 is 2 and two geminal R⁵, together with the carbon atom to which they are attached, form cyclobutane or cyclopentane.

In some embodiments, A is cyclopropane, cyclobutane or cyclopentane; n5 is 2; and (R⁵)ₙ₅ is geminal dimethyl, geminal difluoro or geminal dichloro. In some embodiments, A is cyclopropane, cyclobutane or cyclopentane, and n5 is 0. In some embodiments, A is cyclopropane or cyclobutane, and n5 is 0. The foregoing embodiments for A, R⁵, and n5 are also applicable to Formula (II), (II.A), (II.B), (II.C), (II.1), (II.B.1), and (II.C.1).

In some embodiments, each instance of R¹⁰ independently is —F, —Cl, —I, Me, Et, Pr, Bu, iPr, iBu, —OH, —OMe, —OEt, —OPr, —OiPr, NH₂, —NHMe, —NHEt, —NHiPr, —OCF₃, —CF₃, —CHF₂ or —CN, —SO₂NH₂, or two adjacent R¹⁰ form methylenedioxy wherein the methylene unit of the methylenedioxy is unsubstituted or substituted with halo. In some embodiments, each instance of R¹⁰ independently is —F, —Cl, Me, —OMe, —OEt, —CN or —CF₃. In some embodiments, each instance of R¹⁰ independently is —F, —Cl or —CF₃. In some embodiments, each instance of R¹⁰ is —F.

In some embodiments, n10 is 0 or 1, and R¹⁰ is —F, —Cl, Me, —OMe, —OEt, —CN or —CF₃. In some embodiments, n10 is 0. In some embodiments, n10 is 1 and R¹⁰ is —F.

In some embodiments, each of R^{4a} and R^{4b} independently is —H, Me, Et, Pr, Bu, ⁱPr, or ⁱBu. In some embodiments, R^{4a} is H and R^{4b} is Me. In some embodiments, R^{4a} is —H. In some embodiments R^{4b} is —H. In some embodiments, each of R^{4a} and R^{4b} is —H.

In some embodiments, each instance of R⁷ independently is —F, —Cl, Me, Et, Pr, Bu, iPr, iBu, —OH, —OMe, —OEt, —OPr, —OiPr, —NH₂, —NHMe, —NHEt, NHⁱPr, —CF₃, —CHF₂, —CN, or —SO₂NH₂. In some embodiments, each instance of R⁷ independently is —F, —Cl, or —CF₃. In some embodiments, each instance of R⁷ is —F.

In some embodiments, n7 is 0 or 1, and R⁷ is —F, —Cl or —CF₃. In some embodiments, n7 is 0.

In some embodiments, Y is —NH— or —N(Me)-. In some embodiments, Y is —NH—. In some embodiments, Y is —N(Me)-.

In some embodiments (II.A.1′), 1, 2, 3, 4, 5, or 6 instances of —H are replaced with -D (i.e., deuterium, —²H). In some embodiments, 1, 2, 3 or 4 instances of —H are replaced with -D. In some embodiments, at least one instance of -D is present in R^{4a} or R^{4b}. In some embodiments, at least one of R^{4a} and R^{4b} is -D. In some embodiments, R^{4a} is -D. In some embodiments, R^{4b} is -D. In some embodiments, at least one instance of -D is present in R⁵. In some embodiments, at least one instance of -D is present on A. In some embodiments, at least one instance of -D is present in R⁷. In some embodiments, at least one instance of -D is present on the ring to which R⁷ is attached. In some embodiments, at least one instance of -D is present in R¹⁰. In some embodiments, at least one instance of -D is present on the ring to which R¹⁰ is attached.

In some embodiments, each instance of R⁵ independently is halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, —(C(R^{J1a}₂))₀₋₂—NH₂, —(C(R^{J1a}₂))₀₋₂—NHR^{J1} or —(C(R^{J1a}₂))₀₋₂—NR^{J1}₂, or two geminal R⁵, together with the carbon atom to which they are attached, form a C₄₋₆ cycloalkyl, or at least one instance of -D is present on A. In some embodiments, each instance of R⁵ independently is halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, —(C(R^{J1a}₂))₀₋₂—NH₂, —(C(R^{J1a}₂))₀₋₂—NHR^{J1} or —(C(R^{J1a}₂))₀₋₂—NR^{J1}₂, or at least one instance of -D is present on A. In some embodiments, each instance of R⁵ independently is halo, C₁₋₄ alkyl, C₁₋₄ haloalkyl, —OH or —NH₂, or at least one instance of -D is present on A.

In some embodiments, each instance of R⁷ independently is —F, —Cl, Me, Et, Pr, Bu, iPr, iBu, —OH, —OMe, —OEt, —OPr, —OiPr, —NH₂, —NHMe, —NHEt, NHⁱPr, —CF₃, —CHF₂, —CN, or —SO₂NH₂, or at least one instance of -D is present on the ring to which R⁷ is attached.

In some embodiments, a provided chemical entity is a free compound of Formula (II.B.1) or a pharmaceutically acceptable salt thereof, wherein Formula (II.B.1) has the structure,

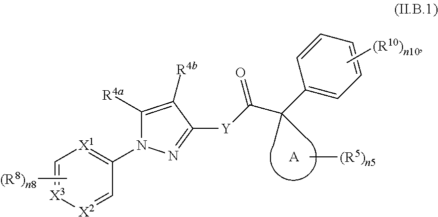

wherein:
A is a C₃₋₆ cycloalkyl or a 3- to 6-membered monocyclic heterocycle containing 1 ring heteroatom selected from O, N and S, wherein the 1 ring heteroatom is not bonded to the carbon to which A is attached;
each instance of R⁵ independently is selected from halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, —(C(R^{J1a}₂))₀₋₂—NH₂, —(C(R^{J1a}₂))₀₋₂—NHR^{J1}, and —(C(R^{J1a}₂))₀₋₂—NR^{J1}₂,
or two geminal R⁵, together with the carbon atom to which they are attached, form a C₄₋₆ cycloalkyl or 4- to 6-membered monocyclic heterocycle containing 1-2 heteroatoms independently selected from O, N, and S,
   wherein each instance of R^{J1} is independently C₁₋₃ alkyl or C₁₋₄ haloalkyl, and
   wherein each instance of R^{J1a} is independently —H, C₁₋₃ alkyl or C₁₋₄ haloalkyl;
n5 is 0, 1 or 2;
one of X¹, X² and X³ is N, and the other two are carbon atoms;
each instance of R⁸ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, (C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
   wherein each instance of R^{J3} is independently C₁₋₃ alkyl or C₁₋₄ haloalkyl, and
   wherein each instance of R^{J3a} is independently —H, C₁₋₃ alkyl or C₁₋₄ haloalkyl;
n8 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J2a}₂))₀₋₂—SR^{J2}, —(C(R^{J2a}₂))₀₋₂—NH₂, —(C(R^{J2a}₂))₀₋₂—NHR^{J1}₂, —(C(R^{J2a}₂))₀₋₂—NR^{J2}₂, —C(O)R^{J1}₂, and —CN, or two adjacent R¹⁰ form methylenedioxy, wherein the methylene unit of the methylenedioxy is unsubstituted or substituted with halo;
   wherein each instance of R^{J2} is independently C₁₋₃ alkyl or C₁₋₄ haloalkyl, and
   wherein each instance of R^{J2a} is independently —H, C₁₋₃ alkyl or C₁₋₄ haloalkyl;
n10 is 0, 1, 2 or 3;
each of R^{4a} and R^{4b} independently is —H, halo or C₁₋₄ alkyl; and
Y is —NH— or —N(C₁₋₄ alkyl)-.

In some embodiments, A is cyclopropane, cyclobutane, cyclopentane, cyclohexane, azetidine, oxetane, pyrrolidine, tetrahydrofuran, tetrahydrothiophene, piperidine, tetrahydropyran or tetrahydrothiopyran, wherein the heteroatom of each of the foregoing applicable rings is not bonded to the carbon to which A is attached. In some embodiments, A is cyclopropane, cyclobutane, cyclopentane, cyclohexane, oxetane or tetrahydropyran, wherein the heteroatom of each of the foregoing applicable rings is not bonded to the carbon to which A is attached. In some embodiments, A is oxetane, tetrahydrofuran, or tetrahydropyran, wherein the heteroatom of each of the foregoing rings is not bonded to the carbon to which A is attached. In some embodiments, A is cyclopropane or cyclobutane. In some embodiments, A is cyclopropane. In some embodiments, A is one of the foregoing embodiments and is unsubstituted. In some embodiments, A is one of the foregoing embodiments and is substituted with 1-2 instances of R⁵ as defined herein for Formula (II).

In some embodiments n5 is 0, 1 or 2. In some embodiments, n5 is 0. In some embodiments, n5 is 1. In some embodiments, n5 is 2. In some embodiments, n5 is 2 and (R⁵)ₙ₅ is geminal di-(C₁₋₄ alkyl) or geminal di-halo. In some embodiments, n5 is 2 and (R⁵)ₙ₅ is geminal dimethyl. In some embodiments, n5 is 2 and (R⁵)ₙ₅ is geminal difluoro or geminal dichloro. In some embodiments, n5 is 2 and (R⁵)ₙ₅ is geminal difluoro. In some embodiments, n5 is 2 and (R⁵)ₙ₅ is geminal dichloro. In some embodiments, n5 is 2 and two geminal R⁵, together with the carbon atom to which they are attached, form cyclobutane or cyclopentane.

In some embodiments, A is cyclopropane, cyclobutane or cyclopentane; n5 is 2; and (R⁵)ₙ₅ is geminal dimethyl, geminal difluoro or geminal dichloro. In some embodiments, A is cyclopropane, cyclobutane or cyclopentane; n5 is 2; and (R⁵)ₙ₅ is geminal difluoro or geminal dichloro. In some embodiments, A is cyclopropane; n5 is 2 and two geminal R⁵, together with the carbon atom to which they are attached, form cyclobutane or cyclopentane. In some embodiments, A is cyclopropane, cyclobutane, cyclopentane, cyclohexane, and n5 is 0. In some embodiments, A is cyclopropane, cyclobutane or cyclopentane, and n5 is 0. In some embodiments, A is cyclopropane or cyclobutane, and n5 is 0. In some embodiments, A is cyclopropane and n5 is 0.

In some embodiments, each instance of R¹⁰ independently is —F, —Cl, —I, Me, Et, Pr, Bu, iPr, iBu, —OH, —OMe, —OEt, —OPr, —OiPr, —NH₂, —NHMe, —CF₃, —OCF₃, or —CN. In some embodiments, each instance of R¹⁰ independently is —F, —Cl, Me, —OMe, —OEt or —CN. In some embodiments, each instance of R¹⁰ independently is —F, —Cl or —CN. In some embodiments, each instance of R¹⁰ independently is —F, —Cl or Me. In some embodiments, each instance of R¹⁰ independently is —F or —Cl. In some embodiments, each instance of R¹⁰ is —F. In some embodiments, two adjacent R¹⁰ form methylenedioxy, wherein the methylene unit of the methylenedioxy is unsubstituted or substituted with halo.

In some embodiments, n10 is 2 and each instance of R¹⁰ is independently —F, —Cl, —I. In some embodiments, n10 is 2 and R¹⁰ is —F. In some embodiments n10 is 0 or 1, and R¹⁰ is —F, —Cl, —I, Me, —OMe, —OEt or —CN. In some embodiments, n10 is 0. In some embodiments, n10 is 1 and R¹⁰ is —F.

In some embodiments, each of R^{4a} and R^{4b} independently is —H, F, Me, Et, Pr, Bu, iPr, or iBu. In some embodiments, each of R^{4a} and R^{4b} independently is —H, Me, Et, Pr, Bu, iPr, or iBu. In some embodiments, R^{4a} is H and R^{4b} is Me. In some embodiments, R^{4a} is —H. In some embodiments, R^{4b} is —H. In some embodiments, each of R^{4a} and R^{4b} is —H.

In some embodiments, each instance of R⁸ independently is —F, —Cl, Me, Et, Pr, Bu, iPr, iBu, —OH, —OMe, —OEt, —OPr, —OiPr, —NH₂, —NHMe, —NHEt, —NHiPr, —CF₃, —CHF₂ or —CN. In some embodiments, each instance of R⁸ independently is —F, —Cl, Me, —OMe or —OH. In some embodiments, each instance of R⁸ independently is —F, —Cl, Me, or —OMe. In some embodiments, each instance of R⁸ independently is —F, —Cl, or Me. In some embodiments, each instance of R⁸ independently is —F, —Cl, or —OMe. In some embodiments, each instance of R⁸ independently is —F or —Cl. In some embodiments, each instance of R⁸ is —F.

In some embodiments, n8 is 2, and each instance of R⁸ is independently —F or —Cl. In some embodiments, n8 is 0 or 1, and R⁸ is —F, —Cl, Me, —OMe or —OH. In some embodiments, n8 is 1, and R⁸ is —F, —Cl, Me, or —OMe. In some embodiments, n8 is 1, and R⁸ is —F or —Cl. In some embodiments, n8 is 1, and R⁸ is —F. In some embodiments, n8 is 0.

In some embodiments, X¹ is N, and X² and X³ are carbon atoms. In some embodiments, X² is N, and X¹ and X³ are carbon atoms. In some embodiments, X³ is N, and X¹ and X² are carbon atoms.

In some embodiments, X¹ is N, X² and X³ are carbon atoms, and each instance of R⁸ independently is —F, —Cl, Me, —OMe or —OH. In some embodiments, X¹ is N, X² and X³ are carbon atoms, and each instance of R⁸ independently is —F or —Cl. In some embodiments, X² is N, X¹ and X³ are carbon atoms, and each instance of R⁸ independently is —F, —Cl, Me, —OMe or —OH. In some embodiments, X² is N, X¹ and X³ are carbon atoms, and each instance of R⁸ independently is —F or —Cl. In some embodiments, X³ is N, X¹ and X² are carbon atoms, and each instance of R⁸ independently is —F, —Cl, Me, —OMe or —OH. In some embodiments, X³ is N, X¹ and X² are carbon atoms, and each instance of R⁸ independently is —F or —Cl.

In some embodiments, X¹ is N, X² and X³ are carbon atoms, and n8 is 0. In some embodiments, X² is N, X¹ and X³ are carbon atoms, and n8 is 0. In some embodiments, X³ is N, X¹ and X² are carbon atoms, and n8 is 0.

In some embodiments, X¹ is N, each of X² and X³ is CH, n8 is 1, and R⁸ is —F or —Cl. In some embodiments, X² is N, each of X¹ and X³ is CH, n8 is 1, and R⁸ is —F or —Cl. In some embodiments, X³ is N, each of X¹ and X² is CH, n8 is 1, and R⁸ is —F or —Cl.

In some embodiments, Y is —NH— or —N(Me)-. In some embodiments, Y is —NH—. In some embodiments, Y is —N(Me)-.

In some embodiments, A is cyclopropane or cyclobutane; n5 is 0 or 2; (R⁵)ₙ₅ is geminal dimethyl, geminal difluoro or geminal dichloro; n10 is 0, 1, or 2; each instance of R¹⁰ is independently —F or —Cl; each of R^{4a} and R^{4b} is —H; n8 is 0, 1, or 2; each instance of R⁸ is independently is —F or —Cl; and X³ is N, and X¹ and X² are carbon atoms.

In some embodiments, A is cyclopropane or cyclobutane; n5 is 0; n10 is 0, 1, or 2; each instance of R¹⁰ is independently —F or —Cl; each of R^{4a} and R^{4b} is —H; n8 is 0, 1, or 2; each instance of R⁸ is independently is —F or —Cl; and X³ is N, and X¹ and X² are carbon atoms.

In some embodiments (II.B.1′), 1, 2, 3, 4, 5, or 6 instances of —H are replaced with -D (i.e., deuterium, —²H). In some embodiments, 1, 2, 3 or 4 instances of —H are replaced with -D. In some embodiments, at least one instance of -D is present in R^{4a} or R^{4b}. In some embodiments, at least one of R^{4a} and R^{4b} is -D. In some embodiments, R^{4a} is -D. In some embodiments, R^{4b} is -D. In some embodiments, at least one instance of -D is present in R⁵. In some embodiments, at least one instance of -D is present on A. In some embodiments, at least one instance of -D is present in R⁸. In some embodiments, at least one instance of -D is present on the ring to which R⁸ is attached. In some embodiments, at least one instance of -D is present in R¹⁰. In some embodiments, at least one instance of -D is present on the ring to which R¹⁰ is attached.

In some embodiments, a provided chemical entity is a free compound of Formula (II.C.1) or a pharmaceutically acceptable salt thereof, wherein Formula (II.C.1) has the structure,

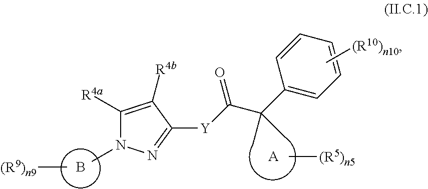

wherein:
A is a C₃₋₆ cycloalkyl or a 3- to 6-membered monocyclic heterocycle containing 1 ring heteroatom selected from O, N and S, wherein the 1 ring heteroatom is not bonded to the carbon to which A is attached;
each instance of R⁵ independently is selected from halo, C₁₋₄ alkyl, C₁₋₄ haloalkyl, —(C(R^{J1a}₂))₀₋₂—OH, —(C(R^{J1a}₂))₀₋₂—OR^{J1}, (C(R^{J1a}₂))₀₋₂—SR^{J1}, —(C(R^{J1a}₂))₀₋₂—NH₂, —(C(R^{J1a}₂))₀₋₂—NHR^{J1}, and —(C(R^{J1a}₂))₀₋₂—NR^{J1}₂, or two geminal R⁵, together with the carbon atom to which they are attached, form a C₄₋₆ cycloalkyl or 4- to 6-membered monocyclic heterocycle containing 1-2 heteroatoms independently selected from O, N, and S,
   wherein each instance of R^{J1} is independently C₁₋₃ alkyl or C₁₋₄ haloalkyl, and
   wherein each instance of R^{J1a} is independently —H, C₁₋₃ alkyl or C₁₋₄ haloalkyl;
n5 is 0, 1 or 2;
B is 5-membered monocyclic heteroaryl having 1-4 ring heteroatoms independently selected from O, N and S, or 6-membered monocyclic heteroaryl having 2 or 3 ring nitrogen atoms;
each instance of R⁹ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, (C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
   wherein each instance of R^{J3} is independently C₁₋₃ alkyl or C₁₋₄ haloalkyl, and
   wherein each instance of R^{J3a} is independently —H, C₁₋₃ alkyl or C₁₋₄ haloalkyl;
n9 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J2a}₂))₀₋₂—SR^{J2}, —(C(R^{J2a}₂))₀₋₂—NH₂, —(C(R^{J2a}₂))₀₋₂—NHR^{J2}, —(C(R^{J2a}₂))₀₋₂—NR^{J2}₂, —C(O)R^{J1}₂, and —CN, or two adjacent R¹⁰ form methylenedioxy, wherein the methylene unit of the methylenedioxy is unsubstituted or substituted with halo;
   wherein each instance of R^{J2} is independently C₁₋₃ alkyl or C₁₋₄ haloalkyl, and
   wherein each instance of R^{J2a} is independently —H, C₁₋₃ alkyl or C₁₋₄ haloalkyl;
n10 is 0, 1, 2 or 3;
each of R^{4a} and R^{4b} independently is —H, halo, or C₁₋₄ alkyl; and
Y is —NH— or —N(C₁₋₄ alkyl)-.

In some embodiments, A is cyclopropane, cyclobutane, cyclopentane, cyclohexane, tetrahydrofuran, tetrahydrothiophene, piperidine or tetrahydropyran, wherein the heteroatom of each of the foregoing applicable rings is not bonded to the carbon to which A is attached. In some embodiments, A is cyclopropane, cyclobutane, cyclopentane or cyclohexane. In some embodiments, A is cyclopropane.

In some embodiments n5 is 0, 1 or 2. In some embodiments, n5 is 0. In some embodiments, n5 is 2 and (R⁵)ₙ₅ is geminal di-(C₁₋₄ alkyl) or geminal di-halo. In some embodiments, n5 is 2 and (R⁵)ₙ₅ is geminal dimethyl. In some embodiments, n5 is 2 and (R⁵)ₙ₅ is geminal difluoro or geminal dichloro.

In some embodiments, A is cyclopropane, cyclobutane or cyclopentane, n5 is 2 and (R⁵)ₙ₅ is geminal difluoro or geminal dichloro. In some embodiments, A is cyclopropane and n5 is 0.

In some embodiments, each instance of R¹⁰ independently is —F, —Cl, Me, —CF₃ or —CN. In some embodiments, each instance of R¹⁰ independently is —F, —Cl or Me. In some embodiments, each instance of R¹⁰ is —F.

In some embodiments n10 is 0 or 1, and R¹⁰ is —F, —Cl, Me, —CF₃ or —CN. In some embodiments, n10 is 0. In some embodiments, n10 is 1 and R¹⁰ is —F.

In some embodiments, R^{4a} is —H. In some embodiments, R^{4b} is —H. In some embodiments, each of R^{4a} and R^{4b} is —H.

In some embodiments, B is pyrazolyl, thiazolyl, isothiazolyl, pyrimidinyl, pyrazinyl, pyridazinyl, or triazinyl. In some embodiments, B is pyrazolyl, thiazolyl, isothiazolyl, pyrimidinyl, pyrazinyl or pyridazinyl. In some embodiments, B is pyrimidinyl, thiazolyl, pyrazinyl or pyridazinyl. In some embodiments, B is pyrimidinyl, pyrazinyl or pyridazinyl. In some embodiments, B is pyrimidinyl or pyridazinyl. In some embodiments, B is pyrimidinyl or thiazolyl. In some embodiments, B is one of the foregoing embodiments and is unsubstituted. In some embodiments, B is one of the foregoing embodiments and is substituted with 1-3 instances of R⁹ as defined herein for Formulas (C), (II.C), and (II.C.1).

In some embodiments, B is pyrimidinyl selected from, an

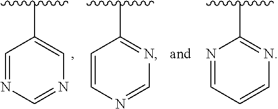

In some embodiments, B is

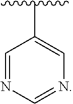 [SMILES: *c1cncnc1]

In some embodiments, B is

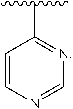 [SMILES: *c1ccncn1]

In some embodiments, B is

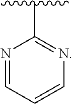 [SMILES: *c1ncccn1]

In some embodiments, B is pyridazinyl selected from

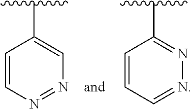 [SMILES: *c1ccnnc1 ; *c1cccnn1]

In some embodiments, B is

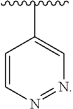 [SMILES: *c1ccnnc1]

In some embodiments, B is

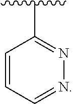 [SMILES: *c1cccnn1]

In some embodiments, B is one of the foregoing embodiments and is unsubstituted. In some embodiments, B is one of the foregoing embodiments and is substituted with 1-3 instances of R⁹ as defined herein for Formulas (C), (II.C), and (II.C.1).

In some embodiments, n9 is 0, 1, or 2 and each instance of R⁹ is independently Me or —OMe. In some embodiments, n9 is 0 or 1, and R⁹ is Me. In some embodiments, n9 is 0 or 1, and R⁹ is Me or —OMe. In some embodiments, n9 is 0. In some embodiments, n9 is 3 and each instance of R⁹ is independently -Me

In some embodiments, B is pyrazolyl, thiazolyl, pyrazinyl or pyridazinyl; n9 is 0 or 1, and R⁹ is Me. In some embodiments, B is pyrimidinyl or thiazolyl, and n9 is 0.

In some embodiments, Y is —NH— or —N(Me)-. In some embodiments, Y is —NH—.

In some embodiments (II.C.1′), 1, 2, 3 or 4 instances of —H are replaced with -D (i.e., deuterium, —²H). In some embodiments, at least one instance of -D is present in R^{4a} or R^{4b}. In some embodiments, at least one of R^{4a} and R^{4b} is -D. In some embodiments, R^{4a} is -D. In some embodiments, R^{4b} is -D. In some embodiments, at least one instance of -D is present in R⁵. In some embodiments, at least one instance of -D is present on A. In some embodiments, at least one instance of -D is present in R⁹. In some embodiments, at least one instance of -D is present on the ring to which R⁹ is attached. In some embodiments, at least one instance of -D is present in R¹⁰. In some embodiments, at least one instance of -D is present on the ring to which R¹⁰ is attached.

In some embodiments, a provided chemical entity is a free compound of Formula (III.A.1) or a pharmaceutically acceptable salt thereof, wherein Formula (III.A.1) has the structure,

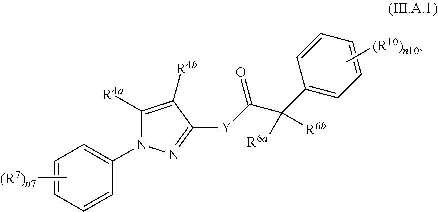

wherein:
each of R^{6a} and R^{6b} independently is —H, C₁₋₄ alkyl, C₁₋₄ haloalkyl, —(C(R^{J1a}₂))₁₋₂—OH, —(C(R^{J1a}₂))₁₋₂—OR^{J1}, —(C(R^{J1a}₂))₁₋₂—SR^{J1}, —(C(R^{J1a}₂))₁₋₂—NH₂, —(C(R^{J1a}₂))₁₋₂—NHR^{J1}, —(C(R^{J1a}₂))₁₋₂—NR^{J1}₂, C₃₋₆ cycloalkyl, or a 3- to 6-membered monocyclic heterocycle containing 1 ring heteroatom selected from O, N, and S,
   wherein the 3- to 6-membered monocyclic heterocycle does not contain a heteroatom bonded to the carbon to which R^{1a} and R^{1b} are attached,
   wherein each instance of R^{J1} is independently C₁₋₃ alkyl or C₁₋₄ haloalkyl, and
   wherein each instance of R^{J1a} is independently —H, C₁₋₃ alkyl or C₁₋₄ haloalkyl;
each instance of R⁷ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, (C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
   wherein each instance of R^{J3} is independently C₁₋₃ alkyl or C₁₋₄ haloalkyl, and
   wherein each instance of R^{J3a} is independently —H, C₁₋₃ alkyl or C₁₋₄ haloalkyl;
n7 is 0, 1, 2 or 3;
each instance of R¹⁰ independently is halo, C₁₋₄ alkyl, C₁₋₄ haloalkyl, —(C(R^{J2a}₂))₀₋₂—OH, —(C(R^{J2a}₂))₀₋₂—OR^{J1}₂, —(C(R^{J2a}₂))₀₋₂—SR^{J2}, —(C(R^{J2a}₂))₀₋₂—NH₂, —(C(R^{J2a}₂))₀₋₂—NHR^{J2}, —(C(R^{J2a}₂))₀₋₂—NR^{J2}₂, —C(O)R^{J1}₂, or —CN,
or two adjacent R¹⁰ form methylenedioxy, wherein the methylene unit of the methylenedioxy is unsubstituted or substituted with halo,
   wherein each instance of R^{J2} is independently C₁₋₃ alkyl or C₁₋₄ haloalkyl, and
   wherein each instance of R^{J2a} is independently —H, C₁₋₃ alkyl or C₁₋₄ haloalkyl;
n10 is 0, 1, 2 or 3;
each of R^{4a} and R^{4b} independently is —H, halo, or C₁₋₄ alkyl; and
Y is —NH— or —N(C₁₋₄ alkyl)-.

In some embodiments, each instance of R¹⁰ independently is Me, Et, Pr, Bu, ⁱPr, ⁱBu, sec-Bu, —F, —Cl, —CF₃, —CHF₂, —OCF₃, —OH, —OMe, —OEt, —OPr, —O-ⁱPr, Ph, —OBn, —NH₂, —NHMe, —NHPr, —SO₂NH₂, —SO₂NHMe, or —CN. In some embodiments, each instance of R¹⁰ independently is Me, ⁱPr, ⁱBu, —F, —Cl, —CF₃, —OCF₃, —OH, —OMe, or —OEt. In some embodiments, each instance of R¹⁰ independently is Me, ⁱPr, ⁱBu, —OH, —OMe, or —OEt. In some embodiments, each instance of R¹⁰ independently is —F, Me, —CF₃, —OMe, or —Cl. In some embodiments, each instance of R¹⁰ independently is —F, Me, —CF₃, or —Cl. In some embodiments, each instance of R¹⁰ independently is —F, Me or —Cl. In some embodiments, each instance of R¹⁰ independently is —F or —Cl. In some embodiments, each instance of R¹⁰ is —F.

In some embodiments, n10 is 0, 1 or 2. In some embodiments, n10 is 2 or 3. In some embodiments, n10 is 2. In some embodiments, n10 is 0 or 1. In some embodiments, n10 is 1. In some embodiments, n10 is 0.

In some embodiments, n10 is 0, 1 or 2, and each instance of R¹⁰ independently is —F, —Cl, Me, or —CF₃. In some embodiments, n10 is 0, 1 or 2, and each instance of R¹⁰ independently is Me, —CF₃, —OMe, —OEt, —OCF₃, iPr, iBu, or —OH. In some embodiments, n10 is 0, 1 or 2, and each instance of R¹⁰ independently is —F or —Cl. In some embodiments, n10 is 0, 1 or 2, and each instance of R¹⁰ independently is —F or Me. In some embodiments, n10 is 1 and R¹⁰ is —F.

In some embodiments, R^{6a} is Me, Et, Pr, Bu, ⁱPr, ⁱBu, sec-Bu, cyclopropyl, cyclobutyl, cyclopentyl, cyclohexyl, or —CF₃, and R^{6b} is —H. In some embodiments, each of R^{6a} and R^{6b} independently is —H, Me, Et or Pr. In some embodiments, R^{6a} is Me, Et, Pr, ⁱPr, cyclopropyl or cyclopentyl. In some embodiments, R^{6a} is Me, Et, iPr or —CF₃, and R^{6b} is Me, Et, Pr, iPr, cyclopropyl, cyclobutyl or cyclopentyl. In some embodiments each of R^{6a} and R^{6b} is —H.

In some embodiments, each of R^{4a} and R^{4b} independently is —H, Me, Et, Pr, Bu, iPr, or iBu. In some embodiments, R^{4a} is —H. In some embodiments R^{4b} is —H. In some embodiments, R^{4a} is —H and R^{4b} is Me. In some embodiments, R^{4a} is Me and R^{4b} is —H. In some embodiments, each of R^{4a} and R^{4b} is —H.

In some embodiments, each instance of R⁷ independently is Me, Et, Pr, Bu, ⁱPr, ⁱBu, sec-Bu, —F, —Cl, —CF₃, —CHF₂, —OCF₃, —OH, —OMe, —OEt, —OPr, —O-iPr, —NH₂, —NHMe, —NHPr, or —CN. In some embodiments, each instance of R⁷ independently is —F, —Cl, —CF₃ or —OH. In some embodiments, each instance of R⁷ independently is —F, —Cl, or —CF₃. In some embodiments, each instance of R⁷ independently is —F or —Cl. In some embodiments, each instance of R⁷ is —F.

In some embodiments, n7 is 0, 1 or 2, and each instance of R⁷ independently is —F, —Cl or —CF₃. In some embodiments, n7 is 0. In some embodiments, n7 is 1 or 2, and each instance of R⁷ independently is —F or —Cl. In some embodiments, n7 is 1 and R⁷ is —F or —Cl. In some embodiments, n7 is 1 and R⁷ is —F.

In some embodiments, Y is —NH— or —N(Me)-. In some embodiments, Y is —NH—.

In some embodiments, R^{4a} is H, R^{4b} is H, Y is —NH—, and n7 is 0. In some embodiments, R^{4a} is H, R^{4b} is H, Y is —NH—, n7 is 1, and R⁷ is —F or —Cl. In some embodiments, R^{4a} is H, R^{4b} is H, Y is —NH—, n7 is 2, and each instance of R⁷ is independently —F or —Cl.

In some embodiments (III.A.1′), 1, 2, 3 or 4 instances of —H are replaced with -D (i.e., deuterium, —²H). In some embodiments, at least one instance of -D is present in R^{4a} or R^{4b}. In some embodiments, at least one of R^{4a} and R^{4b} is -D. In some embodiments, R^{4a} is -D. In some embodiments, R^{4b} is -D. In some embodiments, at least one instance of -D is present in R^{6a} or R^{6b}. In some embodiments, at least one of R^{6a} and R^{6b} is -D. In some embodiments, at least one instance of -D is present in R⁷. In some embodiments, at least one instance of -D is present on the ring to which R⁷ is attached. In some embodiments, at least one instance of -D is present in R¹⁰. In some embodiments, at least one instance of -D is present on the ring to which R¹⁰ is attached.

In some embodiments, a provided chemical entity of Formula (III.A.1) is a chemical entity of Formula (III.A.1a):

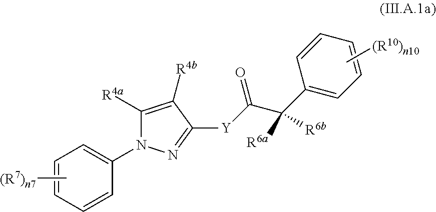

wherein R^{4a}, R^{4b}, R^{6a}, R^{6b}, R⁷, n7, R¹⁰, n10 and Y are as defined above for Formula (III.A.1), both singly and in combination.

In some embodiments, a provided chemical entity of Formula (III.A.1) is a chemical entity of Formula (III.A.1b):

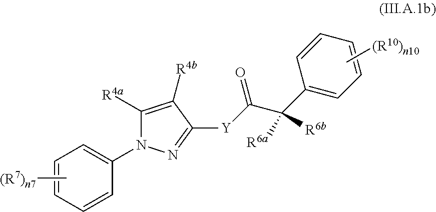

wherein R^{4a}, R^{4b}, R^{6a}, R^{6b}, R⁷, n7, R¹⁰, n10 and Y are as defined above for Formula (III.A.1), both singly and in combination.

In some embodiments, a provided chemical entity is a free compound of Formula (III.A.3) or a pharmaceutically acceptable salt thereof, wherein Formula (III.A.3) has the structure,

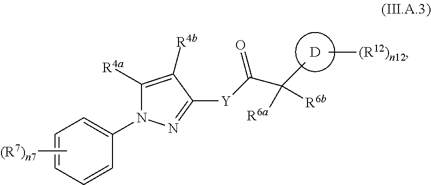

wherein:
each of R^{6a} and R^{6b} independently is —H, C₁₋₄ alkyl, C₁₋₄ haloalkyl, —(C(R^{J1a}₂))₁₋₂—OH, —(C(R^{J1a}₂))₁₋₂—OR^{J1}, —(C(R^{J1a}₂))₁₋₂—SR^{J}, —(C(R^{J1a}₂))₁₋₂—NH₂, —(C(R^{J1a}₂))₁₋₂—NHR^{J1}, —(C(R^{J1a}₂))₁₋₂—NR^{J1}₂, C₃₋₆ cycloalkyl, or a 3- to 6-membered heterocycle containing 1 ring heteroatom selected from O, N, and S,
   wherein the 3- to 6-membered monocyclic heterocycle does not contain a heteroatom bonded to the carbon to which R^{1a} and R^{1b} are attached,
   wherein each instance of R^{J1} is independently C₁₋₃ alkyl or C₁₋₄ haloalkyl, and
   wherein each instance of R^{J1a} is independently —H, C₁₋₃ alkyl or C₁₋₄ haloalkyl;
each instance of R⁷ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂—C(O)R^{J3}, and —CN,
   wherein each instance of R^{J3} is independently C₁₋₃ alkyl or C₁₋₄ haloalkyl, and
   wherein each instance of R^{J3a} is independently —H, C₁₋₃ alkyl or C₁₋₄ haloalkyl;
n7 is 0, 1, 2 or 3;
D is 5- or 6-membered heteroaryl having 1-3 ring heteroatoms independently selected from O, N and S;
each instance of R¹² independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J2a}₂))₀₋₂—SR^{J2}, —(C(R^{J2a}₂))₀₋₂—NH₂, —(C(R^{J2a}₂))₀₋₂—NHR^{J2}, —(C(R^{J2a}₂))₀₋₂—NR^{J2}₂, —C(O)R^{J2}, and —CN,
or two adjacent R¹² form methylenedioxy, wherein the methylene unit of the methylenedioxy is unsubstituted or substituted with halo;
   wherein each instance of R^{J2} is independently C₁₋₃ alkyl or C₁₋₄ haloalkyl, and
   wherein each instance of R^{J2a} is independently —H, C₁₋₃ alkyl or C₁₋₄ haloalkyl;
n12 is 0, 1, 2 or 3;
each of R^{4a} and R^{4b} independently is —H, halo, or C₁₋₄ alkyl; and
Y is —NH— or —N(C₁₋₄ alkyl)-.

In some embodiments, D is thienyl, thiazolyl, pyrimidinyl, pyrazolyl, pyrazinyl or pyridyl. In some embodiments, D is pyrimidinyl or pyridyl.

In some embodiments, n12 is 0 or 1, and R¹² is Me. In some embodiments, n12 is 0.

In some embodiments, D is thienyl, thiazolyl, pyrimidinyl, pyrazolyl, pyrazinyl, or pyridyl; n12 is 0 or 1; and R¹² is Me. In some embodiments, D is pyrimidinyl or pyridyl and n12 is 0.

In some embodiments, R^{4a} is —H. In some embodiments R^{4b} is —H. In some embodiments, each of R^{4a} and R^{4b} is —H.

In some embodiments, each of R^{6a} and R^{6b} is —H.

In some embodiments, n7 is 0 or 1, and R⁷ is —F or —Cl. In some embodiments, n7 is 0.

In some embodiments, Y is —NH— or —N(Me)-. In some embodiments, Y is —NH—.

In some embodiments, each of R^{4a} and R^{4b} is —H, each of R^{6a} and R^{6b} is —H, n7 is 0, and Y is —NH—.

In some embodiments (III.A.3′), 1, 2, 3 or 4 instances of —H are replaced with -D (i.e., deuterium, —²H). In some embodiments, at least one instance of -D is present in R^{4a} or R^{4b}. In some embodiments, at least one of R^{4a} and R^{4b} is -D. In some embodiments, R^{4a} is -D. In some embodiments, R^{4b} is -D. In some embodiments, at least one instance of -D is present in R^{6a} or R^{6b}. In some embodiments, at least one of R^{6a} and R^{6b} is -D. In some embodiments, at least one instance of -D is present in R⁷. In some embodiments, at least one instance of -D is present on the ring to which R⁷ is attached. In some embodiments, at least one instance of -D is present in R¹². In some embodiments, at least one instance of -D is present on the ring to which R¹² is attached.

In some embodiments, a provided chemical entity is a free compound of Formula (III.B.1) or pharmaceutically acceptable salt thereof, wherein Formula (III.B.1) has the structure,

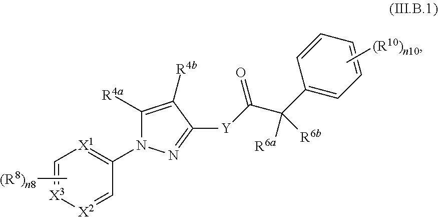

wherein:
each of R^{6a} and R^{6b} independently is —H, C₁₋₄ alkyl, C₁₋₄ haloalkyl, —(C(R^{J1a}₂))₁₋₂—OH, —(C(R^{J1a}₂))₁₋₂—OR^{J1}, —(C(R^{J1a}₂))₁₋₂—SR^{J1}, —(C(R^{J1a}₂))₁₋₂—NH₂, —(C(R^{J1a}₂))₁₋₂—NHR^{J1}, —(C(R^{J1a}₂))₁₋₂—NR^{J1}₂, C₃₋₆ cycloalkyl, or a 3- to 6-membered monocyclic heterocycle containing 1 ring heteroatom selected from O, N, and S,
   wherein the 3- to 6-membered monocyclic heterocycle does not contain a heteroatom bonded to the carbon to which R^{1a} and R^{1b} are attached,
   wherein each instance of R^{J1} is independently C₁₋₃ alkyl or C₁₋₄ haloalkyl, and
   wherein each instance of R^{J1a} is independently —H, C₁₋₃ alkyl or C₁₋₄ haloalkyl;
one of X¹, X² and X³ is N, and the other two are carbon atoms;
each instance of R⁸ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
   wherein each instance of R^{J3} is independently C₁₋₃ alkyl or C₁₋₄ haloalkyl, and
   wherein each instance of R^{J3a} is independently —H, C₁₋₃ alkyl, or C₁₋₄ haloalkyl;
n8 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J2a}₂))₀₋₂—SR^{J2}, —(C(R^{J2a}₂))₀₋₂—NH₂, —(C(R^{J2a}₂))₀₋₂—NHR^{J1}₂, —(C(R^{J2a}₂))₀₋₂—NR^{J2}₂, —C(O)R^{J1}₂, and —CN,
or two adjacent R¹⁰ form methylenedioxy, wherein the methylene unit of the methylenedioxy is unsubstituted or substituted with halo,
   wherein each instance of R^{J2} is independently C₁₋₃ alkyl or C₁₋₄ haloalkyl, and
   wherein each instance of R^{J2a} is independently —H, C₁₋₃ alkyl, or C₁₋₄ haloalkyl;
n10 is 0, 1, 2 or 3;
each of R^{4a} and R^{4b} independently is —H, halo, or C₁₋₄ alkyl; and
Y is —NH— or —N(C₁₋₄ alkyl)-.

In some embodiments, each instance of R¹⁰ independently is —F, —Cl, Me, Et, ⁱPr, —OH, —OMe, —NH₂, —CF₃ or —CN. In some embodiments, each instance of R¹⁰ independently is —F, —Cl, Me, —OMe, —OEt or —CN. In some embodiments, each instance of R¹⁰ independently is —F, —Cl or Me. In some embodiments, each instance of R¹⁰ is —F.

In some embodiments, n10 is 0 or 1, and R¹⁰ is —F, —Cl, Me, —OMe, —OEt or —CN. In some embodiments, n10 is 0. In some embodiments, n10 is 1 and R¹⁰ is —F.

In some embodiments, R^{6a} is Me, Et, Pr, Bu, iPr, iBu, sec-Bu, cyclopropyl, cyclobutyl, cyclopentyl, cyclohexyl, —CF₃, or —OH, and R^{6b} is —H. In some embodiments, each of R^{6a} and R^{6b} independently is —H, Me, Et, Pr, cyclopropyl or cyclopentyl. In some embodiments, R^{6a} is Me, Et, Pr or —CF₃, and R^{6b} is Me, Et, Pr, cyclopropyl or cyclopentyl. In some embodiments, each of R^{6a} and R^{6b} is —H.

In some embodiments, X¹ is N, and X² and X³ are carbon atoms. In some embodiments, X² is N, and X¹ and X³ are carbon atoms. In some embodiments, X³ is N, and X¹ and X² are carbon atoms.

In some embodiments, each instance of R⁸ independently is halo, C₁₋₄ alkyl, C₁₋₄ haloalkyl, —OH, —OMe or —OEt. In some embodiments, each instance of R⁸ independently is —F, —Cl, Me, Et, —CF₃, —OH, —OMe or —OEt. In some embodiments, each instance of R⁸ independently is —F or —Cl.

In some embodiments, X¹ is N, X² and X³ are carbon atoms, and each instance of R⁸ independently is —F, —Cl, Me, Et, —CF₃, —OH, —OMe or —OEt. In some embodiments, X² is N, X¹ and X³ are carbon atoms, and each instance of R⁸ independently is —F, —Cl, Me, Et, —CF₃, —OH, —OMe or —OEt. In some embodiments, X³ is N, X¹ and X² are carbon atoms, and each instance of R⁸ independently is —F, —Cl, Me, Et, —CF₃, —OH, —OMe or —OEt.

In some embodiments, n8 is 0, 1 or 2. In some embodiments, n8 is 0 or 1. In some embodiments, n8 is 1. In some embodiments, n8 is 0.

In some embodiments, n8 is 0 or 1, and R⁸ is —F, —Cl, Me, Et, —CF₃, —OH, —OMe or —OEt. In some embodiments, n8 is 0, 1 or 2, and each instance of R⁸ independently is —F or —Cl.

In some embodiments, Y is —NH— or —N(Me)-. In some embodiments, Y is —NH—.

In some embodiments, n10 is 1, R¹⁰ is —F, each of R^{6a} and R^{6b} is —H, n8 is 1, and R⁸ is —F or —Cl.

In some embodiments (III.B.1′), 1, 2, 3 or 4 instances of —H are replaced with -D (i.e., deuterium, —²H). In some embodiments, at least one instance of -D is present in R^{4a} or R^{4b}. In some embodiments, at least one of R^{4a} and R^{4b} is -D. In some embodiments, R^{4a} is -D. In some embodiments, R^{4b} is -D. In some embodiments, at least one instance of -D is present in R^{6a} or R^{6b}. In some embodiments, at least one of R^{6a} and R^{6b} is -D. In some embodiments, at least one instance of -D is present in R⁸. In some embodiments, at least one instance of -D is present on the ring to which R⁸ is attached. In some embodiments, at least one instance of -D is present in R¹⁰. In some embodiments, at least one instance of -D is present on the ring to which R¹⁰ is attached.

In some embodiments, a provided chemical entity is a free compound of Formula (III.C.1) or a pharmaceutically acceptable salt thereof, wherein Formula (III.C.1) has the structure,

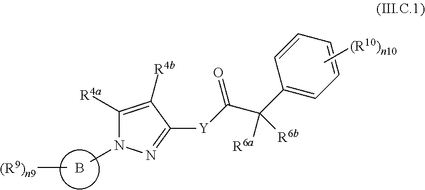

wherein:
each of R^{6a} and R^{6b} independently is —H, C₁₋₄ alkyl, C₁₋₄ haloalkyl or C₃₋₆ cycloalkyl;
B is 5-membered monocyclic heteroaryl having 1-4 ring heteroatoms independently selected from O, N and S, or 6-membered monocyclic heteroaryl having 2 or 3 ring nitrogen atoms;
each instance of R⁹ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
   wherein each instance of R^{J3} is independently C₁₋₃ alkyl or C₁₋₄ haloalkyl, and
   wherein each instance of R^{J3a} is independently —H, C₁₋₃ alkyl or C₁₋₄ haloalkyl;
n9 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J2a}₂))₀₋₂—SR^{J2}, —(C(R^{J2a}₂))₀₋₂—NH₂, —(C(R^{J2a}₂))₀₋₂—NHR^{J2}, —(C(R^{J2a}₂))₀₋₂—NR^{J2}₂, —C(O)R^{J1}₂, and —CN,
or two adjacent R¹⁰ form methylenedioxy, wherein the methylene unit of the methylenedioxy is unsubstituted or substituted with halo,
   wherein each instance of R^{J2} is independently C₁₋₃ alkyl or C₁₋₄ haloalkyl, and
   wherein each instance of R^{J2a} is independently —H, C₁₋₃ alkyl or C₁₋₄ haloalkyl;
n10 is 0, 1, 2 or 3;
each of R^{4a} and R^{4b} independently is —H, halo, or C₁₋₄ alkyl; and
Y is —NH— or —N(C₁₋₄ alkyl)-.

In some embodiments, each instance of R¹⁰ independently is —F, —Cl, Me, Et, —OH, —NH₂ or —CF₃. In some embodiments, each instance of R¹⁰ independently is —F, —Cl, or Me. In some embodiments, each instance of R¹⁰ is —F.

In some embodiments, n10 is 0 or 1, and R¹⁰ is —F, —Cl, Me, Et, —OH, —NH₂ or —CF₃. In some embodiments, n10 is 0. In some embodiments, n10 is 1 and R¹⁰ is —F.

In some embodiments, R^{6a} is Me, Et, cyclopropyl, cyclobutyl, or —CF₃, and R^{6b} is —H. In some embodiments, each of R^{6a} and R^{6b} is —H.

In some embodiments, R^{4a} is —H. In some embodiments R^{4b} is —H. In some embodiments, each of R^{4a} and R^{4b} is —H.

In some embodiments, B is thienyl, thiazolyl, pyrimidinyl, pyrazolyl, pyrazinyl or pyridyl. In some embodiments, B is thiazolyl or pyrimidinyl.

In some embodiments, each instance of R⁹ independently is —F, —Cl, Me, Et, —OH, —NH₂ or —CF₃. In some embodiments, each instance of R⁹ independently is —F, —Cl, or Me. In some embodiments, each instance of R⁹ is Me.

In some embodiments, n9 is 0, 1 or 2, and each instance of R⁹ independently is —F, —Cl, Me, Et, or —CF₃. In some embodiments, n9 is 0. In some embodiments, n9 is 1 or 2, and each instance of R⁹ independently is —F or Me. In some embodiments, n9 is 1 and R⁹ is Me.

In some embodiments, Y is —NH— or —N(Me)-. In some embodiments, Y is —NH—.

In some embodiments, n10 is 1 and R¹⁰ is —F or —Cl, each of R^{6a} and R^{6b} is —H, each of R^{4a} and R^{4b} is —H, B is thiazolyl or pyrimidinyl, n9 is 0 or 1, and R⁹ is Me.

In some embodiments (III.C.1′), 1, 2, 3 or 4 instances of —H are replaced with -D (i.e., deuterium, —²H). In some embodiments, at least one instance of -D is present in R^{4a} or R^{4b}. In some embodiments, at least one of R^{4a} and R^{4b} is -D. In some embodiments, R^{4a} is -D. In some embodiments, R^{4b} is -D. In some embodiments, at least one instance of -D is present in R^{6a} or R^{6b}. In some embodiments, at least one of R^{6a} and R^{6b} is -D. In some embodiments, at least one instance of -D is present in R⁹. In some embodiments, at least one instance of -D is present on B. In some embodiments, at least one instance of -D is present in R¹⁰. In some embodiments, at least one instance of -D is present on the ring to which R¹⁰ is attached.

In some embodiments, a provided chemical entity is a free compound from Table 1 or a pharmaceutically acceptable salt thereof. In some embodiments, a provided chemical entity is a free compound from Table 1. In some embodiments, a provided chemical entity is a pharmaceutically acceptable salt of a free compound from Table 1.

TABLE 1
Compound Names (IUPAC Nomenclature)

| Compound | |
|---|---|
| Number | IUPAC chemical name* |
| 1 | 2-(2-fluorophenyl)-N-(1-phenylpyrazol-3-yl)acetamide |
| 2 | 1-phenyl-N-(1-phenylpyrazol-3-yl)cyclopropanecarboxamide |
| 3 | 2-(2-fluorophenyl)-N-(4-methyl-1-phenyl-pyrazol-3-yl)acetamide |
| 4 | N-[1-(2-chloro-4-pyridyl)pyrazol-3-yl]-2-(2-fluorophenyl)acetamide |
| 5 | N-(1-phenylpyrazol-3-yl)-2-(3-thienyl)acetamide |
| 6 | 2-(4-chlorophenyl)-N-(1-phenylpyrazol-3-yl)acetamide |
| 7 | 2-(2,3-dimethoxyphenyl)-N-(1-phenylpyrazol-3-yl)acetamide |
| 8 | 2-(4-hydroxyphenyl)-N-(1-phenylpyrazol-3-yl)acetamide |
| 9 | 2-(4-hydroxy-3-methoxy-phenyl)-N-(1-phenylpyrazol-3-yl)acetamide |
| 10 | 2-(4-isopropylphenyl)-N-(1-phenylpyrazol-3-yl)acetamide |
| 11 | 2-[4-fluoro-2-(trifluoromethyl)phenyl]-N-(1-phenylpyrazol-3-yl)acetamide |
| 12 | 1-(o-tolyl)-N-(1-phenylpyrazol-3-yl)cyclopropanecarboxamide |
| 13 | 1-(4-fluorophenyl)-N-(1-phenylpyrazol-3-yl)cyclopropanecarboxamide |
| 14 | 2-(2-fluorophenyl)-N-[1-(4-pyridyl)pyrazol-3-yl]acetamide |
| 15 | 2-(2-fluorophenyl)-N-(1-pyrimidin-5-ylpyrazol-3-yl)acetamide |
| 16 | 2-(4-fluorophenyl)-N-(1-phenylpyrazol-3-yl)acetamide |
| 17 | N-(1-phenylpyrazol-3-yl)-2-(3,4,5-trifluorophenyl)acetamide |
| 18 | 2-(2,4-difluorophenyl)-N-(1-phenylpyrazol-3-yl)acetamide |
| 19 | 1-(3-chlorophenyl)-N-(1-phenylpyrazol-3-yl)cyclopropanecarboxamide |
| 20 | (2S)-2-phenyl-N-(1-phenylpyrazol-3-yl)propanamide |
| 21 | 1-(3-fluorophenyl)-N-(1-phenylpyrazol-3-yl)cyclopropanecarboxamide |
| 22 | 1-(2-fluorophenyl)-N-(1-phenylpyrazol-3-yl)cyclopropanecarboxamide |
| 23 | 1-(4-chlorophenyl)-N-(1-phenylpyrazol-3-yl)cyclopropanecarboxamide |
| 24 | 1-(2-chlorophenyl)-N-(1-phenylpyrazol-3-yl)cyclopropanecarboxamide |
| 25 | 1-(4-methoxyphenyl)-N-(1-phenylpyrazol-3-yl)cyclopropanecarboxamide |
| 26 | 1-(3-methoxyphenyl)-N-(1-phenylpyrazol-3-yl)cyclopropanecarboxamide |
| 27 | (2R)-2-phenyl-N-(1-phenylpyrazol-3-yl)propanamide |
| 28 | (2R)-2-phenyl-N-(1-phenylpyrazol-3-yl)butanamide |
| 29 | 1-[3-fluoro-5-(trifluoromethyl)phenyl]-N-(1-phenylpyrazol-3-yl)cyclopropanecarboxamide |
| 30 | 2-methyl-2-phenyl-N-(1-phenylpyrazol-3-yl)propanamide |
| 31 | 3-methyl-2-phenyl-N-(1-phenylpyrazol-3-yl)butanamide |
| 32 | 1-(m-tolyl)-N-(1-phenylpyrazol-3-yl)cyclopropanecarboxamide |
| 33 | 1-(4-ethoxyphenyl)-N-(1-phenylpyrazol-3-yl)cyclopropanecarboxamide |
| 34 | 1-phenyl-N-(1-phenylpyrazol-3-yl)cyclopentanecarboxamide |
| 35 | 1-phenyl-N-(1-phenylpyrazol-3-yl)cyclobutanecarboxamide |
| 36 | 1-(3,5-dimethylphenyl)-N-(1-phenylpyrazol-3-yl)cyclopropanecarboxamide |
| 37 | N-(1-phenylpyrazol-3-yl)-2-(2-thienyl)acetamide |
| 38 | N-(1-phenylpyrazol-3-yl)-2-(4-pyridyl)acetamide |
| 39 | N-(1-phenylpyrazol-3-yl)-2-(3-pyridyl)acetamide |
| 40 | N-(1-phenylpyrazol-3-yl)-2-(2-pyridyl)acetamide |
| 41 | 2-phenyl-N-(1-phenylpyrazol-3-yl)acetamide |
| 42 | N-(1-phenylpyrazol-3-yl)-2-(p-tolyl)acetamide |
| 43 | 2-(m-tolyl)-N-(1-phenylpyrazol-3-yl)acetamide |
| 44 | 2-(o-tolyl)-N-(1-phenylpyrazol-3-yl)acetamide |
| 45 | 2-(4-methoxyphenyl)-N-(1-phenylpyrazol-3-yl)acetamide |
| 46 | 2-(3-methoxyphenyl)-N-(1-phenylpyrazol-3-yl)acetamide |
| 47 | 2-(4-ethoxyphenyl)-N-(1-phenylpyrazol-3-yl)acetamide |
| 48 | 2-(2-ethoxyphenyl)-N-(1-phenylpyrazol-3-yl)acetamide |
| 49 | 2-(3-fluorophenyl)-N-(1-phenylpyrazol-3-yl)acetamide |
| 50 | 2-(3-chlorophenyl)-N-(1-phenylpyrazol-3-yl)acetamide |
| 51 | N-(1-phenylpyrazol-3-yl)-2-[3-(trifluoromethyl)phenyl]acetamide |
| 52 | N-(1-phenylpyrazol-3-yl)-2-[2-(trifluoromethyl)phenyl]acetamide |
| 53 | N-(1-phenylpyrazol-3-yl)-2-[4-(trifluoromethoxy)phenyl]acetamide |
| 54 | N-(1-phenylpyrazol-3-yl)-2-[2-(trifluoromethoxy)phenyl]acetamide |
| 55 | 2-(2,5-dimethylphenyl)-N-(1-phenylpyrazol-3-yl)acetamide |
| 56 | 2-(3-fluoro-4-hydroxy-phenyl)-N-(1-phenylpyrazol-3-yl)acetamide |
| 57 | 2-(3,4-difluorophenyl)-N-(1-phenylpyrazol-3-yl)acetamide |
| 58 | 2-(3,5-difluorophenyl)-N-(1-phenylpyrazol-3-yl)acetamide |
| 59 | 2-(2,5-difluorophenyl)-N-(1-phenylpyrazol-3-yl)acetamide |
| 60 | 2-(2,3-difluorophenyl)-N-(1-phenylpyrazol-3-yl)acetamide |
| 61 | 2-(2,6-difluorophenyl)-N-(1-phenylpyrazol-3-yl)acetamide |
| 62 | 2-(4-methoxy-3-methyl-phenyl)-N-(1-phenylpyrazol-3-yl)acetamide |
| 63 | 2-(3-fluoro-4-methoxy-phenyl)-N-(1-phenylpyrazol-3-yl)acetamide |
| 64 | 2-(3-chloro-4-fluoro-phenyl)-N-(1-phenylpyrazol-3-yl)acetamide |
| 65 | 2-(4-chloro-2-fluoro-phenyl)-N-(1-phenylpyrazol-3-yl)acetamide |
| 66 | 2-(3,4-dimethoxylphenyl)-N-(1-phenylpyrazol-3-yl)acetamide |
| 67 | 2-(2,5-dimethoxyphenyl)-N-(1-phenylpyrazol-3-yl)acetamide |
| 68 | 2-(3,4-dichlorophenyl)-N-(1-phenylpyrazol-3-yl)acetamide |
| 69 | 2-(2,3-dichlorophenyl)-N-(1-phenylpyrazol-3-yl)acetamide |
| 70 | 2-(2,6-dichlorophenyl)-N-(1-phenylpyrazol-3-yl)acetamide |
| 71 | 2-[4-fluoro-3-(trifluoromethyl)phenyl]-N-(1-phenylpyrazol-3-yl)acetamide |
| 72 | 2-[3-fluoro-5-(trifluoromethyl)phenyl]-N-(1-phenylpyrazol-3-yl)acetamide |
| 73 | 2-[3,5-bis(trifluoromethyl)phenyl]-N-(1-phenylpyrazol-3-yl)acetamide |
| 74 | N-(1-phenylpyrazol-3-yl)-2-(2,3,4-trifluorophenyl)acetamide |
| 75 | N-(1-phenylpyrazol-3-yl)-2-(2,4,5-trifluorophenyl)acetamide |
| 76 | N-(1-phenylpyrazol-3-yl)-2-(2,4,6-trifluorophenyl)acetamide |
| 77 | N-(1-phenylpyrazol-3-yl)-2-(p-tolyl)propanamide |
| 78 | 2-(4-isobutylphenyl)-N-(1-phenylpyrazol-3-yl)propanamide |
| 79 | 1-(4-chlorophenyl)-N-(1-phenylpyrazol-3-yl)cyclopentanecarboxamide |
| 80 | 2-phenyl-N-(1-phenylpyrazol-3-yl)propanamide |
| 81 | 1-phenyl-N-(1-thiazol-2-ylpyrazol-3-yl)cyclopropanecarboxamide |
| 82 | N-[1-(3-fluoro-2-pyridyl)pyrazol-3-yl]-1-phenyl-cyclopropanecarboxamide |
| 83 | N-[1-(2-chloro-4-pyridyl)pyrazol-3-yl]-2-phenyl-acetamide |
| 84 | N-[1-(2-chloro-4-pyridyl)pyrazol-3-yl]-1-phenyl-cyclopropanecarboxamide |
| 85 | 1-(2-fluorophenyl)-N-(1-pyrimidin-5-ylpyrazol-3-yl)cyclopropanecarboxamide |
| 86 | 2-phenyl-N-[1-(2-pyridyl)pyrazol-3-yl]acetamide |
| 87 | 1-(2-fluorophenyl)-N-[1-(2-fluoro-4-pyridyl)pyrazol-3-yl]cyclopropanecarboxamide |
| 88 | 2-(4-fluorophenyl)-N-[1-(3-fluoro-2-pyridyl)pyrazol-3-yl]acetamide |
| 89 | 2-phenyl-N-(1-thiazol-2-ylpyrazol-3-yl)acetamide |
| 90 | 2-(2-fluorophenyl)-N-(1-thiazol-2-ylpyrazol-3-yl)acetamide |
| 91 | 2-(4-fluorophenyl)-N-(1-thiazol-2-ylpyrazol-3-yl)acetamide |
| 92 | 1-(2-fluorophenyl)-N-(1-thiazol-2-ylpyrazol-3-yl)cyclopropanecarboxamide |
| 93 | N-[1-(3-fluoro-2-pyridyl)pyrazol-3-yl]-2-phenyl-acetamide |
| 94 | 2-(2-fluorophenyl)-N-[1-(3-fluoro-2-pyridyl)pyrazol-3-yl]acetamide |
| 95 | 1-(2-fluorophenyl)-N-[1-(3-fluoro-2-pyridyl)pyrazol-3-yl]cyclopropanecarboxamide |
| 96 | 2-phenyl-N-(1-pyrimidin-5-ylpyrazol-3-yl)acetamide |
| 97 | 2-(4-fluorophenyl)-N-(1-pyrimidin-5-ylpyrazol-3-yl)acetamide |
| 98 | 1-phenyl-N-(1-pyrimidin-5-ylpyrazol-3-yl)cyclopropanecarboxamide |
| 99 | 2-phenyl-N-[1-(4-pyridyl)pyrazol-3-yl]acetamide |
| 100 | 1-phenyl-N-[1-(4-pyridyl)pyrazol-3-yl]cyclopropanecarboxamide |
| 101 | 1-(2-fluorophenyl)-N-[1-(4-pyridyl)pyrazol-3-yl]cyclopropanecarboxamide |
| 102 | 2-(4-fluorophenyl)-N-[1-(4-pyridyl)pyrazol-3-y]+acetamide |
| 103 | N-[1-(6-chloro-3-pyridyl)pyrazol-3-yl]-2-phenyl-acetamide |
| 104 | N-[1-(6-chloro-3-pyridyl)pyrazol-3-yl]-2-(2-fluorophenyl)acetamide |
| 105 | N-[1-(6-chloro-3-pyridyl)pyrazol-3-yl]-2-(4-fluorophenyl)acetamide |
| 106 | N-[1-(6-chloro-3-pyridyl)pyrazol-3-yl]-1-phenyl-cyclopropanecarboxamide |
| 107 | N-[1-(6-chloro-3-pyridyl)pyrazol-3-yl]-1-(2-fluorophenyl)cyclopropanecarboxamide |
| 108 | 1-(4-fluorophenyl)-N-[1-(2-methyl-4-pyridyl)pyrazol-3-yl]cyclopropanecarboxamide |
| 109 | N-[1-(2-methyl-4-pyridyl)pyrazol-3-yl]-2-phenyl-acetamide |
| 110 | 2-(2-fluorophenyl)-N-[1-(2-methyl-4-pyridyl)pyrazol-3-yl]acetamide |
| 111 | 2-(4-fluorophenyl)-N-[1-(2-methyl-4-pyridyl)pyrazol-3-yl]acetamide |
| 112 | N-[1-(2-methyl-4-pyridyl)pyrazol-3-yl]-1-phenyl-cyclopropanecarboxamide |
| 113 | 1-(2-fluorophenyl)-N-[1-(2-methyl-4-pyridyl)pyrazol-3-yl]cyclopropanecarboxamide |
| 114 | N-[1-(2,5-difluoro-4-pyridyl)pyrazol-3-yl]-2-phenyl-acetamide |
| 115 | N-[1-(2,5-difluoro-4-pyridyl)pyrazol-3-yl]-1-phenyl-cyclopropanecarboxamide |
| 116 | N-[1-(2,5-difluoro-4-pyridyl)pyrazol-3-yl]-1-(2-fluorophenyl)cyclopropanecarboxamide |
| 117 | N-[1-(6-methyl-3-pyridyl)pyrazol-3-yl]-2-phenyl-acetamide |
| 118 | N-[1-(6-methyl-3-pyridyl)pyrazol-3-yl]-1-phenyl-cyclopropanecarboxamide |
| 119 | 2-(2-fluorophenyl)-N-[1-(6-methyl-3-pyridyl)pyrazol-3-yl]acetamide |
| 120 | 1-(2-fluorophenyl)-N-[1-(6-methyl-3-pyridyl)pyrazol-3-yl]cyclopropanecarboxamide |
| 121 | N-[1-(3-chlorophenyl)pyrazol-3-yl]-2-phenyl-acetamide |
| 122 | 2-(2-fluorophenyl)-N-[1-[2-(trifluoromethyl)-4-pyridyl]pyrazol-3-yl]acetamide |
| 123 | 2-phenyl-N-[1-[2-(trifluoromethyl)-4-pyridyl]pyrazol-3-yl]acetamide |
| 124 | 1-(2-fluorophenyl)-N-[1-(3-fluorophenyl)pyrazol-3-yl]cyclopropanecarboxamide |
| 125 | N-[1-(3-fluorophenyl)pyrazol-3-yl]-1-phenyl-cyclopropanecarboxamide |
| 126 | 2-(2-fluorophenyl)-N-[1-(3-fluorophenyl)pyrazol-3-yl]acetamide |
| 127 | N-[1-(3-fluorophenyl)pyrazol-3-yl]-2-phenyl-acetamide |
| 128 | N-[1-(3-chlorophenyl)pyrazol-3-yl]-1-(2-fluorophenyl)cyclopropanecarboxamide |
| 129 | N-[1-(3-chlorophenyl)pyrazol-3-yl]-1-phenyl-cyclopropanecarboxamide |
| 130 | N-[1-(3-chlorophenyl)pyrazol-3-yl]-2-(2-fluorophenyl)acetamide |
| 131 | N-[1-(4-chlorophenyl)pyrazol-3-yl]-1-(2-fluorophenyl)cyclopropanecarboxamide |
| 132 | N-[1-(4-chlorophenyl)pyrazol-3-yl]-1-phenyl-cyclopropanecarboxamide |
| 133 | N-[1-(4-chlorophenyl)pyrazol-3-yl]-2-(2-fluorophenyl)acetamide |
| 134 | N-[1-(4-chlorophenyl)pyrazol-3-yl]-2-phenyl-acetamide |
| 135 | 1-(2-fluorophenyl)-2,2-dimethyl-N-(1-phenylpyrazol-3-yl)cyclopropanecarboxamide |
| 136 | 2,2-dimethyl-1-phenyl-N-(1-phenylpyrazol-3-yl)cyclopropanecarboxamide |
| 137 | 1-(2-fluorophenyl)-N-[1-(3-fluoro-4-pyridyl)pyrazol-3-yl]cyclopropanecarboxamide |
| 138 | N-[1-(3-fluoro-4-pyridyl)pyrazol-3-yl]-1-phenyl-cyclopropanecarboxamide |
| 139 | 2-(2-fluorophenyl)-N-[1-(3-fluoro-4-pyridyl)pyrazol-3-yl]acetamide |
| 140 | N-[1-(3-fluoro-4-pyridyl)pyrazol-3-yl]-2-phenyl-acetamide |
| 141 | 2,2-dichloro-1-phenyl-N-(1-phenylpyrazol-3-yl)cyclopropanecarboxamide |
| 142 | 1-phenyl-N-(1-phenylpyrazol-3-yl)cyclohexanecarboxamide |
| 143 | 2,2-difluoro-1-phenyl-N-(1-phenylpyrazol-3-yl)cyclopropanecarboxamide |
| 144 | 3-phenyl-N-(1-phenylpyrazol-3-yl)oxetane-3-carboxamide |
| 145 | (2R)-2-phenyl-N-(1-phenylpyrazol-3-yl)spiro[2.4]heptane-2-carboxamide |
| 146 | (2S)-2-phenyl-N-(1-phenylpyrazol-3-yl)spiro[2.3]hexane-2-carboxamide |
| 147 | 2,2-dichloro-N-[1-(2-fluoro-4-pyridyl)pyrazol-3-yl]-1-phenyl-cyclopropanecarboxamide |
| 148 | N-[1-(2-fluoro-4-pyridyl)pyrazol-3-yl]-1-phenyl-cyclohexanecarboxamide |
| 149 | N-[1-(2-fluoro-4-pyridyl)pyrazol-3-yl]-3-phenyl-oxetane-3-carboxamide |
| 150 | (2S)-N-[1-(2-fluoro-4-pyridyl)pyrazol-3-yl]-2-phenyl-spiro[2.4]heptane-2-carboxamide |
| 151 | (2S)-N-[1-(2-fluoro-4-pyridyl)pyrazol-3-yl]-2-phenyl-spiro[2.3]hexane-2-carboxamide |
| 152 | 2-cyclopentyl-N-[1-(2-fluoro-4-pyridyl)pyrazol-3-yl]-2-phenyl-acetamide |
| 153 | 2-phenyl-N-[1-[4-(trifluoromethyl)phenyl]pyrazol-3-yl]acetamide |
| 154 | 1-(2-fluorophenyl)-N-[1-[4-(trifluoromethyl)phenyl]pyrazol-3-yl]cyclopropanecarboxamide |
| 155 | 2-(2-fluorophenyl)-N-[1-[4-(trifluoromethyl)phenyl]pyrazol-3-yl]acetamide |
| 156 | 1-phenyl-N-[1-[4-(trifluoromethyl)phenyl]pyrazol-3-yl]cyclopropanecarboxamide |
| 157 | 2-phenyl-N-[1-[3-(trifluoromethyl)phenyl]pyrazol-3-yl]acetamide |
| 158 | 2-(2-fluorophenyl)-N-[1-[3-(trifluoromethyl)phenyl]pyrazol-3-yl]acetamide |
| 159 | 1-phenyl-N-[1-[3-(trifluoromethyl)phenyl]pyrazol-3-yl]cyclopropanecarboxamide |
| 160 | N-[1-(2,5-difluorophenyl)pyrazol-3-yl]-2-phenyl-acetamide |
| 161 | N-[1-(2,5-difluorophenyl)pyrazol-3-yl]-1-(2-fluorophenyl)cyclopropanecarboxamide |
| 162 | N-[1-(2,5-difluorophenyl)pyrazol-3-yl]-2-(2-fluorophenyl)acetamide |
| 163 | N-[1-(2,5-difluorophenyl)pyrazol-3-yl]-1-phenyl-cyclopropanecarboxamide |
| 164 | (2R)-N-[1-(2-fluoro-4-pyridyl)pyrazol-3-yl]-2-phenyl-spiro[2.4]heptane-2-carboxamide |
| 165 | N-[1-(2-fluoro-4-pyridyl)pyrazol-3-yl]-2,2-dimethyl-1-phenyl-cyclopropanecarboxamide |
| 166 | 2-ethyl-2-methyl-1-phenyl-N-(1-phenylpyrazol-3-yl)cyclopropanecarboxamide |
| 167 | 2-cyclopentyl-2-phenyl-N-(1-phenylpyrazol-3-yl)acetamide |
| 168 | 2-ethyl-N-[1-(2-fluoro-4-pyridyl)pyrazol-3-yl]-2-methyl-1-phenyl-cyclopropanecarboxamide |
| 169 | 2,2-difluoro-N-[1-(2-fluoro-4-pyridyl)pyrazol-3-yl]-1-phenyl-cyclopropanecarboxamide |
| 170 | 3-methylsulfanyl-2-phenyl-N-(1-phenylpyrazol-3-yl)propanamide |
| 171 | N-[1-(2-fluoro-4-pyridyl)pyrazol-3-yl]-2-phenyl-butanamide |
| 172 | 3,3-difluoro-N-[1-(2-fluoro-4-pyridyl)pyrazol-3-yl]-1-phenyl-cyclobutanecarboxamide |
| 173 | N-[1-(2-fluoro-4-pyridyl)pyrazol-3-yl]-2-phenyl-pentanamide |
| 174 | N-[1-(2-fluoro-4-pyridyl)pyrazol-3-yl]-4-phenyl-tetrahydropyran-4-carboxamide |
| 175 | 2-cyclopropyl-N-[1-(2-fluoro-4-pyridyl)pyrazol-3-yl]-2-phenyl-acetamide |
| 176 | 2-phenyl-N-(1-phenylpyrazol-3-yl)pentanamide |
| 177 | 3,3-difluoro-1-phenyl-N-(1-phenylpyrazol-3-yl)cyclobutanecarboxamide |
| 178 | 4-phenyl-N-(1-phenylpyrazol-3-yl)tetrahydropyran-4-carboxamide |
| 179 | 2-cyclopropyl-2-phenyl-N-(1-phenylpyrazol-3-yl)acetamide |
| 180 | 1-(2-fluorophenyl)-N-[1-(5-fluoro-3-pyridyl)pyrazol-3-yl]cyclopropanecarboxamide |
| 181 | N-[1-(4-fluorophenyl)pyrazol-3-yl]-2-phenyl-acetamide |
| 182 | 1-(2-fluorophenyl)-N-[1-(4-fluorophenyl)pyrazol-3-yl]cyclopropanecarboxamide |
| 183 | 2-(2-fluorophenyl)-N-[1-(4-fluorophenyl)pyrazol-3-yl]acetamide |
| 184 | N-[1-(4-fluorophenyl)pyrazol-3-yl]-1-phenyl-cyclopropanecarboxamide |
| 185 | N-[1-(2-fluorophenyl)pyrazol-3-yl]-2-phenyl-acetamide |
| 186 | 1-(2-fluorophenyl)-N-[1-(2-fluorophenyl)pyrazol-3-yl]cyclopropanecarboxamide |
| 187 | 2-(2-fluorophenyl)-N-[1-(2-fluorophenyl)pyrazol-3-yl]acetamide |
| 188 | N-[1-(2-fluorophenyl)pyrazol-3-yl]-1-phenyl-cyclopropanecarboxamide |
| 189 | N-[1-(3,4-difluorophenyl)pyrazol-3-yl]-2-phenyl-acetamide |
| 190 | N-[1-(3,4-difluorophenyl)pyrazol-3-yl]-1-(2-fluorophenyl)cyclopropanecarboxamide |
| 191 | N-[1-(3,4-difluorophenyl)pyrazol-3-yl]-2-(2-fluorophenyl)acetamide |
| 192 | N-[1-(3,4-difluorophenyl)pyrazol-3-yl]-1-phenyl-cyclopropanecarboxamide |
| 193 | N-[1-(4-chloro-3-fluoro-phenyl)pyrazol-3-yl]-2-phenyl-acetamide |
| 194 | N-[1-(4-chloro-3-fluoro-phenyl)pyrazol-3-yl]-1-(2-fluorophenyl)cyclopropanecarboxamide |
| 195 | N-[1-(4-chloro-3-fluoro-phenyl)pyrazol-3-yl]-2-(2-fluorophenyl)acetamide |
| 196 | N-[1-(4-chloro-3-fluoro-phenyl)pyrazol-3-yl]-1-phenyl-cyclopropanecarboxamide |
| 197 | N-[1-(3-chloro-4-fluoro-phenyl)pyrazol-3-yl]-2-phenyl-acetamide |
| 198 | N-[1-(3-chloro-4-fluoro-phenyl)pyrazol-3-yl]-1-(2-fluorophenyl)cyclopropanecarboxamide |
| 199 | N-[1-(3-chloro-4-fluoro-phenyl)pyrazol-3-yl]-2-(2-fluorophenyl)acetamide |
| 200 | N-[1-(3-chloro-4-fluoro-phenyl)pyrazol-3-yl]-1-phenyl-cyclopropanecarboxamide |
| 201 | N-[1-(5-fluoro-3-pyridyl)pyrazol-3-yl]-1-phenyl-cyclopropanecarboxamide |
| 202 | 1-(2-fluorophenyl)-N-(1-pyridazin-4-ylpyrazol-3-yl)cyclopropanecarboxamide |
| 203 | 2-(2-fluorophenyl)-N-[1-(2-fluoro-4-pyridyl)pyrazol-3-yl]acetamide |
| 204 | 2-(2-fluorophenyl)-N-[1-(5-fluoro-3-pyridyl)pyrazol-3-yl]acetamide |
| 205 | N-[1-(5-fluoro-3-pyridyl)pyrazol-3-yl]-2-phenyl-acetamide |
| 206 | 1-(2-fluorophenyl)-N-(1-pyrimidin-4-ylpyrazol-3-yl)cyclopropanecarboxamide |
| 207 | 1-phenyl-N-(1-pyrimidin-4-ylpyrazol-3-yl)cyclopropanecarboxamide |
| 208 | 2-(2-fluorophenyl)-N-(1-pyrimidin-4-ylpyrazol-3-yl)acetamide |
| 209 | 2-phenyl-N-(1-pyrimidin-4-ylpyrazol-3-yl)acetamide |
| 210 | 1-(2-fluorophenyl)-N-[1-(2-pyridyl)pyrazol-3-yl]cyclopropanecarboxamide |
| 211 | 2-(2-fluorophenyl)-N-[1-(2-pyridyl)pyrazol-3-yl]acetamide |
| 212 | 1-phenyl-N-[1-(2-pyridyl)pyrazol-3-yl]cyclopropanecarboxamide |
| 213 | 2-phenyl-N-[1-(3-pyridyl)pyrazol-3-yl]acetamide |
| 214 | 1-(2-fluorophenyl)-N-[1-(3-pyridyl)pyrazol-3-yl]cyclopropanecarboxamide |
| 215 | 1-(2-fluorophenyl)-N-(1-pyrazin-2-ylpyrazol-3-yl)cyclopropanecarboxamide |
| 216 | 2-(2-fluorophenyl)-N-(1-pyrazin-2-ylpyrazol-3-yl)acetamide |
| 217 | 1-(2-fluorophenyl)-N-(1-thiazol-5-ylpyrazol-3-yl)cyclopropanecarboxamide |
| 218 | 2-phenyl-N-(1-pyrazin-2-ylpyrazol-3-yl)acetamide |
| 219 | N-(1-phenylpyrazol-3-yl)-2-pyrazin-2-yl-acetamide |
| 220 | N-(1-phenylpyrazol-3-yl)-2-pyrimidin-2-yl-acetamide |
| 221 | N-[1-(2-fluoro-4-pyridyl)pyrazol-3-yl]-2-pyrimidin-2-yl-acetamide |
| 222 | N-[1-(2-fluoro-4-pyridyl)pyrazol-3-yl]-1-(3-pyridyl)cyclopropanecarboxamide |
| 223 | N-[1-(2-fluoro-4-pyridyl)pyrazol-3-yl]-1-(2-pyridyl)cyclopropanecarboxamide |
| 224 | N-(1-phenylpyrazol-3-yl)-1-(3-pyridyl)cyclopropanecarboxamide |
| 225 | N-(1-phenylpyrazol-3-yl)-1-(2-pyridyl)cyclopropanecarboxamide |
| 226 | 1-(4-cyanophenyl)-N-[1-(2-fluoro-4-pyridyl)pyrazol-3-yl]cyclopropanecarboxamide |
| 227 | 1-(4-cyanophenyl)-N-(1-phenylpyrazol-3-yl)cyclopropanecarboxamide |
| 228 | N-[1-(1-methylpyrazol-3-yl)pyrazol-3-yl]-2-phenyl-acetamide |
| 229 | 1-(2-fluorophenyl)-N-[1-(1-methylpyrazol-3-yl)pyrazol-3-yl]cyclopropanecarboxamide |
| 230 | 2-(2-fluorophenyl)-N-[1-(1-methylpyrazol-3-yl)pyrazol-3-yl]acetamide |
| 231 | N-[1-(1-methylpyrazol-3-yl)pyrazol-3-yl]-1-phenyl-cyclopropanecarboxamide |
| 232 | N-[1-(2-methoxy-4-pyridyl)pyrazol-3-yl]-2-phenyl-acetamide |
| 233 | 1-phenyl-N-(1-pyrazin-2-ylpyrazol-3-yl)cyclopropanecarboxamide |
| 234 | 1-phenyl-N-[1-(3-pyridyl)pyrazol-3-yl]cyclopropanecarboxamide |
| 235 | 2-(2-fluorophenyl)-N-[1-(3-pyridyl)pyrazol-3-yl]acetamide |
| 236 | N-[1-(2-fluoro-4-pyridyl)pyrazol-3-yl]-2-pyrimidin-5-yl-acetamide |
| 237 | N-[1-(2-fluoro-4-pyridyl)pyrazol-3-yl]-1-phenyl-cyclopropanecarboxamide |
| 238 | N-[1-(2-fluoro-4-pyridyl)pyrazol-3-yl]-1-(4-iodophenyl)cyclopropanecarboxamide |
| 239 | 1-(2-fluorophenyl)-N-(1-pyridazin-3-ylpyrazol-3-yl)cyclopropanecarboxamide |
| 240 | 1-(2-fluorophenyl)-N-(1-pyrimidin-2-ylpyrazol-3-yl)cyclopropanecarboxamide |
| 241 | 1-(2-fluorophenyl)-N-[1-(2-methylpyrimidin-5-yl)pyrazol-3-yl]cyclopropanecarboxamide |
| 242 | 1-(2-cyanophenyl)-N-(1-phenylpyrazol-3-yl)cyclopropanecarboxamide |
| 243 | 1-(3-cyanophenyl)-N-[1-(2-fluoro-4-pyridyl)pyrazol-3-yl]cyclopropanecarboxamide |
| 244 | 1-(3-cyanophenyl)-N-(1-phenylpyrazol-3-yl)cyclopropanecarboxamide |
| 245 | N-[1-(2-fluoro-4-pyridyl)pyrazol-3-yl]-1-(m-tolyl)cyclopropanecarboxamide |
| 246 | 1-(4-ethoxyphenyl)-N-[1-(2-fluoro-4-pyridyl)pyrazol-3-yl]cyclopropanecarboxamide |
| 247 | 1-(2-cyanophenyl)-N-[1-(2-fluoro-4-pyridyl)pyrazol-3-yl]cyclopropanecarboxamide |
| 248 | 1-(3,5-dimethylphenyl)-N-[1-(2-fIuoro-4-pyridyl)pyrazol-3-yl]cyclopropanecarboxamide |
| 249 | N-[1-(2-fluoro-4-pyridyl)pyrazol-3-yl]-1-(4-methoxyphenyl)cyclopropanecarboxamide |
| 250 | N-[1-(2-fluoro-4-pyridyl)pyrazol-3-yl]-1-(3-methoxyphenyl)cyclopropanecarboxamide |
| 251 | 1-(2-chlorophenyl)-N-[1-(2-fluoro-4-pyridyl)pyrazol-3-yl]cyclopropanecarboxamide |
| 252 | N-[1-(2-fluoro-4-pyridyl)pyrazol-3-yl]-1-(o-tolyl)cyclopropanecarboxamide |
| 253 | 1-(2,4-dichlorophenyl)-N-[1-(2-fluoro-4-pyridyl)pyrazol-3-yl]cyclopropanecarboxamide |
| 254 | 1-(3-fluorophenyl)-N-[1-(2-fluoro-4-pyridyl)pyrazol-3-yl]cyclopropanecarboxamide |
| 255 | 1-(4-fluorophenyl)-N-[1-(2-fluoro-4-pyridyl)pyrazol-3-yl]cyclopropanecarboxamide |
| 256 | 1-(3-chlorophenyl)-N-[1-(2-fluoro-4-pyridyl)pyrazol-3-yl]cyclopropanecarboxamide |
| 257 | 1-(4-chlorophenyl)-N-[1-(2-fluoro-4-pyridyl)pyrazol-3-yl]cyclopropanecarboxamide |
| 258 | N-[1-(4,6-dideuterio-2-methyl-pyrimidin-5-yl)pyrazol-3-yl]-1-(2-fluorophenyl)cyclopropanecarboxamide |
| 259 | 1-(2-fluorophenyl)-N-[1-(2-methoxy-4-pyridyl)pyrazol-3-yl]cyclopropanecarboxamide |
| 260 | 1-(2,5-difluorophenyl)-N-[1-(2-fluoro-4-pyridyl)pyrazol-3-yl]cyclopropanecarboxamide |
| 261 | 1-(2,5-difluorophenyl)-N-(1-phenylpyrazol-3-yl)cyclopropanecarboxamide |
| 262 | 1-(4-chloro-2-fluoro-phenyl)-N-[1-(2-fluoro-4-pyridyl)pyrazol-3-yl]cyclopropanecarboxamide |
| 263 | 1-(4-chloro-2-fluoro-phenyl)-N-(1-phenylpyrazol-3-yl)cyclopropanecarboxamide |
| 264 | 1-(5-chloro-2-fluoro-phenyl)-N-[1-(2-fluoro-4-pyridyl)pyrazol-3-yl]cyclopropanecarboxamide |
| 265 | 1-(5-chloro-2-fluoro-phenyl)-N-(1-phenylpyrazol-3-yl)cyclopropanecarboxamide |
| 266 | 1-(2,6-difluorophenyl)-N-[1-(2-fluoro-4-pyridyl)pyrazol-3-yl]cyclopropanecarboxamide |
| 267 | 1-(2,6-difluorophenyl)-N-(1-phenylpyrazol-3-yl)cyclopropanecarboxamide |
| 268 | 1-(2,3-difluorophenyl)-N-[1-(2-fluoro-4-pyridyl)pyrazol-3-yl]cyclopropanecarboxamide |
| 269 | 1-(2,3-difluorophenyl)-N-(1-phenylpyrazol-3-yl)cyclopropanecarboxamide |
| 270 | 1-(3,5-difluorophenyl)-N-[1-(2-fluoro-4-pyridyl)pyrazol-3-yl]cyclopropanecarboxamide |
| 271 | 1-(3,5-difluorophenyl)-N-(1-phenylpyrazol-3-yl)cyclopropanecarboxamide |
| 272 | 1-(2-chloro-6-fluoro-3-methyl-phenyl)-N-[1-(2-fluoro-4-pyridyl)pyrazol-3-yl] |
| | cyclopropanecarboxamide |
| 273 | 1-(2-chloro-6-fluoro-3-methyl-phenyl)-N-(1-phenylpyrazol-3-yl)cyclopropanecarboxamide |
| 274 | 1-(2-chloro-6-fluoro-phenyl)-N-[1-(2-fluoro-4-pyridyl)pyrazol-3-yl]cyclopropanecarboxamide |
| 275 | 1-(2-chloro-6-fluoro-phenyl)-N-(1-phenylpyrazol-3-yl)cyclopropanecarboxamide |
| 276 | 2,2,3,3-tetradeuterio-1-(2-fluorophenyl)-N-[1-(2-fluoro-4-pyridyl)pyrazol-3-yl] |
| | cyclopropanecarboxamide |
| 277 | 1-(3-f1uoro-2-pyridyl)-N-[1-(2-fluoro-4-pyridyl)pyrazol-3-yl]cyclopropanecarboxamide |
| 278 | 1-(3-fluoro-4-pyridyl)-N-(1-pyrimidin-4-ylpyrazol-3-yl)cyclopropanecarboxamide |
| 279 | 1-(3-fluoro-2-pyridyl)-N-(1-pyrimidin-4-ylpyrazol-3-yl)cyclopropanecarboxamide |
| 280 | 1-(3-fluoro-4-pyridyl)-N-(1-phenylpyrazol-3-yl)cyclopropanecarboxamide |
| 281 | 1-(3-fluoro-2-pyridyl)-N-(1-phenylpyrazol-3-yl)cyclopropanecarboxamide |
| 282 | N-[1-(2-fluoro-4-pyridyl)pyrazol-3-yl]-2-(5-methylpyrazol-1-yl)acetamide |
| 283 | N-[1-(2-fluoro-4-pyridyl)pyrazol-3-yl]-2-pyrazol-1-yl-acetamide |
| 284 | N-[1-(2-fluoro-4-pyridyl)pyrazol-3-yl]-2-(3-methylpyrazol-1-yl)acetamide |
| 285 | 1-(2,2-difluoro-1,3-benzodioxol-5-yl)-N-[1-(2-fluoro-4-pyridyl)pyrazol-3-yl] |
| | cyclopropanecarboxamide |
| 286 | N-[1-(2-fluoro-4-pyridyl)pyrazol-3-yl]-2-imidazol-1-yl-acetamide |
| 287 | N-[1-(2-fluoro-4-pyridyl)pyrazol-3-yl]-2-(4-methylpyrazol-1-yl)acetamide |
| 288 | 2-(5-methylpyrazol-1-yl)-N-(1-phenylpyrazol-3-yl)acetamide |
| 289 | N-(1-phenylpyrazol-3-yl)-2-pyrazol-1-yl-acetamide |
| 290 | 2-(4-methylpyrazol-1-yl)-N-(1-phenylpyrazol-3-yl)acetamide |
| 291 | 2-(3-methylpyrazol-1-yl)-N-(1-phenylpyrazol-3-yl)acetamide |
| 292 | 1-(2,2-difluoro-1,3-benzodioxo1-5-yl)-N-(1-phenylpyrazol-3-yl)cyclopropanecarboxamide |
| 293 | N-[1-(2-fluoro-4-pyridyl)pyrazol-3-yl]-2-(3-thienyl)acetamide |
| 294 | N-[1-(2-fluoro-4-pyridyl)pyrazol-3-yl]-2-methyl-2-phenyl-butanamide |
| 295 | N-[1-(2-fluoro-4-pyridyl)pyrazol-3-yl]-2-pyrrol-1-yl-acetamide |
| 296 | 2-(2-fluorophenyl)-2-methyl-N-(1-phenylpyrazol-3-yl)propanamide |
| 297 | N-(1-phenylpyrazol-3-yl)-2-pyrrol-1-yl-acetamide |
| 298 | N-(1-phenylpyrazol-3-yl)-2-(triazol-2-yl)acetamide |
| 299 | N-[1-(2-fluoro-4-pyridyl)pyrazol-3-yl]-2-(triazol-2-yl)acetamide |
| 300 | 2-methyl-2-phenyl-N-(1-phenylpyrazol-3-yl)butanamide |
| 301 | N-[1-(3,5-difluorophenyl)pyrazol-3-yl]-1-(2-fluorophenyl)cyclopropanecarboxamide |
| 302 | N-[1-(2-fluoro-4-pyridyl)pyrazol-3-yl]-2-(1-methylimidazol-2-yl)acetamide |
| 303 | N-[1-(2-fluoro-4-pyridyl)pyrazol-3-yl]-2-(1,2,4-triazol-1-yl)acetamide |
| 304 | N-[1-(2-fluoro-4-pyridyl)pyrazol-3-yl]-2-isoxazol-3-yl-acetamide |
| 305 | N-(1-phenylpyrazol-3-yl)-2-(1,2,4-triazol-1-yl)acetamide |
| 306 | 2-isoxazol-3-yl-N-(1-phenylpyrazol-3-yl)acetamide |
| 307 | N-[1-(2-fluoro-4-pyridyl)pyrazol-3-yl]-2-thiazol-2-yl-acetamide |
| 308 | N-[1-(3,5-difluorophenyl)pyrazol-3-yl]-1-phenyl-cyclopropanecarboxamide |
| 309 | N-[1-(2-fluoro-4-pyridyl)pyrazol-3-yl]-2-(1-methylpyrrol-2-yl)acetamide |
| 310 | N-(1-phenylpyrazol-3-yl)-2-thiazol-2-yl-acetamide |
| 311 | 2-(1-methylimidazol-2-yl)-N-(1-phenylpyrazol-3-yl)acetamide |
| 312 | 2-(1-methylpyrrol-2-yl)-N-(1-phenylpyrazol-3-yl)acetamide |
| 313 | 1-(3-fluoro-4-pyridyl)-N-[1-(2-fluoro-4-pyridyl)pyrazol-3-yl]cyclopropanecarboxamide |
| 314 | 1-(2-fluoro-5-methoxy-phenyl)-N-[1-(2-fluoro-4-pyridyl)pyrazol-3-yl]cyclopropanecarboxamide |
| 315 | 1-(2-fluorophenyl)-N-[1-[6-(trifluoromethyl)-3-pyridyl]pyrazol-3-yl]cyclopropanecarboxamide |
| 316 | 1-(2-fluorophenyl)-N-[1-[5-(trifluoromethyl)-3-pyridyl]pyrazol-3-yl]cyclopropanecarboxamide |
| 317 | 1-(2-fluorophenyl)-N-[1-(2-methoxy-3-pyridyl)pyrazol-3-yl]cyclopropanecarboxamide |
| 318 | N-[1-(5-cyano-3-pyridyl)pyrazol-3-yl]-1-(2-fluorophenyl)cyclopropanecarboxamide |
| 319 | 1-(2-fluorophenyl)-N-[1-(6-methoxy-3-pyridyl)pyrazol-3-yl]cyclopropanecarboxamide |
| 320 | N-[1-(2-cyano-3-pyridyl)pyrazol-3-yl]-1-(2-fluorophenyl)cyclopropanecarboxamide |
| 321 | 1-(2-fluorophenyl)-N-[1-(3,4,5-trimethoxyphenyl)pyrazol-3-yl]cyclopropanecarboxamide |
| 322 | N-[1-(3-fluoro-4-methoxy-phenyl)pyrazol-3-yl]-1-(2-fluorophenyl)cyclopropanecarboxamide |
| 323 | 1-(2-fluorophenyl)-N-[1-(5-methyl-3-pyridyl)pyrazol-3-yl]cyclopropanecarboxamide |
| 324 | 1-(2-fluorophenyl)-N-[1-[2-(trifluoromethyl)-4-pyridyl]pyrazol-3-yl]cyclopropanecarboxamide |
| 325 | N-[1-(5-chloro-3-pyridyl)pyrazol-3-yl]-1-(2-fluorophenyl)cyclopropanecarboxamide |
| 326 | N-[1-(6-cyano-3-pyridyl)pyrazol-3-yl]-1-(2-fluorophenyl)cyclopropanecarboxamide |
| 327 | N-[1-(3-cyano-5-methoxy-4-pyridyl)pyrazol-3-yl]-1-(2-fluorophenyl)cyclopropanecarboxamide |
| 328 | 1-(2-fluorophenyl)-N-[1-[3-(trifluoromethyl)-4-pyridyl]pyrazol-3-yl]cyclopropanecarboxamide |
| 329 | N-[1-(2-cyano-4-pyridyl)pyrazol-3-yl]-1-(2-fluorophenyl)cyclopropanecarboxamide |
| 330 | 1-(2-fluorophenyl)-N-[1-(2-methoxypyrimidin-4-yl)pyrazol-3-yl]cyclopropanecarboxamide |
| 331 | 1-(2-fluorophenyl)-N-[1-(2-methylpyrimidin-4-yl)pyrazol-3-yl]cyclopropanecarboxamide |
| 332 | 1-(2-fluorophenyl)-N-[1-[2-(2,2,2-trifluoro-1-hydroxy-ethyl)-4-pyridyl]pyrazol-3-yl] |
| | cyclopropanecarboxamide |
| 333 | N-[1-(3-chloro-4-pyridyl)pyrazol-3-yl]-1-(2-fluorophenyl)cyclopropanecarboxamide |
| 334 | 1-(2-fluorophenyl)-N-[1-[2-(1-hydroxy-1-methyl-ethyl)-4-pyridyl]pyrazol-3-yl] |
| | cyclopropanecarboxamide |
| 335 | N-[1-(3-cyano-4-pyridyl)pyrazol-3-yl]-1-(2-fluorophenyl)cyclopropanecarboxamide |
| 336 | N-[1-(3-cyano-2-methoxy-4-pyridyl)pyrazol-3-yl]-1-(2-fluorophenyl)cyclopropanecarboxamide |
| 337 | N-[1-(2,6-dimethylpyrimidin-4-yl)pyrazol-3-yl]-1-(2-fluorophenyl)cyclopropanecarboxamide |
| 338 | 1-(2-fluorophenyl)-N-[1-(2-methoxylpyrimidin-5-yl)pyrazol-3-yl]cyclopropanecarboxamide |
| 339 | N-[1-(4,6-dimethylpyrimidin-5-yl)pyrazol-3-yl]-1-(2-fluorophenyl)cyclopropanecarboxamide |
| 340 | 1-(2-fluorophenyl)-N-[1-(2-methoxy-3-methyl-4-pyridyl)pyrazol-3-yl]cyclopropanecarboxamide |
| 341 | 1-(2-fluorophenyl)-N-[1-(4-methylpyrimidin-5-yl)pyrazol-3-yl]cyclopropanecarboxamide |
| 342 | 1-(2-fluorophenyl)-N-[1-(2-isopropoxy-4-pyridyl)pyrazol-3-yl]cyclopropanecarboxamide |
| 343 | N-[1-(2,3-dimethyl-4-pyridyl)pyrazol-3-yl]-1-(2-fluorophenyl)cyclopropanecarboxamide |
| 344 | N-[1-(5-cyano-2-methyl-pyrimidin-4-yl)pyrazol-3-yl]-1-(2-fluorophenyl)cyclopropanecarboxamide |
| 345 | N-[1-(3-fluoro-5-methoxy-4-pyridyl)pyrazol-3-yl]-1-(2-fluorophenyl)cyclopropanecarboxamide |
| 346 | N-[1-(2-cyano-6-methyl-4-pyridyl)pyrazol-3-yl]-1-(2-fluorophenyl)cyclopropanecarboxamide |
| 347 | N-[1-(3,5-dimethoxy-4-pyridyl)pyrazol-3-yl]-1-(2-fluorophenyl)cyclopropanecarboxamide |
| 348 | N-[1-(5-fluoro-6-methoxy-3-pyridyl)pyrazol-3-yl]-1-(2-fluorophenyl)cyclopropanecarboxamide |
| 349 | N-[1-(5-fluoro-6-methyl-3-pyridyl)pyrazol-3-yl]-1-(2-fluorophenyl)cyclopropanecarboxamide |
| 350 | N-[1-(2,5-dimethyl-4-pyridyl)pyrazol-3-yl]-1-(2-fluorophenyl)cyclopropanecarboxamide |
| 351 | N-[1-(2-fluoro-3-methyl-4-pyridyl)pyrazol-3-yl]-1-(2-fluorophenyl)cyclopropanecarboxamide |
| 352 | N-[1-(3-cyano-5-fluoro-phenyl)pyrazol-3-yl]-1-(2-fluorophenyl)cyclopropanecarboxamide |
| 353 | 1-(2-fluorophenyl)-N-[1-(4-methoxypyrimidin-5-yl)pyrazol-3-yl]cyclopropanecarboxamide |
| 354 | N-[1-[5-fluoro-6-(1-hydroxy-1-methyl-ethyl)-3-pyridyl]pyrazol-3-yl]-1-(2-fluorophenyl) |
| | cyclopropanecarboxamide |
| 355 | N-[1-(3-fluoro-4-hydroxy-phenyl)pyrazol-3-yl]-1-(2-fluorophenyl)cyclopropanecarboxamide |
| 356 | 1-(2-fluorophenyl)-N-[1-(6-hydroxypyridazin-4-yl)pyrazol-3-yl]cyclopropanecarboxamide |
| 357 | N-[1-(3,5-difluoro-4-hydroxy-phenyl)pyrazol-3-yl]-1-(2-fluorophenyl)cyclopropanecarboxamide |
| 358 | 1-(2-fluorophenyl)-N-[1-(6-methoxypyrimidin-4-yl)pyrazol-3-yl]cyclopropanecarboxamide |
| 359 | 1-(2-fluorophenyl)-N-[1-(6-methoxy-2-methyl-pyrimidin-4-yl)pyrazol-3-yl]cyclopropanecarboxamide |
| 360 | 1-(2-fluorophenyl)-N-[1-(5-methylpyrimidin-4-yl)pyrazol-3-yl]cyclopropanecarboxamide |
| 361 | 1-(2-fluorophenyl)-N-[1-(5-fluoropyrimidin-4-yl)pyrazol-3-yl]cyclopropanecarboxamide |
| 362 | 2-(2-fluorophenyl)-N-methyl-N-(1-phenylpyrazol-3-yl)acetamide |
| 363 | 1-(2-fluorophenyl)-N-[1-(2-fluoro-4-pyridyl)pyrazol-3-yl]-N-methyl-cyclopropanecarboxamide |
| 364 | 1-(2-fluorophenyl)-N-[1-(2-hydroxy-4-pyridyl)pyrazol-3-yl]cyclopropanecarboxamide |
| 365 | N-[4-fluoro-1-(2-fluoro-4-pyridyl)pyrazol-3-yl]-1-(2-fluorophenyl)cyclopropanecarboxamide |
| 366 | Rel-(R)-2,2-difluoro-1-phenyl-N-(1-phenyl-1H-pyrazol-3-yl)cyclopropane-1-carboxamide |
| 367 | Rel-(S)-2,2-difluoro-1-phenyl-N-(1-phenyl-1H-pyrazol-3-yl)cyclopropane-1-carboxamide |
| 368 | Rel-(R)-2,2-difluoro-N-(1-(2-fluoropyridin-4-yl)-1H-pyrazol-3-yl)-1-phenylcyclopropane-1-carboxamide |
| 369 | Rel-(S)-2,2-difluoro-N-(1-(2-fluoropyridin-4-yl)-1H-pyrazol-3-yl)-1-phenylcyclopropane-1-carboxamide |
| 370 | Rel-(R)-2,2-dichloro-N-(1-(2-fluoropyridin-4-yl)-1H-pyrazol-3-yl)-1-phenylcyclopropane-1-carboxamide |
| 371 | Rel-(S)-2,2-dichloro-N-(1-(2-fluoropyridin-4-yl)-1H-pyrazol-3-yl)-1-phenylcyclopropane-1-carboxamide |
| 372 | 1-(3-fluorophenyl)-N-(4-methyl-1-phenyl-pyrazol-3-yl)cyclobutanecarboxamide |
| 373 | 1-(o-tolyl)-N-[1-(4-pyridyl)pyrazol-3-yl]cyclopropanecarboxamide |
| 374 | 1-(2-fluorophenyl)-N-[1-(4-methylthiazol-2-yl)pyrazol-3-yl]cyclopropanecarboxamide |
| 375 | 1-(2-fluorophenyl)-N-(1-oxazol-2-ylpyrazol-3-yl)cyclopropanecarboxamide |
| 376 | N-[1-(6-fluoro-5-methyl-3-pyridyl)pyrazol-3-yl]-1-(2-fluorophenyl)cyclopropanecarboxamide |
| 377 | 1-(2-fluorophenyl)-N-[1-(6-fluoro-3-pyridyl)pyrazol-3-yl]cyclopropanecarboxamide |
| 378 | N-[1-(5-chloro-6-methoxy-3-pyridyl)pyrazol-3-yl]-1-(2-fluorophenyl)cyclopropanecarboxamide |
| 379 | N-[1-(5,6-dichloro-3-pyridyl)pyrazol-3-yl]-1-(2-fluorophenyl)cyclopropanecarboxamide |
| 380 | N-[1-(5-bromopyrimidin-2-yl)pyrazol-3-yl]-1-(2-fluorophenyl)cyclopropanecarboxamide |
| 381 | 1-(2-fluorophenyl)-N-[1-[2-(trifluoromethyl)pyrimidin-5-yl]pyrazol-3-yl]cyclopropanecarboxamide |
| 382 | 1-(2-fluorophenyl)-N-[1-[2-(1-hydroxy-1-methyl-ethyl)pyrimidin-5-yl]pyrazol-3-yl]cyclopropanecarboxamide |
| 383 | N-[1-(2-tert-butylpyrimidin-5-yl)pyrazol-3-yl]-1-(2-fluorophenyl)cyclopropanecarboxamide |
| 384 | N-[1-(4-amino-3,5-difluoro-phenyl)pyrazol-3-yl]-1-(2-fluorophenyl)cyclopropanecarboxamide |
| 385 | N-[1-(3-amino-4-fluoro-phenyl)pyrazol-3-yl]-1-(2-fluorophenyl)cyclopropanecarboxamide |
| 386 | 1-(2-fluorophenyl)-N-(5-methyl-1-phenyl-pyrazol-3-yl)cyclopropanecarboxamide |
| 387 | N-[1-(4-amino-3-fluoro-phenyl)pyrazol-3-yl]-1-(2-fluorophenyl)cyclopropanecarboxamide |
| 388 | 1-(2-fluorophenyl)-N-[1-(thiadiazol-5-yl)pyrazol-3-yl]cyclopropanecarboxamide |
| 389 | N-[1-(2-fluoro-4-pyridyl)pyrazol-3-yl]-1-(2-methoxyphenyl)cyclopropanecarboxamide |
| 390 | 1-(2-fluorophenyl)-N-[1-(1-methylpyrazol-4-yl)pyrazol-3-yl]cyclopropanecarboxamide |
| 391 | N-[1-(3,5-difluoro-2-pyridyl)pyrazol-3-yl]-1-(2-fluorophenyl)cyclopropanecarboxamide |
| 392 | 1-(2-fluorophenyl)-N-[1-(1-methylimidazol-4-yl)pyrazol-3-yl]cyclopropanecarboxamide |
| 393 | N-[1-(2-amino-4-pyridyl)pyrazol-3-yl]-1-(2-fluorophenyl)cyclopropanecarboxamide |
| 394 | N-[1-[6-(dimethylamino)-3-pyridyl]pyrazol-3-yl]-1-(2-fluorophenyl)cyclopropanecarboxamide |
| 395 | N-[1-[2-(difluoromethoxy)-4-pyridyl]pyrazol-3-yl]-1-(2-fluorophenyl)cyclopropanecarboxamide |
| 396 | N-[1-[2-(difluoromethyl)-4-pyridyl]pyrazol-3-yl]-1-(2-fluorophenyl)cyclopropanecarboxamide |
| 397 | N-[1-[6-(difluoromethyl)-3-pyridyl]pyrazol-3-yl]-1-(2-fluorophenyl)cyclopropanecarboxamide |
| 398 | N-[1-(5-chloro-2-pyridyl)pyrazol-3-yl]-1-(2-fluorophenyl)cyclopropanecarboxamide |
| 399 | N-[1-(6-amino-5-fluoro-3-pyridyl)pyrazol-3-yl]-1-(2-fluorophenyl)cyclopropanecarboxamide |
| 400 | 1-(2-fluorophenyl)-N-[1-(1-methyltriazol-4-yl)pyrazol-3-yl]cyclopropanecarboxamide |
| 401 | 1-(2-fluorophenyl)-N-[1-(3-methylisothiazol-5-yl)pyrazol-3-yl]cyclopropanecarboxamide |
| 402 | 1-(2-fluorophenyl)-N-(1-isothiazol-3-ylpyrazol-3-yl)cyclopropanecarboxamide |
| 403 | N-[1-(1,3-dimethylpyrazol-4-yl)pyrazol-3-yl]-1-(2-fluorophenyl)cyclopropanecarboxamide |
| 404 | 1-(2-fluorophenyl)-N-[1-(5-methyl-1,3,4-thiadiazol-2-yl)pyrazol-3-yl]cyclopropanecarboxamide |
| 405 | N-[1-(1-ethylpyrazol-4-yl)pyrazol-3-yl]-1-(2-fluorophenyl)cyclopropanecarboxamide |
| 406 | 1-(2-fluorophenyl)-N-[1-(1,2,4-thiadiazol-5-yl)pyrazol-3-yl]cyclopropanecarboxamide |
| 407 | N-[1-(2-methoxy-4-pyridyl)pyrazol-3-yl]-1-pyrimidin-2-yl-cyclopropanecarboxamide |
| 408 | 1-(2-fluorophenyl)-N-[1-(2-methoxythiazol-5-yl)pyrazol-3-yl]cyclopropanecarboxamide |
| 409 | N-[1-[6-(difluoromethoxy)-3-pyridyl]pyrazol-3-yl]-1-(2-fluorophenyl)cyclopropanecarboxamide |
| 410 | N-[1-(2-chlorothiazol-5-yl)pyrazol-3-yl]-1-(2-fluorophenyl)cyclopropanecarboxamide |
| 411 | 1-(2-fluorophenyl)-N-[1-(3-methoxy-4-pyridyl)pyrazol-3-yl]cyclopropanecarboxamide |
| 412 | 1-(2-fluorophenyl)-N-[1-[6-(methylamino)-3-pyridyl]pyrazol-3-yl]cyclopropanecarboxamide |
| 413 | N-[1-(2,4-dimethylthiazol-5-yl)pyrazol-3-yl]-1-(2-fluorophenyl)cyclopropanecarboxamide |
| 414 | N-[1-[1-(difluoromethyl)-3-methyl-pyrazol-4-yl]pyrazol-3-yl]-1-(2-fluorophenyl)cyclopropanecarboxamide |
| 415 | 1-(2-fluorophenyl)-N-[1-(2-methyl-1,2,4-triazol-3-yl)pyrazol-3-yl]cyclopropanecarboxamide |
| 416 | 1-(2-fluorophenyl)-N-(1-isoxazol-3-ylpyrazol-3-yl)cyclopropanecarboxamide |
| 417 | 1-(2-fluorophenyl)-N-[1-(2-methylpyrazol-3-yl)pyrazol-3-yl]cyclopropanecarboxamide |
| 418 | N-[1-(3-chlorophenyl)pyrazol-3-yl]-1-(3-fluoro-2-pyridyl)cyclopropanecarboxamide |
| 419 | 2,2-difluoro-1-phenyl-N-[1-(4-pyridyl)pyrazol-3-yl]cyclopropanecarboxamide |
| 420 | 2,2-difluoro-N-[1-(5-fluoro-3-pyridyl)pyrazol-3-yl]-1-phenyl-cyclopropanecarboxamide |
| 421 | N-[1-(3-chlorophenyl)pyrazol-3-yl]-2,2-difluoro-1-phenyl-cyclopropanecarboxamide |
| 422 | 1-(3-fluoro-2-pyridyl)-N-[1-(4-pyridyl)pyrazol-3-yl]cyclopropanecarboxamide |
| 423 | 1-(3-fluoro-2-pyridyl)-N-[1-(5-fluoro-3-pyridyl)pyrazol-3-yl]cyclopropanecarboxamide |
| 424 | 1-(2-fluorophenyl)-N-(1-isoxazol-4-ylpyrazol-3-yl)cyclopropanecarboxamide |
| 425 | 1-(2-fluorophenyl)-N-[1-(1-methyl-1,2,4-triazol-3-yl)pyrazol-3-yl]cyclopropanecarboxamide |
| 426 | 1-(2-fluorophenyl)-N-[1-(3-methylimidazol-4-yl)pyrazol-3-yl]cyclopropanecarboxamide |
| 427 | 1-(2-fluorophenyl)-N-[1-(4-methyl-1,2,4-triazol-3-yl)pyrazol-3-yl]cyclopropanecarboxamide |
| 428 | N-[1-(2,4-dimethoxyphenyl)pyrazol-3-yl]-1-(2-fluorophenyl)cyclopropanecarboxamide |
| 429 | N-[1-(4-fluoro-2-methoxy-phenyl)pyrazol-3-yl]-1-(2-fluorophenyl)cyclopropanecarboxamide |
| 430 | N-[1-(6-ethoxy-3-pyridyl)pyrazol-3-yl]-1-(2-fluorophenyl)cyclopropanecarboxamide |
| 431 | N-[1-[5-fluoro-6-(methylamino)-3-pyridyl]pyrazol-3-yl]-1-(2-fluorophenyl)cyclopropanecarboxamide |
| 432 | 1-(2-fluorophenyl)-N-[1-[6-(trideuteriomethoxy)pyridazin-4-yl]pyrazol-3-yl]cyclopropanecarboxamide |
| 433 | Rel-(S)-2,2-difluoro-1-phenyl-N-[1-(4-pyridyl)pyrazol-3-yl]cyclopropanecarboxamide |
| 434 | Rel-(R)-2,2-difluoro-1-phenyl-N-[1-(4-pyridyl)pyrazol-3-yl]cyclopropanecarboxamide |
| 435 | Rel-(S)-2,2-difluoro-N-[1-(5-fluoro-3-pyridyl)pyrazol-3-yl]-1-phenyl-cyclopropanecarboxamide |
| 436 | Rel-(R)-2,2-difluoro-N-[1-(5-fluoro-3-pyridyl)pyrazol-3-yl]-1-phenyl-cyclopropanecarboxamide |
| 437 | Rel-(S)-N-[1-(3-chlorophenyl)pyrazol-3-yl]-2,2-difluoro-1-phenyl-cyclopropanecarboxamide |
| 438 | Rel-(R)-N-[1-(3-chlorophenyl)pyrazol-3-yl]-2,2-difluoro-1-phenyl-cyclopropanecarboxamide |
| 439 | 1-(2-fluorophenyl)-N-[1-[2-(trideuteriomethoxy)-4-pyridyl]pyrazol-3-yl]cyclopropanecarboxamide |
| 440 | 1-(2-fluorophenyl)-N-[1-[6-(trideuteriomethoxy)-3-pyridyl]pyrazol-3-yl]cyclopropanecarboxamide |
| 441 | 1-(2-fluorophenyl)-N-[1-[5-fluoro-6-(trideuteriomethoxy)-3-pyridyl]pyrazol-3-yl]cyclopropanecarboxamide |
| 442 | N-[5-fluoro-1-(5-fluoro-6-methoxy-3-pyridyl)pyrazol-3-yl]-1-(2-fluorophenyl)cyclopropanecarboxamide |
| 443 | 1-(2-fluorophenyl)-N-[1-(5-methyl-1,3,4-oxadiazol-2-yl)pyrazol-3-yl]cyclopropanecarboxamide |
| 444 | 2-(hydroxymethyl)-1-phenyl-N-(1-phenylpyrazol-3-yl)cyclopropanecarboxamide |
| 445 | N-[4-fluoro-1-(5-fluoro-3-pyridyl)pyrazol-3-yl]-1-phenyl-cyclopropanecarboxamide |
| 446 | N-[1-(6-chloropyridazin-4-yl)pyrazol-3-yl]-1-(2-fluorophenyl)cyclopropanecarboxamide |
| 447 | 1-(2-fluorophenyl)-N-[1-(6-methoxypyridazin-4-yl)pyrazol-3-yl]cyclopropanecarboxamide |
| 448 | N-[1-(2,4-difluorophenyl)pyrazol-3-yl]-1-(2-fluorophenyl)cyclopropanecarboxamide |
| 449 | N-[1-(2-ethoxy-4-pyridyl)pyrazol-3-yl]-1-(2-fluorophenyl)cyclopropanecarboxamide |
| 450 | N-[1-(4-fluoro-2-methyl-phenyl)pyrazol-3-yl]-1-(3-fluoro-2-pyridyl)cyclopropanecarboxamide |
| 451 | N-[1-(2,4-difluorophenyl)-5-methyl-pyrazol-3-yl]-1-(3-fluoro-2-pyridyl)cyclopropanecarboxamide |
| 452 | N-[1-(5-fluoro-6-methoxy-3-pyridyl)pyrazol-3-yl]-1-(3-fluoro-2-pyridyl)cyclopropanecarboxamide |
| 453 | N-[1-(3-chloro-2-pyridyl)pyrazol-3-yl+-1-(3-fluoro-2-pyridyl)cyclopropanecarboxamide |
| 454 | 1-(3-fluoro-2-pyridyl)-N-[1-(2-methoxypyrimidin-5-yl)pyrazol-3-yl]cyclopropanecarboxamide |
| 455 | N-[1-(5-chloro-3-pyridyl)pyrazol-3-yl]-1-(3-fluoro-2-pyridyl)cyclopropanecarboxamide |
| 456 | N-[1-(4-fluorophenyl)pyrazol-3-yl]-1-(3-fluoro-2-pyridyl)cyclopropanecarboxamide |
| 457 | 1-(3-fluoro-2-pyridyl)-N-[1-[4-(trifluoromethyl)phenyl]pyrazol-3-yl]cyclopropanecarboxamide |
| 458 | 1-(3-fluoro-2-pyridyl)-N-(1-pyridazin-3-ylpyrazol-3-yl)cyclopropanecarboxamide |
| 459 | 1-(3-fluoro-2-pyridyl)-N-(1-pyrimidin-5-ylpyrazol-3-yl)cyclopropanecarboxamide |
| 460 | 1-(3-fluoro-2-pyridyl)-N-[1-(3-fluoro-2-pyridyl)pyrazol-3-yl]cyclopropanecarboxamide |
| 461 | N-[1-(3,5-difluorophenyl)pyrazol-3-yl]-1-(3-fluoro-2-pyridyl)cyclopropanecarboxamide |
| 462 | N-[5-fluoro-1-(5-fluoro-3-pyridyl)pyrazol-3-yl]-1-(3-fluoro-2-pyridyl)cyclopropanecarboxamide |
| 463 | N-[5-fluoro-1-(5-fluoro-3-pyridyl)pyrazol-3-yl]-1-(2-fluorophenyl)cyclopropanecarboxamide |
| 464 | N-[1-(5-fluoro-3-pyridyl)pyrazol-3-yl]-1-(2-pyridyl)cyclopropanecarboxamide |
| 465 | N-[1-(5-fluoro-3-pyridyl)pyrazol-3-yl]-1-[3-trifluoromethoxy)phenyl]cyclopropanecarboxamide |
| 466 | 1-(5-fluoro-2-pyridyl)-N-[1-(5-fluoro-3-pyridyl)pyrazol-3-yl]cyclopropanecarboxamide |
| 467 | N-[1-(5-fluoro-2-methoxy-4-pyridyl)pyrazol-3-yl]-1-(2-fluorophenyl)cyclopropanecarboxamide |
| 468 | N-[1-(4-fluoro-3-methoxy-phenyl)pyrazol-3-yl]-1-(2-fluorophenyl)cyclopropanecarboxamide |
| 469 | 1-(2-fluorophenyl)-N-[1-(4-methoxy-3-pyridyl)pyrazol-3-yl]cyclopropanecarboxamide |
| 470 | N-[1-(2-fluoro-4-methoxy-phenyl)pyrazol-3-yl]-1-(2-fluorophenyl)cyclopropanecarboxamide |
| 471 | 1-(2-fluorophenyl)-N-[1-(2-methyl-3-pyridyl)pyrazol-3-yl]cyclopropanecarboxamide |
| 472 | 1-(2-fluorophenyl)-N-[1-(4-methyl-3-pyridyl)pyrazol-3-yl]cyclopropanecarboxamide |
| 473 | 2-(3-fluoro-2-pyridyl)-N-[1-(5-fluoro-3-pyridyl)pyrazol-3-yl]spiro[2.2]pentane-2-carboxamide |
| 474 | N-(1-phenylpyrazol-3-yl)-1-(2-thienyl)cyclopropanecarboxamide |
| 475 | 1-(5-fluoro-2-pyridyl)-N-(1-phenylpyrazol-3-yl)cyclopropanecarboxamide |
| 476 | 2-methyl-N-(1-phenylpyrazol-3-yl)-2-(2-pyridyl)propanamide |
| 477 | N-(1-phenylpyrazol-3-yl)-1-pyrazin-2-yl-cyclopropanecarboxamide |
| 478 | N-[1-(2-methoxy-4-pyridyl)pyrazol-3-yl]-1-(2-thienyl)cyclopropanecarboxamide |
| 479 | N-[1-(3,4-difluorophenyl)pyrazol-3-yl]-1-(2-thienyl)cyclopropanecarboxamide |
| 480 | N-[1-(3,5-difluorophenyl)pyrazol-3-yl]-1-(2-thienyl)cyclopropanecarboxamide |
| 481 | N-[1-(4-fluorophenyl)pyrazol-3-yl]-1-(2-thienyl)cyclopropanecarboxamide |
| 482 | N-[1-(4-pyridyl)pyrazol-3-yl]-1-(2-thienyl)cyclopropanecarboxamide |
| 483 | N-[1-(5-fluoro-3-pyridyl)pyrazol-3-yl]-1-(2-thienyl)cyclopropanecarboxamide |
| 484 | N-[1-[5-(difluoromethoxy)-3-pyridyl]pyrazol-3-yl]-1-(2-thienyl)cyclopropanecarboxamide |
| 485 | 1-(5-fluoro-2-pyridyl)-N-[1-(2-methoxy-4-pyridyl)pyrazol-3-yl]cyclopropanecarboxamide |
| 486 | N-[1-(3,4-difluorophenyl)pyrazol-3-yl]-1-(5-fluoro-2-pyridyl)cyclopropanecarboxamide |
| 487 | N-[1-(3,5-difluorophenyl)pyrazol-3-yl]-1-(5-fluoro-2-pyridyl)cyclopropanecarboxamide |
| 488 | N-[1-(4-fluorophenyl)pyrazol-3-yl]-1-(5-fluoro-2-pyridyl)cyclopropanecarboxamide |
| 489 | 1-(5-fluoro-2-pyridyl)-N-[1-(4-pyridyl)pyrazol-3-yl]cyclopropanecarboxamide |
| 490 | N-[1-[5-(difluoromethoxy)-3-pyridyl]pyrazol-3-yl]-1-(5-fluoro-2-pyridyl)cyclopropanecarboxamide |
| 491 | N-[1-(2-methoxy-4-pyridyl)pyrazol-3-yl]-1-pyrazin-2-yl-cyclopropanecarboxamide |
| 492 | N-[1-(3,4-difluorophenyl)pyrazol-3-yl]-1-pyrazin-2-yl-cyclopropanecarboxamide |
| 493 | N-[1-(3,5-difluorophenyl)pyrazol-3-yl]-1-pyrazin-2-yl-cyclopropanecarboxamide |
| 494 | N-[1-(4-fluorophenyl)pyrazol-3-yl]-1-pyrazin-2-yl-cyclopropanecarboxamide |
| 495 | 1-pyrazin-2-yl-N-[1-(4-pyridyl)pyrazol-3-yl]cyclopropanecarboxamide |
| 496 | N-[1-[5-(difluoromethoxy)-3-pyridyl]pyrazol-3-yl]-1-pyrazin-2-yl-cyclopropanecarboxamide |
| 497 | 1-(3-fluoro-5-methyl-2-pyridyl)-N-[1-(5-fluoro-3-pyridyl)pyrazol-3-yl]cyclopropanecarboxamide |
| 498 | 1-(5-chloro-3-fluoro-2-pyridyl)-N-[1-(5-fluoro-3-pyridyl)pyrazol-3-yl]cyclopropanecarboxamide |
| 499 | Rel-(S)-2-(3-fluoro-2-pyridyl)-N-[1-(5-fluoro-3-pyridyl)pyrazol-3-yl]spiro[2.2]pentane-2-carboxamide |
| 500 | Rel-(R)-2-(3-fluoro-2-pyridyl)-N-[1-(5-fluoro-3-pyridyl)pyrazol-3-yl]spiro[2.2]pentane-2-carboxamide |
| 501 | 1-(2-fluorophenyl)-N-[1-(4-methoxy-2-pyridyl)pyrazol-3-yl]cyclopropanecarboxamide |
| 502 | 1-(2-fluorophenyl)-N-[1-(6-methoxy-2-pyridyl)pyrazol-3-yl]cyclopropanecarboxamide |
| 503 | 1-(3-fluoro-2-pyridyI)-N-[1-(5-fluoro-3-pyridyl)pyrazol-3-yl]-2-methyl-cyclopropanecarboxamide |
| 504 | N-[1-(5-chloro-3-pyridyl)pyrazol-3-yl]-1-(3-fluoro-2-pyridyl)-2-methyl-cyclopropanecarboxamide |
| 505 | N-[1-(4-fluorophenyl)pyrazol-3-yl]-1-(3-fluoro-2-pyridyl)-2-methyl-cyclopropanecarboxamide |
| 506 | N-[1-(3,4-difluorophenyl)pyrazol-3-yl]-1-(3-fluoro-2-pyridyl)-2-methyl-cyclopropanecarboxamide |
| 507 | N-[1-(2,4-difluorophenyl)-5-methyl-pyrazol-3-yl]-1-(3-fluoro-2-pyridyl)- |
| | 2-methyl-cyclopropanecarboxamide |
| 508 | N-[1-(3,5-difluorophenyl)pyrazol-3-yl]-1-(3-fluoro-2-pyridyl)-2-methyl-cyclopropanecarboxamide |
| 509 | N-[1-(5-fluoro-6-methoxy-3-pyridyl)pyrazol-3-yl]-1-(3-fluoro-2-pyridyl)- |
| | 2-methyl-cyclopropanecarboxamide |
| 510 | 1-(3-fluoro-2-pyridyl)-N-[1-(2-methoxy-4-pyridyl)pyrazol-3-yl]-2-methyl-cyclopropanecarboxamide |
| 511 | 1-(3-fluoro-2-pyridyl)-N-[1-(5-fluoro-3-pyridyl)pyrazol-3-yl]-2,2-dimethyl-cyclopropanecarboxamide |
| 512 | N-[1-(5-chloro-3-pyridyl)pyrazol-3-yl]-1-(3-fluoro-2-pyridyl)-2,2-dimethyl-cyclopropanecarboxamide |
| 513 | N-[1-(4-fluorophenyl)pyrazol-3-yl]-1-(3-fluoro-2-pyridyl)-2,2-dimethyl-cyclopropanecarboxamide |
| 514 | N-[1-(3,4-difluorophenyl)pyrazol-3-yl]-1-(3-fluoro-2-pyridyl)-2,2-dimethyl-cyclopropanecarboxamide |
| 515 | N-[1-(2,4-difluorophenyl)-5-methyl-pyrazol-3-yl]-1-(3-fluoro-2-pyridyl)- |
| | 2,2-dimethyl-cyclopropanecarboxamide |
| 516 | N-[1-(3,5-difluorophenyl)pyrazol-3-yl]-1-(3-fluoro-2-pyridyl)-2,2-dimethyl-cyclopropanecarboxamide |
| 517 | N-[1-(5-fluoro-6-methoxy-3-pyridyl)pyrazol-3-yl]-1-(3-fluoro-2-pyridyl)- |
| | 2,2-dimethyl-cyclopropanecarboxamide |
| 518 | 1-(3-fluoro-2-pyridyl)-2,2-dimethyl-N-[1-(4-pyridyl)pyrazol-3-yl]cyclopropanecarboxamide |
| 519 | 1-(3-fluoro-2-pyridyl)-N-[1-(2-methoxy-4-pyridyl)pyrazol-3-yl]-2,2-dimethyl-cyclopropanecarboxamide |
| 520 | N-[1-(5-chloro-3-pyridyl)pyrazol-3-yl]-2-(3-fluoro-2-pyridyl)spiro[2.2]pentane-2-carboxamide |
| 521 | N-[1-(4-fluorophenyl)pyrazol-3-yl]-2-(3-fluoro-2-pyridyl)spiro[2.2]pentane-2-carboxamide |
| 522 | N-[1-(3,4-difluorophenyl)pyrazol-3-yl]-2-(3-fluoro-2-pyridyl)spiro[2.2]pentane-2-carboxamide |
| 523 | N-[1-(2,4-difluorophenyl)-5-methyl-pyrazol-3-yl]-2-(3-fluoro-2-pyridyl)spiro[2.2]pentane-2-carboxamide |
| 524 | N-[1-(3,5-difluorophenyl)pyrazol-3-yl]-2-(3-fluoro-2-pyridyl)spiro[2.2]pentane-2-carboxamide |
| 525 | N-[1-(5-fluoro-6-methoxy-3-pyridyl)pyrazol-3-yl]-2-(3-fluoro-2-pyridyl)spiro[2.2]pentane-2-carboxamide |
| 526 | 2-(3-fluoro-2-pyridyl)-N-[1-(4-pyridyl)pyrazol-3-yl]spiro[2.2]pentane-2-carboxamide |
| 527 | 2-(3-fluoro-2-pyridyl)-N-[1-(2-methoxy-4-pyridyl)pyrazol-3-yl]spiro[2.2]pentane-2-carboxamide |
| 528 | 1-(3-fluoro-2-pyridyl)-2-methyl-N-[1-(4-pyridyl)pyrazol-3-yl]cyclopropanecarboxamide |
| 529 | N-[1-(5-fluoro-3-pyridyl)pyrazol-3-yl]-1-pyrimidin-2-yl-cyclopropanecarboxamide |
| 530 | N-[1-(5-chloro-3-pyridyl)pyrazol-3-yl]-1-pyrimidin-2-yl-cyclopropanecarboxamide |
| 531 | N-[1-(4-fluorophenyl)pyrazol-3-yl]-1-pyrimidin-2-yl-cyclopropanecarboxamide |
| 532 | N-[1-(3,4-difluorophenyl)pyrazol-3-yl]-1-pyrimidin-2-yl-cyclopropanecarboxamide |
| 533 | N-[1-(2,4-difluorophenyl)-5-methyl-pyrazol-3-yl]-1-pyrimidin-2-yl-cyclopropanecarboxamide |
| 534 | N-[1-(3,5-difluorophenyl)pyrazol-3-yl]-1-pyrimidin-2-yl-cyclopropanecarboxamide |
| 535 | N-[1-(5-fluoro-6-methoxy-3-pyridyl)pyrazol-3-yl]-1-pyrimidin-2-yl-cyclopropanecarboxamide |
| 536 | N-[1-(4-pyridyl)pyrazol-3-yl]-1-pyrimidin-2-yl-cyclopropanecarboxamide |

*Throughout, the prefix “rel-” is used to indicate an enantiomer of unknown absolute configuration (see also IUPAC Commission on Nomenclature of Organic Chemistry, Recommendations 1974, Rule E-4.10, Pure & Appl. Chem., 45:11-30; and H.A. Favre and W. H. Powell, Nomenclature of Organic Chemistry: IUPAC Recommendations and Preferred Names 2013, 2013, Royal Society of Chemistry, pp. 1210-1212). For compounds containing only one stereocenter, the (R) or (S) designation is assigned arbitrarily.

As used herein, the term “including” and other forms thereof such as “include”, “includes”, etc. are intended to be open-ended unless otherwise specified or clear from context. That is, “including” is to be understood as “including but not limited to” unless otherwise specified or clear from context. The phrase “such as” is similarly intended to be open-ended unless otherwise specified or clear from context.

As used herein, the term “very long chain fatty acids” (VLCFA) refers to fatty acid moieties having greater than or equal to 22 carbons in the carbon chain length (e.g., at least 22, 23, 24, 25, 26, 27, 28, 29, or 30 carbons long) of the main fatty acid side chain and can be saturated (i.e., without double-bonds; also called straight-chain) or unsaturated (e.g., monounsaturated with 1 double bond or polyunsaturated with at least 2 double bonds).

In some embodiments, VLCFA refers to fatty acid moieties having greater than or equal to 24 carbons in the carbon chain length (e.g., at least 24, 25, 26, 27, 28, 29, or 30 carbons long) of the main fatty acid side chain and are saturated. In some embodiments, VLCFA refers to fatty acid moieties having 26 carbons in the carbon chain of the main fatty acid side chain and are saturated.

A non-limiting example of VLCFA is a straight-chain VLCFA such as lignocerotic acid, which is a C24:0 straight-chain VLCFA, and cerotic acid, which is a C26:0 straight-chain VLCFA. It is understood by one of ordinary skill in the art that C##:# means that there are ##-number of carbons in the carbon chain-length and that there is # instances of double-bonds in the carbon chain. Thus, C26:0 means that the carbon chain of the VLCFA has 26 carbons in the carbon chain-length and zero instances of double-bonds in the carbon chain. VCLFA include straight-chain VLCFA (SC-VLCFA) and VLCFA incorporation products (i.e., fatty-acid moieties that are generated from SC-VLCFA by incorporating SC-VLCFA into their structure), such as, but not limited to, lysophosphatidylcholines (LPC), sphingomyelins (SM), acyl carnitines, cholesterol esters, and ceramides. LPC VLCFA are generated from straight chain VLCFA (SC-VLCFA) and are used clinically for newborn screening (Vogel et al., Mol. Genet. Metab. (2015) 114(4):599-603). The chemical entities, compositions thereof, and methods of using any of the foregoing, as described further herein, are useful for reduction of VLCFA levels in the CSF, blood, skin oil, brain, adrenal gland, nerve, adipose, muscle, liver, and/or other tissues. In some embodiments, the methods described herein are useful for reduction of VLCFA levels wherein the VLCFA are unsaturated. In some embodiments, the methods described herein are useful for reduction of VLCFA levels wherein the VLCFA are saturated (also called straight-chain). In some embodiments, the methods described herein are useful for reduction of VLCFA levels wherein the VLCFA are monounsaturated. In some embodiments, the methods described herein are useful for reduction of VLCFA levels wherein the VLCFA are polyunsaturated. In some embodiments, the methods described herein are useful for reduction of VLCFA levels, wherein the VLFCA are SC-VLCFA. In some embodiments, the methods described herein are useful for reduction of VLCFA levels, wherein the VLFCA are VLCFA incorporation products. In some embodiments, the methods described herein are useful for reduction of VLCFA levels, wherein the VLFCA are LPC. In some embodiments, the methods described herein are useful for reduction of a VLCFA level, wherein the VLCFA has at least 24 carbons in the chain length, at least 26 carbons, at least 28 carbons, or at least 30 carbons in the chain length. In some embodiments, the methods described herein are useful for reduction of a VLCFA level, wherein the VLCFA has 26 carbons in the chain length. In some embodiments, the methods described herein are useful for reduction of VLCFA levels, wherein the VLFCA are C24:0 SC-VLCFA or C26:0 SC-VLCFA. In some embodiments, the methods described herein are useful for reduction of VLCFA levels, wherein the VLFCA are C24:0 LPC or C26:0 LPC. As used herein, the phrase “reduction of VLCFA levels” or “reduction of a VLCFA level” means reduction of at least one or more types of VLCFA (which include VLCFA incorporation products) and optionally can be further specified in context. In some embodiments, reduction of VLCFA levels means that the levels of VLCFA in the cell or patient, following treatment with one or more chemical entities described herein, are reduced compared to the baseline levels of VLCFA before treatment with the chemical entities described herein. In some embodiments, the reduction of VLCFA levels means that the levels of VLCFA for cells or patients, either directly or via a sample, are reduced by at least about 25%, or at least by about 30%, or at least by about 33%, or by about 30% to about 80% relative to the baseline untreated levels after the cell or patient are treated the chemical entities described herein.

As used here, phrases such as deficiency of a protein (e.g., ABCD1 protein, ACOX1, ACBD5, and DBP) means that there are mutations that lead, for example, to a loss of protein expression or to a loss of protein function, or to a loss of protein trafficking to its place of function, or to two or all of these losses.

Compounds of this invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, HANDBOOK OF CHEMISTRY AND PHYSICS, 75^{th} Ed. Additionally, general principles of organic chemistry are described in M. Loudon and J. Parise, ORGANIC CHEMISTRY, 6^{th} Ed., W.H. Freeman & Co.: New York (2016), and M. B. Smith, MARCH'S ADVANCED ORGANIC CHEMISTRY, 7^{th} Ed., John Wiley & Sons, Inc.: Hoboken (2013), the entire contents of each of which are hereby incorporated by reference.

As described herein, a specified number range of atoms includes any integer therein. For example, a group having from 1-4 atoms could have 1, 2, 3, or 4 atoms.

As described herein, compounds of the invention may optionally be substituted with one or more substituents, such as are illustrated generally herein, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

Unless otherwise indicated, a substituent connected by a bond drawn from the center of a ring means that the substituent can be bonded to any position in the ring. In example (i) below, for instance, J can be bonded to any position on the pyridyl ring. For bicyclic rings, a bond drawn through both rings indicates that the substituent can be bonded from any position of the bicyclic ring. In example (ii) below, for instance, J can be bonded to the 5-membered ring (on the nitrogen atom, for instance), and to the 6-membered ring.

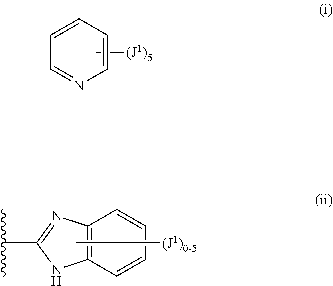

The term “stable”, as used herein, refers to compounds that are not substantially altered when subjected to conditions to allow for their production, detection, recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group”, as used herein, means a straight-chain (i.e., unbranched) or branched, substituted or unsubstituted, hydrocarbon chain that is completely saturated or that contains one or more units of unsaturation that has a single point of attachment to the rest of the molecule.

Unless otherwise specified, aliphatic groups contain 1-20 aliphatic carbon atoms. In some embodiments, aliphatic groups contain 1-10 aliphatic carbon atoms. In some embodiments, aliphatic groups contain 1-8 aliphatic carbon atoms. In some embodiments, aliphatic groups contain 1-6 aliphatic carbon atoms. In some embodiments, aliphatic groups contain 1-4 aliphatic carbon atoms. Aliphatic groups may be linear or branched, substituted or unsubstituted alkyl, alkenyl, or alkynyl groups. Specific examples include methyl, ethyl, isopropyl, n-propyl, sec-butyl, vinyl, n-butenyl, ethynyl, and tert-butyl.

The term “cycloaliphatic” (or “carbocycle” or “carbocyclyl”) refers to a monocyclic C₃-C₈ hydrocarbon or bicyclic C₈-C₁₂ hydrocarbon that is completely saturated or that contains one or more units of unsaturation, but which is not aromatic, that has a single point of attachment to the rest of the molecule wherein any individual ring in said bicyclic ring system has 3-7 members. Examples of cycloaliphatic groups include cycloalkyl and cycloalkenyl groups. Specific examples include cyclohexyl, cyclopropenyl, and cyclobutyl.

The term “heterocycle”, “heterocyclyl”, or “heterocyclic” as used herein means non-aromatic, monocyclic, bicyclic, or tricyclic ring systems in which one or more ring members are an independently selected heteroatom. In some embodiments, the “heterocycle”, “heterocyclyl”, or “heterocyclic” group has three to fourteen ring members in which one or more ring members is a heteroatom independently selected from oxygen, sulfur, nitrogen, or phosphorus, and each ring in the system contains 3 to 7 ring members.

Examples of heterocycles include 3-1H-benzimidazol-2-one, 3-(1-alkyl)-benzimidazol-2-one, 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolane, benzodithiane, and 1,3-dihydro-imidazol-2-one.

Cyclic groups, (e.g. cycloaliphatic and heterocycles), can be linearly fused, bridged, or spirocyclic.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation. Examples of unsaturated groups include propyne, butene, cyclohexene, tetrahydropyridine and cyclooctatetraene. The term “alkoxy”, or “thioalkyl”, as used herein, refers to an alkyl group, as previously defined, attached through an oxygen (“alkoxy”) or sulfur (“thioalkyl”) atom.

The terms “haloalkyl” (e.g., haloC₁₋₄alkyl), “haloalkenyl”, “haloaliphatic”, and “haloalkoxy” mean alkyl, alkenyl or alkoxy, as the case may be, substituted with one or more halogen atoms. This term includes perfluorinated alkyl groups, such as —CF₃ and —CF₂CF₃.

The terms “halogen”, “halo”, and “hal” mean F, Cl, Br, or I.

The term “aryl” used alone or as part of a larger moiety as in “aralkyl”, “aralkoxy”, or “aryloxyalkyl”, refers to carbocyclic aromatic ring systems. The term includes monocyclic, bicyclic, and tricyclic ring systems having a total of five to fourteen ring members, wherein at least one ring in the system is aromatic and wherein each ring in the system contains 3 to 7 ring members. The term “aryl” may be used interchangeably with the term “aryl ring”.

The term “heteroaryl”, used alone or as part of a larger moiety as in “heteroaralkyl” or “heteroarylalkoxy”, refers to monocyclic, bicyclic, and tricyclic ring systems having a total of five to fourteen ring members, wherein at least one ring in the system is aromatic, at least one ring in the system contains one or more heteroatoms, and wherein each ring in the system contains 3 to 7 ring members. The term “heteroaryl” may be used interchangeably with the term “heteroaryl ring” or the term “heteroaromatic”. Examples of heteroaryl rings include 2-furanyl, 3-furanyl, N-imidazolyl, 2-imidazolyl, 4-imidazolyl, 5-imidazolyl, benzimidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

It should be understood that the term “heteroaryl” includes certain types of heteroaryl rings that exist in equilibrium between two different forms. More specifically, for example, species such as hydropyridine and pyridinone (and likewise hydroxypyrimidine and pyrimidinone) are meant to be encompassed within the definition of “heteroaryl.”

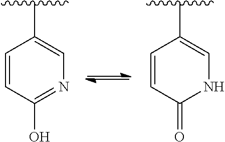 [SMILES: *c1ccc(O)nc1 ; *c1ccc(=O)[nH]c1]

The terms “protecting group” and “protective group” as used herein, are interchangeable and refer to an agent used to temporarily block one or more desired functional groups in a compound with multiple reactive sites. In certain embodiments, a protecting group has one or more, or preferably all, of the following characteristics: a) is added selectively to a functional group in good yield to give a protected substrate that is b) stable to reactions occurring at one or more of the other reactive sites; and c) is selectively removable in good yield by reagents that do not attack the regenerated, deprotected functional group. As would be understood by one skilled in the art, in some cases, the reagents do not attack other reactive groups in the compound. In other cases, the reagents may also react with other reactive groups in the compound. Examples of protecting groups are detailed in Greene, T. W., Wuts, P. G in “Protective Groups in Organic Synthesis”, Third Edition, John Wiley & Sons, New York: 1999 (“Greene”) (and other editions of the book), the entire contents of which are hereby incorporated by reference. The term “nitrogen protecting group”, as used herein, refers to an agent used to temporarily block one or more desired nitrogen reactive sites in a multifunctional compound. Preferred nitrogen protecting groups also possess the characteristics exemplified for a protecting group above, and certain exemplary nitrogen protecting groups are also detailed in Chapter 7 in Greene.

In some embodiments, a methylene or carbon unit of an alkyl or aliphatic chain is optionally replaced with another atom or group. Examples of such atoms or groups include nitrogen, oxygen, sulfur, —C(O)—, —C(═N—CN)—, —C(═NR)—, —C(═NOR)—, —SO—, and —SO₂—. These atoms or groups can be combined to form larger groups. Examples of such larger groups include —OC(O)—, —C(O)CO—, —CO₂—, —C(O)NR—, —C(═N—CN), —NRCO—, —NRC(O)O—, —SO₂NR—, —NRSO₂—, —NRC(O)NR—, —OC(O)NR—, and —NRSO₂NR—, wherein R is, for example, H or C₁₋₆ aliphatic. It should be understood that these groups can be bonded to the methylene or carbon units of the aliphatic chain via single, double, or triple bonds. An example of an optional replacement (nitrogen atom in this case) that is bonded to the aliphatic chain via a double bond would be —CH₂CH═N—CH₃. In some cases, especially on the terminal end, an optional replacement can be bonded to the aliphatic group via a triple bond. One example of this would be CH₂CH₂CH₂C≡N. It should be understood that in this situation, the terminal nitrogen is not bonded to another atom.

It should also be understood that, the term “methylene unit” or “carbon unit” can also refer to branched or substituted methylene or carbon units. For example, in an isopropyl moiety [—CH(CH₃)₂], a nitrogen atom (e.g., NR) replacing the first recited “methylene unit” would result in dimethylamine [—N(CH₃)₂]. In instances such as these, one of skill in the art would understand that the nitrogen atom will not have any additional atoms bonded to it, and the “R” from “NR” would be absent in this case.

Unless otherwise indicated, the optional replacements form a chemically stable compound. Optional replacements can occur both within the chain and/or at either end of the chain; i.e. both at the point of attachment and/or also at the terminal end. Two optional replacements can also be adjacent to each other within a chain so long as it results in a chemically stable compound. For example, a C₃ aliphatic can be optionally replaced by 2 nitrogen atoms to form —C—N≡N.

Unless otherwise indicated, if the replacement occurs at the terminal end, the replacement atom is bound to a hydrogen atom on the terminal end. For example, if a methylene unit of —CH₂CH₂CH₃ were optionally replaced with —O—, the resulting compound could be —OCH₂CH₃, —CH₂OCH₃, or —CH₂CH₂OH. It should be understood that if the terminal atom does not contain any free valence electrons, then a hydrogen atom is not required at the terminal end (e.g., —CH₂CH₂CH═O or —CH₂CH₂C≡N).

Unless otherwise indicated, structures depicted herein are also meant to include all isomeric (e.g., enantiomeric, diastereomeric, geometric, conformational, and rotational) forms of the structure. For example, the R and S configurations for each asymmetric center, (Z) and (E) double bond isomers, and (Z) and (E) conformational isomers are included in this invention. As would be understood to one skilled in the art, a substituent can freely rotate around any rotatable bonds. For example, a substituent drawn as

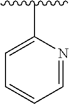 [SMILES: *c1ccccn1]

also represents

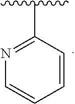 [SMILES: *c1ccccn1]

Therefore, single stereochemical isomers as well as enantiomeric, diastereomeric, geometric, conformational, and rotational mixtures of the present compounds are within the scope of the invention.

Unless otherwise indicated, all tautomeric forms of the compounds of the invention are within the scope of the invention.

In some aspects, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, for therapeutics and/or analytical tools or probes in biological assays. Especially deuterium (²H)-labeled compounds can also be used for therapeutic purposes.

In some embodiments, a provided chemical entity is an isotope-labeled chemical entity, which is an isotope-labeled free compound of Formula (I′), such as an isotope-labeled free compound of Formula (II′), (III′), (A′), (B′), (C′), (1′), (3′), (II.A′), (II.B′), (II.C′), (II.1′), (III.A′), (III.B′), (III.C′), (III.1′), (A.1′), (B.1′), (C.1′), (II.A.1′), (II.B.1′), (II.C.1′), (III.A.1′), (III.A.1a′), (III.A.1b′), (III.A.3′), (III.B.1′) and/or (III.C.1′), or a pharmaceutically acceptable salt thereof, wherein the formula and variables of the foregoing Formulas are each and independently as described above for Formula (I), (II), (III), (A), (B), (C), (1), (3), (II.A), (II.B), (II.C), (II.1), (III.A), (III.B), (III.C), (III.1), (A.1), (B.1), (C.1), (II.A.1), (II.B.1), (II.C.1), (III.A.1), (III.A.1a), (III.A.1b), (III.A.3), (III.B.1), (III.C.1), or any other embodiments described above, provided that one or more atoms therein have been replaced by an atom or atoms having an atomic mass or mass number which differs from the atomic mass or mass number of the atom which usually occurs naturally (“isotope labeled”). Examples of isotopes which are commercially available and suitable for the invention include isotopes of hydrogen, carbon, nitrogen, oxygen, phosphorus, fluorine and chlorine, for example, ²H, ³H, ¹³C, ¹⁴C, ¹⁵N, ¹⁸O, ¹⁷O, ³¹P, ³²P, ³⁵S, ¹⁸F and ³⁶Cl, respectively.

The isotope-labeled chemical entities of the invention (e.g., free compounds and pharmaceutically acceptable salts thereof) can be used in a number of beneficial ways. They can be suitable for medicaments and/or various types of assays, such as substrate tissue distribution assays. For example, tritium (³H)- and/or carbon-14 (¹⁴C)-labeled compounds are particularly useful for various types of assays, such as substrate tissue distribution assays, due to relatively simple preparation and excellent detectability. For example, deuterium (²H)-labeled compounds are therapeutically useful with potential therapeutic advantages over the non-²H-labeled compounds. In some instances, deuterium (²H)-labeled compounds can have higher metabolic stability as compared to those compounds that are not isotope-labeled owing to the kinetic isotope effect described below. Higher metabolic stability generally translates directly into an increased in vivo half-life or lower dosages, which under most circumstances would represent a preferred embodiment of the present invention. The isotope-labeled compounds of the invention can usually be prepared by carrying out the procedures described herein, replacing a non-isotope-labeled reactant by a readily available isotope-labeled reactant.

In some embodiments, the isotope-labeled compounds of the invention are deuterium (²H)-labeled compounds. In some embodiments, the invention is directed to deuterium (²H)-labeled chemical entities of Formula (I), such as chemical entities of Formula (II), (III), (A), (B), (C), (1), (3), (II.A), (II.B), (II.C), (II.1), (III.A), (III.B), (III.C), (III.1), (A.1), (B.1), (C.1), (II.A.1), (II.B.1), (II.C.1), (III.A.1), (III.A.1a), (III.A.1b), (III.A.3), (III.B.1) and/or (III.C.1). In some embodiments, the invention is directed to deuterium (²H)-labeled compounds of Table 1. In some embodiments, one, two, three or four hydrogen atoms are replaced by deuterium. In some embodiments, one hydrogen atom is replaced by deuterium. In some embodiments, two hydrogen atoms are replaced by deuterium. In some embodiments, three hydrogen atoms are replaced by deuterium. In some embodiments, four hydrogen atoms are replaced by deuterium.

Deuterium (²H)-labeled compounds of the invention can manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. If this rate difference is successfully applied to, for example, a compound of Formula (I′), the profile of this compound in vivo can be drastically modified and result in improved pharmacokinetic properties. For a further discussion, see S. L. Harbeson and R. D. Tung, Deuterium In Drug Discovery and Development, Ann. Rep. Med. Chem. 2011, 46, 403-417, incorporated in its entirety herein by reference.

The concentration of the isotope(s) (e.g., deuterium) incorporated into the isotope-labeled compounds of the invention may be defined by the isotopic enrichment factor. The term “isotopic enrichment factor” as used herein means the ratio between the isotopic abundance and the natural abundance of a specified isotope. In some embodiments, if a substituent in a compound of the invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

When discovering and developing therapeutic agents, the person skilled in the art attempts to maximize pharmacokinetic parameters while retaining desirable in vitro properties. In vitro liver microsomal assays currently available provide valuable information on the course of hepatic microsomal oxidative metabolism, which in turn permits the rational design of the deuterium (²H)-labeled compounds of the invention which can have improved stability through resistance to such oxidative metabolism. Significant improvements in the pharmacokinetic profiles of such compounds can thereby be obtained, and can be expressed quantitatively in terms of increases in the in vivo half-life (t_{1/2}), concentration at maximum therapeutic effect (Cₘₐₓ), area under the dose response curve (AUC), and bioavailability; and in terms of reduced clearance, dose and materials costs.

The following is intended to illustrate the above: a deuterium (²H)-labeled compound of the invention, which has multiple potential sites of attack for oxidative metabolism, for example benzylic hydrogen atoms and hydrogen atoms bonded to a nitrogen atom, is prepared as a series of analogues in which various combinations of hydrogen atoms are replaced by deuterium atoms, so that some, most or all of these hydrogen atoms have been replaced by deuterium atoms. Half-life determinations enable favorable and accurate determination of the extent to which the improvement in resistance to oxidative metabolism has improved. In this way, it is determined that the half-life of the parent compound can be extended by up to 100% as the result of deuterium-hydrogen exchange of this type.

Deuterium-hydrogen exchange in a deuterium (²H)-labeled compound of the invention can also be used to achieve a favorable modification of the metabolite spectrum of the starting compound in order to diminish or eliminate undesired toxic metabolites. For example, if a toxic metabolite arises through oxidative carbon-hydrogen (C—H) bond cleavage, the deuterated analogue may greatly diminish or eliminate production of the unwanted metabolite, even if the particular oxidation is not a rate-determining step. Further information on the state of the art with respect to deuterium-hydrogen exchange may be found, for example in Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al., Biochemistry 33(10) 2927-2937, 1994, and Jarman et al. Carcinogenesis 16(4), 683-688, 1993.

### Pharmacology

Adrenoleukodystrophy (ALD), also known as X-linked adrenoleukodystrophy or X-adrenoleukodystrophy (X-ALD), is a metabolic disorder in which patients accumulate VLCFA due to the absence or misfolding of ALD protein, a peroxisomal endoplasmic reticulum membrane protein encoded by the ATP Binding Cassette protein D1 (ABCD1) transporter gene. (Mosser, et al. Nature (1993), 361: 726-730) This transporter ALD protein is required for the import of VLCFA into peroxisomes where they are degraded through beta-oxidation by proteins including Acyl-CoA oxidase (ACOX1) and D-Bifunctional protein. VLCFA elongation occurs via the successive addition of 2 carbon atom units by ELOVL family members (Jakobsson A., et al. Prog. Lipid Res. 2006; 45:237-249). ELOVL6 elongates shorter VLCFA; ELOVL7 elongates mid-range VLCFA; and ELOVL1 is primarily responsible for the synthesis of C26:0 (T. Sassa, et al. J. Lipid Res. 55(3), (2014): 524-530). ALD is associated with impaired peroxisomal beta-oxidation and accumulation of very long-chain fatty acids (VLCFA) in tissues and body fluids (e.g., plasma, cerebrospinal fluid (CSF)). Mutations in the ABCD1 gene impair the degradation of VLCFA by preventing their transportation into peroxisomes where they are broken down by beta-oxidation. This disruption in the VLCFA degradation process results in the accumulation of VLCFA, for example, C24:0 and C26:0, in plasma and tissues. ALD patients accumulate C26:0 (and longer carbon chain lengths) VLCFA and their incorporation products, including lysophosphatidylcholines (LPC), sphingomyelins, acylcarnitines, cholesterol esters and ceramides. These accumulating VLCFA are thought to be particularly detrimental to the central nervous system; accumulation of C26:0 VLCFA are thought to be the pathological factor disrupting the fatty acid-rich myelin sheath, the adrenal glands and Leydig cells in testes; ABCD1 KO mice exhibit a thickening of myelin that appears to disrupt peripheral axons and leads to AMN-like symptoms. (A. Pujol et al., Human Molecular Genetics 2002, 11: 499-505). Interestingly, mutations in either Acyl-CoA oxidase or D-Bifunctional protein also lead to accumulation of VLCFA and fatal demyelinating disorders, supporting the hypothesis that increased VLCFA cause the underlying pathophysiology of ALD.

High levels of C26:0 have been correlated with pathogenic effects. (R. Orfman et al., EMBO Mol. Med. 2010, 2:90-97). For example, C26:0 decreases the response of adrenocortical cells to adrenocorticotropic hormone stimulation. (R. W. Whitcomb et al., J. Clin. Invest. 1988, 81:185-188). A pathogenic role for C26:0 is further supported by its disruptive effects on the structure, stability and function of cell membranes (J. K. Ho et al., J. Clin. Invest. 1995, 96:1455-1463; R. A. Knazek et al., J. Clin. Invest. 1983, 72:245-248), and by its possible contribution to oxidative stress. (S. Fourcade et al., Hum. Mol. Genet. 2008, 17:1762-1773; J. M. Powers et al., J. Neuropathol. Exp. 2005, 64:1067-1079).

Mutations in other proteins of the VLCFA degradation pathway, Acyl-CoA oxidase, D-Bifunctional protein (DBP), Acyl-CoA binding domain containing protein 5 (ACBD5), also contribute to VLCFA accumulation and demyelinating diseases in humans.

In some embodiments, the chemical entities are useful for treating at least one of the following diseases: ALD and its phenotypes (e.g., CALD and AMN), ACOX deficiency, DBP deficiency, ACBD5 deficiency, or Zellweger spectrum disorders (ZSDs).

VLCFA are synthesized by the fatty acid elongation cycle, and the rate-limiting step is enzymatically catalyzed by the elongation of very long-chain fatty acids (ELOVL). Of the seven known ELOVL isozymes, ELOVL1 is the primary enzyme responsible for the synthesis of C22:0 to C26:0 VLCFA that are accumulated in ALD patients. (Orfman). Accordingly, compounds that inhibit ELOVL1 may be useful in suppressing the synthesis of VLCFA and therefore useful in the treatment of disorders such as ALD. Without being bound by theory, certain compounds described herein, such as Compound 87, inhibit ELOVL1, which may cause the reduction in VLCFA levels observed herein.

### Pharmaceutically Acceptable Salts

The compounds of this invention can exist in free form for treatment, or where appropriate, as a pharmaceutically acceptable salt.

A “pharmaceutically acceptable salt” means any non-toxic salt of a chemical entity described herein that, upon administration to a patient or to a sample, is capable of providing, either directly or indirectly, the chemical entity or an active metabolite or residue thereof. As used herein, the term “active metabolite or residue thereof” means that a metabolite or residue thereof also provides a reduction in a VLCFA level.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified free compound in its free-base form with a suitable organic or inorganic acid and 2) isolating the salt thus formed.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Base addition salts can be prepared by 1) reacting the purified free compound in its free acid form with a suitable organic or inorganic base and 2) isolating the salt thus formed. Salts derived from appropriate bases include alkali metal (e.g., sodium, lithium, and potassium), alkaline earth metal (e.g., magnesium and calcium), ammonium and N+(C₁₋₄ alkyl)₄ salts. This invention also envisions the quaternization of any basic nitrogen-containing groups of the compounds disclosed herein. Water or oil-soluble or dispersible products may be obtained by such quaternization.

Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

### Pharmaceutically Acceptable Derivatives or Prodrugs

In addition to the compounds of this invention, pharmaceutically acceptable derivatives or prodrugs of the compounds of this invention may also be employed in compositions to treat or prevent the diseases, conditions and disorders. Specific examples are described below.

The compounds of this invention can also exist as pharmaceutically acceptable derivatives. A “pharmaceutically acceptable derivative” is an adduct or derivative which, upon administration to a patient in need, is capable of providing, directly or indirectly, a compound as otherwise described herein, or a metabolite or residue thereof. Examples of pharmaceutically acceptable derivatives include esters and salts of such esters.

A “pharmaceutically acceptable derivative or prodrug” means any pharmaceutically acceptable ester, salt of an ester or other derivative or salt thereof of a chemical entity described herein that upon administration to a patient or sample, is capable of providing, either directly or indirectly, the chemical entity or an active metabolite or residue thereof. Particularly favored derivatives or prodrugs are those that increase the bioavailability of a chemical entity described herein when such chemical entity is administered to a patient (e.g., by allowing an orally administered compound to be more readily absorbed into the blood) or sample, or which enhance delivery of the chemical entity to a biological compartment (e.g., the brain or lymphatic system), tissue, biological fluid or cell relative to the chemical entity that is not delivered as a derivative or prodrug.

Pharmaceutically acceptable prodrugs of the compounds of this invention include esters, amino acid esters, phosphate esters, metal salts and sulfonate esters.

### Pharmaceutical Compositions

The present invention also provides chemical entities and compositions that are useful for reduction of VLCFA levels or for treating disorders related to impaired peroxisomal function (e.g., impaired transport of VLCFA into the peroxisomes or impaired VLCFA degradation/metabolism within the peroxisomes) or accumulation of very long-chain fatty acids (VLCFA).

In some aspects the present invention provides pharmaceutically acceptable compositions that comprise any of the chemical entities as described herein, and additionally comprise a pharmaceutically acceptable carrier, adjuvant or excipient.

The pharmaceutically acceptable carrier, adjuvant, or excipient, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. REMINGTON: THE SCIENCE AND PRACTICE OF PHARMACY, 20^{th} Edition, A. R. Gennaro (ed.), Lippincott Williams & Wilkins: Baltimore, Md. (2000) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention.

Some examples of materials which can serve as pharmaceutically acceptable carriers include ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate, sodium stearyl fumarate, and magnesium stearate, as well as coloring agents, releasing agents, coating agents, sweetening, flavoring and perfuming agents, preservatives and antioxidants can also be present in the composition, according to the judgment of the formulator.

The chemical entities of the invention can be formulated into pharmaceutical compositions for administration to animals or humans. In some embodiments, these pharmaceutical compositions comprise an amount of a chemical entity described herein effective to treat or prevent the diseases or conditions described herein and a pharmaceutically acceptable carrier, adjuvant, or excipient.

The exact amount of compound required for treatment will vary from subject to subject, depending on the species, age, and general condition of the subject, the severity of the disease, the particular agent, its mode of administration, and the like. The chemical entities of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

In some embodiments, these compositions optionally further comprise one or more additional therapeutic agents. Some embodiments provide a simultaneous, separate or sequential use of a combined preparation.

### Uses and Methods of Treatment

In some aspects, the present invention provides chemical entities that reduce a VLCFA level and compositions comprising such chemical entities, as described above. In some aspects, the present invention provides methods and uses for treating or preventing a disease, condition, or disorder responsive to reduction in VLCFA level, which employ administering a chemical entity of the invention, such as a compound of Formula I or a pharmaceutically acceptable salt thereof, or a pharmaceutical composition of the invention comprising such chemical entity. Such methods and uses typically employ administering an effective amount of a chemical entity or pharmaceutical composition of the invention to a patient or subject. In some embodiments, the reduction in VLCFA level is reversible.

The terms, “disease”, “disorder”, and “condition” may be used interchangeably herein to refer to any deviation from or interruption of the normal structure or function of any body part, organ, or system that is manifested by a characteristic set of symptoms and signs. Diseases, disorders and conditions of particular interest in the context of the present invention are those responsive to reduction of VLCFA level.

As used herein, the terms “subject” and “patient” are used interchangeably. The terms “subject” and “patient” refer to an animal (e.g., a bird such as a chicken, quail or turkey, or a mammal), particularly a mammal including non-primates (e.g., a cow, pig, horse, sheep, rabbit, guinea pig, rat, cat, dog, or mouse) and primates (e.g., a monkey, chimpanzee or human), and more particularly a human. In some embodiments, the subject is a non-human animal such as a farm animal (e.g., a horse, cow, pig or sheep), or a pet (e.g., a dog, cat, guinea pig or rabbit). In some embodiments, the subject is a human.

As used herein, an “effective amount” refers to an amount sufficient to elicit the desired biological response. In the present invention, certain examples of the desired biological response is to treat or prevent a disease, condition or disorder responsive to reduction in VLCFA level, or to enhance or improve the prophylactic or therapeutic effect(s) of another therapy used against a disease, condition or disorder responsive to reduction in VLCFA level. The precise amount of compound administered to a subject will depend on the mode of administration, the type and severity of the disease, condition, or disorder and on the characteristics of the patient, such as general health, age, sex, body weight and tolerance to drugs. Persons skilled in the art will be able to determine appropriate dosages depending on these and other factors. When co-administered with other agents, an “effective amount” of the second agent will depend on the type of drug used. Suitable dosages are known for approved agents and can be adjusted by the person skilled in the art according to the condition of the patient, the type of condition(s) being treated and the amount of a compound described herein being used. For example, chemical entities described herein can be administered to a subject in a dosage range from between approximately 0.01 to 100 mg/kg body weight/day for therapeutic or prophylactic treatment. The chemical entities and compositions, according to the methods of the present invention, may be administered using any amount and any route of administration effective for eliciting the desired biological response.

As used herein, the terms “treat,” “treatment” and “treating” can refer to both therapeutic and prophylactic treatments. For example, therapeutic treatments include the reduction, amelioration, slowing or arrest of the progression, severity and/or duration of one or more conditions, diseases or disorders and/or of one or more symptoms (specifically, one or more discernible symptoms) thereof, resulting from the administration of one or more therapies (e.g., one or more therapeutic agents such as a chemical entity or composition of the invention). In some embodiments, treatment refers to reduction or amelioration of the progression, severity and/or duration of one or more conditions, diseases or disorders, resulting from the administration of one or more therapies. In some embodiments, treatment refers to reduction or amelioration of the severity and/or duration of one or more conditions, diseases or disorders, resulting from the administration of one or more therapies. In some embodiments, treatment refers to reduction or amelioration of the progression, severity and/or duration of one or more symptoms (specifically, one or more discernible symptoms) of one or more conditions, diseases or disorders, resulting from the administration of one or more therapies. In some embodiments, treatment refers to reduction or amelioration of the severity and/or duration of one or more symptoms (specifically, one or more discernible symptoms) of one or more conditions, diseases or disorders, resulting from the administration of one or more therapies. Prophylactic treatments include prevention or delay of the onset of one or more conditions, diseases or disorders and/or of one or more symptoms (specifically, one or more discernible symptoms) thereof, resulting from the administration of one or more therapies (e.g., one or more therapeutic agents such as a chemical entity or composition of the invention). In some embodiments, treatment refers to prevention or delay of the onset of one or more conditions, diseases or disorders resulting from the administration of one or more therapies. In some embodiments, treatment refers to prevention or delay of the onset of one or more symptoms (specifically, one or more discernible symptoms) of one or more conditions, diseases or disorders resulting from the administration of one or more therapies.

In some embodiments, the invention provides co-administering to a patient an additional therapeutic agent, wherein said additional therapeutic agent is appropriate for the disease, condition or disorder being treated; and said additional therapeutic agent is administered together with a chemical entity of the invention as a single dosage form, or separately from said compound as part of a multiple dosage form.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a patient, nor does it require administration in any specific proximity in time, so long as in the judgment of a suitable physician the patient is understood to be receiving the one or more therapies at the same time. For example, receiving therapy A on days 1-5 of a 28-day schedule and therapy B on days 1, 8 and 15 of a 21-day schedule would be considered “in combination” or a “co-administration”.

Co-administration also encompasses administration of the first and second amounts of the compounds of the co-administration in an essentially simultaneous manner, such as in a single pharmaceutical composition, for example, capsule or tablet having a fixed ratio of first and second amounts, or in multiple, separate capsules or tablets for each. In addition, such co-administration also encompasses use of each compound in a sequential manner in either order.

Therapies which may be used in combination with the chemical entities of the present invention include Lorenzo's Oil (4:1 glycerol trioleate and glyceryl trierucate), allogenic hematopoetic stem cell transplant, autologous hematopoetic stem cell transplant, corticosteroid replacement therapy and CNS gene replacement therapy.

### Modes of Administration and Dosage Forms

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray or via inhalation, or the like, depending on the identity and/or severity of the disease being treated. In certain embodiments, the chemical entities of the invention may be administered orally or parenterally at dosage levels of about 0.01 mg/kg to about 50 mg/kg, about 0.1 mg/kg to about 50 mg/kg, of subject body weight per day, one or more times a day, to obtain the desired therapeutic effect.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), derivatized/modified beta-cyclodextrin, glycerol, tetrahydrofurfuryl alcohol, polyethylene glycols and fatty acid esters of sorbitan, sodium lauryl sulfate, d-α-tocopheryl polyethylene glycol succinate (TPGS; also called vitamin E-TPGS or tocophersolan), and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents (or disintegrant) such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as d-α-tocopheryl polyethylene glycol succinate (TPGS; also called vitamin E-TPGS or tocophersolan), Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavouring or colouring agents may also be added.

Alternatively, the pharmaceutical compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of chemical entity that may be combined with the carrier materials to produce a single dosage form will vary depending upon the host treated, the particular mode of administration. Preferably, the compositions should be formulated so that a dosage of between 0.01-100 mg/kg body weight/day of the chemical entity can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of the chemical entity will also depend upon the particular compound in the composition.

Administering with another Agent

Depending upon the particular conditions to be treated or prevented, additional drugs, which are normally administered to treat or prevent that condition, may be administered together with the chemical entities of this invention.

Those additional agents may be administered separately, as part of a multiple dosage regimen. Alternatively, those agents may be part of a single dosage form, mixed together with the chemical entity in a single composition.

### Biological Samples

The chemical entities and compositions of this invention are also useful in biological samples. In some aspects, the invention relates to a reduction in VLCFA level in a biological sample, which method comprises contacting said biological sample with a chemical entity described herein or a composition comprising said chemical entity. The term “biological sample”, as used herein, means an in vitro or an ex vivo sample, including cell cultures or extracts thereof; biopsied material obtained from a mammal or extracts thereof; and blood, saliva, urine, feces, semen, tears, or other body fluids or extracts thereof. The term “chemical entities described herein” includes chemical entities of Formula I.

### Synthetic Methods

In general, the chemical entities of the invention can be prepared by methods described herein or by other methods known to those skilled in the art. Exemplary preparations of the chemical entities of the invention are described below.

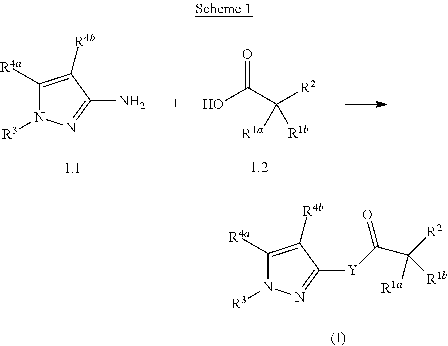

An exemplary synthetic route to compounds of Formula (I) is shown above in Scheme 1. Compounds listed in Table A can be made, for example, via this route. Amine 1.1 and carboxylic acid 1.2 can be coupled using amide bond-forming methods known in the art such as Methods A through R described below for Scheme Amide-1.

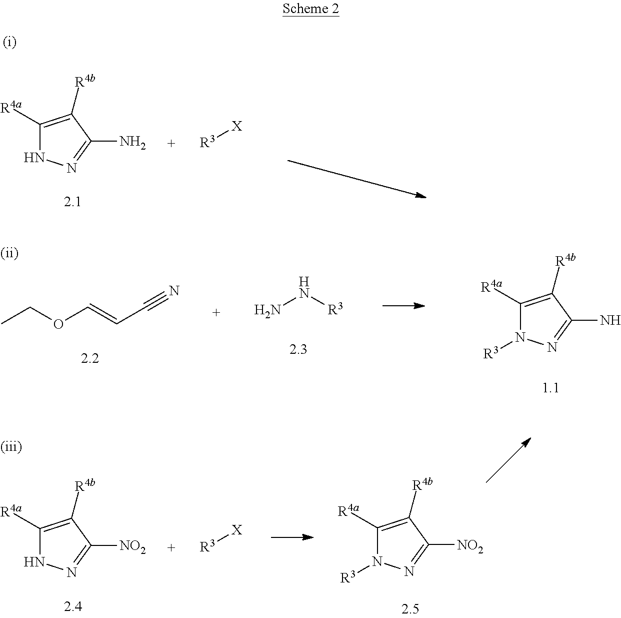

Exemplary synthetic routes to amine 1.1 are shown above in Scheme 2. For example, (i) pyrazole 2.1 can be coupled to halide R³—X using methods known in the art such as copper bromide-mediated coupling as described below for Scheme Amine-2. Alternatively, (ii) nitrile 2.2 can be reacted with hydrazine 2.3 under conditions known in the art suitable to form amine 1.1, e.g., those described below for Scheme Amine-3. In another alternative synthesis (iii) nitro-substituted pyrazole 2.4 can be coupled to halide R³—X and then reduced using methods known in the art, e.g., those described below for Scheme Amine-4.

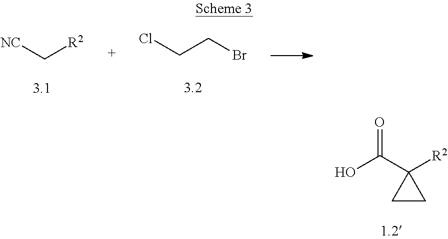

An exemplary synthetic route to carboxylic acid 1.2 is shown above in Scheme 3. Nitrile 3.1 can be reacted with an appropriate electrophile 3.2 using methods known in the art suitable to form carboxylic acid 1.2, e.g., those described below for Scheme Acid-1. Scheme 3 illustrates the formation of cyclopropane carboxylic acid 1.2′; however, suitable selection of electrophile 3.2 and appropriate modification to make other carboxylic acids 1.2 will be apparent to persons skilled in the art.

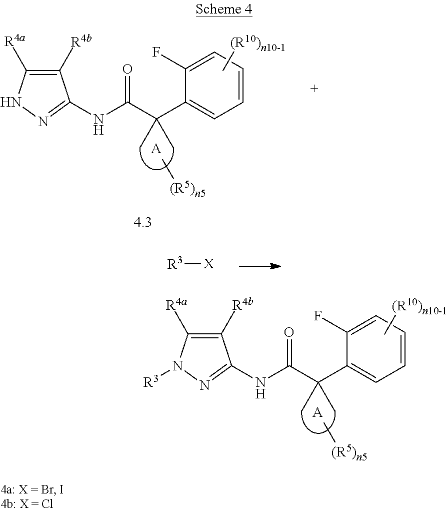

An alternative synthetic route to compounds of Formula (I) is shown above in Scheme 4. Compounds listed in Tables B and C can be made, for example, via routes (4a) and (4b), respectively. Pyrazole 4.3 can be coupled to halide R³—X using methods known in the art such as copper-mediated coupling Methods A through C described below for Scheme Aryl-2 when X is Br or I, or nucleophilic displacement as described below for Scheme S_{N}Ar-1 when X is Cl.

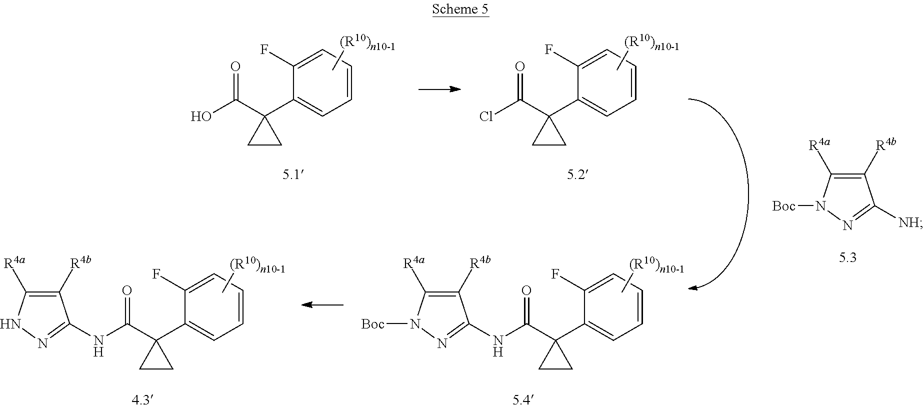

An exemplary synthetic route to pyrazole 4.3 is shown above in Scheme 5. Carboxylic acid 5.1 can be converted to the corresponding acid chloride 5.2 and coupled to 1H-protected pyrazolamine 5.3 followed by deprotection to pyrazole 4.3 using methods known in the art such as those described below for Scheme Aryl-1. Scheme 5 illustrates the formation of cyclopropane- and phenyl-containing pyrazole 4.3′ starting from carboxylic acid 5.1′; however, suitable selection of cyclopropane carboxylic acid 5.1 and appropriate modification to make other pyrazoles 4.3 will be apparent to persons skilled in the art.

### Enumerated Embodiments

In some embodiments, provided are:

1. a. A chemical entity, which is a free compound of formula (I) or a pharmaceutically acceptable salt thereof, wherein Formula (I) has the structure,

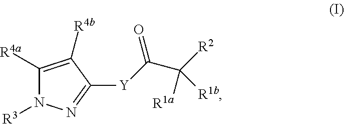

each of R^{1a} and R^{1b} independently is H, —C₁₋₄ alkyl, C₁₋₄ haloalkyl, —(C(R^{J1a}₂))₁₋₂—OH, —(C(R^{J1a}₂))₁₋₂—OR^{J1}, —(C(R^{J1a}₂))₁₋₂—SR^{J}, —(C(R^{J1a}₂))₁₋₂—NH₂, —(C(R^{J1a}₂))₁₋₂—NHR^{J1}, —(C(R^{J1a}₂))₁₋₂—NR^{J1}₂, C₃₋₆ cycloalkyl or a 3- to 6-membered monocyclic heterocycle containing 1 ring heteroatom selected from O, N, and S,
wherein the 3- to 6-membered monocyclic heterocycle does not contain a heteroatom bonded to the carbon to which R^{1a} and R^{1b} are attached,
wherein each instance of R^{J1} is independently C₁₋₃ alkyl or C₁₋₄ haloalkyl,
wherein each instance of R^{J1a} is independently H, C₁₋₃ alkyl, C₁₋₄ haloalkyl;
or
R^{1a} and R^{1b}, together with the carbon atom to which they are attached form a C₃₋₆cycloalkyl, or a 3- to 6-membered monocyclic heterocycle containing 1 ring heteroatom selected from O, N and S, wherein the 1 ring heteroatom is not bonded to the carbon to which R^{1a} and R^{1b} are attached;
wherein each of said C₃₋₆ cycloalkyl and said 3- to 6-membered monocyclic heterocycle is unsubstituted or substituted with 1 or 2 substituents independently selected from halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, (C(R^{J1a}₂))₀₋₂—NH₂, —(C(R^{J1a}₂))₀₋₂—NHR^{J1}, and —(C(R^{J1a}₂))₀₋₂—NR^{J1}₂, or wherein two geminal substituents, together with the carbon atom to which they are attached, form a C₃₋₆ cycloalkyl or 3- to 6-membered monocyclic heterocycle containing 1-2 heteroatoms selected from O, N, and S,
wherein each instance of R^{J1} is independently C₁₋₃ alkyl or C₁₋₄ haloalkyl,
wherein each instance of R^{J1a} is independently H, C₁₋₃ alkyl, or C₁₋₄ haloalkyl;
R² is phenyl or 5- or 6-membered monocyclic heteroaryl having 1-3 ring heteroatoms independently selected from O, N and S,
wherein each of said phenyl and said 5- or 6-membered monocyclic heteroaryl is unsubstituted or substituted with 1-3 substituents independently selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J2a}₂))₀₋₂—SR^{J2}, —(C(R^{J2a}₂))₀₋₂—NH₂, —(C(R^{J2a}₂))₀₋₂—NHR^{J2}, —(C(R^{J2a}₂))₀₋₂—NR^{J2}₂, —C(O)R^{J1}₂, and —CN,
wherein each instance of R^{J2} is independently C₁₋₃ alkyl or C₁₋₄ haloalkyl,
wherein each instance of R^{J2a} is independently H, C₁₋₃ alkyl, or C₁₋₄ haloalkyl,
wherein optionally methylenedioxy constitutes a substituent of said phenyl, wherein the methylene unit of the methylenedioxy is unsubstituted or substituted with halo; and
R³ is phenyl, or 5- or 6-membered monocyclic heteroaryl having 1-4 ring heteroatoms independently selected from O, N and S,
wherein each of said phenyl and said 5- or 6-membered monocyclic heteroaryl is unsubstituted or substituted with 1-3 substituents independently selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, or C₁₋₄ haloalkyl;
each of R^{4a} and R^{4b} independently is —H, halo, C₁₋₄ alkyl and
Y is —NH— or —N(C₁₋₄ alkyl)-;
wherein 0 to 6 hydrogen atoms of said compound of Formula (I) are optionally replaced with deuterium; provided that the compound of Formula (I) is not

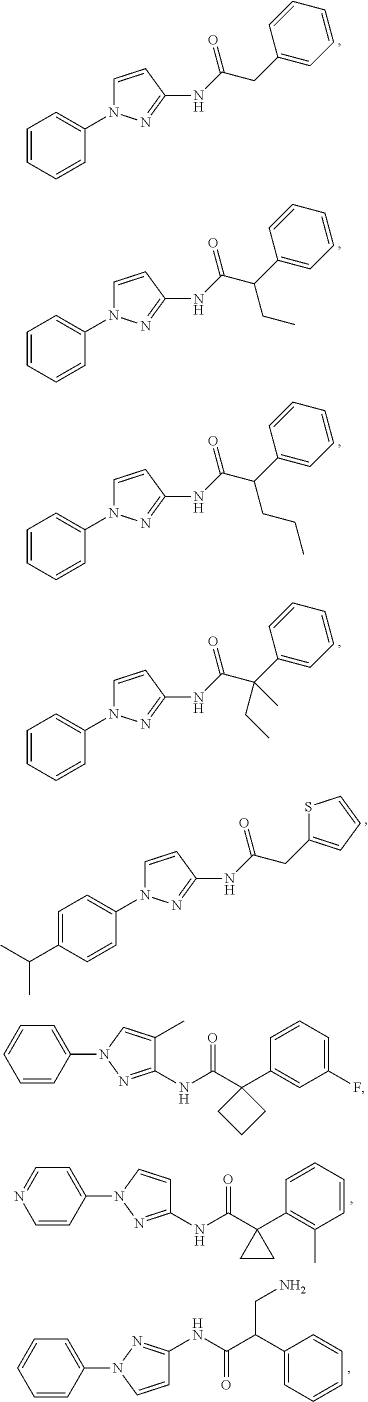

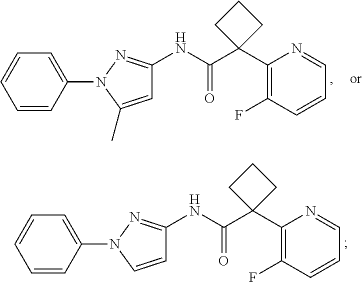 [SMILES: Cc1cc(NC(=O)C2(c3ncccc3F)CCC2)nn1-c1ccccc1 ; O=C(Nc1ccn(-c2ccccc2)n1)C1(c2ncccc2F)CCC1]

or

b. A chemical entity, which is a free compound of formula (I) or a pharmaceutically acceptable salt thereof, wherein Formula (I) has the structure,

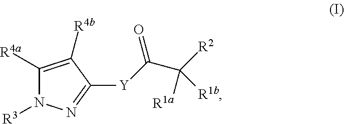

each of R^{1a} and R^{1b} independently is H, —C₁₋₄ alkyl, C₁₋₄ haloalkyl, —(C(R^{J1a}₂))₁₋₂—OH, —(C(R^{J1a}₂))₁₋₂—OR^{J1}, —(C(R^{J1a}₂))₁₋₂—SR^{J1}, —(C(R^{J1a}₂))₁₋₂—NH₂, —(C(R^{J1a}₂))₁₋₂—NHR^{J1}, —(C(R^{J1a}₂))₁₋₂—NR^{J1}₂, C₃₋₆ cycloalkyl or a 3- to 6-membered monocyclic heterocycle containing 1 ring heteroatom selected from O, N, and S,
wherein the 3- to 6-membered monocyclic heterocycle does not contain a heteroatom bonded to the carbon to which R^{1a} and R^{1b} are attached,
wherein each instance of R^{J1} is independently C₁₋₃ alkyl or C₁₋₄ haloalkyl,
wherein each instance of R^{J1a} is independently H, C₁₋₃ alkyl, C₁₋₄ haloalkyl;
or
R^{1a} and R^{1b}, together with the carbon atom to which they are attached form a C₃₋₆cycloalkyl, or a 3- to 6-membered monocyclic heterocycle containing 1 ring heteroatom selected from O, N and S, wherein the 1 ring heteroatom is not bonded to the carbon to which R^{1a} and R^{1b} are attached;
wherein each of said C₃₋₆ cycloalkyl and said 3- to 6-membered monocyclic heterocycle is unsubstituted or substituted with 1 or 2 substituents independently selected from halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, (C(R^{J1a}₂))₀₋₂—NH₂, —(C(R^{J1a}₂))₀₋₂—NHR^{J1}, and —(C(R^{J1a}₂))₀₋₂—NR^{J1}₂, or wherein two geminal substituents, together with the carbon atom to which they are attached, form a C₃₋₆ cycloalkyl or 3- to 6-membered monocyclic heterocycle containing 1-2 heteroatoms selected from O, N, and S,
wherein each instance of R^{J1} is independently C₁₋₃ alkyl or C₁₋₄ haloalkyl,
wherein each instance of R^{J1a} is independently H, C₁₋₃ alkyl, or C₁₋₄ haloalkyl;
R² is phenyl or 5- or 6-membered monocyclic heteroaryl having 1-3 ring heteroatoms independently selected from O, N and S,
wherein each of said phenyl and said 5- or 6-membered monocyclic heteroaryl is unsubstituted or substituted with 1-3 substituents independently selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J2}, and —CN,
wherein each instance of R^{J2} is independently C₁₋₃ alkyl or C₁₋₄ haloalkyl,
wherein each instance of R^{J2a} is independently H, C₁₋₃ alkyl, or C₁₋₄ haloalkyl,
wherein optionally methylenedioxy constitutes a substituent of said phenyl, wherein the methylene unit of the methylenedioxy is unsubstituted or substituted with halo; and
R³ is phenyl, or 5- or 6-membered monocyclic heteroaryl having 1-4 ring heteroatoms independently selected from O, N and S,
wherein each of said phenyl and said 5- or 6-membered monocyclic heteroaryl is unsubstituted or substituted with 1-3 substituents independently selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, or C₁₋₄ haloalkyl;
each of R^{4a} and R^{4b} independently is —H, halo, C₁₋₄ alkyl and
Y is —NH— or —N(C₁₋₄ alkyl)-;
wherein 0 to 6 hydrogen atoms of said compound of Formula (I) are optionally replaced with deuterium; provided that the compound of Formula (I) is not

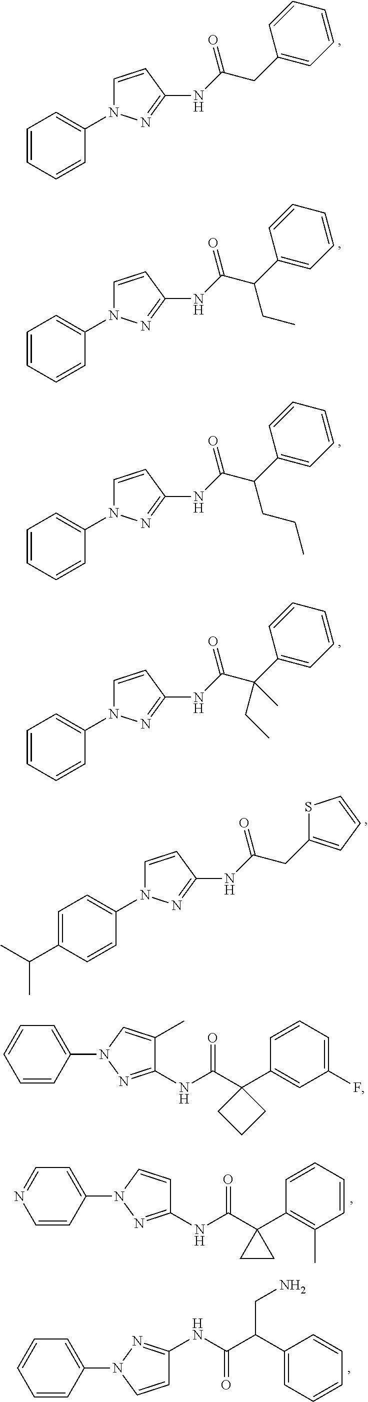

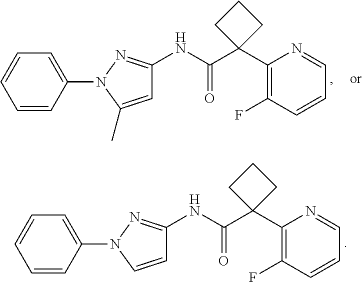 [SMILES: Cc1cc(NC(=O)C2(c3ncccc3F)CCC2)nn1-c1ccccc1 ; O=C(Nc1ccn(-c2ccccc2)n1)C1(c2ncccc2F)CCC1]

2. The chemical entity of embodiment 1, wherein each of R^{1a} and R^{1b} independently is H, —C₁₋₄ alkyl, C₁₋₄ haloalkyl, —(C(R^{J1a}₂))₁₋₂—OH, —(C(R^{J1a}₂))₁₋₂—OR^{J1}, —(C(R^{J1a}₂))₁₋₂—SR¹, —(C(R^{J1a}₂))₁₋₂—NH₂, —(C(R^{J1a}₂))₁₋₂—NHR^{J1}, —(C(R^{J1a}₂))₁₋₂—NR^{J1}₂, C₃₋₆ cycloalkyl or a 3- to 6-membered monocyclic heterocycle containing 1 ring heteroatom selected from O, N, and S,
wherein the 3- to 6-membered monocyclic heterocycle does not contain a heteroatom bonded to the carbon to which R^{1a} and R^{1b} are attached,
wherein each instance of R^{J1} is independently C₁₋₃ alkyl or C₁₋₄ haloalkyl,
wherein each instance of R^{J1a} is independently H, C₁₋₃ alkyl, C₁₋₄ haloalkyl;
or
R^{1a} and R^{1b}, together with the carbon atom to which they are attached form a C₃₋₆cycloalkyl, or a 3- to 6-membered monocyclic heterocycle containing 1 ring heteroatom selected from O, N and S, wherein the 1 ring heteroatom is not bonded to the carbon to which R^{1a} and R^{1b} are attached;
wherein each of said C₃₋₆ cycloalkyl and said 3- to 6-membered monocyclic heterocycle is unsubstituted or substituted with 1 or 2 substituents independently selected from halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, (C(R^{J1a}₂))₀₋₂—NH₂, —(C(R^{J1a}₂))₀₋₂—NHR^{J1}, and —(C(R^{J1a}₂))₀₋₂—NR^{J1}₂, or wherein two geminal substituents, together with the carbon atom to which they are attached, form a C₄₋₆ cycloalkyl or 4- to 6-membered monocyclic heterocycle containing 1-2 heteroatoms selected from O, N, and S.
3. The chemical entity of embodiment 1 or 2, which is a free compound of Formula (I).
4. The chemical entity of embodiment 1 or 2, which is a pharmaceutically acceptable salt of a compound of Formula (I).
5. The chemical entity of any one of embodiments 1-4, which is a chemical entity of Formula (II):

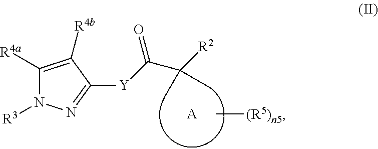

wherein:
A is a C₃₋₆ cycloalkyl or a 4- to 6-membered monocyclic heterocycle containing 1 ring heteroatom selected from O, N and S; wherein the 1 ring heteroatom is not bonded to the carbon to which A is attached;
each instance of R⁵ independently is selected from halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, —(C(R^{J1a}₂))₀₋₂—NH2, —(C(R^{J1a}₂))₀₋₂—NHR^{J1}, and —(C(R^{J1a}₂))₀₋₂—NR^{J1}₂ or two geminal R⁵, together with the carbon atom to which they are attached, form a C₃₋₆ cycloalkyl or 3- to 6-membered monocyclic heterocycle containing 1-2 heteroatoms selected from O, N, and S;
n5 is 0, 1 or 2.
6. The chemical entity of embodiment 5, wherein A is cyclopropyl, cyclobutyl or oxetanyl.
7. The chemical entity of any one of embodiments 1-4, which is a chemical entity of Formula (III):

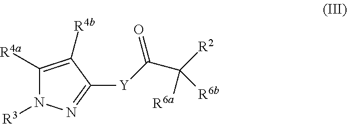

wherein:
each of R^{6a} and R^{6b} independently is —H, C₁₋₄ alkyl, C₁₋₄ haloalkyl, —(C(R^{J1a}₂))₁₋₂—OH, —(C(R^{J1a}₂))₁₋₂—OR^{J1}, —(C(R^{J1a}₂))₁₋₂—SR^{J1}, —(C(R^{J1a}₂))₁₋₂—NH₂, —(C(R^{J1a}₂))₁₋₂—NHR^{J1}, —(C(R^{J1a}₂))₁₋₂—NR^{J1}₂, C₃₋₆ cycloalkyl, or a 3- to 6-membered heterocycle containing 1 ring heteroatom selected from O, N, and S,
wherein the 3- to 6-membered monocyclic heterocycle does not contain a heteroatom bonded to the carbon to which R^{1a} and R^{1b} are attached,
wherein each instance of R^{J1} is independently C₁₋₃ alkyl or C₁₋₄ haloalkyl,
wherein each instance of R^{J1a} is independently H, C₁₋₃ alkyl, C₁₋₄ haloalkyl.
8. a. The chemical entity of any one of embodiments 1-4, which is a chemical entity of Formula (A):

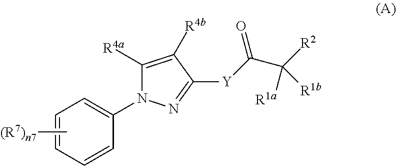

wherein:
each instance of R⁷ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, (C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl; and
n7 is 0, 1, 2 or 3; or

b. The chemical entity of any one of embodiments 1-4, which is a chemical entity of Formula (A):

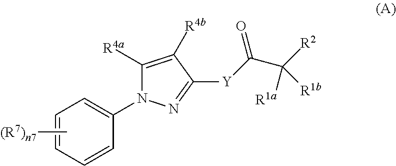

wherein:
each instance of R⁷ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR³², —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl; and
n7 is 0, 1, 2 or 3.
9. a. The chemical entity of any one of embodiments 1-4, which is a chemical entity of Formula (B):

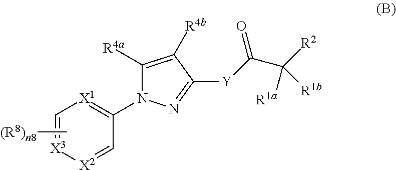

wherein:
one of X¹, X² and X³ is N, and the other two are carbon atoms;
each instance of R⁸ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, or C₁₋₄ haloalkyl; and
n8 is 0, 1, 2 or 3; or

b. The chemical entity of any one of embodiments 1-4, which is a chemical entity of Formula (B):

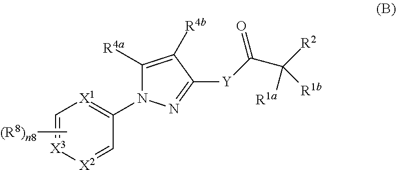

wherein:
one of X¹, X² and X³ is N, and the other two are carbon atoms;
each instance of R⁸ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, or C₁₋₄ haloalkyl; and
n8 is 0, 1, 2 or 3.
10. The chemical entity of embodiment 9, wherein X¹ is N, and X² and X³ are carbon atoms.
11. The chemical entity of embodiment 9, wherein X² is N, and X¹ and X³ are carbon atoms.
12. The chemical entity of embodiment 9, wherein X³ is N, and X¹ and X² are carbon atoms.
13. The chemical entity of embodiment 9, wherein:
one of X¹, X² and X³ is N, and the other two are carbon atoms such that
(a) when X¹ is N, then

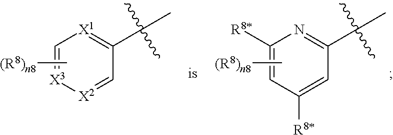

(b) when X² is N, then

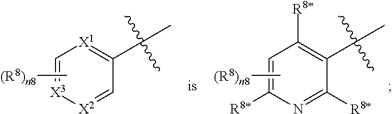

and
(c) when X³ is N, then

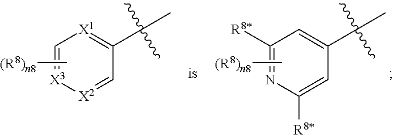

one instance of R⁸* is —F, and each of the other instances of R⁸* independently is —H, —F or R⁸;
each instance of R⁸ independently is selected from —Cl, —Br, —I, C₁₋₄ alkyl, C₁₋₄ haloalkyl, (C(R^{J3a}₂))₀₋₂—OH, —(C(R^{J3a}₂))₀₋₂—OR^{J3}, —NH₂, —NHR^{J3}, —N(R^{J3})₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, or C₁₋₄ haloalkyl;
n8* is equal to the number of instances of R⁸* that are not —H;
n8 is 0, 1 or 2 such that n8+n8*≤3,
14. a. The chemical entity of any one of embodiments 1-4, which is a chemical entity of Formula (C):

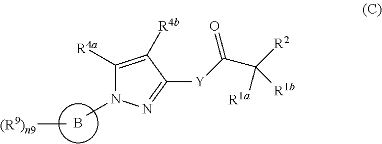

wherein:
B is 5-membered monocyclic heteroaryl having 1-4 ring heteroatoms independently selected from O, N and S, or 6-membered monocyclic heteroaryl having 2 or 3 ring nitrogen atoms;
each instance of R⁹ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR³², —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl; and
n9 is 0, 1, 2 or 3; or

b. The chemical entity of any one of embodiments 1-4, which is a chemical entity of Formula (C):

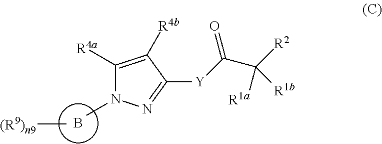

wherein:
B is 5-membered monocyclic heteroaryl having 1-4 ring heteroatoms independently selected from O, N and S, or 6-membered monocyclic heteroaryl having 2 or 3 ring nitrogen atoms;
each instance of R⁹ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl; and
n9 is 0, 1, 2 or 3.
15. a. The chemical entity of any one of embodiments 1-4, which is a chemical entity of Formula (1):

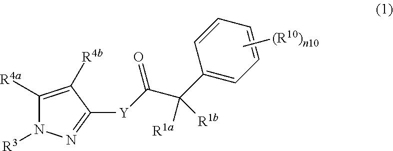

wherein:
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J2a}₂))₀₋₂—SR^{J2}, (C(R^{J2a}₂))₀₋₂—NH₂, —(C(R^{J2a}₂))₀₋₂—NHR^{J2}, (C(R^{J2a}₂))₀₋₂—NR^{J2}₂, —C(O)R^{J2}, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, or C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3; or

b. The chemical entity of any one of embodiments 1-4, which is a chemical entity of Formula (1):

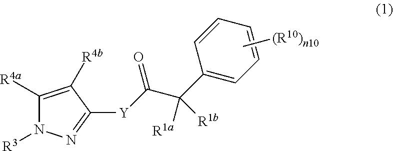

wherein:
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2} (C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J2}, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, or C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3.
16. a. The chemical entity of any one of embodiments 1-4, which is a chemical entity of Formula (3):

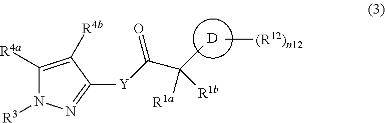

wherein:
D is 5- or 6-membered monocyclic heteroaryl having 1-3 ring heteroatoms independently selected from O, N and S;
each instance of R¹² independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J2a}₂))₀₋₂—SR^{J2}, (C(R^{J2a}₂))₀₋₂—NH₂, —(C(R^{J2a}₂))₀₋₂—NHR^{J2}, (C(R^{J2a}₂))₀₋₂—NR^{J2}₂, —C(O)R^{J2}, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, or C₁₋₄ haloalkyl,
wherein each of said C₅₋₇ carbocycle and said 5- to 7-membered monocyclic heterocycle is unsubstituted or substituted with halo; and
n12 is 0, 1, 2 or 3; or

b. The chemical entity of any one of embodiments 1-4, which is a chemical entity of Formula (3):

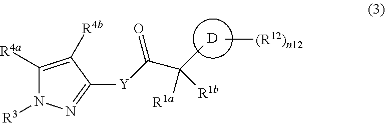

wherein:
D is 5- or 6-membered monocyclic heteroaryl having 1-3 ring heteroatoms independently selected from O, N and S;
each instance of R¹² independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J1}₂, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, or C₁₋₄ haloalkyl,
wherein each of said C₅₋₇ carbocycle and said 5- to 7-membered monocyclic heterocycle is unsubstituted or substituted with halo; and
n12 is 0, 1, 2 or 3.
17. a. The chemical entity of embodiment 5 or 6, which is a chemical entity of Formula (II.A):

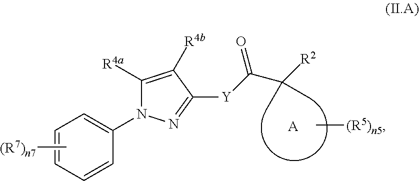

each instance of R⁷ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR³, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl; and
n7 is 0, 1, 2 or 3; or

b. The chemical entity of embodiment 5 or 6, which is a chemical entity of Formula (II.A):

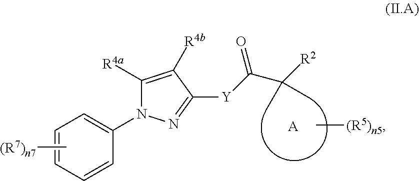

each instance of R⁷ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂—C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl; and
n7 is 0, 1, 2 or 3.
18. a. The chemical entity of embodiment 5 or 6, which is a chemical entity of Formula (II.B):

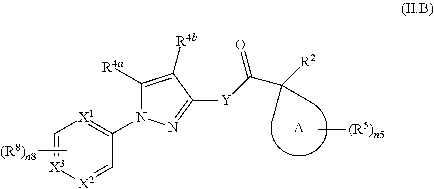

wherein:
one of X¹, X² and X³ is N, and the other two are carbon atoms;
each instance of R⁸ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, or C₁₋₄ haloalkyl; and
n8 is 0, 1, 2 or 3; or

b. The chemical entity of embodiment 5 or 6, which is a chemical entity of Formula (II.B):

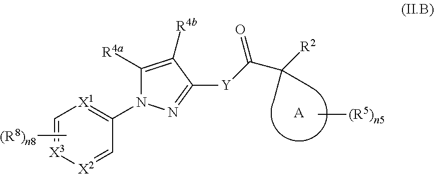

wherein:
one of X¹, X² and X³ is N, and the other two are carbon atoms;
each instance of R⁸ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, or C₁₋₄ haloalkyl; and
n8 is 0, 1, 2 or 3.
19. a. The chemical entity of embodiment 5 or 6, which is a chemical entity of Formula (II.C):

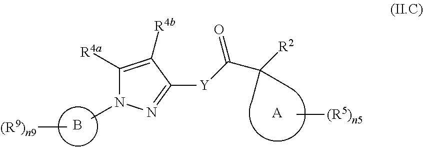

wherein:
B is 5-membered monocyclic heteroaryl having 1-4 ring heteroatoms independently selected from O, N and S, or 6-membered monocyclic heteroaryl having 2 or 3 ring nitrogen atoms;
each instance of R⁹ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, (C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl; and
n9 is 0, 1, 2 or 3; or

b. The chemical entity of embodiment 5 or 6, which is a chemical entity of Formula (II.C):

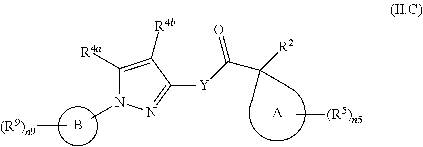

wherein:
B is 5-membered monocyclic heteroaryl having 1-4 ring heteroatoms independently selected from O, N and S, or 6-membered monocyclic heteroaryl having 2 or 3 ring nitrogen atoms;
each instance of R⁹ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl; and
n9 is 0, 1, 2 or 3.
20. a. The chemical entity of embodiment 5 or 6, which is a chemical entity of Formula (II.1):

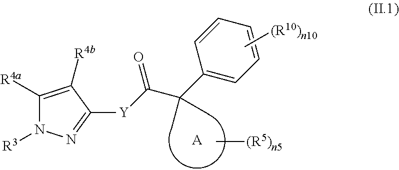

wherein:
each instance of R¹⁰ independently is halo, C₁₋₄ alkyl, C₁₋₄ haloalkyl, —(C(R^{J2a}₂))₀₋₂—OH, —(C(R^{J2a}₂))₀₋₂—OR^{J1}₂, —(C(R^{J2a}₂))₀₋₂—SR^{J2}, —(C(R^{J2a}₂))₀₋₂—NH₂, —(C(R^{J2a}₂))₀₋₂—NHR^{J2}, —(C(R^{J2a}₂))₀₋₂—NR^{J2}₂, —C(O)R^{J2}, or —CN,
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein optionally methylenedioxy constitutes a substituent of said phenyl, wherein the methylene unit of the methylenedioxy is unsubstituted or substituted with halo; and
n10 is 0, 1, 2 or 3; or

b. The chemical entity of embodiment 5 or 6, which is a chemical entity of Formula (II.1):

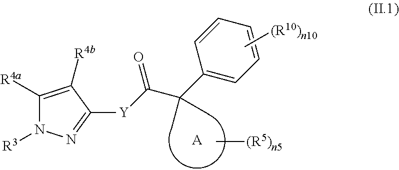

wherein:
each instance of R¹⁰ independently is halo, C₁₋₄ alkyl, C₁₋₄ haloalkyl, —(C(R^{J2a}₂))₀₋₂—OH, —(C(R^{J2a}₂))₀₋₂—OR^{J1}₂, —(C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J2}, or —CN,
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein optionally methylenedioxy constitutes a substituent of said phenyl, wherein the methylene unit of the methylenedioxy is unsubstituted or substituted with halo; and
n10 is 0, 1, 2 or 3.
21. a. The chemical entity of embodiment 7, which is a chemical entity of Formula (III.A):

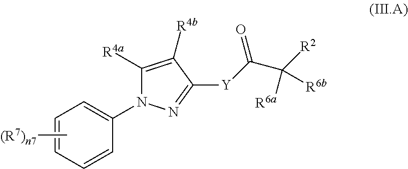

wherein:
each instance of R⁷ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, or C₁₋₄ haloalkyl;
n7 is 0, 1, 2 or 3; or

b. The chemical entity of embodiment 7, which is a chemical entity of Formula (III.A):

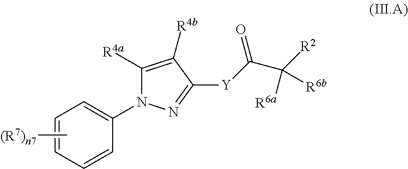

wherein:
each instance of R⁷ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, or C₁₋₄ haloalkyl;
n7 is 0, 1, 2 or 3.
22. a. The chemical entity of embodiment 7, which is a chemical entity of Formula (III.B):

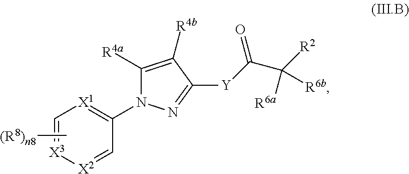

wherein:
one of X¹, X² and X³ is N, and the other two are carbon atoms;
each instance of R⁸ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, or C₁₋₄ haloalkyl; and
n8 is 0, 1, 2 or 3; or

b. The chemical entity of embodiment 7, which is a chemical entity of Formula (III.B):

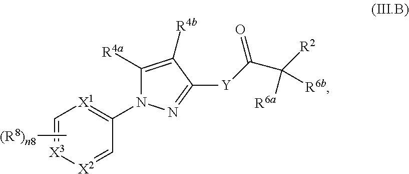

wherein:
one of X¹, X² and X³ is N, and the other two are carbon atoms;
each instance of R⁸ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, or C₁₋₄ haloalkyl; and
n8 is 0, 1, 2 or 3.
23. a. The chemical entity of embodiment 7, which is a chemical entity of Formula (III.C):

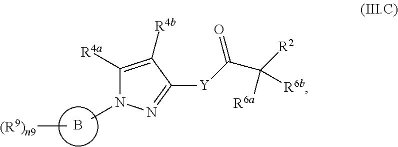

wherein:
B is 5-membered monocyclic heteroaryl having 1-4 ring heteroatoms independently selected from O, N and S, or 6-membered monocyclic heteroaryl having 2 or 3 ring nitrogen atoms;
each instance of R⁹ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, (C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, or C₁₋₄ haloalkyl; and
n9 is 0, 1, 2 or 3; or

b. The chemical entity of embodiment 7, which is a chemical entity of Formula (III.C):

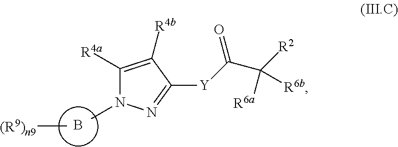

wherein:
B is 5-membered monocyclic heteroaryl having 1-4 ring heteroatoms independently selected from O, N and S, or 6-membered monocyclic heteroaryl having 2 or 3 ring nitrogen atoms;
each instance of R⁹ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, (C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, or C₁₋₄ haloalkyl; and
n9 is 0, 1, 2 or 3.
24. a. The chemical entity of embodiment 7, which is a chemical entity of Formula (III.1):

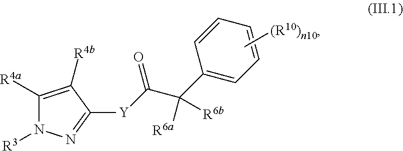

wherein:
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J2a}₂))₀₋₂—SR^{J2}, (C(R^{J2a}₂))₀₋₂—NH₂, —(C(R^{J2a}₂))₀₋₂—NHR^{J2}, (C(R^{J2a}₂))₀₋₂—NR^{J2}₂, —C(O)R^{J1}₂, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, or C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3; or

b. The chemical entity of embodiment 7, which is a chemical entity of Formula (III.1):

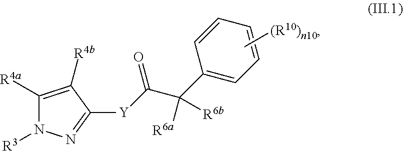

wherein:
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J2}, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, or C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3.
25. a. The chemical entity of embodiment 8, which is a chemical entity of Formula (A.1):

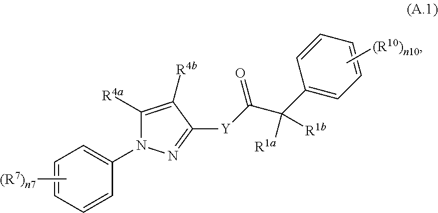

wherein:
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J2a}₂))₀₋₂—SR^{J2}, (C(R^{J2a}₂))₀₋₂—NH₂, —(C(R^{J2a}₂))₀₋₂—NHR^{J2}, (C(R^{J2a}₂))₀₋₂—NR^{J2}₂, —C(O)R^{J2}, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3; or

b. The chemical entity of embodiment 8, which is a chemical entity of Formula (A.1):

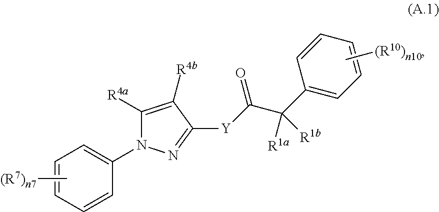

wherein:
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J2}, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3.
26. a. The chemical entity of any one of embodiments 9-12, which is a chemical entity of Formula (B.1):

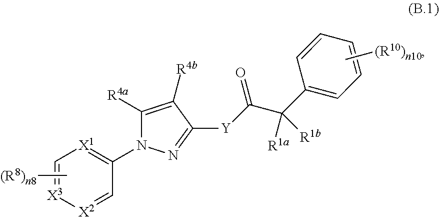

wherein:
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J2a}₂))₀₋₂—SR^{J2} (C(R^{J2a}₂))₀₋₂—NH₂, —(C(R^{J2a}₂))₀₋₂—NHR^{J2}, (C(R^{J2a}₂))₀₋₂—NR^{J2}₂, —C(O)R^{J2}, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3; or

b. The chemical entity of any one of embodiments 9-12, which is a chemical entity of Formula (B.1):

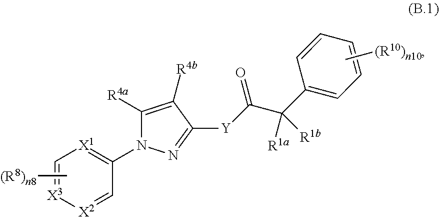

wherein:
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J2}, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3.
27. a. The chemical entity of embodiment 14 or 15, which is a chemical entity of Formula (C.1):

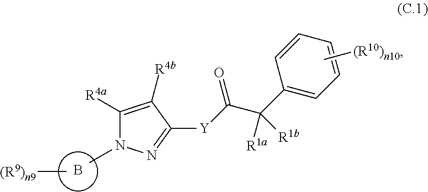

wherein:
B is 5-membered monocyclic heteroaryl having 1-4 ring heteroatoms independently selected from O, N and S, or 6-membered monocyclic heteroaryl having 2 or 3 ring nitrogen atoms;
each instance of R⁹ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl;
n9 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J2a}₂))₀₋₂—SR^{J2}, —(C(R^{J2a}₂))₀₋₂—NH₂, —(C(R^{J2a}₂))₀₋₂—NHR^{J2}, (C(R^{J2a}₂))_{0- 2}—NR^{J2}₂, —C(O)R^{J1}₂, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3; or

b. The chemical entity of embodiment 14 or 15, which is a chemical entity of Formula (C.1):

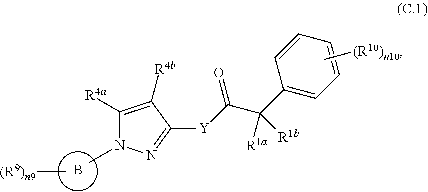

wherein:
B is 5-membered monocyclic heteroaryl having 1-4 ring heteroatoms independently selected from O, N and S, or 6-membered monocyclic heteroaryl having 2 or 3 ring nitrogen atoms;
each instance of R⁹ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, (C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl;
n9 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J1}₂, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3.
28. a. The chemical entity of embodiment 17 or 20, which is a chemical entity of Formula (II.A.1):

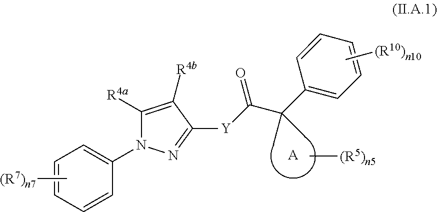

wherein:
each instance of R⁷ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN;
wherein each instance of R^{J3} is independently C₁₋₃ alkyl or C₁₋₄ haloalkyl;
wherein each instance of R^{J3a} is independently H, C₁₋₃ alkyl, C₁₋₄ haloalkyl;
n7 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J2a}₂))₀₋₂—SR^{J2}, (C(R^{J2a}₂))₀₋₂—NH₂, —(C(R^{J2a}₂))₀₋₂—NHR^{J2}, (C(R^{J2a}₂))₀₋₂—NR^{J2}₂, —C(O)R^{J2}, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl;
wherein each instance of R^{J2a} is independently H, C₁₋₃ alkyl, C₁₋₄ haloalkyl;
wherein each of said C₅₋₇ cycloalkyl and 5- to 7-membered monocyclic heterocycle is unsubstituted or substituted with halo; and
n10 is 0, 1, 2 or 3; or

b. The chemical entity of embodiment 17 or 20, which is a chemical entity of Formula (II.A.1):

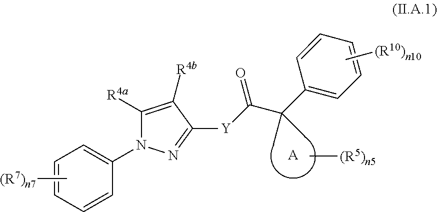

wherein:
each instance of R⁷ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl;
n7 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J1}₂, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3.
29. a. The chemical entity of embodiment 18 or 20, which is a chemical entity of Formula (II.B.1):

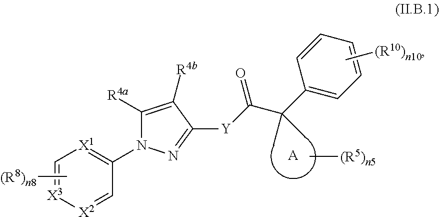

wherein:
one of X¹, X² and X³ is N, and the other two are carbon atoms;
each instance of R⁸ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl;
n8 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J2a}₂))₀₋₂—SR^{J2}, (C(R^{J2a}₂))₀₋₂—NH₂, —(C(R^{J2a}₂))₀₋₂—NHR^{J2}, (C(R^{J2a}₂))₀₋₂—NR^{J2}₂, —C(O)R^{J1}₂, and —CN,
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein optionally methylenedioxy constitutes a substituent of said phenyl, wherein the methylene unit of the methylenedioxy is unsubstituted or substituted with halo; and
n10 is 0, 1, 2 or 3; or

b. The chemical entity of embodiment 18 or 20, which is a chemical entity of Formula (II.B.1):

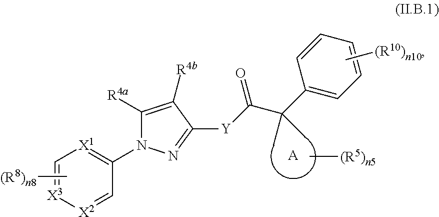

wherein:
one of X¹, X² and X³ is N, and the other two are carbon atoms;
each instance of R⁸ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl;
n8 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J1}₂, and —CN,
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein optionally methylenedioxy constitutes a substituent of said phenyl, wherein the methylene unit of the methylenedioxy is unsubstituted or substituted with halo; and
n10 is 0, 1, 2 or 3.
30. a. The chemical entity of embodiment 19 or 20, which is a chemical entity of Formula (II.C.1):

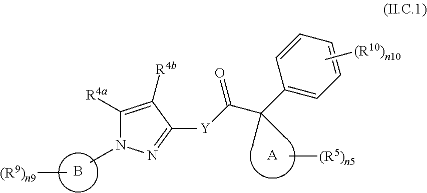

B is 5-membered monocyclic heteroaryl having 1-4 ring heteroatoms independently selected from O, N and S, or 6-membered monocyclic heteroaryl having 2 or 3 ring nitrogen atoms;
each instance of R⁹ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, (C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR³², —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl;
n9 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J2a}₂))₀₋₂—SR^{J2}, (C(R^{J2a}₂))₀₋₂—NH₂, —(C(R^{J2a}₂))₀₋₂—NHR^{J2}, (C(R^{J2a}₂))₀₋₂—NR^{J2}₂, —C(O)R^{J1}₂, and —CN,
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein optionally methylenedioxy constitutes a substituent of said phenyl, wherein the methylene unit of the methylenedioxy is unsubstituted or substituted with halo; and
n10 is 0, 1, 2 or 3; or

b. The chemical entity of embodiment 19 or 20, which is a chemical entity of Formula (II.C.1):

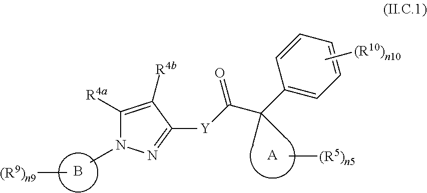

B is 5-membered monocyclic heteroaryl having 1-4 ring heteroatoms independently selected from O, N and S, or 6-membered monocyclic heteroaryl having 2 or 3 ring nitrogen atoms;
each instance of R⁹ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, (C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl;
n9 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J1}₂, and —CN,
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein optionally methylenedioxy constitutes a substituent of said phenyl, wherein the methylene unit of the methylenedioxy is unsubstituted or substituted with halo; and
n10 is 0, 1, 2 or 3.
31. a. The chemical entity of embodiment 21 or 24, which is a chemical entity of Formula (III.A.1):

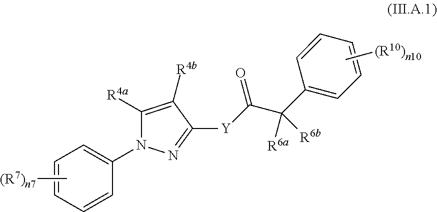

wherein:
each instance of R⁷ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂—C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl;
n7 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J2a}₂))₀₋₂—SR^{J2}, (C(R^{J2a}₂))₀₋₂—NH₂, —(C(R^{J2a}₂))₀₋₂—NHR^{J2}, (C(R^{J2a}₂))₀₋₂—NR^{J2}₂, —C(O)R^{J1}₂, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3; or

b. The chemical entity of embodiment 21 or 24, which is a chemical entity of Formula (III.A.1):

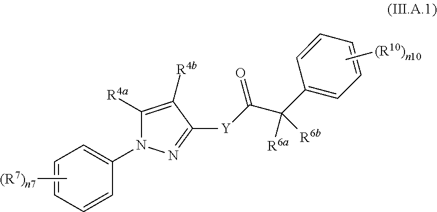

wherein:
each instance of R⁷ independently is selected from halo, C₁₋₄ alkyl, C₁₋₄ haloalkyl, —(C(R^{J3a}₂))₀₋₂—OH, —(C(R^{J3a}₂))₀₋₂—OR³, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl;
n7 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J1}₂, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3.
32. The chemical entity of embodiment 31, which is a chemical entity of Formula (III.A.1a):

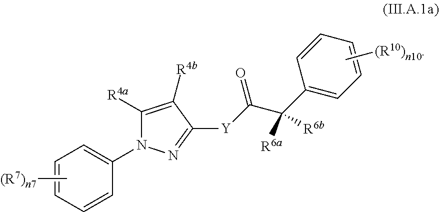

33. The chemical entity of embodiment 31, which is a chemical entity of Formula (III.A.1b):

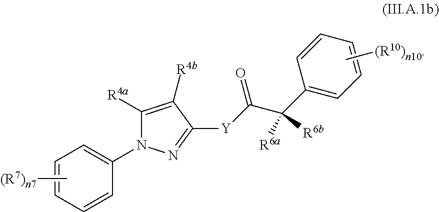

34. a. The chemical entity of embodiment 21, which is a chemical entity of Formula (III.A.3):

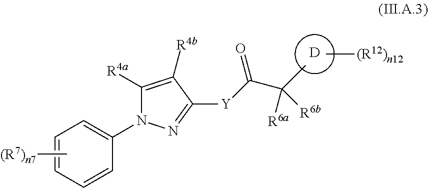

wherein:
D is 5- or 6-membered monocyclic heteroaryl having 1-3 ring heteroatoms independently selected from O, N and S;
each instance of R¹² independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J2a}₂))₀₋₂—SR^{J2}, (C(R^{J2a}₂))₀₋₂—NH₂, —(C(R^{J2a}₂))₀₋₂—NHR^{J2}, (C(R^{J2a}₂))₀₋₂—NR^{J2}₂, —C(O)R^{J1}₂, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, or C₁₋₄ haloalkyl,
wherein each of said C₅₋₇ carbocycle and said 5- to 7-membered monocyclic heterocycle is unsubstituted or substituted with halo; and
n12 is 0, 1, 2 or 3; or

b. The chemical entity of embodiment 21, which is a chemical entity of Formula (III.A.3):

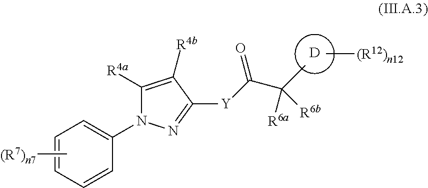

wherein:
D is 5- or 6-membered monocyclic heteroaryl having 1-3 ring heteroatoms independently selected from O, N and S;
each instance of R¹² independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J1}₂, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, or C₁₋₄ haloalkyl,
wherein each of said C₅₋₇ carbocycle and said 5- to 7-membered monocyclic heterocycle is unsubstituted or substituted with halo; and
n12 is 0, 1, 2 or 3.
35. a. The chemical entity of embodiment 22 or 24, which is a chemical entity of Formula (III.B.1):

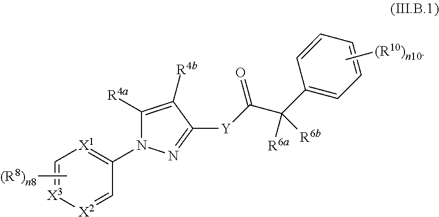

wherein:
one of X¹, X² and X³ is N, and the other two are carbon atoms;
each instance of R⁸ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl;
n8 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J2a}₂))₀₋₂—SR^{J2}, (C(R^{J2a}₂))₀₋₂—NH₂, —(C(R^{J2a}₂))₀₋₂—NHR^{J2}, (C(R^{J2a}₂))_{0- 2}—NR^{J2}₂, —C(O)R^{J1}₂, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3; or

b. The chemical entity of embodiment 22 or 24, which is a chemical entity of Formula (III.B.1):

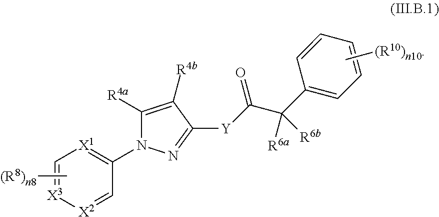

wherein:
one of X¹, X² and X³ is N, and the other two are carbon atoms;
each instance of R⁸ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl;
n8 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J1}₂, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3.
36. a. The chemical entity of embodiment 23 or 24, which is a chemical entity of Formula (III.C.1):

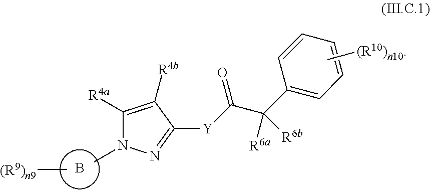

wherein:
B is 5-membered monocyclic heteroaryl having 1-4 ring heteroatoms independently selected from O, N and S, or 6-membered monocyclic heteroaryl having 2 or 3 ring nitrogen atoms;
each instance of R⁹ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, (C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl;
n9 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J2a}₂))₀₋₂—SR^{J2}, (C(R^{J2a}₂))₀₋₂—NH₂, —(C(R^{J2a}₂))₀₋₂—NHR^{J2}, (C(R^{J2a}₂))₀₋₂—NR^{J2}₂, —C(O)R^{J1}₂, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3; or

b. The chemical entity of embodiment 23 or 24, which is a chemical entity of Formula (III.C.1):

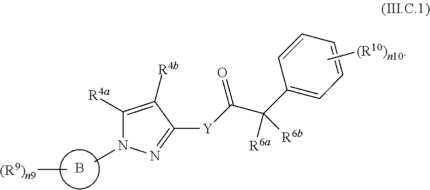

wherein:
B is 5-membered monocyclic heteroaryl having 1-4 ring heteroatoms independently selected from O, N and S, or 6-membered monocyclic heteroaryl having 2 or 3 ring nitrogen atoms;
each instance of R⁹ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl;
n9 is 0, 1, 2 or 3;
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, (C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J1}₂, and —CN, or two adjacent R¹⁰ forms methylenedioxy, wherein the methylene unit of the methylenedioxy is unsubstituted or substituted with halo;
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein each of said C₅₋₇ cycloalkyl and 5- to 7-membered monocyclic heterocycle is unsubstituted or substituted with halo; and
n10 is 0, 1, 2 or 3.
37. The chemical entity of any one of embodiments 9, 18, 22, 26, 29 and 35, wherein X¹ is N, and X² and X³ are CH.
38. The chemical entity of any one of embodiments 9, 18, 22, 26, 29 and 35, wherein X² is N, and X¹ and X³ are CH.
39. The chemical entity of any one of embodiments 9, 18, 22, 26, 29 and 35, wherein X³ is N, and X¹ and X² are CH.
40. The chemical entity of any one of embodiments 5, 17-20 and 28-30, wherein A is cyclopropane, cyclobutane, cyclopentane, cyclohexane, azetidine, oxetane, pyrrolidine, tetrahydrofuran, tetrahydrothiophene, piperidine, tetrahydropyran or tetrahydrothiopyran, wherein the heteroatom of the foregoing applicable rings are not bonded to the carbon to which A is attached, and wherein each of the foregoing rings is unsubstituted or substituted with 1-2 instances of R⁵, wherein each instance of R⁵ independently is selected from halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, —(C(R^{J1a}₂))₀₋₂—NH₂, —(C(R^{J1a}₂))₀₋₂—NHR^{J1}, and —(C(R^{J1a}₂))₀₋₂—NR^{J1}₂, or two geminal R⁵, together with the carbon atom to which they are attached, form a C₄₋₆ cycloalkyl or 4- to 6-membered monocyclic heterocycle containing 1-2 heteroatoms selected from O, N, and S.
41. The chemical entity of embodiment 40, wherein A is cyclopropane, cyclobutane, cyclopentane, cyclohexane, tetrahydrofuran, tetrahydrothiophene, piperidine or tetrahydropyran.
42. The chemical entity of embodiment 40, wherein A is cyclopropane, cyclobutane, cyclopentane, cyclohexane, pyrrolidine, oxetane or tetrahydropyran.
43. The chemical entity of embodiment 40, wherein A is pyrrolidine, oxetane or tetrahydropyran.
44. The chemical entity of embodiment 40, wherein A is cyclopropane, cyclobutane, cyclopentane, cyclohexane, oxetane or tetrahydropyran.
45. The chemical entity of embodiment 40, wherein A is oxetane, tetrahydrofuran, or tetrahydropyran.
46. The chemical entity of embodiment 40, wherein A is cyclopropane, cyclobutane, cyclopentane or cyclohexane.
47. The chemical entity of embodiment 40, wherein A is cyclopropane or cyclobutane.
48. The chemical entity of embodiment 40, wherein A is cyclopropane.
49. The chemical entity of any one of embodiments 40 to 48, wherein each instance of R⁵ independently is C₁₋₄ alkyl or halo, or two geminal R⁵, together with the carbon atom to which they are attached, form a C₄₋₆ carbocycle.
50. The chemical entity of embodiment 49, wherein two geminal R⁵, together with the carbon atom to which they are attached, form cyclobutane or cyclopentane.
51. The chemical entity of embodiment 49, wherein each instance of R⁵ independently is C₁₋₄ alkyl.
52. The chemical entity of embodiment 51, wherein each instance of R⁵ is Me.
53. The chemical entity of embodiment 49, wherein each instance of R⁵ independently is halo.
54. The chemical entity of embodiment 53, wherein each instance of R⁵ independently is —F or —Cl.
55. The chemical entity of any one of embodiments 40 to 54, wherein n5 is 0, 1 or 2.
56. The chemical entity of any one of embodiments 40 to 54, wherein n5 is 0.
57. The chemical entity of any one of embodiments 40 to 54, wherein n5 is 1.
58. The chemical entity of any one of embodiments 40 to 54, wherein n5 is 2.
59. The chemical entity of any one of embodiments 40 to 54, wherein n5 is 2 and (R⁵)ₙ₅ is geminal di-(C₁₋₄ alkyl) or geminal di-halo.
60. The chemical entity of any one of embodiments 40 to 54, wherein n5 is 2 and (R⁵)ₙ₅ is geminal dimethyl.
61. The chemical entity of any one of embodiments 40 to 54, wherein n5 is 2 and (R⁵)ₙ₅ is geminal difluoro or geminal dichloro.
62. The chemical entity of embodiment 61, wherein n5 is 2 and (R⁵)ₙ₅ is geminal difluoro.
63. The chemical entity of any one of embodiments 40 to 54, wherein n5 is 2 and two geminal R⁵, together with the carbon atom to which they are attached, form cyclobutane or cyclopentane.
64. The chemical entity of any one of embodiments 1, 2, 17 to 20, 28 to 30 and 40 to 46, wherein A is cyclopropane, cyclobutane or cyclopentane; n5 is 2; and (R⁵)ₙ₅ is geminal dimethyl, geminal difluoro or geminal dichloro.
65. The chemical entity of any one of embodiments 1, 2, 17 to 20, 28 to 30 and 40 to 46, wherein A is cyclopropane, cyclobutane or cyclopentane; n5 is 2; and (R⁵)ₙ₅ is geminal difluoro or geminal dichloro.
66. The chemical entity of any one of embodiments 1, 2, 17 to 20, 28 to 30 and 40 to 46, wherein A is cyclopropane, cyclobutane or cyclopentane, and n5 is 0.
67. The chemical entity of any one of embodiments 1, 2, 17 to 20, 28 to 30 and 40 to 46, wherein A is cyclopropane or cyclobutane, and n5 is 0.
68. The chemical entity of any one of embodiments 1, 2, 17 to 20, 28 to 30 and 40 to 46, wherein A is cyclopropane and n5 is 0.
69. The chemical entity of any one of embodiments 15, 20, 24 to 33, 35 and 36, wherein each instance of R¹⁰ independently is —F, —Cl, Me, Et, Pr, Bu, iPr, iBu, —OH, —OMe, —OEt, —OPr, —OiPr, NH₂, —NHMe, —NHEt, —NHiPr, —CF₃, —CHF₂ or —CN.
70. The chemical entity of any one of embodiments 15, 20, 24 to 33, 35 and 36, wherein each instance of R¹⁰ independently is Me, Et, Pr, Bu, ⁱPr, ⁱBu, sec-Bu, —F, —Cl, —CF₃, —CHF₂, —OCF₃, —OH, —OMe, —OEt, —OPr, —O-ⁱPr, —NH₂, —NHMe, —NHPr, —SO₂NH₂, —SO₂NHMe, or —CN.
71. The chemical entity of any one of embodiments 15, 20, 24 to 33, 35 and 36, wherein each instance of R¹⁰ independently is Me, ⁱPr, ⁱBu, —F, —Cl, —CF₃, —OCF₃, —OH, —OMe, or —OEt.
72. The chemical entity of embodiment 69, wherein each instance of R¹⁰ independently is —F, —Cl, Me, Et, Pr, Bu, iPr, iBu, —OH, —OMe, —OEt, —OPr, —OiPr, —NH₂, —NHMe, —CF₃ or —CN.
73. The chemical entity of embodiment 69, wherein each instance of R¹⁰ independently is —F, —Cl, Me, —OMe, —OEt, —CN or —CF₃.
74. The chemical entity of embodiment 69, wherein each instance of R¹⁰ independently is —F, —Cl, Me, —OMe, —OEt or —CN.
75. The chemical entity of embodiment 69, wherein each instance of R¹⁰ independently is —F, —Cl, Me, —CF₃ or —CN.
76. The chemical entity of embodiment 69, wherein each instance of R¹⁰ independently is —F, —Cl or Me.
77. The chemical entity of embodiment 69, wherein each instance of R¹⁰ independently is —F, —Cl or —CF₃.
78. The chemical entity of embodiment 69, wherein each instance of R¹⁰ independently is —F.
79. The chemical entity of embodiment 69, wherein each instance of R¹⁰ independently is —F, —Cl, Me, Et, —OH, —NH₂ or —CF₃.
80. The chemical entity of embodiment 69, wherein each instance of R¹⁰ independently is —F, —Cl, or Me.
81. The chemical entity of embodiment 69, wherein each instance of R¹⁰ independently is —F, —Cl, Me, Et, ⁱPr, —OH, —OMe, —NH₂, —CF₃ or —CN.
82. The chemical entity of embodiment 69, wherein each instance of R¹⁰ independently is —F, —Cl, Me, —OMe, —OEt or —CN.
83. The chemical entity of embodiment 69, wherein each instance of R¹⁰ is —F.
84. The chemical entity of any one of embodiments 15, 20, 24 to 33, 35, 36 and 69 to 83, wherein n10 is 0, 1, or 2.
85. The chemical entity of any one of embodiments 15, 20, 24 to 33, 35, 36 and 69 to 83, n10 is 0.
86. The chemical entity of any one of embodiments 15, 20, 24 to 33, 35, 36 and 69 to 83, wherein n10 is 0, 1, or 2, and R¹⁰ is —F or Me.
87. The chemical entity of any one of embodiments 15, 20, 24 to 33, 35, 36 and 69 to 83, wherein n10 is 0 or 1, and R¹⁰ is —F, —Cl, Me, Et, —OH, —NH₂ or —CF₃.
88. The chemical entity of any one of embodiments 15, 20, 24 to 33, 35, 36 and 69 to 83, wherein n10 is 0 or 1, and R¹⁰ is —F, —Cl, Me, —CF₃ or —CN.
89. The chemical entity of any one of embodiments 15, 20, 24 to 33, 35, 36 and 69 to 83, n10 is 1 and R¹⁰ is —F.
90. The chemical entity of any one of embodiments 15, 20, 24 to 33, 35, 36 and 69 to 83, wherein n10 is 0 or 1, and R¹⁰ is —F, —Cl, Me, —OMe, —OEt or —CN.
91. The chemical entity of any one of embodiments 15, 20, 24 to 33, 35, 36 and 69 to 83, wherein n10 is 1 and R¹⁰ is —F.
92. The chemical entity of any one of embodiments 15, 20, 24 to 33, 35, 36 and 69 to 83, wherein n10 is 0 or 1, and R¹⁰ is —F, —Cl, Me, —CF₃ or —CN.
93. The chemical entity of any one of embodiments 15, 20, 24 to 33, 35, 36 and 69 to 83, wherein n10 is 1 and R¹⁰ is —F.
94. The chemical entity of any one of embodiments 7, 21 to 24, 31 to 36 and 69 to 83, wherein R^{6a} is Me, Et, Pr, Bu, ⁱPr, ⁱBu, sec-Bu, cyclopropyl, cyclobutyl, cyclopentyl, cyclohexyl, —CF₃, or OH, and R^{6b} is —H.
95. The chemical entity of any one of embodiments 7, 21 to 24, 31 to 36 and 69 to 83, wherein each of R^{6a} and R^{6b} independently is —H, Me, Et or Pr.
96. The chemical entity of any one of embodiments 7, 21 to 24, 31 to 36 and 69 to 83, wherein each of R^{6a} and R^{6b} independently is —H, Me, Et, Pr, cyclopropyl or cyclopentyl.
97. The chemical entity of any one of embodiments 7, 21 to 24, 31 to 36 and 69 to 83, wherein R^{6a} is Me, Et, Pr, ⁱPr, cyclopropyl or cyclopentyl.
98. The chemical entity of any one of embodiments 7, 21 to 24, 31 to 36 and 69 to 83, wherein R^{6a} is Me, Et, iPr or —CF₃, and R^{6b} is Me, Et, iPr, cyclopropyl, cyclobutyl or cyclopentyl.
99. The chemical entity of any one of embodiments 7, 21 to 24, 31 to 36 and 69 to 83, wherein R^{6a} is Me, Et, Pr, or —CF₃, and R^{6b} is Me, Et, Pr, cyclopropyl, cyclobutyl or cyclopentyl.
100. The chemical entity of any one of embodiments 7, 21 to 24, 31 to 36 and 69 to 83, wherein R^{6a} is Me, Et, cyclopropyl, cyclobutyl, or —CF₃, and R^{6b} is —H.
101. The chemical entity of any one of embodiments any one of embodiments 7, 21 to 24, 31 to 36 and 69 to 83, wherein each of R^{6a} and R^{6b} is —H.
102. The chemical entity of any one of the preceding embodiments, wherein each of R^{4a} and R^{4b} independently is —H, F, Me, Et, Pr, Bu, iPr, or iBu.
103. The chemical entity of any one of the preceding embodiments, wherein R^{4a} is H and R^{4b} is Me.
104. The chemical entity of any one of the preceding embodiments, wherein R^{4a} is Me and R^{4b} is H.
105. The chemical entity of any one of the preceding embodiments, wherein R^{4a} is —H.
106. The chemical entity of any one of the preceding embodiments, wherein R^{4b} is —H.
107. The chemical entity of any one of the preceding embodiments, wherein each of R^{4a} and R^{4b} is —H.
108. The chemical entity of any one of embodiments 8, 17, 21, 25, 28 and 31 to 34, wherein R⁷ is —F, —Cl, Me, Et, Pr, Bu, iPr, iBu, —OH, —OMe, —OEt, —OPr, —OiPr, NH₂, —NHMe, NHEt, NHⁱPr, —CF₃, —CHF₂ or —CN.
109. The chemical entity of any one of embodiments 8, 17, 21, 25, 28 and 31 to 34, wherein R⁷ is —F, —Cl or —CF₃.
110. The chemical entity of any one of embodiments 8, 17, 21, 25, 28 and 31 to 34, wherein each instance of R⁷ independently is Me, Et, Pr, Bu, ⁱPr, ⁱBu, sec-Bu, —F, —Cl, —CF₃, —CHF₂, —OCF₃, —OH, —OMe, —OEt, —OPr, —O-iPr, —NH₂, —NHMe, —NHPr, or —CN.
111. The chemical entity of any one of embodiments 8, 17, 21, 25, 28 and 31 to 34, wherein each instance of R⁷ independently is —F, —Cl, —CF₃ or —OH.
112. The chemical entity of any one of embodiments 8, 17, 21, 25, 28 and 31 to 34, wherein each instance of R⁷ independently is —F or —Cl.
113. The chemical entity of any one of embodiments 8, 17, 21, 25, 28 and 31 to 34, wherein R⁷ is —F.
114. The chemical entity of any one of embodiments 8, 17, 21, 25, 28 and 31 to 34, wherein n7 is 0, 1, or 2.
115. The chemical entity of any one of embodiments 8, 17, 21, 25, 28 and 31 to 34, wherein n7 is 0 or 1, and R⁷ is —F, —Cl or —CF₃.
116. The chemical entity of any one of embodiments 8, 17, 21, 25, 28 and 31 to 34, wherein n7 is 0, 1 or 2, and each instance of R⁷ independently is —F, —Cl or —CF₃.
117. The chemical entity of any one of embodiments 8, 17, 21, 25, 28 and 31 to 34, wherein n7 is 1 or 2, and each instance of R⁷ independently is —F or —Cl.
118. The chemical entity of any one of embodiments 8, 17, 21, 25, 28 and 31 to 34, wherein n7 is 0 or 1, and each instance of R⁷ independently is —F or —Cl.
119. The chemical entity of any one of embodiments 8, 17, 21, 25, 28 and 31 to 34, wherein n7 is 1 and R⁷ is —F or —Cl.
120. The chemical entity of any one of embodiments 8, 17, 21, 25, 28 and 31 to 34, wherein n7 is 1 and R⁷ is —F.
121. The chemical entity of any one of embodiments 9, 18, 22, 26, 29 and 35, wherein each instance of R⁸ independently is halo, C₁₋₄ alkyl, C₁₋₄ haloalkyl, —OH, —OMe or —OEt.
122. The chemical entity of any one of embodiments 9, 18, 22, 26, 29 and 35, wherein each instance of R⁸ independently is —F, —Cl, Me, Et, Pr, Bu, iPr, iBu, —OH, —OMe, —OEt, —OPr, —OiPr, —NH₂, —NHMe, —NHEt, —NHiPr, —CF₃, —CHF₂ and —CN.
123. The chemical entity of any one of embodiments 9, 18, 22, 26, 29 and 35, wherein each instance of R⁸ independently is —F, —Cl, Me, Et, —CF₃, —OH, —OMe or —OEt.
124. The chemical entity of any one of embodiments 9, 18, 22, 26, 29 and 35, wherein each instance of R⁸ independently is —F, —Cl, Me, —OMe or —OH.
125. The chemical entity of any one of embodiments 9, 18, 22, 26, 29 and 35, wherein each instance of R⁸ is —F.
126. The chemical entity of any one of embodiments 9, 18, 22, 26, 29 and 35, wherein n8 is 0, 1 or 2.
127. The chemical entity of any one of embodiments 9, 18, 22, 26, 29 and 35, wherein n8 is 0 or 1.
128. The chemical entity of any one of embodiments 9, 18, 22, 26, 29 and 35, wherein n8 is 1.
129. The chemical entity of any one of embodiments 9, 18, 22, 26, 29 and 35, wherein n8 is 0.
130. The chemical entity of any one of embodiments 9, 18, 22, 26, 29 and 35, wherein n8 is 0 or 1, and R⁸ is —F, —Cl, Me, —OMe or —OH.
131. The chemical entity of any one of embodiments 9, 18, 22, 26, 29 and 35, wherein n8 is 1, and R⁸ is —F or —Cl.
132. The chemical entity of any one of embodiments 9, 18, 22, 26, 29 and 35, wherein n8 is 0 or 1, and R⁸ is —F, —Cl, Me, Et, —CF₃, —OH, —OMe or —OEt.
133. The chemical entity of any one of embodiments 9, 18, 22, 26, 29 and 35, wherein n8 is 0, 1 or 2, and each instance of R⁸ independently is —F or —Cl.
134. The chemical entity of any one of the preceding embodiments, wherein Y is —NH— or —N(Me)-.
135. The chemical entity of any one of the preceding embodiments, wherein Y is —NH—.
136. The chemical entity of any one of embodiments 14, 19, 23, 27, 30 and 36, wherein B is pyrazolyl, thiazolyl, isothiazolyl, pyrimidinyl, pyrazinyl or pyridazinyl.
137. The chemical entity of any one of embodiments 14, 19, 23, 27, 30 and 36, wherein B is pyrimidinyl, thiazolyl, pyrazinyl or pyridazinyl.
138. The chemical entity of any one of embodiments 14, 19, 23, 27, 30 and 36, wherein B is thienyl, thiazolyl, pyrimidinyl, pyrazolyl, pyrazinyl or pyridyl.
139. The chemical entity of any one of embodiments 14, 19, 23, 27, 30 and 36, wherein B is thiazolyl or pyrimidinyl.
140. The chemical entity of any one of embodiments 14, 19, 23, 27, 30 and 36, wherein each instance of R⁹ independently is —F, —Cl, Me, Et, —OH, —NH₂ or —CF₃
141. The chemical entity of any one of embodiments 14, 19, 23, 27, 30 and 36, wherein each instance of R⁹ independently is —F, —Cl, or Me.
142. The chemical entity of any one of embodiments 14, 19, 23, 27, 30 and 36, wherein each instance of R⁹ is Me.
143. The chemical entity of any one of embodiments 14, 19, 23, 27, 30 and 36, wherein n9 is 0, 1 or 2.
144. The chemical entity of any one of embodiments 14, 19, 23, 27, 30 and 36, wherein n9 is 0.
145. The chemical entity of any one of embodiments 14, 19, 23, 27, 30 and 36, wherein n9 is 0, 1 or 2, and each instance of R⁹ independently is —F, —Cl, Me, Et, or —CF₃.
146. The chemical entity of any one of embodiments 14, 19, 23, 27, 30 and 36, wherein n9 is 0 or 1, and R⁹ is Me or -D.
147. The chemical entity of any one of embodiments 14, 19, 23, 27, 30 and 36, wherein n9 is 1 or 2, and each instance of R⁹ independently is —F or Me.
148. The chemical entity of any one of embodiments 14, 19, 23, 27, 30 and 36, wherein n9 is 1 and R⁹ is Me.
149. The chemical entity of any one of embodiments 14, 19, 23, 27, 30 and 36, wherein B is pyrazolyl, thiazolyl, pyrazinyl or pyridazinyl; n9 is 0 or 1, and R⁹ is Me.
150. The chemical entity of any one of embodiments 14, 19, 23, 27, 30 and 36, wherein B is pyrimidinyl or thiazolyl, and n9 is 0.
151. The chemical entity of any one of embodiments 16 or 34, wherein D is thienyl, thiazolyl, pyrimidinyl, pyrazolyl, pyrazinyl or pyridyl.
152. The chemical entity of embodiment 16 or 34, wherein D is pyrimidinyl or pyridyl.
153. The chemical entity of embodiment 16 or 34, wherein n12 is 0 or 1.
154. The chemical entity of embodiment 16 or 34, wherein n12 is 0 or 1, and R¹² is Me.
155. The chemical entity of embodiment 16 or 34, wherein D is thienyl, thiazolyl, pyrimidinyl, pyrazolyl, pyrazinyl, or pyridyl; n12 is 0 or 1; and R¹² is Me.
156. A chemical entity selected from the list of free compounds in Table 1 and pharmaceutically acceptable salts thereof.
157. The chemical entity according to embodiment 1, which is the free compound

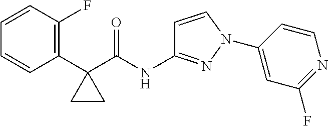 [SMILES: O=C(Nc1ccn(-c2ccnc(F)c2)n1)C1(c2ccccc2F)CC1]

1-(2-fluorophenyl)-N-[1-(2-fluoro-4-pyridyl)pyrazol-3-yl]cyclopropanecarboxamide (Compound 87) or which is a pharmaceutically acceptable salt thereof.
158. The chemical entity according to embodiment 1, which is the free compound

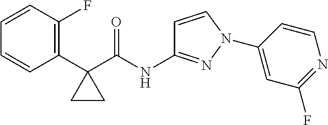 [SMILES: O=C(Nc1ccn(-c2ccnc(F)c2)n1)C1(c2ccccc2F)CC1]

1-(2-fluorophenyl)-N-[1-(2-fluoro-4-pyridyl)pyrazol-3-yl]cyclopropanecarboxamide (Compound 87).
159. The chemical entity according to embodiment 1, which is the free compound

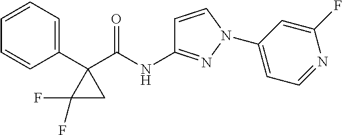 [SMILES: O=C(Nc1ccn(-c2ccnc(F)c2)n1)C1(c2ccccc2)CC1(F)F]

2,2-difluoro-N-(1-(2-fluoropyridin-4-yl)-1H-pyrazol-3-yl)-1-phenylcyclopropane-1-carboxamide (Compound 169) or which is a pharmaceutically acceptable salt thereof.
160. The chemical entity according to embodiment 159, which is the free compound selected from

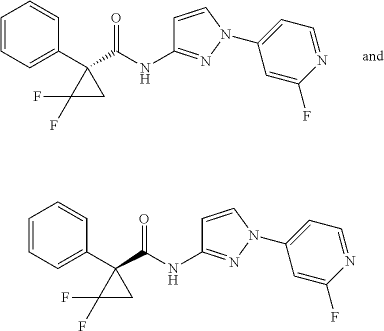 [SMILES: O=C(Nc1ccn(-c2ccnc(F)c2)n1)[C@]1(c2ccccc2)CC1(F)F ; O=C(Nc1ccn(-c2ccnc(F)c2)n1)[C@@]1(c2ccccc2)CC1(F)F]

or which is a pharmaceutically acceptable salt thereof.
161. The chemical entity according to embodiment 1, which is the free compound

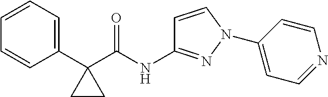 [SMILES: O=C(Nc1ccn(-c2ccncc2)n1)C1(c2ccccc2)CC1]

1-phenyl-N-[1-(4-pyridyl)pyrazol-3-yl]cyclopropanecarboxamide (Compound 100) or which is a pharmaceutically acceptable salt thereof.
162. The chemical entity according to embodiment 1, which is the free compound

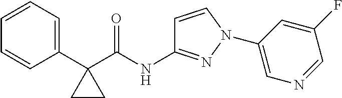 [SMILES: O=C(Nc1ccn(-c2cncc(F)c2)n1)C1(c2ccccc2)CC1]

N-[1-(5-fluoro-3-pyridyl)pyrazol-3-yl]-1-phenyl-cyclopropanecarboxamide (Compound 201) or which is a pharmaceutically acceptable salt thereof.
163. The chemical entity according to embodiment 1, which is the free compound

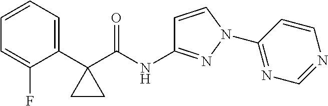 [SMILES: O=C(Nc1ccn(-c2ccncn2)n1)C1(c2ccccc2F)CC1]

1-(2-fluorophenyl)-N-(1-pyrimidin-4-ylpyrazol-3-yl)cyclopropanecarboxamide (Compound 206) or which is a pharmaceutically acceptable salt thereof.
164. The chemical entity according to embodiment 1, which is the free compound

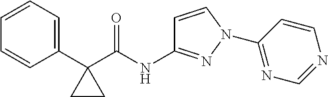 [SMILES: O=C(Nc1ccn(-c2ccncn2)n1)C1(c2ccccc2)CC1]

1-phenyl-N-(1-pyrimidin-4-ylpyrazol-3-yl)cyclopropanecarboxamide (Compound 207) or which is a pharmaceutically acceptable salt thereof.
165. The chemical entity according to embodiment 1, which is the free compound

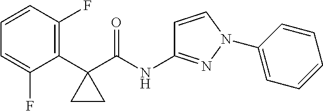 [SMILES: O=C(Nc1ccn(-c2ccccc2)n1)C1(c2c(F)cccc2F)CC1]

1-(2,6-difluorophenyl)-N-(1-phenylpyrazol-3-yl)cyclopropanecarboxamide (Compound 267) or which is a pharmaceutically acceptable salt thereof.
166. The chemical entity according to embodiment 1, which is the free compound

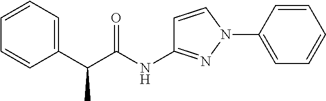 [SMILES: C[C@H](C(=O)Nc1ccn(-c2ccccc2)n1)c1ccccc1]

(2S)-2-phenyl-N-(1-phenylpyrazol-3-yl)propanamide (Compound 20) or which is a pharmaceutically acceptable salt thereof.
167. The chemical entity according to embodiment 1, which is the free compound

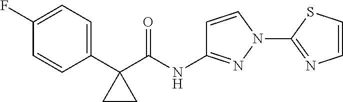 [SMILES: O=C(Nc1ccn(-c2nccs2)n1)C1(c2ccc(F)cc2)CC1]

1-(2-fluorophenyl)-N-(1-thiazol-2-ylpyrazol-3-yl)cyclopropanecarboxamide (Compound 92) or which is a pharmaceutically acceptable salt thereof.
168. The chemical entity according to embodiment 1, which is the a compound selected from

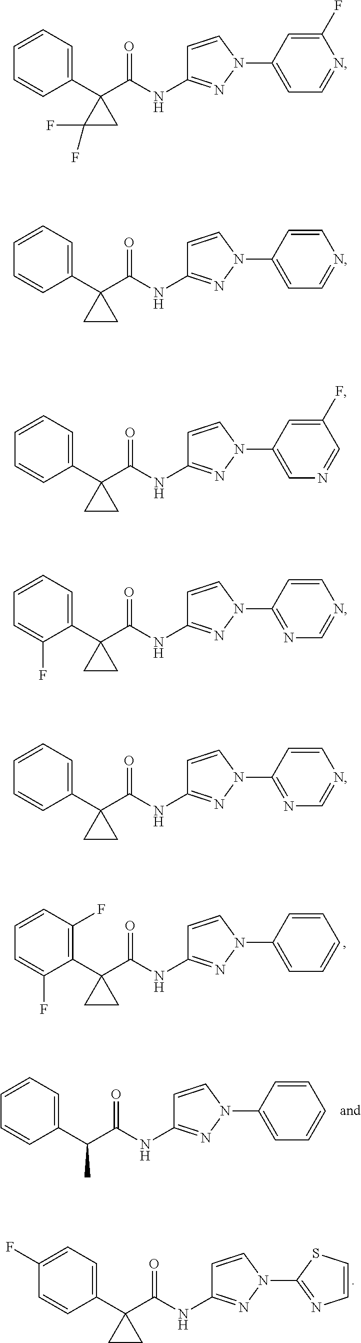

169. A pharmaceutical composition comprising a chemical entity of any one of embodiments 1-168 and a pharmaceutically acceptable carrier, adjuvant, or excipient.
170. a. A method of treating a disease, disorder or condition in a subject comprising administering to the subject an effective amount of a chemical entity, which is a free compound of Formula (I) or a pharmaceutically acceptable salt thereof, wherein Formula (I) has the structure,

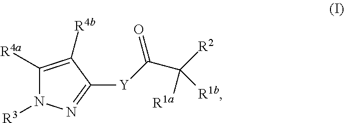

each of R^{1a} and R^{1b} independently is H, —C₁₋₄ alkyl, C₁₋₄ haloalkyl, —(C(R^{J1a}₂))₁₋₂—OH, —(C(R^{J1a}₂))₁₋₂—OR^{J1}, —(C(R^{J1a}₂))₁₋₂—SR^{J1}, —(C(R^{J1a}₂))₁₋₂—NH₂, —(C(R^{J1a}₂))₁₋₂—NHR^{J1}, —(C(R^{J1a}₂))₁₋₂—NR^{J1}₂, C₃₋₆ cycloalkyl or a 3- to 6-membered monocyclic heterocycle containing 1 ring heteroatom selected from O, N, and S,
wherein the 3- to 6-membered monocyclic heterocycle does not contain a heteroatom bonded to the carbon to which R^{1a} and R^{1b} are attached,
wherein each instance of R^{J1} is independently C₁₋₃ alkyl or C₁₋₄ haloalkyl,
wherein each instance of R^{J1a} is independently H, C₁₋₃ alkyl, C₁₋₄ haloalkyl;
or
R^{1a} and R^{1b}, together with the carbon atom to which they are attached form a C₃₋₆cycloalkyl, or a 3- to 6-membered monocyclic heterocycle containing 1 ring heteroatom selected from O, N and S, wherein the 1 ring heteroatom is not bonded to the carbon to which R^{1a} and R^{1b} are attached;
wherein each of said C₃₋₆ cycloalkyl and said 3- to 6-membered monocyclic heterocycle is unsubstituted or substituted with 1 or 2 substituents independently selected from halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, —(C(R^{J1a}₂))₀₋₂—NH2, —(C(R^{J1a}₂))₀₋₂—NHR^{J1}, and —(C(R^{J1a}₂))₀₋₂₋NR^{J1}₂, or wherein two geminal substituents, together with the carbon atom to which they are attached, form a C₃₋₆ cycloalkyl or 3- to 6-membered monocyclic heterocycle containing 1-2 heteroatoms selected from O, N, and S,
wherein each instance of R^{J1} is independently C₁₋₃ alkyl or C₁₋₄ haloalkyl,
wherein each instance of R^{J1a} is independently H, C₁₋₃ alkyl, C₁₋₄ haloalkyl;
R² is phenyl or 5- or 6-membered monocyclic heteroaryl having 1-3 ring heteroatoms independently selected from O, N and S,
wherein each of said phenyl and said 5- or 6-membered monocyclic heteroaryl is unsubstituted or substituted with 1-3 substituents independently selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J2a}₂))₀₋₂—SR^{J2}, —(C(R^{J2a}₂))₀₋₂—NH₂, —(C(R^{J2a}₂))₀₋₂—NHR^{J2}, —(C(R^{J2a}₂))₀₋₂—NR^{J2}₂, —C(O)R^{J1}₂, and —CN,
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein optionally methylenedioxy constitutes a substituent of said phenyl, wherein the methylene unit of the methylenedioxy is unsubstituted or substituted with halo; and
R³ is phenyl, or 5- or 6-membered monocyclic heteroaryl having 1-4 ring heteroatoms independently selected from O, N and S,
wherein each of said phenyl and said 5- or 6-membered monocyclic heteroaryl is unsubstituted or substituted with 1-3 substituents independently selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl;
each of R^{4a} and R^{4b} independently is —H, halo, C₁₋₄ alkyl and
Y is —NH— or —N(C₁₋₄ alkyl)-;
wherein 0 to 6 hydrogen atoms of said compound of Formula (I) are optionally replaced with deuterium; or

b. A method of treating a disease, disorder or condition in a subject comprising administering to the subject an effective amount of a chemical entity, which is a free compound of Formula (I) or a pharmaceutically acceptable salt thereof, wherein Formula (I) has the structure,

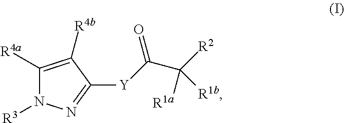

each of R^{1a} and R^{1b} independently is H, —C₁₋₄ alkyl, C₁₋₄ haloalkyl, —(C(R^{J1a}₂))₁₋₂—OH, —(C(R^{J1a}₂))₁₋₂—OR^{J1}, —(C(R^{J1a}₂))₁₋₂—SR^{J1}, —(C(R^{J1a}₂))₁₋₂—NH₂, —(C(R^{J1a}₂))₁₋₂—NHR¹, —(C(R^{J1a}₂))₁₋₂—NR^{J1}₂, C₃₋₆ cycloalkyl or a 3- to 6-membered monocyclic heterocycle containing 1 ring heteroatom selected from O, N, and S,
wherein the 3- to 6-membered monocyclic heterocycle does not contain a heteroatom bonded to the carbon to which R^{1a} and R^{1b} are attached,
wherein each instance of R^{J1} is independently C₁₋₃ alkyl or C₁₋₄ haloalkyl,
wherein each instance of R^{J1a} is independently H, C₁₋₃ alkyl, C₁₋₄ haloalkyl;
or
R^{1a} and R^{1b}, together with the carbon atom to which they are attached form a C₃₋₆cycloalkyl, or a 3- to 6-membered monocyclic heterocycle containing 1 ring heteroatom selected from O, N and S, wherein the 1 ring heteroatom is not bonded to the carbon to which R^{1a} and R^{1b} are attached;
wherein each of said C₃₋₆ cycloalkyl and said 3- to 6-membered monocyclic heterocycle is unsubstituted or substituted with 1 or 2 substituents independently selected from halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, —(C(R^{J1a}₂))₀₋₂—NH₂, —(C(R^{J1a}₂))₀₋₂—NHR^{J1}, and —(C(R^{J1a}₂))₀₋₂—NR^{J1}₂, or wherein two geminal substituents, together with the carbon atom to which they are attached, form a C₃₋₆ cycloalkyl or 3- to 6-membered monocyclic heterocycle containing 1-2 heteroatoms selected from O, N, and S,
wherein each instance of R^{J1} is independently C₁₋₃ alkyl or C₁₋₄ haloalkyl,
wherein each instance of R^{J1a} is independently H, C₁₋₃ alkyl, C₁₋₄ haloalkyl;
R² is phenyl or 5- or 6-membered monocyclic heteroaryl having 1-3 ring heteroatoms independently selected from O, N and S,
wherein each of said phenyl and said 5- or 6-membered monocyclic heteroaryl is unsubstituted or substituted with 1-3 substituents independently selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J1a}₂))₀₋₂—SR^{J1}, —(C(R^{J1a}₂))₀₋₂—NH₂, —(C(R^{J1a}₂))₀₋₂—NHR^{J1}, —(C(R^{J1a}₂))₀₋₂—NR^{J1}₂, —C(O)R^{J1}₂, and —CN,
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein optionally methylenedioxy constitutes a substituent of said phenyl, wherein the methylene unit of the methylenedioxy is unsubstituted or substituted with halo; and
R³ is phenyl, or 5- or 6-membered monocyclic heteroaryl having 1-4 ring heteroatoms independently selected from O, N and S,
wherein each of said phenyl and said 5- or 6-membered monocyclic heteroaryl is unsubstituted or substituted with 1-3 substituents independently selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J1a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, C₁₋₄ haloalkyl;
each of R^{4a} and R^{4b} independently is —H, halo, C₁₋₄ alkyl and
Y is —NH— or —N(C₁₋₄ alkyl)-;
wherein 0 to 6 hydrogen atoms of said compound of Formula (I) are optionally replaced with deuterium.
171. The method of embodiment 170, wherein R^{1a} and R^{1b}, together with the carbon atom to which they are attached form a C₃₋₆cycloalkyl, or a 3- to 6-membered monocyclic heterocycle containing 1 ring heteroatom selected from O, N and S, wherein the 1 ring heteroatom is not bonded to the carbon to which R^{1a} and R^{1b} are attached;
wherein each of said C₃₋₆ cycloalkyl and said 3- to 6-membered monocyclic heterocycle is unsubstituted or substituted with 1 or 2 substituents independently selected from halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, —(C(R^{J1a}₂))₀₋₂—NH2, —(C(R^{J1a}₂))₀₋₂—NHR^{J1}, and —(C(R^{J1a}₂))₀₋₂₋NR^{J1}₂, or wherein two geminal substituents, together with the carbon atom to which they are attached, form a C₄₋₆ cycloalkyl or 4- to 6-membered monocyclic heterocycle containing 1-2 heteroatoms selected from O, N, and S.
172. A method of treating a disease, disorder or condition in a subject comprising administering to the subject an effective amount of the chemical entity of any one of embodiments 1-168 or the pharmaceutical composition of embodiment 169.
173. The method of any one of embodiments 170-172, wherein the disease, disorder or condition is associated with one or more mutations of ABCD1 transporter protein.
174. The method of any one of embodiments 170-172, wherein the disease, disorder or condition is associated with impaired peroxisomal beta-oxidation.
175. The method of any one of embodiments 170-172, wherein the disease, disorder or condition associated with mutations of at least one of Acyl-CoA oxidase, D-Bifunctional protein, or ACBD5.
176. The method of any one of embodiments 170-172, wherein the disease, disorder or condition is associated with accumulation of very long chain fatty acid (VLCFA) levels.
177. The method of embodiment 176, wherein the VLCFA are 24 to 26 carbons long.
178. The method of embodiment 176, wherein the VLCFA are incorporation products.
179. A method of treating ALD comprising administering to a subject an effective amount of a chemical entity of any of embodiments 1-168 or the pharmaceutical composition of embodiment 169.
180. The method of embodiment 179, wherein ALD is the CALD phenotype.
181. The method of embodiment 179, wherein ALD is the AMN phenotype.
182. A method of reduction of very long chain fatty acids (VLCFA) levels in a subject comprising administering to the subject an effective amount of a chemical entity of any of embodiments 1-168 or a pharmaceutical composition of embodiment 169.
183. A method of reduction of very long chain fatty acids (VLCFA) levels in a biological sample of a subject comprising administering to the subject an effective amount of a chemical entity of any one of embodiments 1-168.
184. A method of reduction of a very long chain fatty acids (VLCFA) level in a cell comprising administering to the cell an effective amount of a chemical entity of any one of embodiments 1-168 or the pharmaceutical composition of embodiment 169.
185. A method of reduction of a very long chain fatty acids (VLCFA) level in the brain of a subject comprising administering systemically to the subject an effective amount of a chemical entity that penetrates the blood-brain-barrier to provide reduction in the VLCFA level in the brain of the subject.
186. The method of embodiment 185, wherein the VLCFA is VLCFA comprising at least 24 carbons.
187. The method of embodiment 185, wherein the VLCFA is VLCFA having 26 carbons.
188. The method of any one of embodiments 185-187, wherein the chemical entity is a chemical entity of any one of embodiments 1-168.
189. The method of any one of embodiments 185-188, wherein administering systemically to the subject comprises administering via oral administration, intravenous injection, or subcutaneous injection to the subject.
190. The method of any one of embodiments 185-188, wherein administering systemically to the subject comprises administering via oral administration to the subject.
191. The method of any one of embodiments 185-190, where in the reduction in a VLCFA level in the brain of the subject is at least about 30% when measured as a reduction in LPC 26:0 following administration of the chemical entity to the subject.
192. The method of embodiment 191, where in the reduction in LPC 26:0 following administration of the chemical entity to the subject is measured from a sample of cerebrospinal fluid (CSF) from the subject.
193. A method of preparing the chemical entity of any one of embodiments 1-168, comprising step (z): coupling a compound of formula:

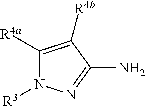

with a compound of formula:

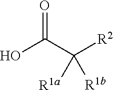

under conditions suitable to make the chemical entity.
194. The method of embodiment 193, wherein step (z) comprises converting the compound of formula:

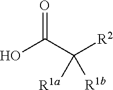

to a compound of formula:

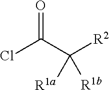

under conditions suitable to make the chemical entity; and
coupling the compound of formula:

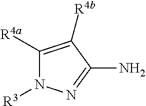

with the compound of formula:

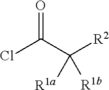

under conditions suitable to make the chemical entity.
195. The method of embodiment 193 or 194, further comprising, prior to step (z), step (y): coupling a compound of formula:

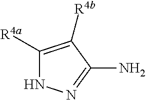

with a compound of formula R³—X, wherein X is a halide,
under conditions suitable to make the compound of formula:

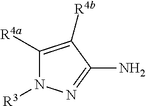

for use in step (z).
196. The method of embodiment 193 or 194, further comprising, prior to step (z), step (y): combining a compound of formula:

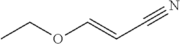

with a compound of formula:

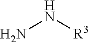

under conditions suitable to make the compound of formula:

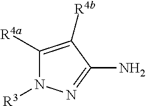

for use in step (z).
197. The method of embodiment 193 or 194, further comprising, prior to step (z), step (y): reducing a compound of formula:

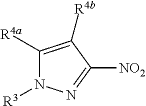

under conditions suitable to make the compound of formula:

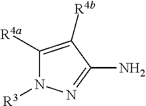

for use in step (z).
198. The method of embodiment 197, further comprising, prior to step (y), step (x): coupling a compound of formula:

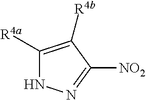

with a compound of formula R³—X, wherein X is a halide,
under conditions suitable to make the compound of formula:

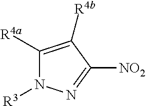

for use in step (y).
199. The method of any of embodiments 193-198, wherein in the chemical entity R^{1a} and R^{1b} together with the carbon atom to which they are attached form cyclopropyl,
further comprising, prior to step (z), step (w): combining a compound of formula:

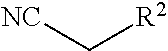

with a compound of formula:

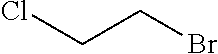

under conditions suitable to make the compound of formula:

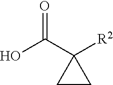

for use in step (z).
200. A method of preparing the chemical entity of embodiment 20, comprising step (z): coupling a compound of formula:

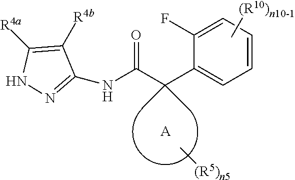

with a compound of formula R³—X, wherein X is a halide,
under conditions suitable to make the chemical entity.
201. The method of embodiment 200, wherein A is cyclopropyl and R² is phenyl, further comprising, prior to step (z), step (y): deprotecting a compound of formula:

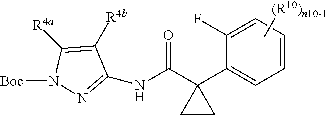

under conditions suitable to make the compound of formula:

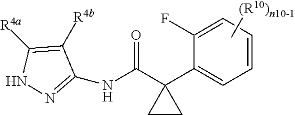

for use in step (z).
202. The method of embodiment 201, further comprising, prior to step (y), step (x): coupling a compound of formula:

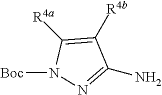

with a compound of formula:

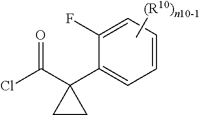

under conditions suitable to make the compound of formula:

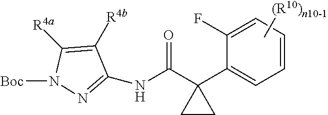

for use in step (y).
203. The method of embodiment 202, further comprising, prior to step (x), step (w): converting a compound of formula:

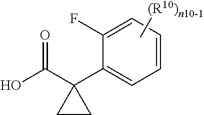

into a compound of formula:

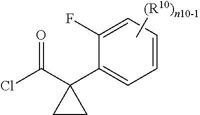

for use in step (x).

### EXAMPLES

### Example 1. Chemical Synthesis of Compounds Described Herein

1-substituted-pyrazol-3-amine intermediates (“pyrazole amine intermediates”) (Example 1.1) and acid intermediates (Example 1.2) were prepared separately and subsequently coupled using amide-bond formation methods (Example 1.3). Other compounds described herein were prepared using copper-mediated aryl coupling (Example 1.4), using SnAr (Example 1.5), using a boronic acid coupling sequence (Example 1.6), or using other methods (Example 1.7).

### Example 1.1. Pyrazole Amine Intermediates

Pyrazole amine intermediates were either commercially purchased (see Scheme Amine-1) or prepared as described below (see Schemes Amine-2, Amine-3, and Amine-4).

### Scheme Amine-1 (Commercially Purchased)

The following pyrazole amine intermediates were commercially available (Enamine, Monmouth Jct., N.J.):

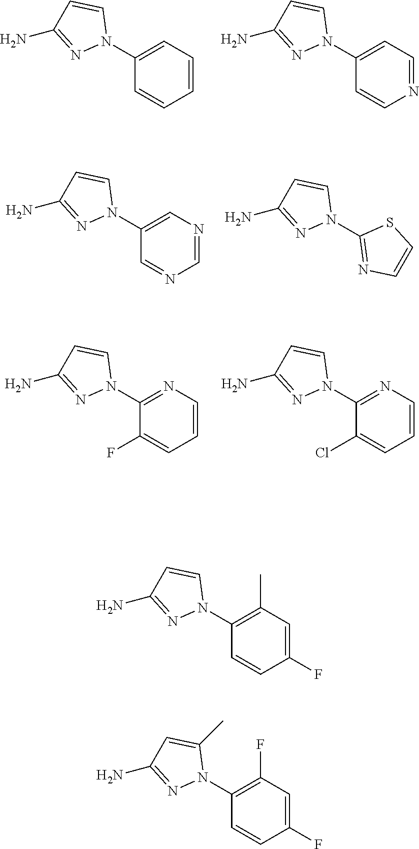

### Scheme Amine-2 (Copper Bromide Methods)

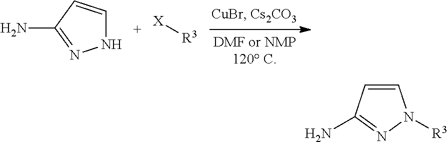

Scheme Amine-2, shown above, provides a general synthetic route for the preparation of 1-phenyl-pyrazol-3-amines and 1-heteroaryl-pyrazol-3-amines. Pyrazole amine intermediates within this section were synthesized using appropriate choice of aryl or heteroaryl halide (indicated with X—R3 in the scheme, wherein X is the halogen) following the procedures outlined below.

### 1-(5-fluoro-3-pyridyl)pyrazol-3-amine

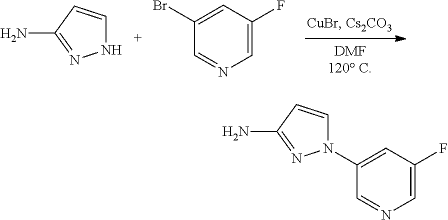 [SMILES: Nc1cc[nH]n1 ; Fc1cncc(Br)c1 ; Nc1ccn(-c2cncc(F)c2)n1]

1H-pyrazol-3-amine (1.0 g, 12.03 mmol), 3-bromo-5-fluoro-pyridine (2.3 g, 13.07 mmol), copper (I) bromide (100 mg, 0.70 mmol), and cesium carbonate (6 g, 18.42 mmol) were combined and suspended in NMP (10 mL). The mixture was heated in a sealed vessel at 120° C. for 12 h. Water (25 mL) and ethyl acetate (25 mL) were added. The resultant mixture was filtered through Celite, and the filter pad was rinsed with ethyl acetate (2×25 mL). The layers within the filtrate were separated, and the aqueous layer was extracted with ethyl acetate (25 mL). The combined organic fractions were washed with water (20 mL) and brine (20 mL), dried (Na₂SO₄), filtered, and concentrated. The crude residue was purified by silica gel chromatography (40 g silica column; linear gradient of 0-60% ethyl acetate/heptane). The resultant cream-colored solid was triturated with hot ethyl acetate/heptane to give 1-(5-fluoro-3-pyridyl)pyrazol-3-amine (298.9 mg, 13% yield) as a colorless crystalline solid. 1H NMR (400 MHz, DMSO-d₆) δ 8.82 (t, J=1.8 Hz, 1H), 8.32 (d, J=2.3 Hz, 1H), 8.29 (d, J=2.6 Hz, 1H), 7.93 (dt, J=10.8, 2.3 Hz, 1H), 5.84 (d, J=2.6 Hz, 1H), 5.33 (s, 2H) ppm. ESI-MS m/z calc. 178.06548, found 179.0 (M+1).

### 1-(6-chloropyridin-3-yl)-1H-pyrazol-3-amine

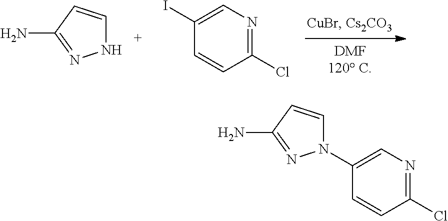

1H-pyrazol-3-amine (760 mg, 9.15 mmol), 2-chloro-5-iodo-pyridine (2.45 g, 10.23 mmol), copper (I) bromide (240 mg, 1.67 mmol) and cesium carbonate (4.5 g, 13.81 mmol) were combined and suspended in DMF (7.6 mL). The resultant reaction mixture was heated in a sealed vessel at 120° C. for 14 h. The reaction mixture was partitioned into 1:1 ethyl acetate/water. The layers were separated, and the aqueous phase was further extracted with ethyl acetate. The combined organics were dried (Na₂SO₄), filtered, and concentrated. Upon solvent removal, the product crystallized to provide 1-(6-chloropyridin-3-yl)-1H-pyrazol-3-amine (940 mg, 49% yield) as a black solid that was used without further purification. ESI-MS m/z calc. 194.04, found 195.02 (M+1).

### 1-(pyrazin-2-yl)-1H-pyrazol-3-amine)

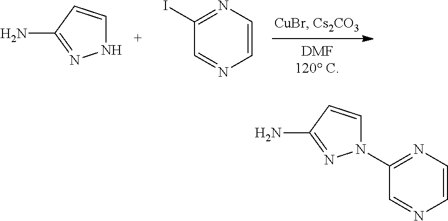 [SMILES: Nc1cc[nH]n1 ; Ic1cnccn1 ; Nc1ccn(-c2cnccn2)n1]

1H-pyrazol-3-amine (400 mg, 4.81 mmol), 2-iodopyrazine (1 g, 4.86 mmol), copper (I) bromide (136 mg, 0.95 mmol) and cesium carbonate (2 g, 6.14 mmol) were combined and suspended in DMF (6.0 mL). The resultant mixture was heated in a sealed vessel at 120° C. for 16 h. The reaction mixture was partitioned into 1:1 ethyl acetate/water and filtered through a plug of silica gel. The layers were separated, and the aqueous phase was further extracted with ethyl acetate (2×10 mL). The combined organics were washed with brine (20 mL) and water (20 mL), dried (Na₂SO₄), filtered, and concentrated. The crude residue was purified by silica gel chromatography (40 g silica gel column; linear gradient of 10-100% ethyl acetate/heptane) to provide 1-(pyrazin-2-yl)-1H-pyrazol-3-amine (263 mg, 33% yield) as a colorless solid. 1H NMR (400 MHz, DMSO-d₆) δ 8.88 (s, 1H), 8.38 (s, 2H), 8.26 (d, J=2.7 Hz, 1H), 5.90 (d, J=2.7 Hz, 1H), 5.47 (s, 2H) ppm. ESI-MS m/z calc. 161.07, found 162.53 (M+1).

### 1-(2-chloropyridin-4-yl)-1H-pyrazol-3-amine

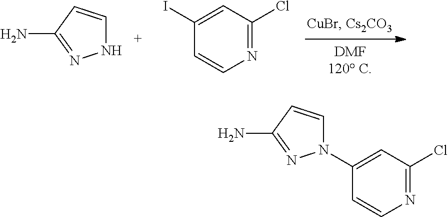 [SMILES: Nc1cc[nH]n1 ; Clc1cc(I)ccn1 ; Nc1ccn(-c2ccnc(Cl)c2)n1]

1H-pyrazol-3-amine (520 mg, 6.26 mmol), 2-chloro-4-iodo-pyridine (1.5 g, 6.27 mmol), copper (I) bromide (267 mg, 1.86 mmol), cesium carbonate (2.8 g, 8.59 mmol) were combined and suspended in DMF (6.0 mL) under nitrogen. The resultant reaction mixture was heated in a sealed vessel at 120° C. for 14 h. The reaction mixture was partitioned into 1:1 ethyl acetate/water (300 mL) and filtered through a plug of Celite. The layers were separated, and the aqueous further extracted with ethyl acetate. The combined organics were washed with brine, dried (Na₂SO₄), filtered, and concentrated. The crude residue was dissolved in ethanol/ethyl acetate/heptane (1:2:2) and hot-filtered through a glass frit. The resultant solution was stirred under a stream of nitrogen, and the desired product precipitated as the solvent evaporated. The product was then triturated with 20% ethyl acetate/heptane, filtered, and dried under vacuum to provide 1-(2-chloropyridin-4-yl)-1H-pyrazol-3-amine (766.5 mg, 60% yield). 1H NMR (300 MHz, DMSO-d₆) δ 8.60-8.15 (m, 2H), 7.80-7.54 (m, 2H), 5.90 (d, J=2.8 Hz, 1H), 5.51 (s, 2H) ppm. ESI-MS m/z calc. 194.04, found 195.06 (M+1).

### 1-(2-methylpyridin-4-yl)-1H-pyrazol-3-amine

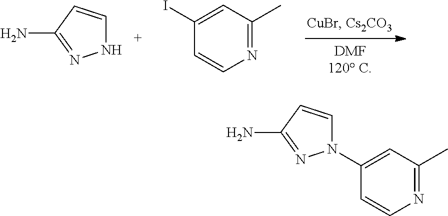

1H-pyrazol-3-amine (300 mg, 3.61 mmol), 4-iodo-2-methyl-pyridine (817 mg, 3.73 mmol), copper (I) bromide (60 mg, 0.42 mmol), cesium carbonate (1.3 g, 3.99 mmol) were combined in DMF (4.0 mL) and heated in a sealed vessel at 120° C. for 14 h. The reaction mixture was partitioned into 1:1 ethyl acetate/water and filtered through a plug of silica gel. The layers were separated, and the aqueous further extracted with ethyl acetate (2×10 mL). The combined organics were washed with brine, dried (Na₂SO₄), filtered, and concentrated. The crude residue was purified by silica gel chromatography (40 g silica gel column; linear gradient of 10-100% ethyl acetate/heptane) to provide 1-(2-methylpyridin-4-yl)-1H-pyrazol-3-amine (420 mg; 63% yield) as a colorless solid. 1H NMR (400 MHz, DMSO-d₆) δ 8.33 (d, J=5.6 Hz, 1H), 8.27 (d, J=2.7 Hz, 1H), 7.47 (d, J=2.1 Hz, 1H), 7.44-7.36 (m, 1H), 5.85 (d, J=2.7 Hz, 1H), 5.32 (s, 2H), 2.45 (s, 3H) ppm. ESI-MS m/z calc. 174.09, found 175.58 (M+1).

### 1-(2,5-difluoropyridin-4-yl)-1H-pyrazol-3-amine

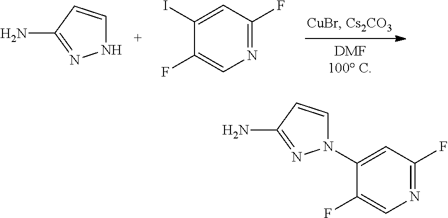 [SMILES: Nc1cc[nH]n1 ; Fc1cc(I)c(F)cn1 ; Nc1ccn(-c2cc(F)ncc2F)n1]

1H-pyrazol-3-amine (500 mg, 6.02 mmol), 2,5-difluoro-4-iodo-pyridine (1.450 g, 6.02 mmol), copper (I) bromide (300 mg, 2.09 mmol), and cesium carbonate (3.03 g, 9.30 mmol) were combined and suspended in DMF (5.1 mL). The resultant reaction mixture was heated in a sealed vessel at 100° C. for 42 h. The reaction mixture was partitioned into 1:1 ethyl acetate/water (150 mL) and filtered through a plug of Celite. The layers were separated, and the aqueous further extracted with ethyl acetate (100 mL). The combined organics were dried (Na₂SO₄), filtered, and concentrated. The crude residue was purified by silica gel chromatography (80 g silica gel column; linear gradient of 10-50% ethyl acetate/heptane) to provide 1-(2,5-difluoropyridin-4-yl)-1H-pyrazol-3-amine (263 mg, 20% yield). 1H NMR (400 MHz, DMSO-d₆) δ 8.27 (d, J=4.1 Hz, 1H), 8.06 (s, 1H), 7.34 (d, J=5.4 Hz, 1H), 5.99 (d, J=2.6 Hz, 1H), 5.61 (s, 2H) ppm. ESI-MS m/z calc. 196.06, found 197.10 (M+1).

### 1-(pyridin-2-yl)-1H-pyrazol-3-amine

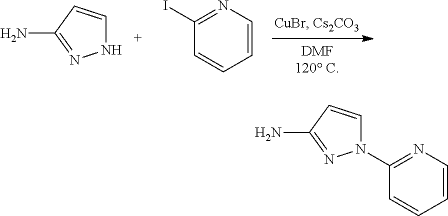 [SMILES: Nc1cc[nH]n1 ; Ic1ccccn1 ; Nc1ccn(-c2ccccn2)n1]

1H-pyrazol-3-amine (300 mg, 3.61 mmol), 2-iodopyridine (750 mg, 3.66 mmol), copper (I) bromide (60 mg, 0.42 mmol), and cesium carbonate (1.3 g, 3.99 mmol) were combined and suspended in DMF (4.0 mL). The resultant reaction mixture was heated in a sealed vessel at 120° C. for 14 h. The reaction mixture was partitioned into 1:1 ethyl acetate/water and filtered through a plug of silica gel. The layers were separated, and the aqueous further extracted with ethyl acetate (2×10 mL). The combined organics were washed with brine (20 mL) and water (20 mL), dried (Na₂SO₄), filtered, and concentrated. The crude residue was purified by silica gel chromatography (12 g silica gel column; linear gradient of 10-100% ethyl acetate/heptane) to provide 1-(pyridin-2-yl)-1H-pyrazol-3-amine (276 mg, 45% yield). 1H NMR (300 MHz, DMSO-d₆) δ 8.33 (d, J=4.1 Hz, 1H), 8.27 (d, J=2.6 Hz, 1H), 7.93-7.79 (m, 1H), 7.60 (d, J=8.2 Hz, 1H), 7.14 (dd, J=6.9, 5.2 Hz, 1H), 5.81 (d, J=2.6 Hz, 1H), 5.24 (s, 2H) ppm. ESI-MS m/z calc. 160.07, found 161.54 (M+1).

### 1-(6-methylpyridin-3-yl)-H-pyrazol-3-amine

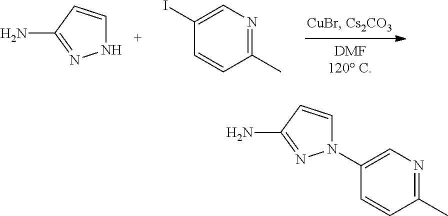

1H-pyrazol-3-amine (500 mg, 6.02 mmol), 5-iodo-2-methylpyridine (1.32 g, 6.12 mmol), copper (I) bromide (300 mg, 2.09 mmol) and cesium carbonate (3.03 g, 9.30 mmol were combined and suspended in DMF (5.0 mL). The resultant reaction mixture was heated in a sealed vessel at 120° C. for 24 h. The reaction mixture was partitioned into 1:1 ethyl acetate/water (150 mL) and filtered through a plug of Celite. The layers were separated, and the aqueous further extracted with ethyl acetate (3×50 mL). The combined organics were dried (Na₂SO₄), filtered, and concentrated to provide a regioisomeric mixture of products. The residue was purified twice by reverse phase chromatography: the first time using an ISCO 150 g C18 column and a linear gradient of 10-50% acetonitrile/water with TFA modifier, and the second time using an ISCO 150 g C18Aq column and a linear gradient of 0-70% acetonitrile/water with TFA modifier. The resultant TFA salt was dissolved in dichloromethane and washed with saturated aqueous NaHCO₃. The layers were separated, and the aqueous layer was further extracted with dichloromethane. The combined organics were dried (Na₂SO₄), filtered, and concentrated to provide 1-(6-methylpyridin-3-yl)-1H-pyrazol-3-amine (220 mg, 43% yield) as a colorless glass. 1H NMR (400 MHz, CDCl₃) δ 7.91 (d, J=2.6 Hz, 1H), 7.30 (d, J=2.4 Hz, 1H), 7.03 (dd, J=8.5, 2.7 Hz, 1H), 6.41 (d, J=8.3 Hz, 1H), 4.90 (d, J=2.4 Hz, 1H), 4.28 (s, 2H), 1.59 (s, 3H) ppm. ESI-MS m/z calc. 174.09, found 175.12 (M+1).

### 1-(3-chlorophenyl)-1H-pyrazol-3-amine

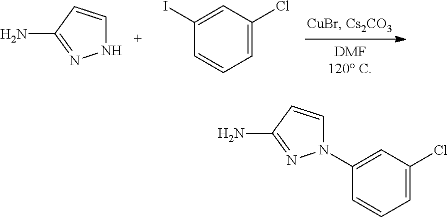 [SMILES: Nc1cc[nH]n1 ; Clc1cccc(I)c1 ; Nc1ccn(-c2cccc(Cl)c2)n1]

1H-pyrazol-3-amine (500 mg, 6.02 mmol) 1-chloro-3-iodo-benzene (800 μL, 6.46 mmol), copper (I) bromide (100 mg, 0.70 mmol) and cesium carbonate (3.0 g, 9.21 mmol) were combined and suspended in DMF (5.0 mL). The resultant reaction mixture was heated in a sealed vessel at 120° C. for 14 h. The reaction mixture was partitioned into 1:1 ethyl acetate/water. The layers were separated, and the aqueous further extracted with ethyl acetate (2×20 mL). The combined organics were dried (Na₂SO₄), filtered, and concentrated. The crude residue was purified by silica gel chromatography (40 g column; linear gradient of 0-30% ethyl acetate/heptane) to provide a solid which was further purified by crystallization from ethyl acetate/heptane. Material obtained from crystallization was purified once further by reverse phase chromatography (ISCO 150 g C18Aq column; linear gradient of 10-50% acetonitrile/water with TFA modifier). Pure fractions were washed with saturated sodium bicarbonate and extracted with ethyl acetate. The combined organic extracts were dried (Na₂SO₄), filtered, and concentrated to provide 1-(3-chlorophenyl)-1H-pyrazol-3-amine (300 mg, 25% yield). 1H NMR (400 MHz, DMSO-d₆) δ 8.21 (d, J=2.5 Hz, 1H), 7.72 (t, J=1.9 Hz, 1H), 7.61 (dd, J=8.3, 1.9 Hz, 1H), 7.40 (t, J=8.1 Hz, 1H), 7.15 (d, J=7.9 Hz, 1H), 5.77 (d, J=2.5 Hz, 1H) ppm. ESI-MS m/z calc. 193.04, found 194.03 (M+1).

### 1-(2-(trifluoromethyl)pyridin-4-yl)-1H-pyrazol-3-amine

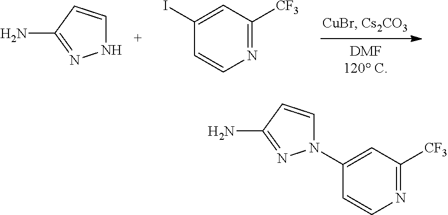 [SMILES: Nc1cc[nH]n1 ; FC(F)(F)c1cc(I)ccn1 ; Nc1ccn(-c2ccnc(C(F)(F)F)c2)n1]

1H-pyrazol-3-amine (500 mg, 6.02 mmol), 4-iodo-2-(trifluoromethyl)pyridine (1.84 g, 6.73 mmol), copper (I) bromide (150 mg, 1.05 mmol), and cesium carbonate (2.50 g, 7.68 mmol) were combined and suspended in DMF (5.0 mL). The resultant reaction mixture was heated in a sealed vessel at 120° C. under an atmosphere of nitrogen for 14 h. The reaction mixture was partitioned into 1:1 ethyl acetate/water (100 mL) and filtered through a plug of Celite. The layers were separated, and the aqueous further extracted with ethyl acetate (2×50 mL). The combined organics were washed with brine (2×100 mL), dried (Na₂SO₄), filtered, and concentrated. The crude residue was purified by silica gel chromatography (40 g silica gel column; linear gradient of 10-40% ethyl acetate/heptane) to provide 1-(2-(trifluoromethyl)pyridin-4-yl)-1H-pyrazol-3-amine (540 mg, 37% yield). ESI-MS m/z calc. 228.06, found 229.09 (M+1).

### 1-(3-fluorophenyl)-1H-pyrazol-3-amine

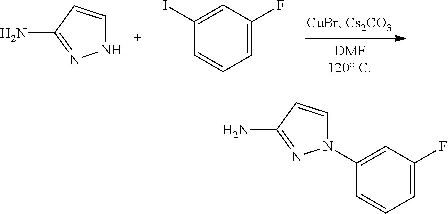 [SMILES: Nc1cc[nH]n1 ; Fc1cccc(I)c1 ; Nc1ccn(-c2cccc(F)c2)n1]

1H-pyrazol-3-amine (500 mg, 6.02 mmol), 1-fluoro-3-iodo-benzene (1.5 g, 6.76 mmol), copper (I) bromide (100 mg, 0.70 mmol), and cesium carbonate (3.0 g, 9.21 mmol) were combined and suspended in DMF (5.0 mL). The resultant reaction mixture was heated in a sealed vessel at 120° C. under an atmosphere of nitrogen for 14 h. The reaction mixture was partitioned into 1:1 ethyl acetate/water. The layers were separated, and the aqueous further extracted with ethyl acetate (2×20 mL). The combined organics were dried (Na₂SO₄), filtered, and concentrated. The crude residue was purified by silica gel chromatography (40 g silica gel column; linear gradient of 10-100% ethyl acetate/heptane) to provide 1-(3-fluorophenyl)pyrazol-3-amine (721.0 mg, 67% yield). 1H NMR (300 MHz, CDCl₃) δ 7.69 (d, J=2.6 Hz, 1H), 7.37 (dd, J=7.8, 5.7 Hz, 1H), 7.35 (s, 1H), 7.33 (s, 1H), 6.88 (dtd, J=8.5, 4.4, 2.8 Hz, 1H), 5.88 (d, J=2.6 Hz, 1H), 3.86 (s, 2H) ppm. ESI-MS m/z calc. 177.07022, found 178.05 (M+1).

### 1-(4-chlorophenyl)-H-pyrazol-3-amine

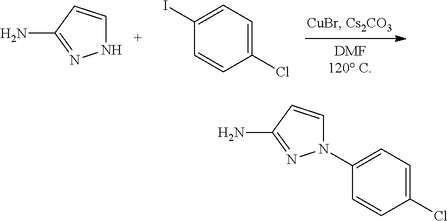

1H-pyrazol-3-amine (500 mg, 6.02 mmol), 1-chloro-4-iodo-benzene (1.5 g, 6.29 mmol), copper (I) bromide (100 mg, 0.70 mmol), and cesium carbonate (3.0 g, 9.21 mmol) were combined and suspended in DMF (5.0 mL). The resultant mixture was heated in a sealed vessel at 120° C. under an atmosphere of nitrogen for 14 h. The reaction mixture was partitioned into 1:1 ethyl acetate/water. The layers were separated, and the aqueous further extracted with ethyl acetate (2×20 mL). The combined organics were dried (Na₂SO₄), filtered, and concentrated. The crude residue was purified first by silica gel chromatography (40 g column, linear gradient of 0-30% ethyl acetate in heptane; material obtained was a mixture of regioisomers), and second by C18 reverse phase chromatography (10-50% acetonitrile/Water with TFA modifier). Pure fractions were washed with saturated sodium bicarbonate and extracted with ethyl acetate. The combined organic extracts were dried (Na₂SO₄), filtered, and concentrated to provide 1-(4-chlorophenyl)-1H-pyrazol-3-amine (542 mg, 57% yield). 1H NMR (300 MHz, DMSO-d₆) δ 8.15 (d, J=2.6 Hz, 1H), 7.79-7.58 (m, 2H), 7.52-7.27 (m, 2H), 5.75 (d, J=2.6 Hz, 1H), 5.14 (s, 2H) ppm. ESI-MS m/z calc. 193.04, found 194.03 (M+1).

### 5-fluoro-1-(5-fluoro-6-methoxypyridin-3-yl)-1H-pyrazol-3-amine

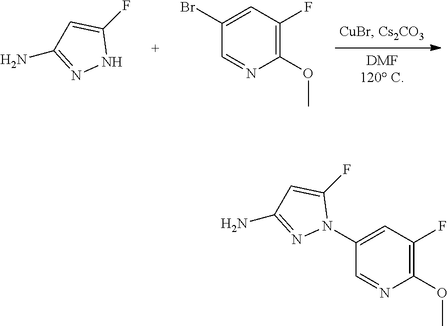

Prepared according to the procedure described above for 1-(4-chlorophenyl)-1H-pyrazol-3-amine except using 5-bromo-3-fluoro-2-methoxypyridine as a starting material. ESI-MS m/z calc. 226.07, found 227.07 (M+1).

### 1-(2-methoxypyrimidin-5-yl)pyrazol-3-amine

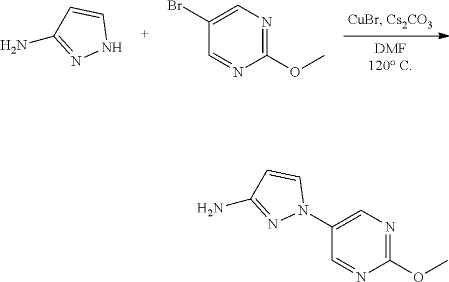

Prepared according to the procedure described above for 1-(4-chlorophenyl)-1H-pyrazol-3-amine except using 5-bromo-2-methoxy-pyrimidine as a starting material. ESI-MS m/z calc. 191.19, found 192.08 (M+1).

### 1-(1-methyl-1H-imidazol-4-yl)-1H-pyrazol-3-amine

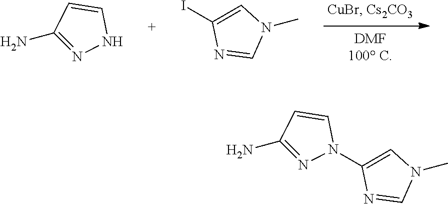

1H-pyrazol-3-amine (220 mg, 2.65 mmol), 4-iodo-1-methyl-imidazole (555 mg, 2.67 mmol), copper(I) bromide (38 mg, 0.265 mmol) cesium carbonate (900 mg, 2.76 mmol) and DMF (1.0 mL) were combined. The reaction vessel was sealed and stirred overnight at 100° C. The mixture was diluted with ethyl acetate and filtered though a layer of celite, and the filtrate was concentrated. The crude residue was purified by silica gel chromatography (linear gradient of 0-10% methanol/dichloromethane to provide 1-(1-methylimidazol-4-yl)pyrazol-3-amine (320 mg, 74% yield). 1H NMR (400 MHz, CDCl₃) δ 7.88 (d, J=2.5 Hz, 1H), 7.25 (d, J=1.6 Hz, 1H), 6.94 (d, J=1.7 Hz, 1H), 5.76 (d, J=2.5 Hz, 1H), 3.70 (d, J=4.0 Hz, 4H), 2.93 (d, J=28.7 Hz, 3H) ppm. ESI-MS m/z calc. 163.09, found 164.19 (M+1).

### 1-(1-methyl-1H-1,2,3-triazol-4-yl)-1H-pyrazol-3-amine

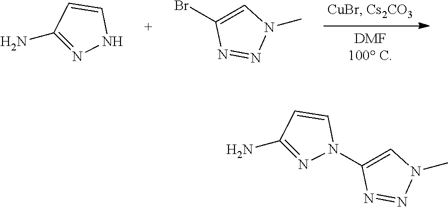

Prepared according to the procedure described above for 1-(1-methyl-1H-imidazol-4-yl)-1H-pyrazol-3-amine, except using 4-bromo-1-methyl-1H-1,2,3-triazole as a starting material. Product was obtained in 22% yield. 1H NMR (400 MHz, CDCl₃) δ 8.03 (d, J=2.6 Hz, 1H), 7.62 (s, 1H), 5.84 (d, J=2.6 Hz, 1H), 4.13 (s, 3H) ppm. ESI-MS m/z calc. 164.08, found 165.01 (M+1).

### 1-(2-(difluoromethoxy)pyridin-4-yl)-1H-pyrazol-3-amine

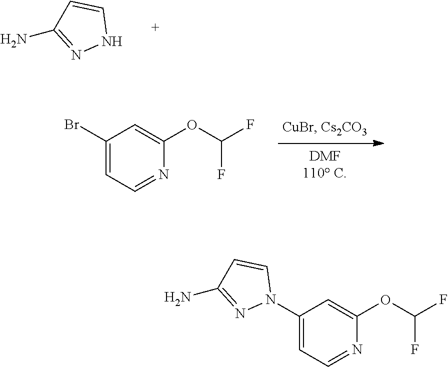 [SMILES: Nc1cc[nH]n1 ; FC(F)Oc1cc(Br)ccn1 ; Nc1ccn(-c2ccnc(OC(F)F)c2)n1]

1H-pyrazol-3-amine (200 mg, 2.41 mmol), 4-bromo-2-(difluoromethoxy)pyridine (539 mg, 2.41 mmol), cesium carbonate (784 mg, 2.41 mmol), copper(I) bromide (69 mg, 0.48 mmol) and DMF (2.0 mL) were combined under nitrogen. The vessel was sealed and heated to 110° C. for 16 h. The crude reaction mixture was filtered through Celite, washing filter pad with methanol. The filtrate was concentrated, and the residue was dissolve in dichloromethane and washed with 1N NaOH. The organics were collected and evaporated to provide 1-(2-(difluoromethoxy)pyridin-4-yl)-1H-pyrazol-3-amine, which was used without further manipulation. 1H NMR (400 MHz, DMSO-d₆) δ 8.36 (d, J=2.8 Hz, 1H), 8.16 (d, J=5.8 Hz, 1H), 7.52-7.48 (m, 1H), 7.21 (d, J=1.9 Hz, 1H), 5.89 (d, J=2.7 Hz, 1H), 5.47 (s, 2H) ppm.

### 5-(3-amino-1H-pyrazol-1-yl)-3-fluoropyridin-2-amine

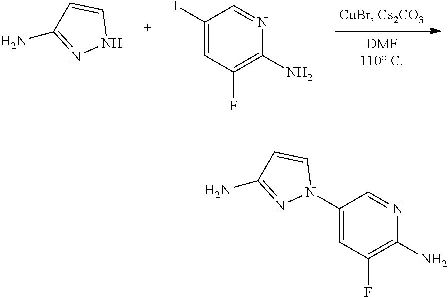 [SMILES: Nc1cc[nH]n1 ; Nc1ncc(I)cc1F ; Nc1ccn(-c2cnc(N)c(F)c2)n1]

Prepared according to the procedure described above for 1-(2-(difluoromethoxy)pyridin-4-yl)-1H-pyrazol-3-amine except using 3-fluoro-5-iodopyridin-2-amine as a starting material. Product was obtained in 60% yield. 1H NMR (400 MHz, DMSO-d₆) δ 8.36 (d, J=2.8 Hz, 1H), 8.16 (d, J=5.8 Hz, 1H), 7.52-7.48 (m, 1H), 7.21 (d, J=1.9 Hz, 1H), 5.89 (d, J=2.7 Hz, 1H), 5.47 (s, 2H) ppm.

### 1-(6-chloro-5-fluoropyridin-3-yl)-1H-pyrazol-3-amine

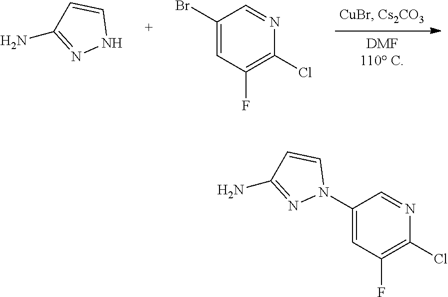 [SMILES: Nc1cc[nH]n1 ; Fc1cc(Br)cnc1Cl ; Nc1ccn(-c2cnc(Cl)c(F)c2)n1]

Prepared according to the procedure described above for 1-(2-(difluoromethoxy)pyridin-4-yl)-1H-pyrazol-3-amine except using 5-bromo-2-chloro-3-fluoropyridine as a starting material. Product was obtained in 45% yield. 1H NMR (400 MHz, DMSO-d6) δ 8.40 (m, 1H), 8.27 (m, 1H), 7.70-7.67 (m, 1H), 6.69 (d, J=2.7 Hz, 1H) ppm.

### 1-(2-(difluoromethyl)pyridin-4-yl)-1H-pyrazol-3-amine

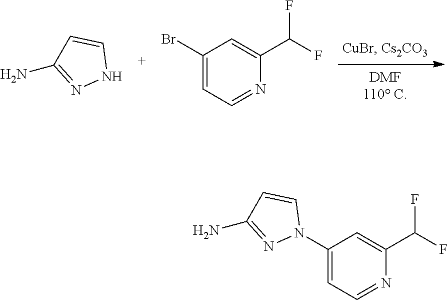 [SMILES: Nc1cc[nH]n1 ; FC(F)c1cc(Br)ccn1 ; Nc1ccn(-c2ccnc(C(F)F)c2)n1]

Prepared according to the procedure described above for 1-(2-(difluoromethoxy)pyridin-4-yl)-1H-pyrazol-3-amine except using 4-bromo-2-(difluoromethyl)pyridine as a starting material. Product was obtained in 69% yield. 1H NMR (400 MHz, DMSO-d6) δ 8.56 (d, J=5.6 Hz, 1H), 8.41 (d, J=2.8 Hz, 1H), 7.87 (d, J=2.1 Hz, 1H), 7.73 (m, 1H), 6.92 (t, J=55.0 Hz, 1H), 5.92 (d, J=2.7 Hz, 1H), 5.48 (s, 2H) ppm.

### 4-(3-amino-1H-pyrazol-1-yl)pyridin-2-amine

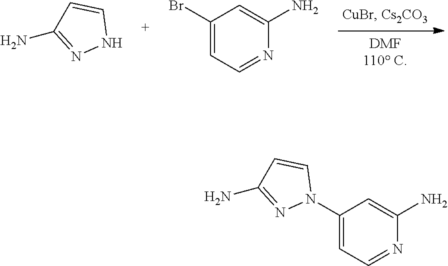 [SMILES: Nc1cc[nH]n1 ; Nc1cc(Br)ccn1 ; Nc1cc(-n2ccc(N)n2)ccn1]

Prepared according to the procedure described above for 1-(2-(difluoromethoxy)pyridin-4-yl)-1H-pyrazol-3-amine except using 4-(3-amino-1H-pyrazol-1-yl)pyridin-2-amine as a starting material. Product was obtained in 14% yield. 1H NMR (400 MHz, DMSO-d6) δ 8.09 (d, J=2.6 Hz, 1H), 7.81 (d, J=5.8 Hz, 1H), 6.76 (s, 1H), 6.66 (s, 1H), 5.95 (s, 2H), 5.77 (d, J=2.6 Hz, 1H), 5.20 (s, 2H) ppm.

### 5-(3-amino-1H-pyrazol-1-yl)-N,N-dimethylpyridin-2-amine

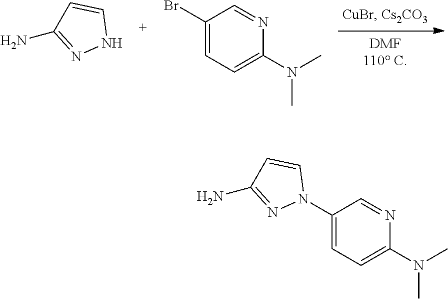

Prepared according to the procedure described above for 1-(2-(difluoromethoxy)pyridin-4-yl)-1H-pyrazol-3-amine except using 5-bromo-N,N-dimethylpyridin-2-amine as a starting material. Product was obtained in 63% yield.

### 5-(3-amino-1H-pyrazol-1-yl)-3-fluoro-N,N-dimethylpyridin-2-amine

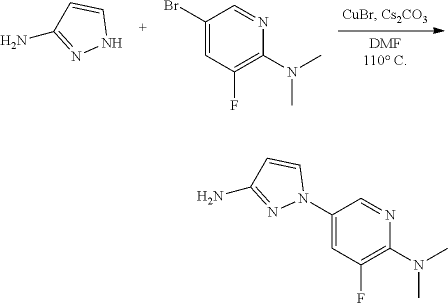 [SMILES: Nc1cc[nH]n1 ; CN(C)c1ncc(Br)cc1F ; CN(C)c1ncc(-n2ccc(N)n2)cc1F]

Prepared according to the procedure described above for 1-(2-(difluoromethoxy)pyridin-4-yl)-1H-pyrazol-3-amine except using 5-bromo-3-fluoro-N,N-dimethylpyridin-2-amine as a starting material. Product was obtained in 39% yield. 1H NMR (400 MHz, DMSO-d6) δ 8.31 (dd, J=2.3, 1.1 Hz, 1H), 8.05 (d, J=2.6 Hz, 1H), 7.79 (dd, J=14.6, 2.3 Hz, 1H), 5.71 (d, J=2.5 Hz, 1H), 5.09 (s, 2H), 2.97 (s, 6H) ppm.

### 4-(3-amino-1H-pyrazol-1-yl)-N,N-dimethylpyridin-2-amine

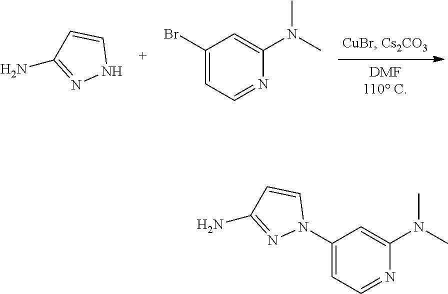

Prepared according to the procedure described above for 1-(2-(difluoromethoxy)pyridin-4-yl)-1H-pyrazol-3-amine except using 4-bromo-N,N-dimethylpyridin-2-amine as a starting material. Product was obtained in 59% yield. 1H NMR (400 MHz, DMSO-d₆) δ 8.38 (d, J=2.8 Hz, 1H), 7.94 (d, J=2.5 Hz, 1H), 7.77 (dd, J=9.1, 2.8 Hz, 1H), 6.69 (d, J=9.2 Hz, 1H), 5.66 (d, J=2.4 Hz, 1H), 4.95 (s, 2H), 3.02 (s, 6H) ppm.

### 5-(3-amino-1H-pyrazol-1-yl)-1-methylpyridin-2(1H)-one

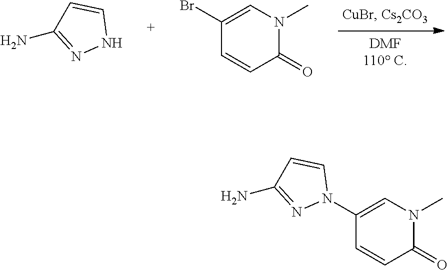

Prepared according to the procedure described above for 1-(2-(difluoromethoxy)pyridin-4-yl)-1H-pyrazol-3-amine except using 5-bromo-1-methylpyridin-2(1H)-one as a starting material. 1H NMR (400 MHz, Benzene-d₆) δ 8.55 (d, J=2.7 Hz, 1H), 8.20-8.11 (m, 2H), 7.16 (d, J=8.9 Hz, 1H), 5.78 (d, J=2.7 Hz, 1H), 4.06 (s, 2H), 3.57 (s, 3H) ppm.

### 1-(6-(difluoromethoxy)pyridin-3-yl)-1H-pyrazol-3-amine

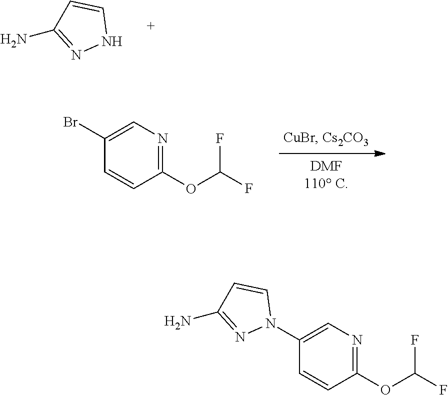 [SMILES: Nc1cc[nH]n1 ; FC(F)Oc1ccc(Br)cn1 ; Nc1ccn(-c2ccc(OC(F)F)nc2)n1]

Prepared according to the procedure described above for 1-(2-(difluoromethoxy)pyridin-4-yl)-1H-pyrazol-3-amine except using 5-bromo-2-(difluoromethoxy)pyridine as a starting material. 1H NMR (400 MHz, Benzene-d₆) δ 8.09 (d, J=2.7 Hz, 1H), 7.62-7.52 (m, 1H), 7.47 (d, J=3.5 Hz, 1H), 7.30 (d, J=3.5 Hz, 1H), 5.84 (d, J=2.7 Hz, 1H), 5.40 (s, 1H), 4.06 (s, 2H) ppm.

### 1-(2-chlorothiazol-5-yl)-1H-pyrazol-3-amine

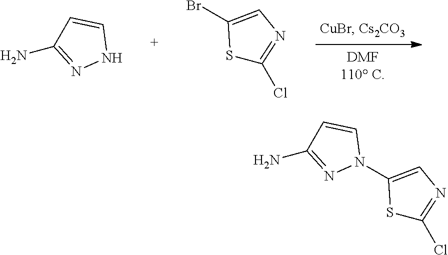 [SMILES: Nc1cc[nH]n1 ; Clc1ncc(Br)s1 ; Nc1ccn(-c2cnc(Cl)s2)n1]

Prepared according to the procedure described above for 1-(2-(difluoromethoxy)pyridin-4-yl)-1H-pyrazol-3-amine except using 5-bromo-2-chlorothiazole as a starting material. 1H NMR (400 MHz, Benzene-d6) δ 8.24 (d, J=2.7 Hz, 1H), 8.18 (d, J=5.2 Hz, 1H), 5.81 (d, J=2.7 Hz, 1H), 5.32 (s, 2H) ppm.

### 1-(3-methoxypyridin-4-yl)-1H-pyrazol-3-amine

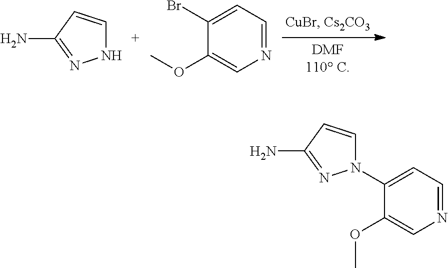

Prepared according to the procedure described above for 1-(2-(difluoromethoxy)pyridin-4-yl)-1H-pyrazol-3-amine except using 4-bromo-3-methoxypyridine as a starting material. 1H NMR (400 MHz, Benzene-d6) δ 8.27 (d, J=2.7 Hz, 1H), 7.88 (d, J=2.4 Hz, 1H), 7.65 (dd, J=9.0, 2.8 Hz, 1H), 6.48 (d, J=9.2 Hz, 2H), 5.64 (d, J=2.4 Hz, 1H), 4.91 (s, 2H), 2.89 (s, 3H) ppm.

### 1-(2-methoxythiazol-5-yl)-1H-pyrazol-3-amine

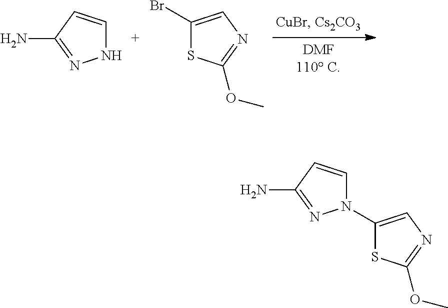

Prepared according to the procedure described above for 1-(2-(difluoromethoxy)pyridin-4-yl)-1H-pyrazol-3-amine except using 5-bromo-2-methoxythiazole as a starting material.

### 5-(3-amino-1H-pyrazol-1-yl)-N-methylpyridin-2-amine

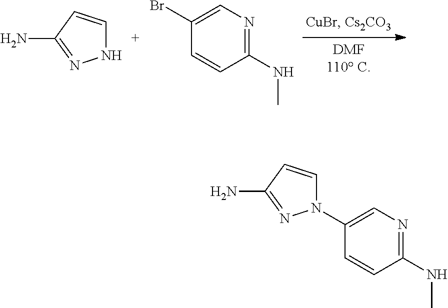

Prepared according to the procedure described above for 1-(2-(difluoromethoxy)pyridin-4-yl)-1H-pyrazol-3-amine except using 5-bromo-N-methylpyridin-2-amine as a starting material. ESI-MS m/z calc. 189.10, found 190.10 (M+1).

### 1-(2,4-dimethylthiazol-5-yl)-1H-pyrazol-3-amine

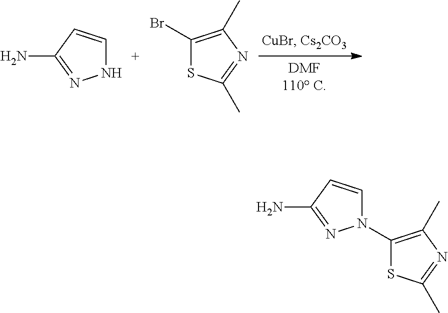

Prepared according to the procedure described above for 1-(2-(difluoromethoxy)pyridin-4-yl)-1H-pyrazol-3-amine except using 5-bromo-2,4-dimethylthiazole as a starting material.

### 1-(2-methyl-1,2,4-triazol-3-yl)pyrazol-3-amine

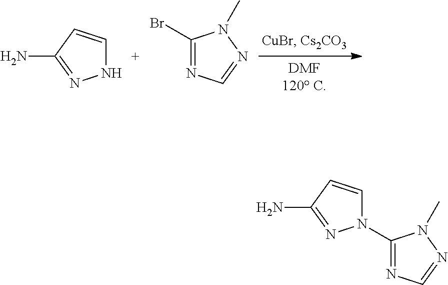

1H-pyrazol-3-amine (305 mg, 3.671 mmol, 1.0 eq), 5-bromo-1-methyl-1,2,4-triazole (600 mg, 3.704 mmol, 1.01 eq), copper(I) bromide (106 mg, 0.739 mmol, 0.2 eq), cesium carbonate (1.26 g, 3.852 mmol, 1.05 eq), and N,N-dimethylformamide (2.2 mL) were combined. The reaction vessel was sealed and stirred overnight at 120° C. The mixture was diluted with dichloromethane and methanol, and the mixture was filtered though a layer of Celite. The filtrate was concentrated. The crude residue was purified by silica gel chromatography (linear gradient of 0-15% methanol/dichloromethane) to provide 1-(2-methyl-1,2,4-triazol-3-yl)pyrazol-3-amine (118 mg, 19% yield). 1H NMR (400 MHz, CDCl₃) δ 7.99 (d, J=2.7 Hz, 1H), 7.68 (s, 1H), 5.89 (d, J=2.7 Hz, 1H), 4.18 (s, 3H), 3.91 (s, 2H) ppm. ESI-MS m/z calc. 164.08, found 165.23 (M+1).

### 1-[1-(difluoromethyl)-3-methyl-pyrazol-4-yl]pyrazol-3-amine

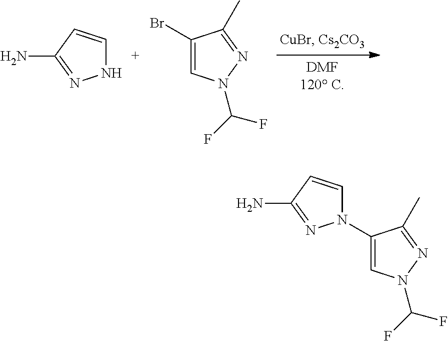

Prepared according to the procedure described above for 1-(2-methyl-1,2,4-triazol-3-yl)pyrazol-3-amine, except using 4-bromo-1-(difluoromethyl)-3-methyl-1H-pyrazole as a starting material. Product was obtained in 9% yield. 1H NMR (400 MHz, CDCl₃) δ 7.89 (s, 1H), 7.37 (d, J=2.4 Hz, 1H), 7.09 (t, J=60.7 Hz, 1H), 5.79 (d, J=2.5 Hz, 1H), 3.91-3.66 (m, 2H), 2.38 (d, J=0.9 Hz, 3H) ppm. ESI-MS m/z calc. 213.08, found 214.17 (M+1).

### 1-isoxazol-4-ylpyrazol-3-amine

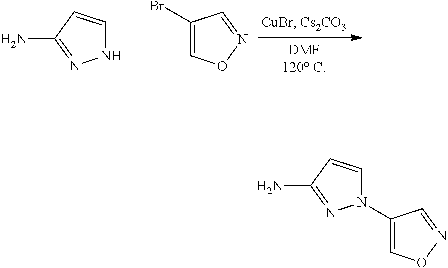 [SMILES: Nc1cc[nH]n1 ; Brc1cnoc1 ; Nc1ccn(-c2cnoc2)n1]

Prepared according to the procedure described above for 1-(2-methyl-1,2,4-triazol-3-yl)pyrazol-3-amine, except using 4-bromoisoxazole as a starting material. Product was obtained in 2% yield. 1H NMR (400 MHz, Methanol-d₄) δ 8.08 (d, J=5.3 Hz, 1H), 8.03 (d, J=2.3 Hz, 1H), 6.43 (d, J=2.3 Hz, 1H), 6.14 (d, J=5.3 Hz, 1H), 4.40 (s, 3H) ppm.

### 1-(1-methyl-1,2,4-triazol-3-yl)pyrazol-3-amine

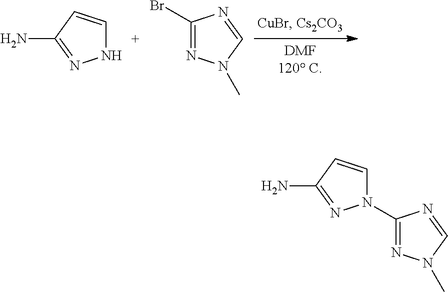

Prepared according to the procedure described above for 1-(2-methyl-1,2,4-triazol-3-yl)pyrazol-3-amine, except using 3-bromo-1-methyl-1,2,4-triazole as a starting material. Product was obtained in 13% yield. 1H NMR (400 MHz, Chloroform-d) δ 7.96 (d, J=2.6 Hz, 1H), 7.89 (d, J=0.7 Hz, 1H), 5.84 (d, J=2.6 Hz, 1H), 3.91 (d, J=0.6 Hz, 5H) ppm. ESI-MS m/z calc. 164.08, found 165.23 (M+1).

### 1-isoxazol-3-ylpyrazol-3-amine

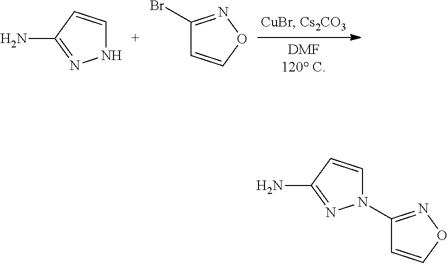 [SMILES: Nc1cc[nH]n1 ; Brc1ccon1 ; Nc1ccn(-c2ccon2)n1]

Prepared according to the procedure described above for 1-(2-methyl-1,2,4-triazol-3-yl)pyrazol-3-amine, except using 3-bromoisoxazole as a starting material. Product was obtained in 10% yield. 1H NMR (400 MHz, CDCl₃) δ 8.21 (d, J=5.0 Hz, 1H), 8.05 (d, J=2.3 Hz, 1H), 6.53 (d, J=2.3 Hz, 1H), 6.08 (d, J=5.0 Hz, 1H), 5.70 (s, 2H) ppm.

### 1-(2-methylpyrazol-3-yl)pyrazol-3-amine

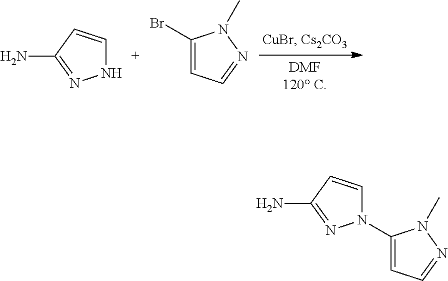

Prepared according to the procedure described above for 1-(2-methyl-1,2,4-triazol-3-yl)pyrazol-3-amine, except using 5-bromo-1-methyl-pyrazole as a starting material. Product was obtained in 15% yield. 1H NMR (400 MHz, CDCl₃) δ 7.45 (d, J=2.0 Hz, 1H), 7.38 (d, J=2.5 Hz, 1H), 6.18 (d, J=2.0 Hz, 1H), 5.85 (d, J=2.5 Hz, 1H), 3.88 (s, 3H), 3.82 (s, 2H) ppm. ESI-MS m/z calc. 163.09, found 164.19 (M+1).

### 1-(1-methyl-1H-imidazol-5-yl)-1H-pyrazol-3-amine

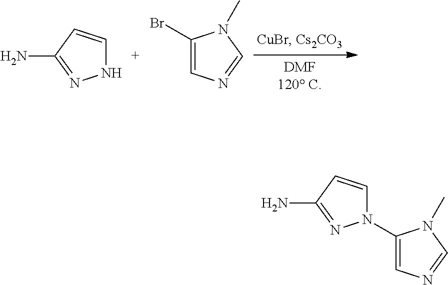

Prepared according to the procedure described above for 1-(2-methyl-1,2,4-triazol-3-yl)pyrazol-3-amine, except using 5-bromo-1-methyl-imidazole as a starting material. Product was obtained in 16% yield. 1H NMR (400 MHz, CDCl₃) δ 7.41 (s, 1H), 7.33 (d, J=2.4 Hz, 1H), 7.02 (d, J=1.1 Hz, 1H), 5.81 (d, J=2.4 Hz, 1H), 3.78 (s, 2H), 3.57 (s, 3H) ppm. ESI-MS m/z calc. 163.09, found 164.19 (M+1).

### 1-(4-methyl-4H-1,2,4-triazol-3-yl)-1H-pyrazol-3-amine

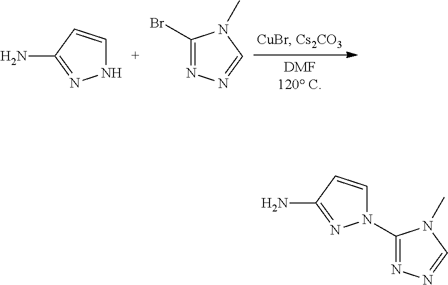

Prepared according to the procedure described above for 1-(2-methyl-1,2,4-triazol-3-yl)pyrazol-3-amine, except using 3-bromo-4-methyl-1,2,4-triazole as a starting material. Product was obtained in 14% yield. 1H NMR (400 MHz, CDCl3/Methanol-d4) δ 8.29 (s, 1H), 7.90 (d, J=2.7 Hz, 1H), 5.97 (d, J=2.8 Hz, 1H), 5.64 (d, J=2.3 Hz, 2H), 3.90 (s, 3H) ppm. ESI-MS m/z calc. 164.08, found 165.18 (M+1).

### 1-(5-methyl-1,3,4-oxadiazol-2-yl)pyrazol-3-amine

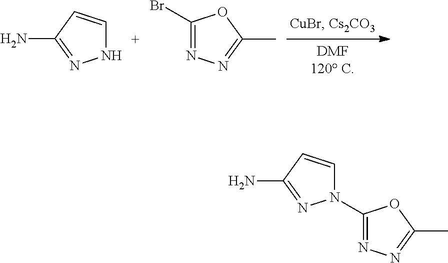

Prepared according to the procedure described above for 1-(2-methyl-1,2,4-triazol-3-yl)pyrazol-3-amine, except using 2-bromo-5-methyl-1,3,4-oxadiazole as a starting material. Product was obtained in 17% yield. 1H NMR (400 MHz, Chloroform-d) δ 7.97 (d, J=2.8 Hz, 1H), 5.97 (d, J=2.9 Hz, 1H), 4.06 (s, 2H), 2.56 (s, 3H) ppm. ESI-MS m/z calc. 165.07, found 166.17 (M+1).

### 1-(3-fluoropyridin-4-yl)-1H-pyrazol-3-amine

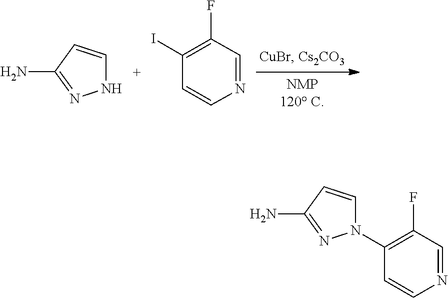 [SMILES: Nc1cc[nH]n1 ; Fc1cnccc1I ; Nc1ccn(-c2ccncc2F)n1]

1H-pyrazol-3-amine (500 mg, 6.02 mmol), 3-fluoro-4-iodo-pyridine (1.5 g, 6.73 mmol), copper (I) bromide (100 mg, 0.70 mmol), and cesium carbonate (3.0 g, 9.21 mmol) were combined and suspended in NMP (7.0 mL). The resultant mixture was heated in a sealed vessel at 120° C. under an atmosphere of nitrogen for 18 h. The reaction mixture was partitioned into 1:1 ethyl acetate/water. The layers were separated, and the aqueous further extracted with ethyl acetate (2×20 mL). The combined organics were dried (Na₂SO₄), filtered, and concentrated. The crude residue was purified by reverse phase chromatography (ISCO C18 Aq 150 g column; linear gradient of 10-50% acetonitrile in water with TFA modifier). Pure fractions were washed with saturated sodium bicarbonate and extracted with dichloromethane. The combined organic extracts were dried (Na₂SO₄), filtered, and concentrated to provide a yellow solid. The solid was further purified by trituration with warm ethyl acetate/heptane to provide 1-(3-fluoropyridin-4-yl)-1H-pyrazol-3-amine (431 mg; 48% yield) as a yellow powder. 1H NMR (300 MHz, DMSO-d₆) δ 8.70 (d, J=5.1 Hz, 1H), 8.42 (d, J=5.6 Hz, 1H), 8.07 (t, J=2.5 Hz, 1H), 7.82 (dd, J=7.5, 5.6 Hz, 1H), 6.00 (d, J=2.8 Hz, 1H), 5.44 (s, 2H) ppm. ESI-MS m/z calc. 178.07, found 179.00 (M+1).

### 1-(pyridazin-4-yl)-1H-pyrazol-3-amine

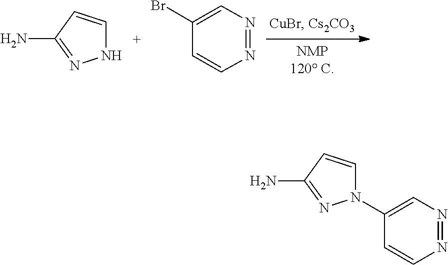 [SMILES: Nc1cc[nH]n1 ; Brc1ccnnc1 ; Nc1ccn(-c2ccnnc2)n1]

1H-pyrazol-3-amine (650 mg, 7.82 mmol), 4-bromopyridazine (1.5 g, 9.40 mmol), copper (I) bromide (100 mg, 0.70 mmol), and cesium carbonate (5.0 g, 15.35 mmol) were combined and suspended in NMP (9.0 mL). The resultant mixture was heated in a sealed vessel at 120° C. under an atmosphere of nitrogen for 60 h. The reaction mixture was partitioned into 1:1 ethyl acetate/water. The layers were separated, and the aqueous further extracted with ethyl acetate. The combined organics were dried (Na₂SO₄), filtered, and concentrated. The crude residue was purified by reverse phase chromatography (ISCO C18 Aq 150 g column; linear gradient of 10-50% acetonitrile in water with TFA modifier) to provide 1-(pyridazin-4-yl)-1H-pyrazol-3-amine (as TFA salt in 93% purity; 1.2 g, 51% yield) as a yellow solid. ESI-MS m/z calc. 161.07, found 162.02 (M+1).

### 1-(thiazol-5-yl)-1H-pyrazol-3-amine

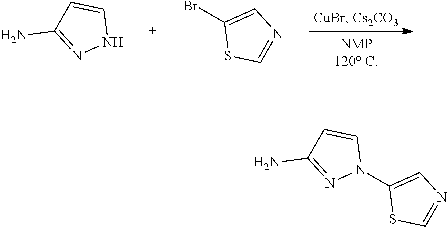 [SMILES: Nc1cc[nH]n1 ; Brc1cncs1 ; Nc1ccn(-c2cncs2)n1]

1H-pyrazol-3-amine (600 mg, 7.22 mmol), 5-bromothiazole (1.30 g, 7.93 mmol), copper (I) bromide (240 mg, 1.67 mmol), and cesium carbonate (4.0 g, 12.28 mmol) were combined and suspended in NMP (6.0 mL). The resultant mixture was heated in a sealed vessel at 120° C. under an atmosphere of nitrogen for 60 h. The reaction mixture was partitioned into 1:1 ethyl acetate/brine. The layers were separated, and the aqueous further extracted with ethyl acetate. The combined organics were dried (Na₂SO₄), filtered, and concentrated. The crude residue was purified by silica gel chromatography (40 g column, linear gradient of 0-50% ethyl acetate/heptane) to provide 1-(thiazol-5-yl)-1H-pyrazol-3-amine (55 mg, 4% yield). ESI-MS m/z calc. 166.03, found 166.93 (M+1).

### 1′-methyl-1′H-[1,3′-bipyrazol]-3-amine

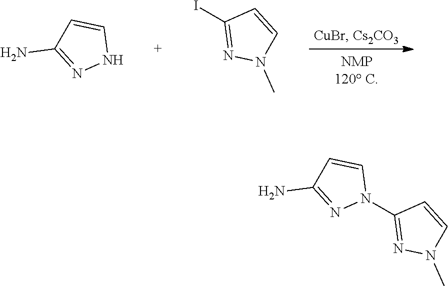

To a solution of 3-iodo-1-methyl-1H-pyrazole (4.0 g, 19.23 mmol) in NMP (60 mL) was added 1H-pyrazol-3-amine (1.6 g, 19.23 mmol), copper (I) bromide (3.0 g, 21 mmol) and cesium carbonate (15.6 g, 48.07 mmol). The resultant mixture was heated in a sealed vessel at 120° C. under an atmosphere of nitrogen for 8 h. The reaction mixture was partitioned into 1:1 ethyl acetate/brine. The layers were separated, and the aqueous further extracted with ethyl acetate. The combined organics were dried (Na₂SO₄), filtered, and concentrated to provide 1′-methyl-1′H-[1,3′-bipyrazol]-3-amine (2.0 g, 64% yield) as a brown oil which was used without further purification.

### 4-(3-amino-1H-pyrazol-1-yl)pyridin-2-ol

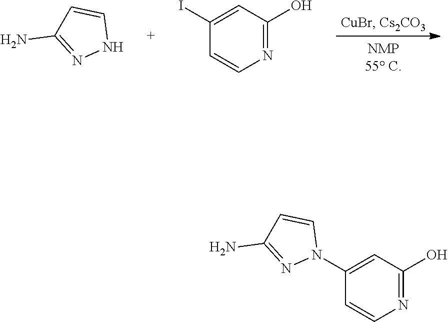 [SMILES: Nc1cc[nH]n1 ; Oc1cc(I)ccn1 ; Nc1ccn(-c2ccnc(O)c2)n1]

1H-pyrazol-3-amine (250 mg, 3.01 mmol), 4-iodopyridin-2-ol (700 mg, 3.17 mmol), copper (I) bromide (50 mg, 0.35 mmol), and cesium carbonate (1.7 g, 5.22 mmol) were combined in NMP (2.5 mL). The reaction mixture was heated to 55° C. for 16 h. The reaction mixture was partitioned into 1:1 ethyl acetate/brine, and the resultant biphasic mixture was filtered through Celite. The layers were separated, and the aqueous further extracted with 10% methanol/ethyl acetate. The combined organics were dried (Na₂SO₄), filtered, and concentrated. The crude residue was purified by reverse phase chromatography (ISCO C18 Aq 150 g column; linear gradient of 0-30% acetonitrile in water with TFA modifier) to provide 4-(3-amino-1H-pyrazol-1-yl)pyridin-2-ol (TFA salt; 35.2 mg, 4% yield). ESI-MS m/z calc. 176.07, found 176.97 (M+1).

### 1-(2-methyl pyrimidin-5-yl)-1H-pyrazol-3-amine

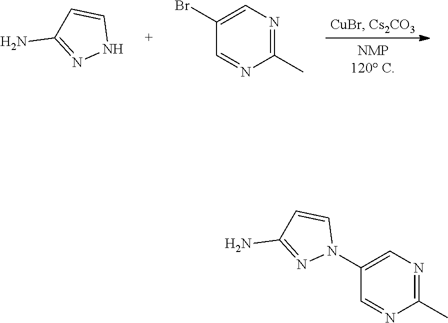

1H-pyrazol-3-amine (440 mg, 5.30 mmol), 5-bromo-2-methyl-pyrimidine (1.0 g, 5.78 mmol), copper (I) bromide (80 mg, 0.56 mmol), and cesium carbonate (2.4 g, 7.37 mmol) were combined and suspended in NMP (6.0 mL). The resultant mixture was heated in a sealed vessel under nitrogen at 120° C. for 16 h. The reaction mixture was partitioned into 1:1 ethyl acetate/water. The layers were separated, and the aqueous further extracted with ethyl acetate (2×25 mL). The combined organics were washed with brine (20 mL), dried (Na₂SO₄), filtered, and concentrated to yield an orange crystalline solid of 90% purity. The solid was triturated with ethyl acetate/heptane to provide 1-(2-methylpyrimidin-5-yl)-1H-pyrazol-3-amine (303.9 mg, 31% yield). 1H NMR (300 MHz, DMSO-d₆) δ 8.98 (d, J=2.0 Hz, 2H), 8.25 (d, J=2.6 Hz, 1H), 5.82 (d, J=2.6 Hz, 1H), 5.30 (s, 2H), 2.60 (d, J=1.8 Hz, 3H) ppm. ESI-MS m/z calc. 175.09, found 176.07 (M+1).

### 1-(2-methyl pyrimidin-5-yl-4,6-d₂)-1H-pyrazol-3-amine

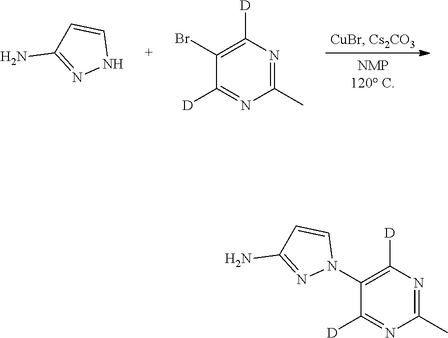

1H-pyrazol-3-amine (300 mg, 3.61 mmol), 5-bromo-4,6-dideuterio-2-methyl-pyrimidine (690 mg, 3.94 mmol), copper (I) bromide (100 mg, 0.70 mmol) and cesium carbonate (1.7 g, 5.22 mmol) were combined and suspended in NMP (5.0 mL). The resultant reaction mixture was heated in a sealed vessel under nitrogen at 120° C. for 16 h. The reaction mixture was partitioned into 1:1 ethyl acetate/water. The layers were separated, and the aqueous further extracted with ethyl acetate (2×25 mL). The combined organics were washed with brine (20 mL), dried (Na₂SO₄), filtered, and concentrated to furnish a crude product which was triturated with ethyl acetate/heptane to provide 1-(2-methylpyrimidin-5-yl-4,6-d₂)-1H-pyrazol-3-amine (170.8 mg, 30% yield) as a brick-red powder. ESI-MS m/z calc. 177.10, found 178.10 (M+1).

### 1-(3,5-difluorophenyl)-1H-pyrazol-3-amine

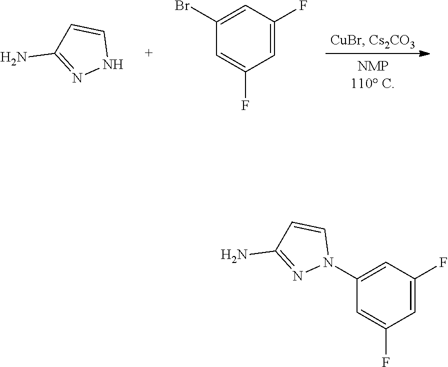 [SMILES: Nc1cc[nH]n1 ; Fc1cc(F)cc(Br)c1 ; Nc1ccn(-c2cc(F)cc(F)c2)n1]

1H-pyrazol-3-amine (500 mg, 6.02 mmol), 1-bromo-3,5-difluoro-benzene (1.4 g, 7.3 mmol), copper (I) bromide (215 mg, 0.96 mmol), and cesium carbonate (3.5 g, 11.00 mmol) were combined and suspended in NMP (5.0 mL). The resultant reaction mixture was heated in a sealed vessel under nitrogen at 110° C. for 5 h. The reaction mixture was partitioned into ethyl acetate and water. The layers were separated, and the aqueous further extracted with ethyl acetate (2×25 mL). The combined organics were washed with brine (20 mL), dried (Na₂SO₄), filtered, and concentrated. The crude product was purified by silica gel chromatography (linear gradient of 10-20% ethyl acetate/heptane) to provide 1-(3,5-difluorophenyl)-1H-pyrazol-3-amine (374.3 mg, 37% yield) 1H NMR (400 MHz, DMSO-d₆) δ 8.22 (d, J=2.6 Hz, 1H), 7.36 (dd, J=9.2, 1.8 Hz, 2H), 6.98-6.88 (m, 1H), 5.82 (d, J=2.4 Hz, 1H), 5.27 (s, 2H) ppm. ESI-MS m/z calc. 195.06, found 196.50 (M+1).

### 1-(5-chloro-3-pyridyl)pyrazol-3-amine

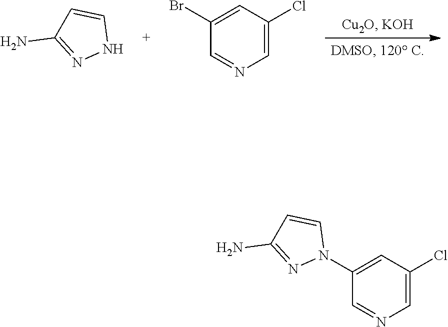 [SMILES: Nc1cc[nH]n1 ; Clc1cncc(Br)c1 ; Nc1ccn(-c2cncc(Cl)c2)n1]

1H-pyrazol-3-amine (1.7 g, 20.5 mmol, 1.0 eq), 3-bromo-5-chloropyridine (5.9 g, 30.8 mmol, 1.5 eq), cuprous oxide (300 mg, 2.1 mmol, 0.1 eq), potassium hydroxide (2.3 g, 41.0 mmol, 2.0 eq), and anhydrous DMSO (80 mL) were combined and heated at 120° C. for 12 h under an atmosphere of argon. The mixture was poured into 200 mL of water and extracted with ethyl acetate (3×100 mL). The organic layer was dried (Na₂SO₄), filtered, and concentrated. The residue was purified by silica gel chromatography (isocratic 1:1 ethyl acetate/heptane) to provide an impure product. The material was further purified by reverse phase HPLC (acetonitrile/water with NH₄HCO₃ modifier) to provide 1-(5-chloro-3-pyridyl)pyrazol-3-amine (1.0 g, 25.1%).

### Scheme Amine-3 (Hydrazine Method)

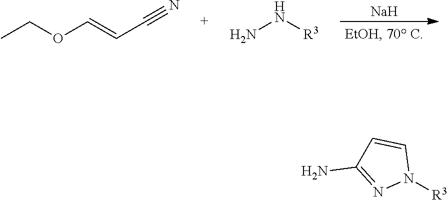

Scheme Amine-3, shown above, provides a general synthetic route for the preparation of 1-phenyl-pyrazol-3-amines and 1-heteroaryl-pyrazol-3-amines. Pyrazole amine intermediates within this section were synthesized using appropriate choice of aryl or heteroaryl hydrazine following the procedures outlined below.

### 1-(2-fluorophenyl)-1H-pyrazol-3-amine

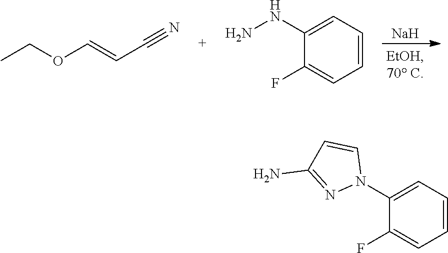

To a 0° C. solution of (2-fluorophenyl)hydrazine (3.0 g, 23.8 mmol) in ethanol (40 mL) was added 3-ethoxyacrylonitrile (4.6 g, 47.6 mmol, 2.0 eq) and NaH (60% dispersion in oil, 3.8 g, 85.2 mmol, 4.0 eq). The mixture was stirred at 70° C. for 2 h. The reaction mixture was partitioned between ethyl acetate and water. The layers were separated, and the organic layer was washed with brine, dried (Na₂SO₄), filtered, and concentrated. The crude residue was purified by silica-gel chromatography (linear gradient of 10-33% ethyl acetate/heptane) to provide 1-(2-fluorophenyl)-1H-pyrazol-3-amine.

### 1-(4-fluorophenyl)-1H-pyrazol-3-amine

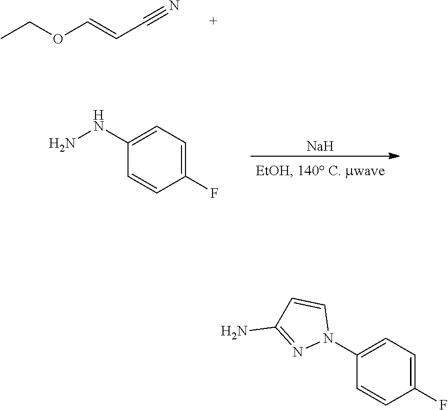

Sodium hydride (320 mg, 8.0 mmol) was added in portions to ethanol (10 mL) at room temperature. After stirring for 5 minutes, this sodium ethoxide solution was added to a slurry of (4-fluorophenyl)hydrazine (hydrochloride salt; 0.50 g, 3.08 mmol) and 3-ethoxyacrylonitrile (320 μL, 3.11 mmol) in ethanol (8.0 mL). The resultant reaction mixture was heated to 140° C. in the microwave for 30 min. After cooling, the reaction mixture was partitioned into ethyl acetate and water. The layers were separated, and the organics were dried (Na₂SO₄), filtered, and concentrated to an oil. The crude material was purified by silica chromatography (40 g silica column; linear gradient of 0-60% ethyl acetate/heptane) to provide 1-(4-fluorophenyl)-1H-pyrazol-3-amine (90 mg, 16.5% yield) as a yellow solid. ESI-MS m/z calc. 177.07, found 178.01 (M+1).

### 1-(pyridin-3-yl)-1H-pyrazol-3-amine

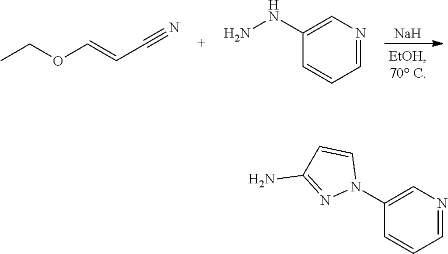

To a 0° C. solution of 3-hydrazinylpyridine (2.0 g, 18.34 mmol) in ethanol (40 mL) was added 3-ethoxyacrylonitrile (3.56 g, 36.70 mmol, 2.0 eq) and NaH (60% dispersion in oil; 2.9 g, 73.4 mmol, 4.0 eq). The mixture was warmed to room temperature and then heated to 70° C. for 2 h. The reaction mixture was partitioned between brine and THF. The layers were separated, and the organic layer was washed with brine, dried (Na₂SO₄), filtered, and concentrated. The crude residue was purified by silica-gel chromatography (linear gradient of 1.0-2.5% methanol/dichloromethane) to provide 1-(pyridin-3-yl)-1H-pyrazol-3-amine (500 mg, 17% yield) as a yellow oil (mixture of products).

### 1-(3-(trifluoromethyl)phenyl)-1H-pyrazol-3-amine

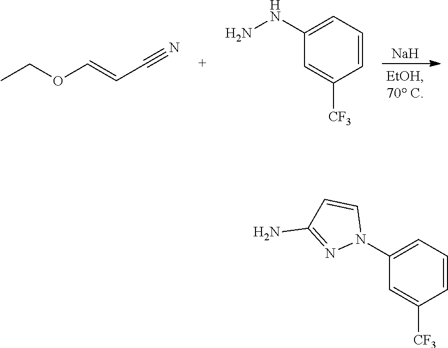

Prepared according to the procedure described for 1-(2-fluorophenyl)-1H-pyrazol-3-amine, except using (3-(trifluoromethyl)phenyl)hydrazine as a starting material.

### 1-(2,5-difluorophenyl)-1H-pyrazol-3-amine

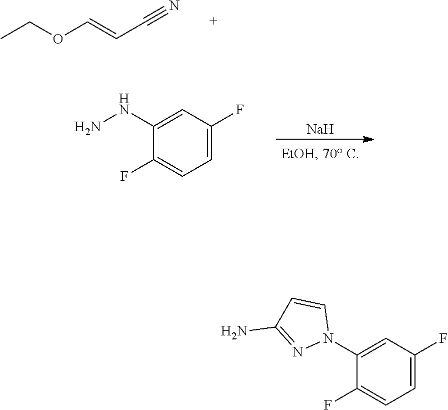

Prepared according to the procedure described for 1-(2-fluorophenyl)-1H-pyrazol-3-amine, except using (2,5-difluorophenyl)hydrazine as a starting material.

### 1-(4-(trifluoromethyl)phenyl)-1H-pyrazol-3-amine

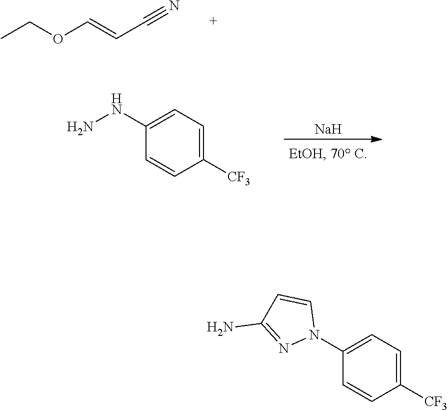

Prepared according to the procedure described for 1-(2-fluorophenyl)-1H-pyrazol-3-amine, except using (4-(trifluoromethyl)phenyl)hydrazine as a starting material.

### 1-(3,4-difluorophenyl)-1H-pyrazol-3-amine

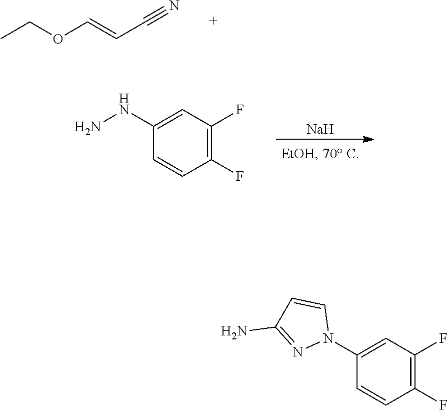

Prepared according to the procedure described for 1-(2-fluorophenyl)-1H-pyrazol-3-amine, except using (3,4-difluorophenyl)hydrazine as a starting material.

### 1-(4-chloro-3-fluorophenyl)-1H-pyrazol-3-amine

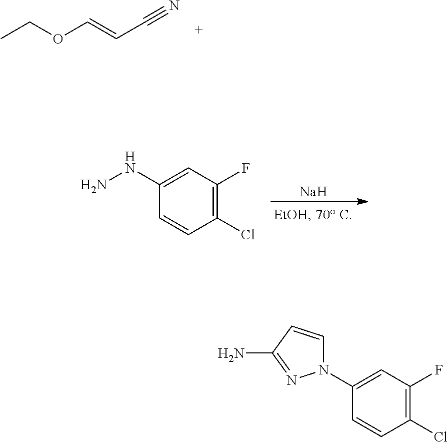

Prepared according to the procedure described for 1-(2-fluorophenyl)-1H-pyrazol-3-amine, except using (4-chloro-3-fluorophenyl)hydrazine as a starting material.

### 1-(3-chloro-4-fluorophenyl)-1H-pyrazol-3-amine

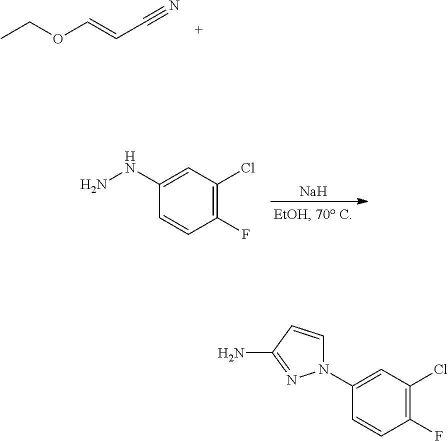

Prepared according to the procedure described for 1-(2-fluorophenyl)-1H-pyrazol-3-amine, except using (3-chloro-4-fluorophenyl)hydrazine as a starting material.

### Scheme Amine-4 (Multistep Via Nitro Methods)

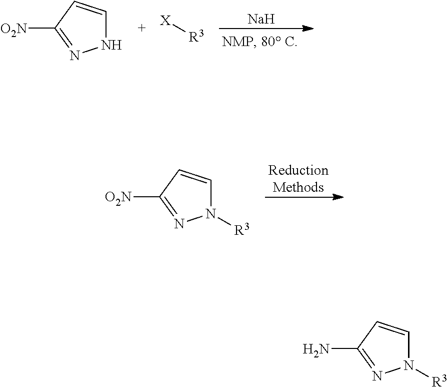

Scheme Amine-4, shown above, provides a general synthetic route for the preparation of 1-phenyl-pyrazol-3-amines and 1-heteroaryl-pyrazol-3-amines. Pyrazole amine intermediates within this section were synthesized using appropriate choice of aryl or heteroaryl halide following the procedures outlined below.

### Example: 1-(pyrimidin-4-yl)-1H-pyrazol-3-amine

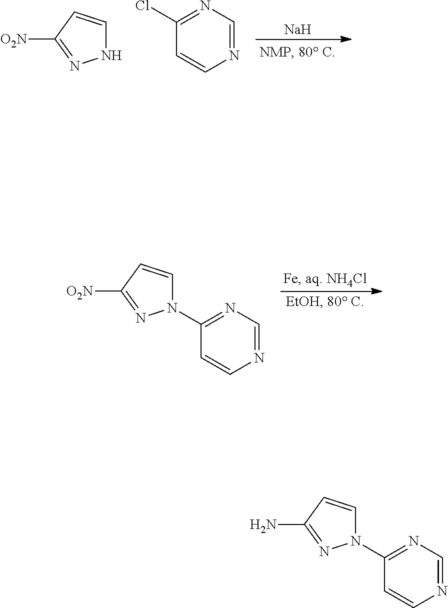

### Step 1: 4-(3-nitropyrazol-1-yl)pyrimidine

To a 0° C. solution of 3-nitro-1H-pyrazole (1.5 g, 13.27 mmol) in NMP (12.0 mL) was added NaH (1.2 g of 60% w/w, 30.00 mmol). After 20 min, gas evolution slowed and reaction mixture was allowed to warm slowly to room temperature. The mixture was cooled back to 0° C., and 4-chloropyrimidine (hydrochloride salt; 2.2 g, 14.57 mmol) was added. The resultant reaction mixture was heated to 80° C. and stirred for 60 h. The reaction mixture was poured over ice with swirling, and a colorless precipitate formed. After standing for 16 h, the mixture was filtered, and the peach-colored solids were air-dried. The material was dissolved in hot ethyl acetate and then diluted with heptane to 50% ethyl acetate/heptane. The solution was chilled on ice, and the precipitated solid was collected by vacuum filtration and washed with heptanes to provide 4-(3-nitropyrazol-1-yl)pyrimidine (1.92 g, 74% yield). 1H NMR (400 MHz, DMSO-d₆) δ 9.24 (d, J=1.3 Hz, 1H), 9.05 (d, J=5.6 Hz, 1H), 8.98 (d, J=2.9 Hz, 1H), 8.06 (dd, J=5.6, 1.3 Hz, 1H), 7.41 (d, J=2.9 Hz, 1H) ppm. ESI-MS m/z calc. 191.04, found 192.00 (M+1).

### Step 2: 1-(pyrimidin-4-yl)-1H-pyrazol-3-amine

4-(3-nitro-1H-pyrazol-1-yl)pyrimidine (1.88 g, 9.6 mmol) was dissolved in ethanol (50 mL) at room temperature. To the resultant solution was added aqueous ammonium chloride (8 mL of 7 M, 56.00 mmol) and iron (3.0 g, 53.72 mmol). The resultant mixture was stirred 6 h at 80° C. and 16 h at room temperature. The reaction mixture was filtered through Celite, and the filter pad was washed with ethanol and ethyl acetate. The combined filtrate was concentrated to a white solid. The solid was dissolved in dichloromethane and dried (Na₂SO₄). After filtration, the solvent was evaporated to provide 1-(pyrimidin-4-yl)-1H-pyrazol-3-amine as an orange solid (849.6 mg, 54% yield). 1H NMR (400 MHz, DMSO-d₆) δ 8.87 (d, J=1.3 Hz, 1H), 8.68 (d, J=5.7 Hz, 1H), 8.34 (d, J=2.8 Hz, 1H), 7.51 (dd, J=5.7, 1.3 Hz, 1H), 5.94 (d, J=2.8 Hz, 1H), 5.61 (s, 2H) ppm. ESI-MS m/z calc. 161.07, found 161.98 (M+1).

### Example: 1-(2-methoxypyridin-4-yl)-1H-pyrazol-3-amine

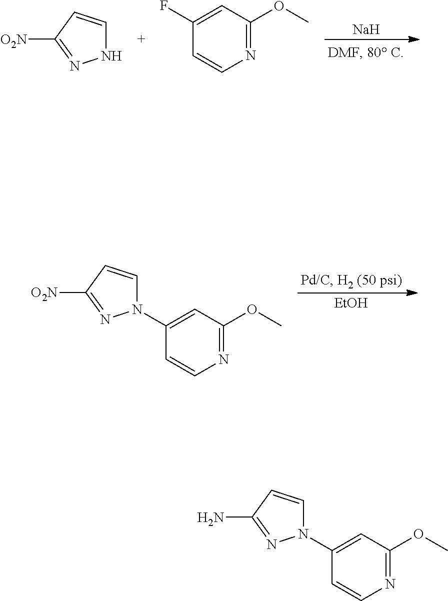

### Step 1: 2-methoxy-4-(3-nitro-1H-pyrazol-1-yl)pyridine

To a 0° C. solution of 3-nitro-1H-pyrazole (1.0 g, 8.84 mmol) in DMF (8.0 mL) was added NaH (450 mg of 60% w/w, 11.25 mmol). After 20 minutes, the mixture was warmed to room temperature and stirred a further 60 min. 4-fluoro-2-methoxy-pyridine (1.29 g, 10.15 mmol) was added, and the resultant reaction mixture was stirred for 16 h at room temperature followed by 80° C. for 6 h. The reaction mixture was poured over ice, and a colorless precipitate formed. The product was collected by vacuum filtration, and the solids air-dried to provide 2-methoxy-4-(3-nitro-1H-pyrazol-1-yl)pyridine (692 mg, 35% yield). 1H NMR (300 MHz, DMSO-d₆) δ 8.97 (t, J=2.4 Hz, 1H), 8.36 (dd, J=5.7, 1.9 Hz, 1H), 7.59 (dt, J=5.7, 1.9 Hz, 1H), 7.41 (dt, J=6.6, 2.0 Hz, 2H), 3.94 (d, J=1.9 Hz, 3H) ppm. ESI-MS m/z calc. 220.06, found 221.08 (M+1).

### Step 2: 1-(2-methoxypyridin-4-yl)-1H-pyrazol-3-amine

To a pressure vessel containing Pd/C (65 mg of 10% w/w, 0.06 mmol) suspended in ethanol (20.0 mL) was added 2-methoxy-4-(3-nitro-1H-pyrazol-1-yl)pyridine (680 mg, 3.06 mmol). The resultant solution was shaken under 50 psi of H₂ gas for 48 h. The mixture was filtered through Celite, and the filtrate concentrated. The crude residue was purified by silica gel chromatography (12 g silica column; linear gradient 0-50% ethyl acetate/heptane) to provide 1-(2-methoxypyridin-4-yl)-1H-pyrazol-3-amine (410 mg, 69% yield) as a colorless solid. 1H NMR (300 MHz, DMSO-d₆) δ 8.28 (t, J=2.7 Hz, 1H), 8.08 (dd, J=5.8, 2.1 Hz, 1H), 7.26 (dt, J=5.8, 1.9 Hz, 1H), 6.97 (t, J=2.1 Hz, 1H), 5.84 (t, J=2.8 Hz, 1H), 5.33 (s, 2H) ppm. ESI-MS m/z calc. 190.09, found 191.06 (M+1)⁺.

### Example: 1-(2-fluoropyridin-4-yl)-1H-pyrazol-3-amine

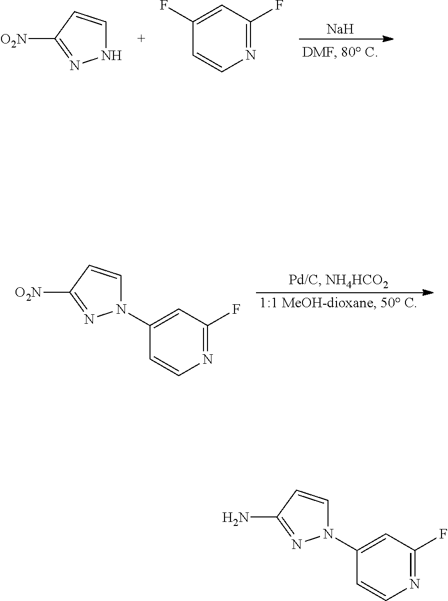

### Step 1: 2-fluoro-4-(3-nitro-1H-pyrazol-1-yl)pyridine

To a 0° C. solution of 3-nitro-1H-pyrazole (250.0 g, 2.17 mol, 1.0 eq) in anhydrous DMF (2.5 L; 10.2 vol eq) under nitrogen was added NaH (95.42 g of 60% w/w, 2.39 mol, 1.1 eq) in batches over 30 min while maintaining temperature below 8° C. The mixture was stirred for 1 h then 2,4-difluoropyridine (300 mL, 3.29 mol, 1.5 eq) was added, and the reaction was warmed to room temperature and stirred for approximately 16 hours (h). The reaction mixture was diluted with water (12.5 L) and stirred vigorously for 1 h. The off-white solid was collected by vacuum filtration. The solid was re-suspended in water (2 L) and filtered, and this step was repeated once further. The product was dried under vacuum, then suspended in heptane (4 L), stirred 3 h at room temperature, and filtered. The solid was washed with two further portions of heptane (2 L each) and dried under vacuum to provide 2-fluoro-4-(3-nitro-1H-pyrazol-1-yl)pyridine (426.3 g of 92% purity, 87% yield). 1H NMR (400 MHz, DMSO-d6) δ 9.01 (d, J=2.8 Hz, 1H), 8.45 (d, J=5.7 Hz, 1H), 7.95 (ddd, J=5.7, 1.9, 1.2 Hz, 1H), 7.81 (t, J=1.4 Hz, 1H), 7.46 (d, J=2.8 Hz, 1H) ppm. ESI-MS m/z calc. 208.04, found 209.01 (M+1).

### Step 2: 1-(2-fluoropyridin-4-yl)-1H-pyrazol-3-amine

A mixture of 2-fluoro-4-(3-nitropyrazol-1-yl)pyridine (200.0 g, 893.6 mol, 1.0 eq), 10% Pd/C (18.60 g of 10% w/w, 17.48 mmol, 0.02 eq), ammonium formate (572.95 g, 8.814 mol, 10 eq), methanol (500 mL; 2.7 vol eq), and dioxane (1.0 L; 5.4 vol eq) was stirred at 50° C. until starting materials were consumed, which was about 2.5 h. The reaction mixture was hot-filtered through Celite, and the filter cake was washed with dioxane (500 mL) and methanol (250 mL). The combined filtrate was concentrated to a white solid. The solid was suspended in water (3 L), stirred overnight (about 16 h), and filtered. Water (1 L) was added, mixture stirred, filtered, and dried on vac line for about 6 h. The product was dried at 55° C. under vacuum overnight to provide 1-(2-fluoropyridin-4-yl)-1H-pyrazol-3-amine (145.0 g, 89% yield). 1H NMR (400 MHz, DMSO-d6) δ 8.35 (d, J=2.8 Hz, 1H), 8.14 (d, J=5.8 Hz, 1H), 7.56 (dt, J=5.7, 1.7 Hz, 1H), 7.28 (d, J=1.8 Hz, 1H), 5.91 (d, J=2.8 Hz, 1H), 5.47 (s, 2H) ppm. ESI-MS m/z calc. 178.07, found 178.98 (M+1).

### Example: 1-(2-fluoropyridin-4-yl)-1H-pyrazol-3-amine (alternate synthesis)

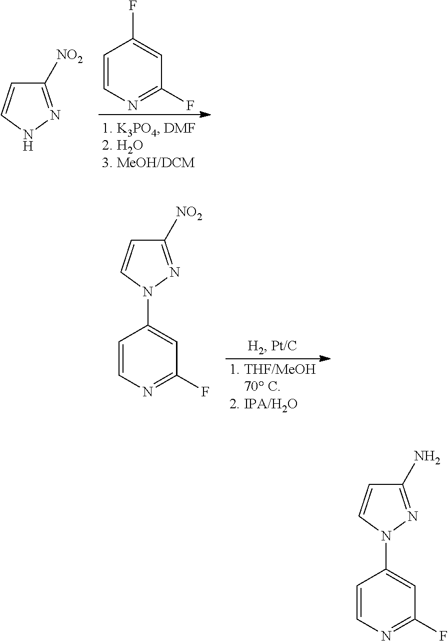

### Step 1: 2-fluoro-4-(3-nitro-1H-pyrazol-1-yl)pyridine

A reactor was charged with 3-nitro-1H-pyrazole (300 g, 2.67 mol, limiting reagent). Anhydrous DMF (2.4 L, 8 vol.) was added, and stirring was begun. The solution was cooled to 13° C., and K₃PO₄ (1.13 kg, 5.33 mol, 2 eq) was added. 2,4-difluoropyridine (613.9 g, 5.33 mol, 2 eq) was added to the reactor, and the reaction was stirred until complete. The reaction mixture was filtered, and the filtrate was transferred slowly into a reactor containing water (6 L, 20 vol.). The resulting slurry was stirred for 1 h. The slurry was then filtered, and the wet cake was washed with water and dried in a vacuum oven at 60° C. Crude 2-fluoro-4-(3-nitro-1H-pyrazol-1-yl)pyridine was isolated in 89% yield as an off white solid.

2-fluoro-4-(3-nitro-1H-pyrazol-1-yl)pyridine was separated from 2,4-bis(3-nitro-1H-pyrazol-1-yl)pyridine (formed as a side product) by recrystallization. A reactor was charged with crude 2-fluoro-4-(3-nitro-1H-pyrazol-1-yl)pyridine (944.1 g), dichloromethane (8.5 L, 9 vol.), and methanol (19.8 L, 21 vol.), and the agitation was set to 150 rpm. The slurry was stirred at 39° C. for about 4 h, and then the jacket temperature was ramped down to 20° C., and stirring was continued for 30 minutes. The reaction mixture was filtered, and the wet cake was rinsed with methanol (0.5 L, 0.6 vol.). The filtrate was concentrated, and the resulting slurry was filtered. The wet cake was rinsed with methanol and then dried in a vacuum oven at 50-55° C. with nitrogen bleed. 2-fluoro-4-(3-nitro-1H-pyrazol-1-yl)pyridine was isolated in 75% yield (708 g) as a white solid.

### Step 2: 1-(2-fluoropyridin-4-yl)-1H-pyrazol-3-amine

2-fluoro-4-(3-nitro-1H-pyrazol-1-yl)pyridine (808 g, 3.88 mol, 1 eq), 3% platinum on carbon catalyst (66% wet) (37.9 g, 1.94 mol, 0.0005 eq), and 2:1 tetrahydrofuran:methanol (13.6 L, 17 vol.) were loaded into a jacketed hydrogenator. The hydrogenator was purged with nitrogen and was then purged with hydrogen. The hydrogen was charged to a pressure of 3.0 bar, and the jacket temperature was ramped to 50° C. over 1 hour. Stirring was maintained between about 800 and 1,000 RPM. The batch was stirred until complete conversion was achieved (10 hours). The batch was cooled to 30° C. and filtered over a Celite pad to remove the catalyst. The filter cake was washed with 2:1 tetrahydrofuran:methanol (1.76 L, 2 vol.), the tetrahydrofuran/methanol mother liquors were stripped to dry solid, and two chases of isopropyl alcohol (each 5 volumes) were performed to remove as much tetrahydrofuran as possible. The solids were then taken up in 8 volumes of isopropyl alcohol (6.5 L) and heated to 80° C. Once temperature was reached, 4 volumes of water (3.2 L) were added over 1 hour to afford a clear, yellow solution. The solution was cooled to 70° C. and was seeded with crystals of 1-(2-fluoropyridin-4-yl)-1H-pyrazol-3-amine (0.05 wt %, 4 g). Crystals were allowed to grow as the batch was cooled from 70° C. to 60° C. over 1 hour, and then another 12 volumes of water (9.7 L) were added over two hours. Once the water addition was complete, the batch was cooled from 60° C. to 20° C. over 5 hours and was then filtered and washed with 2 volumes of 2:1 water:isopropyl alcohol (2.4 mL). The solids were dried in an oven at 45° C. with a nitrogen sweep until a constant weight was obtained. 1-(2-fluoropyridin-4-yl)-1H-pyrazol-3-amine was obtained in 88% yield.

### Example: 1-(pyridazin-3-yl)-1H-pyrazol-3-amine

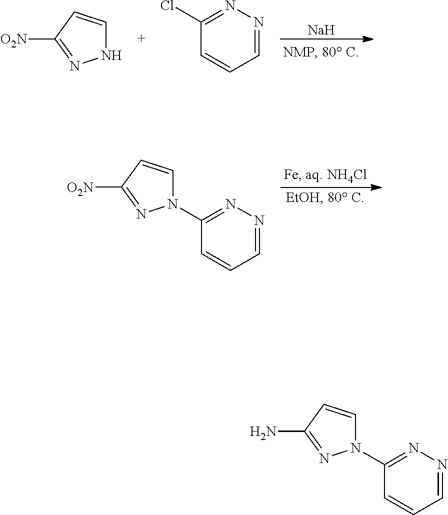

### Step 1: 3-(3-nitro-1H-pyrazol-1-yl)pyridazine

To a 0° C. solution of 3-nitro-1H-pyrazole (1.5 g, 13.27 mmol) in NMP (1.2 mL) was added NaH (1.2 g of 60% w/w, 30.00 mmol). After 20 minutes, the mixture was warmed to room temperature and stirred a further 60 min. The mixture was re-cooled to 0° C. and 3-chloropyridazine (hydrochloride salt; 2.0 g, 13.25 mmol) was added. The resultant mixture was heated to 80° C. and stirred for 16 h. The reaction mixture was poured over ice, resulting in precipitation of a solid. The product was collected by vacuum filtration, and the solids air-dried to provide 3-(3-nitro-1H-pyrazol-1-yl)pyridazine (1.51 g, 58% yield) as a beige solid. 1H NMR (400 MHz, DMSO-d₆) δ 9.38 (dd, J=4.8, 1.4 Hz, 1H), 9.11 (d, J=2.8 Hz, 1H), 8.32 (dd, J=8.9, 1.4 Hz, 1H), 8.03 (dd, J=8.9, 4.8 Hz, 1H), 7.43 (d, J=2.8 Hz, 1H) ppm. ESI-MS m/z calc. 191.04, found 192.04 (M+1).

### Step 2: 1-(pyridazin-3-yl)-1H-pyrazol-3-amine

3-(3-nitropyrazol-1-yl)pyridazine (1.5 g, 7.69 mmol) was dissolved in ethanol (40.0 mL) at room temperature. To the resultant solution was added aqueous ammonium chloride (7.0 mL of 7 M, 49.00 mmol) and iron (2.0 g, 35.81 mmol). The resultant mixture was stirred 4 h at 80° C. under nitrogen. The reaction mixture was filtered through Celite, and the filter pad was washed with ethanol and ethyl acetate. The combined filtrate was concentrated to a white solid. The solid was dissolved in dichloromethane and dried (Na₂SO₄). After filtration, the solvent was evaporated to provide 1-(pyridazin-3-yl)-1H-pyrazol-3-amine (1.3 g, 52% yield) as a white solid. ESI-MS m/z calc. 161.07, found 162.10 (M+1).

### Example: 1-(pyrimidin-2-yl)-1H-pyrazol-3-amine

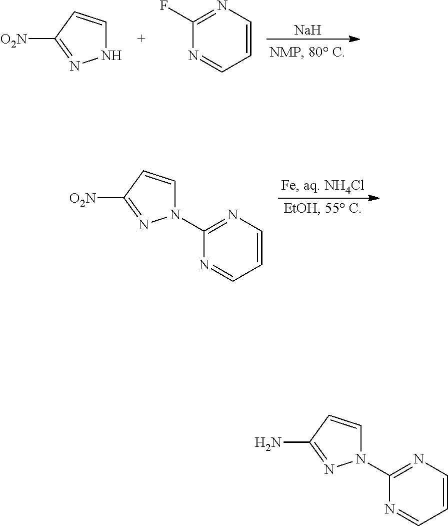

### Step 1: 2-(3-nitro-1H-pyrazol-1-yl)pyrimidine

To a 0° C. solution of 3-nitro-1H-pyrazole (1.0 g, 8.84 mmol) in NMP (10.0 mL) was added NaH (425 mg of 60% w/w, 10.63 mmol). After 20 minutes, the mixture was warmed to room temperature and stirred a further 60 minutes. The mixture was re-cooled to 0° C. and 2-fluoropyrimidine (1.0 g, 10.20 mmol) was added. The resultant mixture was heated to 80° C. for 16 h. The reaction mixture was poured over ice, resulting in precipitation of a solid. The product was collected by vacuum filtration, and the solids air-dried to provide 2-(3-nitro-1H-pyrazol-1-yl)pyrimidine (1.66 g, 96% yield). 1H NMR (400 MHz, DMSO-d₆) δ 9.00 (d, J=4.8 Hz, 2H), 8.91 (d, J=2.9 Hz, 1H), 7.68 (t, J=4.9 Hz, 1H), 7.35 (d, J=2.8 Hz, 1H) ppm. ESI-MS m/z calc. 191.04, found 191.96 (M+1).

### Step 2: 1-(pyrimidin-2-yl)-1H-pyrazol-3-amine

2-(3-nitropyrazol-1-yl)pyrimidine (1.65 g, 8.20 mmol) was dissolved in ethanol (10.0 mL) at room temperature. To the resultant solution was added aqueous ammonium chloride (8.0 mL of 7 M, 56.00 mmol) and iron (2.1 g, 37.60 mmol). The resultant mixture was stirred 16 h at 55° C. under nitrogen. The reaction mixture was filtered through Celite, and the filter pad was washed with ethanol and ethyl acetate. The filtrate was concentrated to a white solid. The solid was dissolved in dichloromethane and dried (Na₂SO₄). After filtration, the solvent was evaporated to provide 1-(pyrimidin-2-yl)-1H-pyrazol-3-amine (138 mg, 10% yield) as a yellow waxy solid. 1H NMR (300 MHz, DMSO-d₆) δ 8.69 (d, J=4.8 Hz, 2H), 8.30 (d, J=2.7 Hz, 1H), 7.22 (t, J=4.8 Hz, 1H), 5.87 (d, J=2.7 Hz, 1H), 5.30 (s, 2H) ppm. ESI-MS m/z calc. 161.07, found 162.12 (M+1).

### Example 1.2. Acid Intermediates

All carboxylic acids were purchased commercially, with the exception of those shown below (see Scheme Acid-1).

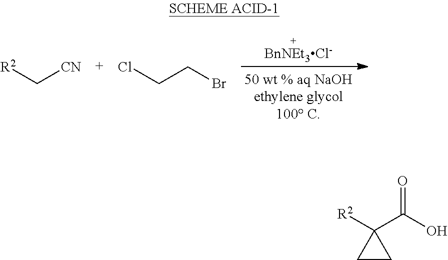

Scheme Acid-1, shown above, provides a general synthetic route for the preparation of 1-aryl-cyclopropane-1-carboxylic acids. Carboxylic acid intermediates were synthesized using appropriate choice of aryl acetonitrile following the procedure outlined below for 1-(4-chloro-2-fluorophenyl)cyclopropane-1-carboxylic acid.

### Example: 1-(4-chloro-2-fluorophenyl)cyclopropane-1-carboxylic acid

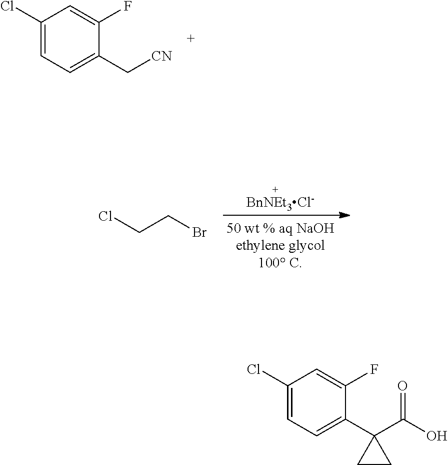

To a solution of benzyl(triethyl)ammonium chloride (27 mg, 0.12 mmol) in ethylene glycol (8.0 mL) was added 1-bromo-2-chloro-ethane (880 μL, 10.61 mmol), 2-(4-chloro-2-fluorophenyl)acetonitrile (1.0 g, 5.90 mmol), and 50% w/v aqueous NaOH (3.3 mL, 41.28 mmol). The resultant reaction mixture was stirred at 100° C. for 18 h. The reaction mixture was cooled to room temperature and diluted with water (100 mL). The aqueous layer was extracted with ethyl acetate (2×100 mL), and the organic fractions were discarded. The aqueous fraction was acidified to pH 1 by addition of 6N HCl and extracted with ethyl acetate (2×100 mL). The combined organic fractions were washed with water (100 mL) and brine (100 mL), dried (Na₂SO₄), filtered, and concentrated to provide crude 1-(4-chloro-2-fluorophenyl)cyclopropane-1-carboxylic acid (1.08 g, 85% yield) which was used without further purification. ¹H NMR (300 MHz, DMSO-d₆) δ 12.50 (s, 1H), 7.45-7.08 (m, 3H), 1.48 (m, 2H), 1.16 (m, 2H) ppm.

### Example: 1-(2,5-difluorophenyl)cyclopropane-1-carboxylic acid

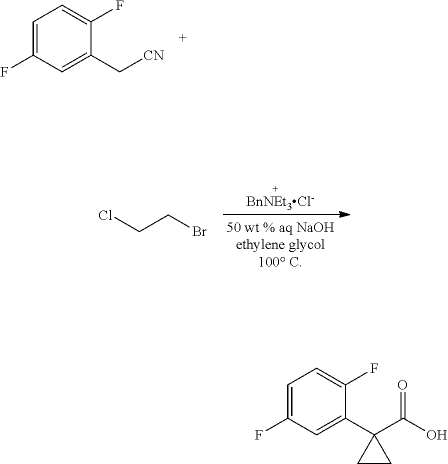

Prepared according to the procedure described for 1-(4-chloro-2-fluorophenyl)cyclopropane-1-carboxylic acid using 2-(2,5-difluorophenyl)acetonitrile as a starting material in place of 2-(4-chloro-2-fluorophenyl)acetonitrile. Product obtained in 81% yield. ¹H NMR (300 MHz, DMSO-d₆) δ 12.50 (s, 1H), 7.25-7.11 (m, 3H), 1.47 (m, 2H), 1.20 (m, 2H) ppm.

### Example: 1-(5-chloro-2-fluorophenyl)cyclopropane-1-carboxylic acid

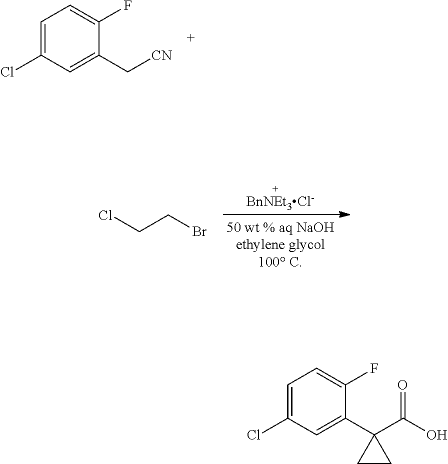

Prepared according to the procedure described for 1-(4-chloro-2-fluorophenyl)cyclopropane-1-carboxylic acid using 2-(5-chloro-2-fluorophenyl)acetonitrile as a starting material in place of 2-(4-chloro-2-fluorophenyl)acetonitrile. Product obtained in 78% yield. ¹H NMR (300 MHz, DMSO-d₆) δ 12.52 (s, 1H), 7.39 (m, 2H), 7.22 (m, 1H), 1.47 (m, 2H), 1.21 (m, 2H) ppm.

### Example: 1-(2,6-difluorophenyl)cyclopropane-1-carboxylic acid

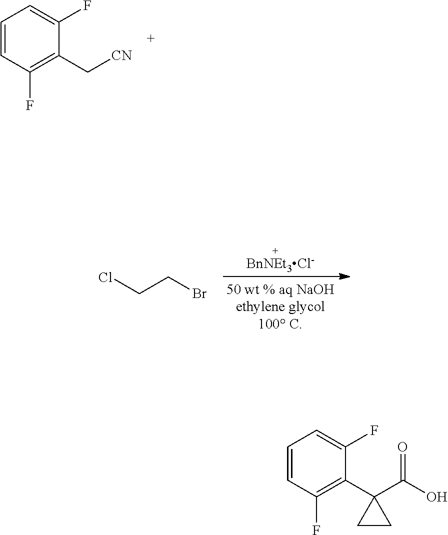

Prepared according to the procedure described for 1-(4-chloro-2-fluorophenyl)cyclopropane-1-carboxylic acid using 2-(2,6-difluorophenyl)acetonitrile as a starting material in place of 2-(4-chloro-2-fluorophenyl)acetonitrile. Product obtained in 72% yield. ¹H NMR (300 MHz, DMSO-d₆) δ 12.61 (s, 1H), 7.38 (m, 1H), 7.21-6.96 (m, 2H), 1.57 (m, 2H), 1.19 (m, 2H) ppm.

### Example: 1-(2,3-difluorophenyl)cyclopropane-1-carboxylic acid

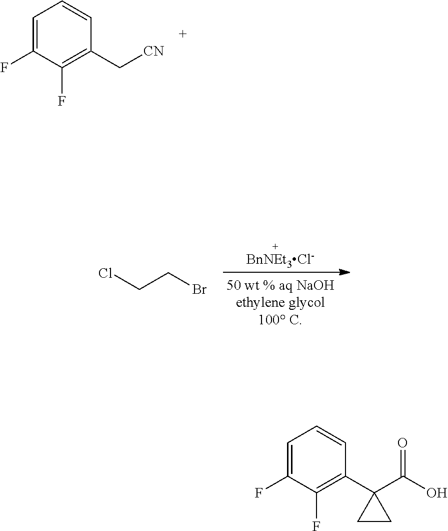

Prepared according to the procedure described for 1-(4-chloro-2-fluorophenyl)cyclopropane-1-carboxylic acid using 2-(2,3-difluorophenyl)acetonitrile as a starting material. Product obtained in 86% yield. ¹H NMR (300 MHz, DMSO-d₆) δ 12.53 (s, 1H), 7.48-7.26 (m, 1H), 7.26-7.01 (m, 2H), 1.50 (m, 2H), 1.21 (m, 2H) ppm.

### Example: 1-(3,5-difluorophenyl)cyclopropane-1-carboxylic acid

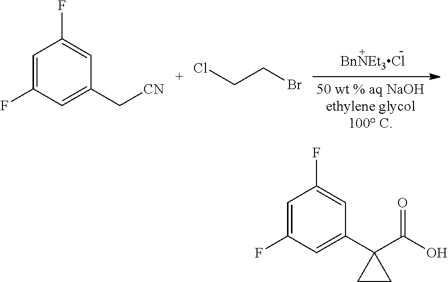

Prepared according to the procedure described for 1-(4-chloro-2-fluorophenyl)cyclopropane-1-carboxylic acid using 2-(3,5-difluorophenyl)acetonitrile as a starting material in place of 2-(4-chloro-2-fluorophenyl)acetonitrile. Product obtained in 81% yield. ¹H NMR (300 MHz, DMSO-d₆) δ 12.48 (s, 1H), 7.09 (m, 3H), 1.44 (m, 2H), 1.32-1.10 (m, 2H) ppm.

### Example: 1-(2-chloro-6-fluoro-3-methylphenyl)cyclopropane-1-carboxylic acid

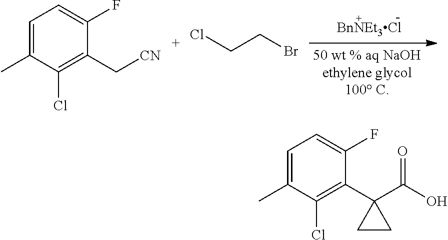

Prepared according to the procedure described for 1-(4-chloro-2-fluorophenyl)cyclopropane-1-carboxylic acid using 2-(2-chloro-6-fluoro-3-methylphenyl)acetonitrile as a starting material in place of 2-(4-chloro-2-fluorophenyl)acetonitrile. Product obtained in 79% yield. ¹H NMR (300 MHz, DMSO-d₆) δ 12.52 (s, 1H), 7.33 (m, 1H), 7.24-7.02 (m, 1H), 2.31 (s, 3H), 1.65 (s, 2H), 1.15 (s, 2H) ppm.

### Example: 1-(2-chloro-6-fluorophenyl)cyclopropane-1-carboxylic acid

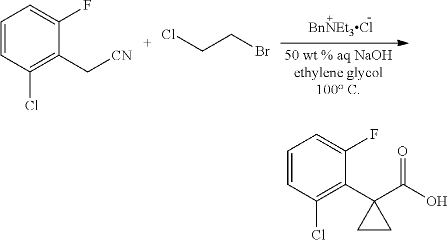

Prepared according to the procedure described for 1-(4-chloro-2-fluorophenyl)cyclopropane-1-carboxylic acid using 2-(2-chloro-6-fluorophenyl)acetonitrile as a starting material in place of 2-(4-chloro-2-fluorophenyl)acetonitrile. Product obtained in 84% yield. ¹H NMR (300 MHz, DMSO-d₆) δ 12.55 (s, 1H), 7.46-7.14 (m, 3H), 1.65 (m, 2H), 1.21 (m, 2H) ppm.

### Example: 1-(2-chloro-6-fluorophenyl)cyclopropane-1-carboxylic acid

Purchased commercially or prepared according to the procedure described for 1-(4-chloro-2-fluorophenyl)cyclopropane-1-carboxylic acid using 2-(2-fluorophenyl)acetonitrile as a starting material in place of 2-(4-chloro-2-fluorophenyl)acetonitrile. Product obtained in 96% yield. ¹H NMR (400 MHz, CDCl₃) δ 12.04 (s, 1H), 7.36-7.21 (m, 2H), 7.17-6.99 (m, 2H), 1.75 (q, J=4.1 Hz, 2H), 1.29 (q, J=4.2 Hz, 2H) ppm. ESI-MS m/z calc. 180.05865, found 181.15 (M+1).

### Example: 1-(2-fluoro-5-methoxyphenyl)cyclopropane-1-carboxylic acid

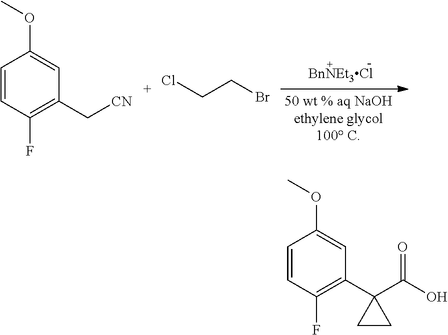

Prepared according to the procedure described for 1-(4-chloro-2-fluorophenyl)cyclopropane-1-carboxylic acid using 2-(2-fluoro-5-methoxyphenyl)acetonitrile as a starting material in place of 2-(4-chloro-2-fluorophenyl)acetonitrile. Product obtained in 94% yield. ¹H NMR (300 MHz, DMSO-d₆) δ 12.37 (s, 1H), 7.09-7.01 (m, 1H), 6.87-6.80 (m, 2H), 1.46 (m, 2H), 1.16 (m, 2H) ppm.

### Example: 1-(2-fluorophenyl)cyclopropane-1-carboxylic-2,2,3,3-d₄ acid (for Compound 276)

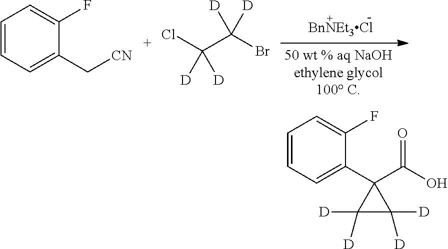 [SMILES: N#CCc1ccccc1F ; [2H]C([2H])(Cl)C([2H])([2H])Br ; [2H]C1([2H])C([2H])([2H])C1(C(=O)O)c1ccccc1F]

Benzyl(triethyl)ammonium chloride (47 mg, 0.21 mmol), 1-bromo-2-chloroethane-1,1,2,2-d₄ (2.05 g, 13.90 mmol), and 2-fluorophenyl-acetonitrile (1.27 g, 9.40 mmol) were combined. 50% w/v aqueous NaOH (6.0 mL) was added dropwise over 5 minutes with stirring. The resultant reaction mixture was heated to 46° C. for 24 h. Disappearance of the starting material was confirmed by HPLC. Ethylene glycol (5.0 mL) was added, and the mixture was stirred 24 h at 100° C. The reaction mixture was cooled to room temperature and partitioned between water and diethyl ether. The layers were separated, and the aqueous layer was further extracted with diethyl ether. The ether fractions were discarded. The aqueous fraction was acidified to pH 1 by addition of concentrated HCl (8.0 mL) and extracted twice with diethyl ether. The combined organics were washed with water and brine (100 mL), dried (Na₂SO₄), filtered, and concentrated to provide crude 1-(2-fluorophenyl)cyclopropane-1-carboxylic-2,2,3,3-d₄ acid (1.08 g, 85% yield) which was used without further purification.

### Example: 2-ethyl-2-methyl-1-phenylcyclopropane-1-carboxylic acid

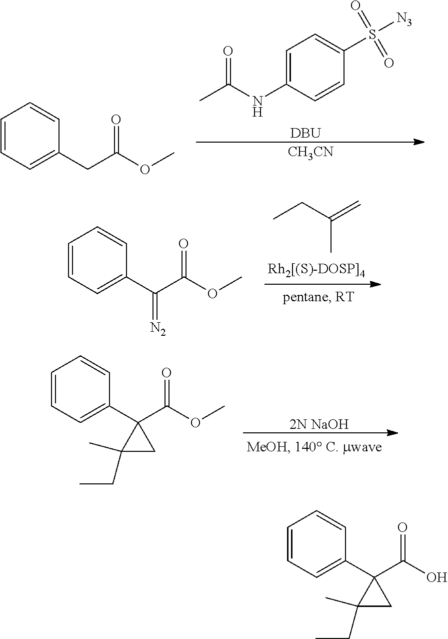

### Step 1: Methyl 2-diazo-2-phenylacetate

To a mixture of methyl 2-phenylacetate (5.0 g, 33.3 mmol) and 4-acetamidobenzenesulfonyl azide (8.8 g, 36.7 mmol) in acetonitrile (20 mL) was added DBU (6.1 g, 40.0 mmol). The reaction mixture was stirred at room temperature for 16 h then partitioned between water and ethyl acetate. The layers were separated, and the aqueous further extracted with ethyl acetate. The combined organics were washed with brine, dried (MgSO₄), filtered, and concentrated. The crude material was purified by silica gel chromatography (isocratic 10% ethyl acetate/heptane) to provide methyl 2-diazo-2-phenylacetate (4.8 g, 89% yield). ¹H NMR (400 MHz, CDCl₃) δ 3.87 (s, 3H), 7.17-7.20 (m, 1H), 7.36-7.40 (m, 2H), 7.47-7.49 (m, 2H) ppm.

### Step 2: Methyl 2-ethyl-2-methyl-1-phenylcyclopropane-1-carboxylate

2-Methylbut-1-ene (2.78 g, 39.6 mmol) and Rh₂[(R)-DOSP]₄ were combined in pentane (450 mL) under nitrogen atmosphere. Methyl 2-diazo-2-phenyl-acetate (3.49 g, 19.8 mmol) was then added dropwise as a solution in pentane (60 mL). The resultant mixture was stirred for 1 h, and the solvent was subsequently removed in vacuo. The crude residue was purified by silica gel chromatography (linear gradient 0-10% ethyl acetate/heptane) to provide methyl 2-ethyl-2-methyl-1-phenylcyclopropane-1-carboxylate (2.8 g, 65% yield) as a scalemic mixture. ESI-MS m/z calc. 218.13, found 219.45 (M+1).

### Step 3: 2-ethyl-2-methyl-1-phenylcyclopropane-1-carboxylic acid

Methyl 2-ethyl-2-methyl-1-phenyl-cyclopropanecarboxylate (1.1 g, 5.04 mmol) was dissolved in methanol (7.0 mL) and 2N NaOH (5.0 mL). The resultant mixture was heated for 15 min at 140° C. in microwave. The mixture was acidified to pH 4 with 1N HCl and extracted three times with ethyl acetate. The combined organics were dried (Na₂SO₄), filtered, and concentrated to provide 2-ethyl-2-methyl-1-phenylcyclopropane-1-carboxylic acid (0.98 g; 95% yield, white solid) as a scalemic mixture that was used without further purification. ESI-MS m/z calc. 204.12, found 205.46 (M+1).

### Example: 1-phenylspiro[2.4]heptane-1-carboxylic acid; (S)-1-phenylspiro[2.4]heptane-1-carboxylic acid; and (R)-1-phenylspiro[2.4]heptane-1-carboxylic acid

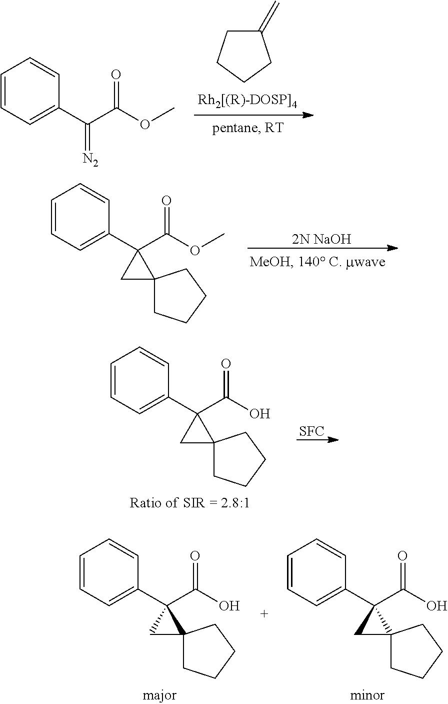

### Step 1: methyl 1-phenylspiro[2.4]heptane-1-carboxylate

To a room temperature solution of methyl 2-diazo-2-phenyl-acetate (5.0 g, 28.38 mmol) in pentane (150 mL) under nitrogen was added Rh₂[(R)-DOSP]₄ (250 mg, 0.005 mmol). To the resultant mixture was added methylenecyclopentane (7.0 g, 85.14 mmol) dropwise as a solution in pentane (20 mL). The reaction mixture was stirred for 1 h then the solvent was removed in vacuo. The crude residue was purified by silica gel chromatography (linear gradient 0-10% ethyl acetate/heptane) to provide methyl 1-phenylspiro[2.4]heptane-1-carboxylate (5.0 g, 77% yield) as a scalemic mixture. The absolute stereochemistry of the major enantiomer was presumed to be (5) based on literature precedent (Org. Lett. 2008, 10, 573), and this stereochemical preference was confirmed by X-ray crystallography after Step 3 (vide infra). ¹H NMR (300 MHz, CDCl₃) δ 7.58-7.10 (m, 5H), 3.64 (s, 3H), 1.89 (d, J=4.5 Hz, 1H), 1.86-1.55 (m, 6H), 1.43 (dt, J=13.0, 7.2 Hz, 1H), 1.35 (d, J=4.5 Hz, 1H), 1.00 (dt, J=13.2, 6.7 Hz, 1H) ppm. ESI-MS m/z calc. 230.13, found 231.47 (M+1).

### Step 2: 1-phenylspiro[2.4]heptane-1-carboxylic acid

Methyl 1-phenylspiro[2.4]heptane-1-carboxylate (5.0 g, 21.71 mmol) was dissolved in methanol (30.0 mL) and 2N NaOH (21.7 mL). The resultant mixture was heated for 15 min at 140° C. in microwave. The solvent was removed in vacuo, and the crude residue was partitioned between 1N HCl and dichloromethane. The layers were separated, and the aqueous further extracted with dichloromethane. The combined organics were washed with water, dried (Na₂SO₄), filtered, and concentrated to provide 1-phenylspiro[2.4]heptane-1-carboxylic acid (4.0 g, 85% yield, white solid) as a scalemic mixture. ¹H NMR (300 MHz, CDCl₃) δ 11.65 (s, 1H), 7.64-6.98 (m, 5H), 2.05-1.59 (m, 7H), 1.55-1.39 (m, 2H), 1.02 (dt, J=13.3, 6.6 Hz, 1H) ppm. ESI-MS m/z calc. 216.12, found 217.47 (M+1).

### Step 3: (S)-1-phenylspiro[2.4]heptane-1-carboxylic acid and (R)-1-phenylspiro[2.4]heptane-1-carboxylic acid

The enantiomeric mixture from the hydrolysis Step 2 was purified by SFC using 20×250 mm OJ-H column with isocratic 40% methanol (0.2% diethylamine), 60% CO₂ as mobile phase. The ratio of SIR enantiomers was determined to be 2.8:1. The absolute stereochemistry of the major enantiomer was confirmed by X-ray crystallography on the N-(1-(4-bromophenyl)ethyl)-1-phenylspiro[2.4]heptane-1-carboxamide derivative prepared from (R)-1-(4-bromophenyl)ethan-1-amine.

### Example: 1-Phenylspiro[2.3]hexane-1-carboxylic acid

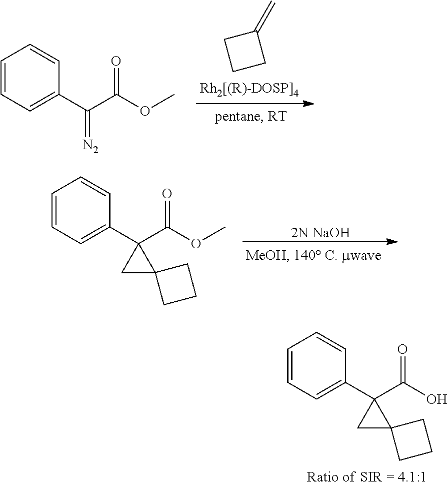

### Step 1: Methyl 1-phenylspiro[2.3]hexane-1-carboxylate

To a room temperature solution of methyl 2-diazo-2-phenyl-acetate (1.12 g, 6.36 mmol) in pentane (150 mL) under nitrogen was added Rh₂[(R)-DOSP]₄ (56 mg, 0.03 mmol). To the resultant mixture was added methylene cyclobutane (1.3 g, 19.08 mmol) dropwise as a solution in pentane (20 mL). The reaction mixture was stirred for 1 h then the solvent was removed in vacuo. The crude residue was purified by silica gel chromatography (linear gradient 0-10% ethyl acetate/heptane) to provide methyl 1-phenylspiro[2.3]hexane-1-carboxylate (1.33 g, 97% yield) as a scalemic mixture. ESI-MS m/z calc. 216.12, found 217.43 (M+1).

### Step 2: 1-Phenylspiro[2.3]hexane-1-carboxylic acid

Methyl 1-phenylspiro[2.3]hexane-1-carboxylate (150 mg, 0.69 mmol) was dissolved in methanol (3.0 mL) and 2N NaOH (1.0 mL). The resultant mixture was heated for 15 min at 140° C. in microwave. The mixture was acidified to pH 4 with 1N HCl and extracted three times with ethyl acetate. The combined organics were dried (Na₂SO₄), filtered, and concentrated to provide 1-phenylspiro[2.3]hexane-1-carboxylic acid (0.98 g; 95% yield) as a white solid. Chiral analytical SFC showed that the product is a 4.1:1 mixture of enantiomers. The absolute stereochemistry of the major enantiomer was presumed to be (S), consistent with literature precedent (Org. Lett. 2008, 10, 573) and similar to the cyclopropanation transformation described above for methyl 1-phenylspiro[2.4]heptane-1-carboxylate. The scalemic mixture was used without further purification. ESI-MS m/z calc. 204.12, found 205.46 (M+1).

### Example: 1-(3-fluoropyridin-2-yl)cyclopropane-1-carboxylic acid

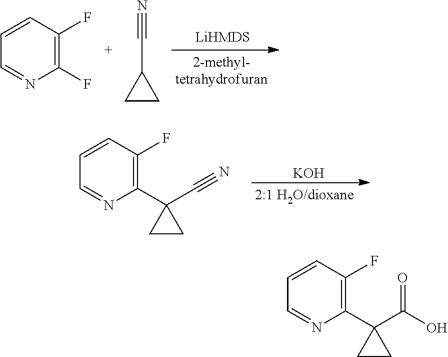

### Step 1: 1-(3-fluoropyridin-2-yl)cyclopropane-1-carbonitrile

To a solution of cyclopropanecarbonitrile (49.0 mL, 665.4 mmol) in 2-methyltetrahydrofuran (600 mL) at 0° C. (ice-water bath) was added lithium bis(trimethylsilyl)amide (650 mL of 1M solution in hexanes, 650 mmol) over 25 minutes. After 10 minutes, 2,3-difluoropyridine (19.76 mL, 217.2 mmol) was added. The cooling bath was removed and reaction was warmed to room temperature and stirred for 3 h. The reaction was quenched by addition of saturated aqueous ammonium chloride (20 mL). The resultant mixture was partitioned between water and ethyl acetate. The organics were collected and washed with saturated aqueous sodium bicarbonate and brine, dried (MgSO₄), filtered, and concentrated. The crude residue was purified by silica gel chromatography (linear gradient of 0-70% EtOAc) to 1-(3-fluoro-2-pyridyl)cyclopropanecarbonitrile (25.3 g, 72%) as a yellow oil. 1H NMR (400 MHz, Chloroform-d) δ 8.16 (dt, J=4.6, 1.4 Hz, 1H), 7.29 (ddd, J=10.2, 8.3, 1.4 Hz, 1H), 7.15-7.07 (m, 1H), 1.70-1.63 (m, 2H), 1.63-1.56 (m, 2H) ppm. ESI-MS m/z calc. 162.06, found 163.08 (M+1).

### Step 2: 1-(3-fluoropyridin-2-yl)cyclopropane-1-carboxylic acid

To a solution potassium hydroxide 22.7 g, 343.9 mmol) in water (200 mL) was added a solution of 1-(3-fluoro-2-pyridyl)cyclopropanecarbonitrile (25.3 g, 156.0 mmol) in dioxane (100 mL). The resultant mixture was heated to 90° C. for 18 h. The solution was cooled to room temperature, then aqueous 6 N HCl (2.5 mL) was added until the pH 3 was reached. The mixture was cooled in an ice-water bath with stirring to give a suspension of white precipitate. The precipitate was collected via filtration, washing with water (2×2 mL). The filter cake was dried under vacuum at 70° C. to furnish 1-(3-fluoro-2-pyridyl)cyclopropanecarboxylic acid (25.7 g, 91%) as a white powder. 1H NMR (400 MHz, DMSO-d₆) δ 12.53 (s, 1H), 8.31 (dt, J=4.7, 1.5 Hz, 1H), 7.67 (ddd, J=10.0, 8.3, 1.4 Hz, 1H), 7.40 (dt, J=8.3, 4.4 Hz, 1H), 1.49 (q, J=4.0 Hz, 2H), 1.38-1.32 (m, 2H) ppm. ESI-MS m/z calc. 181.05391, found 182.07 (M+1)+; Retention time: 0.53 minutes.

### Example: 1-(5-chloro-3-fluoropyridin-2-yl)cyclopropane-1-carboxylic acid

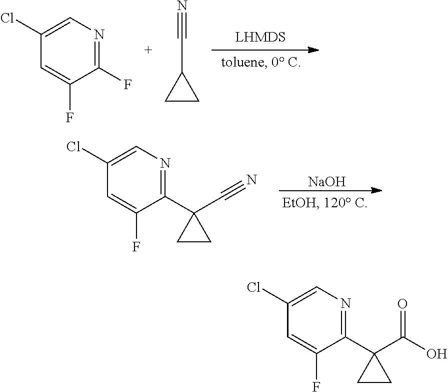

### Step 1: 1-(5-chloro-3-fluoropyridin-2-yl)cyclopropane-1-carbonitrile

A solution of cyclopropanecarbonitrile (650 μL, 8.826 mmol) in toluene (5.0 mL) was cooled to 0° C. Lithium bis(trimethylsilyl)amide (17 mL of 0.5 M toluene solution, 8.500 mmol) was added, and the resultant reaction mixture was warmed to room temperature and stirred for 30 minutes. The above solution was added to 5-chloro-2,3-difluoro-pyridine (1.3 g, 8.694 mmol) in toluene (5 mL) at room temperature, and stirring was continued overnight. The reaction was mixture was partitioned between saturated aqueous NaHCO3 and EtOAc. The organics were collected, washed with brine and water, dried (Na2SO4), filtered, and concentrated. The crude residue was purified by silica gel chromatography (linear gradient of 0-100% ethyl acetate/heptane to provide 1-(5-chloro-3-fluoro-2-pyridyl)cyclopropanecarbonitrile (62 mg, 3%) ESI-MS m/z calc. 196.02, found 197.04 (M+1).

### Step 2: 1-(5-chloro-3-fluoropyridin-2-yl)cyclopropane-1-carboxylic acid

1-(5-chloro-3-fluoro-2-pyridyl)cyclopropanecarbonitrile (60 mg, 0.220 mmol) was suspended in NaOH (1.0 mL of 6 M aqueous solution, 6.000 mmol) and EtOH (0.5 mL). The resultant mixture was stirred in a sealed vial at 120° C. overnight. The mixture was cooled to room temperature, and 6M HCl (1.0 mL, 6.000 mmol) was added. The solution was purified by reverse phase C18 chromatography (100 g C18 column, eluting with 10-100% ACN in water with 0.1% TFA) to provide 1-(5-chloro-3-fluoro-2-pyridyl)cyclopropanecarboxylic acid (TFA salt) (11.9 mg, 16%). ESI-MS m/z calc. 215.015, found 216.04 (M+1).

### Example: 1-(3-fluoro-5-methylpyridin-2-yl)cyclopropane-1-carboxylic acid

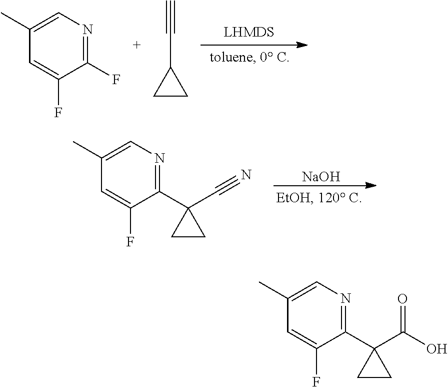

Prepared by analogous procedure to the one described above for 1-(5-chloro-3-fluoropyridin-2-yl)cyclopropane-1-carboxylic acid. Product obtained in 0.3% yield. ESI-MS m/z calc. 195.07, found 196.05 (M+1).

### Example: 1-(3-fluoropyridin-2-yl)spiro[2.2]pentane-1-carboxylic acid

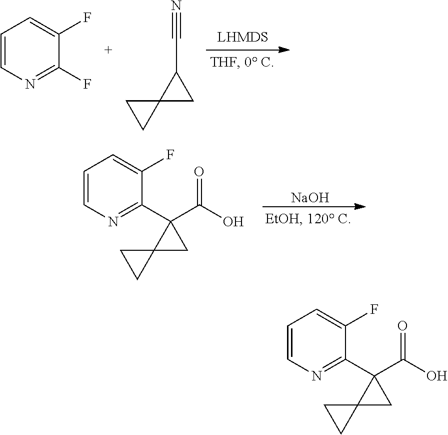

Prepared by procedure analogous to the one described above for 1-(5-chloro-3-fluoropyridin-2-yl)cyclopropane-1-carboxylic acid except using THF as solvent in the Step 1 rather than toluene. Product obtained in 53% yield (2 steps). 1H NMR (400 MHz, DMSO-d6) δ 12.42 (s, 1H), 8.34 (dt, J=4.7, 1.6 Hz, 1H), 7.66 (ddd, J=9.9, 8.3, 1.4 Hz, 1H), 7.40 (dt, J=8.6, 4.4 Hz, 1H), 1.97 (dd, J=34.3, 3.9 Hz, 2H), 1.26-0.95 (m, 2H), 0.78 (ddt, J=25.7, 9.8, 5.1 Hz, 2H) ppm. ESI-MS m/z calc. 207.07, found 208.07 (M+1).

### Example: 1-(3-fluoro-2-pyridyl)-2-methyl-cyclopropanecarboxylic acid

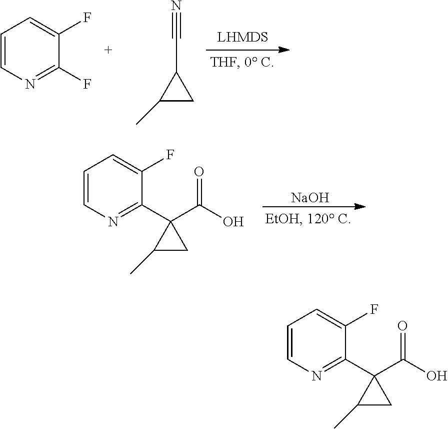

Prepared by procedure analogous to the one described above for 1-(5-chloro-3-fluoropyridin-2-yl)cyclopropane-1-carboxylic acid except using THF as solvent in the Step 1 rather than toluene. Product obtained in 83% yield (2 steps). ESI-MS m/z calc. 195.07, found 196.05 (M+1).

### Example: 1-(3-fluoropyridin-2-yl)-2,2-dimethylcyclopropane-1-carboxylic acid

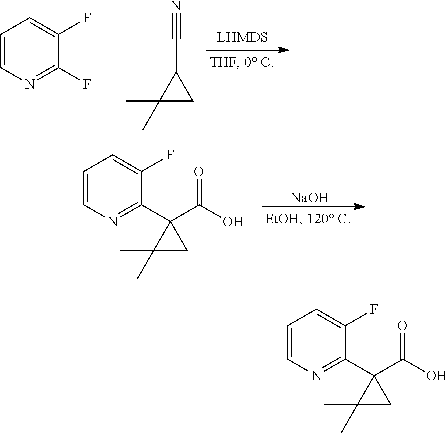

Prepared by analogous procedure to the one described above for 1-(5-chloro-3-fluoropyridin-2-yl)cyclopropane-1-carboxylic acid except using THF as solvent in the first step rather than toluene (gives yield improvement). Product obtained in 53% yield (2 steps). ESI-MS m/z calc. 190.09, found 191.1 (M+1).

### Example 1.3. Compounds Prepared Using Amide Bond Formation as Final Step

Amide bond formation is described below in Scheme Amide-1 (Methods A-AE).

### Scheme Amide-1. Preparation of Compounds in Table A

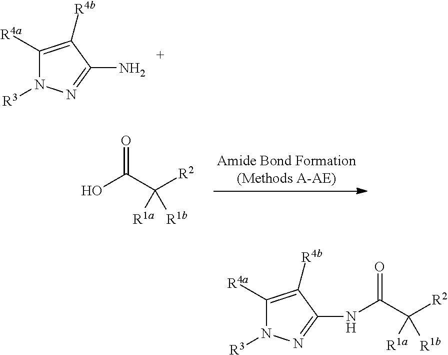

Scheme Amide-1 provides a general synthetic route for the preparation of compounds listed in Table A. Using the appropriate selection of carboxylic acid and amine, compounds within Table A were synthesized according to one of the following amide coupling procedures, Methods A-AE. A representative example of each method is provided, and the coupling method used to prepare each compound as well as yield and characterization information is provided in Table A.

### Method A

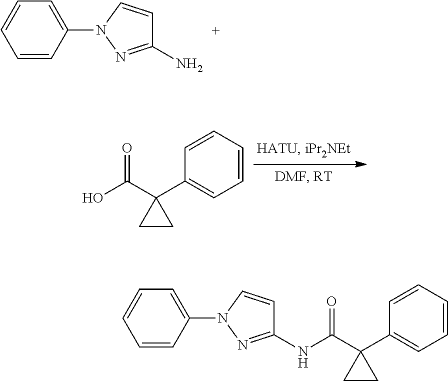 [SMILES: Nc1ccn(-c2ccccc2)n1 ; O=C(O)C1(c2ccccc2)CC1 ; O=C(Nc1ccn(-c2ccccc2)n1)C1(c2ccccc2)CC1]

### 1-phenyl-N-(1-phenyl-1H-pyrazol-3-yl)cyclopropane-1-carboxamide (Compound 2)

To a solution of 1-phenylpyrazol-3-amine (50 mg, 0.31 mmol, 1.0 eq) in DMF (2.0 mL) was added 1-phenylcyclopropane-1-carboxylic acid (101.8 mg, 0.63 mmol, 2.0 eq), iPr₂NEt (165 μL, 0.94 mmol, 3.0 eq), and HATU (143 mg, 0.38 mmol, 1.2 eq). The resultant mixture was stirred at room temperature for 3 h. The reaction mixture was filtered, and the filtrate was concentrated. The crude residue was purified by C18 preparatory HPLC (acetonitrile/water with HCl modifier) to provide 1-phenyl-N-(1-phenyl-1H-pyrazol-3-yl)cyclopropane-1-carboxamide (56.2 mg, 59% yield).

### Method B

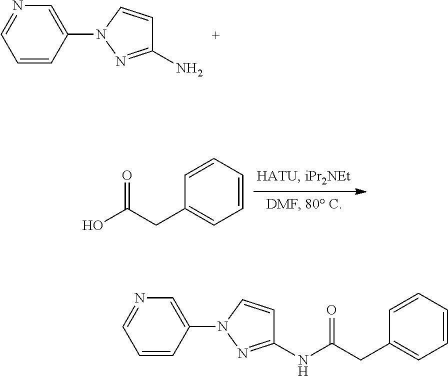 [SMILES: Nc1ccn(-c2cccnc2)n1 ; O=C(O)Cc1ccccc1 ; O=C(Cc1ccccc1)Nc1ccn(-c2cccnc2)n1]

### 2-phenyl-N-(1-(pyridin-3-yl)-1H-pyrazol-3-yl)acetamide (Compound 213)

To a solution of 1-(3-pyridyl)pyrazol-3-amine (40.0 mg, 0.25 mmol, 1.0 eq) in DMF (2.0 mL) was added 2-phenylacetic acid (37.4 mg, 0.28 mmol, 1.1 eq), HATU (104.6 mg, 0.28 mmol, 1.1 eq), and iPr₂NEt (131 μL, 0.75 mmol, 3.0 eq). The resultant mixture was stirred at 80° C. for 3 h. The reaction mixture was filtered, and the filtrate concentrated. The crude residue was purified by reverse phase C18 preparatory HPLC (acetonitrile/water with TFA modifier) to provide 2-phenyl-N-(1-(pyridin-3-yl)-1H-pyrazol-3-yl)acetamide (44.3 mg, 64% yield).

### Method C

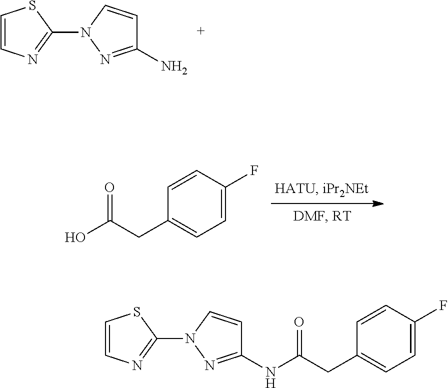 [SMILES: Nc1ccn(-c2nccs2)n1 ; O=C(O)Cc1ccc(F)cc1 ; O=C(Cc1ccc(F)cc1)Nc1ccn(-c2nccs2)n1]

### 2-(4-fluorophenyl)-N-(1-(thiazol-2-yl)-1H-pyrazol-3-yl)acetamide (Compound 92)

To a room temperature solution of 1-thiazol-2-ylpyrazol-3-amine (30 mg, 0.18 mmol, 1.0 eq) in DMF (1.0 mL) was added 2-(4-fluorophenyl)acetic acid (30 mg, 0.19 mmol, 1.1 eq), HATU (70 mg, 0.18 mmol, 1.0 eq), and iPr₂NEt (150 μL, 0.86 mmol, 4.8 eq). The resultant mixture was stirred at room temperature for 16 h. The reaction mixture was partitioned between saturated aqueous NaCl and dichloromethane. The layers were separated, and the organics were dried (Na₂SO₄), filtered, and concentrated. The crude residue was purified by C18 preparatory HPLC (acetonitrile/water using TFA modifier). The material thus obtained was dissolved in dichloromethane and washed with saturated aqueous sodium bicarbonate. The phases were separated on a phase separation cartridge. The organic fraction was concentrated to provide 2-(4-fluorophenyl)-N-(1-(thiazol-2-yl)-1H-pyrazol-3-yl)acetamide (16.3 mg, 28% yield).

### Method D

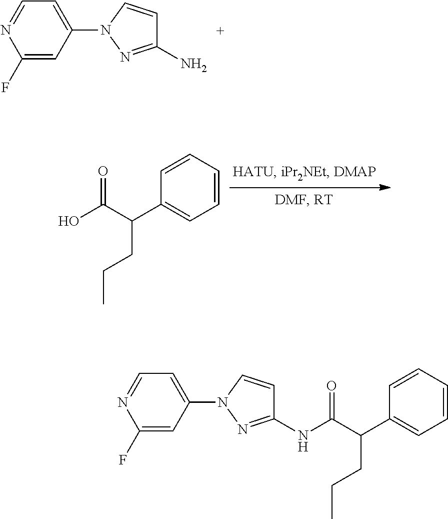 [SMILES: Nc1ccn(-c2ccnc(F)c2)n1 ; CCCC(C(=O)O)c1ccccc1 ; CCCC(C(=O)Nc1ccn(-c2ccnc(F)c2)n1)c1ccccc1]

### N-(1-(2-fluoropyridin-4-yl)-1H-pyrazol-3-yl)-2-phenylpentanamide (Compound 173)

To a room temperature solution of 2-phenylpentanoic acid (60 mg, 0.34 mmol, 1.5 eq) in DMF (2.0 mL) was added HATU (171 mg, 0.45 mmol, 2.0 eq), DMAP (0.3 mg, 0.002 mmol, 0.01 eq), iPr₂NEt (98 μL, 0.56 mmol, 2.5 eq), and 1-(2-fluoropyridin-4-yl)-1H-pyrazol-3-amine (40 mg, 0.22 mmol, 1.0 eq). The resultant mixture was stirred at room temperature for 16 h. The mixture was partitioned between dichloromethane and water. The layers were separated via a phase separation cartridge, and the organics concentrated. The crude residue was purified by C18 preparatory HPLC (acetonitrile/water with TFA modifier). The material thus obtained was dissolved in dichloromethane and passed through a bicarbonate cartridge. The filtrate was concentrated to provide N-(1-(2-fluoropyridin-4-yl)-1H-pyrazol-3-yl)-2-phenylpentanamide (36.8 mg, 48% yield).

### Method E

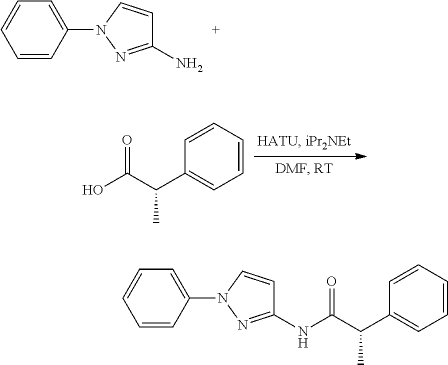 [SMILES: Nc1ccn(-c2ccccc2)n1 ; C[C@H](C(=O)O)c1ccccc1 ; C[C@H](C(=O)Nc1ccn(-c2ccccc2)n1)c1ccccc1]

### (S)-2-phenyl-N-(1-phenyl-1H-pyrazol-3-yl)propanamide (Compound 20)

To a room temperature solution of 1-phenyl-1H-pyrazol-3-amine (60 mg, 0.38 mmol, 1.0 eq) in DMF (2.0 mL) was added (S)-2-phenylpropanoic acid (75 mg, 0.50 mmol, 1.3 eq), HATU (160 mg, 0.42 mmol, 1.1 eq), and iPr₂NEt (200 μL, 1.15 mmol, 3.0 eq). The resultant reaction mixture was stirred at room temperature for 16 h. The reaction mixture was partitioned between ethyl acetate and water. The layers were separated, and the ethyl acetate layer was dried (Na₂SO₄), filtered, and concentrated. The crude residue was purified by reverse phase C18 preparatory HPLC (acetonitrile/water with TFA modifier) to furnish (S)-2-phenyl-N-(1-phenyl-1H-pyrazol-3-yl)propanamide (66 mg, 58% yield).

### Method F

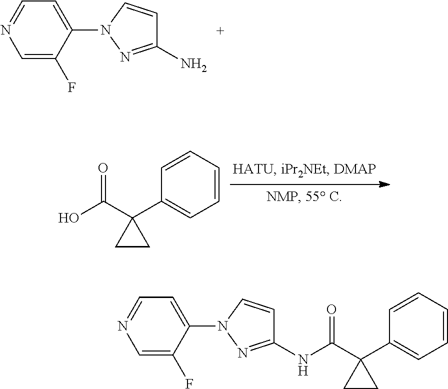 [SMILES: Nc1ccn(-c2ccncc2F)n1 ; O=C(O)C1(c2ccccc2)CC1 ; O=C(Nc1ccn(-c2ccncc2F)n1)C1(c2ccccc2)CC1]

### N-(1-(3-fluoropyridin-4-yl)-1H-pyrazol-3-yl)-1-phenylcyclopropane-1-carboxamide (Compound 138)

To a solution of 1-(3-fluoropyridin-4-yl)-1H-pyrazol-3-amine (25 mg, 0.13 mmol, 1.0 eq) in NMP (1.0 mL) was added 1-phenylcyclopropane-1-carboxylic acid (26 mg, 0.16 mmol, 1.2 eq), HATU (76 mg, 0.20 mmol, 1.5 eq), DMAP (0.8 mg, 0.007 mmol, 0.05 eq), and iPr₂NEt (100 μL, 0.57 mmol, 4.3 eq). The mixture was heated to 55° C. and stirred for 16 h. The reaction mixture was partitioned between saturated aqueous NaCl, saturated NaHCO₃, and dichloromethane (1:1:1). The layers were separated via a phase separation cartridge, and the organics were concentrated. The crude residue was purified by C18 preparatory HPLC (acetonitrile/water with TFA modifier). The material thus obtained was dissolved in dichloromethane and washed with NaHCO₃. The layers were separated, and the organic phase concentrated to provide N-(1-(3-fluoropyridin-4-yl)-1H-pyrazol-3-yl)-1-phenylcyclopropane-1-carboxamide (6.5 mg, 14% yield).

### Method G

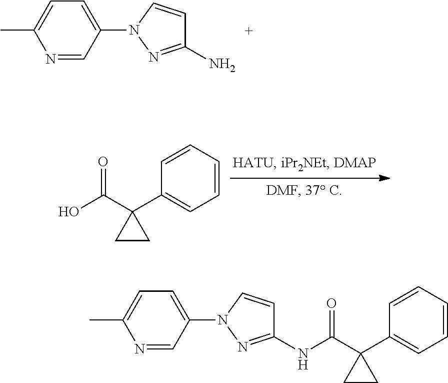 [SMILES: Cc1ccc(-n2ccc(N)n2)cn1 ; O=C(O)C1(c2ccccc2)CC1 ; Cc1ccc(-n2ccc(NC(=O)C3(c4ccccc4)CC3)n2)cn1]

### N-(1-(6-methylpyridin-3-yl)-1H-pyrazol-3-yl)-1-phenylcyclopropane-1-carboxamide (Compound 118)

A mixture of 1-(6-methylpyridin-3-yl)-1H-pyrazol-3-amine (40 mg, 0.23 mmol, 1.0 eq), 1-phenylcyclopropane-1-carboxylic acid (60 mg, 0.37 mmol, 1.6 eq), DMAP (3.0 mg, 0.02 mmol, 0.05 eq), iPr₂NEt (200 μL, 1.15 mmol, 5.0 eq) and HATU (140 mg, 0.37 mmol, 1.6 eq) in DMF (4.0 mL) was stirred for 24 h at 37° C. The reaction mixture was partitioned between saturated aqueous NaHCO₃ and dichloromethane. The layers were separated via a phase separation cartridge, and the organics were concentrated. The crude residue was purified by silica gel chromatography (12 g silica column; linear gradient of 10-50% ethyl acetate/heptane to provide N-(1-(6-methylpyridin-3-yl)-1H-pyrazol-3-yl)-1-phenylcyclopropane-1-carboxamide (44.8 mg, 58% yield).

### Method H

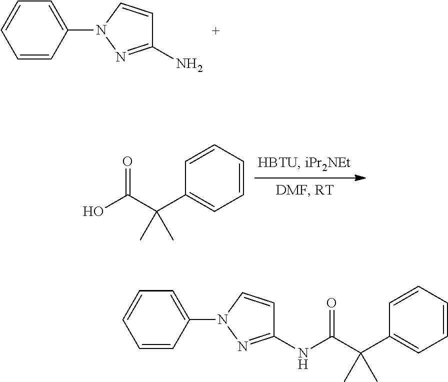 [SMILES: Nc1ccn(-c2ccccc2)n1 ; CC(C)(C(=O)O)c1ccccc1 ; CC(C)(C(=O)Nc1ccn(-c2ccccc2)n1)c1ccccc1]

### 2-methyl-2-phenyl-N-(1-phenyl-1H-pyrazol-3-yl)propanamide (Compound 30)

To a solution of 1-phenyl-1H-pyrazol-3-amine (60 mg, 0.38 mmol, 1.0 eq) and 2-methyl-2-phenylpropanoic acid (62 mg, 0.38 mmol, 1.0 eq) in DMF (2.0 mL) was added HBTU (143 mg, 0.38 mmol, 1.0 eq) and iPr₂NEt (66 μL, 0.38 mmol, 1.0 eq). The resultant reaction mixture was stirred for 18 h at room temperature. The reaction mixture was partitioned between ethyl acetate and water. The layers were separated, and the organic layer was concentrated. The crude residue thus obtained was purified by C18 preparatory HPLC (acetonitrile/water with TFA modifier) to provide 2-methyl-2-phenyl-N-(1-phenyl-1H-pyrazol-3-yl)propanamide (66 mg, 56% yield).

### Method I

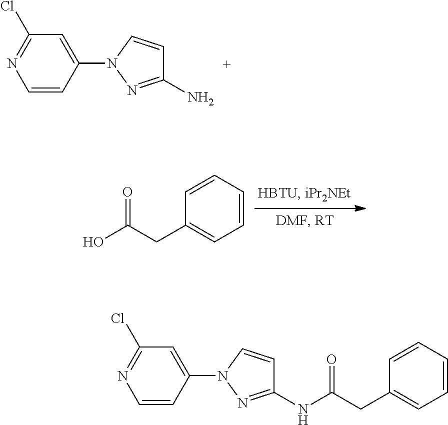 [SMILES: Nc1ccn(-c2ccnc(Cl)c2)n1 ; O=C(O)Cc1ccccc1 ; O=C(Cc1ccccc1)Nc1ccn(-c2ccnc(Cl)c2)n1]

### N-(1-(2-chloropyridin-4-yl)-1H-pyrazol-3-yl)-2-phenylacetamide (Compound 83)

To a solution of 1-(2-chloropyridin-4-yl)-1H-pyrazol-3-amine (100 mg, 0.34 mmol, 1.0 eq) and 2-phenylacetic acid (66 mg, 0.48 mmol, 1.4 eq) in DMF (2.0 mL) was added HBTU (182 mg, 0.48 mmol, 1.4 eq) and iPr₂NEt (180 μL, 1.03 mmol, 3.0 eq). The resultant reaction mixture was stirred for 24 h at room temperature. The reaction mixture was partitioned between saturated aqueous NaCl and dichloromethane. The layers were separated via a phase separation cartridge, and the dichloromethane layer was concentrated. The crude residue was purified by silica gel chromatography (linear gradient of 0-50% ethyl acetate/heptane) to provide N-(1-(2-chloropyridin-4-yl)-1H-pyrazol-3-yl)-2-phenylacetamide (51.6 mg, 46% yield).

### Method J

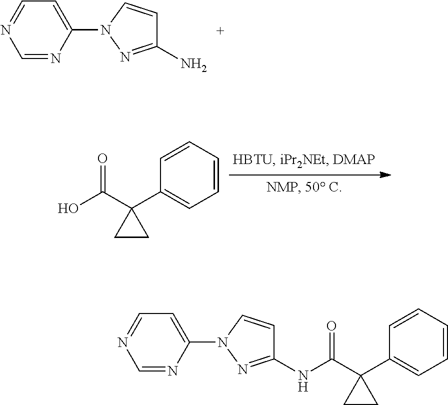 [SMILES: Nc1ccn(-c2ccncn2)n1 ; O=C(O)C1(c2ccccc2)CC1 ; O=C(Nc1ccn(-c2ccncn2)n1)C1(c2ccccc2)CC1]

### 1-phenyl-N-(1-(pyrimidin-4-yl)-1H-pyrazol-3-yl)cyclopropane-1-carboxamide (Compound 207)

To a solution of 1-(pyrimidin-4-yl)-1H-pyrazol-3-amine (25 mg, 0.15 mmol, 1.0 eq) and 1-phenylcyclopropane-1-carboxylic acid (36 mg, 0.22 mmol, 1.5 eq) in NMP (500 μL) was added HBTU (140 mg, 0.37 mmol, 2.5 eq) and iPr₂NEt (51 μL, 0.29 mmol, 2.0 eq). The resultant reaction mixture was stirred for 24 h at 50° C. The reaction mixture was diluted with saturated aqueous NaHCO₃ and saturated aqueous NaCl (1:1), and extracted with dichloromethane. The layers were separated via a phase separation cartridge, and the dichloromethane layer was concentrated. The crude residue was purified by C18 preparatory HPLC (acetonitrile/water with TFA modifier). The material thus obtained was dissolved in dichloromethane and washed with saturated aqueous sodium bicarbonate and dichloromethane. The layers were separated on a phase separation cartridge, and the organic layer was concentrated in vacuo to furnish 1-phenyl-N-(1-(pyrimidin-4-yl)-1H-pyrazol-3-yl)cyclopropane-1-carboxamide (7.6 mg, 16% yield).

### Method K

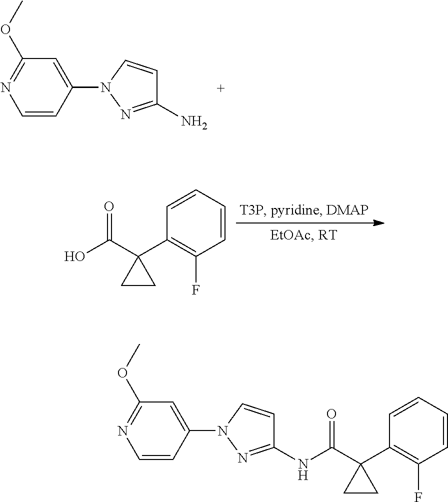 [SMILES: COc1cc(-n2ccc(N)n2)ccn1 ; O=C(O)C1(c2ccccc2F)CC1 ; COc1cc(-n2ccc(NC(=O)C3(c4ccccc4F)CC3)n2)ccn1]

1-(2-fluorophenyl)-N-(1-(2-methoxypyridin-4-yl)-1H-pyrazol-3-yl)cyclopropane-1-carboxamide (Compound 259)

To a 0° C. mixture of 1-(2-methoxypyridin-4-yl)-1H-pyrazol-3-amine (40 mg, 0.21 mmol, 1.0 eq), DMAP (4.0 mg, 0.03 mmol, 0.1 eq), 1-(2-fluorophenyl)cyclopropane-1-carboxylic acid (40 mg, 0.22 mmol, 1.1 eq), and pyridine (80 μL, 0.99 mmol, 4.7 eq) in ethyl acetate (500 μL) was added T3P (50% w/v solution in ethyl acetate, 330 μL, 0.52 mmol, 2.5 eq) dropwise. The resultant solution was allowed to warm to room temperature and stir for 24 h. The reaction mixture was partitioned between saturated aqueous NaCl and dichloromethane. The layers were separated via a phase separation cartridge, and the organics were concentrated. The crude residue was purified by silica gel chromatography (ethyl acetate/heptane) to provide 1-(2-fluorophenyl)-N-(1-(2-methoxypyridin-4-yl)-1H-pyrazol-3-yl)cyclopropane-1-carboxamide (18.8 mg, 24% yield).

### Method L

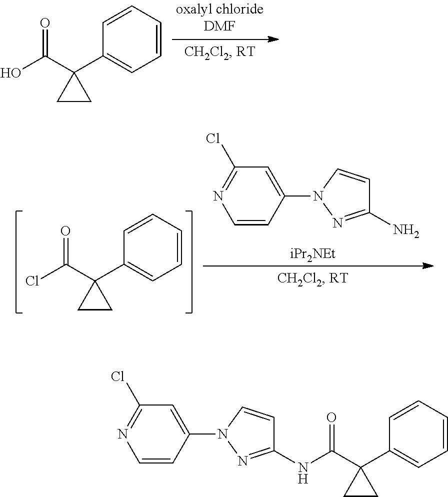

### N-(1-(2-chloropyridin-4-yl)-1H-pyrazol-3-yl)-1-phenylcyclopropane-1-carboxamide (Compound 84)

To a 0° C. solution of 1-phenylcyclopropane-1-carboxylic acid (250 mg, 1.54 mmol, 2.5 eq) in dichloromethane (5.0 mL) was cautiously added oxalyl chloride (150 μL, 1.72 mmol, 1.7 eq) and DMF (10 μL, 0.13 mmol, 0.1 eq). The resultant solution was warmed to room temperature and stirred for 1 h. Meanwhile, 1-(2-chloropyridin-4-yl)-1H-pyrazol-3-amine (300 mg, 1.02 mmol, 1.0 eq) was dissolved in dichloromethane (10.0 mL) and cooled to 0° C. To the resultant mixture was treated with the solution of acid chloride, followed by iPr₂NEt (500 μL, 2.87 mmol, 2.8 eq). The resultant mixture was stirred at room temperature for 24 h then partitioned between saturated aqueous NaHCO₃ and dichloromethane. The biphasic mixture was filtered through a pad of Celite, and the filtrate layers were separated via a phase separation cartridge. The organic phase was concentrated, and the crude residue was purified by silica gel chromatography (linear gradient of ethyl acetate/heptane) to furnish N-(1-(2-chloropyridin-4-yl)-1H-pyrazol-3-yl)-1-phenylcyclopropane-1-carboxamide (80.9 mg, 23% yield).

### Method M

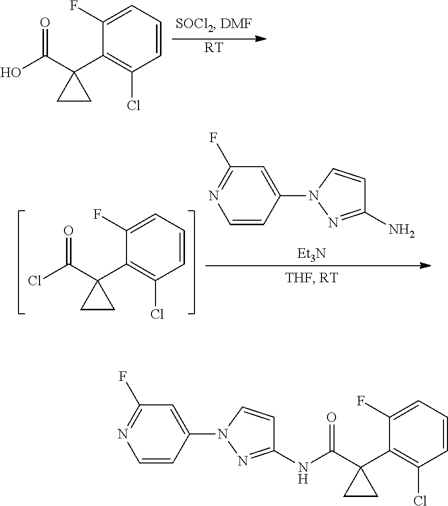

### 1-(2-chloro-6-fluorophenyl)-N-(1-(2-fluoropyridin-4-yl)-1H-pyrazol-3-yl)cyclopropane-1-carboxamide (Compound 274)

### Step 1

To a room temperature solution/suspension of 1-(2-chloro-6-fluorophenyl)cyclopropane-1-carboxylic acid (250 mg, 1.17 mmol, 1.0 eq) in thionyl chloride (255 μL, 3.50 mmol, 3.0 eq) was added DMF (5 μL, 0.06 mmol, 0.05 eq). The resultant reaction solution was stirred for 2 h and concentrated to furnish 1-(2-chloro-6-fluorophenyl)cyclopropane-1-carbonyl chloride which was used in the following step without further purification.

### Step 2

To a room temperature solution of 1-(2-chloro-6-fluorophenyl)cyclopropane-1-carbonyl chloride (50 mg, 0.21 mmol, 1.0 eq) in THF (1.0 ml) was added triethylamine (60 μL, 0.43 mmol, 2.0 eq) and 1-(2-fluoropyridin-4-yl)-1H-pyrazol-3-amine (54 mg, 0.30 mmol, 1.4 eq). The resultant reaction mixture was stirred at room temperature for 24 h. The solvent was removed, and the crude residue was dissolved in DMSO (2.0 mL) and purified by C18 preparatory HPLC (acetonitrile/water with NH₄OH modifier) to provide 1-(2-chloro-6-fluorophenyl)-N-(1-(2-fluoropyridin-4-yl)-1H-pyrazol-3-yl)cyclopropane-1-carboxamide (25.0 mg, 30% yield).

### Method N

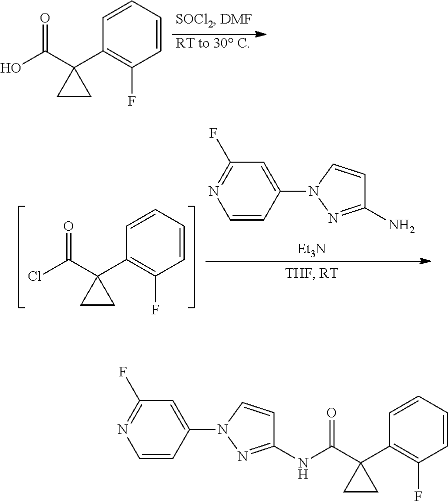

### 1-(2-fluorophenyl)-N-(1-(2-fluoropyridin-4-yl)-1H-pyrazol-3-yl)cyclopropane-1-carboxamide (Compound 87)

### Step 1

To a solution/suspension of 1-(2-fluorophenyl)cyclopropane-1-carboxylic acid (266 g, 1.46 mol, 1.3 eq) in thionyl chloride (SOCl₂; 295 mL, 4.04 mol, 3.6 eq) at room temperature was added DMF (800 μL, 10.33 mmol, 0.01 eq). The resultant solution was stirred 1 hour (h) at room temperature and 3 h at 30° C. The solvent was removed in vacuo, and excess thionyl chloride and HCl were removed by azeotrope with toluene (100 mL). 1-(2-fluorophenyl)cyclopropanecarbonyl chloride (290 g, 100%) was obtained as a clear yellow oil. 1H NMR (400 MHz, CDCl3) δ 7.44-7.24 (m, 2H), 7.24-7.05 (m, 2H), 2.11-1.96 (m, 2H), 1.59-1.43 (m, 2H) ppm. ESI-MS m/z calc. 198.02, found 199.63 (M+1)+.

### Step 2

To a 0° C. suspension of 1-(2-fluoropyridin-4-yl)-1H-pyrazol-3-amine (200 g, 1.12 mol, 1.0 eq) and triethylamine (Et₃N; 391 mL, 2.81 mol, 2.5 eq) in THF (1.6 L) was added 1-(2-fluorophenyl)cyclopropanecarbonyl chloride (290 g, 1.46 mol, 1.3 eq) slowly over 1 h so as to maintain the reaction temperature below 8° C. The reaction mixture was stirred a further for 1 h in the ice-bath then warmed to room temperature for approximately 16 h. After water (200 mL) was added and stirred for about 20 minutes, the THF was removed in vacuo. The resultant mixture was partitioned between ethyl acetate (6.5 L) and aqueous 5% Na₂CO₃ (3 L). The layers were separated, and the organic layer was washed with aqueous 5% Na₂CO₃ (3 L), dried and concentrated. The crude residue was purified by silica gel chromatography (linear gradient of 0-100% ethyl acetate/heptane). Relevant fractions were combined and concentrated to provide the desired product, which was re-suspended in heptane (4 L) and circulated on a rotary evaporator at atmospheric pressure for approximately 16 h. The product was collected by filtration, washed twice with heptane, and dried in vacuo to provide 1-(2-fluorophenyl)-N-(1-(2-fluoropyridin-4-yl)-1H-pyrazol-3-yl)cyclopropane-1-carboxamide (300 g, 78% yield; white crystalline solid). 1H-NMR (400 MHz, DMSO-d6) δ 9.59 (s, 1H), 8.63 (d, J=2.8 Hz, 1H), 8.25 (d, J=5.7 Hz, 1H), 7.71 (dt, J=5.7, 1.5 Hz, 1H), 7.55-7.44 (m, 2H), 7.44-7.33 (m, 1H), 7.28-7.13 (m, 2H), 6.88 (d, J=2.8 Hz, 1H), 1.71-1.54 (m, 2H), 1.25-1.08 (m, 2H) ppm.

### Method N (Alternate)

### 1-(2-fluorophenyl)-N-(1-(2-fluoropyridin-4-yl)-1H-pyrazol-3-yl)cyclopropane-1-carboxamide (Compound 87, Alternate Synthesis)

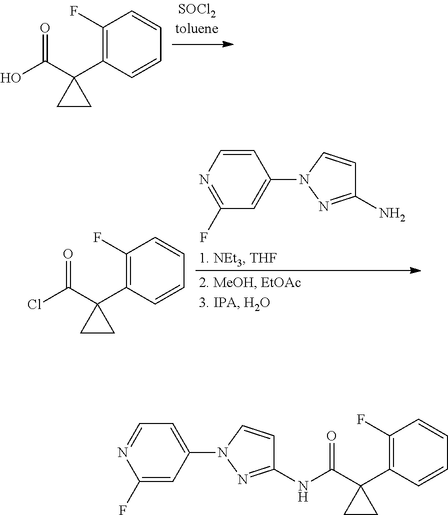

### Step 1

A reactor was charged with 1-(2-fluorophenyl)cyclopropane-1-carboxylic acid (1750.6 g, 9.72 mol, limiting reagent), and toluene (3.5 L, 2 vol) was added. Thionyl chloride (1417 mL, 19.43 mol, 2 eq) was added to reactor, and the reaction was heated to 35-40° C. Upon completion of the reaction, toluene (7 L, 4 vol) was added to the reactor, and the reaction mixture was distilled to dryness to obtain 1-(2-fluorophenyl)cyclopropanecarbonyl chloride in 98% yield as a yellow oil.

### Step 2

A reactor was charged with 1-(2-fluoropyridin-4-yl)-1H-pyrazol-3-amine (1499.9 g, 8.42 mol, limiting reagent) and tetrahydrofuran (15 L, 10 vol). Triethylamine (2.35 L, 16.84 mol, 2 eq) was added at 13° C. A solution of 1-(2-fluorophenyl)cyclopropanecarbonyl chloride (1672.4 g, 8.42 mol, 1.0 eq) in tetrahydrofuran (3.0 L, 2 vol) was added to the reactor, while maintaining a temperature of 13-18° C. Upon reaction completion, methanol (0.75 L0.5 vol) was added, and the mixture was stirred for no less than 30 minutes. Water (6 L, 4 vol) was added to the reactor at 14° C., and the mixture was allowed to warm up to ambient temperature. The reaction mixture was extracted with ethyl acetate (7.5 L, 5 vol), and the organic layer was washed with 1 N HCl (6.76 L, 4.5 vol), followed by water (6 L, 4 vol). The organic layer was concentrated, isopropyl alcohol (11.25 L, 7.5 vol.) was added, and the mixture was heated to 75° C. Water (3.8 L, 2.5 vol) was added to the reactor over 1 h, while maintaining a temperature greater than 70° C. Seed crystals of 1-(2-fluorophenyl)-N-(1-(2-fluoropyridin-4-yl)-1H-pyrazol-3-yl)cyclopropane-1-carboxamide (28.7 g, 0.08 mol, 0.01 eq) were added at 55° C., and the mixture was stirred for 30 minutes. Water (7.5 L, 5 vol) was added to the reactor at 50-55° C. over 5 h, and then the jacket was ramped down to 20° C. over 5 hours. Stirring was continued at 20° C. for 30 minutes, and then the batch was filtered and washed with 1:1 isopropyl alcohol:water (3.8 L). The wet cake was transferred to drying trays and dried in a vacuum oven at 45° C. with nitrogen bleed. 1-(2-fluorophenyl)-N-(1-(2-fluoropyridin-4-yl)-1H-pyrazol-3-yl)cyclopropane-1-carboxamide was obtained in 83.5% yield.

### Method O

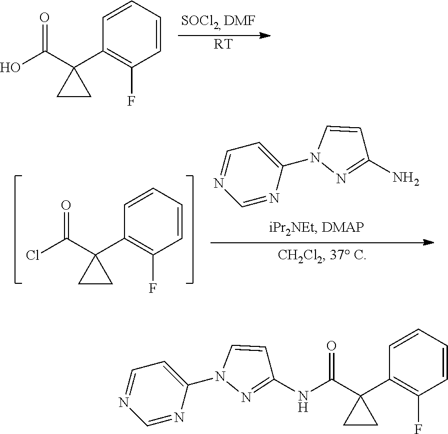

### 1-(2-fluorophenyl)-N-(1-(pyrimidin-4-yl)-1H-pyrazol-3-yl)cyclopropane-1-carboxamide (Compound 206)

### Step 1

1-(2-fluorophenyl)cyclopropane-1-carbonyl chloride was prepared according to procedure described for Method M, Step 1.

### Step 2

A mixture of 1-(pyrimidin-4-yl)-1H-pyrazol-3-amine (50 mg, 0.31 mmol, 1.0 eq), 1-(2-fluorophenyl)cyclopropane-1-carbonyl chloride (70 mg, 0.35 mmol, 1.1 eq), iPr₂NEt (250 μL, 1.44 mmol, 4.6 eq), and DMAP (10 mg, 0.08 mmol, 0.3 eq) in THF (2.0 mL) was heated to 37° C. for 24 h. The solvent was removed, and the crude residue was purified by silica gel chromatography (linear gradient of 10-100% ethyl acetate/heptane) to provide 1-(2-fluorophenyl)-N-(1-(pyrimidin-4-yl)-1H-pyrazol-3-yl)cyclopropane-1-carboxamide (25.1 mg, 25% yield).

### Method P

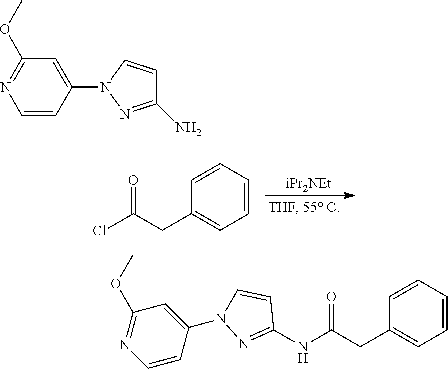

### N-(1-(2-methoxypyridin-4-yl)-1H-pyrazol-3-yl)-2-phenylacetamide (Compound 232)

To a solution of 1-(2-methoxy-4-pyridyl)pyrazol-3-amine (50 mg, 0.26 mmol, 1.0 eq) and iPr₂NEt (200 μL, 1.15 mmol, 4.5 eq) in THF (2.8 mL) was added 2-phenylacetyl chloride (50 μL, 0.40 mmol, 1.6 eq). The resultant mixture was stirred at 55° C. for 1 h then cooled to room temperature and stirred for 16 h. The reaction solution was concentrated, and the crude residue was purified by silica gel chromatography (linear gradient of 10-100% ethyl acetate/heptane) to provide N-(1-(2-methoxypyridin-4-yl)-1H-pyrazol-3-yl)-2-phenylacetamide (40.0 mg, 48% yield).

### Method Q

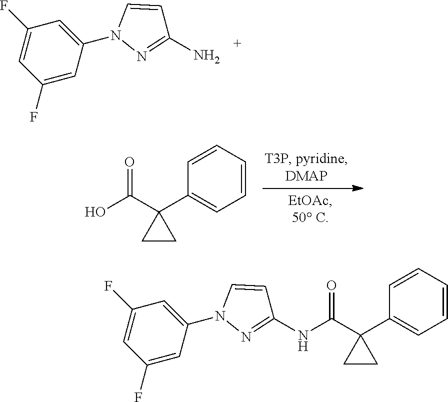 [SMILES: Nc1ccn(-c2cc(F)cc(F)c2)n1 ; O=C(O)C1(c2ccccc2)CC1 ; O=C(Nc1ccn(-c2cc(F)cc(F)c2)n1)C1(c2ccccc2)CC1]

### N-(1-(3,5-difluorophenyl)-1H-pyrazol-3-yl)-1-phenylcyclopropane-1-carboxamide (Compound 308)

A mixture of 1-phenylcyclopropane-1-carboxylic acid (65 mg, 0.40 mmol, 1.6 eq), DMAP (5.0 mg, 0.04 mmol, 0.16 eq), 1-(3,5-difluorophenyl)-1H-pyrazol-3-amine (50 mg, 0.25 mmol, 1.0 eq), and pyridine (200 μL, 2.47 mmol, 9.9 eq) in ethyl acetate (0.5 mL) was cooled to 0° C. To the solution was added T3P (225 μL, 0.35 mmol, 1.4 eq; 50% w/v in ethyl acetate). The ice bath was removed, and the mixture was warmed to room temperature for 24 h, then 50° C. for 24 h. The reaction mixture was cooled to room temperature and partitioned between saturated aqueous NaCl and dichloromethane. The layers were separated via a phase separation cartridge, and the organics were concentrated. The crude residue was purified by silica gel chromatography (linear gradient of 0-20% ethyl acetate/heptane to provide N-(1-(3,5-difluorophenyl)-1H-pyrazol-3-yl)-1-phenylcyclopropane-1-carboxamide (6.3 mg, 7% yield).

### Method R

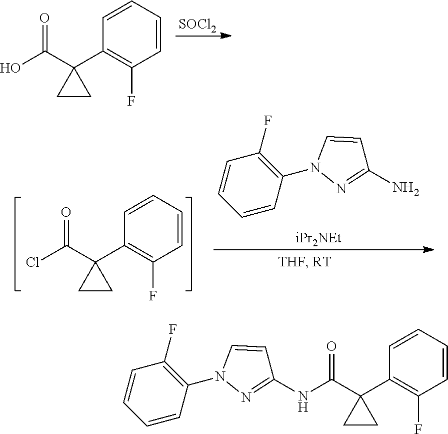

### 1-(2-fluorophenyl)-N-(1-(2-fluorophenyl)-1H-pyrazol-3-yl)cyclopropane-1-carboxamide (Compound 186)

### Step 1

A solution of 1-(2-fluorophenyl)cyclopropane-1-carboxylic acid (50 mg, 0.28 mmol, 1.0 eq) and thionyl chloride (0.5 ml) was heated to reflux for 1 h. The reaction solution was cooled to room temperature and concentrated in vacuo to provide 1-(2-fluorophenyl)cyclopropane-1-carbonyl chloride, which was used in the following step without further manipulation.

### Step 2

To the entirety of the crude 1-(2-fluorophenyl)cyclopropane-1-carbonyl chloride prepared in Step 1 was added THF (2.0 mL), iPr₂NEt (146 μL, 0.84 mmol, 3.0 eq), and 1-(2-fluorophenyl)-1H-pyrazol-3-amine (50 mg, 0.28 mmol, 1.0 eq). The resultant mixture was stirred at room temperature for 30 min. The reaction mixture was filtered, and the filtrate was concentrated. The crude residue was purified by C18 preparatory HPLC (acetonitrile/water with TFA modifier) to provide 1-(2-fluorophenyl)-N-(1-(2-fluorophenyl)-1H-pyrazol-3-yl)cyclopropane-1-carboxamide (7.8 mg, 8% yield).

### Method S

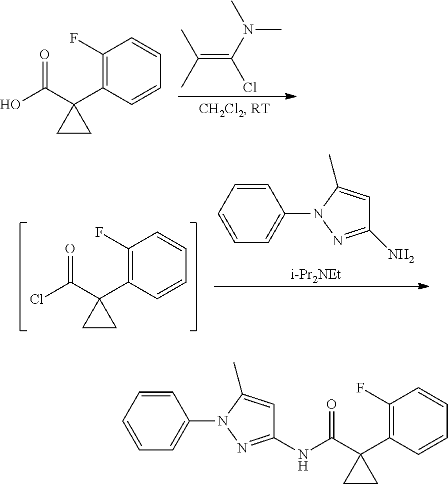

### 1-(2-fluorophenyl)-N-(5-methyl-1-phenyl-1H-pyrazol-3-yl)cyclopropane-1-carboxamide (Compound 386)

To a mixture of 1-(2-fluorophenyl)cyclopropanecarboxylic acid (25 mg, 0.14 mmol, 1.0 eq) in dichloromethane (2.0 mL) was added 1-chloro-N,N,2-trimethylprop-1-en-1-amine (22 μL, 0.166 mmol, 1.2 eq). The resultant mixture was stirred for 2 hours, then treated with a solution of 5-methyl-1-phenyl-pyrazol-3-amine (30 mg, 0.173 mmol, 1.3 eq) in dichloromethane (2.0 mL) and N-ethyl-N-isopropylpropan-2-amine (50 μL, 0.287 mmol, 2.1 eq). The reaction mixture was stirred 16 h. The solvent was removed, and the crude residue was purified by C18 preparatory HPLC (acetonitrile/water with TFA modifier). The material thus obtained was dissolved in dichloromethane, washed with saturated sodium bicarbonate solution, dried (Na₂SO₄), filtered and concentrated to provide 1-(2-fluorophenyl)-N-(5-methyl-1-phenyl-1H-pyrazol-3-yl)cyclopropane-1-carboxamide (26.5 mg, 56% yield).

### Method T

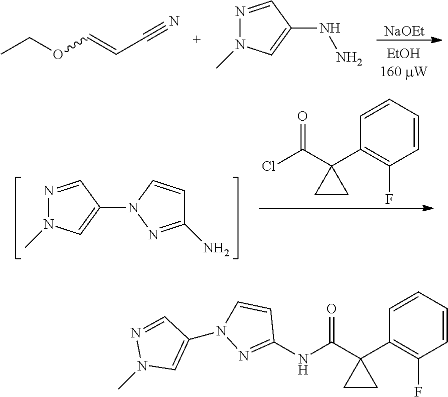

### 1-(2-fluorophenyl)-N-(1′-methyl-1′H-[1,4′-bipyrazol]-3-yl)cyclopropane-1-carboxamide (Compound 390)

A mixture of (E/Z)-3-ethoxyprop-2-enenitrile (50 μL), 1-methylpyrazol-4-yl)hydrazine (dihydrochloride salt; 50 mg, 0.27 mmol, 1.0 eq), sodium ethoxide (500 μL of 21% w/v, 1.54 mmol, 5.7 eq), and ethanol (2.0 mL) was sealed and heated to 160° C. in microwave for 45 mins. The mixture was cooled to room temperature, the solvent evaporated, and the crude residue purified by silica gel chromatography (linear gradient of 0-100% ethyl acetate/heptane) to provide 1-(2-fluorophenyl)-N-(1′-methyl-1′H-[1,4′-bipyrazol]-3-yl)cyclopropane-1-carboxamide (24.6 mg, 25% yield).

### Method U

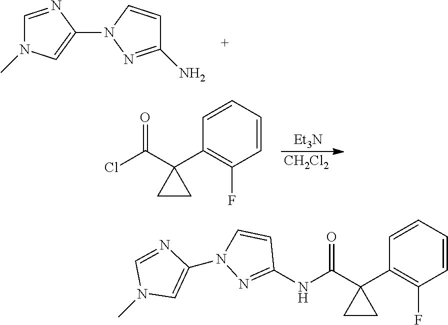

### 1-(2-fluorophenyl)-N-(1-(1-methyl-1H-imidazol-4-yl)-1H-pyrazol-3-yl)cyclopropane-1-carboxamide (Compound 392)

To a solution of 1-(1-methylimidazol-4-yl)pyrazol-3-amine (36 mg, 0.22 mmol, 1.0 eq) in dichloromethane (2.0 mL) was added triethylamine (100 μL, 0.72 mmol, 3.3 eq) and 1-(2-fluorophenyl)cyclopropanecarbonyl chloride (45 mg, 0.23 mmol 1.0 eq). The resultant mixture was stirred for 30 minutes at room temperature. The solvent was evaporated, and the crude residue was purified by silica gel chromatography (linear gradient of methanol/dichloromethane or ethyl acetate/heptane, depending on the product) to provide 1-(2-fluorophenyl)-N-[1-(1-methylimidazol-4-yl)pyrazol-3-yl]cyclopropanecarboxamide (35.3 mg, 47% yield).

### Method V

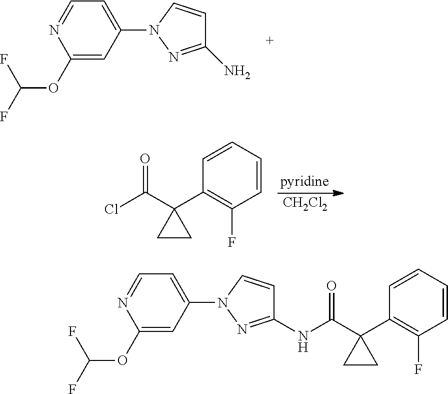

### N-(1-(2-(difluoromethoxy)pyridin-4-yl)-1H-pyrazol-3-yl)-1-(2-fluorophenyl)cyclopropane-1-carboxamide (Compound 395)

To a solution of 1-(2-fluorophenyl)cyclopropanecarbonyl chloride (66 mg, 0.33 mmol, 1.5 eq) in dichloromethane (2.0 mL) was added pyridine (36 μL, 0.44 mmol, 2.0 eq). The resultant mixture was treated with 1-(2-(difluoromethoxy)pyridin-4-yl)-1H-pyrazol-3-amine (50 mg, 0.22 mmol, 1.0 eq) and stirred 16 h. The solvent was evaporated, and the crude residue was purified by C18 preparatory HPLC (acetonitrile/water with NH₄OH modifier) to provide N-(1-(2-(difluoromethoxy)pyridin-4-yl)-1H-pyrazol-3-yl)-1-(2-fluorophenyl)cyclopropane-1-carboxamide (26 mg, 27% yield).

### Method W

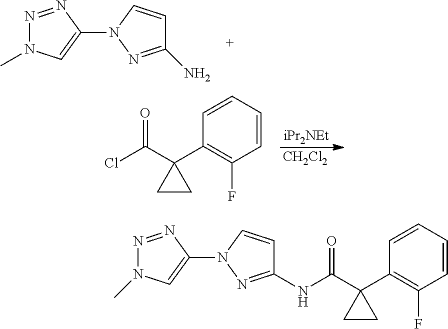

### 1-(2-fluorophenyl)-N-(1-(1-methyl-1H-1,2,3-triazol-4-yl)-1H-pyrazol-3-yl)cyclopropane-1-carboxamide (Compound 400)

To mixture of 1-(1-methyltriazol-4-yl)pyrazol-3-amine (33 mg, 0.20 mmol, 1.0 eq) in dichloromethane (2.0 mL) was added N,N-diisopropylethylamine (100 μL, 0.57 mmol, 2.9 eq) and 1-(2-fluorophenyl)cyclopropanecarbonyl chloride (45 mg, 0.23 mmol, 1.1 eq). The resultant mixture was stirred for 30 minutes at room temperature. The solvent was evaporated, and the crude residue was purified by silica gel chromatography (linear gradient of methanol/dichloromethane) to provide 1-(2-fluorophenyl)-N-(1-(1-methyl-1H-1,2,3-triazol-4-yl)-1H-pyrazol-3-yl)cyclopropane-1-carboxamide (60 mg, 86% yield).

### Method X

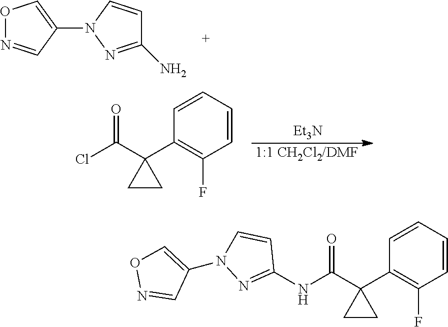

### 1-(2-fluorophenyl)-N-(1-(isoxazol-4-yl)-1H-pyrazol-3-yl)cyclopropane-1-carboxamide (Compound 424)

To a solution of 1-isoxazol-4-ylpyrazol-3-amine (12 mg, 0.08 mmol, 1.0 eq) in dichloromethane (0.5 mL) and DMF (0.5 mL) was added triethylamine (15 μL, 0.11 mmol, 1.4 eq) and 1-(2-fluorophenyl)cyclopropanecarbonyl chloride (79 μL of a 1M solution in dichloromethane, 0.11 mmol, 1.0 eq). The resultant mixture was stirred for 16 h at room temperature. The crude reaction mixture was partitioned between dichloromethane and saturated aqueous sodium bicarbonate. The organics were collected by passage through a phase separation cartridge and evaporated. The crude residue was purified by silica gel chromatography (linear gradient of ethyl acetate/heptane) to provide 1-(2-fluorophenyl)-N-(1-(isoxazol-4-yl)-1H-pyrazol-3-yl)cyclopropane-1-carboxamide (6.8 mg, 26% yield).

### Method Y

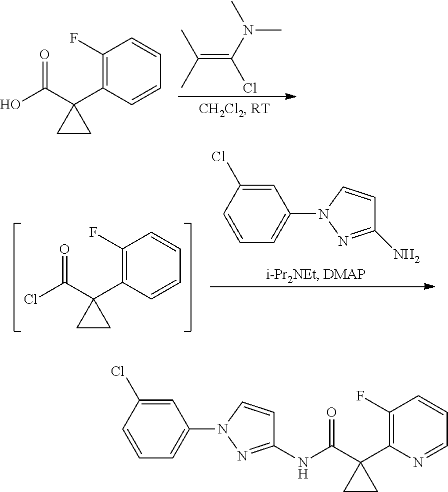

### N-(1-(3-chlorophenyl)-1H-pyrazol-3-yl)-1-(3-fluoropyridin-2-yl)cyclopropane-1-carboxamide (Compound 418)

To a mixture of 1-(3-fluoro-2-pyridyl)cyclopropanecarboxylic acid (46 mg, 0.254 mmol, 1.0 eq) in dichloromethane (2.0 mL) was added 1-chloro-N,N,2-trimethylprop-1-en-1-amine (35 μL, 0.265 mmol, 1.04 eq). The resultant mixture was stirred for 2 hours, then 1-(3-chlorophenyl)pyrazol-3-amine (49 mg, 0.254 mmol, 1.3 eq), N-ethyl-N-isopropylpropan-2-amine (50 μL, 0.287 mmol, 1.1 eq) and DMAP (3 mg, 0.025 mmol, 0.1 eq) were added. The reaction mixture was stirred for 16 h. The solvent was removed, and the crude residue was purified by C18 preparatory HPLC (acetonitrile/water with TFA modifier). The material thus obtained was dissolved in dichloromethane, washed with saturated sodium bicarbonate solution, dried (Na₂SO₄), filtered and concentrated to provide N-(1-(3-chlorophenyl)-1H-pyrazol-3-yl)-1-(3-fluoropyridin-2-yl)cyclopropane-1-carboxamide (22.6 mg, 52% yield).

### Method Z

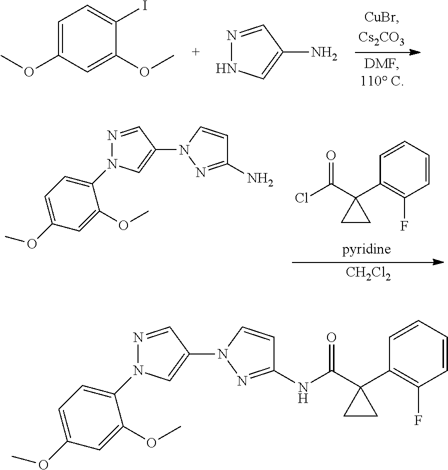

### N-(1′-(2,4-dimethoxyphenyl)-1′H-[1,4′-bipyrazol]-3-yl)-1-(2-fluorophenyl)cyclopropane-1-carboxamide (Compound 428)

### Step 1: 1′-(2,4-dimethoxyphenyl)-1′H-[1,4′-bipyrazol]-3-amine

1H-pyrazol-3-amine (157.4 mg, 1.894 mmol), 1-iodo-2,4-dimethoxy-benzene (500 mg, 1.894 mmol) copper(I) bromide (54.3 mg, 0.379 mmol), cesium carbonate (617.1 mg, 1.894 mmol) and DMF (2.0 mL) were combined and heated to 110° C. overnight. The resultant mixture was cooled to room temperature and passed through a plug of celite, washing with methanol. The filtrate was evaporated, and the crude residue was dissolved in dichloromethane and washed with 1N NaOH. The organics were collected by passage through a phase separation cartridge, and the filtrate was evaporated to provide crude 1′-(2,4-dimethoxyphenyl)-1′H-[1,4′-bipyrazol]-3-amine, a portion of which was used in the following step without further manipulation.

### Step 2: N-(1′-(2,4-dimethoxyphenyl)-1′H-[1,4′-bipyrazol]-3-yl)-1-(2-fluorophenyl)cyclopropane-1-carboxamide

To a solution of crude 1′-(2,4-dimethoxyphenyl)-1′H-[1,4′-bipyrazol]-3-amine (50 mg, 0.175 mmol) in dichloromethane (1.0 mL) was added 1-(2-fluorophenyl)cyclopropanecarbonyl chloride (56.4 mg, 0.283 mmol) and pyridine (153 μL). The resultant solution was stirred for 16 h, and the solvent was then evaporated under a stream of nitrogen gas. The crude residue was dissolved in DMSO and purified by C18 preparatory HPLC (acetonitrile/water with TFA modifier). The material thus obtained was dissolved in dichloromethane, washed with saturated sodium bicarbonate solution, dried (Na₂SO₄), filtered, and concentrated to provide N-(1′-(2,4-dimethoxyphenyl)-1′H-[1,4′-bipyrazol]-3-yl)-1-(2-fluorophenyl)cyclopropane-1-carboxamide (52% yield).

### Method AA

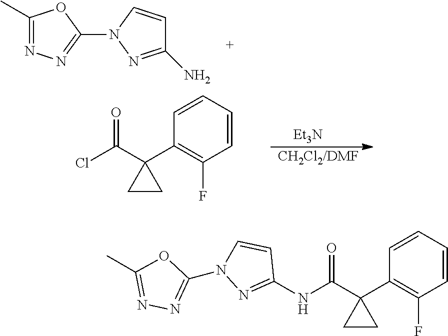

### 1-(2-fluorophenyl)-N-(1-(5-methyl-1,3,4-oxadiazol-2-yl)-1H-pyrazol-3-yl)cyclopropane-1-carboxamide (Compound 443)

To a solution of 1-(5-methyl-1,3,4-oxadiazol-2-yl)-1H-pyrazol-3-amine (60 mg, 0.34 mmol, 1.0 eq) in dichloromethane (0.3 mL) and DMF (1.0 mL) was added triethylamine (100 μL, 0.72 mmol, 2.1 eq) and 1-(2-fluorophenyl)cyclopropanecarbonyl chloride (80 mg, 0.34 mmol, 1.0 eq). The resultant mixture was stirred for 72 h at 60° C. The crude reaction mixture was cooled to room temperature and partitioned between dichloromethane and saturated aqueous sodium bicarbonate. The organics were collected by passage through a phase separation cartridge and evaporated. The crude residue was purified by silica gel chromatography (linear gradient of ethyl acetate/heptane) to provide 1-(2-fluorophenyl)-N-(1-(5-methyl-1,3,4-oxadiazol-2-yl)-1H-pyrazol-3-yl)cyclopropane-1-carboxamide (7.9 mg, 7% yield).

### Method AB

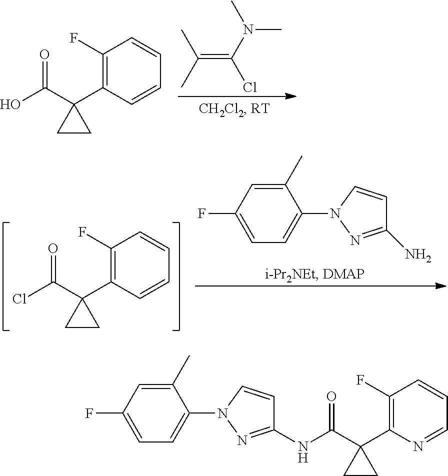

### N-(1-(4-fluoro-2-methylphenyl)-1H-pyrazol-3-yl)-1-(3-fluoropyridin-2-yl)cyclopropane-1-carboxamide (Compound 450)

To a mixture of 1-(3-fluoro-2-pyridyl)cyclopropanecarboxylic acid (50 mg, 0.276 mmol, 1.0 eq) in dichloromethane (1.0 mL) was added 1-chloro-N,N,2-trimethylprop-1-en-1-amine (45 μL, 0.340 mmol, 1.2 eq). The resultant mixture was stirred for 1 h, then treated with 1-(4-fluoro-2-methyl-phenyl)pyrazol-3-amine (55 mg, 0.288 mmol, 1.04 eq), N-ethyl-N-isopropylpropan-2-amine (200 μL, 1.148 mmol, 4.2 eq), and DMAP (10 mg, 0.082 mmol, 0.3 eq). The reaction mixture was stirred 2 h. The solvent was removed, and the crude residue was purified by C18 preparatory HPLC (acetonitrile/water with TFA modifier). The material thus obtained was dissolved in dichloromethane, washed with saturated sodium bicarbonate solution, dried (Na₂SO₄), filtered and concentrated to provide N-(1-(4-fluoro-2-methylphenyl)-1H-pyrazol-3-yl)-1-(3-fluoropyridin-2-yl)cyclopropane-1-carboxamide (10.9 mg, 10% yield).

### Method AC

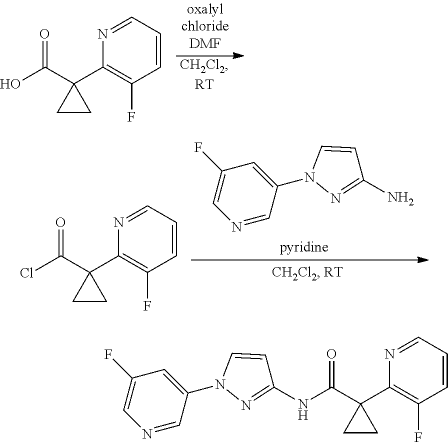

### 1-(3-fluoropyridin-2-yl)-N-(1-(5-fluoropyridin-3-yl)-1H-pyrazol-3-yl)cyclopropane-1-carboxamide (Compound 423)

### Step 1

To a 0° C. mixture of 1-(3-fluoro-2-pyridyl)cyclopropanecarboxylic acid (660 mg, 3.64 mmol) in dichloromethane (10 mL) was added oxalyl chloride (2 mL of 2 M solution in dichloromethane, 4.00 mmol). The resultant reaction solution was treated with N,N-dimethylformamide (25 μL, 0.32 mmol). Stirring at 0° C. was continued for 10 minutes, and then the reaction was warmed to room temperature and stirred for 30 minutes. The solvent was removed in vacuo to furnish 1-(3-fluoropyridin-2-yl)cyclopropane-1-carbonyl chloride as a light yellow solid, the entirety of which was used in the following step without further manipulation.

### Step 2

1-(3-fluoropyridin-2-yl)cyclopropane-1-carbonyl chloride from Step 1 was dissolved in dichloromethane (10 mL) and pyridine (1.0 mL, 12.36 mmol). To the resultant solution was added a suspension of 1-(5-fluoro-3-pyridyl)pyrazol-3-amine (445 mg, 2.50 mmol) in dichloromethane (5 mL). Stirring was continued for 2 h, and then the solvent was removed in vacuo. The crude residue thus obtained was purified by silica gel chromatography (isocratic 5% methanol/dichloromethane) to provide 1-(3-fluoropyridin-2-yl)-N-(1-(5-fluoropyridin-3-yl)-1H-pyrazol-3-yl)cyclopropane-1-carboxamide (655 mg, 77% yield).

### Method AD

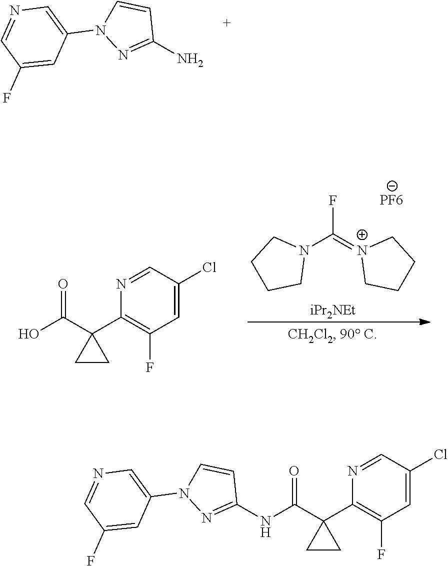 [SMILES: "iPr2NEt" ; Nc1ccn(-c2cncc(F)c2)n1 ; O=C(O)C1(c2ncc(Cl)cc2F)CC1 ; FC(N1CCCC1)=[N+]1CCCC1 ; "PF6" ; O=C(Nc1ccn(-c2cncc(F)c2)n1)C1(c2ncc(Cl)cc2F)CC1]

### 1-(5-chloro-3-fluoropyridin-2-yl)-N-(1-(5-fluoropyridin-3-yl)-1H-pyrazol-3-yl)cyclopropane-1-carboxamide (Compound 498)

1-(5-chloro-3-fluoro-2-pyridyl)cyclopropanecarboxylic acid (TFA salt, 23 mg, 0.068 mmol), N,N-diisopropylethylamine (100 μL, 0.574 mmol), 1-[fluoro(pyrrolidin-1-ium-1-ylidene)methyl]pyrrolidine (Phosphorus Hexafluoride Ion, 40 mg, 0.127 mmol), and dichlormethane (2.0 mL) were combined. The resultant mixture was stirred for 30 minutes. 1-(5-fluoro-3-pyridyl)pyrazol-3-amine (12 mg, 0.067 mmol) was added, and the reaction vessel was sealed and heated to 90° C. for 4 hours. The solvent was evaporated, and the crude residue was dissolved in a small amount of DMSO and purified by C18 preparatory HPLC (acetonitrile/water with TFA or NH₄OH modifier) to provide 1-(5-chloro-3-fluoropyridin-2-yl)-N-(1-(5-fluoropyridin-3-yl)-1H-pyrazol-3-yl)cyclopropane-1-carboxamide (12.8 mg, 45% yield).

### Method AE

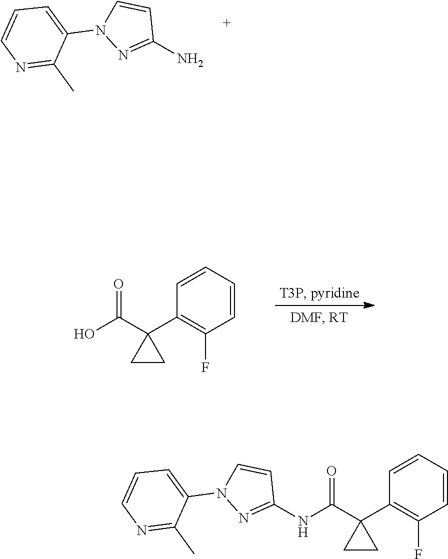 [SMILES: Cc1ncccc1-n1ccc(N)n1 ; O=C(O)C1(c2ccccc2F)CC1 ; "T3P, pyridine" ; Cc1ncccc1-n1ccc(NC(=O)C2(c3ccccc3F)CC2)n1]

### 1-(2-fluorophenyl)-N-(1-(2-methylpyridin-3-yl)-1H-pyrazol-3-yl)cyclopropane-1-carboxamide (Compound 470)

1-(2-fluorophenyl)cyclopropanecarboxylic acid (56.9 mg, 0.316 mmol, 1.1 eq), pyridine (46 μL, 0.574 mmol, 2.0 eq) and DMF (1.0 mL) were combined. T3P (215 μL of a 2M solution in ethyl acetate, 0.431 mmol, 1.5 eq) was added, and stirring was continued for 5 minutes prior to addition of 1-(2-methyl-3-pyridyl)pyrazol-3-amine (50 mg, 0.287 mmol, 1.0 eq). The reaction mixture was stirred overnight and diluted with dichloromethane and water. The organic phase was collected by passage through phase separator, and the filtrate was concentrated. The crude residue was purified by silica gel chromatography (linear gradient of 0-40% ethyl acetate/heptane to provide 1-(2-fluorophenyl)-N-(1-(2-methylpyridin-3-yl)-1H-pyrazol-3-yl)cyclopropane-1-carboxamide (20.5 mg, 21% yield).

TABLE A
Compounds prepared using amide bond formation as the final step.

| | | | | Average | | NMR |
|---|---|---|---|---|---|---|
| | | Cou- | | HEK293 | | (shifts in ppm) and |
| Cpd | | pling | Yield | IC50 | | Optical Rotation |
| No. | Structure | Method | (%) | (uM)* | M + 1 | (where applicable) |
| 1 | 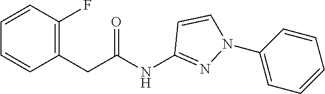 [SMILES: O=C(Cc1ccccc1F)Nc1ccn(-c2ccccc2)n1] | A | 49 | 0.068 | 296.11 | 1H NMR (400 MHz, DMSO-d₆) δ 11.01 (s, 1H), 8.41 (d, J = 2.6 Hz, 1H), 7.78 (dd, J = 8.6, 0.9 Hz, 2H), 7.52-7.46 (m, 2H), 7.40 (td, J = 7.6, 1.5 Hz, 1H), 7.31 (ddd, J = 12.1, |
| | | | | | | 7.1, 4.5 Hz, 2H), 7.20- |
| | | | | | | 7.15 (m, 2H), 6.76 (d, J = |
| | | | | | | 2.5 Hz, 1H), 3.76 (s, 2H). |
| 2 | 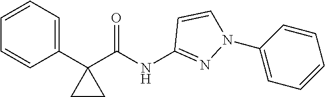 [SMILES: O=C(Nc1ccn(-c2ccccc2)n1)C1(c2ccccc2)CC1] | A | 59 | 0.020 | 304.19 | 1H NMR (300 MHz, CDCl₃) δ 7.79 (d, J = 2.5 Hz, 1H), 7.72 (s, 1H), 7.66-7.50 (m, 3H), 7.50-7.34 (m, 5H), 7.29-7.18 (m, 2H), 6.96 (d, J = 2.5 Hz, 1H), 1.82-1.70 (m, 2H), 1.27- |
| | | | | | | 1.14 (m, 2H). |
| 3 | 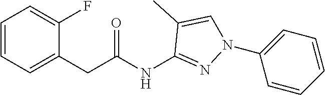 [SMILES: Cc1cn(-c2ccccc2)nc1NC(=O)Cc1ccccc1F] | E | 49 | RND | 310.14 | 1H NMR (300 MHz, CDCl₃) δ 7.67 (s, 1H), 7.65-7.52 (m, 2H), 7.42 (m, 3H), 7.40- 7.02 (m, 4H), 3.78 (d, J = 23.5 Hz, 2H), 2.07 (s, 3H). |
| 4 | 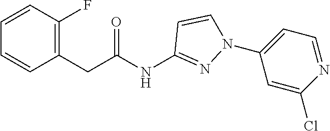 [SMILES: O=C(Cc1ccccc1F)Nc1ccn(-c2ccnc(Cl)c2)n1] | E | 33 | 0.65 | 331.00 | 1H NMR (300 MHz, DMSO-d₆) δ 11.14 (s, 1H), 8.66 (d, J = 2.8 Hz, 1H), 8.44 (d, J = 5.7 Hz, 1H), 7.88 (d, J = 1.7 Hz, 1H), 7.77 (dd, J = 5.7, 2.0 Hz, 1H), 7.46-7.25 (m, 2H), 7.23-7.00 (m, 2H), 6.87 (d, J = 2.7 Hz, 1H), 3.76 (s, |
| | | | | | | 2H). |
| 5 | 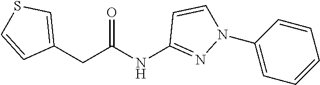 [SMILES: O=C(Cc1ccsc1)Nc1ccn(-c2ccccc2)n1] | A | 16 | 0.41 | 284.22 | ¹H NMR (400 MHz, DMSO- d₆) δ 10.91 (s, 1H), 8.37 (d, J = 2.6 Hz, 1H), 7.74 (d, J = 8.1 Hz, 2H), 7.53-7.42 (m, 3H), 7.32 (d, J = 2.9 Hz, 1H), 7.26 (t, J = 7.4 Hz, |
| | | | | | | 1H), 7.08 (d, J = 4.9 Hz, |
| | | | | | | 1H), 6.76 (d, J = 2.5 Hz, |
| | | | | | | 1H), 4.12 (d, J = 5.4 Hz, |
| | | | | | | 2H). |
| 6 | 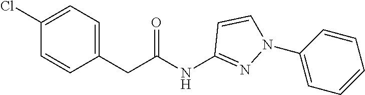 [SMILES: O=C(Cc1ccc(Cl)cc1)Nc1ccn(-c2ccccc2)n1] | A | 37 | >5 | 312.08 | 1H NMR (400 MHz, DMSO-d₆) δ 11.00 (s, 1H), 8.39 (d, J = 2.6 Hz, 1H), 7.76 (d, J = 8.0 Hz, 2H), 7.49 (t, J = 7.8 Hz, 2H), 7.42-7.33 (m, 4H), 7.27 (t, J = 7.4 Hz, 1H), 6.75 (d, |
| | | | | | | J = 2.5 Hz, 1H), 3.67 (s, |
| | | | | | | 2H). |
| 7 | 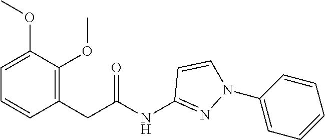 [SMILES: COc1cccc(CC(=O)Nc2ccn(-c3ccccc3)n2)c1OC] | A | 62 | >5 | 338.19 | 1H NMR (400 MHz, DMSO-d₆) δ 10.88 (s, 1H), 8.38 (d, J = 2.6 Hz, 1H), 7.77 (d, J = 8.0 Hz, 2H), 7.49 (t, J = 7.8 Hz, 2H), 7.28 (d, J = 7.4 Hz, 1H), 7.05-6.91 (m, 2H), 6.85 (dd, J = 7.5, 1.6 Hz, 1H), 6.76 (d, J = 2.5 Hz, 1H), |
| | | | | | | 3.80 (s, 3H), 3.70 (s, 3H), |
| | | | | | | 3.67 (d, J = 2.3 Hz, 2H). |
| 8 | 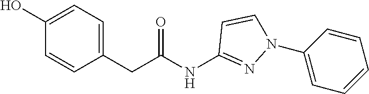 [SMILES: O=C(Cc1ccc(O)cc1)Nc1ccn(-c2ccccc2)n1] | A | 48 | RND | 294.10 | 1H NMR (400 MHz, DMSO-d₆) δ 10.87 (s, 1H), 9.33 (d, J = 3.2 Hz, 1H), 8.38 (d, J = 2.6 Hz, 1H), 7.75 (d, J = 8.0 Hz, 2H), 7.48 (t, J = 7.8 Hz, 2H), 7.28 (d, J = 7.3 Hz, 1H), |
| | | | | | | 7.13 (d, J = 8.1 Hz, 2H), |
| | | | | | | 6.75 (d, J = 2.5 Hz, 1H), |
| | | | | | | 6.71 (d, J = 8.0 Hz, 2H), |
| | | | | | | 3.51 (s, 2H). |
| 9 | 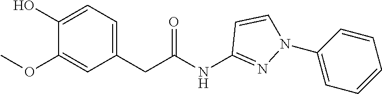 [SMILES: COc1cc(CC(=O)Nc2ccn(-c3ccccc3)n2)ccc1O] | A | 20 | >5 | 324.11 | 1H NMR (400 MHz, DMSO-d₆) δ 10.86 (s, 1H), 8.88 (d, J = 2.3 Hz, 1H), 8.38 (d, J = 2.4 Hz, 1H), 7.76 (d, J = 8.0 Hz, 2H), 7.48 (t, J = 7.8 Hz, 2H), 7.28 (d, J = 7.3 Hz, 1H), |
| | | | | | | 6.92 (s, 1H), 6.76 (d, J = |
| | | | | | | 2.4 Hz, 1H), 6.72 (d, J = 2.7 |
| | | | | | | Hz, 2H), 3.76 (s, 3H), 3.52 |
| | | | | | | (s, 2H). |
| 10 | 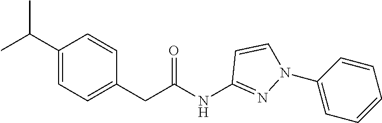 [SMILES: CC(C)c1ccc(CC(=O)Nc2ccn(-c3ccccc3)n2)cc1] | A | 61 | 4.4 | 320.21 | 1H NMR (400 MHz, DMSO-d₆) δ 10.94 (s, 1H), 8.38 (d, J = 2.6 Hz, 1H), 7.76 (d, J = 8.0 Hz, 2H), 7.48 (t, J = 7.8 Hz, 2H), 7.33-7.22 (m, 3H), 7.19 (d, J = 7.9 Hz, 2H), 6.75 (d, J = 2.5 Hz, 1H), 3.60 (s, |
| | | | | | | 2H), 1.18 (d, J = 6.9 Hz, |
| | | | | | | 6H). |
| 11 | 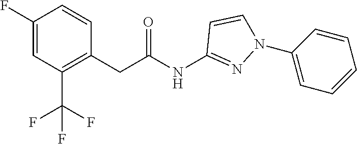 [SMILES: O=C(Cc1ccc(F)cc1C(F)(F)F)Nc1ccn(-c2ccccc2)n1] | A | 71 | RND | 364.16 | 1H NMR (400 MHz, DMSO-d₆) δ 11.02 (s, 1H), 8.39 (d, J = 2.6 Hz, 1H), 7.77 (d, J = 8.0 Hz, 2H), 7.63-7.53 (m, 3H), 7.49 (t, J = 7.9 Hz, 2H), 7.28 (t, J = 7.4 Hz, 1H), 6.72 (d, J = 2.5 Hz, 1H), 3.95 (s, 2H). |
| 12 | 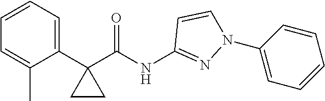 [SMILES: Cc1ccccc1C1(C(=O)Nc2ccn(-c3ccccc3)n2)CC1] | E | 16 | 0.13 | 318.50 | 1H NMR (300 MHz, CDCl₃) δ 7.79 (d, J = 2.5 Hz, 1H), 7.67 (s, 1H), 7.62-7.50 (m, 2H), 7.41 (m, 3H), 7.36- 7.20 (m, 5H), 6.96 (d, J = 2.5 Hz, 1H), 2.41 (s, 3H), 1.83 (m, 2H), 1.16 (m, 2H). |
| 13 | 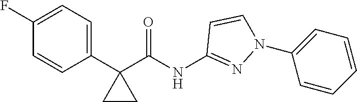 [SMILES: O=C(Nc1ccn(-c2ccccc2)n1)C1(c2ccc(F)cc2)CC1] | E | 68 | 0.082 | 322.46 | 1H NMR (300 MHz, CDCl₃) δ 7.80 (d, J = 2.5 Hz, 1H), 7.66 (s, 1H), 7.62-7.47 (m, 4H), 7.47-7.35 (m, 2H), 7.24 (m, 1H), 7.21- 7.01 (m, 2H), 6.95 (d, J = 2.5 Hz, 1H), 1.83-1.70 (m, |
| | | | | | | 2H), 1.18 (m, 2H). |
| 14 | 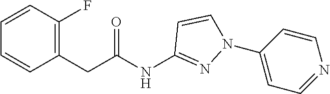 [SMILES: O=C(Cc1ccccc1F)Nc1ccn(-c2ccncc2)n1] | E | 31 | 0.58 | 297.44 | 1H NMR (300 MHz, DMSO-d₆) δ 11.14 (s, 1H), 8.63 (m, 3H), 7.77 (d, J = 6.1 Hz, 2H), 7.54-7.27 (m, 2H), 7.17 (m, 2H), 6.86 (d, J = 2.3 Hz, 1H), 3.77 (s, 2H). |
| 15 | 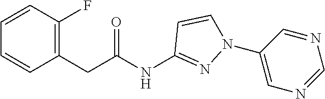 [SMILES: O=C(Cc1ccccc1F)Nc1ccn(-c2cncnc2)n1] | E | 30 | 1.0 | 298.48 | 1H NMR (400 MHz, DMSO-d₆) δ 11.16 (s, 1H), 9.24 (s, 2H), 9.10 (s, 1H), 8.58 (d, J = 2.6 Hz, 1H), 7.39 (td, J = 7.6, 1.7 Hz, 1H), 7.31 (ddd, J = 7.3, 5.6, 1.9 Hz, 1H), 7.23-7.13 (m, |
| | | | | | | 2H), 6.88 (d, J = 2.6 Hz, |
| | | | | | | 1H), 3.78 (s, 2H). |
| 16 | 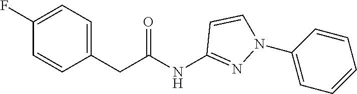 [SMILES: O=C(Cc1ccc(F)cc1)Nc1ccn(-c2ccccc2)n1] | E | 77 | 0.33 | 296.15 | 1H NMR (400 MHz, DMSO-d₆) δ 10.98 (s, 1H), 8.40 (d, J = 2.6 Hz, 1H), 7.77 (dd, J = 8.6, 0.9 Hz, 2H), 7.53-7.45 (m, 2H), 7.41-7.34 (m, 2H), 7.27 (t, J = 7.4 Hz, 1H), 7.15 |
| | | | | | | (ddd, J = 9.7, 5.9, 2.6 Hz, |
| | | | | | | 2H), 6.76 (d, J = 2.5 Hz, |
| | | | | | | 1H), 3.66 (s, 2H). |
| 17 | 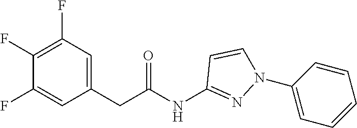 [SMILES: O=C(Cc1cc(F)c(F)c(F)c1)Nc1ccn(-c2ccccc2)n1] | E | 56 | >5 | 332.10 | 1H NMR (300 MHz, CDCl₃) δ 7.93 (s, 1H), 7.84 (d, J = 2.5 Hz, 1H), 7.60 (d, J = 7.7 Hz, 2H), 7.46 (t, J = 7.9 Hz, 2H), 7.36-7.28 (m, 1H), 7.06-6.83 (m, 3H), 5.32 (s, 2H). |
| 18 | 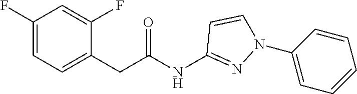 [SMILES: O=C(Cc1ccc(F)cc1F)Nc1ccn(-c2ccccc2)n1] | E | 58 | 0.26 | 314.13 | 1H NMR (300 MHz, CDCl₃) δ 7.94 (s, 1H), 7.83 (d, J = 2.5 Hz, 1H), 7.66-7.55 (m, 2H), 7.45 (m, 2H), 7.41- 7.25 (m, 3H), 7.04-6.80 (m, 3H), 5.32 (s, 2H). |
| 19 | 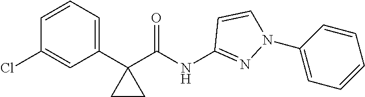 [SMILES: O=C(Nc1ccn(-c2ccccc2)n1)C1(c2cccc(Cl)c2)CC1] | E | 34 | 0.25 | 338.48 | 1H NMR (300 MHz, CDCl₃) δ 7.90 (s, 1H), 7.78 (d, J = 2.6 Hz, 1H), 7.60-7.49 (m, 3H), 7.49-7.36 (m, 5H), 7.34-7.28 (m, 1H), 6.96 (d, J = 2.6 Hz, 1H), 1.77 (m, 2H), 1.31-1.09 (m, |
| | | | | | | 2H). |
| 20 | 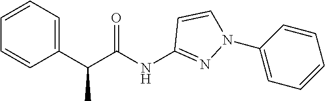 [SMILES: C[C@H](C(=O)Nc1ccn(-c2ccccc2)n1)c1ccccc1] | E | 58 | 0.026 | 292.49 | 1H NMR (300 MHz, CDCl₃) δ 8.29 (s, 1H), 7.79 (d, J = 2.6 Hz, 1H), 7.53 (m, 2H), 7.50-7.36 (m, 6H), 7.36- 7.28 (m, 2H), 7.02 (d, J = 2.6 Hz, 1H), 3.78 (q, J = 7.1 Hz, 1H), 1.64 (d, J = 7.1 Hz, 3H). |
| 21 | 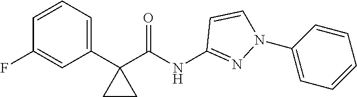 [SMILES: O=C(Nc1ccn(-c2ccccc2)n1)C1(c2cccc(F)c2)CC1] | E | 48 | 0.15 | 321.51 | 1H NMR (300 MHz, CDCl₃) δ 7.87 (s, 1H), 7.78 (d, J = 2.6 Hz, 1H), 7.57-7.49 (m, 2H), 7.49-7.37 (m, 3H), 7.37-7.17 (m, 3H), 7.17- 7.03 (m, 1H), 6.95 (d, J = 2.6 Hz, 1H), 1.86-1.70 (m, |
| | | | | | | 2H), 1.22 (q, J = 3.9 Hz, |
| | | | | | | 2H). |
| 22 | 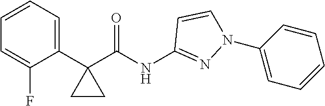 [SMILES: O=C(Nc1ccn(-c2ccccc2)n1)C1(c2ccccc2F)CC1] | E | 17 | 0.012 | 322.51 | 1H NMR (300 MHz, CDCl₃) δ 7.97 (s, 1H), 7.78 (d, J = 2.6 Hz, 1H), 7.44 (m, 6H), 7.32-7.07 (m, 3H), 6.99 (d, J = 2.6 Hz, 1H), 1.91- 1.70 (m, 2H), 1.29-1.11 (m, 2H). |
| 23 | 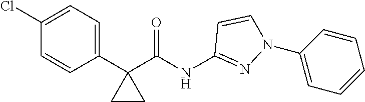 [SMILES: O=C(Nc1ccn(-c2ccccc2)n1)C1(c2ccc(Cl)cc2)CC1] | E | 28 | 0.83 | 338.44 | 1H NMR (300 MHz, CDCl₃) δ 7.79 (d, J = 2.5 Hz, 1H), 7.66 (s, 1H), 7.54 (d, J = 7.7 Hz, 2H), 7.43 (m, 6H), 7.24 (m, 1H), 6.94 (d, J = 2.5 Hz, 1H), 1.82-1.70 (m, 2H), 1.18 (q, J = 3.9 Hz, 2H). |
| 24 | 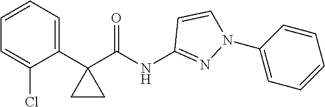 [SMILES: O=C(Nc1ccn(-c2ccccc2)n1)C1(c2ccccc2Cl)CC1] | E | 16 | 0.041 | 338.44 | 1H NMR (300 MHz, CDCl₃) δ 7.81 (s, 1H), 7.78 (d, J = 2.6 Hz, 1H), 7.59-7.46 (m, 4H), 7.48-7.34 (m, 4H), 7.29-7.23 (m, 1H), 7.00 (d, J = 2.6 Hz, 1H), 1.89 (q, J = 4.0 Hz, 2H), 1.24 (q, J = 4.0 Hz, 2H). |
| 25 | 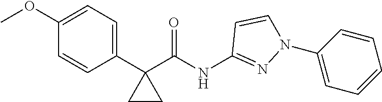 [SMILES: COc1ccc(C2(C(=O)Nc3ccn(-c4ccccc4)n3)CC2)cc1] | E | 36 | 3.6 | 334.52 | 1H NMR (300 MHz, CDCl₃) δ 7.93 (s, 1H), 7.78 (d, J = 2.6 Hz, 1H), 7.62-7.47 (m, 2H), 7.42 (m, 4H), 7.31- 7.23 (m, 1H), 7.02-6.91 (m, 3H), 3.87 (s, 3H), 1.73 (q, J = 3.8 Hz, 2H), 1.19 (q, J = 3.8 Hz, 2H). |
| 26 | 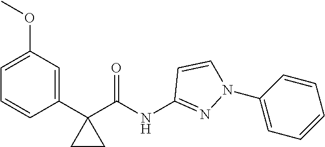 [SMILES: COc1cccc(C2(C(=O)Nc3ccn(-c4ccccc4)n3)CC2)c1] | E | 40 | 0.27 | 334.52 | 1H NMR (300 MHz, CDCl₃) δ 7.95 (s, 1H), 7.78 (d, J = 2.6 Hz, 1H), 7.58-7.49 (m, 2H), 7.49-7.32 (m, 3H), 7.32-7.26 (m, 1H), 7.17- 7.08 (m, 1H), 7.08-7.01 (m, 1H), 6.99-6.86 (m, 2H), 3.86 (s, 3H), 1.82- 1.62 (m, 2H), 1.29-1.10 (m, 2H). |
| 27 | 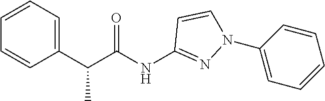 [SMILES: C[C@@H](C(=O)Nc1ccn(-c2ccccc2)n1)c1ccccc1] | E | 54 | 0.21 | 292.54 | 1H NMR (300 MHz, CDCl₃) δ 7.81 (d, J = 2.5 Hz, 1H), 7.79 (s, 1H), 7.56 (m, 2H), 7.51-7.21 (m, 8H), 6.99 (d, J = 2.5 Hz, 1H), 3.76 (q, J = 7.2 Hz, 1H), 1.64 (d, J = 7.2 Hz, 3H). |
| 28 | 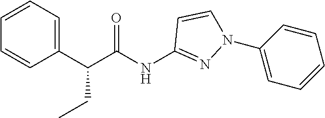 [SMILES: CC[C@@H](C(=O)Nc1ccn(-c2ccccc2)n1)c1ccccc1] | H | 24 | 0.58 | 306.49 | 1H NMR (300 MHz, CDCl₃) δ 8.30 (s, 1H), 7.79 (m, 1H), 7.64-7.20 (m, 10H), 7.01 (m, 1H), 3.45 (m, 1H), 2.46-2.14 (m, 1H), 2.00- 1.75 (m, 1H), 0.96 (m, 3H). |
| 29 | 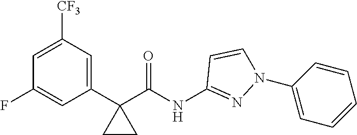 [SMILES: O=C(Nc1ccn(-c2ccccc2)n1)C1(c2cc(F)cc(C(F)(F)F)c2)CC1] | H | 58 | 1.9 | 390.46 | 1H NMR (300 MHz, CDCl₃) δ 7.81 (d, J = 2.5 Hz, 1H), 7.67-7.31 (m, 7H), 7.30- 7.23 (m, 1H), 6.95 (d, J = 2.6 Hz, 1H), 1.83 (q, J = 4.0 Hz, 2H), 1.23 (q, J = 4.0 Hz, 2H). |
| 30 | 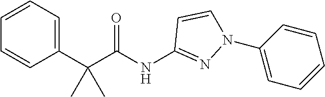 [SMILES: CC(C)(C(=O)Nc1ccn(-c2ccccc2)n1)c1ccccc1] | H | 56 | 0.32 | 306.49 | 1H NMR (300 MHz, CDCl₃) δ 7.80 (d, J = 2.6 Hz, 1H), 7.78 (s, 1H), 7.53 (m, 2H), 7.51-7.38 (m, 6H), 7.36- 7.22 (m, 2H), 7.03 (d, J = 2.6 Hz, 1H), 1.70 (s, 6H). |
| 31 | 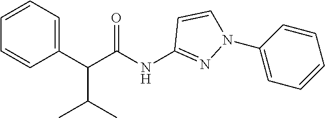 [SMILES: CC(C)C(C(=O)Nc1ccn(-c2ccccc2)n1)c1ccccc1] | H | 64 | >2 | 320.53 | 1H NMR (300 MHz, CDCl₃) δ 9.30 (s, 1H), 7.76 (d, J = 2.6 Hz, 1H), 7.49 (m, 2H), 7.48-7.39 (m, 3H), 7.39- 7.26 (m, 5H), 7.06 (d, J = 2.6 Hz, 1H), 3.08 (m, 1H), 2.55 (m, 1H), 1.13 (d, J = 6.5 Hz, 3H), 0.78 (d, J = 6.7 Hz, 3H). |
| 32 | 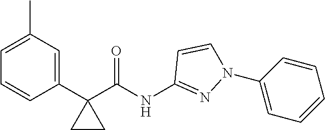 [SMILES: Cc1cccc(C2(C(=O)Nc3ccn(-c4ccccc4)n3)CC2)c1] | H | 41 | 0.65 | 318.5 | 1H NMR (300 MHz, CDCl₃) δ 7.78 (m, 2H), 7.59-7.48 (m, 2H), 7.42 (m, 2H), 7.33 (m, 2H), 7.31-7.14 (m, 3H), 6.97 (d, J = 2.5 Hz, 1H), 2.41 (s, 3H), 1.83- 1.66 (q, J = 3.8 Hz, 2H), 1.20 (q, J = 3.8 Hz, 2H). |
| 33 | 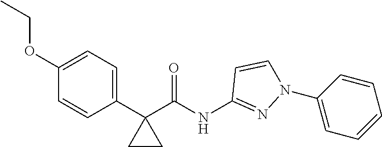 [SMILES: CCOc1ccc(C2(C(=O)Nc3ccn(-c4ccccc4)n3)CC2)cc1] | H | 19 | 0.99 | 348.47 | 1H NMR (300 MHz, CDCl₃) δ 7.85-7.70 (m, 2H), 7.54 (m, 2H), 7.41 (m, 4H), 7.30- 7.20 (m, 1H), 7.03-6.87 (m, 3H), 4.10 (q, J = 7.0 Hz, 2H), 1.72 (q, J = 3.7 Hz, 2H), 1.47 (t, J = 7.0 Hz, 3H), 1.16 (q, J = 3.7 Hz, 2H). |
| 34 | 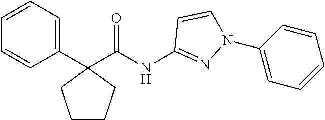 [SMILES: O=C(Nc1ccn(-c2ccccc2)n1)C1(c2ccccc2)CCCC1] | H | 41 | 2.1 | 332.23 | 1H NMR (300 MHz, CDCl₃) δ 8.08 (s, 1H), 7.77 (d, J = 2.5 Hz, 1H), 7.61-7.36 (m, 9H), 7.35-7.27 (m, 1H), 7.01 (d, J = 2.6 Hz, 1H), 2.63 (m, 2H), 2.26-2.07 (m, 2H), 2.01-1.55 (m, 4H). |
| 35 | 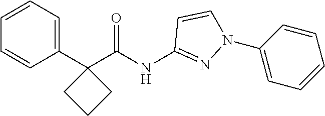 [SMILES: O=C(Nc1ccn(-c2ccccc2)n1)C1(c2ccccc2)CCC1] | H | 26 | 0.33 | 318.46 | 1H NMR (300 MHz, CDCl₃) δ 7.79 (d, J = 2.6 Hz, 1H), 7.75 (s, 1H), 7.52 (m, 2H), 7.43 (m, 6H), 7.36-7.20 (m, 2H), 7.02 (d, J = 2.5 Hz, 1H), 3.02-2.89 (m, 2H), 2.66-2.52 (m, 2H), 2.30-2.12 (m, 1H), 2.05- |
| | | | | | | 1.84 (m, 1H). |
| 36 | 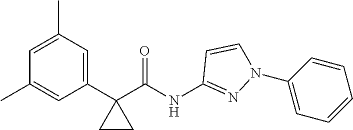 [SMILES: Cc1cc(C)cc(C2(C(=O)Nc3ccn(-c4ccccc4)n3)CC2)c1] | H | 44 | >5 | 332.45 | 1H NMR (300 MHz, CDCl₃) δ 7.84 (s, 1H), 7.78 (d, J = 2.6 Hz, 1H), 7.58-7.49 (m, 2H), 7.48-7.35 (m, 2H), 7.30-7.20 (m, 1H), 7.11 (s, 2H), 7.01 (s, 1H), 6.97 (d, J = 2.5 Hz, 1H), 2.36 (s, 6H), 1.77-1.64 (m, 2H), 1.25-1.07 (m, 2H). |
| 37 | 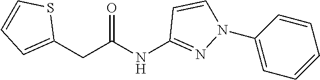 [SMILES: O=C(Cc1cccs1)Nc1ccn(-c2ccccc2)n1] | A | 65 | 0.064 | 284.08 | 1H NMR (400 MHz, DMSO-d₆) δ 11.00 (s, 1H), 8.42 (d, J = 2.6 Hz, 1H), 7.80-7.74 (m, 2H), 7.52- 7.46 (m, 2H), 7.40 (dd, J = 4.5, 1.9 Hz, 1H), 7.28 (t, J = |
| | | | | | | 7.4 Hz, 1H), 7.03-6.95 (m, |
| | | | | | | 2H), 6.78 (d, J = 2.5 Hz, |
| | | | | | | 1H), 3.90 (s, 2H). |
| 38 | 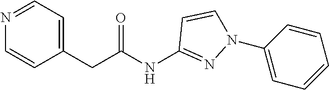 [SMILES: O=C(Cc1ccncc1)Nc1ccn(-c2ccccc2)n1] | A | 89 | >5 | 279.2 | 1H NMR (400 MHz, DMSO-d₆) δ 11.21 (s, 1H), 8.82 (d, J = 5.8 Hz, 2H), 8.41 (d, J = 2.5 Hz, 1H), 7.93 (d, J = 5.6 Hz, 2H), 7.76 (d, J = 8.4 Hz, 2H), |
| | | | | | | 7.49 (t, J = 7.6 Hz, 2H), |
| | | | | | | 7.29 (d, J = 7.9 Hz, 1H), |
| | | | | | | 6.75 (d, J = 2.5 Hz, 1H), |
| | | | | | | 4.05 (s, 2H). |
| 39 | 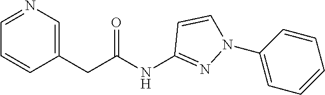 [SMILES: O=C(Cc1cccnc1)Nc1ccn(-c2ccccc2)n1] | A | 11 | >5 | 279.13 | 1H NMR (400 MHz, DMSO-d₆) δ 11.05 (s, 1H), 8.53 (d, J = 2.2 Hz, 1H), 8.46 (dd, J = 4.8, 1.6 Hz, 1H), 8.37 (d, J = 2.6 Hz, 1H), 7.76 (t, J = 6.9 Hz, 3H), 7.48 (t, J = 7.8 Hz, |
| | | | | | | 2H), 7.38 (dd, J = 7.8, 4.9 |
| | | | | | | Hz, 1H), 7.27 (t, J = 7.4 Hz, |
| | | | | | | 1H), 6.75 (d, J = 2.5 Hz, |
| | | | | | | 1H), 3.72 (s, 2H). |
| 40 | 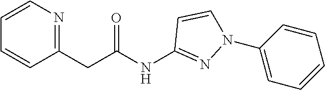 [SMILES: O=C(Cc1ccccn1)Nc1ccn(-c2ccccc2)n1] | A | 41 | 1.5 | 279.13 | 1H NMR (400 MHz, DMSO-d₆) δ 10.99 (s, 1H), 8.49 (d, J = 4.8 Hz, 1H), 8.38 (d, J = 2.6 Hz, 1H), 7.81-7.71 (m, 3H), 7.49 (t, J = 7.8 Hz, 2H), 7.39 (d, |
| | | | | | | J = 7.8 Hz, 1H), 7.28 (t, J = |
| | | | | | | 6.3 Hz, 2H), 6.76 (d, J = 2.6 |
| | | | | | | Hz, 1H), 3.87 (s, 2H). |
| 41 | 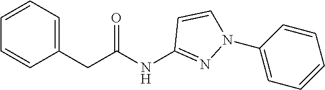 [SMILES: O=C(Cc1ccccc1)Nc1ccn(-c2ccccc2)n1] | A | 51 | 0.073 | 278.14 | 1H NMR (400 MHz, DMSO-d₆) δ 10.97 (s, 1H), 8.37 (d, J = 2.6 Hz, 1H), 7.75 (d, J = 8.0 Hz, 2H), 7.48 (t, J = 7.8 Hz, 2H), 7.40-7.18 (m, 6H), A6.75 |
| | | | | | | (d, J = 2.5 Hz, 1H), 3.65 (s, |
| | | | | | | 2H). |
| 42 | 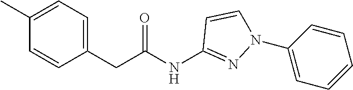 [SMILES: Cc1ccc(CC(=O)Nc2ccn(-c3ccccc3)n2)cc1] | A | 57 | 0.71 | 292.18 | 1H NMR (400 MHz, DMSO-d₆) δ 10.93 (s, 1H), 8.39 (d, J = 2.6 Hz, 1H), 7.76 (dd, J = 8.6, 0.9 Hz, 2H), 7.52-7.45 (m, 2H), 7.30-7.20 (m, 3H), 7.15- |
| | | | | | | 7.11 (m, 2H), 6.76 (d, J = |
| | | | | | | 2.5 Hz, 1H), 3.60 (d, J = 7.8 |
| | | | | | | Hz, 2H), 2.28 (s, 3H). |
| 43 | 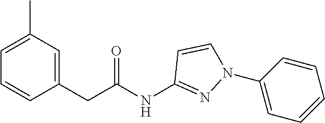 [SMILES: Cc1cccc(CC(=O)Nc2ccn(-c3ccccc3)n2)c1] | A | 38 | 1.9 | 292.14 | 1H NMR (400 MHz, DMSO-d₆) δ 10.94 (s, 1H), 8.37 (d, J = 2.6 Hz, 1H), 7.74 (d, J = 8.1 Hz, 2H), 7.47 (t, J = 7.8 Hz, 2H), 7.26 (t, J = 7.4 Hz, 1H), 7.20 (t, J = 7.5 Hz, 1H), 7.17-7.09 (m, 2H), 7.05 |
| | | | | | | (d, J = 7.5 Hz, 1H), 6.75 (d, |
| | | | | | | J = 2.5 Hz, 1H), 3.60 (s, |
| | | | | | | 2H), 2.28 (s, 3H). |
| 44 | 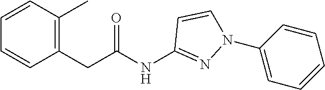 [SMILES: Cc1ccccc1CC(=O)Nc1ccn(-c2ccccc2)n1] | A | 8 | 0.24 | 292.15 | 1H NMR (400 MHz, DMSO-d₆) δ 10.95 (s, 1H), 8.40 (d, J = 2.6 Hz, 1H), 7.82-7.70 (m, 2H), 7.55- 7.42 (m, 2H), 7.34-7.22 (m, 2H), 7.22-7.09 (m, |
| | | | | | | 3H), 6.76 (d, J = 2.5 Hz, |
| | | | | | | 1H), 3.70 (s, 2H), 2.30 (s, |
| | | | | | | 3H). |
| 45 | 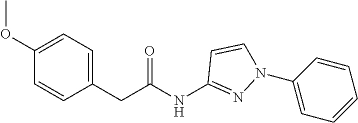 [SMILES: COc1ccc(CC(=O)Nc2ccn(-c3ccccc3)n2)cc1] | A | 31 | >5 | 308.14 | 1H NMR (400 MHz, DMSO-d₆) δ 10.91 (s, 1H), 8.37 (d, J = 2.6 Hz, 1H), 7.81-7.71 (m, 2H), 7.48 (t, J = 7.8 Hz, 2H), 7.32- 7.19 (m, 3H), 6.89 (d, J = 8.3 Hz, 2H), 6.75 (d, J = 2.5 Hz, 1H), 3.72 (s, 3H), 3.57 (s, 2H). |
| 46 | 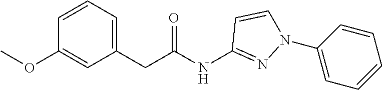 [SMILES: COc1cccc(CC(=O)Nc2ccn(-c3ccccc3)n2)c1] | A | 34 | >5 | 308.11 | 1H NMR (400 MHz, DMSO-d₆) δ 10.94 (s, 1H), 8.37 (d, J = 2.6 Hz, 1H), 7.75 (d, J = 8.1 Hz, 2H), 7.47 (t, J = 7.8 Hz, 2H), 7.24 (dt, J = 14.9, 7.6 Hz, 2H), 6.96-6.84 (m, 2H), |
| | | | | | | 6.81 (dd, J = 8.3, 2.4 Hz, |
| | | | | | | 1H), 6.75 (d, J = 2.5 Hz, |
| | | | | | | 1H), 3.73 (s, 3H), 3.61 (s, |
| | | | | | | 2H). |
| 47 | 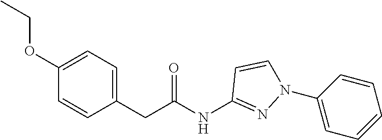 [SMILES: CCOc1ccc(CC(=O)Nc2ccn(-c3ccccc3)n2)cc1] | A | 62 | >5 | 322.19 | 1H NMR (400 MHz, DMSO-d₆) δ 10.91 (s, 1H), 8.38 (d, J = 2.6 Hz, 1H), 7.76 (d, J = 7.9 Hz, 2H), 7.48 (t, J = 7.8 Hz, 2H), 7.33-7.19 (m, 3H), 6.87 (d, J = 8.5 Hz, 2H), 6.75 (d, J = 2.6 Hz, 1H), 3.99 (q, J = 6.9 Hz, 2H), 3.57 (s, 2H), 1.31 (t, J = 6.9 Hz, 3H). |
| 48 | 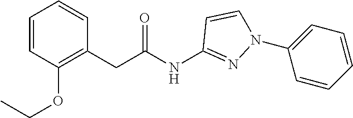 [SMILES: CCOc1ccccc1CC(=O)Nc1ccn(-c2ccccc2)n1] | A | 9 | RND | 322.15 | 1H NMR (400 MHz, DMSO-d₆) δ 10.77 (s, 1H), 8.38 (d, J = 2.6 Hz, 1H), 7.77 (d, J = 8.0 Hz, 2H), 7.49 (t, J = 7.8 Hz, 2H), 7.32-7.16 (m, 3H), 6.95 (d, J = 8.0 Hz, 1H), 6.89 (t, J = 7.4 Hz, 1H), 6.76 (d, J = |
| | | | | | | 2.5 Hz, 1H), 3.99 (q, J = 6.9 |
| | | | | | | Hz, 2H), 3.64 (s, 2H), 1.27 |
| | | | | | | (t, J = 6.9 Hz, 3H). |
| 49 | 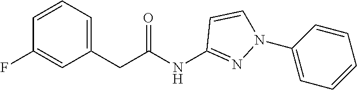 [SMILES: O=C(Cc1cccc(F)c1)Nc1ccn(-c2ccccc2)n1] | A | 44 | 1.9 | 296.14 | 1H NMR (400 MHz, DMSO-d₆) δ 11.00 (s, 1H), 8.38 (d, J = 2.6 Hz, 1H), 7.76 (d, J = 8.0 Hz, 2H), 7.48 (t, J = 7.8 Hz, 2H), 7.37 (q, J = 7.4 Hz, 1H), 7.27 (t, J = 7.4 Hz, 1H), |
| | | | | | | 7.17 (d, J = 8.3 Hz, 2H), |
| | | | | | | 7.14-7.03 (m, 1H), 6.76 |
| | | | | | | (d, J = 2.5 Hz, 1H), 3.69 (s, |
| | | | | | | 2H). |
| 50 | 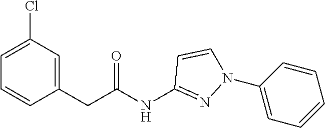 [SMILES: O=C(Cc1cccc(Cl)c1)Nc1ccn(-c2ccccc2)n1] | A | 83 | >5 | 312.21 | 1H NMR (400 MHz, DMSO-d₆) δ 11.01 (s, 1H), 8.39 (d, J = 2.6 Hz, 1H), 7.76 (d, J = 8.0 Hz, 2H), 7.49 (t, J = 7.8 Hz, 2H), 7.44-7.23 (m, 5H), 6.75 (d, J = 2.5 Hz, 1H), 3.69 (s, 2H). |
| 51 | 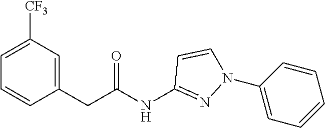 [SMILES: O=C(Cc1cccc(C(F)(F)F)c1)Nc1ccn(-c2ccccc2)n1] | A | 57 | >2 | 346.15 | 1H NMR (400 MHz, DMSO-d₆) δ 11.06 (s, 1H), 8.41 (d, J = 2.6 Hz, 1H), 7.80-7.75 (m, 2H), 7.72 (s, 1H), 7.64 (t, J = 6.7 Hz, 2H), 7.61-7.55 (m, 1H), 7.49 (dd, J = 10.8, 5.2 Hz, 2H), 7.28 (t, J = 7.4 Hz, 1H), 6.77 (d, J = 2.5 Hz, |
| | | | | | | 1H), 3.80 (d, J = 6.4 Hz, |
| | | | | | | 2H). |
| 52 | 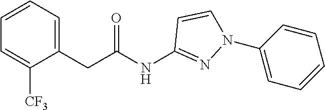 [SMILES: O=C(Cc1ccccc1C(F)(F)F)Nc1ccn(-c2ccccc2)n1] | D | 47 | 1.1 | 346.18 | 1H NMR (400 MHz, DMSO-d₆) δ 11.02 (s, 1H), 8.40 (d, J = 2.6 Hz, 1H), 7.79-7.74 (m, 2H), 7.74- 7.68 (m, 1H), 7.65 (m, 1H), 7.56-7.45 (m, 4H), 7.28 (m, 1H), 6.73 (d, J = 2.6 Hz, 1H), 3.95 (s, 2H). |
| 53 | 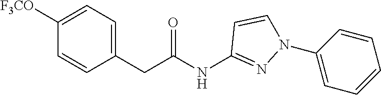 [SMILES: O=C(Cc1ccc(OC(F)(F)F)cc1)Nc1ccn(-c2ccccc2)n1] | A | 53 | >5 | 362.12 | 1H NMR (400 MHz, DMSO-d₆) δ 11.03 (s, 1H), 8.40 (d, J = 2.6 Hz, 1H), 7.77 (dd, J = 8.6, 0.9 Hz, 2H), 7.52-7.43 (m, 4H), 7.35-7.24 (m, 3H), 6.77 (d, J = 2.5 Hz, 1H), 3.72 (s, |
| | | | | | | 2H). |
| 54 | 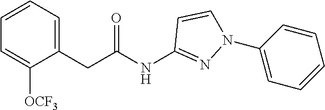 [SMILES: O=C(Cc1ccccc1OC(F)(F)F)Nc1ccn(-c2ccccc2)n1] | A | 53 | >5 | 362.12 | 1H NMR (400 MHz, DMSO-d₆) δ 11.04 (s, 1H), 8.41 (d, J = 2.6 Hz, 1H), 7.78 (dd, J = 8.6, 0.9 Hz, 2H), 7.53-7.46 (m, 3H), 7.45-7.33 (m, 3H), 7.28 (t, J = 7.4 Hz, 1H), 6.76 (d, J = 2.5 Hz, 1H), 3.82 (s, |
| | | | | | | 2H). |
| 55 | 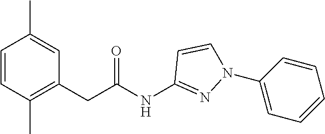 [SMILES: Cc1ccc(C)c(CC(=O)Nc2ccn(-c3ccccc3)n2)c1] | A | 20 | >5 | 306.19 | 1H NMR (400 MHz, DMSO-d₆) δ 10.92 (s, 1H), 8.39 (d, J = 2.6 Hz, 1H), 7.80-7.72 (m, 2H), 7.48 (t, J = 8.0 Hz, 2H), 7.26 (t, J = 7.4 Hz, 1H), 7.08-7.01 (m, 2H), 6.94 (dd, J = 7.7, 1.9 Hz, 1H), 6.75 (d, J = 2.5 Hz, 1H), 3.65 (s, 2H), 2.24 (s, 6H). |
| 56 | 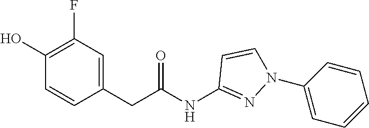 [SMILES: O=C(Cc1ccc(O)c(F)c1)Nc1ccn(-c2ccccc2)n1] | A | 11 | >5 | 312.10 | 1H NMR (400 MHz, DMSO-d₆) δ 10.90 (s, 1H), 9.77 (s, 1H), 8.38 (d, J = 2.6 Hz, 1H), 7.75 (d, J = 8.0 Hz, 2H), 7.47 (t, J = 7.8 Hz, 2H), 7.26 (t, J = 7.4 Hz, 1H), 7.10 (dd, J = 12.4, 1.9 Hz, 1H), 6.96-6.84 (m, |
| | | | | | | 2H), 6.75 (d, J = 2.6 Hz, |
| | | | | | | 1H), 3.54 (s, 2H). |
| 57 | 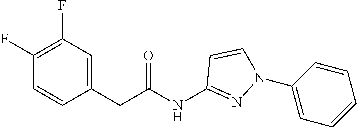 [SMILES: O=C(Cc1ccc(F)c(F)c1)Nc1ccn(-c2ccccc2)n1] | A | 60 | >5 | 314.09 | 1H NMR (400 MHz, DMSO-d₆) δ 10.98 (s, 1H), 8.37 (d, J = 2.6 Hz, 1H), 7.74 (d, J = 8.0 Hz, 2H), 7.47 (t, J = 7.7 Hz, 2H), 7.37 (dt, J = 11.2, 8.3 Hz, 2H), 7.26 (t, J = 7.4 Hz, 1H), 6.74 (d, J = 2.5 Hz, 1H), 3.67 (s, 2H). |
| 58 | 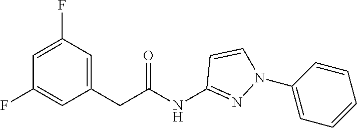 [SMILES: O=C(Cc1cc(F)cc(F)c1)Nc1ccn(-c2ccccc2)n1] | A | 59 | >5 | 314.13 | 1H NMR (400 MHz, DMSO-d₆) δ 11.01 (s, 1H), 8.38 (d, J = 2.6 Hz, 1H), 7.75 (d, J = 7.9 Hz, 2H), 7.48 (t, J = 7.7 Hz, 2H), 7.27 (t, J = 7.4 Hz, 1H), 7.12 (tt, J = 9.5, 2.5 Hz, 1H), 7.05 (h, J = 4.7 Hz, 2H), 6.75 (d, J = 2.5 Hz, |
| | | | | | | 1H), 3.72 (s, 2H). |
| 59 | 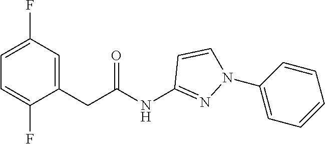 [SMILES: O=C(Cc1cc(F)ccc1F)Nc1ccn(-c2ccccc2)n1] | A | 43 | 0.25 | 314.12 | 1H NMR (400 MHz, DMSO-d₆) δ 11.02 (s, 1H), 8.38 (d, J = 2.6 Hz, 1H), 7.75 (d, J = 8.0 Hz, 2H), 7.48 (t, J = 7.7 Hz, 2H), 7.30-7.19 (m, 3H), 7.15 (tt, J = 8.3, 3.6 Hz, 1H), 6.74 (d, J = 2.5 Hz, 1H), 3.76 (s, 2H). |
| 60 | 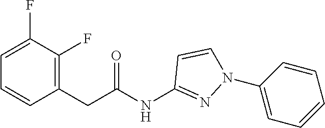 [SMILES: O=C(Cc1cccc(F)c1F)Nc1ccn(-c2ccccc2)n1] | A | 15 | >2 | 314.13 | 1H NMR (400 MHz, DMSO-d₆) δ 11.06 (s, 1H), 8.39 (d, J = 2.6 Hz, 1H), 7.76 (d, J = 8.0 Hz, 2H), 7.48 (t, J = 7.8 Hz, 2H), 7.37-7.24 (m, 2H), 7.23- 7.14 (m, 2H), 6.74 (d, J = 2.5 Hz, 1H), 3.82 (s, 2H). |
| 61 | 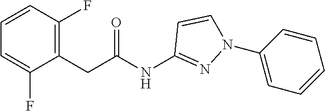 [SMILES: O=C(Cc1c(F)cccc1F)Nc1ccn(-c2ccccc2)n1] | A | 61 | 0.065 | 314.12 | 1H NMR (400 MHz, DMSO-d₆) δ 11.10 (s, 1H), 8.41 (d, J = 2.5 Hz, 1H), 7.78 (d, J = 7.7 Hz, 2H), 7.50 (dd, J = 10.8, 5.2 Hz, 2H), 7.43-7.35 (m, 1H), 7.28 (t, J = 7.4 Hz, 1H), 7.15-7.07 (m, 2H), 6.74 |
| | | | | | | (d, J = 2.5 Hz, 1H), 3.80 (s, |
| | | | | | | 2H). |
| 62 | 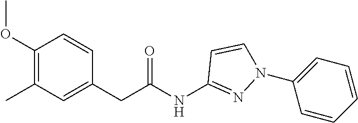 [SMILES: COc1ccc(CC(=O)Nc2ccn(-c3ccccc3)n2)cc1C] | A | 54 | 3.7 | 322.19 | 1H NMR (400 MHz, DMSO-d₆) δ 10.87 (s, 1H), 8.36 (d, J = 2.6 Hz, 1H), 7.74 (d, J = 8.0 Hz, 2H), 7.47 (t, J = 7.8 Hz, 2H), 7.26 (t, J = 7.4 Hz, 1H), 7.16-7.05 (m, 2H), 6.86 (d, J = 8.2 Hz, 1H), 6.74 (d, |
| | | | | | | J = 2.5 Hz, 1H), 3.74 (s, |
| | | | | | | 3H), 2.12 (s, 3H). |
| 63 | 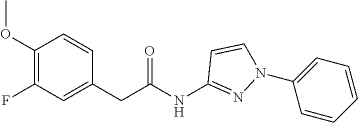 [SMILES: COc1ccc(CC(=O)Nc2ccn(-c3ccccc3)n2)cc1F] | A | 53 | >5 | 326.16 | 1H NMR (400 MHz, DMSO-d₆) δ 10.93 (s, 1H), 8.37 (d, J = 2.6 Hz, 1H), 7.75 (d, J = 8.0 Hz, 2H), 7.48 (t, J = 7.7 Hz, 2H), 7.17 (d, J = 12.3 Hz, 1H), 7.10 (d, J = 7.5 Hz, 2H), 3.80 (s, 3H), 3.59 (s, 2H). |
| 64 | 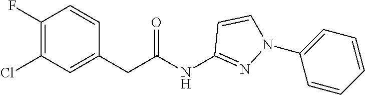 [SMILES: O=C(Cc1ccc(F)c(Cl)c1)Nc1ccn(-c2ccccc2)n1] | A | 31 | >5 | 330.12 | 1H NMR (400 MHz, DMSO-d₆) δ 11.00 (s, 1H), 8.38 (d, J = 2.6 Hz, 1H), 7.75 (d, J = 8.0 Hz, 2H), 7.54 (dd, J = 7.3, 1.9 Hz, 1H), 7.48 (t, J = 7.8 Hz, 2H), 7.40-7.29 (m, 2H), |
| | | | | | | 7.27 (t, J = 7.3 Hz, 1H), |
| | | | | | | 6.75 (d, J = 2.5 Hz, 1H), |
| | | | | | | 3.68 (s, 2H). |
| 65 | 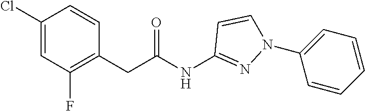 [SMILES: O=C(Cc1ccc(Cl)cc1F)Nc1ccn(-c2ccccc2)n1] | A | 34 | RND | 330.13 | 1H NMR (400 MHz, DMSO-d₆) δ 11.03 (s, 1H), 8.41 (d, J = 2.5 Hz, 1H), 7.77 (d, J = 7.7 Hz, 2H), 7.52-7.38 (m, 4H), 7.31- 7.25 (m, 2H), 6.75 (d, J = 2.5 Hz, 1H), 3.77 (s, 2H). |
| 66 | 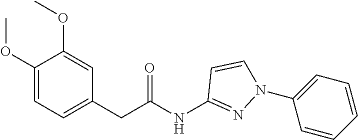 [SMILES: COc1ccc(CC(=O)Nc2ccn(-c3ccccc3)n2)cc1OC] | A | 70 | 2.8 | 338.20 | 1H NMR (400 MHz, DMSO-d₆) δ 10.89 (s, 1H), 8.38 (d, J = 2.5 Hz, 1H), 7.75 (d, J = 8.1 Hz, 2H), 7.48 (m, 2H), 7.27 (m, 1H), 6.96 (s, 1H), 6.88 (m, 2H), 6.76 (d, J = 2.5 Hz, 1H), 3.73 (m, 6H), 3.56 (s, 2H). |
| 67 | 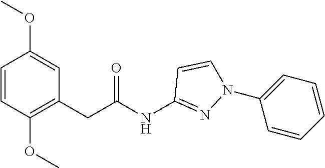 [SMILES: COc1ccc(OC)c(CC(=O)Nc2ccn(-c3ccccc3)n2)c1] | A | 52 | RND | 338.21 | 1H NMR (400 MHz, DMSO-d₆) δ 10.78 (s, 1H), 8.38 (d, J = 2.6 Hz, 1H), 7.76 (d, J = 8.1 Hz, 2H), 7.49 (m, 2H), 7.27 (m, 1H), 6.94-6.70 (m, 4H), 3.70 (m, 6H), 3.65 (s, 2H). |
| 68 | 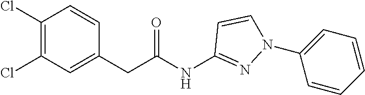 [SMILES: O=C(Cc1ccc(Cl)c(Cl)c1)Nc1ccn(-c2ccccc2)n1] | A | 50 | >5 | 346.12 | 1H NMR (400 MHz, DMSO-d₆) δ 11.02 (s, 1H), 8.38 (d, J = 2.6 Hz, 1H), 7.75 (d, J = 8.1 Hz, 2H), 7.59 (m, 2H), 7.48 (m, 2H), 7.39-7.22 (m, 2H), 6.75 (d, J = 2.6 Hz, 1H), 3.70 (s, |
| | | | | | | 2H). |
| 69 | 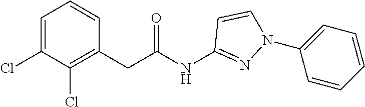 [SMILES: O=C(Cc1cccc(Cl)c1Cl)Nc1ccn(-c2ccccc2)n1] | A | 63 | 5.0 | 347.31 | 1H NMR (400 MHz, DMSO-d₆) δ 11.07 (s, 1H), 8.41 (d, J = 2.6 Hz, 1H), 7.83-7.69 (m, 2H), 7.63- 7.24 (m, 6H), 6.74 (d, J = 2.6 Hz, 1H), 3.94 (s, 2H). |
| 70 | 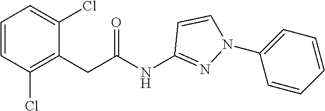 [SMILES: O=C(Cc1c(Cl)cccc1Cl)Nc1ccn(-c2ccccc2)n1] | A | 58 | >2 | 346.18 | 1H NMR (400 MHz, DMSO-d₆) δ 11.11 (s, 1H), 8.39 (d, J = 2.6 Hz, 1H), 7.76 (d, J = 8.0 Hz, 2H), 7.55-7.44 (m, 4H), 7.31 (m, 2H), 6.72 (d, J = 2.6 Hz, 1H), 4.07 (s, 2H). |
| 71 | 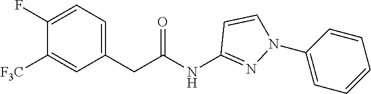 [SMILES: O=C(Cc1ccc(F)c(C(F)(F)F)c1)Nc1ccn(-c2ccccc2)n1] | A | 24 | >5 | 364.09 | 1H NMR (400 MHz, DMSO-d₆) δ 11.04 (s, 1H), 8.40 (d, J = 2.6 Hz, 1H), 7.83-7.65 (m, 4H), 7.49 (m, 3H), 7.27 (m, 1H), 6.75 (d, J = 2.6 Hz, 1H), 3.79 (s, 2H). |
| 72 | 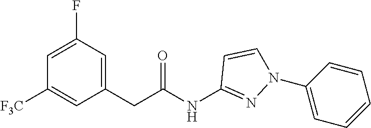 [SMILES: O=C(Cc1cc(F)cc(C(F)(F)F)c1)Nc1ccn(-c2ccccc2)n1] | A | 58 | >5 | 364.16 | 1H NMR (400 MHz, DMSO-d₆) δ 11.07 (s, 1H), 8.41 (d, J = 2.6 Hz, 1H), 7.77 (d, J = 8.0 Hz, 2H), 7.67-7.44 (m, 5H), 7.28 (m, 1H), 6.76 (d, J = 2.6 Hz, 1H), 3.84 (s, 2H). |
| 73 | 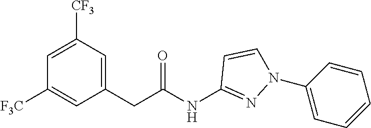 [SMILES: O=C(Cc1cc(C(F)(F)F)cc(C(F)(F)F)c1)Nc1ccn(-c2ccccc2)n1] | A | 97 | >5 | 414.13 | 1H NMR (400 MHz, DMSO-d₆) δ 11.10 (s, 1H), 8.41 (d, J = 2.6 Hz, 1H), 8.04 (m, 3H), 7.82-7.68 (m, 2H), 7.49 (m, 2H), 7.28 (m, 1H), 6.76 (d, J = 2.6 Hz, 1H), 3.96 (s, 2H). |
| 74 | 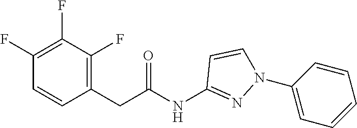 [SMILES: O=C(Cc1ccc(F)c(F)c1F)Nc1ccn(-c2ccccc2)n1] | A | 74 | >5 | 332.17 | 1H NMR (400 MHz, DMSO-d₆) δ 11.07 (s, 1H), 8.40 (d, J = 2.6 Hz, 1H), 7.77 (d, J = 8.1 Hz, 2H), 7.49 (m, 2H), 7.34-7.21 (m, 3H), 6.75 (d, J = 2.6 Hz, 1H), 3.82 (s, 2H). |
| 75 | 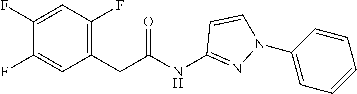 [SMILES: O=C(Cc1cc(F)c(F)cc1F)Nc1ccn(-c2ccccc2)n1] | A | 69 | >2 | 332.08 | 1H NMR (400 MHz, DMSO-d₆) δ 11.03 (s, 1H), 8.38 (d, J = 2.6 Hz, 1H), 7.76 (d, J = 8.0 Hz, 2H), 7.52 (m, 4H), 7.28 (m, 1H), 6.74 (d, J = 2.6 Hz, 1H), 3.75 (s, 2H). |
| 76 | 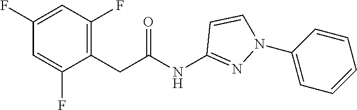 [SMILES: O=C(Cc1c(F)cc(F)cc1F)Nc1ccn(-c2ccccc2)n1] | A | 10 | 0.072 | 332.08 | 1H NMR (400 MHz, DMSO-d₆) δ 11.09 (s, 1H), 8.39 (d, J = 2.6 Hz, 1H), 7.76 (d, J = 8.0 Hz, 2H), 7.48 (t, J = 7.8 Hz, 2H), 7.27 (t, J = 7.4 Hz, 1H), 7.23-7.12 (m, 2H), 6.72 |
| | | | | | | (d, J = 2.5 Hz, 1H), 3.75 (s, |
| | | | | | | 2H). |
| 77 | 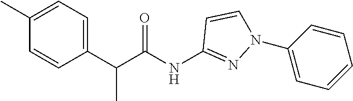 [SMILES: Cc1ccc(C(C)C(=O)Nc2ccn(-c3ccccc3)n2)cc1] | A | 71 | 3.3 | 306.18 | 1H NMR (400 MHz, DMSO-d₆) δ 10.83 (s, 1H), 8.35 (d, J = 2.6 Hz, 1H), 7.72 (d, J = 8.1 Hz, 2H), 7.46 (t, J = 7.8 Hz, 2H), 7.26 (d, J = 7.8 Hz, 3H), 7.12 (d, J = 7.8 Hz, 2H), 6.75 (d, J = 2.5 Hz, 1H), |
| | | | | | | 3.85 (q, J = 6.9 Hz, 1H), |
| | | | | | | 2.25 (s, 3H), 1.37 (d, J = |
| | | | | | | 7.0 Hz, 3H). |
| 78 | 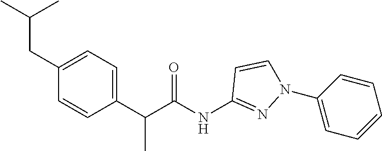 [SMILES: CC(C)Cc1ccc(C(C)C(=O)Nc2ccn(-c3ccccc3)n2)cc1] | A | 53 | >5 | 348.23 | 1H NMR (400 MHz, DMSO-d₆) δ 10.85 (s, 1H), 8.36 (d, J = 2.6 Hz, 1H), 7.73 (d, J = 8.1 Hz, 2H), 7.46 (t, J = 7.7 Hz, 2H), 7.33-7.22 (m, 3H), 7.09 (d, J = 7.8 Hz, 2H), 6.77 (d, J = 2.5 Hz, 1H), 3.86 (q, J = 6.9 Hz, 1H), 2.39 (d, J = 7.1 Hz, 2H), 1.78 (dp, J = 13.5, |
| | | | | | | 6.6 Hz, 1H), 1.38 (d, J = 6.9 |
| | | | | | | Hz, 3H), 0.83 (d, J = 6.6 Hz, |
| | | | | | | 6H). |
| 79 | 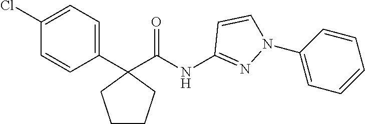 [SMILES: O=C(Nc1ccn(-c2ccccc2)n1)C1(c2ccc(Cl)cc2)CCCC1] | A | 19 | >5 | 366.21 | 1H NMR (400 MHz, DMSO-d₆) δ 10.18 (s, 1H), 8.33 (d, J = 2.6 Hz, 1H), 7.71 (d, J = 8.1 Hz, 2H), 7.42 (p, J = 8.6, 8.2 Hz, 6H), 7.24 (t, J = 7.4 Hz, 1H), 6.75 (d, J = 2.5 Hz, 1H), 2.75-2.59 (m, 2H), |
| | | | | | | 1.88 (dd, J = 13.3, 6.8 Hz, |
| | | | | | | 2H), 1.69-1.54 (m, 4H). |
| 80 | 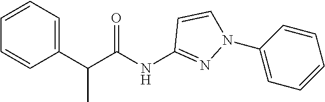 [SMILES: CC(C(=O)Nc1ccn(-c2ccccc2)n1)c1ccccc1] | A | 70 | 0.047 | 292.12 | 1H NMR (400 MHz, DMSO-d₆) δ 10.88 (s, 1H), 8.36 (d, J = 2.6 Hz, 1H), 7.72 (d, J = 8.0 Hz, 2H), 7.46 (t, J = 7.7 Hz, 2H), 7.39 (d, J = 7.7 Hz, 2H), 7.32 (t, J = 7.5 Hz, 2H), |
| | | | | | | 7.24 (q, J = 7.9 Hz, 2H), |
| | | | | | | 6.76 (d, J = 2.5 Hz, 1H), |
| | | | | | | 3.90 (q, J = 7.0 Hz, 1H), |
| | | | | | | 1.40 (d, J = 7.0 Hz, 3H). |
| 81 | 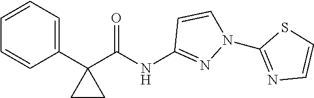 [SMILES: O=C(Nc1ccn(-c2nccs2)n1)C1(c2ccccc2)CC1] | I | 30 | 0.011 | 311.53 | 1H NMR (400 MHz, DMSO-d₆) δ 9.77 (s, 1H), 8.38 (d, J = 2.7 Hz, 1H), 7.59 (d, J = 3.5 Hz, 1H), 7.47 (d, J = 3.5 Hz, 1H), 7.44 (d, J = 1.7 Hz, 1H), 7.38 (t, J = 7.4 Hz, 2H), |
| | | | | | | 7.35-7.27 (m, 2H), 6.85 |
| | | | | | | (d, J = 2.7 Hz, 1H), 1.48 (q, |
| | | | | | | J = 4.3 Hz, 2H), 1.15-1.12 |
| | | | | | | (m, 2H). |
| 82 | 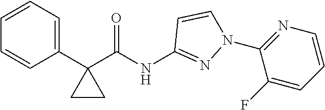 [SMILES: O=C(Nc1ccn(-c2ncccc2F)n1)C1(c2ccccc2)CC1] | I | 27 | 0.19 | 323.54 | 1H NMR (400 MHz, DMSO-d₆) δ 9.44 (s, 1H), 8.41-8.24 (m, 2H), 7.97 (ddd, J = 11.6, 8.3, 1.3 Hz, 1H), 7.59-7.25 (m, 5H), 6.86 (d, J = 2.7 Hz, 1H), 1.63-1.41 (m, 2H), 1.22- 1.02 (m, 2H). |
| 83 | 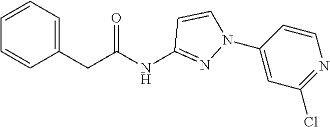 [SMILES: O=C(Cc1ccccc1)Nc1ccn(-c2ccnc(Cl)c2)n1] | I | 46 | 0.60 | 313.51 | 1H NMR (400 MHz, DMSO-d₆) δ 11.06 (s, 1H), 8.59 (d, J = 2.8 Hz, 1H), 8.38 (d, J = 5.6 Hz, 1H), 7.82 (d, J = 1.9 Hz, 1H), 7.71 (dd, J = 5.6, 2.0 Hz, 1H), 7.30-7.24 (m, 4H), 7.21-7.15 (m, 1H), 6.83 (d, J = 2.7 Hz, 1H), 3.61 (s, |
| | | | | | | 2H). |
| 84 | 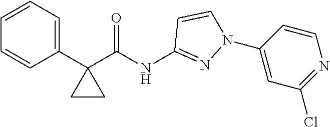 [SMILES: O=C(Nc1ccn(-c2ccnc(Cl)c2)n1)C1(c2ccccc2)CC1] | L | 23 | 0.061 | 339.12 | 1H NMR (400 MHz, CDCl₃) δ 8.65 (s, 1H), 7.77 (d, J = 2.0 Hz, 1H), 7.53 (d, J = 5.5 Hz, 1H), 6.98 (s, 1H), 6.88 (d, J = 5.6 Hz, 1H), 6.52 (ddd, J = 25.8, 18.3, 7.7 Hz, 5H), 6.02 (d, J = 2.0 Hz, 1H), 0.67-0.56 (m, 2H), 0.28 (d, J = 4.2 Hz, 2H). |
| 85 | 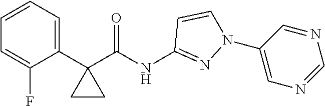 [SMILES: O=C(Nc1ccn(-c2cncnc2)n1)C1(c2ccccc2F)CC1] | G | 27 | 0.095 | 324.58 | 1H NMR (400 MHz, DMSO-d₆) δ 9.61 (s, 1H), 9.20 (d, J = 1.4 Hz, 2H), 9.07 (d, J = 1.4 Hz, 1H), 8.55 (t, J = 1.9 Hz, 1H), 7.52-7.44 (m, 1H), 7.44- 7.35 (m, 1H), 7.27-7.17 (m, 2H), 6.86 (t, J = 2.0 Hz, |
| | | | | | | 1H), 1.62 (dq, J = 6.5, 4.1, |
| | | | | | | 2.8 Hz, 2H), 1.20-1.10 (m, |
| | | | | | | 2H). |
| 86 | 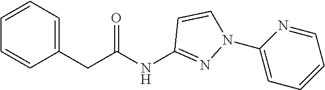 [SMILES: O=C(Cc1ccccc1)Nc1ccn(-c2ccccn2)n1] | B | 10 | 0.68 | 278.88 | 1H NMR (400 MHz, DMSO-d₆) δ 11.05 (s, 1H), 8.51 (d, J = 2.7 Hz, 1H), 8.44 (ddd, J = 4.9, 1.8, 0.8 Hz, 1H), 8.08-7.91 (m, 1H), 7.75 (dt, J = 8.3, 1.0 Hz, 1H), 7.38-7.32 (m, |
| | | | | | | 4H), 7.31 (ddd, J = 4.8, 3.2, |
| | | | | | | 1.0 Hz, 1H), 7.28-7.21 (m, |
| | | | | | | 1H), 6.82 (d, J = 2.7 Hz, |
| | | | | | | 1H), 3.68 (s, 2H). |
| 87 | 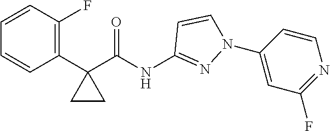 [SMILES: O=C(Nc1ccn(-c2ccnc(F)c2)n1)C1(c2ccccc2F)CC1] | N | 87 | 0.013 | 340.94 | 1H NMR (400 MHz, DMSO-d₆) δ 9.59 (s, 1H), 8.63 (d, J = 2.8 Hz, 1H), 8.25 (d, J = 5.7 Hz, 1H), 7.71 (dt, J = 5.7, 1.5 Hz, 1H), 7.55-7.44 (m, 2H), 7.44-7.33 (m, 1H), 7.28- 7.13 (m, 2H), 6.88 (d, J = 2.8 Hz, 1H), 1.71-1.54 (m, |
| | | | | | | 2H), 1.25-1.08 (m, 2H). |
| 88 | 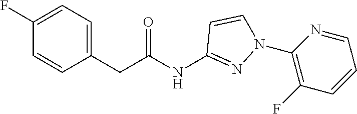 [SMILES: O=C(Cc1ccc(F)cc1)Nc1ccn(-c2ncccc2F)n1] | C | 32 | >5 | 315.53 | 1H NMR (400 MHz, DMSO-d₆) δ 11.09 (s, 1H), 8.43-8.22 (m, 2H), 7.98 (ddd, J = 11.7, 8.3, 1.4 Hz, 1H), 7.47 (ddd, J = 8.1, 4.7, 3.3 Hz, 1H), 7.41-7.32 (m, 2H), 7.21-7.09 (m, 2H), 6.84 (d, J = 2.7 Hz, 1H), |
| | | | | | | 3.67 (s, 2H). |
| 89 | 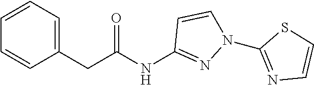 [SMILES: O=C(Cc1ccccc1)Nc1ccn(-c2nccs2)n1] | C | 17 | 0.34 | 285.52 | 1H NMR (400 MHz, DMSO-d₆) δ 11.19 (s, 1H), 8.38 (d, J = 2.7 Hz, 1H), 7.60 (d, J = 3.5 Hz, 1H), 7.49 (d, J = 3.5 Hz, 1H), 7.37-7.30 (m, 4H), 6.85 |
| | | | | | | (d, J = 2.7 Hz, 1H), 3.66 (s, |
| | | | | | | 2H). |
| 90 | 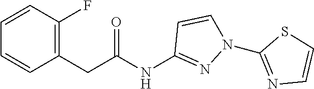 [SMILES: O=C(Cc1ccccc1F)Nc1ccn(-c2nccs2)n1] | C | 20 | 0.35 | 303.47 | 1H NMR (400 MHz, DMSO-d₆) δ 11.23 (s, 1H), 8.39 (d, J = 2.7 Hz, 1H), 7.61 (d, J = 3.4 Hz, 1H), 7.50 (d, J = 3.5 Hz, 1H), 7.43-7.27 (m, 2H), 7.22- |
| | | | | | | 7.12 (m, 2H), 6.85 (d, J = |
| | | | | | | 2.6 Hz, 1H), 3.76 (s, 2H). |
| 91 | 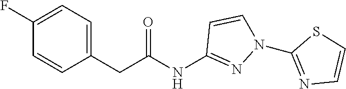 [SMILES: O=C(Cc1ccc(F)cc1)Nc1ccn(-c2nccs2)n1] | C | 28 | 1.9 | 303.47 | 1H NMR (400 MHz, DMSO-d₆) δ 11.20 (s, 1H), 8.38 (d, J = 2.7 Hz, 1H), 7.60 (d, J = 3.5 Hz, 1H), 7.50 (d, J = 3.4 Hz, 1H), 7.41-7.32 (m, 2H), 7.20- 7.09 (m, 2H), 6.85 (d, J = |
| | | | | | | 2.7 Hz, 1H), 3.66 (s, 2H). |
| 92 | 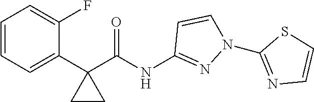 [SMILES: O=C(Nc1ccn(-c2nccs2)n1)C1(c2ccccc2F)CC1] | C | 4 | 0.038 | 329.53 | 1H NMR (400 MHz, DMSO-d₆) δ 9.83 (s, 1H), 8.37 (d, J = 2.7 Hz, 1H), 7.59 (d, J = 3.4 Hz, 1H), 7.50-7.43 (m, 2H), 7.39 (tdd, J = 7.5, 5.3, 1.8 Hz, 1H), 7.25-7.15 (m, 2H), 6.83 (d, J = 2.8 Hz, 1H), |
| | | | | | | 1.62 (q, J = 4.3 Hz, 2H), |
| | | | | | | 1.16 (q, J = 4.4 Hz, 2H). |
| 93 | 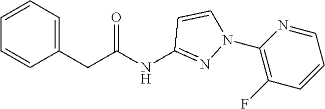 [SMILES: O=C(Cc1ccccc1)Nc1ccn(-c2ncccc2F)n1] | C | 24 | >10 | 297.53 | 1H NMR (400 MHz, DMSO-d₆) δ 11.08 (s, 1H), 8.35 (dd, J = 5.2, 2.0 Hz, 2H), 7.98 (ddd, J = 11.7, 8.3, 1.4 Hz, 1H), 7.47 (ddd, J = 8.1, 4.6, 3.3 Hz, 1H), 7.40-7.28 (m, 4H), 7.28- 7.21 (m, 1H), 6.85 (d, J = |
| | | | | | | 2.7 Hz, 1H), 3.67 (s, 2H). |
| 94 | 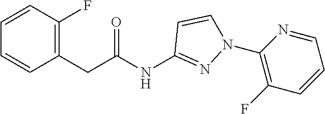 [SMILES: O=C(Cc1ccccc1F)Nc1ccn(-c2ncccc2F)n1] | C | 31 | >10 | 315.53 | 1H NMR (400 MHz, DMSO-d₆) δ 11.12 (s, 1H), 8.35 (dd, J = 5.4, 3.6 Hz, 2H), 7.98 (ddd, J = 11.6, 8.2, 1.4 Hz, 1H), 7.47 (ddd, J = 8.1, 4.7, 3.3 Hz, 1H), 7.43-7.23 (m, 2H), 7.22- 7.08 (m, 2H), 6.84 (d, J = |
| | | | | | | 2.7 Hz, 1H), 3.77 (s, 2H). |
| 95 | 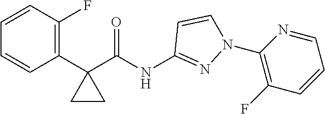 [SMILES: O=C(Nc1ccn(-c2ncccc2F)n1)C1(c2ccccc2F)CC1] | C | 13 | 0.39 | 341.54 | 1H NMR (400 MHz, DMSO-d₆) δ 9.51 (s, 1H), 8.32 (ddd, J = 6.3, 2.9, 1.8 Hz, 2H), 7.96 (ddd, J = 11.6, 8.3, 1.4 Hz, 1H), 7.51- 7.44 (m, 2H), 7.38 (ddd, J = 7.5, 5.3, 1.9 Hz, 1H), 7.26-7.14 (m, 2H), 6.81 |
| | | | | | | (d, J = 2.7 Hz, 1H), 1.61 (q, |
| | | | | | | J = 4.3 Hz, 2H), 1.16 (q, J = |
| | | | | | | 4.3 Hz, 2H). |
| 96 | 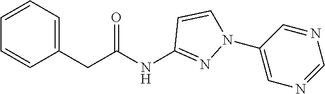 [SMILES: O=C(Cc1ccccc1)Nc1ccn(-c2cncnc2)n1] | C | 15 | 2.4 | 280.57 | 1H NMR (400 MHz, DMSO-d₆) δ 11.12 (s, 1H), 9.23 (s, 2H), 9.10 (s, 1H), 8.57 (d, J = 2.6 Hz, 1H), 7.33 (d, J = 5.7 Hz, 4H), 7.25 (ddd, J = 8.7, 5.6, 2.8 Hz, 1H), 6.88 (d, J = 2.7 Hz, |
| | | | | | | 1H), 3.67 (s, 2H). |
| 97 | 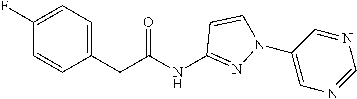 [SMILES: O=C(Cc1ccc(F)cc1)Nc1ccn(-c2cncnc2)n1] | C | 23 | >10 | 298.52 | 1H NMR (400 MHz, DMSO-d₆) δ 11.06 (s, 1H), 9.16 (s, 2H), 9.03 (s, 1H), 8.50 (d, J = 2.7 Hz, 1H), 7.30 (dd, J = 8.5, 5.7 Hz, 2H), 7.09 (t, J = 8.9 Hz, 2H), 6.81 (d, J = 2.7 Hz, 1H), 3.60 (s, 2H). |
| 98 | 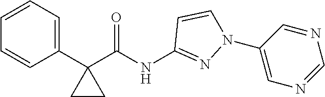 [SMILES: O=C(Nc1ccn(-c2cncnc2)n1)C1(c2ccccc2)CC1] | C | 26 | 0.11 | 306.53 | 1H NMR (400 MHz, DMSO-d₆) δ 9.44 (s, 1H), 9.12 (s, 2H), 9.00 (s, 1H), 8.48 (d, J = 2.6 Hz, 1H), 7.41-7.22 (m, 5H), 6.81 (d, J = 2.7 Hz, 1H), 1.41 (q, J = 4.2 Hz, 2H), 1.07 (q, J = 4.3 Hz, 2H). |
| 99 | 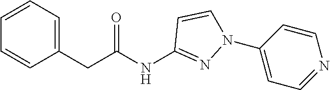 [SMILES: O=C(Cc1ccccc1)Nc1ccn(-c2ccncc2)n1] | C | 34 | 0.76 | 279.58 | 1H NMR (400 MHz, DMSO-d₆) δ 11.10 (s, 1H), 8.62 (dd, J = 4.8, 1.5 Hz, 3H), 7.76 (dd, J = 4.8, 1.5 Hz, 2H), 7.39-7.21 (m, 5H), 6.87 (d, J = 2.6 Hz, 1H), 3.68 (s, 2H). |
| 100 | 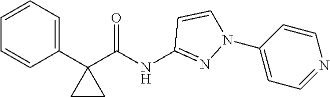 [SMILES: O=C(Nc1ccn(-c2ccncc2)n1)C1(c2ccccc2)CC1] | C | 30 | 0.033 | 305.59 | 1H NMR (400 MHz, DMSO-d₆) δ 9.47 (s, 1H), 8.65-8.53 (m, 3H), 7.79- 7.70 (m, 2H), 7.56-7.28 (m, 5H), 6.87 (d, J = 2.7 Hz, 1H), 1.54-1.42 (m, 2H), 1.20-1.08 (m, 2H). |
| 101 | 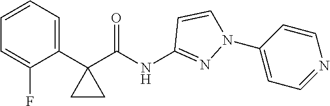 [SMILES: O=C(Nc1ccn(-c2ccncc2)n1)C1(c2ccccc2F)CC1] | C | 21 | 0.075 | 323.54 | 1H NMR (400 MHz, DMSO-d₆) δ 9.58 (s, 1H), 8.65-8.53 (m, 3H), 8.26- 7.93 (m, 1H), 7.74 (dd, J = 4.8, 1.5 Hz, 2H), 7.55- 7.45 (m, 1H), 7.45-7.36 (m, 1H), 7.27-7.16 (m, 2H), 6.84 (d, J = 2.7 Hz, |
| | | | | | | 1H), 3.62 (dt, J = 13.2, 6.6 |
| | | | | | | Hz, 1H), 3.14 (q, J = 7.4 Hz, |
| | | | | | | 1H), 1.62 (q, J = 4.3 Hz, |
| | | | | | | 2H), 1.22-1.14 (m, 2H). |
| 102 | 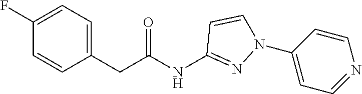 [SMILES: O=C(Cc1ccc(F)cc1)Nc1ccn(-c2ccncc2)n1] | C | 27 | 3.6 | 297.15 | 1H NMR (400 MHz, DMSO-d₆) δ 11.10 (s, 1H), 8.62 (dd, J = 4.8, 1.7 Hz, 2H), 7.76 (dd, J = 4.8, 1.5 Hz, 2H), 7.48-7.02 (m, 5H), 6.87 (d, J = 2.6 Hz, 1H), 3.67 (s, 2H). |
| 103 | 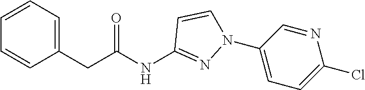 [SMILES: O=C(Cc1ccccc1)Nc1ccn(-c2ccc(Cl)nc2)n1] | C | 10 | 0.58 | 313.21 | 1H NMR (400 MHz, DMSO-d₆) δ 11.07 (s, 1H), 8.86 (d, J = 2.9 Hz, 1H), 8.51 (d, J = 2.6 Hz, 1H), 8.22 (dd, J = 8.7, 2.9 Hz, 1H), 7.67 (d, J = 8.7 Hz, 1H), 7.38-7.29 (m, 4H), |
| | | | | | | 7.29-7.21 (m, 1H), 6.84 |
| | | | | | | (d, J = 2.6 Hz, 1H), 3.67 (s, |
| | | | | | | 2H). |
| 104 | 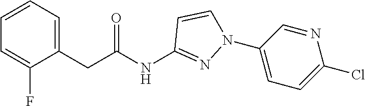 [SMILES: O=C(Cc1ccccc1F)Nc1ccn(-c2ccc(Cl)nc2)n1] | C | 8 | 0.32 | 331.21 | 1H NMR (400 MHz, DMSO-d₆) δ 11.11 (s, 1H), 8.87 (d, J = 2.9 Hz, 1H), 8.52 (d, J = 2.7 Hz, 1H), 8.23 (dd, J = 8.7, 2.9 Hz, 1H), 7.67 (d, J = 8.7 Hz, 1H), 7.47-7.23 (m, 2H), 7.23-7.06 (m, 2H), 6.83 |
| | | | | | | (d, J = 2.7 Hz, 1H), 3.77 (s, |
| | | | | | | 2H). |
| 105 | 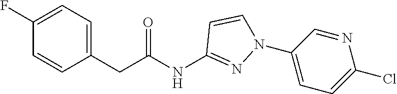 [SMILES: O=C(Cc1ccc(F)cc1)Nc1ccn(-c2ccc(Cl)nc2)n1] | C | 6 | >5 | 331.21 | 1H NMR (400 MHz, DMSO-d₆) δ 11.07 (s, 1H), 8.86 (d, J = 2.8 Hz, 1H), 8.51 (d, J = 2.7 Hz, 1H), 8.22 (dd, J = 8.7, 2.9 Hz, 1H), 7.67 (d, J = 8.7 Hz, 1H), 7.46-7.30 (m, 2H), |
| | | | | | | 7.22-7.07 (m, 2H), 6.83 |
| | | | | | | (d, J = 2.6 Hz, 1H), 3.66 (s, |
| | | | | | | 2H). |
| 106 | 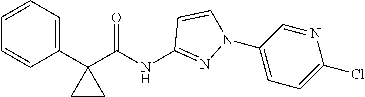 [SMILES: O=C(Nc1ccn(-c2ccc(Cl)nc2)n1)C1(c2ccccc2)CC1] | C | 28 | 0.25 | 339.20 | 1H NMR (400 MHz, DMSO-d₆) δ 9.42 (s, 1H), 8.83 (d, J = 2.8 Hz, 1H), 8.50 (d, J = 2.7 Hz, 1H), 8.19 (dd, J = 8.7, 2.9 Hz, 1H), 7.62 (d, J = 8.7 Hz, 1H), 7.45 (d, J = 7.1 Hz, |
| | | | | | | 2H), 7.39 (t, J = 7.5 Hz, |
| | | | | | | 2H), 7.33 (dd, J = 8.3, 5.9 |
| | | | | | | Hz, 1H), 6.84 (d, J = 2.7 Hz, |
| | | | | | | 1H), 1.48 (q, J = 4.2 Hz, |
| | | | | | | 2H), 1.14 (q, J = 4.3 Hz, |
| | | | | | | 2H). |
| 107 | 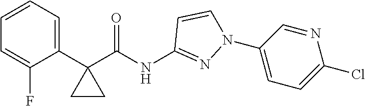 [SMILES: O=C(Nc1ccn(-c2ccc(Cl)nc2)n1)C1(c2ccccc2F)CC1] | C | 14 | 0.084 | 356.98 | 1H NMR (400 MHz, DMSO-d₆) δ 9.53 (s, 1H), 8.83 (d, J = 2.8 Hz, 1H), 8.49 (d, J = 2.6 Hz, 1H), 8.19 (dd, J = 8.7, 2.9 Hz, 1H), 7.62 (d, J = 8.7 Hz, 1H), 7.53-7.44 (m, 1H), 7.40 (q, J = 6.9, 6.1 Hz, |
| | | | | | | 1H), 7.27-7.16 (m, 2H), |
| | | | | | | 6.81 (d, J = 2.6 Hz, 1H), |
| | | | | | | 1.60 (q, J = 4.3 Hz, 2H), |
| | | | | | | 1.16 (q, J = 4.3 Hz, 2H). |
| 108 | 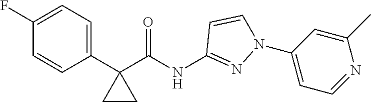 [SMILES: Cc1cc(-n2ccc(NC(=O)C3(c4ccc(F)cc4)CC3)n2)ccn1] | C | 9 | 0.77 | 337.63 | 1H NMR (400 MHz, DMSO-d₆) δ 9.58 (s, 1H), 8.57 (d, J = 2.7 Hz, 1H), 8.45 (d, J = 5.6 Hz, 1H), 7.65 (d, J = 2.1 Hz, 1H), 7.56 (dd, J = 5.7, 2.1 Hz, 1H), 7.53-7.41 (m, 2H), |
| | | | | | | 7.28-7.14 (m, 2H), 6.84 |
| | | | | | | (d, J = 2.6 Hz, 1H), 1.49 (q, |
| | | | | | | J = 4.2 Hz, 2H), 1.12 (q, J = |
| | | | | | | 4.3 Hz, 2H). |
| 109 | 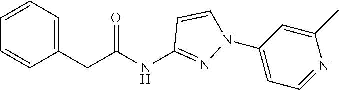 [SMILES: Cc1cc(-n2ccc(NC(=O)Cc3ccccc3)n2)ccn1] | C | 20 | >10 | 293.62 | 1H NMR (400 MHz, DMSO-d₆) δ 11.07 (s, 1H), 8.57 (d, J = 2.7 Hz, 1H), 8.47 (d, J = 5.6 Hz, 1H), 7.65 (d, J = 2.1 Hz, 1H), 7.56 (dd, J = 5.7, 2.2 Hz, 1H), 7.38-7.29 (m, 4H), 7.25 (td, J = 5.9, 2.7 Hz, 1H), 6.85 (d, J = 2.7 Hz, 1H), 3.67 (s, 2H), 2.51 (s, 3H). |
| 110 | 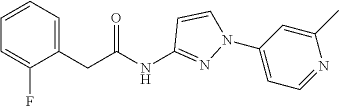 [SMILES: Cc1cc(-n2ccc(NC(=O)Cc3ccccc3F)n2)ccn1] | C | 12 | >10 | 311.57 | 1H NMR (400 MHz, DMSO-d₆) δ 11.11 (s, 1H), 8.58 (d, J = 2.7 Hz, 1H), 8.48 (d, J = 5.6 Hz, 1H), 7.65 (d, J = 2.1 Hz, 1H), 7.57 (dd, J = 5.7, 2.1 Hz, 1H), 7.39 (td, J = 7.7, 1.7 |
| | | | | | | Hz, 1H), 7.36-7.27 (m, |
| | | | | | | 1H), 7.22-7.12 (m, 2H), |
| | | | | | | 6.84 (d, J = 2.7 Hz, 1H), |
| | | | | | | 3.77 (s, 2H), 2.52 (s, 3H). |
| 111 | 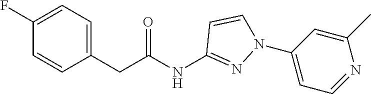 [SMILES: Cc1cc(-n2ccc(NC(=O)Cc3ccc(F)cc3)n2)ccn1] | C | 21 | >10 | 311.57 | 1H NMR (400 MHz, DMSO-d₆) δ 11.08 (s, 1H), 8.57 (d, J = 2.8 Hz, 1H), 8.47 (d, J = 5.6 Hz, 1H), 7.65 (d, J = 2.1 Hz, 1H), 7.56 (dd, J = 5.6, 2.1 Hz, 1H), 7.37 (dd, J = 8.4, 5.7 |
| | | | | | | Hz, 2H), 7.25-7.08 (m, |
| | | | | | | 2H), 6.84 (d, J = 2.7 Hz, |
| | | | | | | 1H), 3.67 (s, 2H), 2.51 (s, |
| | | | | | | 3H). |
| 112 | 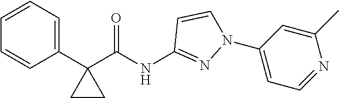 [SMILES: Cc1cc(-n2ccc(NC(=O)C3(c4ccccc4)CC3)n2)ccn1] | C | 19 | 0.91 | 319.58 | 1H NMR (400 MHz, DMSO-d₆) δ 9.36 (s, 1H), 8.56 (d, J = 2.8 Hz, 1H), 8.43 (d, J = 5.6 Hz, 1H), 7.61 (d, J = 2.1 Hz, 1H), 7.53 (dd, J = 5.7, 2.1 Hz, 1H), 7.49-7.43 (m, 2H), |
| | | | | | | 7.40 (t, J = 7.4 Hz, 2H), |
| | | | | | | 7.35 (d, J = 7.1 Hz, 1H), |
| | | | | | | 6.85 (d, J = 2.7 Hz, 1H), |
| | | | | | | 2.55 (s, 3H), 1.49 (q, J = |
| | | | | | | 4.2 Hz, 2H), 1.14 (q, J = 4.2 |
| | | | | | | Hz, 2H). |
| 113 | 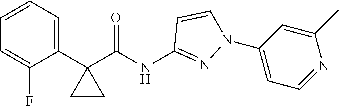 [SMILES: Cc1cc(-n2ccc(NC(=O)C3(c4ccccc4F)CC3)n2)ccn1] | C | 10 | 1.6 | 337.58 | 1H NMR (400 MHz, DMSO-d₆) δ 9.50 (s, 1H), 8.55 (d, J = 2.7 Hz, 1H), 8.43 (d, J = 5.6 Hz, 1H), 7.63 (d, J = 2.1 Hz, 1H), 7.54 (dd, J = 5.7, 2.1 Hz, 1H), 7.48 (td, J = 7.8, 1.9 |
| | | | | | | Hz, 1H), 7.41 (tdd, J = 7.3, |
| | | | | | | 5.2, 1.7 Hz, 1H), 7.27- |
| | | | | | | 7.16 (m, 2H), 6.82 (d, J = |
| | | | | | | 2.7 Hz, 1H), 2.48 (s, 3H), |
| | | | | | | 1.61 (q, J = 4.3 Hz, 2H), |
| | | | | | | 1.17 (q, J = 4.3 Hz, 2H). |
| 114 | 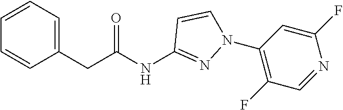 [SMILES: O=C(Cc1ccccc1)Nc1ccn(-c2cc(F)ncc2F)n1] | L | 17 | 1.2 | 315.09 | 1H NMR (400 MHz, CDCl₃) δ 10.29 (s, 1H), 7.51 (s, 1H), 7.42 (s, 1H), 7.36 (s, 1H), 6.63 (d, J = 5.3 Hz, 1H), 6.48 (dt, J = 14.8, 7.4 Hz, 3H), 6.40 (d, J = 6.6 Hz, 1H), 6.14 (s, 1H), 2.85 (s, 2H). |
| 115 | 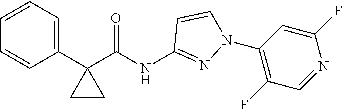 [SMILES: O=C(Nc1ccn(-c2cc(F)ncc2F)n1)C1(c2ccccc2)CC1] | L | 7 | 0.12 | 341.10 | 1H NMR (400 MHz, DMSO-d₆) δ 9.61 (s, 1H), 8.42 (dd, J = 3.6, 1.0 Hz, 1H), 8.32 (t, J = 2.5 Hz, 1H), 7.49 (dd, J = 5.2, 1.6 Hz, 1H), 7.47-7.43 (m, 2H), 7.39 (t, J = 7.4 Hz, 2H), 7.36-7.30 (m, 1H), |
| | | | | | | 6.95 (d, J = 2.8 Hz, 1H), |
| | | | | | | 1.49 (q, J = 4.2 Hz, 2H), |
| | | | | | | 1.15 (q, J = 4.3 Hz, 2H). |
| 116 | 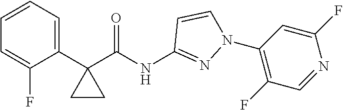 [SMILES: O=C(Nc1ccn(-c2cc(F)ncc2F)n1)C1(c2ccccc2F)CC1] | L | 3 | 0.076 | 358.96 | 1H NMR (400 MHz, DMSO-d₆) δ 9.71 (s, 1H), 8.43 (d, J = 2.9 Hz, 1H), 8.31 (s, 1H), 7.55-7.45 (m, 2H), 7.43-7.36 (m, 1H), 7.21 (dd, J = 12.7, 6.3 Hz, 2H), 6.93 (d, J = 2.7 Hz, 1H), 1.64-1.59 (m, 2H), |
| | | | | | | 1.18 (q, J = 4.2 Hz, 2H). |
| 117 | 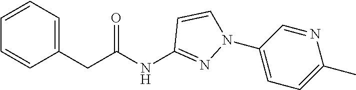 [SMILES: Cc1ccc(-n2ccc(NC(=O)Cc3ccccc3)n2)cn1] | G | 62 | >5 | 293.13 | 1H NMR (400 MHz, DMSO-d₆) δ 11.02 (s, 1H), 8.88 (d, J = 2.7 Hz, 1H), 8.42 (d, J = 2.6 Hz, 1H), 8.03 (dd, J = 8.4, 2.8 Hz, 1H), 7.38 (d, J = 8.5 Hz, 1H), 7.36-7.29 (m, 4H), |
| | | | | | | 7.29-7.21 (m, 1H), 6.79 |
| | | | | | | (d, J = 2.6 Hz, 1H), 3.66 (s, |
| | | | | | | 2H), 2.50 (s, 3H). |
| 118 | 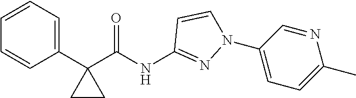 [SMILES: Cc1ccc(-n2ccc(NC(=O)C3(c4ccccc4)CC3)n2)cn1] | G | 58 | 1.3 | 319.09 | 1H NMR (400 MHz, DMSO-d₆) δ 9.33 (s, 1H), 8.84 (d, J = 2.7 Hz, 1H), 8.42 (d, J = 2.6 Hz, 1H), 7.99 (dd, J = 8.4, 2.8 Hz, 1H), 7.45 (d, J = 7.2 Hz, 2H), 7.40 (t, J = 7.4 Hz, |
| | | | | | | 2H), 7.34 (d, J = 8.1 Hz, |
| | | | | | | 2H), 6.79 (d, J = 2.5 Hz, |
| | | | | | | 1H), 2.47 (s, 3H), 1.48 (q, |
| | | | | | | J = 4.2 Hz, 2H), 1.13 (q, J = |
| | | | | | | 4.3 Hz, 2H). |
| 119 | 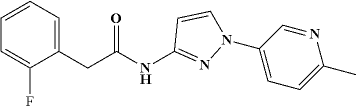 [SMILES: Cc1ccc(-n2ccc(NC(=O)Cc3ccccc3F)n2)cn1] | G | 52 | 3.5 | 311.04 | 1H NMR (400 MHz, DMSO-d₆) δ 11.06 (s, 1H), 8.89 (d, J = 2.7 Hz, 1H), 8.43 (d, J = 2.6 Hz, 1H), 8.04 (dd, J = 8.5, 2.8 Hz, 1H), 7.38 (dd, J = 8.0, 4.6 Hz, 2H), 7.31 (dd, J = 8.0, |
| | | | | | | 5.8 Hz, 1H), 7.23-7.12 |
| | | | | | | (m, 2H), 6.78 (d, J = 2.5 |
| | | | | | | Hz, 1H), 3.76 (s, 2H), 2.50 |
| | | | | | | (s, 3H). |
| 120 | 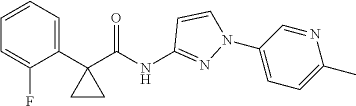 [SMILES: Cc1ccc(-n2ccc(NC(=O)C3(c4ccccc4F)CC3)n2)cn1] | G | 31 | 0.62 | 337.04 | 1H NMR (400 MHz, DMSO-d₆) δ 9.43 (s, 1H), 8.84 (d, J = 2.7 Hz, 1H), 8.40 (d, J = 2.6 Hz, 1H), 7.99 (dd, J = 8.4, 2.7 Hz, 1H), 7.55-7.43 (m, 1H), 7.38 (dd, J = 7.9, 5.8 Hz, |
| | | | | | | 1H), 7.33 (d, J = 8.5 Hz, |
| | | | | | | 1H), 7.20 (dd, J = 11.2, 7.9 |
| | | | | | | Hz, 2H), 6.75 (d, J = 2.5 Hz, |
| | | | | | | 1H), 2.47 (s, 3H), 1.59 (q, J = |
| | | | | | | 4.3 Hz, 2H), 1.14 (q, J = |
| | | | | | | 4.4 Hz, 2H). |
| 121 | 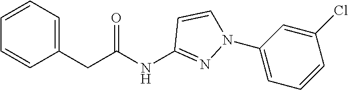 [SMILES: O=C(Cc1ccccc1)Nc1ccn(-c2cccc(Cl)c2)n1] | F | 60 | 0.65 | 312.13 | 1H NMR (400 MHz, DMSO-d₆) δ 11.00 (s, 1H), 8.48 (d, J = 2.6 Hz, 1H), 7.87 (t, J = 2.1 Hz, 1H), 7.75 (dd, J = 8.2, 2.1 Hz, 1H), 7.51 (t, J = 8.1 Hz, 1H), 7.33 (dd, J = 7.3, 3.7 |
| | | | | | | Hz, 5H), 7.25 (td, J = 5.8, |
| | | | | | | 2.6 Hz, 1H), 6.79 (d, J = 2.5 |
| | | | | | | Hz, 1H), 3.66 (s, 2H). |
| 122 | 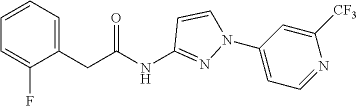 [SMILES: O=C(Cc1ccccc1F)Nc1ccn(-c2ccnc(C(F)(F)F)c2)n1] | C | 33 | 1.6 | 364.99 | 1H NMR (400 MHz, DMSO-d₆) δ 11.21 (s, 1H), 8.79 (d, J = 5.6 Hz, 1H), 8.76 (d, J = 2.8 Hz, 1H), 8.22 (d, J = 2.1 Hz, 1H), 8.04 (dd, J = 5.6, 2.1 Hz, 1H), 7.38 (dd, J = 8.6, 6.8 |
| | | | | | | Hz, 1H), 7.31 (dd, J = 7.9, |
| | | | | | | 5.8 Hz, 1H), 7.24-7.05 |
| | | | | | | (m, 2H), 6.91 (d, J = 2.7 |
| | | | | | | Hz, 1H), 3.77 (s, 2H). |
| 123 | 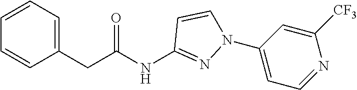 [SMILES: O=C(Cc1ccccc1)Nc1ccn(-c2ccnc(C(F)(F)F)c2)n1] | C | 28 | 1.5 | 347.04 | 1H NMR (400 MHz, DMSO-d₆) δ 11.17 (s, 1H), 8.78 (d, J = 5.6 Hz, 1H), 8.75 (d, J = 2.8 Hz, 1H), 8.21 (d, J = 2.0 Hz, 1H), 8.03 (dd, J = 5.6, 2.1 Hz, |
| | | | | | | 1H), 7.43-7.13 (m, 5H), |
| | | | | | | 6.92 (d, J = 2.7 Hz, 1H), |
| | | | | | | 3.67 (s, 2H). |
| 124 | 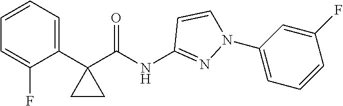 [SMILES: O=C(Nc1ccn(-c2cccc(F)c2)n1)C1(c2ccccc2F)CC1] | B | 2 | 0.041 | 339.89 | 1H NMR (400 MHz, DMSO-d₆) δ 9.43 (s, 1H), 8.44 (d, J = 2.6 Hz, 1H), 7.60 (dd, J = 10.2, 2.6 Hz, 2H), 7.54-7.44 (m, 2H), 7.40 (tdd, J = 7.5, 5.3, 1.7 Hz, 1H), 7.27-7.14 (m, 2H), 7.07 (td, J = 7.7, 7.0, |
| | | | | | | 2.0 Hz, 1H), 6.75 (d, J = 2.6 |
| | | | | | | Hz, 1H), 1.60 (q, J = 4.3 Hz, |
| | | | | | | 2H), 1.15 (q, J = 4.3 Hz, |
| | | | | | | 2H). |
| 125 | 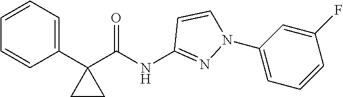 [SMILES: O=C(Nc1ccn(-c2cccc(F)c2)n1)C1(c2ccccc2)CC1] | B | 28 | 0.071 | 321.92 | 1H NMR (400 MHz, DMSO-d₆) δ 9.32 (s, 1H), 8.46 (d, J = 2.7 Hz, 1H), 7.65-7.54 (m, 2H), 7.49- 7.31 (m, 6H), 7.08 (td, J = 8.6, 2.2 Hz, 1H), 6.79 (d, J = 2.6 Hz, 1H), 1.48 (q, J = |
| | | | | | | 4.2 Hz, 2H), 1.13 (q, J = 4.3 |
| | | | | | | Hz, 2H). |
| 126 | 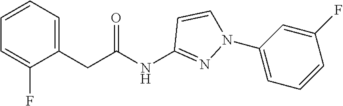 [SMILES: O=C(Cc1ccccc1F)Nc1ccn(-c2cccc(F)c2)n1] | B | 35 | 0.50 | 313.86 | 1H NMR (400 MHz, DMSO-d₆) δ 11.04 (s, 1H), 8.47 (d, J = 2.6 Hz, 1H), 7.72-7.60 (m, 2H), 7.52 (q, J = 7.8 Hz, 1H), 7.38 (td, J = 7.6, 1.7 Hz, 1H), 7.35-7.25 (m, 1H), 7.25- |
| | | | | | | 7.00 (m, 3H), 6.78 (d, J = |
| | | | | | | 2.6 Hz, 1H), 3.76 (s, 2H). |
| 127 | 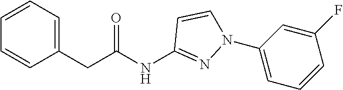 [SMILES: O=C(Cc1ccccc1)Nc1ccn(-c2cccc(F)c2)n1] | B | 14 | 0.81 | 295.9 | 1H NMR (400 MHz, DMSO-d₆) δ 10.36 (s, 1H), 8.72 (d, J = 2.6 Hz, 1H), 8.05 (dd, J = 8.2, 1.3 Hz, 1H), 8.01 7.91 (m, 2H), 7.87 (d, J = 7.3 Hz, 2H), |
| | | | | | | 7.79 (dd, J = 10.1, 4.7 Hz, |
| | | | | | | 2H), 7.70 (t, J = 7.3 Hz, |
| | | | | | | 1H), 7.47 (td, J = 8.3, 2.1 |
| | | | | | | Hz, 1H), 7.38 (d, J = 2.6 Hz, |
| | | | | | | 1H), 4.25 (s, 2H). |
| 128 | 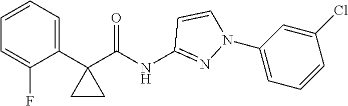 [SMILES: O=C(Nc1ccn(-c2cccc(Cl)c2)n1)C1(c2ccccc2F)CC1] | F | 4 | 0.23 | 355.95 | 1H NMR (400 MHz, DMSO-d₆) δ 9.42 (s, 1H), 8.46 (d, J = 2.6 Hz, 1H), 7.84 (t, J = 2.1 Hz, 1H), 7.72 (dd, J = 8.3, 2.1 Hz, 1H), 7.48 (td, J = 8.2, 2.3 Hz, 2H), 7.40 (dd, J = 7.9, |
| | | | | | | 6.0 Hz, 1H), 7.30 (dd, J = |
| | | | | | | 8.1, 1.9 Hz, 1H), 7.21 (dd, |
| | | | | | | J = 11.3, 7.9 Hz, 2H), 6.76 |
| | | | | | | (d, J = 2.6 Hz, 1H), 1.60 (q, |
| | | | | | | J = 4.3 Hz, 2H), 1.16 (q, J = |
| | | | | | | 4.3 Hz, 2H). |
| 129 | 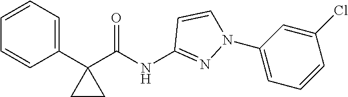 [SMILES: O=C(Nc1ccn(-c2cccc(Cl)c2)n1)C1(c2ccccc2)CC1] | F | 20 | 0.70 | 338.16 | 1H NMR (400 MHz, DMSO-d₆) δ 9.32 (s, 1H), 8.47 (d, J = 2.6 Hz, 1H), 7.83 (t, J = 2.1 Hz, 1H), 7.71 (dd, J = 8.1, 2.0 Hz, 1H), 7.47 (dd, J = 9.2, 7.6 Hz, 3H), 7.40 (t, J = 7.4 Hz, |
| | | | | | | 2H), 7.36-7.27 (m, 2H), |
| | | | | | | 6.79 (d, J = 2.6 Hz, 1H), |
| | | | | | | 1.48 (q, J = 4.3 Hz, 2H), |
| | | | | | | 1.13 (q, J = 4.3 Hz, 2H). |
| 130 | 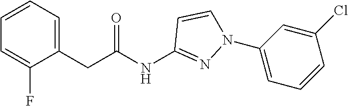 [SMILES: O=C(Cc1ccccc1F)Nc1ccn(-c2cccc(Cl)c2)n1] | F | 39 | 1.5 | 330.13 | 1H NMR (400 MHz, DMSO-d₆) δ 11.04 (s, 1H), 8.49 (d, J = 2.6 Hz, 1H), 7.88 (t, J = 2.0 Hz, 1H), 7.76 (dd, J = 8.3, 2.1 Hz, 1H), 7.51 (t, J = 8.1 Hz, 1H), 7.44-7.23 (m, 3H), |
| | | | | | | 7.25-7.04 (m, 2H), 6.78 |
| | | | | | | (d, J = 2.6 Hz, 1H), 3.76 (s, |
| | | | | | | 2H). |
| 131 | 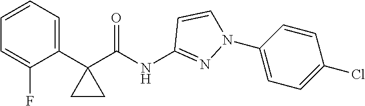 [SMILES: O=C(Nc1ccn(-c2ccc(Cl)cc2)n1)C1(c2ccccc2F)CC1] | F | 5 | 0.48 | 356.13 | 1H NMR (400 MHz, DMSO-d₆) δ 9.38 (s, 1H), 8.40 (d, J = 2.6 Hz, 1H), 7.82-7.73 (m, 2H), 7.46 (dd, J = 42.0, 7.8 Hz, 3H), 7.23 (d, J = 7.7 Hz, 2H), 6.74 (d, J = 2.6 Hz, 1H), |
| | | | | | | 1.60 (q, J = 4.3 Hz, 2H), |
| | | | | | | 1.16 (q, J = 4.4 Hz, 2H). |
| 132 | 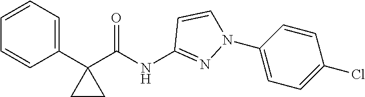 [SMILES: O=C(Nc1ccn(-c2ccc(Cl)cc2)n1)C1(c2ccccc2)CC1] | F | 33 | >5 | 338.12 | 1H NMR (400 MHz, DMSO-d₆) δ 9.23 (s, 1H), 8.39 (d, J = 2.6 Hz, 1H), 7.80-7.66 (m, 2H), 7.49 (d, J = 8.8 Hz, 2H), 7.39 (ddd, J = 26.2, 18.6, 7.2 Hz, 5H), 6.76 (d, J = 2.6 Hz, |
| | | | | | | 1H), 1.47 (q, J = 4.3 Hz, |
| | | | | | | 2H), 1.13 (p, J = 5.0, 4.5 |
| | | | | | | Hz, 2H). |
| 133 | 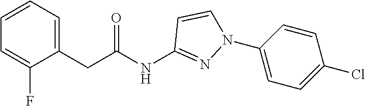 [SMILES: O=C(Cc1ccccc1F)Nc1ccn(-c2ccc(Cl)cc2)n1] | F | 82 | >5 | 330.09 | 1H NMR (400 MHz, DMSO-d₆) δ 11.02 (s, 1H), 8.41 (d, J = 2.6 Hz, 1H), 7.78 (d, J = 8.8 Hz, 2H), 7.54 (d, J = 8.8 Hz, 2H), 7.34 (dt, J = 28.6, 7.2 Hz, 2H), 7.21-7.06 (m, 2H), |
| | | | | | | 6.76 (d, J = 2.5 Hz, 1H), |
| | | | | | | 3.75 (s, 2H). |
| 134 | 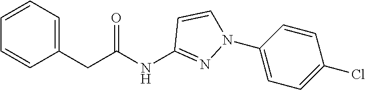 [SMILES: O=C(Cc1ccccc1)Nc1ccn(-c2ccc(Cl)cc2)n1] | F | 98 | >5 | 311.98 | 1H NMR (400 MHz, DMSO-d₆) δ 10.98 (s, 1H), 8.39 (d, J = 2.6 Hz, 1H), 7.77 (d, J = 8.7 Hz, 2H), 7.54 (d, J = 8.7 Hz, 2H), 7.38-7.16 (m, 5H), 6.76 |
| | | | | | | (d, J = 2.5 Hz, 1H), 3.65 (s, |
| | | | | | | 2H). |
| 135 | 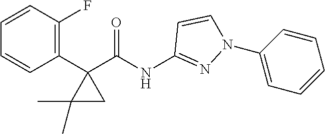 [SMILES: CC1(C)CC1(C(=O)Nc1ccn(-c2ccccc2)n1)c1ccccc1F] | D | 2 | 0.68 | 350.44 | 1H NMR (400 MHz, DMSO-d₆) δ 9.93 (s, 1H), 8.37 (d, J = 2.6 Hz, 1H), 7.75 (m, 3H), 7.50-7.39 (m, 2H), 7.35 (m, 1H), 7.28- 7.17 (m, 3H), 6.73 (d, J = 2.6 Hz, 1H), 1.67 (d, J = 5.3 Hz, 1H), 1.25 (s, 3H), 0.96 (m, 4H). |
| 136 | 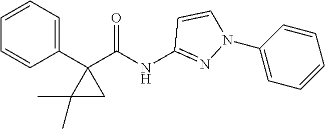 [SMILES: CC1(C)CC1(C(=O)Nc1ccn(-c2ccccc2)n1)c1ccccc1] | D | 37 | 0.94 | 332.43 | 1H NMR (400 MHz, DMSO-d₆) δ 10.42 (s, 1H), 8.36 (d, J = 2.4 Hz, 1H), 7.79-7.69 (m, 2H), 7.66- 7.56 (m, 2H), 7.52-7.41 (m, 2H), 7.34 (m, 2H), 7.30- 7.21 (m, 2H), 6.73 (d, J = 2.4 Hz, 1H), 1.41 (d, J = 5.1 Hz, 1H), 1.23 (s, 3H), 1.03 |
| | | | | | | (d, J = 5.1 Hz, 1H), 0.85 (s, |
| | | | | | | 3H). |
| 137 | 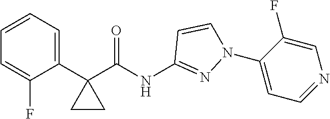 [SMILES: O=C(Nc1ccn(-c2ccncc2F)n1)C1(c2ccccc2F)CC1] | F | 8 | 0.12 | 341.15 | 1H NMR (400 MHz, DMSOd₆) δ 9.65 (s, 1H), 8.72 (d, J = 4.1 Hz, 1H), 8.45 (d, J = 5.4 Hz, 1H), 8.27 (d, J = 2.8 Hz, 1H), 7.81 (t, J = 6.3 Hz, 1H), 7.46 (t, J = 7.6 Hz, 1H), 7.38 (t, J = 6.8 Hz, 1H), |
| | | | | | | 7.20 (dd, J = 11.9, 7.9 Hz, |
| | | | | | | 2H), 6.88 (d, J = 2.9 Hz, |
| | | | | | | 1H), 1.61 (q, J = 4.4 Hz, |
| | | | | | | 2H), 1.17 (dd, J = 7.7, 3.3 |
| | | | | | | Hz, 2H). |
| 138 | 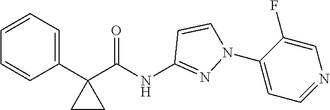 [SMILES: O=C(Nc1ccn(-c2ccncc2F)n1)C1(c2ccccc2)CC1] | F | 14 | 0.055 | 323.14 | 1H NMR (400 MHz, DMSO-d₆) δ 9.55 (s, 1H), 8.72 (d, J = 4.1 Hz, 1H), 8.45 (d, J = 5.4 Hz, 1H), 8.28 (d, J = 2.8 Hz, 1H), 7.80 (t, J = 6.3 Hz, 1H), 7.44 (d, J = 7.5 Hz, 2H), |
| | | | | | | 7.38 (t, J = 7.4 Hz, 2H), |
| | | | | | | 7.32 (t, J = 7.2 Hz, 1H), |
| | | | | | | 6.91 (d, J = 2.7 Hz, 1H), |
| | | | | | | 1.48 (q, J = 4.3 Hz, 2H), |
| | | | | | | 1.14 (q, J = 4.4 Hz, 2H). |
| 139 | 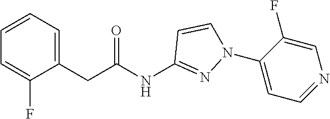 [SMILES: O=C(Cc1ccccc1F)Nc1ccn(-c2ccncc2F)n1] | F | 11 | 0.40 | 315.15 | 1H NMR (400 MHz, DMSO-d₆) δ 11.19 (s, 1H), 8.74 (d, J = 4.0 Hz, 1H), 8.50 (d, J = 5.3 Hz, 1H), 8.30 (d, J = 2.6 Hz, 1H), 7.84 (t, J = 6.3 Hz, 1H), 7.38 (dd, J = 8.6, 6.8 Hz, 1H), 7.31 (dd, J = 8.0, 5.8 |
| | | | | | | Hz, 1H), 7.22-7.07 (m, |
| | | | | | | 2H), 6.90 (d, J = 2.7 Hz, |
| | | | | | | 1H), 3.78 (s, 2H). |
| 140 | 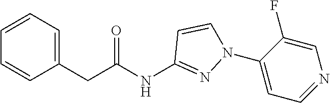 [SMILES: O=C(Cc1ccccc1)Nc1ccn(-c2ccncc2F)n1] | F | 16 | 1.1 | 297.11 | 1H NMR (400 MHz, DMSO-d₆) δ 11.15 (s, 1H), 8.73 (d, J = 4.0 Hz, 1H), 8.50 (d, J = 5.3 Hz, 1H), 8.29 (d, J = 2.6 Hz, 1H), 7.82 (t, J = 6.2 Hz, 1H), 7.42-7.17 (m, 5H), 6.91 |
| | | | | | | (d, J = 2.7 Hz, 1H), 3.67 (s, |
| | | | | | | 2H). |
| 141 | 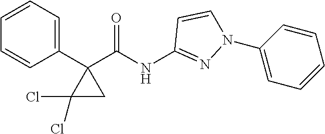 [SMILES: O=C(Nc1ccn(-c2ccccc2)n1)C1(c2ccccc2)CC1(Cl)Cl] | D | 36 | 0.34 | 372.08 | 1H NMR (400 MHz, DMSO-d₆) δ 10.01 (s, 1H), 8.40 (d, J = 2.4 Hz, 1H), 7.80-7.71 (m, 2H), 7.71- 7.65 (m, 2H), 7.51-7.32 (m, 5H), 7.27 (m, 1H), 6.74 (d, J = 2.4 Hz, 1H), 2.59 (d, J = 8.5 Hz, 1H), 2.33 (d, J = 8.5 Hz, 1H). |
| 142 | 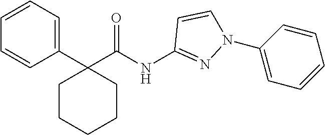 [SMILES: O=C(Nc1ccn(-c2ccccc2)n1)C1(c2ccccc2)CCCCC1] | D | 86 | >5 | 346.48 | 1H NMR (400 MHz, DMSO-d₆) δ 10.01 (s, 1H), 8.77-8.69 (m, 1H), 8.52 (dd, J = 8.4, 1.5 Hz, 1H), 8.37 (d, J = 2.4 Hz, 1H), 7.80-7.70 (m, 2H), 7.53- 7.47 (m, 1H), 7.45-7.20 (m, 5H), 6.81 (m, 1H), 2.60 (m, 2H), 2.33 (m, 2H), |
| | | | | | | 1.73 (m, 2H), 1.52-1.38 |
| | | | | | | (m, 2H), 1.24 (m, 2H). |
| 143 | 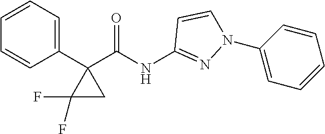 [SMILES: O=C(Nc1ccn(-c2ccccc2)n1)C1(c2ccccc2)CC1(F)F] | D | 55 | 0.14 | 340.39 | 1H NMR (400 MHz, DMSO-d₆) δ 11.16 (s, 1H), 8.41 (d, J = 2.4 Hz, 1H), 7.80-7.72 (m, 2H), 7.70- 7.61 (m, 2H), 7.56-7.33 (m, 4H), 7.27 (t, J = 7.4 Hz, 1H), 6.74 (d, J = 2.4 Hz, 1H), 2.47-2.37 (m, 1H), 2.20-2.08 (m, 1H). |
| 144 | 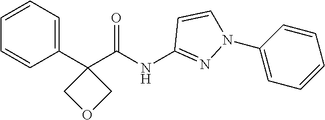 [SMILES: O=C(Nc1ccn(-c2ccccc2)n1)C1(c2ccccc2)COC1] | D | 54 | 0.26 | 320.19 | 1H NMR (400 MHz, DMSO-d₆) δ 10.93 (s, 1H), 8.41 (d, J = 2.4 Hz, 1H), 7.80-7.70 (m, 2H), 7.51- 7.37 (m, 6H), 7.29 (m, 2H), 6.82 (d, J = 2.4 Hz, 1H), 5.20 (d, J = 6.6 Hz, 2H), 4.87 (d, J = 6.6 Hz, 2H). |
| 145 | 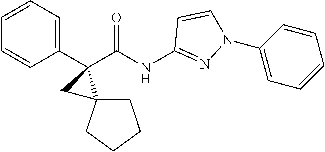 [SMILES: O=C(Nc1ccn(-c2ccccc2)n1)[C@]1(c2ccccc2)CC12CCCC2] | D | 19 | >5 | 358.47 | 1H NMR (400 MHz, DMSO-d₆) δ 10.10 (s, 1H), 8.37 (m, 1H), 7.77-7.68 (m, 2H), 7.63-7.51 (m, 2H), 7.46 (m, 2H), 7.36 (m, 2H), 7.30-7.23 (m, 2H), 6.80-6.69 (m, 1H), 1.93 (m, 1H), 1.83-1.57 (m, 5H), 1.46 (m, 2H), 1.24 (m, 1H), 0.98 (m, 1H). |
| | | | | | | [α]_{D} −16.2 |
| 146 | 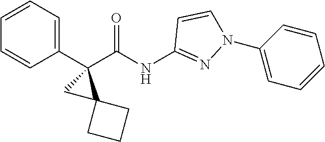 [SMILES: O=C(Nc1ccn(-c2ccccc2)n1)[C@@]1(c2ccccc2)CC12CCC2] | D | 36 | 0.087 | 344.50 | 1H NMR (400 MHz, DMSO-d₆) δ 9.37 (s, 1H), 8.38 (d, J = 2.5 Hz, 1H), 7.81-7.63 (m, 2H), 7.53- 7.37 (m, 6H), 7.36-7.19 (m, 2H), 6.76 (d, J = 2.5 Hz, 1H), 2.55-2.52 (m, 1H), 2.17-1.90 (m, 4H), 1.74 (d, J = 5.1 Hz, 1H), 1.66 (s, |
| | | | | | | 1H), 1.31 (d, J = 5.1 Hz, |
| | | | | | | 1H). |
| | | | | | | [α]_{D} −48.0 |
| 147 | 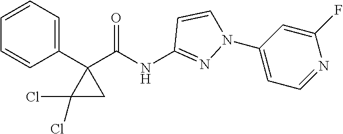 [SMILES: O=C(Nc1ccn(-c2ccnc(F)c2)n1)C1(c2ccccc2)CC1(Cl)Cl] | D | 6 | 0.28 | 391.09 | 1H NMR (400 MHz, DMSO-d₆) δ 11.38 (s, 1H), 8.66 (m, 1H), 8.29 (m, 1H), 7.74 (m, 1H), 7.72-7.65 (m, 2H), 7.52 (m, 1H), 7.45- 7.33 (m, 3H), 6.88 (d, J = 2.8 Hz, 1H), 2.59 (d, J = 8.6 Hz, 1H), 2.36 (d, J = 8.6 Hz, 1H). |
| 148 | 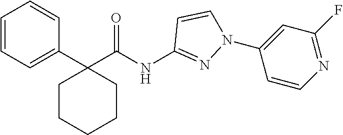 [SMILES: O=C(Nc1ccn(-c2ccnc(F)c2)n1)C1(c2ccccc2)CCCCC1] | D | 12 | >5 | 365.42 | 1H NMR (400 MHz, DMSO-d₆) δ 10.20 (s, 1H), 8.64 (d, J = 2.7 Hz, 1H), 8.26 (m, 1H), 7.72 (m, 1H), 7.49 (s, 1H), 7.48-7.41 (m, 2H), 7.35 (m, 2H), 7.24 (m, 1H), 6.96 (d, J = 2.7 Hz, 1H), 2.63-2.57 (m, 2H), 1.82-1.19 (m, 8H). |
| 149 | 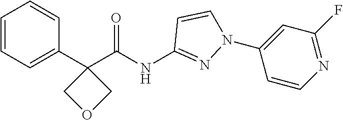 [SMILES: O=C(Nc1ccn(-c2ccnc(F)c2)n1)C1(c2ccccc2)COC1] | D | 46 | 0.66 | 339.2 | 1H NMR (400 MHz, DMSO-d₆) δ 11.09 (s, 1H), 8.68 (d, J = 2.7 Hz, 1H), 8.28 (d, J = 5.7 Hz, 1H), 7.72 (d, J = 5.7 Hz, 1H), 7.53-7.46 (m, 3H), 7.42 (m, 2H), 7.36-7.29 (m, 1H), 6.97 (d, J = 2.7 Hz, |
| | | | | | | 1H), 5.20 (d, J = 6.6 Hz, |
| | | | | | | 2H), 4.88 (d, J = 6.6 Hz, |
| | | | | | | 2H). |
| 150 | 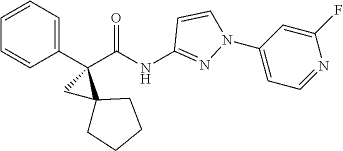 [SMILES: O=C(Nc1ccn(-c2ccnc(F)c2)n1)[C@@]1(c2ccccc2)CC12CCCC2] | D | 6 | <0.05 | 377.22 | 1H NMR (400 MHz, DMSO-d₆) δ 10.33 (s, 1H), 8.63 (d, J = 2.7 Hz, 1H), 8.26 (d, J = 5.8 Hz, 1H), 7.72 (d, J = 5.8 Hz, 1H), 7.60-7.52 (m, 2H), 7.50 (m, 1H), 7.41-7.18 (m, 5H), 6.89 (d, J = 2.7 Hz, 1H), 1.92 (m, 1H), 1.58 (m, 5H), 1.29-0.70 (m, 4H). |
| | | | | | | [α]_{D} +21.2 |
| 151 | 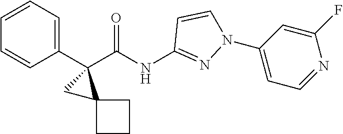 [SMILES: O=C(Nc1ccn(-c2ccnc(F)c2)n1)[C@@]1(c2ccccc2)CC12CCC2] | D | 13 | <0.05 | 363.18 | 1H NMR (400 MHz, DMSO-d₆) δ 9.64 (s, 1H), 8.64 (d, J = 2.7 Hz, 1H), 8.25 (m, 1H), 7.76-7.67 (m, 1H), 7.49-7.38 (m, 4H), 7.35-7.29 (m, 1H), 6.91 (d, J = 2.7 Hz, 1H), 2.45 (s, 1H), 2.17-1.85 (m, 3H), 1.75 (d, J = 5.2 |
| | | | | | | Hz, 1H), 1.66 (d, J = 10.0 |
| | | | | | | Hz, 1H), 1.34 (d, J = 5.2 Hz, |
| | | | | | | 1H). |
| | | | | | | [α]_{D} +44.8 |
| 152 | 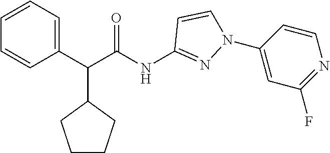 [SMILES: O=C(Nc1ccn(-c2ccnc(F)c2)n1)C(c1ccccc1)C1CCCC1] | D | 32 | 1.7 | 365.23 | 1H NMR (400 MHz, DMSO-d₆) δ 11.05 (s, 1H), 8.63 (m, 1H), 8.28 (d, J = 5.8 Hz, 1H), 7.71 (d, J = 5.7 Hz, 1H), 7.49 (m, 1H), 7.41 (m, 2H), 7.32 (m, 2H), 7.23 (m, 1H), 6.99-6.85 (m, 1H), 3.53 (d, J = 11.1 Hz, 1H), 1.82-0.89 (m, 10H). |
| 153 | 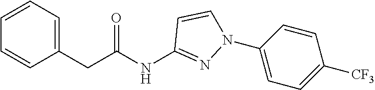 [SMILES: O=C(Cc1ccccc1)Nc1ccn(-c2ccc(C(F)(F)F)cc2)n1] | B | 18 | >5 | 345.9 | 1H NMR (400 MHz, DMSO-d₆) δ 11.05 (s, 1H), 8.52 (d, J = 2.7 Hz, 1H), 7.97 (d, J = 8.5 Hz, 2H), 7.84 (d, J = 8.5 Hz, 2H), 7.42-7.16 (m, 6H), 6.82 |
| | | | | | | (d, J = 2.6 Hz, 1H), 3.66 (s, |
| | | | | | | 2H). |
| 154 | 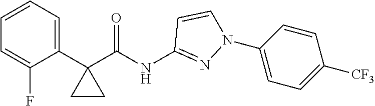 [SMILES: O=C(Nc1ccn(-c2ccc(C(F)(F)F)cc2)n1)C1(c2ccccc2F)CC1] | B | 5 | 0.36 | 389.9 | 1H NMR (400 MHz, DMSO-d₆) δ 9.47 (s, 1H), 8.50 (d, J = 2.7 Hz, 1H), 7.94 (d, J = 8.5 Hz, 2H), 7.81 (d, J = 8.5 Hz, 2H), 7.47 (t, J = 7.3 Hz, 1H), 7.39 (q, J = 7.0 Hz, 1H), |
| | | | | | | 7.20 (dd, J = 11.6, 8.0 Hz, |
| | | | | | | 2H), 6.79 (d, J = 2.6 Hz, |
| | | | | | | 1H), 1.21 (dt, J = 7.5, 4.0 |
| | | | | | | Hz, 2H), 1.15 (q, J = 4.3 Hz, |
| | | | | | | 2H). |
| 155 | 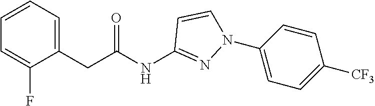 [SMILES: O=C(Cc1ccccc1F)Nc1ccn(-c2ccc(C(F)(F)F)cc2)n1] | B | 14 | >5 | 363.83 | 1H NMR (400 MHz, DMSO-d₆) δ 11.10 (s, 1H), 8.57 (d, J = 2.6 Hz, 1H), 8.00 (d, J = 8.6 Hz, 2H), 7.87 (d, J = 8.7 Hz, 2H), 7.41-7.29 (m, 2H), 7.22- 7.13 (m, 2H), 6.83 (d, J = 2.6 Hz, 1H), 3.77 (s, 2H). |
| 156 | 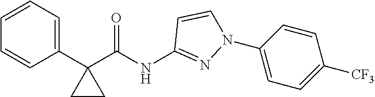 [SMILES: O=C(Nc1ccn(-c2ccc(C(F)(F)F)cc2)n1)C1(c2ccccc2)CC1] | B | 12 | >2 | 371.81 | 1H NMR (400 MHz, DMSO-d₆) δ 9.38 (s, 1H), 8.55 (d, J = 2.7 Hz, 1H), 7.95 (d, J = 8.5 Hz, 2H), 7.82 (d, J = 8.6 Hz, 2H), 7.50-7.44 (m, 2H), 7.40 (t, J = 7.5 Hz, 2H), 7.36- |
| | | | | | | 7.31 (m, 1H), 6.83 (d, J = |
| | | | | | | 2.7 Hz, 1H), 1.49 (q, J = 4.2 |
| | | | | | | Hz, 2H), 1.14 (q, J = 4.3 Hz, |
| | | | | | | 2H). |
| 157 | 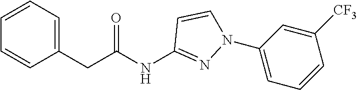 [SMILES: O=C(Cc1ccccc1)Nc1ccn(-c2cccc(C(F)(F)F)c2)n1] | B | 5 | >5 | 345.77 | 1H NMR (400 MHz, DMSO-d₆) δ 11.05 (s, 1H), 8.58 (d, J = 2.7 Hz, 1H), 8.15-8.05 (m, 2H), 7.73 (t, J = 7.9 Hz, 1H), 7.62 (d, J = 8.0 Hz, 1H), 7.38-7.20 |
| | | | | | | (m, 5H), 6.82 (d, J = 2.6 |
| | | | | | | Hz, 1H), 3.66 (s, 2H). |
| 158 | 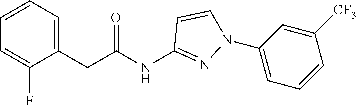 [SMILES: O=C(Cc1ccccc1F)Nc1ccn(-c2cccc(C(F)(F)F)c2)n1] | B | 24 | 2.9 | 363.87 | 1H NMR (400 MHz, DMSO-d₆) δ 11.09 (s, 1H), 8.60 (d, J = 2.7 Hz, 1H), 8.11 (dd, J = 11.1, 2.9 Hz, 2H), 7.74 (t, J = 7.9 Hz, 1H), 7.63 (d, J = 7.8 Hz, 1H), 7.35 (dtd, J = 30.6, |
| | | | | | | 7.4, 1.7 Hz, 2H), 7.22- |
| | | | | | | 7.14 (m, 2H), 6.81 (d, J = |
| | | | | | | 2.6 Hz, 1H), 3.77 (s, 2H). |
| 159 | 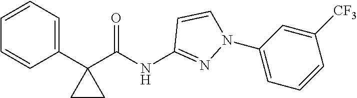 [SMILES: O=C(Nc1ccn(-c2cccc(C(F)(F)F)c2)n1)C1(c2ccccc2)CC1] | B | 16 | >5 | 371.86 | 1H NMR (400 MHz, DMSO-d₆) δ 9.48 (s, 1H), 8.58 (d, J = 2.6 Hz, 1H), 8.06 (d, J = 8.7 Hz, 2H), 7.69 (t, J = 7.8 Hz, 1H), 7.60 (d, J = 7.8 Hz, 1H), 7.47 7.25 (m, 5H), 6.82 |
| | | | | | | (d, J = 2.6 Hz, 1H), 1.48 (q, |
| | | | | | | J = 4.2 Hz, 2H), 1.13 (q, J = |
| | | | | | | 4.3 Hz, 2H). |
| 160 | 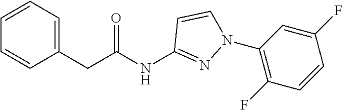 [SMILES: O=C(Cc1ccccc1)Nc1ccn(-c2cc(F)ccc2F)n1] | B | 46 | >5 | 313.86 | 1H NMR (400 MHz, DMSO-d₆) δ 11.11 (s, 1H), 7.77 (dd, J = 13.5, 2.1 Hz, 1H), 7.30 (qq, J = 11.9, 6.2, 5.8 Hz, 6H), 7.08 (ddt, J = 1H), 7.30 (qq, J = 11.9, 6.2, 5.8 Hz, 6H), 7.08 (ddt, J = |
| | | | | | | 9.0, 6.6, 3.5 Hz, 2H), 4.60 |
| | | | | | | (d, J = 13.5 Hz, 1H), 3.57 |
| | | | | | | (s, 2H). |
| 161 | 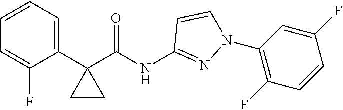 [SMILES: O=C(Nc1ccn(-c2cc(F)ccc2F)n1)C1(c2ccccc2F)CC1] | B | 6 | 0.98 | 357.75 | 1H NMR (400 MHz, DMSO-d₆) δ 9.49 (s, 1H), 8.13 (t, J = 2.7 Hz, 1H), 7.63-7.44 (m, 3H), 7.40 (tdd, J = 7.5, 5.2, 1.8 Hz, 1H), 7.21 (dq, J = 8.0, 4.2, 3.4 Hz, 3H), 6.80 (d, J = 2.6 Hz, 1H), 1.60 (q, J = 4.3 Hz, |
| | | | | | | 2H), 1.16 (q, J = 4.3 Hz, |
| | | | | | | 2H). |
| 162 | 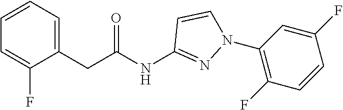 [SMILES: O=C(Cc1ccccc1F)Nc1ccn(-c2cc(F)ccc2F)n1] | B | 12 | 0.74 | 331.86 | 1H NMR (400 MHz, DMSO-d₆) δ 11.08 (s, 1H), 8.16 (t, J = 2.6 Hz, 1H), 7.57 (m, J = 14.1, 11.4, 7.7, 4.1 Hz, 2H), 7.42- 7.12 (m, 5H), 6.82 (d, J = 2.6 Hz, 1H), 3.76 (s, 2H). |
| 163 | 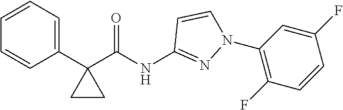 [SMILES: O=C(Nc1ccn(-c2cc(F)ccc2F)n1)C1(c2ccccc2)CC1] | B | 28 | 4.5 | 339.89 | 1H NMR (400 MHz, DMSO-d₆) δ 9.37 (s, 1H), 8.14 (t, J = 2.7 Hz, 1H), 7.61-7.27 (m, 7H), 7.22 (ddd, J = 8.8, 6.4, 3.5 Hz, 1H), 6.82 (d, J = 2.6 Hz, 1H), 1.48 (q, J = 4.2 Hz, |
| | | | | | | 2H), 1.14 (q, J = 4.3 Hz, |
| | | | | | | 2H). |
| 164 | 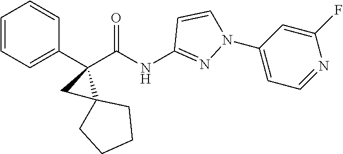 [SMILES: O=C(Nc1ccn(-c2ccnc(F)c2)n1)[C@]1(c2ccccc2)CC12CCCC2] | D | 3 | >5 | 376.94 | 1H NMR (400 MHz, DMSO-d₆) δ 10.34 (s, 1H), 8.63 (d, J = 2.8 Hz, 1H), 8.27 (m, 1H), 7.72 (m, 1H), 7.58-7.46 (m, 3H), 7.32 (m, 3H), 6.90 (d, J = 2.8 Hz, 1H), 1.92 (m, 1H), 1.79-1.59 (m, 5H), 1.47 (m, 2H), 1.26 (m, 1H), 1.03- 0.89 (m, 1H). |
| | | | | | | [α]_{D} −7.3 |
| 165 | 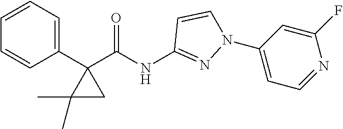 [SMILES: CC1(C)CC1(C(=O)Nc1ccn(-c2ccnc(F)c2)n1)c1ccccc1] | D | 7 | 0.050 | 350.90 | 1H NMR (400 MHz, DMSO-d₆) δ 10.63 (s, 1H), 8.61 (d, J = 2.8 Hz, 1H), 8.27 (m, 1H), 7.73 (m, 1H), 7.61 (m, 2H), 7.55-7.46 (m, 1H), 7.41-7.19 (m, 3H), 6.88 (d, J = 2.8 Hz, 1H), 1.41 (d, J = 5.3 Hz, 1H), 1.22 (s, 3H), 1.05 (d, |
| | | | | | | J = 5.3 Hz, 1H), 0.85 (s, 3H). |
| 166 | 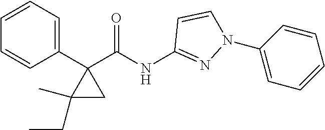 [SMILES: CCC1(C)CC1(C(=O)Nc1ccn(-c2ccccc2)n1)c1ccccc1] | D | 36 | 0.63 | 345.90 | 1H NMR (400 MHz, DMSO-d₆) δ 10.48 (s, 1H), 8.36 (d, J = 2.5 Hz, 1H), 7.81-7.69 (m, 2H), 7.63- 7.57 (m, 2H), 7.46 (m, 2H), 7.37-7.18 (m, 4H), 6.73 (m, 1H), 1.33 (m, 1H), 1.17 (s, 3H), 1.06-0.97 (m, 2H), 0.83 (m, 3H), 0.73- |
| | | | | | | 0.58 (m, 1H). |
| 167 | 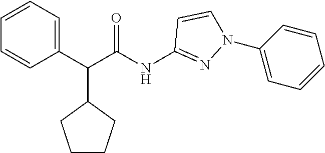 [SMILES: O=C(Nc1ccn(-c2ccccc2)n1)C(c1ccccc1)C1CCCC1] | D | 31 | >5 | 345.9 | 1H NMR (400 MHz, DMSO-d₆) δ 10.89 (s, 1H), 8.37 (d, J = 2.6 Hz, 1H), 7.74 (m, 2H), 7.50-7.39 (m, 4H), 7.37-7.17 (m, 4H), 6.77 (d, J = 2.6 Hz, 1H), 3.51 (m, 1H), 2.62 (m, 1H), 1.86-1.16 (m, 7H), 0.95 (m, 1H). |
| 168 | 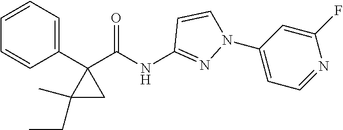 [SMILES: CCC1(C)CC1(C(=O)Nc1ccn(-c2ccnc(F)c2)n1)c1ccccc1] | D | 10 | 0.086 | 364.94 | 1H NMR (400 MHz, DMSO-d₆) δ 10.69 (s, 1H), 8.61 (d, J = 2.8 Hz, 1H), 8.27 (m, 1H), 7.73 (m, 1H), 7.63-7.55 (m, 2H), 7.50 (m, 1H), 7.29 (m, 3H), 6.88 (d, J = 2.8 Hz, 1H), 1.37- 1.28 (m, 2H), 1.16 (s, 3H), 1.07 (d, J = 5.4 Hz, 1H), |
| | | | | | | 0.83 (m, 3H), 0.66 (m, 1H). |
| 169 | 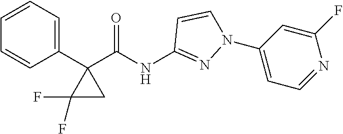 [SMILES: O=C(Nc1ccn(-c2ccnc(F)c2)n1)C1(c2ccccc2)CC1(F)F] | D | 18 | 0.052 | 358.84 | 1H NMR (400 MHz, DMSO-d₆) δ 11.35 (s, 1H), 8.66 (d, J = 2.8 Hz, 1H), 8.29 (d, J = 5.8 Hz, 1H), 7.74 (d, J = 5.8 Hz, 1H), 7.64 (m, 2H), 7.52 (m, 1H), 7.41 (m, 3H), 6.88 (d, J = 2.8 Hz, 1H), 2.48-2.37 (m, 1H), 2.17 (m, 1H). |
| 170 | 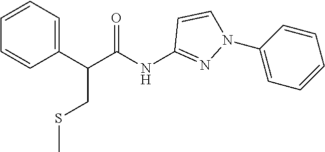 [SMILES: CSCC(C(=O)Nc1ccn(-c2ccccc2)n1)c1ccccc1] | D | 48 | >5 | 338.20 | 1H NMR (400 MHz, DMSO-d₆) δ 11.04 (s, 1H), 8.38 (d, J = 2.6 Hz, 1H), 7.74 (m, 2H), 7.47 (m, 2H), 7.42 (m, 2H), 7.34 (m, 2H), 7.27 (m, 2H), 6.79 (d, J = 2.6 Hz, 1H), 4.14-4.05 (m, 3H), 3.22 (m, 1H), 2.75 (m, 1H). |
| 171 | 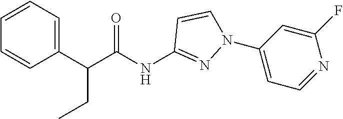 [SMILES: CCC(C(=O)Nc1ccn(-c2ccnc(F)c2)n1)c1ccccc1] | D | 46 | 0.14 | 325.21 | 1H NMR (400 MHz, DMSO-d₆) δ 11.09 (s, 1H), 8.63 (d, J = 2.7 Hz, 1H), 8.28 (d, J = 5.7 Hz, 1H), 7.71 (d, J = 5.7 Hz, 1H), 7.49 (m, 1H), 7.39 (m, 2H), 7.33 (m, 2H), 7.24 (m, 1H), 6.93 (d, J = 2.7 Hz, 1H), |
| | | | | | | 2.06 (m, 1H), 1.70 (m, 1H), |
| | | | | | | 0.85 (t, J = 7.3 Hz, 3H) |
| 172 | 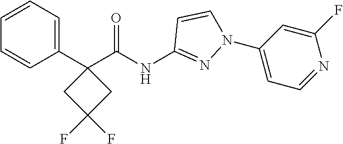 [SMILES: O=C(Nc1ccn(-c2ccnc(F)c2)n1)C1(c2ccccc2)CC(F)(F)C1] | D | 49 | 0.38 | 373.15 | 1H NMR (400 MHz, DMSO-d₆) δ 11.01 (s, 1H), 8.63 (d, J = 2.7 Hz, 1H), 8.27 (m, 1H), 7.72 (m, 1H), 7.57-7.48 (m, 3H), 7.41 (m, 2H), 7.31 (m, 1H), 6.90 (d, J = 2.7 Hz, 1H), 4.13 (m, 2H), 3.13 (m, 2H). |
| 173 | 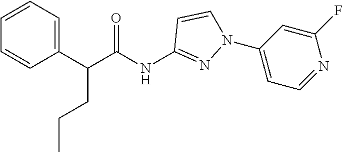 [SMILES: CCCC(C(=O)Nc1ccn(-c2ccnc(F)c2)n1)c1ccccc1] | D | 48 | 0.072 | 339.25 | 1H NMR (400 MHz, DMSO-d₆) δ 11.09 (s, 1H), 8.62 (d, J = 2.7 Hz, 1H), 8.28 (d, J = 5.7 Hz, 1H), 7.71 (d, J = 5.7 Hz, 1H), 7.48 (m, 1H), 7.39 (m, 2H), 7.33 (m, 2H), 7.24 (m, 1H), 6.92 (d, J = 2.7 Hz, 1H), 3.81 (m, 1H), 2.12-1.96 (m, 1H), 1.65 (m, 1H), 1.23 |
| | | | | | | (m, 2H), 0.90 (t, J = 7.3 Hz, |
| | | | | | | 3H). |
| 174 | 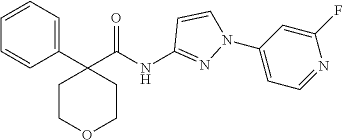 [SMILES: O=C(Nc1ccn(-c2ccnc(F)c2)n1)C1(c2ccccc2)CCOCC1] | D | 7 | >5 | 367.22 | 1H NMR (400 MHz, DMSO-d₆) δ 10.40 (s, 1H), 8.62 (d, J = 2.7 Hz, 1H), 8.25 (d, J = 5.7 Hz, 1H), 7.71 (d, J = 5.7 Hz, 1H), 7.48 (s, 1H), 7.44 (m, 2H), 7.38 (m, 2H), 7.27 (m, 1H), 6.95 (d, J = 2.7 Hz, 1H), 3.85-3.75 (m, 2H), 3.67- |
| | | | | | | 3.35 (m, 1H), 2.64 (m, 2H), |
| | | | | | | 1.96 (m, 2H), 1.37-1.12 |
| | | | | | | (m, 1H). |
| 175 | 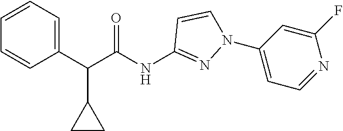 [SMILES: O=C(Nc1ccn(-c2ccnc(F)c2)n1)C(c1ccccc1)C1CC1] | D | 40 | 0.52 | 337.21 | 1H NMR (400 MHz, DMSO-d₆) δ 10.90 (s, 1H), 8.52 (d, J = 2.7 Hz, 1H), 8.16 (d, J = 5.7 Hz, 1H), 7.60 (d, J = 5.7 Hz, 1H), 7.38 (m, 1H), 7.31 (m, 2H), 7.22 (m, 2H), 7.14 (m, 1H), 6.83 (d, J = 2.7 Hz, 1H), 2.93-2.82 (m, 1H), 1.38 |
| | | | | | | (m, 1H), 0.43 (m, 2H), 0.25 |
| | | | | | | (m, 1H), −0.01 (m, 1H). |
| 176 | 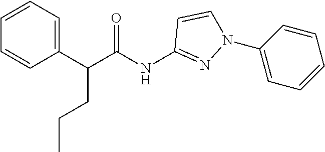 [SMILES: CCCC(C(=O)Nc1ccn(-c2ccccc2)n1)c1ccccc1] | D | 53 | 0.87 | 320.26 | 1H NMR (400 MHz, DMSO-d₆) δ 10.92 (s, 1H), 8.37 (d, J = 2.6 Hz, 1H), 7.73 (m, 2H), 7.47 (m, 2H), 7.40 (m, 2H), 7.32 (m, 2H), 7.25 (m, 2H), 6.78 (d, J = 2.6 Hz, 1H), 3.79 (m, 1H), 2.04 (m, 1H), 1.73-1.57 (m, 1H), 1.25 (m, 2H), 0.90 (m, 3H). |
| 177 | 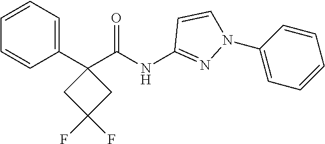 [SMILES: O=C(Nc1ccn(-c2ccccc2)n1)C1(c2ccccc2)CC(F)(F)C1] | D | 59 | 0.79 | 354.22 | 1H NMR (400 MHz, DMSO-d₆) δ 10.84 (s, 1H), 8.37 (d, J = 2.5 Hz, 1H), 7.74 (m, 2H), 7.53 (m, 2H), 7.47 (m, 2H), 7.41 (m, 2H), 7.28 (m, 2H), 6.76 (d, J = 2.5 Hz, 1H), 3.58-3.42 (m, 2H), 3.12 (m, 2H). |
| 178 | 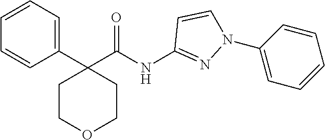 [SMILES: O=C(Nc1ccn(-c2ccccc2)n1)C1(c2ccccc2)CCOCC1] | D | 36 | >5 | 348.22 | 1H NMR (400 MHz, DMSO-d₆) δ 10.23 (s, 1H), 8.37 (d, J = 2.5 Hz, 1H), 7.73 (m, 2H), 7.47-7.42 (m, 3H), 7.38 (m, 2H), 7.26 (m, 2H), 6.80 (d, J = 2.5 Hz, 1H), 3.86-3.72 (m, 2H), 3.45 (m, 2H), 2.66 (m, 2H), 1.95 (m, 2H). |
| 179 | 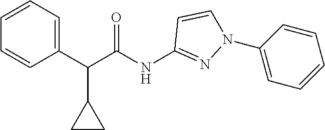 [SMILES: O=C(Nc1ccn(-c2ccccc2)n1)C(c1ccccc1)C1CC1] | D | 52 | 1.6 | 318.22 | 1H NMR (400 MHz, DMSO-d₆) δ 10.75 (s, 1H), 8.27 (d, J = 2.6 Hz, 1H), 7.64 (m, 2H), 7.40-7.28 (m, 4H), 7.19 (m, 4H), 6.69 (m, 1H), 2.94-2.84 (m, 1H), 1.40 (m, 1H), 0.43 (m, 2H), 0.27-0.21 (m, 1H), −0.01 (m, 1H). |
| 180 | 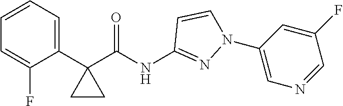 [SMILES: O=C(Nc1ccn(-c2cncc(F)c2)n1)C1(c2ccccc2F)CC1] | N | 63 | 0.020 | 341.01 | 1H NMR (300 MHz, DMSO-d₆) δ 9.50 (s, 1H), 8.92 (t, J = 1.8 Hz, 1H), 8.52 (d, J = 2.7 Hz, 1H), 8.47 (d, J = 2.5 Hz, 1H), 8.10 (dt, J = 10.5, 2.4 Hz, 1H), 7.55-7.33 (m, 2H), |
| | | | | | | 7.28-7.13 (m, 2H), 6.83 |
| | | | | | | (d, J = 2.6 Hz, 1H), 1.61 (q, |
| | | | | | | J = 4.3 Hz, 2H), 1.16 (q, J = |
| | | | | | | 4.3 Hz, 2H). |
| 181 | 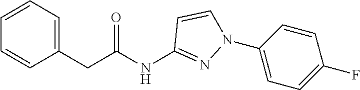 [SMILES: O=C(Cc1ccccc1)Nc1ccn(-c2ccc(F)cc2)n1] | B | 36 | 0.25 | 295.90 | 1H NMR (400 MHz, DMSO-d₆) δ 10.97 (s, 1H), 8.35 (d, J = 2.6 Hz, 1H), 7.78 (dd, J = 9.0, 4.6 Hz, 2H), 7.43-7.13 (m, 7H), 6.75 (d, J = 2.5 Hz, 1H), 3.65 (s, 2H). |
| 182 | 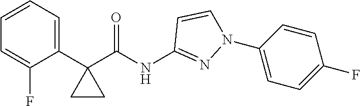 [SMILES: O=C(Nc1ccn(-c2ccc(F)cc2)n1)C1(c2ccccc2F)CC1] | F | 16 | 0.074 | 340.02 | 1H NMR (300 MHz, DMSO-d₆) δ 9.26 (s, 1H), 8.33 (d, J = 2.5 Hz, 1H), 7.84-7.68 (m, 2H), 7.48 (t, J = 7.7 Hz, 1H), 7.40 (d, J = 5.7 Hz, 1H), 7.25 t(m, 4H), 6.71 (d, J = 2.5 Hz, |
| | | | | | | 1H), 1.60 (d, J = 2.6 Hz, |
| | | | | | | 2H), 1.15 (d, J = 2.7 Hz, |
| | | | | | | 2H). |
| 183 | 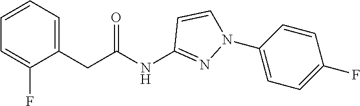 [SMILES: O=C(Cc1ccccc1F)Nc1ccn(-c2ccc(F)cc2)n1] | B | 31 | 0.17 | 313.87 | 1H NMR (400 MHz, DMSO-d₆) δ 11.00 (s, 1H), 8.36 (d, J = 2.6 Hz, 1H), 7.79 (dd, J = 9.0, 4.7 Hz, 2H), 7.45-7.26 (m, 4H), 7.24-7.13 (m, 2H), 6.74 (d, J = 2.4 Hz, 1H), 3.76 (s, |
| | | | | | | 2H). |
| 184 | 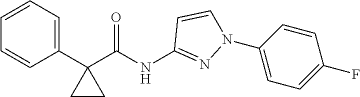 [SMILES: O=C(Nc1ccn(-c2ccc(F)cc2)n1)C1(c2ccccc2)CC1] | B | 26 | 0.28 | 321.92 | 1H NMR (400 MHz, DMSO-d₆) δ 9.20 (s, 1H), 8.35 (d, J = 2.6 Hz, 1H), 7.79-7.68 (m, 2H), 7.46 (d, J = 7.5 Hz, 2H), 7.40 (t, J = 7.4 Hz, 2H), 7.37-7.24 (m, 3H), 6.75 (d, J = 2.5 |
| | | | | | | Hz, 1H), 1.48 (q, J = 4.2 Hz, |
| | | | | | | 2H), 1.13 (q, J = 4.3 Hz, |
| | | | | | | 2H). |
| 185 | 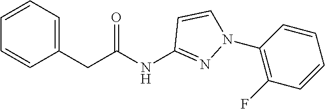 [SMILES: O=C(Cc1ccccc1)Nc1ccn(-c2ccccc2F)n1] | B | 33 | 0.47 | 295.93 | 1H NMR (400 MHz, DMSO-d₆) δ 11.00 (s, 1H), 8.17-7.92 (m, 1H), 7.73 (td, J = 7.9, 2.2 Hz, 1H), 7.54-7.14 (m, 8H), 6.78 (d, J = 2.6 Hz, 1H), 3.65 (s, 2H). |
| 186 | 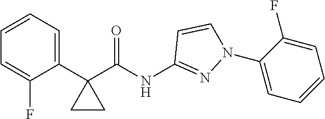 [SMILES: O=C(Nc1ccn(-c2ccccc2F)n1)C1(c2ccccc2F)CC1] | R | 8 | 0.14 | 339.88 | 1H NMR (400 MHz, DMSO-d₆) δ 9.38 (s, 1H), 8.06 (t, J = 2.7 Hz, 1H), 7.69 (td, J = 8.0, 1.8 Hz, 1H), 7.58-7.28 (m, 5H), 7.28-7.09 (m, 2H), 6.75 (d, J = 2.6 Hz, 1H), 1.59 (q, J = 4.3 Hz, 2H), 1.14 (q, J = |
| | | | | | | 4.3 Hz, 2H). |
| 187 | 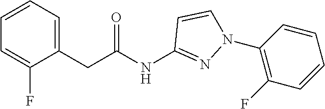 [SMILES: O=C(Cc1ccccc1F)Nc1ccn(-c2ccccc2F)n1] | B | 43 | 0.52 | 313.93 | 1H NMR (400 MHz, DMSO-d₆) δ 11.03 (s, 1H), 8.15-7.91 (m, 1H), 7.74 (td, J = 7.9, 2.3 Hz, 1H), 7.54-7.24 (m, 5H), 7.24- 7.05 (m, 2H), 6.77 (d, J = 2.6 Hz, 1H), 3.74 (s, 2H). |
| 188 | 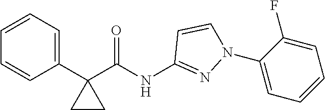 [SMILES: O=C(Nc1ccn(-c2ccccc2F)n1)C1(c2ccccc2)CC1] | B | 1 | 0.14 | 321.92 | 1H NMR (400 MHz, DMSO-d₆) δ 9.31 (s, 1H), 8.08 (t, J = 2.6 Hz, 1H), 7.68 (td, J = 8.1, 1.7 Hz, 1H), 7.47-7.27 (m, 8H), 6.79 (d, J = 2.5 Hz, 1H), 1.48 (q, J = 4.1 Hz, 2H), 1.12 (q, J = 4.2 Hz, 2H). |
| 189 | 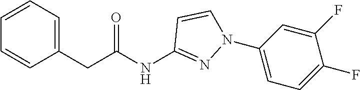 [SMILES: O=C(Cc1ccccc1)Nc1ccn(-c2ccc(F)c(F)c2)n1] | B | 66 | 0.23 | 313.93 | 1H NMR (400 MHz, DMSO-d₆) δ 10.99 (s, 1H), 8.40 (d, J = 2.6 Hz, 1H), 7.84 (ddd, J = 12.2, 7.0, 2.4 Hz, 1H), 7.69-7.43 (m, 2H), 7.41-7.15 (m, |
| | | | | | | 5H), 6.77 (d, J = 2.6 Hz, |
| | | | | | | 1H), 3.65 (s, 2H). |
| 190 | 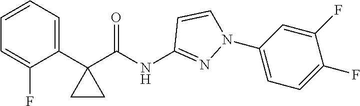 [SMILES: O=C(Nc1ccn(-c2ccc(F)c(F)c2)n1)C1(c2ccccc2F)CC1] | B | 2 | 0.15 | 357.92 | 1H NMR (400 MHz, DMSO-d₆) δ 9.37 (s, 1H), 8.38 (d, J = 2.6 Hz, 1H), 7.82 (ddd, J = 12.2, 7.0, 2.6 Hz, 1H), 7.66-7.32 (m, 4H), 7.20 (dd, J = 11.6, 7.9 Hz, 2H), 6.74 (d, J = 2.6 Hz, 1H), 1.58 (q, J = 4.3 Hz, |
| | | | | | | 2H), 1.14 (q, J = 4.4 Hz, |
| | | | | | | 2H). |
| 191 | 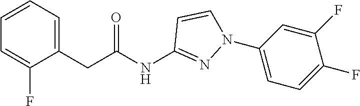 [SMILES: O=C(Cc1ccccc1F)Nc1ccn(-c2ccc(F)c(F)c2)n1] | B | 42 | 0.24 | 331.89 | 1H NMR (400 MHz, DMSO-d₆) δ 11.02 (s, 1H), 8.41 (d, J = 2.6 Hz, 1H), 7.85 (ddd, J = 12.2, 6.9, 2.5 Hz, 1H), 7.68-7.45 (m, 2H), 7.42-7.33 (m, 1H), 7.31 (dd, J = 7.8, 5.7 |
| | | | | | | Hz, 1H), 7.23-7.08 (m, |
| | | | | | | 2H), 6.76 (d, J = 2.6 Hz, |
| | | | | | | 1H), 3.75 (s, 2H). |
| 192 | 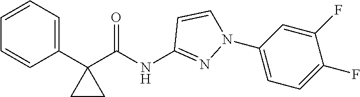 [SMILES: O=C(Nc1ccn(-c2ccc(F)c(F)c2)n1)C1(c2ccccc2)CC1] | B | 6 | 0.60 | 339.94 | 1H NMR (400 MHz, DMSO-d₆) δ 9.24 (s, 1H), 8.38 (d, J = 2.6 Hz, 1H), 7.82 (ddd, J = 12.2, 7.0, 2.5 Hz, 1H), 7.62-7.47 (m, 2H), 7.47-7.35 (m, 4H), 7.33 (t, J = 7.3 Hz, |
| | | | | | | 1H), 6.76 (d, J = 2.5 Hz, |
| | | | | | | 1H), 1.47 (q, J = 4.3 Hz, |
| | | | | | | 2H), 1.12 (q, J = 4.4 Hz, |
| | | | | | | 2H). |
| 193 | 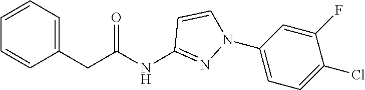 [SMILES: O=C(Cc1ccccc1)Nc1ccn(-c2ccc(Cl)c(F)c2)n1] | B | 47 | >5 | 329.85 | 1H NMR (400 MHz, DMSO-d₆) δ 11.02 (s, 1H), 8.45 (d, J = 2.7 Hz, 1H), 7.83 (dd, J = 11.0, 2.4 Hz, 1H), 7.74-7.57 (m, 2H), 7.41-7.28 (m, 4H), 7.24 (td, J = 5.7, 2.8 Hz, 1H), |
| | | | | | | 6.79 (d, J = 2.6 Hz, 1H), |
| | | | | | | 3.65 (s, 2H). |
| 194 | 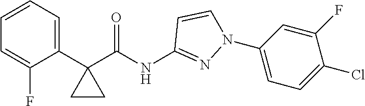 [SMILES: O=C(Nc1ccn(-c2ccc(Cl)c(F)c2)n1)C1(c2ccccc2F)CC1] | R | 15 | >5 | 373.89 | 1H NMR (400 MHz, DMSO-d₆) δ 9.43 (s, 1H), 8.44 (d, J = 2.7 Hz, 1H), 7.82 (dd, J = 11.1, 2.3 Hz, 1H), 7.71-7.55 (m, 2H), 7.46 (t, J = 7.4 Hz, 1H), 7.40 (q, J = 7.1, 6.4 Hz, 1H), 7.20 (dd, J = 11.6, 7.9 |
| | | | | | | Hz, 2H), 6.76 (d, J = 2.6 Hz, |
| | | | | | | 1H), 1.59 (q, J = 4.4 Hz, |
| | | | | | | 2H), 1.15 (q, J = 4.6 Hz, |
| | | | | | | 2H). |
| 195 | 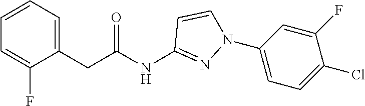 [SMILES: O=C(Cc1ccccc1F)Nc1ccn(-c2ccc(Cl)c(F)c2)n1] | B | 25 | >2 | 347.88 | 1H NMR (400 MHz, DMSO-d₆) δ 11.06 (s, 1H), 8.49 (d, J = 2.6 Hz, 1H), 7.85 (dd, J = 11.0, 2.3 Hz, 1H), 7.74-7.63 (m, 2H), 7.43-7.26 (m, 2H), 7.17 (dt, J = 9.2, 6.5 Hz, 2H), |
| | | | | | | 6.79 (d, J = 2.6 Hz, 1H), |
| | | | | | | 3.76 (s, 2H). |
| 196 | 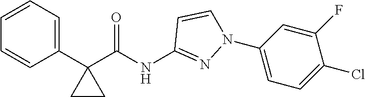 [SMILES: O=C(Nc1ccn(-c2ccc(Cl)c(F)c2)n1)C1(c2ccccc2)CC1] | B | 11 | >5 | 355.87 | 1H NMR (400 MHz, DMSO-d₆) δ 9.32 (s, 1H), 8.46 (d, J = 2.7 Hz, 1H), 7.82 (dd, J = 11.0, 2.4 Hz, 1H), 7.71-7.58 (m, 2H), 7.40 (ddd, J = 25.8, 17.8, 7.2 Hz, 5H), 6.80 (d, J = 2.6 |
| | | | | | | Hz, 1H), 1.48 (q, J = 4.3 Hz, |
| | | | | | | 2H), 1.14 (q, J = 4.3 Hz, |
| | | | | | | 2H). |
| 197 | 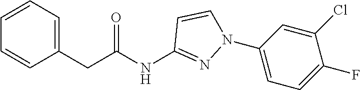 [SMILES: O=C(Cc1ccccc1)Nc1ccn(-c2ccc(F)c(Cl)c2)n1] | B | 19 | >5 | 329.91 | 1H NMR (400 MHz, DMSO-d₆) δ 10.99 (s, 1H), 8.45 (d, J = 2.6 Hz, 1H), 8.00 (dd, J = 6.4, 2.7 Hz, 1H), 7.80-7.75 (m, 1H), 7.56 (t, J = 9.0 Hz, 1H), |
| | | | | | | 7.32 (dd, J = 8.1, 5.6 Hz, |
| | | | | | | 4H), 7.24 (td, J = 5.5, 2.7 |
| | | | | | | Hz, 1H), 6.77 (d, J = 2.5 Hz, |
| | | | | | | 1H), 3.65 (s, 2H). |
| 198 | 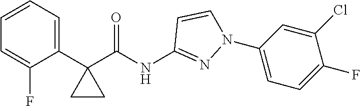 [SMILES: O=C(Nc1ccn(-c2ccc(F)c(Cl)c2)n1)C1(c2ccccc2F)CC1] | B | 4 | 0.72 | 373.91 | 1H NMR (400 MHz, DMSO-d₆) δ 9.38 (s, 1H), 8.40 (d, J = 2.7 Hz, 1H), 7.96 (dd, J = 6.4, 2.7 Hz, 1H), 7.73 (dt, J = 9.1, 3.6 Hz, 1H), 7.54-7.43 (m, 2H), 7.38 (dd, J = 8.0, 5.9 Hz, 1H), 7.20 (dd, J = 11.5, |
| | | | | | | 7.9 Hz, 2H), 6.73 (d, J = 2.6 |
| | | | | | | Hz, 1H), 1.59 (q, J = 4.4 Hz, |
| | | | | | | 2H), 1.15 (q, J = 4.5 Hz, |
| | | | | | | 2H). |
| 199 | 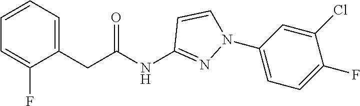 [SMILES: O=C(Cc1ccccc1F)Nc1ccn(-c2ccc(F)c(Cl)c2)n1] | B | 45 | >5 | 347.88 | 1H NMR (400 MHz, DMSO-d₆) δ 11.01 (s, 1H), 8.42 (d, J = 2.7 Hz, 1H), 7.99 (dd, J = 6.4, 2.7 Hz, 1H), 7.77 (dt, J = 9.0, 3.5 Hz, 1H), 7.55 (t, J = 9.0 Hz, 1H), 7.34 (ddd, J = 26.5, |
| | | | | | | 8.4, 6.4 Hz, 2H), 7.23- |
| | | | | | | 7.13 (m, 2H), 6.75 (d, J = |
| | | | | | | 2.6 Hz, 1H), 3.74 (s, 2H). |
| 200 | 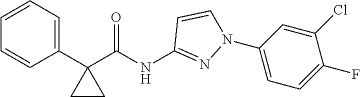 [SMILES: O=C(Nc1ccn(-c2ccc(F)c(Cl)c2)n1)C1(c2ccccc2)CC1] | B | 28 | 0.70 | 355.87 | 1H NMR (400 MHz, DMSO-d₆) δ 9.26 (s, 1H), 8.41 (d, J = 2.6 Hz, 1H), 7.95 (dd, J = 6.4, 2.7 Hz, 1H), 7.72 (dt, J = 9.0, 3.6 Hz, 1H), 7.50 (t, J = 9.0 Hz, 1H), 7.46-7.30 (m, 5H), |
| | | | | | | 6.76 (d, J = 2.6 Hz, 1H), |
| | | | | | | 1.47 (q, J = 4.2 Hz, 2H), |
| | | | | | | 1.12 (q, J = 4.3 Hz, 2H). |
| 201 | 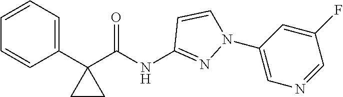 [SMILES: O=C(Nc1ccn(-c2cncc(F)c2)n1)C1(c2ccccc2)CC1] | J | 99 | 0.046 | 323.01 | 1H NMR (300 MHz, DMSO-d₆) δ 9.39 (s, 1H), 8.91 (t, J = 1.7 Hz, 1H), 8.53 (d, J = 2.7 Hz, 1H), 8.47 (d, J = 2.5 Hz, 1H), 8.09 (dt, J = 10.4, 2.4 Hz, 1H), 7.57-7.25 (m, 5H), |
| | | | | | | 6.86 (d, J = 2.7 Hz, 1H), |
| | | | | | | 1.49 (q, J = 4.1 Hz, 2H), |
| | | | | | | 1.14 (q, J = 4.2 Hz, 2H). |
| 202 | 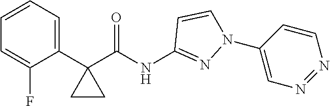 [SMILES: O=C(Nc1ccn(-c2ccnnc2)n1)C1(c2ccccc2F)CC1] | N | 16 | 0.095 | 324.09 | 1H NMR (400 MHz, DMSO-d₆) δ 9.81-9.69 (m, 2H), 9.21 (d, J = 5.9 Hz, 1H), 8.70 (d, J = 2.7 Hz, 1H), 7.92 (dd, J = 5.9, 2.8 Hz, 1H), 7.47 (t, J = 7.0 Hz, 1H), 7.44-7.35 (m, 1H), |
| | | | | | | 7.21 (dd, J = 11.7, 7.8 Hz, |
| | | | | | | 2H), 6.92 (d, J = 2.7 Hz, |
| | | | | | | 1H), 1.62 (q, J = 4.3 Hz, |
| | | | | | | 2H), 1.18 (q, J = 4.4 Hz, |
| | | | | | | 2H). |
| 203 | 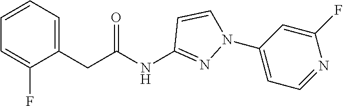 [SMILES: O=C(Cc1ccccc1F)Nc1ccn(-c2ccnc(F)c2)n1] | D | 89 | 0.10 | 315.00 | 1H NMR (400 MHz, DMSO-d₆) δ 11.18 (s, 1H), 8.65 (d, J = 2.8 Hz, 1H), 8.30 (d, J = 5.7 Hz, 1H), 7.74 (d, J = 5.7 Hz, 1H), 7.51 (d, J = 1.8 Hz, 1H), 7.35 (m, 2H), 7.23-7.14 |
| | | | | | | (m, 2H), 6.90 (d, J = 2.8 |
| | | | | | | Hz, 1H), 3.78 (s, 2H). |
| 204 | 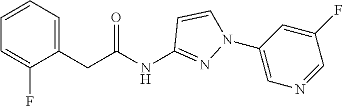 [SMILES: O=C(Cc1ccccc1F)Nc1ccn(-c2cncc(F)c2)n1] | J | 24 | 0.097 | 314.95 | 1H NMR (400 MHz, DMSO-d₆) δ 11.12 (s, 1H), 8.96 (d, J = 2.0 Hz, 1H), 8.56 (d, J = 2.7 Hz, 1H), 8.51 (d, J = 2.5 Hz, 1H), 8.19-8.05 (m, 1H), 7.38 (d, J = 7.7 Hz, 1H), 7.35- |
| | | | | | | 7.26 (m, 1H), 7.22-7.09 |
| | | | | | | (m, 2H), 6.85 (d, J = 2.6 |
| | | | | | | Hz, 1H), 3.77 (s, 2H). |
| 205 | 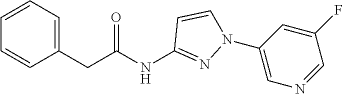 [SMILES: O=C(Cc1ccccc1)Nc1ccn(-c2cncc(F)c2)n1] | J | 54 | 0.72 | 297.04 | 1H NMR (400 MHz, DMSO-d₆) δ 11.08 (s, 1H), 8.95 (d, J = 2.1 Hz, 1H), 8.55 (d, J = 2.7 Hz, 1H), 8.50 (d, J = 2.5 Hz, 1H), 8.12 (dt, J = 10.4, 2.3 Hz, 1H), 7.37-7.29 (m, 4H), |
| | | | | | | 7.25 (dt, J = 9.2, 4.2 Hz, |
| | | | | | | 1H), 6.86 (d, J = 2.7 Hz, |
| | | | | | | 1H), 3.67 (s, 2H). |
| 206 | 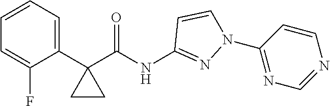 [SMILES: O=C(Nc1ccn(-c2ccncn2)n1)C1(c2ccccc2F)CC1] | O | 25 | 0.034 | 324.00 | 1H NMR (400 MHz, DMSO-d₆) δ 9.73 (s, 1H), 9.02 (d, J = 1.4 Hz, 1H), 8.82 (d, J = 5.8 Hz, 1H), 8.59 (d, J = 2.7 Hz, 1H), 7.70 (dd, J = 5.8, 1.4 Hz, 1H), 7.53-7.34 (m, 2H), |
| | | | | | | 7.24-7.19 (m, 2H), 6.89 |
| | | | | | | (d, J = 2.7 Hz, 1H), 1.63 (q, |
| | | | | | | J = 4.2 Hz, 2H), 1.18 (q, J = |
| | | | | | | 4.3 Hz, 2H). |
| 207 | 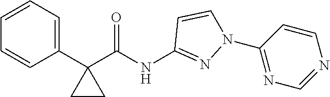 [SMILES: O=C(Nc1ccn(-c2ccncn2)n1)C1(c2ccccc2)CC1] | J | 16 | 0.036 | 306.04 | 1H NMR (400 MHz, DMSO-d₆) δ 9.63 (s, 1H), 9.02 (s, 1H), 8.81 (d, J = 5.6 Hz, 1H), 8.60 (d, J = 2.8 Hz, 1H), 7.72-7.65 (m, 1H), 7.45 (m, 2H), 7.42- 7.37 (m, 2H), 7.36-7.29 |
| | | | | | | (m, 1H), 6.92 (d, J = 2.8 |
| | | | | | | Hz, 1H), 1.50 (q, J = 4.3 Hz, |
| | | | | | | 2H), 1.15 (q, J = 4.3 Hz, |
| | | | | | | 2H). |
| 208 | 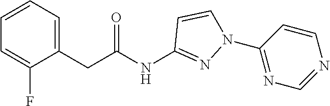 [SMILES: O=C(Cc1ccccc1F)Nc1ccn(-c2ccncn2)n1] | J | 58 | 2.3 | 297.99 | 1H NMR (400 MHz, DMSO-d₆) δ 11.23 (s, 1H), 9.04 (d, J = 1.3 Hz, 1H), 8.87 (dd, J = 5.7, 1.3 Hz, 1H), 8.62 (d, J = 2.6 Hz, 1H), 7.72 (dd, J = 5.7, 1.4 Hz, 1H), 7.54-7.26 (m, |
| | | | | | | 2H), 7.21-7.09 (m, 2H), |
| | | | | | | 6.92 (d, J = 2.7 Hz, 1H), |
| | | | | | | 3.79 (s, 2H). |
| 209 | 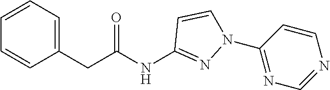 [SMILES: O=C(Cc1ccccc1)Nc1ccn(-c2ccncn2)n1] | J | 55 | 2.3 | 280.03 | 1H NMR (400 MHz, DMSO-d₆) δ 11.19 (s, 1H), 9.04 (d, J = 1.3 Hz, 1H), 8.36 (dd, J = 5.7, 1.4 Hz, 1H), 8.60 (d, J = 2.6 Hz, 1H), 7.71 (dd, J = 5.6, 1.3 Hz, 1H), 7.41-7.28 (m, |
| | | | | | | 4H), 7.26 (dt, J = 5.1, 2.3 |
| | | | | | | Hz, 1H), 6.92 (d, J = 2.8 Hz, |
| | | | | | | 1H), 3.69 (s, 2H). |
| 210 | 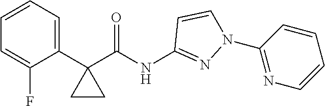 [SMILES: O=C(Nc1ccn(-c2ccccn2)n1)C1(c2ccccc2F)CC1] | B | 4 | 0.17 | 322.86 | 1H NMR (400 MHz, DMSO-d₆) δ 9.46 (s, 1H), 8.49 (d, J = 2.6 Hz, 1H), 8.43 (d, J = 4.9 Hz, 1H), 7.93 (t, J = 8.0 Hz, 1H), 7.72 (d, J = 8.2 Hz, 1H), 7.41 (q, J = 7.6 Hz, 1H), |
| | | | | | | 7.32 (p, J = 6.8, 6.1 Hz, |
| | | | | | | 2H), 7.22 (dd, J = 13.1, 6.1 |
| | | | | | | Hz, 2H), 6.78 (d, J = 2.7 Hz, |
| | | | | | | 1H), 1.61 (q, J = 4.4 Hz, |
| | | | | | | 2H), 1.16 (m, 2H). |
| 211 | 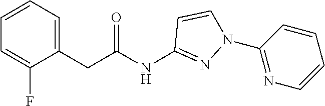 [SMILES: O=C(Cc1ccccc1F)Nc1ccn(-c2ccccn2)n1] | B | 10 | 1.7 | 296.87 | 1H NMR (400 MHz, DMSO-d₆) δ 11.09 (s, 1H), 8.52 (d, J = 2.6 Hz, 1H), 8.45 (dd, J = 4.9, 1.7 Hz, 1H), 7.99 (td, J = 7.8, 1.8 Hz, 1H), 7.76 (d, J = 8.2 Hz, 1H), 7.44-7.37 (m, 1H), |
| | | | | | | 7.33 (dd, J = 7.4, 4.6 Hz, |
| | | | | | | 2H), 7.24-7.12 (m, 2H), |
| | | | | | | 6.81 (d, J = 2.6 Hz, 1H), |
| | | | | | | 3.78 (s, 2H). |
| 212 | 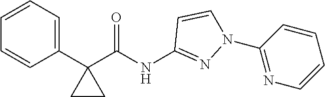 [SMILES: O=C(Nc1ccn(-c2ccccn2)n1)C1(c2ccccc2)CC1] | B | 6 | 0.19 | 304.90 | 1H NMR (400 MHz, DMSO-d₆) δ 9.31 (s, 1H), 8.48 (d, J = 2.7 Hz, 1H), 8.41 (d, J = 4.8 Hz, 1H), 7.91 (t, J = 8.1 Hz, 1H), 7.69 (d, J = 8.3 Hz, 1H), 7.45 (d, J = 7.5 Hz, 2H), |
| | | | | | | 7.39 (t, J = 7.4 Hz, 2H), |
| | | | | | | 7.36-7.26 (m, 2H), 6.80 |
| | | | | | | (d, J = 2.7 Hz, 1H), 1.48 (q, |
| | | | | | | J = 4.3 Hz, 2H), 1.13 (d, J = |
| | | | | | | 2.5 Hz, 2H). |
| 213 | 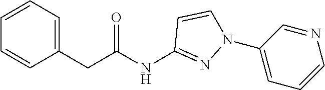 [SMILES: O=C(Cc1ccccc1)Nc1ccn(-c2cccnc2)n1] | B | 64 | 1.2 | 278.88 | 1H NMR (400 MHz, DMSO-d₆) δ 11.05 (s, 1H), 9.06 (d, J = 2.6 Hz, 1H), 8.51 (dd, J = 4.8, 1.7 Hz, 2H), 8.21 (ddd, J = 8.4, 2.6, 1.4 Hz, 1H), 7.59 (dd, J = 8.3, 4.8 Hz, 1H), 7.35- |
| | | | | | | 7.29 (m, 4H), 7.26-7.24 |
| | | | | | | (m, 1H), 6.83 (d, J = 2.6 |
| | | | | | | Hz, 1H), 3.67 (s, 2H). |
| 214 | 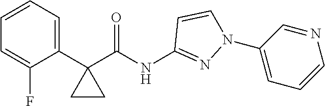 [SMILES: O=C(Nc1ccn(-c2cccnc2)n1)C1(c2ccccc2F)CC1] | B | 5 | 0.045 | 323.19 | 1H NMR (600 MHz, DMSO-d₆) δ 9.50 (s, 1H), 9.03 (d, J = 2.3 Hz, 1H), 8.49 (dd, J = 9.9, 3.4 Hz, 2H), 8.18-8.13 (m, 1H), 7.54 (dd, J = 8.3, 4.7 Hz, 1H), 7.48 (td, J = 7.8, 1.7 |
| | | | | | | Hz, 1H), 7.42-7.38 (m, |
| | | | | | | 1H), 7.23 7.18 (m, 2H), |
| | | | | | | 6.80 (d, J = 2.6 Hz, 1H), |
| | | | | | | 1.61 (q, J = 4.3 Hz, 2H), |
| | | | | | | 1.16 (q, J = 4.3 Hz, 2H). |
| 215 | 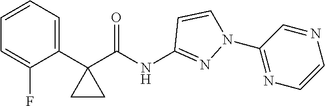 [SMILES: O=C(Nc1ccn(-c2cnccn2)n1)C1(c2ccccc2F)CC1] | B | 20 | 0.61 | 324.12 | 1H NMR (400 MHz, DMSO-d₆) δ 9.71 (s, 1H), 9.02 (d, J = 1.3 Hz, 1H), 8.55 (d, J = 2.6 Hz, 1H), 8.51 (t, J = 2.8 Hz, 2H), 7.50-7.45 (m, 1H), 7.43- 7.37 (m, 1H), 7.22 (dd, J = |
| | | | | | | 8.3, 6.1 Hz, 2H), 6.87 (d, |
| | | | | | | J = 2.7 Hz, 1H), 1.62 (q, J = |
| | | | | | | 4.3 Hz, 2H), 1.17 (dd, J = |
| | | | | | | 7.1, 4.4 Hz, 2H). |
| 216 | 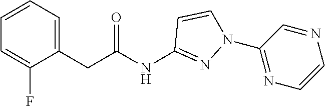 [SMILES: O=C(Cc1ccccc1F)Nc1ccn(-c2cnccn2)n1] | B | 6 | >5 | 298.03 | 1H NMR (400 MHz, DMSO-d₆) δ 11.22 (s, 1H), 9.05 (d, J = 1.0 Hz, 1H), 8.59 (d, J = 2.5 Hz, 1H), 8.54 (t, J = 3.0 Hz, 2H), 7.39 (dd, J = 8.4, 6.9 Hz, 1H), 7.36-7.29 (m, 1H), |
| | | | | | | 7.20-7.14 (m, 2H), 6.89 |
| | | | | | | (d, J = 2.7 Hz, 1H), 3.79 (s, |
| | | | | | | 2H). |
| 217 | 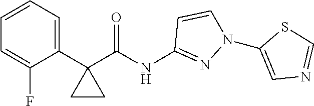 [SMILES: O=C(Nc1ccn(-c2cncs2)n1)C1(c2ccccc2F)CC1] | J | 18 | 0.084 | 328.96 | 1H NMR (400 MHz, DMSO-d₆) δ 9.61 (s, 1H), 8.82 (s, 1H), 8.32 (d, J = 2.4 Hz, 1H), 8.11 (s, 1H), 7.51-7.42 (m, 1H), 7.38 (s, 1H), 7.25-7.14 (m, 2H), 6.76 (d, J = 2.4 Hz, |
| | | | | | | 1H), 1.59 (s, 2H), 1.15 (s, |
| | | | | | | 2H). |
| 218 | 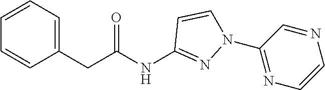 [SMILES: O=C(Cc1ccccc1)Nc1ccn(-c2cnccn2)n1] | B | 2 | >2 | 280.08 | 1H NMR (400 MHz, DMSO-d₆) δ 11.18 (s, 1H), 10.84 (s, 1H), 9.05 (s, 1H), 8.58 (d, J = 2.6 Hz, 1H), 8.53 (d, J = 2.5 Hz, 2H), 7.87 (d, J = 2.5 Hz, 1H), |
| | | | | | | 7.25 (m, 1H), 6.90 (m, 1H), |
| | | | | | | 6.56 (d, J = 2.4 Hz, 1H), |
| | | | | | | 5.69 (t, J = 6.3 Hz, 1H), |
| | | | | | | 3.69 (s, 2H). |
| 219 | 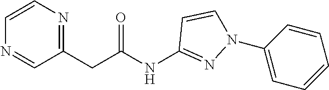 [SMILES: O=C(Cc1cnccn1)Nc1ccn(-c2ccccc2)n1] | D | 4 | >5 | 280.25 | 1H NMR (400 MHz, DMSO-d₆) δ 11.09 (s, 1H), 8.67 (s, 1H), 8.62-8.52 (m, 2H), 8.39 (dd, J = 8.4, 2.6 Hz, 2H), 7.75 (m, 2H), 7.48 (m, 2H), 7.27 (m, 1H), |
| | | | | | | 6.75 (d, J = 2.6 Hz, 1H), |
| | | | | | | 2.04 (s, 2H). |
| 220 | 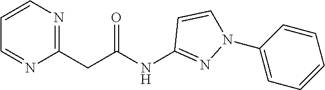 [SMILES: O=C(Cc1ncccn1)Nc1ccn(-c2ccccc2)n1] | D | 12 | >2 | 280.25 | 1H NMR (400 MHz, DMSO-d₆) δ 11.03 (s, 1H), 8.77 (d, J = 4.9 Hz, 2H), 8.40 (d, J = 2.6 Hz, 1H), 7.77 (d, J = 8.0 Hz, 2H), 7.49 (m, 2H), 7.41 (m, 1H), |
| | | | | | | 7.28 (m, 1H), 6.78 (d, J = |
| | | | | | | 2.6 Hz, 1H), 4.03 (s, 2H). |
| 221 | 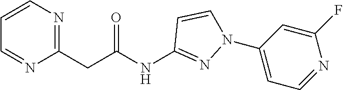 [SMILES: O=C(Cc1ncccn1)Nc1ccn(-c2ccnc(F)c2)n1] | D | 5 | >5 | 299.3 | 1H NMR (400 MHz, DMSO-d₆) δ 11.21 (s, 1H), 8.78 (d, J = 4.9 Hz, 2H), 8.66 (d, J = 2.8 Hz, 1H), 8.29 (m, 1H), 7.75 (m, 1H), 7.52 (m, 1H), 7.42 (t, J = |
| | | | | | | 4.9 Hz, 1H), 6.92 (d, J = 2.8 |
| | | | | | | Hz, 1H), 4.05 (s, 2H). |
| 222 | 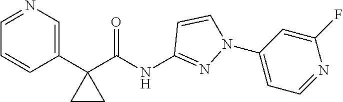 [SMILES: O=C(Nc1ccn(-c2ccnc(F)c2)n1)C1(c2cccnc2)CC1] | D | 26 | 2.0 | 324.22 | 1H NMR (400 MHz, DMSO-d₆) δ 10.22 (s, 1H), 8.84 (s, 1H), 8.72 (d, J = 5.3 Hz, 1H), 8.64 (d, J = 2.8 Hz, 1H), 8.26 (d, J = 5.3 Hz, 2H), 7.80-7.65 (m, 2H), 7.49 (s, 1H), 6.91 (d, J = |
| | | | | | | 2.8 Hz, 1H), 1.62 (m, 2H), |
| | | | | | | 1.30 (m, 2H). |
| 223 | 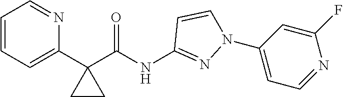 [SMILES: O=C(Nc1ccn(-c2ccnc(F)c2)n1)C1(c2ccccn2)CC1] | D | 26 | 0.070 | 324.22 | 1H NMR (400 MHz, DMSO-d₆) δ 11.65 (s, 1H), 8.65 (d, J = 2.8 Hz, 1H), 8.60-8.52 (m, 1H), 8.27 (m, 1H), 7.84-7.70 (m, 2H), 7.52 (m, 1H), 7.31 (m, 2H), 6.96 (d, J = 2.8 Hz, |
| | | | | | | 1H), 1.59 (m, 2H), 1.39 (m, |
| | | | | | | 2H). |
| 224 | 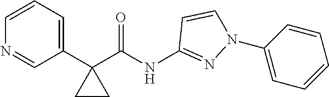 [SMILES: O=C(Nc1ccn(-c2ccccc2)n1)C1(c2cccnc2)CC1] | D | 66 | 1.4 | 305.30 | 1H NMR (400 MHz, DMSO-d₆) δ 9.87 (s, 1H), 8.61 (d, J = 2.3 Hz, 1H), 8.49 (d, J = 4.7 Hz, 1H), 8.36 (d, J = 2.6 Hz, 1H), 7.81 (m, 1H), 7.72 (m, 2H), 7.54-7.31 (m, 3H), 7.25 |
| | | | | | | (m, 1H), 6.74 (d, J = 2.6 |
| | | | | | | Hz, 1H), 1.53 (m, 2H), 1.18 |
| | | | | | | (m, 2H). |
| 225 | 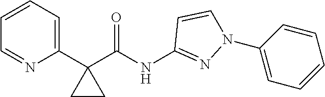 [SMILES: O=C(Nc1ccn(-c2ccccc2)n1)C1(c2ccccn2)CC1] | D | 41 | 0.075 | 305.30 | 1H NMR (400 MHz, DMSO-d₆) δ 11.42 (s, 1H), 8.57 (d, J = 4.9 Hz, 1H), 8.41 (d, J = 2.6 Hz, 1H), 7.78 (m, 3H), 7.48 (m, 2H), 7.30 (m, 3H), 6.82 (d, J = 2.6 Hz, 1H), 1.58 (m, 2H), |
| | | | | | | 1.37 (m, 2H). |
| 226 | 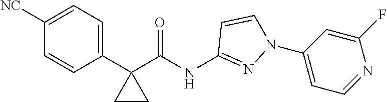 [SMILES: N#Cc1ccc(C2(C(=O)Nc3ccn(-c4ccnc(F)c4)n3)CC2)cc1] | D | 66 | 0.11 | 348.22 | 1H NMR (400 MHz, DMSO-d₆) δ 10.25 (s, 1H), 8.63 (d, J = 2.8 Hz, 1H), 8.26 (d, J = 5.7 Hz, 1H), 7.82 (d, J = 8.0 Hz, 2H), 7.71 (d, J = 5.7 Hz, 1H), 7.56 (d, J = 8.0 Hz, 2H), |
| | | | | | | 7.49 (s, 1H), 6.90 (d, J = |
| | | | | | | 2.8 Hz, 1H), 1.55 (m, 2H), |
| | | | | | | 1.22 (m, 2H). |
| 227 | 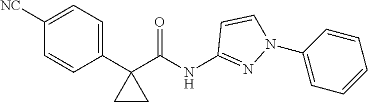 [SMILES: N#Cc1ccc(C2(C(=O)Nc3ccn(-c4ccccc4)n3)CC2)cc1] | D | 66 | 0.29 | 329.23 | 1H NMR (400 MHz, DMSO-d₆) δ 10.01 (s, 1H), 837 (d, J = 2.6 Hz, 1H), 7.82 (d, J = 8.1 Hz, 2H), 7.72 (d, J = 8.1 Hz, 2H), 7.57 (d, J = 8.0 Hz, 2H), 7.46 (m, 2H), 7.25 (m, 1H), |
| | | | | | | 6.75 (d, J = 2.6 Hz, 1H), |
| | | | | | | 1.54 (m, 2H), 1.19 (m, 2H). |
| 228 | 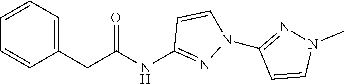 [SMILES: Cn1ccc(-n2ccc(NC(=O)Cc3ccccc3)n2)n1] | B | 3 | >5 | 282.11 | 1H NMR (400 MHz, DMSO-d₆) δ 10.95 (s, 1H), 8.04 (d, J = 2.5 Hz, 1H), 7.76 (d, J = 2.3 Hz, 1H), 7.38-7.27 (m, 4H), 7.24 (ddd, J = 8.6, 5.4, 2.7 Hz, 1H), 6.68 (d, J = 2.5 Hz, |
| | | | | | | 1H), 6.30 (d, J = 2.3 Hz, |
| | | | | | | 1H), 3.83 (s, 3H), 3.63 (s, |
| | | | | | | 2H). |
| 229 | 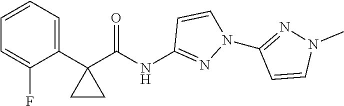 [SMILES: Cn1ccc(-n2ccc(NC(=O)C3(c4ccccc4F)CC3)n2)n1] | B | 1 | 3.5 | 326.14 | 1H NMR (400 MHz, DMSO-d₆) δ 9.30 (s, 1H), 8.04 (d, J = 2.4 Hz, 1H), 7.72 (d, J = 2.1 Hz, 1H), 7.52-7.44 (m, 1H), 7.43- 7.35 (m, 1H), 7.24-7.16 (m, 2H), 6.65 (d, J = 2.4 Hz, 1H), 6.28 (d, J = 2.3 Hz, 1H), 3.81 (s, 3H), 1.59 (q, |
| | | | | | | J = 4.2 Hz, 2H), 1.14 (dd, |
| | | | | | | J = 7.1, 4.3 Hz, 2H). |
| 230 | 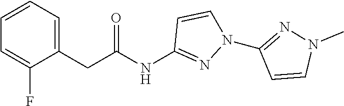 [SMILES: Cn1ccc(-n2ccc(NC(=O)Cc3ccccc3F)n2)n1] | B | 8 | >5 | 300.13 | 1H NMR (400 MHz, DMSO-d₆) δ 10.98 (s, 1H), 8.05 (d, J = 2.5 Hz, 1H), 7.77 (d, J = 2.2 Hz, 1H), 7.43-7.26 (m, 2H), 7.20- 7.08 (m, 2H), 6.67 (d, J = 2.5 Hz, 1H), 6.31 (d, J = 2.3 |
| | | | | | | Hz, 1H), 3.83 (s, 3H), 3.73 |
| | | | | | | (s, 2H). |
| 231 | 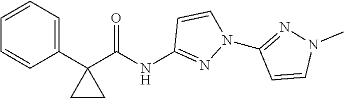 [SMILES: Cn1ccc(-n2ccc(NC(=O)C3(c4ccccc4)CC3)n2)n1] | B | 1 | >5 | 308.13 | 1H NMR (400 MHz, DMSO-d₆) δ 9.16 (s, 1H), 8.04 (d, J = 2.5 Hz, 1H), 7.72 (d, J = 2.1 Hz, 1H), 7.47-7.28 (m, 5H), 6.68 (d, J = 2.4 Hz, 1H), 6.28 (d, J = 2.3 Hz, 1H), 3.81 (s, |
| | | | | | | 3H), 1.47 (q, J = 4.1 Hz, |
| | | | | | | 2H), 1.11 (q, J = 4.2 Hz, |
| | | | | | | 2H). |
| 232 | 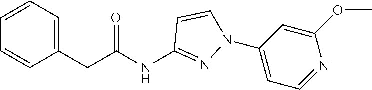 [SMILES: COc1cc(-n2ccc(NC(=O)Cc3ccccc3)n2)ccn1] | P | 48 | RND | 309.01 | 1H NMR (300 MHz, CDCl₃) δ 8.16 (dd, J = 5.8, 2.0 Hz, 1H), 7.97 (s, 1H), 7.87 (t, J = 2.4 Hz, 1H), 7.51-7.29 (m, 5H), 7.16-7.00 (m, 2H), 6.91 (d, J = 2.1 Hz, |
| | | | | | | 1H), 3.97 (d, J = 1.8 Hz, |
| | | | | | | 3H), 3.77 (s, 2H). |
| 233 | 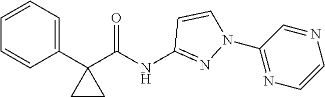 [SMILES: O=C(Nc1ccn(-c2cnccn2)n1)C1(c2ccccc2)CC1] | B | 72 | 1.5 | 306.04 | 1H NMR (400 MHz, DMSO-d₆) δ 9.59 (s, 1H), 9.00 (d, J = 1.4 Hz, 1H), 8.55 (d, J = 2.6 Hz, 1H), 8.53-8.46 (m, 2H), 7.50- 7.43 (m, 2H), 7.43-7.36 (m, 2H), 7.35-7.28 (m, |
| | | | | | | 1H), 6.89 (d, J = 2.7 Hz, |
| | | | | | | 1H), 1.49 (q, J = 4.2 Hz, |
| | | | | | | 2H), 1.14 (q, J = 4.3 Hz, |
| | | | | | | 2H). |
| 234 | 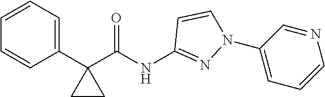 [SMILES: O=C(Nc1ccn(-c2cccnc2)n1)C1(c2ccccc2)CC1] | B | 39 | 0.029 | 305.01 | 1H NMR (400 MHz, DMSO-d₆) δ 9.38 (s, 1H), 9.00 (d, J = 2.6 Hz, 1H), 8.49 (d, J = 2.7 Hz, 1H), 8.45 (dd, J = 4.8, 1.4 Hz, 1H), 8.10 (ddd, J = 8.4, 2.8, 1.4 Hz, 1H), 7.53-7.28 (m, |
| | | | | | | 6H), 6.82 (d, J = 2.6 Hz, |
| | | | | | | 1H), 1.48 (q, J = 4.2 Hz, |
| | | | | | | 2H), 1.13 (q, J = 4.3 Hz, |
| | | | | | | 2H). |
| 235 | 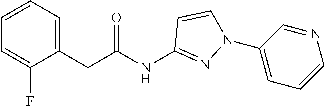 [SMILES: O=C(Cc1ccccc1F)Nc1ccn(-c2cccnc2)n1] | B | 70 | 0.32 | 296.95 | 1H NMR (400 MHz, DMSO-d₆) δ 11.08 (s, 1H), 9.05 (d, J = 2.6 Hz, 1H), 8.57-8.45 (m, 2H), 8.16 (m, 1H), 7.54 (m, 1H), 7.39 (td, J = 7.6, 1.7 Hz, 1H), 7.31 (m, 1H), 7.23-7.10 |
| | | | | | | (m, 2H), 6.81 (d, J = 2.6 |
| | | | | | | Hz, 1H), 3.76 (s, 2H). |
| 236 | 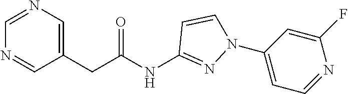 [SMILES: O=C(Cc1cncnc1)Nc1ccn(-c2ccnc(F)c2)n1] | D | 5 | >5 | 299.23 | 1H NMR (400 MHz, DMSO-d₆) δ 11.27 (s, 1H), 9.09 (s, 1H), 8.76 (s, 2H), 8.65 (d, J = 2.8 Hz, 1H), 8.30 (d, J = 5.7 Hz, 1H), 7.74 (d, J = 5.7 Hz, 1H), 7.51 (s, 1H), 7.25 (d, J = |
| | | | | | | 3.0 Hz, 1H), 7.12 (d, J = 3.1 |
| | | | | | | Hz, 1H), 7.00 (d, J = 3.0 Hz, |
| | | | | | | 1H), 6.90 (d, J = 2.8 Hz, |
| | | | | | | 1H), 3.82 (s, 2H). |
| 237 | 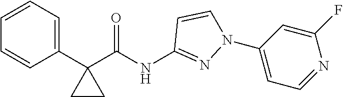 [SMILES: O=C(Nc1ccn(-c2ccnc(F)c2)n1)C1(c2ccccc2)CC1] | F | 23 | 0.022 | 232.01 | 1H NMR (300 MHz, DMSO-d₆) δ 9.47 (s, 1H), 8.63 (d, J = 2.6 Hz, 1H), 8.24 (d, J = 5.7 Hz, 1H), 7.81-7.61 (m, 1H), 7.53- 7.18 (m, 6H), 6.90 (d, J = 2.7 Hz, 1H), 1.49 (q, J = 4.1 |
| | | | | | | Hz, 2H), 1.14 (q, J = 4.2 Hz, |
| | | | | | | 2H). |
| 238 | 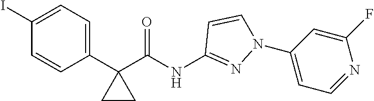 [SMILES: O=C(Nc1ccn(-c2ccnc(F)c2)n1)C1(c2ccc(I)cc2)CC1] | L | 41 | 0.12 | 448.96 | 1H NMR (300 MHz, CDCl₃) δ 8.29-8.11 (m, 1H), 7.89 (d, J = 2.3 Hz, 1H), 7.87- 7.75 (m, 2H), 7.64 (s, 1H), 7.45-7.33 (m, 1H), 7.28- 7.22 (m, 2H), 7.13 (d, J = 1.5 Hz, 1H), 7.07 (t, J = 2.4 |
| | | | | | | Hz, 1H), 1.85-1.66 (m, |
| | | | | | | 2H), 1.28-1.06 (m, 2H). |
| 239 | 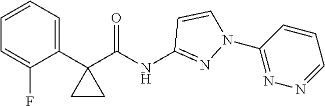 [SMILES: O=C(Nc1ccn(-c2cccnn2)n1)C1(c2ccccc2F)CC1] | F | 7 | 0.30 | 324.04 | 1H NMR (400 MHz, DMSO-d₆) δ 9.58 (s, 1H), 9.20-9.06 (m, 1H), 8.70 (d, J = 2.8 Hz, 1H), 7.99 (dd, J = 9.0, 1.5 Hz, 1H), 7.84 (dd, J = 8.9, 4.8 Hz, 1H), 7.51-7.44 (m, 1H), |
| | | | | | | 7.44-7.36 (m, 1H), 7.21 |
| | | | | | | (dd, J = 11.2, 7.9 Hz, 2H), |
| | | | | | | 6.87 (d, J = 2.7 Hz, 1H), |
| | | | | | | 1.61 (q, J = 4.3 Hz, 2H), |
| | | | | | | 1.17 (q, J = 4.5 Hz, 2H). |
| 240 | 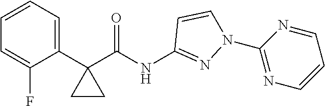 [SMILES: O=C(Nc1ccn(-c2ncccn2)n1)C1(c2ccccc2F)CC1] | J | 7 | 4.4 | 324.00 | 1H NMR (400 MHz, DMSO-d₆) δ 9.72 (s, 1H), 8.79 (d, J = 4.9 Hz, 2H), 8.54 (d, J = 2.8 Hz, 1H), 7.47 (m, 1H), 7.40 (m, 2H), 7.27-7.10 (m, 2H), 6.84 (d, J = 2.8 Hz, 1H), 1.62 (m, 2H), 1.16 (m, 2H). |
| 241 | 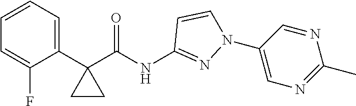 [SMILES: Cc1ncc(-n2ccc(NC(=O)C3(c4ccccc4F)CC3)n2)cn1] | K | 13 | >5 | 337.99 | 1H NMR (400 MHz, DMSO-d₆) δ 9.58 (s, 1H), 9.08 (s, 2H), 8.49 (d, J = 2.7 Hz, 1H), 7.51-7.44 (m, 1H), 7.39 (dd, J = 5.8, 3.6 Hz, 1H), 7.27-7.15 (m, 2H), 6.82 (d, J = 2.7 Hz, 1H), 2.64 (s, 3H), 1.60 (q, |
| | | | | | | J = 4.3 Hz, 2H), 1.16 (q, J = |
| | | | | | | 4.3 Hz, 2H). |
| 242 | 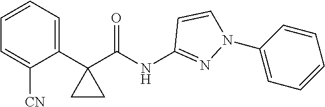 [SMILES: N#Cc1ccccc1C1(C(=O)Nc2ccn(-c3ccccc3)n2)CC1] | D | 10 | >2 | 329.23 | 1H NMR (400 MHz, DMSO-d₆) δ 9.61 (s, 1H), 8.39 (d, J = 2.6 Hz, 1H), 7.86 (dd, J = 7.8, 1.3 Hz, 1H), 7.79-7.71 (m, 2H), 7.69 (m, 1H), 7.63 (d, J = 7.7 Hz, 1H), 7.52 (m, 1H), 7.45 (m, 2H), 7.25 (m, 1H), |
| | | | | | | 6.72 (d, J = 2.6 Hz, 1H), |
| | | | | | | 1.76-1.70 (m, 2H), 1.27 |
| | | | | | | (m, 2H). |
| 243 | 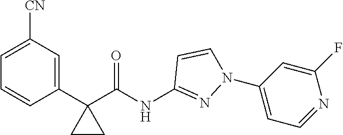 [SMILES: N#Cc1cccc(C2(C(=O)Nc3ccn(-c4ccnc(F)c4)n3)CC2)c1] | D | 57 | 0.31 | 348.20 | 1H NMR (400 MHz, DMSO-d₆) δ 10.11 (s, 1H), 8.65 (d, J = 2.8 Hz, 1H), 8.26 (d, J = 5.7 Hz, 1H), 7.86 (d, J = 1.7 Hz, 1H), 7.81-7.70 (m, 3H), 7.57 (m, 1H), 7.55-7.47 (m, 1H), 6.90 (s, 1H), 1.53 (m, 2H), 1.26-1.19 (m, 2H). |
| 244 | 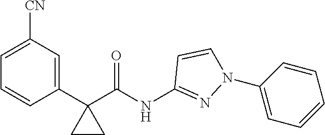 [SMILES: N#Cc1cccc(C2(C(=O)Nc3ccn(-c4ccccc4)n3)CC2)c1] | D | 28 | 0.28 | 329.26 | 1H NMR (400 MHz, DMSO-d₆) δ 9.91 (s, 1H), 8.39 (d, J = 2.6 Hz, 1H), 7.87 (d, J = 1.7 Hz, 1H), 7.80-7.70 (m, 3H), 7.57 (m, 1H), 7.45 (m, 2H), 7.25 (m, 1H), 6.75 (d, J = 2.6 Hz, 1H), 1.52 (m, 2H), 1.19 (m, 2H). |
| 245 | 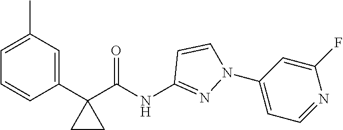 [SMILES: Cc1cccc(C2(C(=O)Nc3ccn(-c4ccnc(F)c4)n3)CC2)c1] | D | 33 | 0.26 | 337.26 | 1H NMR (400 MHz, DMSO-d₆) δ 9.42 (s, 1H), 8.64 (d, J = 2.7 Hz, 1H), 8.25 (d, J = 5.7 Hz, 1H), 7.71 (dt, J = 5.8, 1.5 Hz, 1H), 7.49 (d, J = 1.8 Hz, 1H), 7.33-7.20 (m, 3H), 7.14 (d, J = 7.2 Hz, 1H), 6.90 (d, J = 2.7 Hz, 1H), |
| | | | | | | 2.32 (s, 3H), 1.47 (m, 2H), |
| | | | | | | 1.13 (m, 2H). |
| 246 | 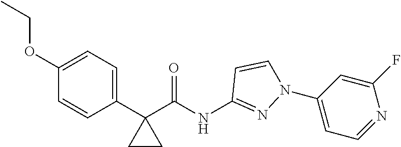 [SMILES: CCOc1ccc(C2(C(=O)Nc3ccn(-c4ccnc(F)c4)n3)CC2)cc1] | D | 6 | 2.8 | 367.22 | 1H NMR (400 MHz, DMSO-d₆) δ 9.21 (s, 1H), 8.64 (d, J = 2.8 Hz, 1H), 8.24 (d, J = 5.7 Hz, 1H), 7.70 (d, J = 5.5 Hz, 1H), 7.49 (d, J = 1.7 Hz, 1H), 7.45-7.32 (m, 2H), 6.99- 6.87 (m, 3H), 4.04 (q, J = 7.0 Hz, 2H), 1.45 (m, 2H), 1.34 (t, J = 7.0 Hz, 3H), |
| | | | | | | 1.09 (m, 2H). |
| 247 | 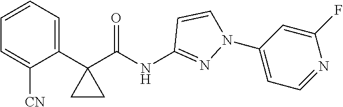 [SMILES: N#Cc1ccccc1C1(C(=O)Nc2ccn(-c3ccnc(F)c3)n2)CC1] | D | 20 | 2.5 | 348.20 | 1H NMR (400 MHz, DMSO-d₆) δ 9.87 (s, 1H), 8.64 (d, J = 2.8 Hz, 1H), 8.26 (d, J = 5.7 Hz, 1H), 7.86 (m, 1H), 7.74-7.65 (m, 2H), 7.63 (m, 1H), 7.58- 7.48 (m, 2H), 6.88 (m, 1H), 1.76 (m, 2H), 1.30 (m, |
| | | | | | | 2H). |
| 248 | 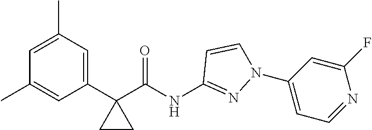 [SMILES: Cc1cc(C)cc(C2(C(=O)Nc3ccn(-c4ccnc(F)c4)n3)CC2)c1] | D | 27 | >5 | 351.48 | 1H NMR (400 MHz, DMSO-d₆) δ 9.31 (s, 1H), 8.64 (d, J = 2.8 Hz, 1H), 8.25 (m, 1H), 7.70 (m, 1H), 7.49 (d, J = 1.7 Hz, 1H), 7.06 (s, 2H), 6.96 (s, 1H), 6.90 (d, J = 2.7 Hz, 1H), 2.28 (s, 6H), 1.45 (m, 2H), 1.11 (m, 2H). |
| 249 | 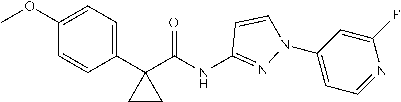 [SMILES: COc1ccc(C2(C(=O)Nc3ccn(-c4ccnc(F)c4)n3)CC2)cc1] | D | 31 | 0.64 | 353.21 | 1H NMR (400 MHz, DMSO-d₆) δ 9.23 (s, 1H), 8.64 (d, J = 2.8 Hz, 1H), 8.24 (m, 1H), 7.71 (m, 1H), 7.49 (d, J = 1.8 Hz, 1H), 7.44-7.35 (m, 2H), 7.00- 6.93 (m, 2H), 6.90 (d, J = 2.7 Hz, 1H), 3.77 (s, 3H), |
| | | | | | | 1.46 (m, 2H), 1.09 (m, 2H). |
| 250 | 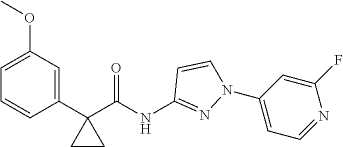 [SMILES: COc1cccc(C2(C(=O)Nc3ccn(-c4ccnc(F)c4)n3)CC2)c1] | D | 44 | 0.17 | 353.21 | 1H NMR (400 MHz, DMSO-d₆) δ 9.48 (s, 1H), 8.64 (d, J = 2.8 Hz, 1H), 8.25 (d, J = 5.7 Hz, 1H), 7.71 (m, 1H), 7.49 (m, 1H), 7.31 (t, J = 7.9 Hz, 1H), 7.07-6.97 (m, 2H), 6.90 (m, 2H), 3.78 (s, 3H), 1.47 (q, J = 4.2 Hz, 2H), 1.15 (q, J = 4.3 Hz, 2H). |
| 251 | 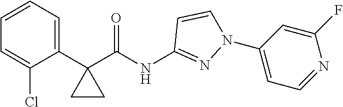 [SMILES: O=C(Nc1ccn(-c2ccnc(F)c2)n1)C1(c2ccccc2Cl)CC1] | D | 7 | 0.11 | 357.17 | 1H NMR (400 MHz, DMSO-d₆) δ 9.43 (s, 1H), 8.63 (d, J = 2.8 Hz, 1H), 8.25 (m, 1H), 7.71 (m, 1H), 7.58-7.44 (m, 3H), 7.44- 7.31 (m, 2H), 6.88 (d, J = 2.8 Hz, 1H), 1.68 (m, 2H), 1.18 (m, 2H). |
| 252 | 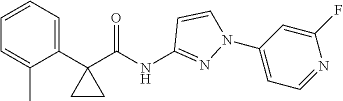 [SMILES: Cc1ccccc1C1(C(=O)Nc2ccn(-c3ccnc(F)c3)n2)CC1] | D | 8 | 0.40 | 337.22 | 1H NMR (400 MHz, DMSO-d₆) δ 8.91 (s, 1H), 8.64 (d, J = 2.8 Hz, 1H), 8.24 (d, J = 5.7 Hz, 1H), 7.70 (d, J = 5.8 Hz, 1H), 7.49 (d, J = 1.8 Hz, 1H), 7.43 (d, J = 6.9 Hz, 1H), 7.32-7.23 (m, 3H), 6.89 |
| | | | | | | (d, J = 2.8 Hz, 1H), 2.31 (s, |
| | | | | | | 3H), 1.62 (m, 2H), 1.14 (m, |
| | | | | | | 2H). |
| 253 | 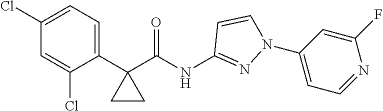 [SMILES: O=C(Nc1ccn(-c2ccnc(F)c2)n1)C1(c2ccc(Cl)cc2Cl)CC1] | D | 23 | 0.12 | 391.16 | 1H NMR (400 MHz, DMSO-d₆) δ 9.80 (s, 1H), 8.63 (d, J = 2.8 Hz, 1H), 8.25 (m, 1H), 7.80-7.69 (m, 1H), 7.65 (m, 1H), 7.59- 7.49 (m, 2H), 7.43 (dd, J = 8.3, 2.2 Hz, 1H), 6.88 (d, J = 2.8 Hz, 1H), 1.67 (m, |
| | | | | | | 2H), 1.17 (m, 2H). |
| 254 | 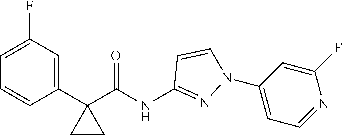 [SMILES: O=C(Nc1ccn(-c2ccnc(F)c2)n1)C1(c2cccc(F)c2)CC1] | D | 35 | 0.19 | 341.25 | 1H NMR (400 MHz, DMSO-d₆) δ 9.95 (s, 1H), 8.64 (d, J = 2.8 Hz, 1H), 8.26 (d, J = 5.7 Hz, 1H), 7.72 (dd, J = 5.7, 1.7 Hz, 1H), 7.50 (d, J = 1.7 Hz, 1H), 7.41 (m, 1H), 7.28- 7.19 (m, 2H), 7.14 (m, 1H), 6.90 (d, J = 2.8 Hz, 1H), |
| | | | | | | 1.49 (m, 2H), 1.17 (m, 2H). |
| 255 | 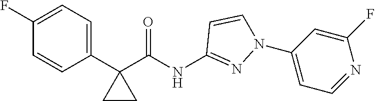 [SMILES: O=C(Nc1ccn(-c2ccnc(F)c2)n1)C1(c2ccc(F)cc2)CC1] | D | 35 | 0.10 | 341.22 | 1H NMR (400 MHz, DMSO-d₆) δ 9.72 (s, 1H), 8.64 (d, J = 2.8 Hz, 1H), 8.26 (d, J = 5.7 Hz, 1H), 7.71 (m, 1H), 7.55-7.44 (m, 3H), 7.26-7.14 (m, 2H), 6.89 (d, J = 2.8 Hz, |
| | | | | | | 1H), 1.49 (m, 2H), 1.12 (m, |
| | | | | | | 2H). |
| 256 | 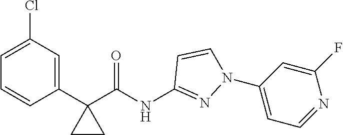 [SMILES: O=C(Nc1ccn(-c2ccnc(F)c2)n1)C1(c2cccc(Cl)c2)CC1] | D | 45 | 0.30 | 357.17 | 1H NMR (400 MHz, DMSO-d₆) δ 10.04 (s, 1H), 8.65 (6, J = 2.8 Hz, 1H), 8.27 (d, J = 5.7 Hz, 1H), 7.73 (m, 1H), 7.53-7.44 (m, 2H), 7.44-7.33 (m, 3H), 6.90 (d, J = 2.8 Hz, 1H), 1.50 (m, 2H), 1.18 (m, 2H). |
| 257 | 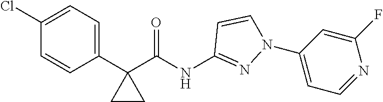 [SMILES: O=C(Nc1ccn(-c2ccnc(F)c2)n1)C1(c2ccc(Cl)cc2)CC1] | D | 48 | 0.34 | 357.21 | 1H NMR (300 MHz, CDCl₃) δ 8.18 (d, J = 5.7 Hz, 1H), 7.87 (d, J = 2.8 Hz, 1H), 7.62 (s, 1H), 7.44 (s, 4H), 7.33 (ddd, J = 5.7, 1.9, 1.2 Hz, 1H), 7.11 (t, J = 1.5 Hz, 1H), 7.04 (d, J = 2.8 Hz, |
| | | | | | | 1H), 1.79-1.70 (m, 2H), |
| | | | | | | 1.23-1.15 (m, 2H). |
| 258 | 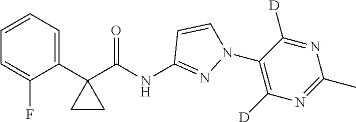 [SMILES: [2H]c1nc(C)nc([2H])c1-n1ccc(NC(=O)C2(c3ccccc3F)CC2)n1] | K | 32 | >5 | 340.11 | 1H NMR (400 MHz, DMSO-d₆) δ 9.59 (s, 1H), 8.50 (d, J = 2.6 Hz, 1H), 7.47 (t, J = 7.7 Hz, 1H), 7.43-7.35 (m, 1H), 7.26- 7.16 (m, 2H), 6.82 (d, J = 2.6 Hz, 1H), 2.63 (s, 2H), 1.60 (q, J = 4.3 Hz, 2H), 1.16 (q, J = 4.2 Hz, 2H). |
| 259 | 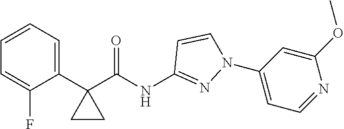 [SMILES: COc1cc(-n2ccc(NC(=O)C3(c4ccccc4F)CC3)n2)ccn1] | K | 24 | 0.051 | 353.11 | 1H NMR (400 MHz, DMSO-d₆) δ 9.60 (s, 1H), 8.56 (d, J = 2.7 Hz, 1H), 8.17 (d, J = 5.8 Hz, 1H), 7.47 (t, J = 7.6 Hz, 1H), 7.39 (dd, J = 5.7, 1.8 Hz, 2H), 7.20 (m, 2H), 7.14 (d, J = 1.7 Hz, 1H), 6.81 (d, J = 2.7 Hz, 1H), 3.87 (s, 3H), |
| | | | | | | 1.61 (q, J = 4.3 Hz, 2H), |
| | | | | | | 1.16 (q, J = 4.3 Hz, 2H). |
| 260 | 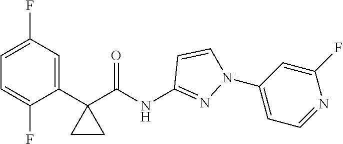 [SMILES: O=C(Nc1ccn(-c2ccnc(F)c2)n1)C1(c2cc(F)ccc2F)CC1] | M | 38 | 0.17 | 359.37 | 1H NMR (400 MHz, DMSO-d₆) δ 9.98 (s, 1H), 8.63 (d, J = 2.7 Hz, 1H), 8.26 (m, 1H), 7.72 (m, 1H), 7.50 (m, 1H), 7.32 (m, 1H), 7.24 (m, 2H), 6.88 (d, J = 2.7 Hz, 1H), 1.59 (m, 2H), 1.21 (m, 2H). |
| 261 | 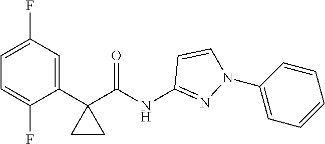 [SMILES: O=C(Nc1ccn(-c2ccccc2)n1)C1(c2cc(F)ccc2F)CC1] | M | 48 | 0.16 | 340.37 | 1H NMR (400 MHz, DMSO-d₆) δ 9.73 (s, 1H), 8.37 (d, J = 2.6 Hz, 1H), 7.74 (m, 2H), 7.45 (m, 2H), 7.36-7.18 (m, 4H), 6.73 (d, J = 2.6 Hz, 1H), 1.58 (m, 2H), 1.18 (m, 2H). |
| 262 | 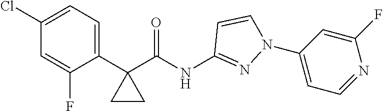 [SMILES: O=C(Nc1ccn(-c2ccnc(F)c2)n1)C1(c2ccc(Cl)cc2F)CC1] | M | 40 | 0.30 | 375.33 | 1H NMR (400 MHz, DMSO-d₆) δ 9.95 (s, 1H), 8.63 (d, J = 2.8 Hz, 1H), 8.26 (m, 1H), 7.72 (m, 1H), 7.52-7.41 (m, 3H), 7.29 (m, 1H), 6.87 (d, J = 2.8 Hz, 1H), 1.61 (m, 2H), 1.17 (m, 2H). |
| 263 | 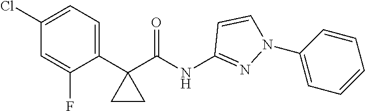 [SMILES: O=C(Nc1ccn(-c2ccccc2)n1)C1(c2ccc(Cl)cc2F)CC1] | M | 50 | >5 | 356.33 | 1H NMR (400 MHz, DMSO-d₆) δ 9.71 (s, 1H), 8.37 (d, J = 2.6 Hz, 1H), 7.74 (m, 2H), 7.45 (m, 4H), 7.31-7.21 (m, 2H), 6.72 (d, J = 2.6 Hz, 1H), 1.59 (m, 2H), 1.14 (m, 2H). |
| 264 | 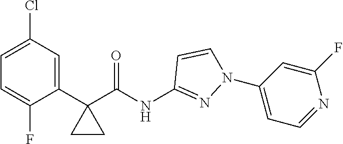 [SMILES: O=C(Nc1ccn(-c2ccnc(F)c2)n1)C1(c2cc(Cl)ccc2F)CC1] | M | 9 | 0.19 | 375.33 | 1H NMR (400 MHz, DMSO-d₆) δ 10.05 (s, 1H), 8.63 (d, J = 2.8 Hz, 1H), 8.26 (m, 1H), 7.72 (m, 1H), 7.53-7.40 (m, 3H), 7.25 (m, 1H), 6.88 (d, J = 2.8 Hz, 1H), 1.59 (m, 2H), 1.21 (m, 2H). |
| 265 | 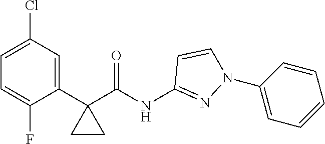 [SMILES: O=C(Nc1ccn(-c2ccccc2)n1)C1(c2cc(Cl)ccc2F)CC1] | M | 10 | 0.24 | 356.40 | 1H NMR (400 MHz, DMSO-d₆) δ 9.82 (s, 1H), 8.37 (d, J = 2.5 Hz, 1H), 7.74 (m, 2H), 7.53-7.39 (m, 4H), 7.31-7.21 (m, 2H), 6.72 (d, J = 2.5 Hz, 1H), 1.58 (m, 2H), 1.19 (m, 2H). |
| 266 | 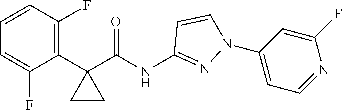 [SMILES: O=C(Nc1ccn(-c2ccnc(F)c2)n1)C1(c2c(F)cccc2F)CC1] | M | 20 | 0.017 | 359.37 | 1H NMR (400 MHz, DMSO-d₆) δ 10.04 (s, 1H), 8.63 (d, J = 2.7 Hz, 1H), 8.26 (m, 1H), 7.73 (m, 1H), 7.53-7.39 (m, 2H), 7.10 (m, 2H), 6.87 (d, J = 2.7 Hz, 1H), 1.75 (m, 2H), 1.20 (m, 2H). |
| 267 | 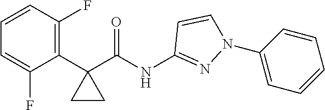 [SMILES: O=C(Nc1ccn(-c2ccccc2)n1)C1(c2c(F)cccc2F)CC1] | M | 20 | 0.013 | 340.37 | 1H NMR (400 MHz, DMSO-d₆) δ 9.80 (s, 1H), 8.37 (d, J = 2.6 Hz, 1H), 7.74 (m, 2H), 7.48-7.42 (m, 3H), 7.25 (m, 1H), 7.10 (m, 2H), 6.72 (d, J = 2.6 Hz, 1H), 1.73 (m, 2H), 1.18 (m, 2H). |
| 268 | 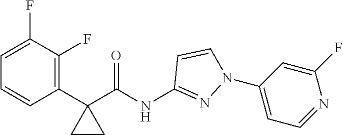 [SMILES: O=C(Nc1ccn(-c2ccnc(F)c2)n1)C1(c2cccc(F)c2F)CC1] | M | 30 | 0.14 | 359.37 | 1H NMR (400 MHz, DMSO-d₆) δ 9.95 (s, 1H), 8.63 (d, J = 2.7 Hz, 1H), 8.26 (d, J = 5.7 Hz, 1H), 7.73 (d, J = 5.7 Hz, 1H), 7.50 (m, 1H), 7.46-7.33 (m, 1H), 7.32-7.16 (m, 2H), 6.88 (d, J = 2.7 Hz, 1H), 1.64 (m, 2H), 1.22 (m, 2H). |
| 269 | 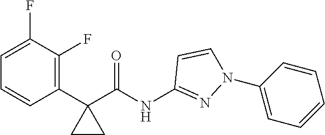 [SMILES: O=C(Nc1ccn(-c2ccccc2)n1)C1(c2cccc(F)c2F)CC1] | M | 30 | 0.098 | 340.37 | 1H NMR (400 MHz, DMSO-d₆) δ 9.70 (s, 1H), 8.37 (d, J = 2.6 Hz, 1H), 7.74 (m, 2H), 7.42 (m, 3H), 7.32-7.18 (m, 3H), 6.72 (d, J = 2.6 Hz, 1H), 1.63 (m, 2H), 1.19 (m, 2H). |
| 270 | 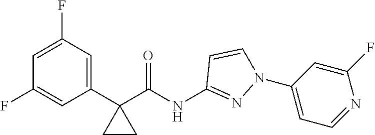 [SMILES: O=C(Nc1ccn(-c2ccnc(F)c2)n1)C1(c2cc(F)cc(F)c2)CC1] | M | 30 | 0.28 | 359.37 | 1H NMR (400 MHz, DMSO-d₆) δ 10.14 (s, 1H), 8.64 (d, J = 2.7 Hz, 1H), 8.27 (01, 1H), 7.73 (m, 1H), 7.50 (m, 1H), 7.22-7.07 (m, 3H), 6.90 (d, J = 2.7 Hz, 1H), 1.51 (m, 2H), 1.21 (m, 2H). |
| 271 | 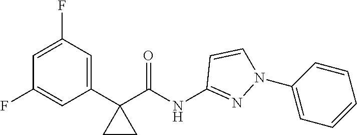 [SMILES: O=C(Nc1ccn(-c2ccccc2)n1)C1(c2cc(F)cc(F)c2)CC1] | M | 40 | 0.65 | 340.37 | 1H NMR (400 MHz, DMSO-d₆) δ 9.92 (s, 1H), 8.39 (d, J = 2.6 Hz, 1H), 7.74 (m, 2H), 7.46 (m, 2H), 7.31-7.07 (m, 4H), 6.75 (d, J = 2.6 Hz, 1H), 1.49 (m, 2H), 1.19 (m, 2H). |
| 272 | 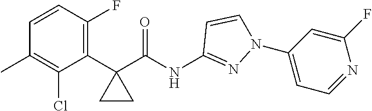 [SMILES: Cc1ccc(F)c(C2(C(=O)Nc3ccn(-c4ccnc(F)c4)n3)CC2)c1Cl] | M | 9 | 0.46 | 389.38 | 1H NMR (400 MHz, DMSO-d₆) δ 9.87 (s, 1H), 8.62 (d, J = 2.7 Hz, 1H), 8.26 (m, 1H), 7.72 (m, 1H), 7.50 (m, 1H), 7.40 (m, 1H), 7.15 (m, 1H), 6.88 (d, J = 2.7 Hz, 1H), 1.81 (m, 2H), 1.19 (m, 2H). |
| 273 | 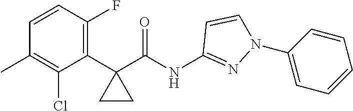 [SMILES: Cc1ccc(F)c(C2(C(=O)Nc3ccn(-c4ccccc4)n3)CC2)c1Cl] | M | 10 | RND | 370.38 | 1H NMR (400 MHz, DMSO-d₆) δ 9.60 (s, 1H), 8.37 (d, J = 2.6 Hz, 1H), 7.74 (m, 2H), 7.42 (m, 3H), 7.25 (m, 1H), 7.15 (m, 1H), 6.88 (s, 1H), 6.72 (d, J = 2.6 Hz, 1H), 1.80 (m, 2H), 1.17 (m, 2H). |
| 274 | 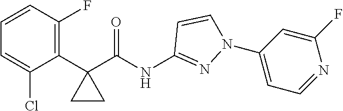 [SMILES: O=C(Nc1ccn(-c2ccnc(F)c2)n1)C1(c2c(F)cccc2Cl)CC1] | M | 30 | 0.037 | 375.33 | 1H NMR (400 MHz, DMSO-d₆) δ 9.95 (s, 1H), 8.62 (d, J = 2.7 Hz, 1H), 8.26 (m, 1H), 7.73 (m, 1H), 7.53-7.33 (m, 3H), 7.24 (m, 1H), 6.88 (d, J = 2.7 Hz, 1H), 1.82 (m, 2H), 1.22 (m, 2H). |
| 275 | 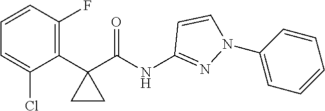 [SMILES: O=C(Nc1ccn(-c2ccccc2)n1)C1(c2c(F)cccc2Cl)CC1] | M | 40 | 0.050 | 356.33 | 1H NMR (400 MHz, DMSO-d₆) δ .70 (s, 1H), 8.37 (d, J = 2.6 Hz, 1H), 7.74 (m, 2H), 7.48-7.32 (m, 3H), 7.28-7.18 (m, 2H), 6.73 (d, J = 2.6 Hz, 1H), 1.80 (m, 2H), 1.22- 1.14 (m, 2H). |
| 276 | 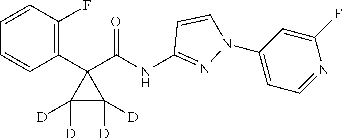 [SMILES: [2H]C1([2H])C([2H])([2H])C1(C(=O)Nc1ccn(-c2ccnc(F)c2)n1)c1ccccc1F] | N | 77 | 0.011 | 345.00 | 1H NMR (300 MHz, CDCl₃) δ 8.17 (d, J = 5.7 Hz, 1H), 7.87 (dd, J = 2.8, 0.5 Hz, 1H), 7.69 (s, 1H), 7.50- 7.38 (m, 2H), 7.32 (ddd, J = 5.7, 1.9, 1.3 Hz, 1H), 7.25 (d, J = 5.4 Hz, 1H), 7.22-7.13 (m, 1H), 7.10 (t, J = 1.7 Hz, 1H), 7.07 (d, J = 2.7 Hz, 1H). |
| 277 | 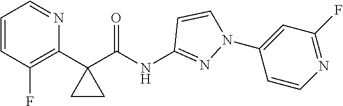 [SMILES: O=C(Nc1ccn(-c2ccnc(F)c2)n1)C1(c2ncccc2F)CC1] | D | 33 | 0.12 | 342.17 | 1H NMR (400 MHz, DMSO-d₆) δ 10.39 (s, 1H), 8.65 (d, J = 2.8 Hz, 1H), 8.38 (m, 1H), 8.27 (d, J = 5.7 Hz, 1H), 7.76-7.60 (m, 2H), 7.50 (d, J = 1.7 Hz, 1H), 7.44 (m, 1H), 6.91 (d, J = 2.8 Hz, 1H), 1.63 (m, 2H), 1.41 (m, 2H). |
| 278 | 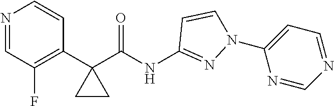 [SMILES: O=C(Nc1ccn(-c2ccncn2)n1)C1(c2ccncc2F)CC1] | D | 27 | >5 | 325.20 | 1H NMR (400 MHz, DMSO-d₆) δ 10.30 (s, 1H), 9.03 (d, J = 1.2 Hz, 1H), 8.83 (d, J = 5.6 Hz, 1H), 8.61 (d, J = 2.8 Hz, 1H), 8.54 (m, 1H), 8.42 (m, 1H), 7.69 (m, 1H), 7.56-7.42 |
| | | | | | | (m, 1H), 6.90 (m, 1H), 1.67 |
| | | | | | | (m, 2H), 1.28 (m, 2H). |
| 279 | 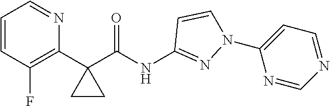 [SMILES: O=C(Nc1ccn(-c2ccncn2)n1)C1(c2ncccc2F)CC1] | D | 18 | 0.52 | 325.20 | 1H NMR (400 MHz, DMSO-d₆) δ 10.47 (s, 1H), 9.03 (d, J = 1.2 Hz, 1H), 8.83 (d, J = 5.6 Hz, 1H), 8.61 (d, J = 2.8 Hz, 1H), 8.38 (m, 1H), 7.73-7.63 (m, 2H), 7.44 (m, 1H), 6.92 |
| | | | | | | (d, J = 2.8 Hz, 1H), 1.64 |
| | | | | | | (m, 2H), 1.41 (m, 2H). |
| 280 | 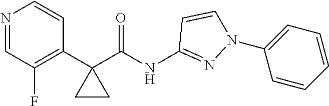 [SMILES: O=C(Nc1ccn(-c2ccccc2)n1)C1(c2ccncc2F)CC1] | D | 26 | 0.77 | 323.18 | 1H NMR (400 MHz, DMSO-d₆) δ 10.02 (s, 1H), 8.53 (6, J = 1.8 Hz, 1H), 8.40 (m, 2H), 7.75 (m, 2H), 7.50-7.40 (m, 3H), 7.31- 7.21 (m, 1H), 6.73 (6, J = 2.5 Hz, 1H), 1.65 (m, 2H), |
| | | | | | | 1.24 (m, 2H). |
| 281 | 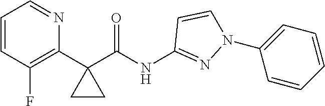 [SMILES: O=C(Nc1ccn(-c2ccccc2)n1)C1(c2ncccc2F)CC1] | D | 16 | 0.058 | 323.18 | 1H NMR (400 MHz, DMSO-d₆) δ 10.16 (s, 1H), 8.38 (m, 2H), 7.77-7.62 (m, 3H), 7.49-7.39 (m, 3H), 7.29-7.20 (m, 1H), 6.75 (d, J = 2.6 Hz, 1H), 1.63 (m, 2H), 1.38 (m, 2H). |
| 282 | 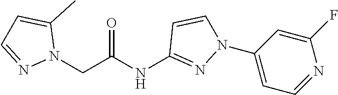 [SMILES: Cc1ccnn1CC(=O)Nc1ccn(-c2ccnc(F)c2)n1] | D | 56 | >2 | 301.27 | 1H NMR (400 MHz, DMSO-d₆) δ 11.28 (s, 1H), 8.68 (d, J = 2.8 Hz, 1H), 8.31 (d, J = 5.7 Hz, 1H), 7.75 (d, J = 5.7 Hz, 1H), 7.53 (m, 1H), 7.31 (d, J = 1.6 Hz, 1H), 6.89 (d, J = 2.8 |
| | | | | | | Hz, 1H), 6.05 (d, J = 1.6 Hz, |
| | | | | | | 1H), 5.01 (s, 2H), 2.25 (s, |
| | | | | | | 3H). |
| 283 | 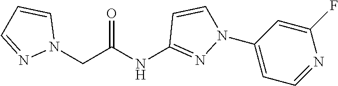 [SMILES: O=C(Cn1cccn1)Nc1ccn(-c2ccnc(F)c2)n1] | D | 61 | >5 | 287.30 | 1H NMR (400 MHz, DMSO-d₆) δ 8.68 (6, J = 2.8 Hz, 1H), 8.30 (d, J = 5.7 Hz, 1H), 7.78-7.74 (m, 2H), 7.53 (d, J = 1.8 Hz, 1H), 7.47 (d, J = 1.8 Hz, |
| | | | | | | 1H), 6.89 (d, J = 2.8 Hz, |
| | | | | | | 1H), 6.28 (m, 1H), 5.07 (s, |
| | | | | | | 2H). |
| 284 | 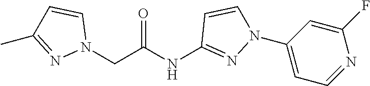 [SMILES: Cc1ccn(CC(=O)Nc2ccn(-c3ccnc(F)c3)n2)n1] | D | 50 | RND | 301.27 | 1H NMR (400 MHz, DMSO-d₆) δ 11.23 (s, 1H), 8.68 (d, J = 2.8 Hz, 1H), 8.30 (d, J = 5.7 Hz, 1H), 7.75 (d, J = 5.7 Hz, 1H), 7.62 (d, J = 2.2 Hz, 1H), |
| | | | | | | 7.53 (d, J = 1.8 Hz, 1H), |
| | | | | | | 6.89 (d, J = 2.8 Hz, 1H), |
| | | | | | | 6.04 (6, J = 2.2 Hz, 1H), |
| | | | | | | 4.95 (s, 2H), 2.15 (s, 3H). |
| 285 | 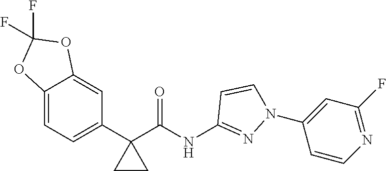 [SMILES: O=C(Nc1ccn(-c2ccnc(F)c2)n1)C1(c2ccc3c(c2)OC(F)(F)O3)CC1] | D | 26 | 0.040 | 403.23 | 1H NMR (400 MHz, DMSO-d₆) δ 9.82 (s, 1H), 8.64 (d, J = 2.8 Hz, 1H), 8.26 (d, J = 5.7 Hz, 1H), 7.72 (m, 1H), 7.50 (m, 2H), 7.38 (m, 1H), 7.28 (m, 1H), 6.89 (d, J = 2.8 Hz, 1H), 1.51 (m, 2H), 1.17 (m, 2H). |
| 286 | 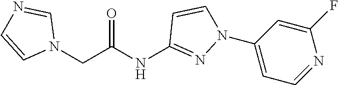 [SMILES: O=C(Cn1ccnc1)Nc1ccn(-c2ccnc(F)c2)n1] | D | 18 | 5.0 | 287.30 | 1H NMR (400 MHz, DMSO-d₆) δ 8.64 (6, J = 2.8 Hz, 1H), 8.28 (d, J = 5.8 Hz, 1H), 7.73 (d, J = 5.8 Hz, 1H), 7.62 (s, 1H), 7.50 (s, 1H), 7.15 (s, 1H), 6.89 (m, |
| | | | | | | 2H), 4.89 (s, 2H). |
| 287 | 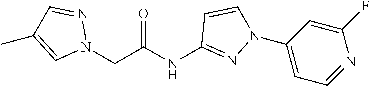 [SMILES: Cc1cnn(CC(=O)Nc2ccn(-c3ccnc(F)c3)n2)c1] | D | 37 | RND | 301.34 | 1H NMR (400 MHz, DMSO-d₆) δ 11.22 (s, 1H), 8.68 (d, J = 2.8 Hz, 1H), 8.30 (d, J = 5.7 Hz, 1H), 7.75 (d, J = 5.7 Hz, 1H), 7.57-7.46 (m, 2H), 7.26 |
| | | | | | | (s, 1H), 6.89 (d, J = 2.8 Hz, |
| | | | | | | 1H), 4.98 (s, 2H), 2.02 (s, |
| | | | | | | 3H). |
| 288 | 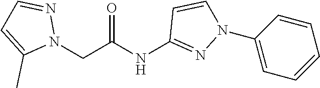 [SMILES: Cc1ccnn1CC(=O)Nc1ccn(-c2ccccc2)n1] | D | 61 | RND | 282.29 | 1H NMR (400 MHz, DMSO-d₆) δ 11.11 (s, 1H), 8.43 (6, J = 2.6 Hz, 1H), 7.78 (d, J = 8.0 Hz, 2H), 7.50 (m, 2H), 7.36-7.24 (m, 2H), 6.74 (d, J = 2.6 |
| | | | | | | Hz, 1H), 6.05 (s, 1H), 4.98 |
| | | | | | | (s, 2H), 2.25 (s, 3H). |
| 289 | 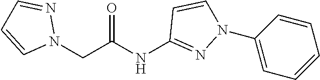 [SMILES: O=C(Cn1cccn1)Nc1ccn(-c2ccccc2)n1] | D | 41 | >5 | 268.32 | 1H NMR (400 MHz, DMSO-d₆) δ 11.10 (s, 1H), 8.43 (d, J = 2.6 Hz, 1H), 7.84-7.72 (m, 3H), 7.55- 7.43 (m, 3H), 7.28 (m, 1H), 6.75 (d, J = 2.6 Hz, 1H), |
| | | | | | | 6.28 (m, 1H), 5.05 (s, 2H). |
| 290 | 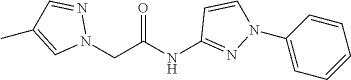 [SMILES: Cc1cnn(CC(=O)Nc2ccn(-c3ccccc3)n2)c1] | D | 18 | >5 | 282.29 | 1H NMH (400 MHz, DMS0-d₆) δ 11.05 (s, 1H), 8.42 (d, J = 2.6 Hz, 1H), 7.77 (d, J = 8.0 Hz, 2H), 7.57-7.44 (m, 3H), 7.34- 7.21 (m, 2H), 6.74 (d, J = |
| | | | | | | 2.6 Hz, 1H), 4.95 (s, 2H), |
| | | | | | | 2.03 (s, 3H). |
| 291 | 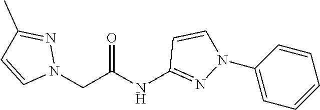 [SMILES: Cc1ccn(CC(=O)Nc2ccn(-c3ccccc3)n2)n1] | D | 37 | >5 | 282.29 | 1H NMR (400 MHz, DMSO-d₆) δ 11.06 (s, 1H), 8.42 (d, J = 2.6 Hz, 1H), 7.77 (d, J = 8.0 Hz, 2H), 7.62 (d, J = 2.1 Hz, 1H), 7.50 (m, 2H), 7.29 (m, 1H), 6.75 (d, J = 2.6 Hz, 1H), 6.05 (d, J = 2.1 Hz, 1H), |
| | | | | | | 4.93 (s, 2H), 2.15 (s, 3H). |
| 292 | 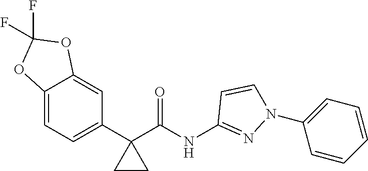 [SMILES: O=C(Nc1ccn(-c2ccccc2)n1)C1(c2ccc3c(c2)OC(F)(F)O3)CC1] | D | 39 | 0.085 | 384.30 | 1H NMR (400 MHz, DMSO-d₆) δ 9.59 (s, 1H), 8.38 (d, J = 2.6 Hz, 1H), 7.73 (d, J = 8.1 Hz, 2H), 7.51-7.19 (m, 6H), 6.73 (d, J = 2.6 Hz, 1H), 1.49 (m, 2H), 1.14 (m, 2H). |
| 293 | 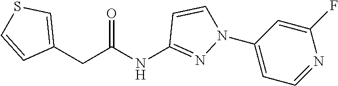 [SMILES: O=C(Cc1ccsc1)Nc1ccn(-c2ccnc(F)c2)n1] | D | 37 | 0.98 | 303.20 | 1H NMR (400 MHz, DMSO-d₆) δ 11.10 (s, 1H), 8.66 (d, J = 2.8 Hz, 1H), 8.29 (d, J = 5.7 Hz, 1H), 7.78-7.68 (m, 1H), 7.52- 7.43 (m, 2H), 7.09 (m, 1H), 6.92 (d, J = 2.8 Hz, 1H), |
| | | | | | | 3.70 (s, 2H). |
| 294 | 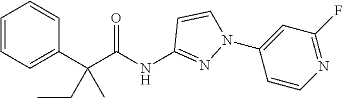 [SMILES: CCC(C)(C(=O)Nc1ccn(-c2ccnc(F)c2)n1)c1ccccc1] | D | 3 | 0.39 | 339.31 | 1H NMR (400 MHz, DMSO-d₆) δ 10.14 (s, 1H), 8.65 (d, J = 2.8 Hz, 1H), 8.26 (d, J = 5.7 Hz, 1H), 7.75-7.65 (m, 1H), 7.48 (d, J = 1.7 Hz, 1H), 7.35- 7.24 (m, 5H), 6.98 (d, J = |
| | | | | | | 2.8 Hz, 1H), 2.22 (m, 1H), |
| | | | | | | 1.93 (m, 1H), 1.53 (s, 3H), |
| | | | | | | 0.78 (t, J = 7.3 Hz, 3H). |
| 295 | 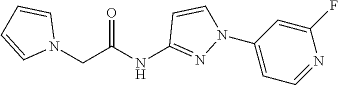 [SMILES: O=C(Cn1cccc1)Nc1ccn(-c2ccnc(F)c2)n1] | D | 22 | >5 | 286.24 | 1H NMR (400 MHz, DMSO-d₆) δ 11.15 (s, 1H), 8.68 (d, J = 2.8 Hz, 1H), 8.30 (d, J = 5.7 Hz, 1H), 7.75 (d, J = 5.7 Hz, 1H), 7.53 (d, J = 1.7 Hz, 1H), 6.91 (d, J = 2.8 Hz, 1H), |
| | | | | | | 6.77 (m, 2H), 6.01 (m, 2H), |
| | | | | | | 4.80 (s, 2H). |
| 296 | 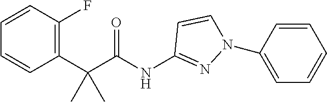 [SMILES: CC(C)(C(=O)Nc1ccn(-c2ccccc2)n1)c1ccccc1F] | D | 2 | 1.7 | 324.30 | 1H NMR (400 MHz, DMSO-d₆) δ 9.98 (s, 1H), 8.39 (d, J = 2.6 Hz, 1H), 7.77-7.68 (m, 2H), 7.50- 7.39 (m, 3H), 7.35 (d, J = 7.1 Hz, 1H), 7.24 (m, 2H), 7.17-7.11 (m, 1H), 6.81 |
| | | | | | | (d, J = 2.6 Hz, 1H), 1.56 (s, |
| | | | | | | 6H). |
| 297 | 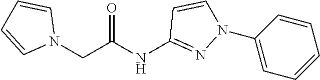 [SMILES: O=C(Cn1cccc1)Nc1ccn(-c2ccccc2)n1] | D | 37 | 1.8 | 267.26 | 1H NMR (400 MHz, DMSO-d₆) δ 10.99 (s, 1H), 8.43 (d, J = 2.6 Hz, 1H), 7.84-7.71 (m, 2H), 7.56- 7.44 (m, 2H), 7.34-7.22 (m, 1H), 6.77 (m, 3H), 6.02 |
| | | | | | | (t, J = 2.1 Hz, 2H), 4.77 (s, |
| | | | | | | 2H). |
| 298 | 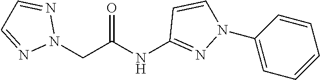 [SMILES: O=C(Cn1nccn1)Nc1ccn(-c2ccccc2)n1] | D | 51 | >2 | 269.42 | 1H NMR (400 MHz, DMSO-d₆) δ 11.26 (s, 1H), 8.44 (d, J = 2.6 Hz, 1H), 7.85 (s, 2H), 7.80-7.76 (m, 2H), 7.53-7.44 (m, 2H), 7.35-7.23 (m, 1H), |
| | | | | | | 6.74 (d, J = 2.6 Hz, 1H), |
| | | | | | | 5.40 (s, 2H). |
| 299 | 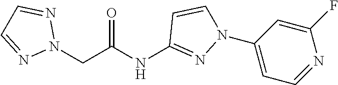 [SMILES: O=C(Cn1nccn1)Nc1ccn(-c2ccnc(F)c2)n1] | D | 6 | 5.0 | 288.22 | 1H NMR (400 MHz, DMSO-d₆) δ 11.42 (s, 1H), 8.70 (d, J = 2.8 Hz, 1H), 8.31 (d, J = 5.7 Hz, 1H), 7.85 (s, 2H), 7.76 (d, J = 5.8 Hz, 1H), 7.54 (m, 1H), |
| | | | | | | 6.88 (d, J = 2.7 Hz, 1H), |
| | | | | | | 5.42 (s, 2H). |
| 300 | 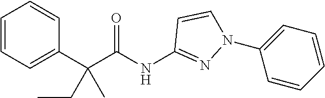 [SMILES: CCC(C)(C(=O)Nc1ccn(-c2ccccc2)n1)c1ccccc1] | D | 2 | 0.56 | 320.30 | 1H NMR (400 MHz, DMSO-d₆) δ 9.93 (s, 1H), 8.39 (d, J = 2.6 Hz, 1H), 7.78-7.68 (m, 2H), 7.50- 7.41 (m, 2H), 7.39-7.29 (m, 4H), 7.24 (m, 2H), 6.83 (d, J = 2.6 Hz, 1H), 2.22 |
| | | | | | | (m, 1H), 1.93 (m, 1H), 1.53 |
| | | | | | | (s, 3H), 0.78 (t, J = 7.3 Hz, |
| | | | | | | 3H). |
| 301 | 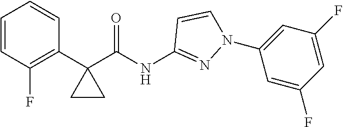 [SMILES: O=C(Nc1ccn(-c2cc(F)cc(F)c2)n1)C1(c2ccccc2F)CC1] | O | 73 | 0.14 | 358.10 | 1H NMR (400 MHz, DMSO-d₆) δ 9.48 (s, 1H), 8.47 (d, J = 2.7 Hz, 1H), 7.60-7.35 (m, 4H), 7.21 (dd, J = 12.5, 7.6 Hz, 2H), 7.17-6.98 (m, 1H), 6.78 (d, J = 2.6 Hz, 1H), 1.60 (q, J = 4.4 Hz, 2H), 1.16 (q, J = 4.3 Hz, 2H). |
| 302 | 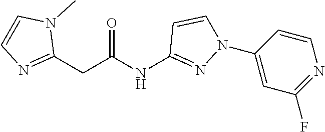 [SMILES: Cn1ccnc1CC(=O)Nc1ccn(-c2ccnc(F)c2)n1] | D | 5 | >5 | 301.29 | 1H NMR (400 MHz, DMSO-d₆) δ 11.51 (s, 1H), 8.71 (d, J = 2.8 Hz, 1H), 8.32 (d, J = 5.7 Hz, 1H), 7.82-7.50 (m, 4H), 6.88 (d, J = 2.8 Hz, 1H), 4.34 (s, 2H), 3.81 (s, 3H). |
| 303 | 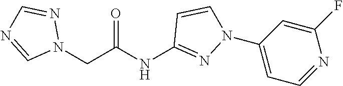 [SMILES: O=C(Cn1cncn1)Nc1ccn(-c2ccnc(F)c2)n1] | D | 6 | >5 | 288.25 | 1H NMR (400 MHz, DMSO-d₆) δ 11.41 (s, 1H), 8.70 (d, J = 2.8 Hz, 1H), 8.57 (s, 1H), 8.31 (d, J = 5.7 Hz, 1H), 8.01 (s, 1H), 7.76 (d, J = 5.7 Hz, 1H), |
| | | | | | | 7.53 (s, 1H), 6.89 (d, J = |
| | | | | | | 2.7 Hz, 1H), 5.20 (s, 2H). |
| 304 | 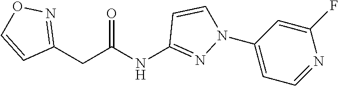 [SMILES: O=C(Cc1ccon1)Nc1ccn(-c2ccnc(F)c2)n1] | D | 9 | >5 | 288.22 | 1H NMR (400 MHz, DMSO-d₆) δ 11.26 (s, 1H), 8.86 (d, J = 1.6 Hz, 1H), 8.68 (d, J = 2.8 Hz, 1H), 8.30 (d, J = 5.7 Hz, 1H), 7.75 (m, 1H), 7.52 (d, J = |
| | | | | | | 1.7 Hz, 1H), 6.92 (d, J = 2.8 |
| | | | | | | Hz, 1H), 6.59 (d, J = 1.7 Hz, |
| | | | | | | 1H), 3.87 (s, 2H). |
| 305 | 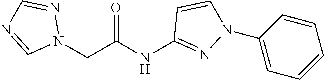 [SMILES: O=C(Cn1cncn1)Nc1ccn(-c2ccccc2)n1] | D | 4 | RND | 269.24 | 1H NMR (400 MHz, DMSO-d₆) δ 11.27 (s, 1H), 8.71 (s, 1H), 8.44 (d, J = 2.6 Hz, 1H), 8.12 (s, 1H), 7.83-7.72 (m, 2H), 7.57- 7.45 (m, 2H), 7.29 (m, 1H), |
| | | | | | | 6.74 (d, J = 2.5 Hz, 1H), |
| | | | | | | 5.20 (s, 2H). |
| 306 | 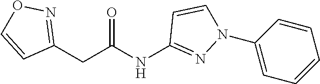 [SMILES: O=C(Cc1ccon1)Nc1ccn(-c2ccccc2)n1] | D | 6 | >2 | 269.24 | 1H NMR (400 MHz, DMSO-d₆) δ 11.10 (s, 1H), 8.86 (d, J = 1.6 Hz, 1H), 8.42 (d, J = 2.6 Hz, 1H), 7.81-7.72 (m, 2H), 7.53- 7.44 (m, 2H), 7.28 (m, 1H), |
| | | | | | | 6.77 (d, J = 2.5 Hz, 1H), |
| | | | | | | 6.59 (d, J = 1.6 Hz, 1H), |
| | | | | | | 3.84 (s, 2H). |
| 307 | 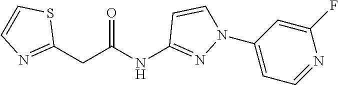 [SMILES: O=C(Cc1nccs1)Nc1ccn(-c2ccnc(F)c2)n1] | D | 34 | >2 | 304.20 | 1H NMR (400 MHz, DMSO-d₆) δ 11.30 (s, 1H), 8.69 (d, J = 2.8 Hz, 1H), 8.30 (d, J = 5.7 Hz, 1H), 7.79-7.70 (m, 2H), 7.67 (m, 1H), 7.53 (d, J = 1.8 |
| | | | | | | Hz, 1H), 6.94 (d, J = 2.8 Hz, |
| | | | | | | 1H), 4.21 (s, 2H). |
| 308 | 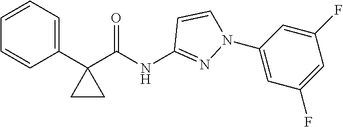 [SMILES: O=C(Nc1ccn(-c2cc(F)cc(F)c2)n1)C1(c2ccccc2)CC1] | Q | 7 | 0.14 | 340.60 | 1H NMR (400 MHz, DMSO-d₆) δ 9.37 (s, 1H), 8.48 (d, J = 2.7 Hz, 1H), 7.50 (d, J = 6.9 Hz, 2H), 7.45 (m, 1H), 7.39 (t, J = 7.4 Hz, 2H), 7.36-7.27 (m, 2H), 7.11 (s, 1H), 6.81 (d, J = 2.5 Hz, 1H), 1.48 (q, J = |
| | | | | | | 4.3 Hz, 2H), 1.13 (q, J = 4.2 |
| | | | | | | Hz, 2H). |
| 309 | 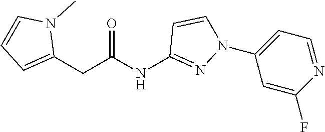 [SMILES: Cn1cccc1CC(=O)Nc1ccn(-c2ccnc(F)c2)n1] | D | 31 | >5 | 300.28 | 1H NMR (400 MHz, DMSO-d₆) δ 11.02 (s, 1H), 8.66 (d, J = 2.8 Hz, 1H), 8.29 (d, J = 5.7 Hz, 1H), 7.74 (d, J = 5.7 Hz, 1H), 7.51 (d, J = 1.8 Hz, 1H), 6.91 (d, J = 2.7 Hz, 1H), 6.65 (m, 1H), 5.94-5.84 (m, 2H), 3.69 (s, 2H), 3.56 |
| | | | | | | (s, 3H). |
| 310 | 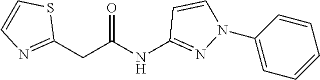 [SMILES: O=C(Cc1nccs1)Nc1ccn(-c2ccccc2)n1] | D | 37 | RND | 285.23 | 1H NMR (400 MHz, DMSO-d₆) δ 11.14 (s, 1H), 8.43 (d, J = 2.6 Hz, 1H), 7.82-7.71 (m, 3H), 7.66 (d, J = 3.3 Hz, 1H), 7.54- 7.44 (m, 2H), 7.28 (m, 1H), |
| | | | | | | 6.79 (d, J = 2.6 Hz, 1H), |
| | | | | | | 4.19 (s, 2H). |
| 311 | 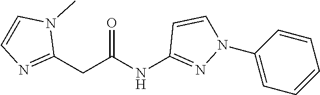 [SMILES: Cn1ccnc1CC(=O)Nc1ccn(-c2ccccc2)n1] | D | 14 | >5 | 282.24 | 1H NMR (400 MHz, DMSO-d₆) δ 11.10 (s, 1H), 8.38 (d, J = 2.6 Hz, 1H), 7.76 (d, 7 = 8.0 Hz, 2H), 7.49 (m, 2H), 7.28 (m, 1H), 7.09 (s, 1H), 6.84-6.71 (m, 2H), 3.86 (s, 2H), 3.61 |
| | | | | | | (s, 3H). |
| 312 | 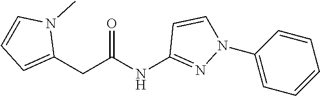 [SMILES: Cn1cccc1CC(=O)Nc1ccn(-c2ccccc2)n1] | D | 34 | RND | 281.27 | 1H NMR (400 MHz, DMSO-d₆) δ 10.85 (s, 1H), 8.40 (d, J = 2.6 Hz, 1H), 7.87-7.72 (m, 2H), 7.55- 7.43 (m, 2H), 7.27 (m, 1H), 6.77 (d, J = 2.6 Hz, 1H), 6.65 (m, 1H), 5.90 (m, 2H), |
| | | | | | | 3.67 (s, 2H), 3.58 (s, 3H). |
| 313 | 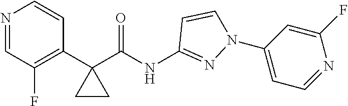 [SMILES: O=C(Nc1ccn(-c2ccnc(F)c2)n1)C1(c2ccncc2F)CC1] | D | 20 | 1.7 | 342.04 | 1H NMR (400 MHz, DMSO-d₆) δ 10.21 (s, 1H), 8.62 (d, J = 2.8 Hz, 1H), 8.52 (d, J = 1.8 Hz, 1H), 8.41 (d, J = 4.9 Hz, 1H), 8.26 (d, J = 5.7 Hz, 1H), 7.76-7.66 (m, 1H), 7.53- 7.41 (m, 2H), 6.87 (d, J = |
| | | | | | | 2.8 Hz, 1H), 1.65 (m, 2H), |
| | | | | | | 1.27 (m, 2H). |
| 314 | 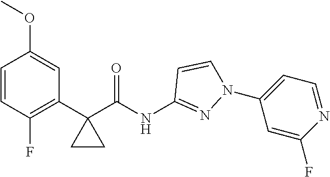 [SMILES: COc1ccc(F)c(C2(C(=O)Nc3ccn(-c4ccnc(F)c4)n3)CC2)c1] | M | 32 | 0.057 | 371.32 | 1H NMR (300 MHz, CDCl₃) δ 8.20 (d, J = 5.7 Hz, 1H), 7.89 (m, 1H), 7.77 (s, 1H), 7.35 (m, 1H), 7.20-7.08 (m, 3H), 7.01-6.91 (m, 2H), 3.86 (s, 3H), 1.81 (m, 2H), 1.27-1.20 (m, 2H). |
| 386 | 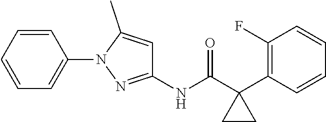 [SMILES: Cc1cc(NC(=O)C2(c3ccccc3F)CC2)nn1-c1ccccc1] | S | 56 | >5 | 336.19 | 1H NMR (400 MHz, Methanol-d₄) δ 7.53-7.32 (m, 7H), 7.28-7.12 (m, 2H), 6.56 (d, J = 0.9 Hz, 1H), 2.28 (d, J = 0.7 Hz, 3H), 1.68 (q, J = 4.1 Hz, 2H), 1.20 (q, J = 4.1 Hz, 2H). |
| 389 | 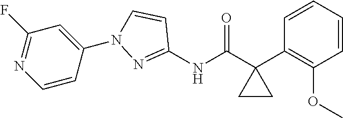 [SMILES: COc1ccccc1C1(C(=O)Nc2ccn(-c3ccnc(F)c3)n2)CC1] | E | 42 | >5 | 353.16 | 1H NMR (400 MHz, Methanol-d₄) δ 8.77 (dd, J = 4.5, 1.4 Hz, 1H), 8.51 (dd, J = 8.4, 1.4 Hz, 1H), 7.57 (dd, J = 8.4, 4.5 Hz, 1H), 7.43 (dt, J = 7.6, 1.9 Hz, 2H), 7.41-7.35 (m, 1H), 7.14-7.04 (m, 2H), |
| | | | | | | 7.00 (td, J = 7.5, 1.1 Hz, |
| | | | | | | 1H), 4.04 (s, 3H), 1.97- |
| | | | | | | 1.88 (m, 2H), 1.59-1.51 |
| | | | | | | (m, 2H). |
| 390 | 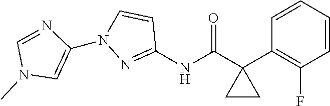 [SMILES: Cn1cnc(-n2ccc(NC(=O)C3(c4ccccc4F)CC3)n2)c1] | T | 25 | 2.1 | 326.11 | 1H NMR (300 MHz, CDCl₃) δ 7.65 (s, 1H), 7.61-7.31 (m, 5H), 7.25-7.07 (m, 2H), 6.87 (t, J = 2.1 Hz, 1H), 3.89 (d, J = 1.7 Hz, 3H), 1.80 (dd, J = 4.5, 2.6 Hz, 2H), 1.23-1.14 (m, 2H) |
| 392 | 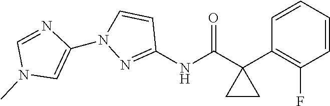 [SMILES: Cn1cnc(-n2ccc(NC(=O)C3(c4ccccc4F)CC3)n2)c1] | U | 47 | >5 | 326.10 | 1H NMR (400 MHz, CDCl₃) δ 7.90 (dd, J = 2.6, 0.6 Hz, 1H), 7.68 (s, 1H), 7.52- 7.33 (m, 2H), 7.32-7.08 (m, 3H), 6.88 (dd, J = 4.5, 2.1 Hz, 2H), 3.68 (d, J = 0.5 Hz, 3H), 1.81 (q, J = 4.1 Hz, 2H), 1.26-1.08 (m, 2H). |
| 393 | 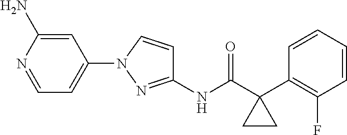 [SMILES: Nc1cc(-n2ccc(NC(=O)C3(c4ccccc4F)CC3)n2)ccn1] | V | 4 | 1.9 | 338.15 | 1H NMR (400 MHz, DMSO-d₆) δ 9.33 (s, 1H), 8.37 (d, J = 2.5 Hz, 1H), 7.90 (d, J = 5.7 Hz, 1H), 7.54-7.38 (m, 2H), 7.27- 7.15 (m, 2H), 6.87 (dd, J = 5.8, 2.0 Hz, 1H), 6.74 (d, J = 2.5 Hz, 2H), 6.10 (s, 2H), 1.60 (m, 2H), 1.16 (m, 2H). |
| 394 | 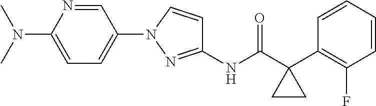 [SMILES: CN(C)c1ccc(-n2ccc(NC(=O)C3(c4ccccc4F)CC3)n2)cn1] | V | 4 | >5 | 366.18 | 1H NMR (400 MHz, DMSO-d₆) δ 9.57 (s, 1H), 8.53 (d, J = 2.7 Hz, 1H), 8.07 (d, J = 5.6 Hz, 1H), 7.53-7.33 (m, 2H), 7.25- 7.17 (m, 2H), 6.95 (dd, J = 5.7, 1.8 Hz, 1H), 6.88 (d, J = 1.8 Hz, 1H), 6.79 (d, J = |
| | | | | | | 2.7 Hz, 1H), 3.03 (s, 6H), |
| | | | | | | 1.61 (m, 2H), 1.15 (m, 2H). |
| 395 | 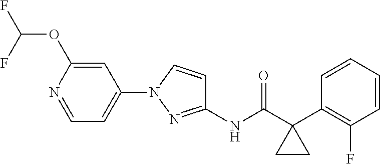 [SMILES: O=C(Nc1ccn(-c2ccnc(OC(F)F)c2)n1)C1(c2ccccc2F)CC1] | V | 27 | >5 | 389.14 | 1H NMR (400 MHz, DMSO-d₆) δ 9.65 (s, 1H), 8.63 (d, J = 2.8 Hz, 1H), 8.27 (d, J = 5.8 Hz, 1H), 7.94-7.51 (m, 2H), 7.51- 7.38 (m, 3H), 7.25-7.15 (m, 2H), 6.86 (d, J = 2.7 Hz, 1H), 1.61 (q, J = 4.3 Hz, 2H), 1.17 (q, J = 4.4 Hz, 2H). |
| 396 | 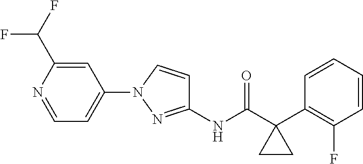 [SMILES: O=C(Nc1ccn(-c2ccnc(C(F)F)c2)n1)C1(c2ccccc2F)CC1] | V | 44 | >5 | 373.11 | 1H NMR (400 MHz, DMSO-d₆) δ 9.68 (s, 1H), 8.69 (m, 2H), 8.04 (d, J = 2.1 Hz, 1H), 7.92 (dd, J = 5.6, 2.1 Hz, 1H), 7.51- 7.37 (m, 2H), 7.28-7.17 (m, 2H), 6.98 (s, 1H), 6.88 (d, J = 2.7 Hz, 1H), 1.62 (m, 2H), 1.17 (m, 2H). |
| 397 | 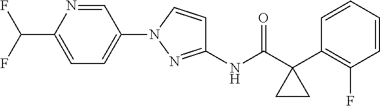 [SMILES: O=C(Nc1ccn(-c2ccc(C(F)F)nc2)n1)C1(c2ccccc2F)CC1] | V | 55 | >5 | 373.17 | 1H NMR (400 MHz, DMSO-d₆) δ 9.60 (s, 1H), 9.12 (d, J = 2.5 Hz, 1H), 8.58 (d, J = 2.8 Hz, 1H), 8.31 (dd, J = 8.6, 2.6 Hz, 1H), 7.80 (d, J = 8.6 Hz, 1H), 7.51-7.35 (m, 2H), |
| | | | | | | 7.27-7.18 (m, 2H), 6.86- |
| | | | | | | 6.82 (m, 1H), 1.62 (q, J = |
| | | | | | | 4.3 Hz, 2H), 1.17 (q, J = 4.3 |
| | | | | | | Hz, 2H). |
| 399 | 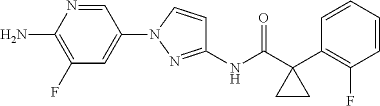 [SMILES: Nc1ncc(-n2ccc(NC(=O)C3(c4ccccc4F)CC3)n2)cc1F] | V | 14 | 0.28 | 356.15 | 1H NMR (400 MHz, DMSO-d₆) δ 9.28 (s, 1H), 8.18 (dd, J = 12.5, 2.4 Hz, 2H), 7.78 (dd, J = 12.0, 2.3 Hz, 1H), 7.54-7.35 (m, 2H), 7.27-7.15 (m, 2H), 6.67 (d, J = 2.5 Hz, 1H), 6.35 (s, 2H), 1.59 (m, 2H), |
| | | | | | | 1.14 (m, 2H). |
| 400 | 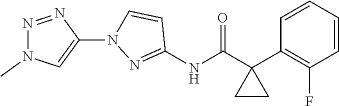 [SMILES: Cn1cc(-n2ccc(NC(=O)C3(c4ccccc4F)CC3)n2)nn1] | W | 86 | >5 | 327.10 | 1H NMR (400 MHz, CDCl₃) δ 8.06 (dd, J = 2.7, 0.6 Hz, 1H), 7.70 (s, 1H), 7.56 (s, 1H), 7.52-7.37 (m, 2H), 7.27-6.99 (m, 2H), 6.94 (d, J = 2.6 Hz, 1H), 4.10 (s, 3H), 1.81 (q, J = 4.1 Hz, 2H), 1.20 (q, J = 4.1 Hz, |
| | | | | | | 2H). |
| 407 | 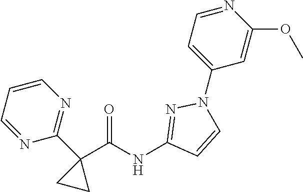 [SMILES: COc1cc(-n2ccc(NC(=O)C3(c4ncccn4)CC3)n2)ccn1] | AD | 31 | >5 | 337.14 | 1H NMR (400 MHz, DMSO-d6) δ 12.13 (s, 1H), 8.82 (d, J = 4.9 Hz, 2H), 8.62 (d, J = 2.8 Hz, 1H), 8.21 (d, J = 5.8 Hz, 1H), 7.48-7.38 (m, 2H), 7.19 (d, J = 1.9 Hz, 1H), 6.93 (d, J = 2.7 Hz, 1H), 3.89 (s, 3H), 1.68 (p, J = 3.7, 3.0 Hz, 2H), 1.62 (q, J = 4.3, 3.5 Hz, 2H). |
| 408 | 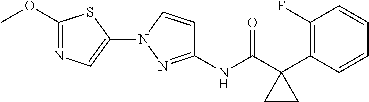 [SMILES: COc1ncc(-n2ccc(NC(=O)C3(c4ccccc4F)CC3)n2)s1] | V | 3 | 1.0 | 359.07 | 1H NMR (400 MHz, DMSO-d₆) δ 9.97 (s, 1H), 8.32 (d, J = 2.8 Hz, 1H), 7.51-7.36 (m, 3H), 7.23- 7.17 (m, 2H), 6.87 (d, J = 2.8 Hz, 1H), 2.42 (s, 3H), 1.62 (m, 2H). 1.17 (m, 2H). |
| 409 | 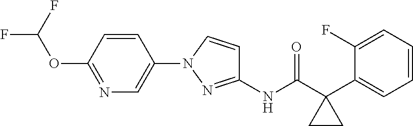 [SMILES: O=C(Nc1ccn(-c2ccc(OC(F)F)nc2)n1)C1(c2ccccc2F)CC1] | V | 86 | >5 | 389.06 | 1H NMR (400 MHz, DMSO-d₆) δ 9.49 (s, 1H), 8.65 (d, J = 2.8 Hz, 1H), 8.43 (d, J = 2.6 Hz, 1H), 8.26 (dd, J = 8.9, 2.9 Hz, 1H), 7.69 (dd, J = 72.7, 72.7 Hz, 1H), 7.50-7.38 (m, 2H), 7.21 (m, 3H), 6.78 (d, J = 2.6 Hz, 1H), 1.60 |
| | | | | | | (m, 2H), 1.15 (m, 2H). |
| 410 | 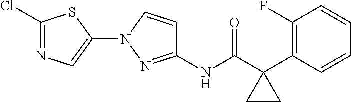 [SMILES: O=C(Nc1ccn(-c2cnc(Cl)s2)n1)C1(c2ccccc2F)CC1] | V | 18 | 0.10 | 363.01 | 1H NMR (400 MHz, DMSO-d₆) δ 9.74 (s, 1H), 8.34 (d, J = 2.7 Hz, 1H), 7.93 (s, 1H), 7.52-7.34 (m, 2H), 7.19 (m, 2H), 6.79 (d, J = 2.7 Hz, 1H), 1.59 (m, 2H), 1.15 (m, 2H). |
| 411 | 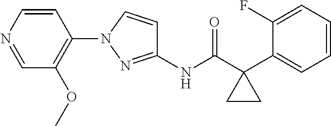 [SMILES: COc1cnccc1-n1ccc(NC(=O)C2(c3ccccc3F)CC2)n1] | V | 79 | >5 | 353.34 | 1H NMR (400 MHz, DMSO-d₆) δ 9.52 (s, 1H), 8.53 (s, 1H), 8.38 (d, J = 2.7 Hz, 1H), 8.24 (d, J = 5.2 Hz, 1H), 7.68 (d, J = 5.2 Hz, 1H), 7.54-7.34 (m, 2H), 7.21 (m, 2H), 6.79 (d, J = 2.7 Hz, 1H), 4.03 (s, 3H), 1.61 (m, 2H), 1.16 (m, 2H). |
| 412 | 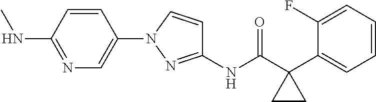 [SMILES: CNc1ccc(-n2ccc(NC(=O)C3(c4ccccc4F)CC3)n2)cn1] | V | 100 | RND | 352.14 | 1H NMR (400 MHz, DMSO-d₆) δ 9.25 (s, 1H), 8.30 (d, J = 2.7 Hz, 1H), 8.11 (d, J = 2.5 Hz, 1H), 7.69 (dd, J = 9.0, 2.8 Hz, 1H), 7.53-7.36 (m, 2H), 7.21 (dd, J = 10.3, 8.0 Hz, |
| | | | | | | 2H), 6.65 (m, 2H), 6.50 (d, |
| | | | | | | J = 9.0 Hz, 1H), 4.06 (s, |
| | | | | | | 3H), 1.58 (m, 2H), 1.13 (m, |
| | | | | | | 2H). |
| 413 | 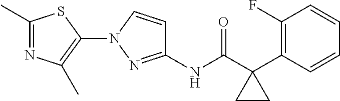 [SMILES: Cc1nc(C)c(-n2ccc(NC(=O)C3(c4ccccc4F)CC3)n2)s1] | V | 13 | >5 | 357.09 | 1H NMR (400 MHz. DMSO-d₆) δ 9.50 (s, 1H), 7.96 (d, J = 2.5 Hz, 1H), 7.41 (m, 2H), 7.22-7.13 (m, 2H), 6.71 (d, J = 2.5 Hz, 1H), 4.06 (s, 3H), 2.25 (s, 3H), 1.58 (m, 2H), 1.14 |
| | | | | | | (m, 2H). |
| 414 | 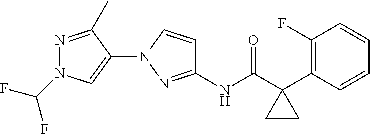 [SMILES: Cc1nn(C(F)F)cc1-n1ccc(NC(=O)C2(c3ccccc3F)CC2)n1] | U | 40 | >5 | 376.07 | 1H NMR (400 MHz, CDCl₃) δ 7.87 (s, 1H), 7.63 (s, 1H), 7.48-7.41 (m, 2H), 7.42- 7.33 (m, 1H), 7.23-7.05 (m, 3H), 6.95-6.90 (m, 1H), 2.30 (q, J = 0.8 Hz, 3H), 1.83-1.76 (m, 2H), 1.23-1.15 (m, 2H). |
| 415 | 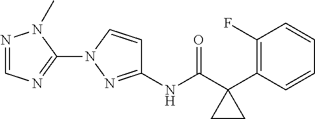 [SMILES: Cn1ncnc1-n1ccc(NC(=O)C2(c3ccccc3F)CC2)n1] | U | 30 | 0.090 | 327.13 | 1H NMR (400 MHz, CDCl₃) δ 8.05 (dd, J = 2.7, 0.6 Hz, 1H), 7.70 (s, 1H), 7.61 (s, 1H), 7.51-738 (m, 2H), 7.26-7.21 (m, 1H), 7.20- 7.14 (m, 1H), 7.01 (d, J = 2.8 Hz, 1H), 4.04 (s, 3H), 1.85-1.78 (m, 2H), 1.26- |
| | | | | | | 1.19 (m, 2H). |
| 416 | 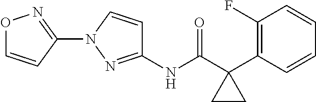 [SMILES: O=C(Nc1ccn(-c2ccon2)n1)C1(c2ccccc2F)CC1] | U | 40 | RND | 313.54 | 1H NMR (400 MHz, CDCl₃) δ 9.40 (s, 1H), 8.47-8.36 (m, 1H), 7.83 (d, J = 2.3 Hz, 1H), 7.63 (d, J = 4.9 Hz, 1H), 7.57-7.42 (m, 2H), 7.35-7.17 (m, 2H), 6.56 (d, J = 2.3 Hz, 1H), 1.93- |
| | | | | | | 1.84 (m, 2H), 1.39-1.30 |
| | | | | | | (m, 2H). |
| 417 | 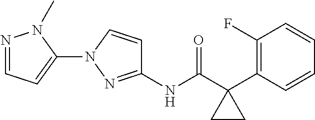 [SMILES: Cn1nccc1-n1ccc(NC(=O)C2(c3ccccc3F)CC2)n1] | X | 38 | 0.29 | 326.41 | 1H NMR (400 MHz, CDCl₃) δ 7.66 (s, 1H), 7.52-7.33 (m, 4H), 7.23-7.07 (m, 2H), 6.98 (d, J = 2.5 Hz, 1H), 6.22 (d, J = 2.0 Hz, 1H), 3.73 (s, 3H), 1.80 (q, J = 4.1 Hz, 2H), 1.22-1.19 (m, 2H). |
| 424 | 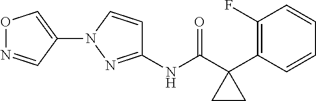 [SMILES: O=C(Nc1ccn(-c2cnoc2)n1)C1(c2ccccc2F)CC1] | X | 26 | >5 | 312.78 | 1H NMR (400 MHz, CDCl₃) δ 9.40 (s, 1H), 8.41 (dd, J = 5.0, 0.5 Hz, 1H), 7.83 (d, J = 2.3 Hz, 1H), 7.63 (d, J = 5.0 Hz, 1H), 7.58-7.41 (m, 2H), 7.37-7.17 (m, 3H), 6.56 (d, J = 2.4 Hz, 1H), |
| | | | | | | 1.93-1.82 (m, 2H), 1.42- |
| | | | | | | 1.30 (m, 2H). |
| 425 | 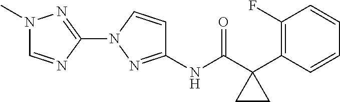 [SMILES: Cn1cnc(-n2ccc(NC(=O)C3(c4ccccc4F)CC3)n2)n1] | X | 28 | >5 | 327.13 | 1H NMR (400 MHz, CDCl₃) δ 8.06 (dd, J = 2.7, 0.6 Hz, 1H), 7.90 (d, J = 0.7 Hz, 1H), 7.84 (s, 1H), 7.44- 7.33 (m, 2H), 7.21-7.08 (m, 2H), 6.99 (d, J = 2.7 Hz, 1H), 3.88 (d, J = 0.6 Hz, |
| | | | | | | 3H), 1.79 (t, J = 3.6 Hz, |
| | | | | | | 2H), 1.18 (q, J = 4.0 Hz, |
| | | | | | | 2H). |
| 418 | 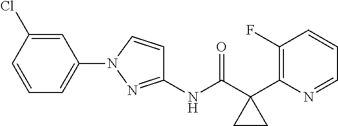 [SMILES: O=C(Nc1ccn(-c2cccc(Cl)c2)n1)C1(c2ncccc2F)CC1] | Y | 23 | 0.10 | 357.05 | 1H NMR (400 MHz, Methanol-d₄) δ 8.39 (dt, J = 4.7, 1.4 Hz, 1H), 8.14 (d, J = 2.7 Hz, 1H), 7.73 (t, J = 2.1 Hz, 1H), 7.67-7.55 (m, 2H), 7.48-7.32 (m, 2H), 7.22 (ddd, J = 8.1, 2.0, 0.9 Hz, 1H), 6.80 (d, J = 2.7 Hz, |
| | | | | | | 1H), 1.76-1.64 (m, 2H), |
| | | | | | | 1.57-1.40 (m, 2H). |
| 419 | 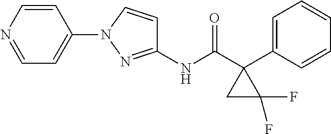 [SMILES: O=C(Nc1ccn(-c2ccncc2)n1)C1(c2ccccc2)CC1(F)F] | Y | 23 | 0.24 | 341.10 | 1H NMR (400 MHz, Methanol-d₄) δ 8.57-8.49 (m, 2H), 8.33 (d, J = 2.8 Hz, 1H), 7.80-7.74 (m, 2H), 7.65-7.56 (m, 2H), 7.50-7.34 (m, 3H), 6.92 (d, J = 2.8 Hz, 1H), 5.48 (s, 0H), 2.49 (ddd, J = 13.6, 8.4, 5.3 Hz, 1H), 2.02 (ddd, |
| | | | | | | J = 11.9, 8.4, 5.4 Hz, 1H). |
| 420 | 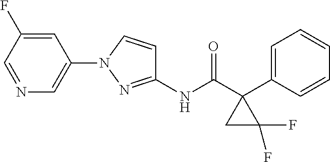 [SMILES: O=C(Nc1ccn(-c2cncc(F)c2)n1)C1(c2ccccc2)CC1(F)F] | Y | 31 | 0.10 | 359.06 | 1H NMR (400 MHz, Methanol-d₄) δ 8.84 (dd, J = 2.2, 0.9 Hz, 1H), 8.33 (d, J = 2.5 Hz, 1H), 8.25 (d, J = 2.8 Hz, 1H), 8.00 (dt, J = 9.9, 2.3 Hz, 1H), 7.64- 7.56 (m, 2H), 7.47-7.33 (m, 3H), 6.87 (d, J = 2.7 Hz, 1H), 2.49 (ddd, J = 13.6, 8.4, 5.3 Hz, 1H), 2.02 (ddd, J = 11.9, 8.4, 5.5 Hz, |
| | | | | | | 1H). |
| 421 | 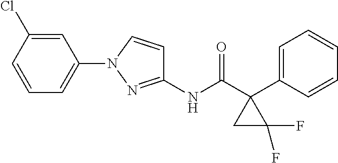 [SMILES: O=C(Nc1ccn(-c2cccc(Cl)c2)n1)C1(c2ccccc2)CC1(F)F] | Y | 27 | 0.47 | 374.05 | 1H NMR (400 MHz, Methanol-d₄) δ 8.13 (d, J = 2.7 Hz, 1H), 7.76 (t, J = 2.1 Hz, 1H), 7.60 (ddd, J = 8.2, 2.1, 1.0 Hz, 3H), 7.47- 7.33 (m, 4H), 7.22 (ddd, J = 8.0, 2.0, 0.9 Hz, 1H), 6.81 (d, J = 2.7 Hz, 1H), 2.50 (ddd, J = 13.6, 8.3, 5.3 Hz, 1H), 2.01 (ddd, J = 12.0, 8.4, 5.4 Hz, 1H). |
| 422 | 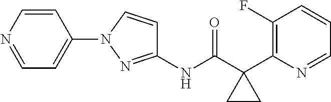 [SMILES: O=C(Nc1ccn(-c2ccncc2)n1)C1(c2ncccc2F)CC1] | Y | 11 | 0.20 | 324.07 | 1H NMR (400 MHz, Methanol-d₄) δ 8.65-8.55 (m, 2H), 8.45-8.37 (m, 2H), 7.95-7.87 (m, 2H), 7.60 (dddd, J = 22.8, 9.7, 8.4, 1.4 Hz, 1H), 7.43 (dddd, J = 25.5, 8.4, 4.8, |
| | | | | | | 4.1 Hz, 1H), 6.98 (d, J = 2.8 |
| | | | | | | Hz, 1H), 1.77-1.65 (m, |
| | | | | | | 2H), 1.55-1.41 (m, 2H). |
| 423 | 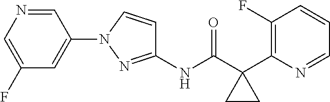 [SMILES: O=C(Nc1ccn(-c2cncc(F)c2)n1)C1(c2ncccc2F)CC1] | AC | 22 | 0.046 | 342.09 | 1H NMR (400 MHz, DMSO-d6) δ 10.26 (s, 1H), 8.93 (t, J = 1.6 Hz, 1H), 8.53 (d, J = 2.7 Hz, 1H), 8.48 (d, J = 2.5 Hz, 1H), 8.37 (dt, J = 4.7, 1.6 Hz, 1H), 8.10 (dt, J = 10.4, 2.4 Hz, 1H), 7.67 (ddd, J = |
| | | | | | | 10.5, 8.3, 1.4 Hz, 1H), 7.43 |
| | | | | | | (dt, J = 8.5, 4.4 Hz, 1H), |
| | | | | | | 6.85 (d, J = 2.7 Hz, 1H), |
| | | | | | | 1.63 (q, J = 4.2 Hz, 2H), |
| | | | | | | 1.39 (q, J = 4.2 Hz, 2H). |
| 426 | 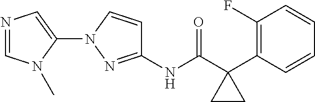 [SMILES: Cn1cncc1-n1ccc(NC(=O)C2(c3ccccc3F)CC2)n1] | U | 30 | >2 | 326.07 | 1H NMR (400 MHz, CDCl₃) δ 7.65 (s, 1H), 7.47-7.40 (m, 3H), 7.40-7.33 (m, 1H), 7.22-7.09 (m, 2H), 7.05 (d, J = 1.1 Hz, 1H), 6.95 (d, J = 2.5 Hz, 1H), 3.45 (s, 3H), 1.88-1.75 |
| | | | | | | (m, 2H), 1.22-1.15 (m, |
| | | | | | | 2H). |
| 427 | 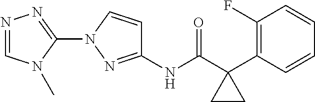 [SMILES: Cn1cnnc1-n1ccc(NC(=O)C2(c3ccccc3F)CC2)n1] | U | 8 | >2 | 327.08 | 1H NMR (400 MHz, CDCl₃) δ 8.06 (dd, J = 2.8, 0.5 Hz, 1H), 8.00 (s, 1H), 7.60 (s, 1H), 7.50-7.37 (m, 2H), 7.25-7.13 (m, 2H), 7.01 (d, J = 2.7 Hz, 1H), 3.78 (s, 3H), 1.92-1.73 (m, 2H), |
| | | | | | | 1.26-1.18 (m, 2H). |
| 428 | 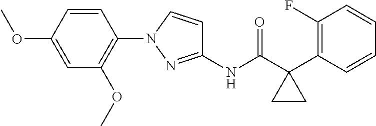 [SMILES: COc1ccc(-n2ccc(NC(=O)C3(c4ccccc4F)CC3)n2)c(OC)c1] | Z | 52 | >5 | 382.06 | 1H NMR (400 MHz, DMSO-d₆) δ 9.16 (s, 1H), 7.86 (d, J = 2.6 Hz, 1H), 7.52-7.43 (m, 1H), 7.38 (mz, 2H), 7.21 (m, 2H), 6.72 (d, J = 2.6 Hz, 1H), 6.62-6.55 (m, 2H), 3.80 (d, J = 4.3 Hz, 6H), 1.58 (m, 2H), 1.13 (m, 2H). |
| 429 | 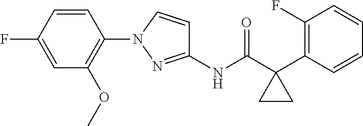 [SMILES: COc1cc(F)ccc1-n1ccc(NC(=O)C2(c3ccccc3F)CC2)n1] | Z | 56 | >5 | 370.08 | 1H NMR (400 MHz, DMSO-d₆) δ 9.23 (s, 1H), 7.96 (d, J = 2.5 Hz, 1H), 7.49 (m, 2H), 7.39 (m, 1H), 7.26-7.11 (m, 3H), 6.86 (m, 1H), 6.65 (d, J = 2.5 Hz, 1H), 3.84 (s, 3H), 1.59 (m, 2H), 1.14 (m, 2H). |
| 430 | 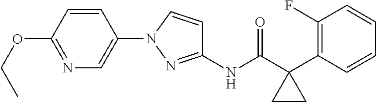 [SMILES: CCOc1ccc(-n2ccc(NC(=O)C3(c4ccccc4F)CC3)n2)cn1] | Z | 48 | >5 | 367.08 | 1H NMR (400 MHz, CDCl₃) δ 8.32 (d, J = 2.8 Hz, 1H), 7.76 (m, 2H), 7.68 (d, J = 2.5 Hz, 1H), 7.51-7.38 (m, 2H), 7.26-7.13 (m, 2H), 6.98 (d, J = 2.6 Hz, 1H), 6.79 (d, J = 9.0 Hz, 1H), |
| | | | | | | 4.38 (q, J = 7.1 Hz, 2H), |
| | | | | | | 1.82 (m, 2H), 1.42 (t, J = |
| | | | | | | 7.1 Hz, 3H), 1.22 (m, 2H). |
| 431 | 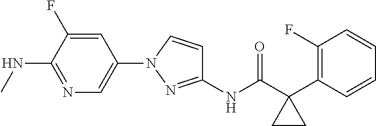 [SMILES: CNc1ncc(-n2ccc(NC(=O)C3(c4ccccc4F)CC3)n2)cc1F] | Z | 29 | 0.86 | 370.11 | 1H NMR (400 MHz, DMSO-d₆) δ 9.32 (s, 1H), 8.21 (m, 2H), 7.76 (m, 1H), 7.53-7.35 (m, 2H), 7.21 (m, 2H), 6.78 (d, J = 5.1 Hz, 1H), 6.68 (d, J = 2.5 Hz, 1H), 2.85 (d, J = 4.7 Hz, 3H), 1.59 (m, 2H), 1.14 (m, 2H). |
| 439 | 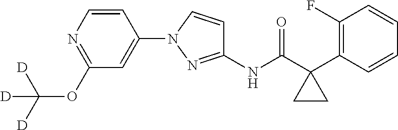 [SMILES: [2H]C([2H])([2H])Oc1cc(-n2ccc(NC(=O)C3(c4ccccc4F)CC3)n2)ccn1] | Z | 13 | 0.080 | 356.15 | 1H NMR (300 MHz, CDCl₃) δ 8.14 (dd, J = 5.8, 0.6 Hz, 1H), 7.85 (dd, J = 2.7, 0.6 Hz, 1H), 7.70 (s, 1H), 7.53- 7.40 (m, 2H), 7.26-7.15 (m, 2H), 7.08 (dd, J = 5.7, 2.0 Hz, 1H), 7.03 (d, J = 2.7 Hz, 1H), 6.88 (dd, J = 2.0, 0.6 Hz, 1H), 1.82 (q, J = 4.1 |
| | | | | | | Hz, 2H), 1.26-1.19 (m, 2H). |
| 440 | 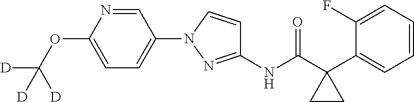 [SMILES: [2H]C([2H])([2H])Oc1ccc(-n2ccc(NC(=O)C3(c4ccccc4F)CC3)n2)cn1] | Z | 54 | 0.052 | 356.20 | 1H NMR (300 MHz, CDCl₃) δ 8.32 (dd, J = 2.8, 0.7 Hz, 1H), 7.76 (dd, J = 8.9, 2.8 Hz, 1H), 7.68 (dd, J = 2.6, 1H), 7.56-7.36 (m, 2H), 7.25-7.12 (m, 2H), 6.97 (d, J = 2.6, 1H), 6.79 (dd, |
| | | | | | | J = 8.9, 0.7 Hz, 1H), 1.85- |
| | | | | | | 1.76 (m, 2H), 1.23-1.15 |
| | | | | | | (m, 2H). |
| 441 | 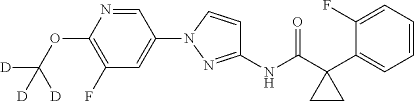 [SMILES: [2H]C([2H])([2H])Oc1ncc(-n2ccc(NC(=O)C3(c4ccccc4F)CC3)n2)cc1F] | Z | 13 | 0.044 | 371.23 | 1H NMR (300 MHz, DMSO-d₆) δ 9.43 (s, 1H), 8.37 (m, 2H), 8.12 (dd, J = 11.5, 2.4 Hz, 1H), 7.51- 7.34 (m, 2H), 7.27-7.15 (m, 2H), 6.76 (d, J = 2.6 Hz, 1H), 1.60 (m, 2H), 1.15 |
| | | | | | | (m, 2H). |
| 448 | 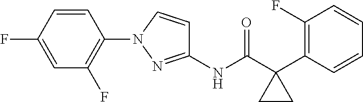 [SMILES: O=C(Nc1ccn(-c2ccc(F)cc2F)n1)C1(c2ccccc2F)CC1] | Z | 5 | 0.14 | 358.31 | 1H NMR (400 MHz, DMSO-d₆) δ 9.39 (s, 1H), 8.04 (t, J = 2.6 Hz, 1H), 7.72 (m, 1H), 7.59-7.38 (m, 3H), 7.26-7.18 (m, 3H), 6.75 (d, J = 2.6 Hz, 1H), 1.60 (m, 2H), 1.15 (m, |
| | | | | | | 2H). |
| 449 | 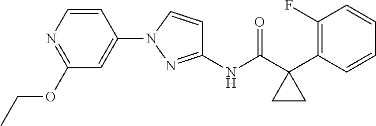 [SMILES: CCOc1cc(-n2ccc(NC(=O)C3(c4ccccc4F)CC3)n2)ccn1] | Z | 6 | >5 | 367.13 | 1H NMR (400 MHz, DMSO-d₆) δ 9.55 (s, 1H), 8.56 (d, J = 2.8 Hz, 1H), 8.15 (d, J = 5.8 Hz, 1H), 7.47 (m, 1H), 7.38 (m, 2H), 7.21 (m, 2H), 7.12 (d, J = 1.9 Hz, 1H), 6.80 (d, J = 2.8 Hz, 1H), 4.32 (q, J = 7.0 Hz, 2H), 1.61 (m, 2H), 1.31 (t, |
| | | | | | | J = 7.0 Hz, 3H), 1.16 (m, |
| | | | | | | 2H). |
| 442 | 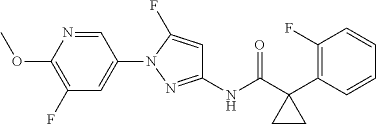 [SMILES: COc1ncc(-n2nc(NC(=O)C3(c4ccccc4F)CC3)cc2F)cc1F] | O | 62 | >5 | 389.05 | 1H NMR (400 MHz, Methanol-d₄) δ 7.89 (d, J = 2.4 Hz, 1H), 7.51 (dd, J = 11.2, 2.4 Hz, 1H), 7.40 (td, J = 7.8, 1.8 Hz, 1H), 7.24 (dddd, J = 8.2, 7.2, 5.2, 1.8 Hz, 1H), 7.13 (td, J = 7.6, 1.3 Hz, 1H), 6.98 (ddd, J = 10.9, 8.1, 1.3 Hz, 1H), 5.40 |
| | | | | | | (d, J = 5.2 Hz, 1H), 3.98 (s, |
| | | | | | | 3H), 1.84-1.76 (m, 2H), |
| | | | | | | 1.41-1.32 (m, 2H). |
| 443 | 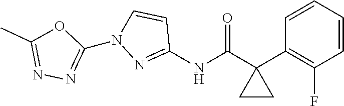 [SMILES: Cc1nnc(-n2ccc(NC(=O)C3(c4ccccc4F)CC3)n2)o1] | AA | 7 | RND | 328.43 | 1H NMR (400 MHz, CDCl₃) δ 8.05 (dd, J = 2.9, 0.6 Hz, 1H), 7.84 (s, 1H), 7.45- 7.36 (m, 2H), 7.23-7.10 (m, 3H), 2.55 (s, 3H), 1.83- 1.78 (m, 2H), 1.24-1.19 (m, 2H). |
| 450 | 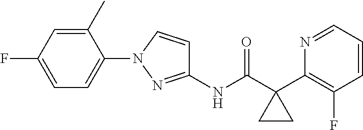 [SMILES: Cc1cc(F)ccc1-n1ccc(NC(=O)C2(c3ncccc3F)CC2)n1] | AB | 10 | >5 | 355.10 | 1H NMR (400 MHz, DMSO-d6) δ 10.01 (s, 1H), 8.36 (dt, J = 4.6, 1.5 Hz, 1H), 7.87 (d, J = 2.4 Hz, 1H), 7.67 (ddd, J = 10.2, 8.4, 1.4 Hz, 1H), 7.46- 7.31 (m, 2H), 7.25 (dd, J = 9.7, 3.0 Hz, 1H), 7.14 (td, J = 8.5, 3.0 Hz, 1H), 6.69 (d, |
| | | | | | | J = 2.4 Hz, 1H), 2.20 (s, |
| | | | | | | 3H), 1.61 (q, J = 4.2 Hz, |
| | | | | | | 2H), 1.36 (q, J = 4.2 Hz, |
| | | | | | | 2H). |
| 451 | 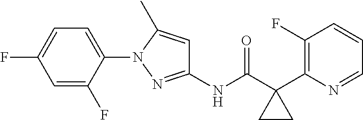 [SMILES: Cc1cc(NC(=O)C2(c3ncccc3F)CC2)nn1-c1ccc(F)cc1F] | AB | 13 | 2.4 | 373.10 | 1H NMR (400 MHz, DMSO-d6) δ 9.93 (s, 1H), 8.35 (dt, J = 4.6, 1.6 Hz, 1H), 7.71-7.51 (m, 3H), 7.40 (dt, J = 8.5, 4.4 Hz, 1H), 7.30-7.19 (m, 1H), 6.54 (s, 1H), 2.11 (s, 3H), 1.58 (d, J = 3.1 Hz, 2H), |
| | | | | | | 1.35 (d, J = 3.1 Hz, 2H). |
| 452 | 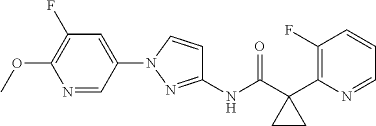 [SMILES: COc1ncc(-n2ccc(NC(=O)C3(c4ncccc4F)CC3)n2)cc1F] | AB | 12 | 0.054 | 372.07 | 1H NMR (400 MHz, DMSO-d6) δ 10.21 (s, 1H), 8.43-8.34 (m, 3H), 8.13 (dd, J = 11.4, 2.4 Hz, 1H), 7.68 (ddd, J = 10.2, 8.4, 1.4 Hz, 1H), 7.43 (dt, J = 8.5, 4.4 Hz, 1H), 6.78 (d, J = 2.6 Hz, 1H), 3.97 (s, 3H), 1.62 (q, J = 4.2 Hz, 2H), |
| | | | | | | 1.38 (q, J = 4.2 Hz, 2H). |
| 453 | 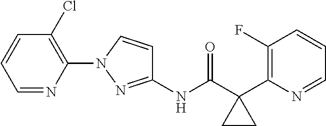 [SMILES: O=C(Nc1ccn(-c2ncccc2Cl)n1)C1(c2ncccc2F)CC1] | AB | 10 | >5 | 358.03 | 1H NMR (400 MHz, DMSO-d6) δ 10.21 (s, 1H), 8.49 (dd, J = 4.6, 1.5 Hz, 1H), 8.36 (dt, J = 4.7, 1.6 Hz, 1H), 8.21-8.14 (m, 2H), 7.67 (ddd, J = 10.1, 8.3, 1.4 Hz, 1H), 7.51 (dd, J = 8.0, 4.6 Hz, 1H), 7.41 (dt, |
| | | | | | | J = 8.5, 4.4 Hz, 1H), 6.78 |
| | | | | | | (d, J = 2.6 Hz, 1H), 1.62 (q, |
| | | | | | | J = 4.1 Hz, 2H), 1.38 (q, J = |
| | | | | | | 4.2 Hz, 2H). |
| 454 | 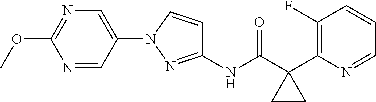 [SMILES: COc1ncc(-n2ccc(NC(=O)C3(c4ncccc4F)CC3)n2)cn1] | AB | 5 | >5 | 355.10 | 1H NMR (400 MHz, DMSO-d6) δ 10.26 (s, 1H), 8.97 (s, 2H), 8.41-8.33 (m, 2H), 7.68 (ddd, J = 10.2, 8.4, 1.4 Hz, 1H), 7.42 (dt, J = 8.5, 4.3 Hz, 1H), 6.81 (d, J = 2.6 Hz, 1H), |
| | | | | | | 3.95 (s, 3H), 1.62 (q, J = |
| | | | | | | 4.2 Hz, 2H), 1.38 (q, J = 4.2 |
| | | | | | | Hz, 2H). |
| 455 | 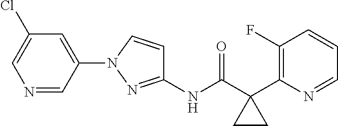 [SMILES: O=C(Nc1ccn(-c2cncc(Cl)c2)n1)C1(c2ncccc2F)CC1] | AB | 12 | 0.10 | 358.03 | 1H NMR (400 MHz, DMSO-d6) δ 10.29 (s, 1H), 9.00 (d, J = 2.3 Hz, 1H), 8.53 (dd, J = 19.6, 2.4 Hz, 2H), 8.38 (dt, J = 4.6, 1.6 Hz, 1H), 8.31 (t, J = 2.2 Hz, 1H), 7.68 (ddd, J = 10.2, 8.4, 1.4 Hz, 1H), 7.43 (dt, J = 8.5, 4.4 Hz, 1H), 6.85 (d, |
| | | | | | | J = 2.7 Hz, 1H), 1.63 (q, J = |
| | | | | | | 4.2 Hz, 2H), 1.39 (q, J = 4.2 |
| | | | | | | Hz, 2H). |
| 456 | 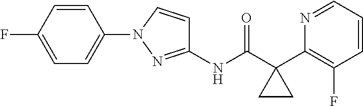 [SMILES: O=C(Nc1ccn(-c2ccc(F)cc2)n1)C1(c2ncccc2F)CC1] | AB | 11 | 0.10 | 341.10 | 1H NMR (400 MHz, DMSO-d6) δ 10.14 (s, 1H), 8.37 (dt, J = 6.6, 2.1 Hz, 2H), 7.81-7.72 (m, 2H), 7.68 (ddd, J = 10.3, 8.4, 1.4 Hz, 1H), 7.42 (dt, J = 8.5, 4.3 Hz, 1H), 7.36- |
| | | | | | | 7.27 (m, 2H), 6.74 (d, J = |
| | | | | | | 2.6 Hz, 1H), 1.62 (q, J = 4.2 |
| | | | | | | Hz, 2H), 1.38 (q, J = 4.2 Hz, |
| | | | | | | 2H). |
| 457 | 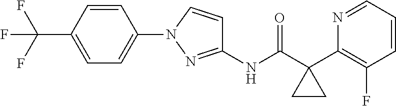 [SMILES: O=C(Nc1ccn(-c2ccc(C(F)(F)F)cc2)n1)C1(c2ncccc2F)CC1] | AB | 12 | >5 | 391.11 | 1H NMR (400 MHz, DMSO-d6) δ 10.27 (s, 1H), 8.55 (d, J = 2.7 Hz, 1H), 8.37 (dt, J = 4.7, 1.6 Hz, 1H), 7.97 (d, J = 8.5 Hz, 2H), 7.84 (d, J = 8.6 Hz, 2H), 7.68 (ddd, J = 10.3, 8.4, 1.4 Hz, 1H), 7.43 (dt, |
| | | | | | | J = 8.4, 4.3 Hz, 1H), 6.83 (d, |
| | | | | | | J = 2.6 Hz, 1H), 1.63 (q, J = |
| | | | | | | 4.2 Hz, 2H), 1.39 (q, J = 4.2 |
| | | | | | | Hz, 2H). |
| 458 | 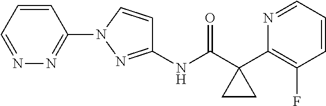 [SMILES: O=C(Nc1ccn(-c2cccnn2)n1)C1(c2ncccc2F)CC1] | AB | 5 | 0.94 | 325.06 | 1H NMR (400 MHz, DMSO-d6) δ 10.37 (s, 1H), 9.16 (dd, J = 4.7, 1.5 Hz, 1H), 8.72 (d, J = 2.8 Hz, 1H), 8.38 (dt, J = 4.7, 1.6 Hz, 1H), 7.98 (dd, J = 8.9, 1.5 Hz, 1H), 7.86 (dd, J = |
| | | | | | | 8.9, 4.7 Hz, 1H), 7.69 (ddd, |
| | | | | | | J = 10.4, 8.4, 1.4 Hz, 1H), |
| | | | | | | 7.43 (dt, J = 8.5, 4.4 Hz, |
| | | | | | | 1H), 6.90 (d, J = 2.8 Hz, |
| | | | | | | 1H), 1.64 (q, J = 4.2 Hz, |
| | | | | | | 2H), 1.41 (q, J = 4.2 Hz, |
| | | | | | | 2H). |
| 459 | 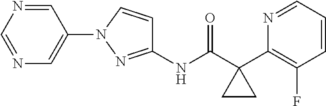 [SMILES: O=C(Nc1ccn(-c2cncnc2)n1)C1(c2ncccc2F)CC1] | AB | 8 | 0.92 | 325.10 | 1H NMR (400 MHz, DMSO-d6) δ 10.36 (s, 1H), 9.21 (s, 2H), 9.08 (s, 1H), 8.57 (d, J = 2.7 Hz, 1H), 8.37 (dt, J = 4.7, 1.6 Hz, 1H), 7.68 (ddd, J = 10.2, 8.4, 1.4 Hz, 1H), 7.43 (dt, |
| | | | | | | J = 8.5, 4.4 Hz, 1H), 6.88 (d, |
| | | | | | | J = 2.7 Hz, 1H), 1.63 (q, J = |
| | | | | | | 4.2 Hz, 2H), 1.40 (q, J = 4.2 |
| | | | | | | Hz, 2H). |
| 460 | 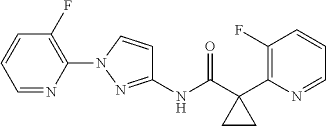 [SMILES: O=C(Nc1ccn(-c2ncccc2F)n1)C1(c2ncccc2F)CC1] | AB | 15 | >2 | 342.06 | 1H NMR (400 MHz, DMSO-d6) δ 10.30 (s, 1H), 8.40-8.29 (m, 3H), 7.97 (ddd, J = 11.5, 8.3, 1.4 Hz, 1H), 7.68 (ddd, J = 10.1, 8.4, 1.4 Hz, 1H), 7.51- 7.37 (m, 2H), 6.83 (d, J = 2.7 Hz, 1H), 1.63 (q, J = 4.2 |
| | | | | | | Hz, 2H), 1.39 (q, J = 4.2 Hz, |
| | | | | | | 2H). |
| 461 | 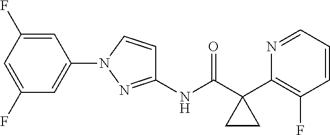 [SMILES: O=C(Nc1ccn(-c2cc(F)cc(F)c2)n1)C1(c2ncccc2F)CC1] | AB | 7 | 0.076 | 359.07 | 1H NMR (400 MHz, DMSO-d6) δ 10.24 (s, 1H), 8.50 (d, J = 2.7 Hz, 1H), 8.37 (dt, J = 4.7, 1.5 Hz, 1H), 7.68 (ddd, J = 10.3, 8.4, 1.4 Hz, 1H), 7.53 (h, J = 5.2 Hz, 2H), 7.43 (dt, J = 8.5, 4.4 Hz, 1H), 7.13 (tt, J = 9.2, 2.3 Hz, 1H), 6.81 (d, |
| | | | | | | J = 2.7 Hz, 1H), 1.62 (q, J = |
| | | | | | | 4.2 Hz, 2H), 1.39 (q, J = 4.2 |
| | | | | | | Hz, 2H). |
| 462 | 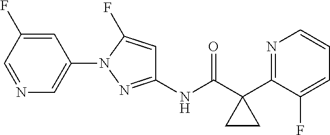 [SMILES: O=C(Nc1cc(F)n(-c2cncc(F)c2)n1)C1(c2ncccc2F)CC1] | AB | 2 | >5 | 360.04 | 1H NMR (400 MHz, DMSO-d6) δ 9.08 (s, 1H), 8.31 (dt, J = 4.7, 1.6 Hz, 1H), 8.14 (s, 1H), 8.01 (s, 1H), 7.68 (ddd, J = 9.9, 8.3, 1.4 Hz, 1H), 7.41 (dt, J = 8.6, 4.4 Hz, 1H), 7.18 (d, J = 11.5 Hz, 1H), 5.83 (d, J = 5.9 Hz, 1H), 1.50 (q, J = 4.0 |
| | | | | | | Hz, 2H), 1.35 (q, J = 4.0 Hz, |
| | | | | | | 2H). |
| 463 | 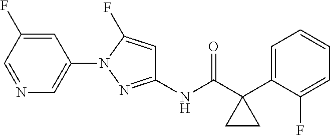 [SMILES: O=C(Nc1cc(F)n(-c2cncc(F)c2)n1)C1(c2ccccc2F)CC1] | AB | 15 | >5 | 358.99 | 1H NMR (400 MHz, DMSO-d6) δ 9.66 (s, 1H), 8.13 (s.1H), 8.02 (s, 1H), 7.51-7.38 (m, 2H), 7.26- 7.08 (m, 2H), 7.07 (ddd, J = 11.2, 7.5, 1.7 Hz, 1H), 5.87 (d, J = 4.6 Hz, 1H), 1.72 (q, J = 4.6 Hz, 2H), 1.48 (q, J = 4.5 Hz, 2H). |
| 464 | 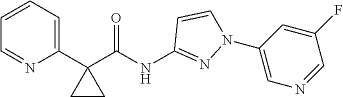 [SMILES: O=C(Nc1ccn(-c2cncc(F)c2)n1)C1(c2ccccn2)CC1] | AB | 56 | 0.13 | 323.88 | 1H NMR (300 MHz, Chloroform-d) δ 11.93 (s, 1H), 8.80-8.75 (m, 1H), 8.72 (ddd, J = 4.9, 1.9, 0.9 Hz, 1H), 8.37 (dd, J = 2.5, 0.5 Hz, 1H), 7.89 (dd, J = |
| | | | | | | 2.7, 0.5 Hz, 1H), 7.86- |
| | | | | | | 7.69 (m, 2H), 7.32-7.23 |
| | | | | | | (m, 3H), 7.15 (dt, J = 8.2, |
| | | | | | | 1.0 Hz, 1H), 7.07 (d, J = 2.6 |
| | | | | | | Hz, 1H), 1.96 (s, 2H), 1.36 |
| | | | | | | (q, J = 4.2 Hz, 2H). |
| 465 | 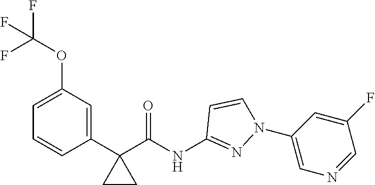 [SMILES: O=C(Nc1ccn(-c2cncc(F)c2)n1)C1(c2cccc(OC(F)(F)F)c2)CC1] | AB | 35 | 0.024 | 406.8 | 1H NMR (300 MHz, Chloroform-d6) δ 8.73- 8.65 (m, 1H), 8.36 (d, J = 2.5 Hz, 1H), 7.86 (dd, J = 2.7, 0.5 Hz, 1H), 7.68 (dt, J = 9.5, 2.4 Hz, 1H), 7.62 (s, 1H), 7.56-7.45 (m, 2H), 7.37 (ddd, J = 2.5, 1.8, 0.9 Hz, 1H), 7.03 (d, J = 2.7 Hz, 1H), 1.80 (q, J = 3.9 Hz, 2H), 1.24 (q, J = 4.0 Hz, 2H). |
| 466 | 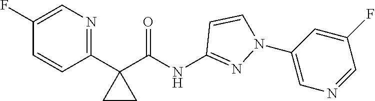 [SMILES: O=C(Nc1ccn(-c2cncc(F)c2)n1)C1(c2ccc(F)cn2)CC1] | AB | 40 | 0.56 | 341.85 | 1H NMR (300 MHz, Chloroform-d6) δ 10.91 (s, 1H), 8.75 (dd, J = 2.3, 1.1 Hz, 1H), 8.58 (d, J = 3.0 Hz, 1H), 8.37 (dd, J = 2.5, 0.5 Hz, 1H), 7.88 (dd, J = 2.7, 0.5 Hz, 1H), 7.79 (dt, J = |
| | | | | | | 9.6, 2.3 Hz, 1H), 7.47 (ddd, |
| | | | | | | J = 8.8, 7.9, 3.0 Hz, 1H), |
| | | | | | | 7.23 (ddd, J = 8.8, 4.2, 0.7 |
| | | | | | | Hz, 1H), 7.05 (d, J = 2.6 Hz, |
| | | | | | | 1H), 2.00-1.80 (m, 2H), |
| | | | | | | 1.44-1.17 (m, 2H). |
| 467 | 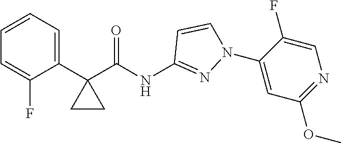 [SMILES: COc1cc(-n2ccc(NC(=O)C3(c4ccccc4F)CC3)n2)c(F)cn1] | AE | 3 | 2.7 | 371.01 | 1H NMR (400 MHz, DMSO-d6) δ 9.71 (s, 1H), 8.32 (m, 1H), 8.24 (m, 1H), 7.52-7.36 (m, 2H), 7.25- 7.15 (m, 2H), 7.13 (d, J = 5.5 Hz, 1H), 6.88 (d, J = 2.8 Hz, 1H), 3.85 (s, 3H), 1.61 (m, 2H), 1.17 (m, 2H). |
| 468 | 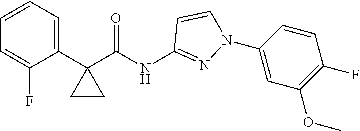 [SMILES: COc1cc(-n2ccc(NC(=O)C3(c4ccccc4F)CC3)n2)ccc1F] | AE | 10 | >5 | 370.08 | 1H NMR (400 MHz, DMSO-d6) δ 9.41 (s, 1H), 8.39 (d, J = 2.6 Hz, 1H), 7.48 (m, 2H), 7.45-7.36 (m, 1H), 7.34-7.18 (m, 5H), 6.73 (d, J = 2.6 Hz, 1H), 3.87 (s, 3H), 1.60 (m, 2H), 1.15 (m, 2H). |
| 469 | 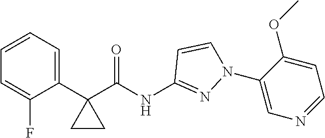 [SMILES: COc1ccncc1-n1ccc(NC(=O)C2(c3ccccc3F)CC2)n1] | AE | 9 | >2 | 353.05 | 1H NMR (400 MHz, DMSO-d6) δ 9.43 (s, 1H), 8.17 (d, J = 2.6 Hz, 1H), 7.57-7.35 (m, 3H), 7.21 (m, 2H), 6.78 (d, J = 2.6 Hz, 1H), 4.05 (s, 3H), 1.60 (m, 2H), 1.16 (m, 2H). |
| 470 | 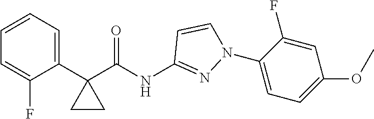 [SMILES: COc1ccc(-n2ccc(NC(=O)C3(c4ccccc4F)CC3)n2)c(F)c1] | AE | 6 | >2 | 370.04 | 1H NMR (400 MHz, DMSO-d6) δ 9.31 (s, 1H), 7.94 (t, J = 2.6 Hz, 1H), 7.60-7.34 (m, 3H), 7.26- 7.16 (m, 2H), 7.06 (m, 1H), 6.87 (m, 1H), 6.70 (d, J = 2.6 Hz, 1H), 3.80 (s, 3H), 1.59 (m, 2H), 1.14 (m, 2H). |
| 471 | 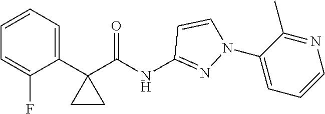 [SMILES: Cc1ncccc1-n1ccc(NC(=O)C2(c3ccccc3F)CC2)n1] | AE | 21 | >5 | 337.29 | 1H NMR (400 MHz, DMSO-d6) δ 9.30 (s, 1H), 8.50 (m, 1H), 7.99 (d, J = 2.5 Hz, 1H), 7.75 (m, 1H), 7.53-7.44 (m, 1H), 7.44- 7.35 (m, 2H), 7.29-7.15 (m, 2H), 6.73 (d, J = 2.5 Hz, 1H), 2.42 (s, 3H), 1.59 |
| | | | | | | (m, 2H), 1.14 (m, 2H). |
| 472 | 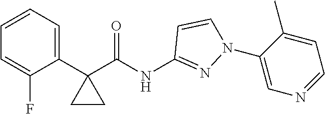 [SMILES: Cc1ccncc1-n1ccc(NC(=O)C2(c3ccccc3F)CC2)n1] | AE | 10 | 0.056 | 337.29 | 1H NMR (400 MHz, DMSO-d6) δ 9.33 (s, 1H), 8.49 (s, 1H), 8.45 (d, J = 5.0 Hz, 1H), 8.01 (d, J = 2.5 Hz, 1H), 7.51-7.34 (m, 3H), 7.28-7.13 (m, 2H), 6.74 (d, J = 2.5 Hz, 1H), 2.27 (s, 3H), 1.59 (m, 2H), |
| | | | | | | 1.14 (m, 2H). |
| 473 | 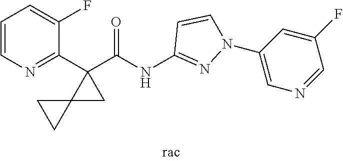 [SMILES: O=C(Nc1ccn(-c2cncc(F)c2)n1)C1(c2ncccc2F)CC12CC2] | AD | 39 | 0.43 | 368.08 | 1H NMR (400 MHz, Chloroform-d) δ 8.64 (dd, J = 2.2, 1.0 Hz, 1H), 8.51 (s, 1H), 8.42 (dt, J = 4.6, 1.5 Hz, 1H), 8.28 (d, J = 2.5 Hz, 1H), 7.77 (d, J = 2.7 Hz, 1H), 7.64 (dt, J = 9.5, 2.4 Hz, 1H), 7.39 (ddd, J = 9.7, 8.3, 1.4 Hz, 1H), 7.25 (ddd, J = 8.4, 4.7, 4.0 Hz, 1H), 6.98 (d, J = 2.7 Hz, 1H), |
| | | | | | | 2.22 (d, J = 4.8 Hz, 1H), |
| | | | | | | 2.15-2.08 (m, 1H), 1.24- |
| | | | | | | 1.11 (m, 2H), 0.97 (dt, J = |
| | | | | | | 9.9, 5.2 Hz, 1H), 0.77 (dt, |
| | | | | | | J = 8.9, 5.3 Hz, 1H). |
| 474 | 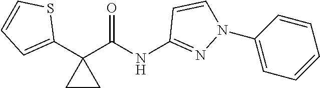 [SMILES: O=C(Nc1ccn(-c2ccccc2)n1)C1(c2cccs2)CC1] | AC | 43 | 0.019 | 310.04 | 1H NMR (300 MHz, Chloroform-d) δ 8.12 (s, 1H), 7.81 (dd, J = 2.6, 0.5 Hz, 1H), 7.62-7.52 (m, 2H), 7.48-7.38 (m, 2H), 7.35 (dd, J = 5.2, 1.2 Hz, |
| | | | | | | 1H), 7.28-7.17 (m, 2H), |
| | | | | | | 7.07 (dd, J = 5.2, 3.5 Hz, |
| | | | | | | 1H), 6.96 (d, J = 2.5 Hz, |
| | | | | | | 1H), 1.84 (q, J = 3.8 Hz, |
| | | | | | | 2H), 1.33 (q, J = 3.8 Hz, |
| | | | | | | 2H). |
| 475 | 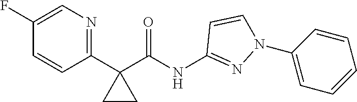 [SMILES: O=C(Nc1ccn(-c2ccccc2)n1)C1(c2ccc(F)cn2)CC1] | AC | 26 | 0.27 | 323.11 | 1H NMR (300 MHz, Chloroform-d) δ 10.39 (s, 1H), 8.57 (d, J = 2.9 Hz, 1H), 7.83 (dd, J = 2.6, 0.6 Hz, 1H), 7.68-7.57 (m, 2H), 7.50-7.39 (m, 3H), 7.29-7.24 (m, 2H), 6.96 |
| | | | | | | (d, J = 2.5 Hz, 1H), 1.94- |
| | | | | | | 1.85 (m, 2H), 1.37-1.24 |
| | | | | | | (m, 2H). |
| 476 | 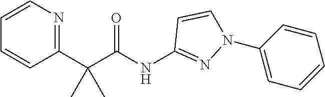 [SMILES: CC(C)(C(=O)Nc1ccn(-c2ccccc2)n1)c1ccccn1] | AC | 19 | 0.53 | 307.13 | 1H NMR (300 MHz, Chloroform-d) δ 9.74 (s, 1H), 8.63 (ddd, J = 4.8, 1.9, 0.9 Hz, 1H), 7.72 (dd, J = 2.6, 0.5 Hz, 1H), 7.67- 7.58 (m, 1H), 7.56-7.49 (m, 2H), 7.43-7.31 (m, |
| | | | | | | 3H), 7.15 (ddd, J = 7.6, 4.8, |
| | | | | | | 1.0 Hz, 2H), 6.88 (d, J = 2.5 |
| | | | | | | Hz, 1H), 1.68 (s, 6H). |
| 477 | 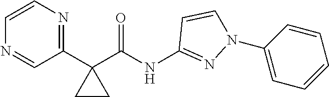 [SMILES: O=C(Nc1ccn(-c2ccccc2)n1)C1(c2cnccn2)CC1] | AC | 34 | 1.5 | 306.09 | 1H NMR (300 MHz, Chloroform-d) δ 10.55 (s, 1H), 8.55 (dd, J = 2.6, 15 Hz, 1H), 8.46 (dd, J = 5.4, 2.0 Hz, 2H), 7.79-7.71 (m, 1H), 7.60-7.49 (m, 2H), 7.43-7.29 (m, 2H), 7.25- |
| | | | | | | 7.12 (m, 1H), 6.88 (d, J = |
| | | | | | | 2.5 Hz, 1H), 1.97-1.87 (m, |
| | | | | | | 2H), 1.41-1.31 (m, 2H). |
| 478 | 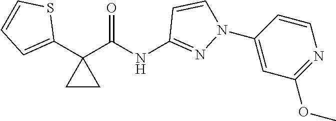 [SMILES: COc1cc(-n2ccc(NC(=O)C3(c4cccs4)CC3)n2)ccn1] | AC | 49 | 0.12 | 341.07 | 1H NMR (300 MHz, Chloroform-d) δ 8.20- 8.08 (m, 2H), 7.87 (dd, J = 2.7, 0.5 Hz, 1H), 7.37 (dd, J = 5.2, 1.2 Hz, 1H), 7.20 (dd, J = 3.5, 1.2 Hz, 1H), 7.16-7.04 (m, 2H), 7.02 (d, J = 2.7 Hz, 1H), 6.91 |
| | | | | | | (dd, J = 2.0, 0.6 Hz, 1H), |
| | | | | | | 3.97 (s, 3H), 1.83 (q, J = |
| | | | | | | 3.8 Hz, 2H), 1.35 (q, J = 3.8 |
| | | | | | | Hz, 2H). |
| 479 | 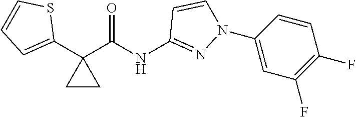 [SMILES: O=C(Nc1ccn(-c2ccc(F)c(F)c2)n1)C1(c2cccs2)CC1] | AC | 42 | 0.19 | 346.04 | 1H NMR (300 MHz, Chloroform-d) δ 7.99 (s, 1H), 7.64 (d, J = 2.6 Hz, 1H), 7.36 (ddd, J = 11.3, 6.9, 2.5 Hz, 1H), 7.27 (dd, J = 5.2, 1.2 Hz, 1H), 7.24- 7.04 (m, 3H), 6.99 (dd, J = 5.2, 3.5 Hz, 1H), 6.88 (d, |
| | | | | | | J = 2.6 Hz, 1H), 1.74 (q, J = |
| | | | | | | 3.8 Hz, 2H), 1.25 (q, J = 3.8 |
| | | | | | | Hz, 2H). |
| 480 | 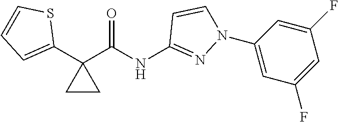 [SMILES: O=C(Nc1ccn(-c2cc(F)cc(F)c2)n1)C1(c2cccs2)CC1] | AC | 32 | 0.23 | 346.00 | 1H NMR (300 MHz, Chloroform-d) δ 8.00 (s, 1H), 7.68 (dd, J = 2.7, 0.6 Hz, 1H), 7.28 (dd, J = 5.2, 1.2 Hz, 1H), 7.11 (dd, J = 3.5, 1.2 Hz, 1H), 7.12- 6.92 (m, 3H), 6.91 (d, J = 2.7 Hz, 1H), 6.59 (tt, J = |
| | | | | | | 8.8, 2.3 Hz, 1H), 1.74 (q, |
| | | | | | | J = 3.8 Hz, 2H), 1.25 (q, J = |
| | | | | | | 3.8 Hz, 2H). |
| 481 | 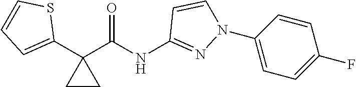 [SMILES: O=C(Nc1ccn(-c2ccc(F)cc2)n1)C1(c2cccs2)CC1] | AC | 49 | 0.16 | 328.08 | 1H NMR (300 MHz, Chloroform-d) δ 8.09 (s, 1H), 7.73 (dd, J = 2.6, 0.6 Hz, 1H), 7.59-7.45 (m, 2H), 7.35 (dd, J = 5.2, 1.2 Hz, 1H), 7.19 (dd, J = 3.5, |
| | | | | | | 1.2 Hz, 1H), 7.19-7.02 (m, |
| | | | | | | 3H), 6.95 (d, J = 2.5 Hz, |
| | | | | | | 1H), 1.83 (q, J = 3.8 Hz, |
| | | | | | | 2H), 1.33 (q, J = 3.8 Hz, |
| | | | | | | 2H). |
| 482 | 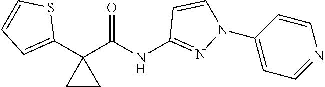 [SMILES: O=C(Nc1ccn(-c2ccncc2)n1)C1(c2cccs2)CC1] | AC | 52 | 0.039 | 311.05 | 1H NMR (300 MHz, Chloroform-d) δ 8.65- 8.56 (m, 2H), 8.13 (s, 1H), 7.92 (dd, J = 2.7, 0.6 Hz, 1H), 7.54-7.44 (m, 2H), 7.38 (dd, J = 5.2, 1.2 Hz, |
| | | | | | | 1H), 7.21 (dd, J = 3.5, 1.2 |
| | | | | | | Hz, 1H), 7.14-7.01 (m, |
| | | | | | | 2H), 1.84 (q, J = 3.8 Hz, |
| | | | | | | 2H), 1.35 (q, J = 3.8 Hz, |
| | | | | | | 2H). |
| 483 | 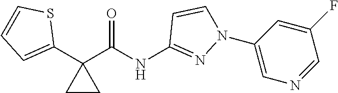 [SMILES: O=C(Nc1ccn(-c2cncc(F)c2)n1)C1(c2cccs2)CC1] | AC | 47 | <0.02 | 329.05 | 1H NMR (300 MHz, Chloroform-d) δ 8.74- 8.66 (m, 1H), 8.36 (dd, J = 2.5, 0.5 Hz, 1H), 8.11 (s, 1H), 7.86 (dd, J = 2.7, 0.6 Hz, 1H), 7.70 (dt, J = 9.5, |
| | | | | | | 2.4 Hz, 1H), 7.38 (dd, J = |
| | | | | | | 5.2, 1.2 Hz, 1H), 7.21 (dd, |
| | | | | | | J = 3.5, 1.2 Hz, 1H), 7.14- |
| | | | | | | 7.01 (m, 2H), 1.84 (q, J = |
| | | | | | | 3.8 Hz, 2H), 1.35 (q, J = 3.8 |
| | | | | | | Hz, 2H). |
| 484 | 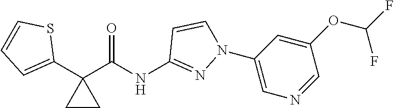 [SMILES: O=C(Nc1ccn(-c2cncc(OC(F)F)c2)n1)C1(c2cccs2)CC1] | AC | 31 | >5 | 377.06 | 1H NMR (300 MHz, Chloroform-d) δ 8.74 (dd, J = 2.3, 0.5 Hz, 1H), 8.41- 8.33 (m, 1H), 8.12 (s, 1H), 7.87 (dd, J = 2.7, 0.6 Hz, 1H), 7.80-7.71 (m, 1H), 7.38 (dd, J = 5.2, 1.2 Hz, |
| | | | | | | 1H), 7.22 (dd, J = 3.5, 1.2 |
| | | | | | | Hz, 1H), 7.15-7.01 (m, |
| | | | | | | 2H), 6.61 (t, J = 72.4 Hz, |
| | | | | | | 1H), 1.84 (q, J = 3.8 Hz, |
| | | | | | | 2H), 1.36 (q, J = 3.8 Hz, |
| | | | | | | 2H). |
| 485 | 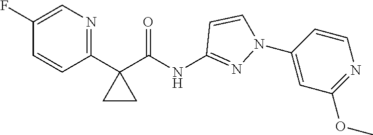 [SMILES: COc1cc(-n2ccc(NC(=O)C3(c4ccc(F)cn4)CC3)n2)ccn1] | AC | 36 | 1.5 | 354.10 | 1H NMR (300 MHz, Chloroform-d) δ 10.79 (s, 1H), 8.58 (d, J = 2.9 Hz, 1H), 8.27-8.13 (m, 1H), 7.89 (d, J = 2.7 Hz, 1H), 7.46 (ddd, J = 8.8, 7.9, 3.0 Hz, 1H), 7.29-7.13 (m, 2H), 7.09-6.95 (m, 2H), 3.99 (s, 3H), 1.91 (q, J = |
| | | | | | | 4.2 Hz, 2H), 1.41-1.21 (m, |
| | | | | | | 2H). |
| 486 | 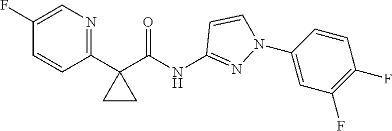 [SMILES: O=C(Nc1ccn(-c2ccc(F)c(F)c2)n1)C1(c2ccc(F)cn2)CC1] | AC | 36 | RND | 359.06 | 1H NMR (300 MHz, Chloroform-d) δ 10.57 (s, 1H), 8.48 (d, J = 3.0 Hz, 1H), 7.66 (dd, J = 2.6, 0.5 Hz, 1H), 7.50-7.30 (m, 2H), 7.30-7.06 (m, 3H), 6.88 (d, J = 2.6 Hz, 1H), 1.87-1.71 (m, 2H), 1.21 (p, J = 4.1 Hz, 2H). |
| 487 | 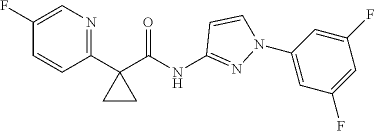 [SMILES: O=C(Nc1ccn(-c2cc(F)cc(F)c2)n1)C1(c2ccc(F)cn2)CC1] | AC | 24 | 1.2 | 359.06 | 1H NMR (300 MHz, Chloroform-d) δ 10.67 (s, 1H), 8.49 (d, J = 3.0 Hz, 1H), 7.70 (dd, J = 2.7, 0.6 Hz, 1H), 7.38 (ddd, J = 8.8, 7.9, 3.0 Hz, 1H), 7.20- 7.04 (m, 3H), 6.91 (d, J = 2.6 Hz, 1H), 6.60 (tt, J = 8.7, 2.3 Hz, 1H), 1.87- |
| | | | | | | 1.72 (m, 2H), 1.33-1.12 |
| | | | | | | (m, 2H). |
| 488 | 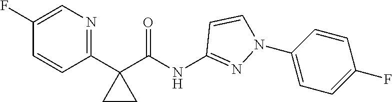 [SMILES: O=C(Nc1ccn(-c2ccc(F)cc2)n1)C1(c2ccc(F)cn2)CC1] | AC | 40 | 0.51 | 341.10 | 1H NMR (300 MHz, Chloroform-d) δ 10.48 (s, 1H), 8.56 (d, J = 3.0 Hz, 1H), 7.75 (d, J = 2.5 Hz, 1H), 7.65-7.53 (m, 2H), 7.45 (ddd, J = 8.8, 7.9, 3.0 Hz, 1H), 7.31-7.20 (m, |
| | | | | | | 3H), 7.21-7.06 (m, 2H), |
| | | | | | | 6.95 (d, J = 2.5 Hz, 1H), |
| | | | | | | 1.90 (q, J = 4.2 Hz, 2H), |
| | | | | | | 1.35-1.24 (m, 2H). |
| 489 | 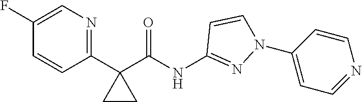 [SMILES: O=C(Nc1ccn(-c2ccncc2)n1)C1(c2ccc(F)cn2)CC1] | AC | 30 | 1.5 | 324.08 | 1H NMR (400 MHz, Chloroform-d) δ 10.74 (s, 1H), 8.58-8.46 (m, 3H), 7.85 (dd, J = 2.7, 0.6 Hz, 1H), 7.51-7.44 (m, 2H), 7.38 (ddd, J = 8.8, 7.9, 3.0 Hz, 1H), 7.15 (ddd, J = 8.9, |
| | | | | | | 4.2, 0.6 Hz, 1H), 6.97 (d, |
| | | | | | | J = 2.7 Hz, 1H), 1.86-1.79 |
| | | | | | | (m, 2H), 1.27-1.15 (m, |
| | | | | | | 2H). |
| 490 | 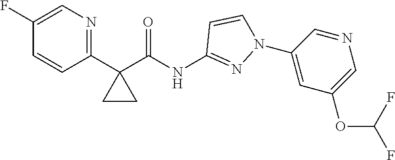 [SMILES: O=C(Nc1ccn(-c2cncc(OC(F)F)c2)n1)C1(c2ccc(F)cn2)CC1] | AC | 13 | >5 | 390.09 | 1H NMR (400 MHz, Chloroform-d) δ 10.75 (s, 1H), 8.71 (d, J = 2.3 Hz, 1H), 8.50 (d, J = 2.9 Hz, 1H), 8.32-8.26 (m, 1H), 7.82-7.71 (m, 2H), 7.38 (ddd, J = 8.8, 7.9, 3.0 Hz, 1H), 7.15 (ddd, J = 8.8, 4.2, 0.6 Hz, 1H), 6.96 (d, J = 2.6 Hz, 1H), 6.55 (t, J = 72.4 |
| | | | | | | Hz, 1H), 1.87-1.79 (m, |
| | | | | | | 2H), 1.27-1.15 (m, 2H). |
| 491 | 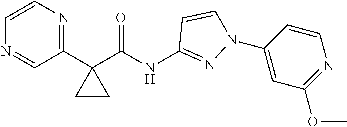 [SMILES: COc1cc(-n2ccc(NC(=O)C3(c4cnccn4)CC3)n2)ccn1] | AC | 30 | >5 | 337.11 | 1H NMR (400 MHz, Chloroform-d) δ 10.93 (s, 1H), 8.66 (dd, J = 2.7, 1.5 Hz, 1H), 8.56 (dd, J = 21.8, 2.0 Hz, 2H), 8.19 (d, J = 5.7 Hz, 1H), 7.90 (d, J = 2.7 Hz, 1H), 7.19 (dd, J = 5.8, 1.9 Hz, 1H), 7.02 (dd, J = 12.2, |
| | | | | | | 2.3 Hz, 2H), 4.00 (d, J = 1.0 |
| | | | | | | Hz, 3H), 2.03 (q, J = 4.5 Hz, |
| | | | | | | 2H), 1.47 (q, J = 4.5 Hz, |
| | | | | | | 2H). |
| 492 | 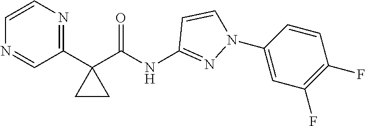 [SMILES: O=C(Nc1ccn(-c2ccc(F)c(F)c2)n1)C1(c2cnccn2)CC1] | AC | 47 | >5 | 342.11 | 1H NMR (400 MHz, Chloroform-d) δ 10.81 (s, 1H), 8.65 (dd, J = 2.6, 1.5 Hz, 1H), 8.56 (dd, J = 16.5, 2.0 Hz, 2H), 7.77 (d, J = 2.6 Hz, 1H), 7.54 (ddd, J = 11.5, 6.9, 2.6 Hz, 1H), 7.34 (dt, J = 9.7, 2.4 Hz, 1H), |
| | | | | | | 7.24 (q, J = 9.1 Hz, 1H), |
| | | | | | | 6.99 (d, J = 2.6 Hz, 1H), |
| | | | | | | 2.02 (q, J = 4.4 Hz, 2H), |
| | | | | | | 1.47 (q, J = 4.5 Hz, 2H). |
| 493 | 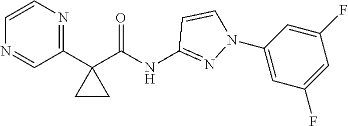 [SMILES: O=C(Nc1ccn(-c2cc(F)cc(F)c2)n1)C1(c2cnccn2)CC1] | AC | 16 | >5 | 342.08 | 1H NMR (400 MHz, Chloroform-d) δ 10.90 (s, 1H), 8.66 (dd, J = 2.6, 1.6 Hz, 1H), 8.61-8.50 (m, 2H), 7.80 (dd, J = 2.7, 0.6 Hz, 1H), 7.31-7.15 (m, 2H), 7.01 (d, J = 2.7 Hz, 1H), 6.70 (tt, J = 8.8, 2.3 |
| | | | | | | Hz, 1H), 2.08-1.94 (m, |
| | | | | | | 2H), 1.54-1.43 (m, 2H). |
| 494 | 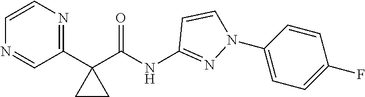 [SMILES: O=C(Nc1ccn(-c2ccc(F)cc2)n1)C1(c2cnccn2)CC1] | AC | 53 | >5 | 324.12 | 1H NMR (400 MHz, Chloroform-d) δ 10.66 (s, 1H), 8.64 (dd, J = 2.6, 1.5 Hz, 1H), 8.55 (dd, J = 8.1, 2.1 Hz, 2H), 7.77 (dd, J = 2.6, 0.5 Hz, 1H), 7.65- 7.54 (m, 2H), 7.20-7.09 |
| | | | | | | (m, 2H), 6.96 (d, J = 2.5 |
| | | | | | | Hz, 1H), 2.05-1.97 (m, |
| | | | | | | 2H), 1.50-1.42 (m, 2H). |
| 495 | 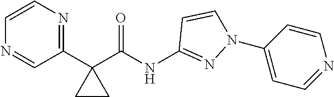 [SMILES: O=C(Nc1ccn(-c2ccncc2)n1)C1(c2cnccn2)CC1] | AC | 40 | >5 | 307.09 | 1H NMR (400 MHz, Chloroform-d) δ 10.89 (s, 1H), 8.60-8.52 (m, 3H), 8.49 (d, J = 2.6 Hz, 1H), 8.44 (d, J = 1.6 Hz, 1H), 7.87 (dd, J = 2.7, 0.5 Hz, 1H), 7.51-7.44 (m, 2H), |
| | | | | | | 6.98 (d, J = 2.7 Hz, 1H), |
| | | | | | | 1.98-1.90 (m, 2H), 1.43- |
| | | | | | | 1.35 (m, 2H). |
| 496 | 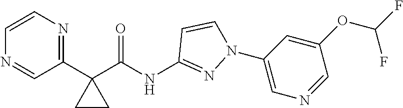 [SMILES: O=C(Nc1ccn(-c2cncc(OC(F)F)c2)n1)C1(c2cnccn2)CC1] | AC | 22 | >5 | 373.07 | 1H NMR (400 MHz, Chloroform-d) δ 10.90 (s, 1H), 8.72 (t, J = 1.9 Hz, 1H), 8.58 (q, J = 1.9 Hz, 1H), 8.46 (dt, J = 21.9, 2.0 Hz, 2H), 8.30 (d, J = 2.4 Hz, 1H), 7.78 (dt, J = 22.3, 2.4 |
| | | | | | | Hz, 2H), 6.97 (t, J = 2.2 Hz, |
| | | | | | | 1H), 6.55 (td, J = 72.4, 1.7 |
| | | | | | | Hz, 1H), 1.95 (q, J = 4.4 Hz, |
| | | | | | | 2H), 1.39 (q, J = 4.5 Hz, |
| | | | | | | 2H). |
| 497 | 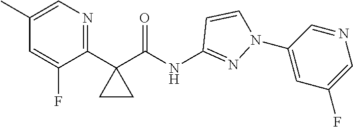 [SMILES: Cc1cnc(C2(C(=O)Nc3ccn(-c4cncc(F)c4)n3)CC2)c(F)c1] | AD | 28 | 2.1 | 356.11 | 1H NMR (400 MHz, Chloroform-d) δ 8.65- 8.59 (m, 1H), 8.27 (td, J = 2.6, 0.9 Hz, 3H), 7.76 (dd, J = 2.7, 0.5 Hz, 1H), 7.60 (dt, J = 9.5, 2.3 Hz, 1H), 7.22 (ddd, J = 10.5, 1.8, 0.9 Hz, 1H), 6.96 (d, J = 2.7 Hz, 1H), 2.35 (d, J = 0.8 Hz, |
| | | | | | | 3H), 1.74 (qd, J = 4.3, 0.8 |
| | | | | | | Hz, 2H), 1.31 (qd, J = 4.3, |
| | | | | | | 0.7 Hz, 2H). |
| 498 | 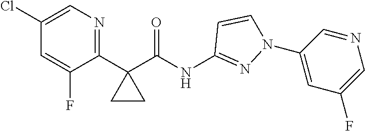 [SMILES: O=C(Nc1ccn(-c2cncc(F)c2)n1)C1(c2ncc(Cl)cc2F)CC1] | AD | 45 | 0.47 | 376.05 | 1H NMR (400 MHz, Chloroform-d) δ 8.65- 8.59 (m, 1H), 8.42 (dd, J = 2.1, 0.7 Hz, 1H), 8.28 (dd, J = 2.6, 0.5 Hz, 1H), 8.09 (s, 1H), 7.77 (dd, J = 2.7, 0.5 Hz, 1H), 7.61 (dt, J = 9.5, 2.4 Hz, 1H), 7.46 (dd, J = 9.1, 2.1 Hz, 1H), 6.95 (d, |
| | | | | | | J = 2.6 Hz, 1H), 1.76 (qd, |
| | | | | | | J = 4.4, 0.8 Hz, 2H), 1.41- |
| | | | | | | 1.29 (m, 2H). |
| 501 | 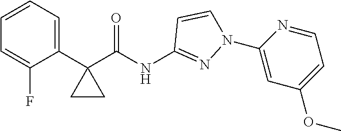 [SMILES: COc1ccnc(-n2ccc(NC(=O)C3(c4ccccc4F)CC3)n2)c1] | AE | 6 | >5 | 353.09 | 1H NMR (400 MHz, DMSO-d6) δ 9.54 (s, 1H), 8.47 (d, J = 2.7 Hz, 1H), 8.23 (d, J = 5.8 Hz, 1H), 7.53-7.34 (m, 2H), 7.27- 7.14 (m, 3H), 6.87 (dd, J = 5.7, 2.4 Hz, 1H), 6.78 (d, J = 2.7 Hz, 1H), 3.86 (s, 3H), 1.61 (m, 2H), 1.16 (m, 2H). |
| 502 | 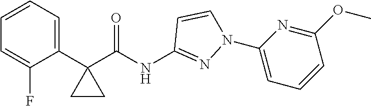 [SMILES: COc1cccc(-n2ccc(NC(=O)C3(c4ccccc4F)CC3)n2)n1] | AE | 3 | >5 | 353.09 | 1H NMR (400 MHz, DMSO-d6) δ .48 (s, 1H), 8.50 (d, J = 2.7 Hz, 1H), 7.80 (m, 1H), 7.53-7.37 (m, 2H), 7.30-7.15 (m, 3H), 6.78 (d, J = 2.7 Hz, 1H), 6.69 (d, J = 8.1 Hz, |
| | | | | | | 1H), 3.92 (s, 3H), 1.61 (m, |
| | | | | | | 2H), 1.16 (m, 2H). |
| 503 | 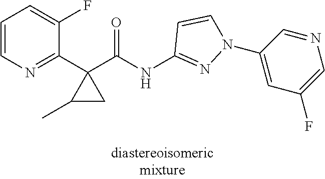 [SMILES: CC1CC1(C(=O)Nc1ccn(-c2cncc(F)c2)n1)c1ncccc1F] | AD | 11 | 0.11 | 356.15 | 1H NMR (400 MHz, Chloroform-d) δ 8.75- 8.69 (m, 1H), 8.49 (dt, J = 4.7, 1.3 Hz, 1H), 8.33 (dd, J = 2.6, 1.0 Hz, 1H), 7.89 (dd, J = 2.7, 1.0 Hz, 1H), 7.76 (dtd, J = 9.4, 2.4, 0.9 Hz, 1H), 7.53 (ddd, J = 9.7, 8.3, 1.4 Hz, 1H), 7.40 (dt, J = 8.5, 4.4 Hz, 1H), 7.04 (dd, J = 2.7, 1.0 Hz, 1H), 1.95 (ddd, J = 9.1, 7.8, 6.4 |
| | | | | | | Hz, 1H), 1.86-1.79 (m, |
| | | | | | | 1H), 1.40 (ddt, J = 9.0, 5.2, |
| | | | | | | 1.1 Hz, 1H), 1.33 (dd, J = |
| | | | | | | 6.2, 1.0 Hz, 3H). |
| 504 | 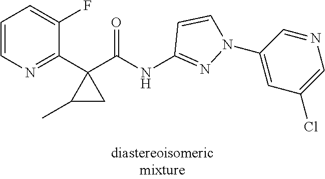 [SMILES: CC1CC1(C(=O)Nc1ccn(-c2cncc(Cl)c2)n1)c1ncccc1F] | AD | 23 | 0.12 | 372.09 | 1H NMR (400 MHz, Chloroform-d) δ 8.76 (d, J = 2.3 Hz, 1H), 8.50 (dt, J = 4.8, 1.3 Hz, 1H), 8.42 (d, J = 2.1 Hz, 1H), 7.99 (t, J = 2.2 Hz, 1H), 7.86 (d, J = 2.7 Hz, 1H), 7.51 (ddd, J = 9.7, 8.3, 1.4 Hz, 1H), 7.38 (ddd, J = 8.5, 4.7, 4.0 Hz, 1H), 7.05 (d, J = 2.7 Hz, 1H), 1.93 (ddd, J = 8.8, 7.2, 6.1 |
| | | | | | | Hz, 1H), 1.83 (dd, J = 7.4, |
| | | | | | | 5.1 Hz, 1H), 1.40 (ddd, J = |
| | | | | | | 8.8, 5.1, 1.2 Hz, 1H), 1.33 |
| | | | | | | (d, J = 6.2 Hz, 3H). |
| 505 | 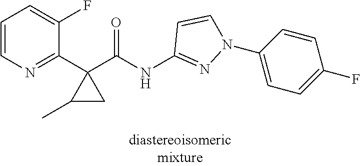 [SMILES: CC1CC1(C(=O)Nc1ccn(-c2ccc(F)cc2)n1)c1ncccc1F] | AD | 33 | 0.11 | 355.14 | 1H NMR (400 MHz, Methanol-d4) δ 8.45 (dt, J = 4.9, 1.2 Hz, 1H), 8.08 (d, J = 2.6 Hz, 1H), 7.76 (ddd, J = 10.0, 8.4, 1.3 Hz, 1H), 7.73-7.64 (m, 2H), 7.53 (dt, J = 8.7, 4.5 Hz, 1H), 7.24-7.08 (m, 2H), 6.81 (d, J = 2.6 Hz, 1H), 2.25 (dt, J = 8.8, 6.6 Hz, 1H), 1.77 (dd, J = 7.2, 5.0 Hz, |
| | | | | | | 1H), 1.35-1.21 (m, 5H). |
| 506 | 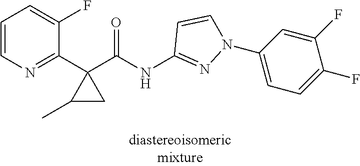 [SMILES: CC1CC1(C(=O)Nc1ccn(-c2ccc(F)c(F)c2)n1)c1ncccc1F] | AD | 38 | 0.22 | 373.14 | 1H NMR (400 MHz, Methanol-d4) δ 8.41 (dt, J = 4.9, 1.4 Hz, 1H), 8.12 (d, J = 2.7 Hz, 1H), 7.76-7.57 (m, 2H), 7.57-7.47 (m, 1H), 7.43 (dt, J = 8.6, 4.4 Hz, 1H), 7.33 (dt, J = 10.2, 8.7 Hz, 1H), 6.84 (d, J = 2.7 Hz, 1H), 2.30 (dt, J = 8.8, 6.6 Hz, 1H), 1.72 (dd, J = 7.2, 4.9 Hz, 1H), 1.28 (d, |
| | | | | | | J = 6.3 Hz, 4H), 1.20 (dd, |
| | | | | | | J = 9.0, 4.8 Hz, 1H). |
| 507 | 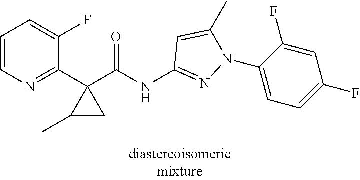 [SMILES: Cc1cc(NC(=O)C2(c3ncccc3F)CC2C)nn1-c1ccc(F)cc1F] | AD | 33 | >5 | 387.19 | 1H NMR (400 MHz, Methanol-d4) δ 8.45 (dt, J = 4.9, 1.2 Hz, 1H), 7.80 (ddd, J = 9.9, 8.4, 1.3 Hz, 1H), 7.61-7.43 (m, 2H), 7.22 (ddd, J = 10.3, 8.7, 2.7 Hz, 1H), 7.12 (dddd, J = 8.8, 8.0, 2.8, 1.5 Hz, 1H), 6.57 (s, 1H), 2.21 (dt, J = 9.0, 6.8 Hz, 1H), 2.18- 2.13 (m, 3H), 1.78 (dd, J = 7.2, 5.1 Hz, 1H), 1.36- |
| | | | | | | 1.23 (m, 4H). |
| 508 | 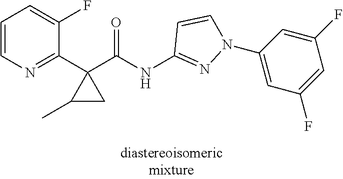 [SMILES: CC1CC1(C(=O)Nc1ccn(-c2cc(F)cc(F)c2)n1)c1ncccc1F] | AD | 27 | 0.071 | 373.14 | 1H NMR (400 MHz, Methanol-d4) δ 8.41 (dt, J = 4.9, 1.4 Hz, 1H), 8.21 (d, J = 2.8 Hz, 1H), 7.71-7.56 (m, 1H), 7.50-7.33 (m, 3H), 6.93-6.74 (m, 2H), 2.32 (dt, J = 8.8, 6.7 Hz, 1H), 1.71 (dd, J = 7.2, 4.8 Hz, 1H), 1.30-1.26 (m, 3H), 1.19 (dd, J = 8.9, 4.8 Hz, 1H). |
| 509 | 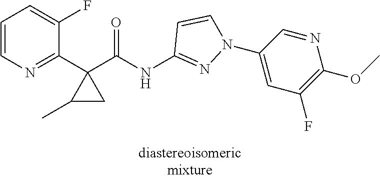 [SMILES: COc1ncc(-n2ccc(NC(=O)C3(c4ncccc4F)CC3C)n2)cc1F] | AD | 15 | 0.18 | 386.1 | 1H NMR (400 MHz, Methanol-d4) δ 8.50 (d, J = 5.0 Hz, 1H), 8.32 (d, J = 2.3 Hz, 1H), 8.11 (d, J = 2.7 Hz, 1H), 8.03-7.82 (m, 2H), 7.63 (dt, J = 8.9, 4.6 Hz, 1H), 6.83 (d, J = 2.6 Hz, 1H), 4.01 (s, 3H), 1.83 (dd, J = 7.2, 5.1 Hz, 1H), 1.38- 1.26 (m, 4H). |
| 510 | 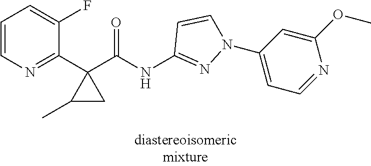 [SMILES: COc1cc(-n2ccc(NC(=O)C3(c4ncccc4F)CC3C)n2)ccn1] | AD | 45 | 0.67 | 368.13 | 1H NMR (400 MHz, Methanol-d4) δ 8.46 (d, J = 2.9 Hz, 1H), 8.40 (dt, J = 4.7, 1.4 Hz, 1H), 8.21 (dd, J = 6.4, 0.5 Hz, 1H), 7.65- 7.55 (m, 2H), 7.49-7.44 (m, 1H), 7.40 (ddd, J = 8.5, 4.7, 4.0 Hz, 1H), 7.02 (d, J = 2.9 Hz, 1H), 4.10 (s, 3H), 2.48-2.31 (m, 1H), 1.71 (dd, J = 7.2, 4.8 Hz, 1H), |
| | | | | | | 1.27 (d, J = 6.3 Hz, 3H), |
| | | | | | | 1.18 (dd, J = 8.9, 4.8 Hz, |
| | | | | | | 1H). |
| 511 | 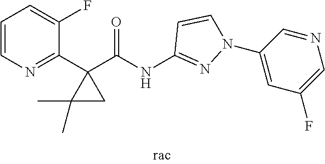 [SMILES: CC1(C)CC1(C(=O)Nc1ccn(-c2cncc(F)c2)n1)c1ncccc1F] | AD | 29 | 0.31 | 370.18 | 1H NMR (400 MHz, Methanol-d4) δ 8.88 (dd, J = 2.1, 0.9 Hz, 1H), 8.60 (dt, J = 4.8, 1.3 Hz, 1H), 8.38 (d, J = 2.5 Hz, 1H), 8.29 (d, J = 2.7 Hz, 1H), 8.09 (dt, J = 10.0, 2.4 Hz, 1H), 7.82 (ddd, J = 10.0, 8.5, 1.4 Hz, 1H), 7.59 (ddd, J = 8.7, 4.9, 4.0 Hz, 1H), 6.90 (d, J = 2.8 Hz, 1H), 1.99 (d, J = 5.8 Hz, |
| | | | | | | 1H), 1.46 (dd, J = 5.9, 2.3 |
| | | | | | | Hz, 1H), 1.33 (s, 3H), 0.94 |
| | | | | | | (s, 3H). |
| 512 | 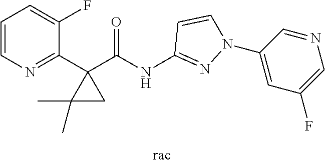 [SMILES: CC1(C)CC1(C(=O)Nc1ccn(-c2cncc(F)c2)n1)c1ncccc1F] | AD | 26 | 1.1 | 386.16 | 1H NMR (400 MHz, Methanol-d4) δ 8.92 (dd, J = 2.3, 0.4 Hz, 1H), 8.59 (dt, J = 4.8, 1.3 Hz, 1H), 8.46- 8.41 (m, 1H), 8.31-8.25 (m, 2H), 7.76 (ddd, J = 10.2, 8.5, 1.4 Hz, 1H), 7.54 (ddd, J = 8.6, 4.8, 4.0 Hz, 1H), 6.89 (d, J = 2.7 Hz, 1H), 1.97 (d, J = 5.7 Hz, 1H), 1.44 (dd, J = 5.8, 2.3 |
| | | | | | | Hz, 1H), 1.32 (s, 3H), 0.92 |
| | | | | | | (s, 3H). |
| 513 | 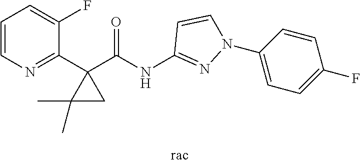 [SMILES: CC1(C)CC1(C(=O)Nc1ccn(-c2ccc(F)cc2)n1)c1ncccc1F] | AD | 21 | 0.98 | 369.19 | 1H NMR (400 MHz, Methanol-d4) δ 8.56 (dt, J = 4.7, 1.4 Hz, 1H), 8.06 (d, J = 2.6 Hz, 1H), 7.73-7.61 (m, 3H), 7.47 (ddd, J = 8.5, 4.7, 3.9 Hz, 1H), 7.24- 7.13 (m, 2H), 6.79 (d, J = 2.6 Hz, 1H), 1.92 (d, J = 5.6 Hz, 1H), 1.40 (dd, J = 5.6, 2.4 Hz, 1H), 1.31 (s, 3H), 0.90 (s, 3H). |
| 514 | 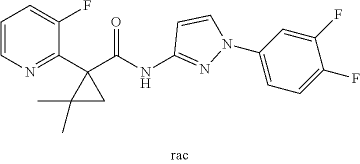 [SMILES: CC1(C)CC1(C(=O)Nc1ccn(-c2ccc(F)c(F)c2)n1)c1ncccc1F] | AD | 25 | 1.8 | 387.15 | 1H NMR (400 MHz, Methanol-d4) δ 8.57 (dt, J = 4.8, 1.4 Hz, 1H), 8.11 (d, J = 2.6 Hz, 1H), 7.75-7.63 (m, 2H), 7.50 (dddd, J = 7.4, 4.7, 3.8, 2.1 Hz, 2H), 7.34 (dt, J = 10.2, 8.7 Hz, 1H), 6.81 (d, J = 2.7 Hz, 1H), 1.93 (d, J = 5.6 Hz, 1H), 1.41 (dd, J = 5.6, 2.4 Hz, 1H), 1.31 (s, 3H), 0.91 |
| | | | | | | (s, 3H). |
| 515 | 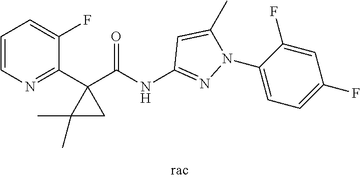 [SMILES: Cc1cc(NC(=O)C2(c3ncccc3F)CC2(C)C)nn1-c1ccc(F)cc1F] | AD | 24 | >5 | 401.14 | 1H NMR (400 MHz, Methanol-d4) δ 8.53 (dt, J = 4.8, 1.3 Hz, 1H), 7.70 (ddd, J = 10.4, 8.4, 1.4 Hz, 1H), 7.55-7.44 (m, 2H), 7.23 (ddd, J = 10.3, 8.7, 2.8 Hz, 1H), 7.18-7.08 (m, 1H), 6.58 (q, J = 0.8 Hz, 1H), 2.15 (dd, J = 1.2, 0.8 Hz, 3H), 1.94 (d, J = 5.6 Hz, 1H), 1.41 (dd, J = 5.6, 2.5 Hz, 1H), 1.30 (s, 3H), 0.89 |
| | | | | | | (s, 3H). |
| 516 | 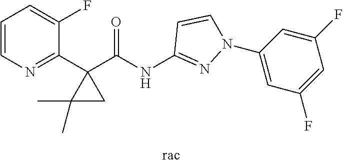 [SMILES: CC1(C)CC1(C(=O)Nc1ccn(-c2cc(F)cc(F)c2)n1)c1ncccc1F] | AD | 16 | 1.3 | 387.15 | 1H NMR (400 MHz, Methanol-d4) δ 8.57 (dt, J = 4.7, 1.4 Hz, 1H), 8.19 (d, J = 2.7 Hz, 1H), 7.69 (ddd, J = 10.4, 8.4, 1.4 Hz, 1H), 7.49 (ddd, J = 8.5, 4.7, 4.0 Hz, 1H), 7.45-7.33 (m, 2H), 6.89-6.78 (m, 2H), 1.93 (d, J = 5.6 Hz, 1H), 1.41 (dd, J = 5.6, 2.4 Hz, 1H), 1.31 (s, 3H), 0.91 (s, |
| | | | | | | 3H). |
| 517 | 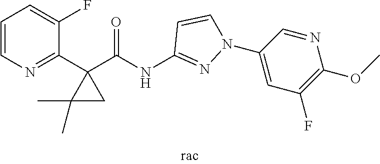 [SMILES: COc1ncc(-n2ccc(NC(=O)C3(c4ncccc4F)CC3(C)C)n2)cc1F] | AD | 16 | 1.1 | 400.20 | 1H NMR (400 MHz, Methanol-d4) δ 8.57 (dt, J = 4.8, 1.3 Hz, 1H), 8.30 (d, J = 2.4 Hz, 1H), 8.08 (d, J = 2.6 Hz, 1H), 7.91 (dd, J = 11.0, 2.3 Hz, 1H), 7.70 (ddd, J = 10.4, 8.4, 1.4 Hz, 1H), 7.50 (ddd, J = 8.5, 4.7, 4.0 Hz, 1H), 6.81 (d, J = 2.6 Hz, 1H), 4.02 (s, 3H), 1.93 (d, J = 5.6 Hz, 1H), 1.41 |
| | | | | | | (dd, J = 5.7, 2.4 Hz, 1H), |
| | | | | | | 1.31 (s, 3H), 0.91 (s, 3H). |
| 518 | 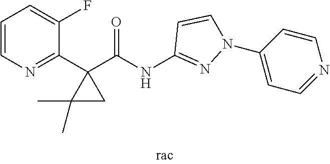 [SMILES: CC1(C)CC1(C(=O)Nc1ccn(-c2ccncc2)n1)c1ncccc1F] | AD | 2 | 1.2 | 352.13 | 1H NMR (400 MHz, Methanol-d4) δ 8.80- 8.72 (m, 1H), 8.62-8.52 (m, 2H), 8.33-8.25 (m, 2H), 7.67 (ddd, J = 10.5, 8.4, 1.4 Hz, 1H), 7.48 (ddd, J = 8.5, 4.7, 3.9 Hz, 1H), 7.14 (d, J = 3.0 Hz, 1H), 1.93 (d, J = 5.6 Hz, 1H), 1.44 (dd, J = 5.7, 2.4 Hz, 1H), 1.30 (s, 3H), 0.91 (s, |
| | | | | | | 3H). |
| 519 | 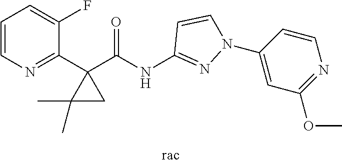 [SMILES: COc1cc(-n2ccc(NC(=O)C3(c4ncccc4F)CC3(C)C)n2)ccn1] | AD | 6 | >2 | 382.20 | 1H NMR (400 MHz, Methanol-d4) δ 8.57 (dt, J = 4.8, 1.3 Hz, 1H), 8.49 (d, J = 2.9 Hz, 1H), 8.24 (d, J = 6.6 Hz, 1H), 7.72 (ddd, J = 10.2, 8.4, 1.4 Hz, 1H), 7.66 (dd, J = 6.6, 2.1 Hz, 1H), 7.56-7.47 (m, 2H), 7.03 (d, J = 2.9 Hz, 1H), 5.49 (s, 1H), 4.17 (s, 3H), 1.95 (d, J = 5.7 Hz, 1H), 1.44 (dd, |
| | | | | | | J = 5.7, 2.3 Hz, 1H), 1.31 (s, |
| | | | | | | 3H), 0.92 (s, 3H). |
| 520 | 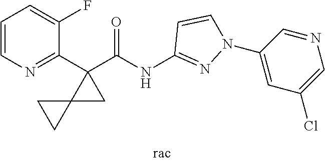 [SMILES: O=C(Nc1ccn(-c2cncc(Cl)c2)n1)C1(c2ncccc2F)CC12CC2] | AD | 45 | 1.6 | 384.08 | 1H NMR (400 MHz, Methanol-d4) δ 8.94 (d, J = 2.3 Hz, 1H), 8.53-8.41 (m, 2H), 8.35-8.25 (m, 2H), 7.82 (ddd, J = 9.7, 8.4, 1.4 Hz, 1H), 7.63-7.54 (m, 1H), 6.87 (d, J = 2.7 Hz, 1H), 2.28 (d, J = 5.0 Hz, 1H), 2.18 (d, J = 5.0 Hz, 1H), 1.40-1.22 (m, 2H), 1.11-0.96 (m, 1H), 0.89 |
| | | | | | | (dt, J = 10.3, 5.4 Hz, 1H). |
| 521 | 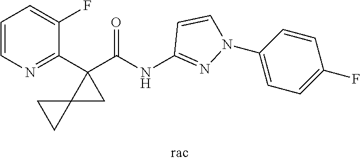 [SMILES: O=C(Nc1ccn(-c2ccc(F)cc2)n1)C1(c2ncccc2F)CC12CC2] | AD | 48 | 2.3 | 367.13 | 1H NMR (400 MHz, Methanol-d4) δ 8.48 (dt, J = 4.9, 1.3 Hz, 1H), 8.07 (d, J = 2.6 Hz, 1H), 7.81 (ddd, J = 9.8, 8.4, 1.3 Hz, 1H), 7.76-7.65 (m, 2H), 7.58 (ddd, J = 8.4, 4.9, 4.3 Hz, 1H), 7.24-7.13 (m, 2H), 6.76 (d, J = 2.6 Hz, 1H), 5.49 (s, 1H), 2.26 (d, J = 5.0 Hz, 1H), 2.19-2.13 (m, |
| | | | | | | 1H), 1.30 (ddd, J = 8.6, 6.3, |
| | | | | | | 1.9 Hz, 2H), 1.09-0.99 (m, |
| | | | | | | 1H), 0.88 (ddd, J = 7.9, 6.1, |
| | | | | | | 4.6 Hz, 1H). |
| 522 | 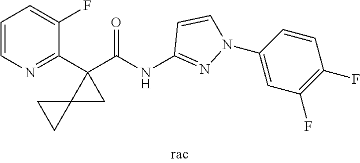 [SMILES: O=C(Nc1ccn(-c2ccc(F)c(F)c2)n1)C1(c2ncccc2F)CC12CC2] | AD | 41 | 0.81 | 385.09 | 1H NMR (400 MHz, Methano1-d4) δ 8.46 (dt, J = 4.9, 1.3 Hz, 1H), 8.12 (d, J = 2.7 Hz, 1H), 7.80-7.65 (m, 2H), 7.58-7.46 (m, 2H), 7.34 (dt, J = 10.2, 8.8 Hz, 1H), 6.79 (d, J = 2.6 Hz, 1H), 2.24 (d, J = 4.9 Hz, 1H), 2.15 (d, J = 4.9 Hz, 1H), 1.35-1.23 (m, 2H), 1.03 (dt, J = 9.4, 5.4 Hz, |
| | | | | | | 1H), 0.92-0.82 (m, 1H). |
| 523 | 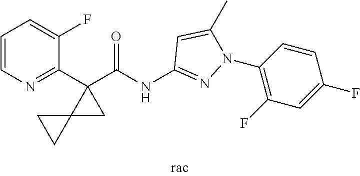 [SMILES: Cc1cc(NC(=O)C2(c3ncccc3F)CC23CC3)nn1-c1ccc(F)cc1F] | AD | 32 | >2 | 399.12 | 1H NMR (400 MHz, Methanol-d4) δ 8.51 (dt, J = 5.0, 1.2 Hz, 1H), 7.92 (ddd, J = 9.7, 8.5, 1.3 Hz, 1H), 7.66 (ddd, J = 8.5, 5.0, 4.3 Hz, 1H), 7.51 (td, J = 8.7, 5.8 Hz, 1H), 7.24 (ddd, J = 10.3, 8.7, 2.7 Hz, 1H), 7.14 (dddd, J = 8.9, 8.0, 2.8, 1.5 Hz, 1H), 6.53 (d, J = 0.9 Hz, 1H), 2.27 (d, J = 5.0 Hz, 1H), 2.21-2.13 (m, |
| | | | | | | 4H), 1.30 (ddd, J = 7.6, 6.2, |
| | | | | | | 1.7 Hz, 2H), 1.06 (dt, J = |
| | | | | | | 8.8, 5.7 Hz, 1H), 0.95- |
| | | | | | | 0.85 (m, 1H). |
| 524 | 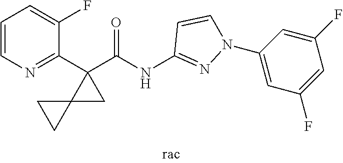 [SMILES: O=C(Nc1ccn(-c2cc(F)cc(F)c2)n1)C1(c2ncccc2F)CC12CC2] | AD | 23 | 1.5 | 385.12 | 1H NMR (400 MHz, Methanol-d4) δ 8.51 (dt, J = 5.0, 1.2 Hz, 1H), 7.92 (ddd, J = 9.7, 8.5, 1.3 Hz, 1H), 7.66 (ddd, J = 8.5, 5.0, 4.3 Hz, 1H), 7.51 (td, J = 8.7, 5.8 Hz, 1H), 7.24 (ddd, J = 10.3, 8.7, 2.7 Hz, 1H), 7.14 (dddd, J = 8.9, 8.0, 2.8, 1.5 Hz, 1H), 6.53 (d, J = 0.9 Hz, 1H), 2.27 (d, J = |
| | | | | | | 5.0 Hz, 1H), 2.21-2.13 (m, |
| | | | | | | 4H), 1.30 (ddd, J = 7.6, 6.2, |
| | | | | | | 1.7 Hz, 2H), 1.06 (dt, J = |
| | | | | | | 8.8, 5.7 Hz, 1H), 0.95- |
| | | | | | | 0.85 (m, 1H). |
| 525 | 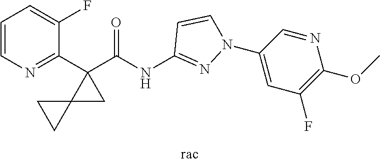 [SMILES: COc1ncc(-n2ccc(NC(=O)C3(c4ncccc4F)CC34CC4)n2)cc1F] | AD | 7 | 0.68 | 398.08 | 1H NMR (400 MHz, Methanol-d4) δ 8.63 (dd, J = 5.3, 1.2 Hz, 1H), 8.34 (d, J = 2.4 Hz, 1H), 8.24-8.14 (m, 1H), 8.12 (d, J = 2.6 Hz, 1H), 7.95 (dd, J = 11.0, 2.4 Hz, 1H), 7.87 (ddd, J = 8.6, 5.3, 4.5 Hz, 1H), 6.77 (d, J = 2.6 Hz, 1H), 4.02 (s, 3H), 2.41 (d, J = 5.4 Hz, 1H), 2.22 (d, J = 5.3 Hz, |
| | | | | | | 1H), 1.43-1.30 (m, 2H), |
| | | | | | | 1.12 (dt, J = 10.1, 5.3 Hz, |
| | | | | | | 1H), 0.97 (dt, J = 10.7, 5.5 |
| | | | | | | Hz, 1H). |
| 526 | 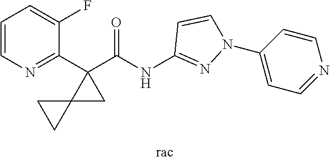 [SMILES: O=C(Nc1ccn(-c2ccncc2)n1)C1(c2ncccc2F)CC12CC2] | AD | 31 | 1.9 | 350.15 | 1H NMR (400 MHz, Methanol-d4) δ 8.80- 8.72 (m, 2H), 8.59 (d, J = 3.0 Hz, 1H), 8.42 (dt, J = 4.8, 1.4 Hz, 1H), 8.33- 8.25 (m, 2H), 7.65 (ddd, J = 9.9, 8.4, 1.4 Hz, 1H), 7.47 (ddd, J = 8.4, 4.8, 4.2 Hz, 1H), 7.12 (d, J = 3.0 Hz, 1H), 2.24 (d, J = 4.9 Hz, 1H), 2.17 (d, J = 4.9 Hz, |
| | | | | | | 1H), 1.33-1.20 (m, 2H), |
| | | | | | | 1.02 (dt, J = 9.9, 5.2 Hz, |
| | | | | | | 1H), 0.92-0.82 (m, 1H). |
| 527 | 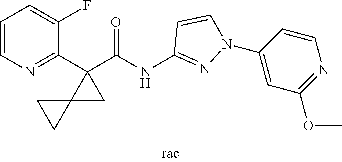 [SMILES: COc1cc(-n2ccc(NC(=O)C3(c4ncccc4F)CC34CC4)n2)ccn1] | AD | 42 | >2 | 380.17 | 1H NMR (400 MHz, Methanol-d4) δ 8.51- 8.40 (m, 2H), 8.22 (dd, J = 6.4, 0.4 Hz, 1H), 7.69 (ddd, J = 9.8, 8.4, 1.4 Hz, 1H), 7.61 (dd, J = 6.5, 2.1 Hz, 1H), 7.57-7.39 (m, 2H), 6.98 (d, J = 2.9 Hz, 1H), 4.12 (s, 3H), 2.24 (d, J = 4.9 Hz, 1H), 2.17 (d, J = 4.9 Hz, 1H), 1.28 (dddd, J = |
| | | | | | | 12.0, 9.0, 6.0, 4.7 Hz, 3H), |
| | | | | | | 1.03 (dt, J = 9.9, 5.3 Hz, |
| | | | | | | 1H), 0.87 (dt, J = 8.7, 5.2 |
| | | | | | | Hz, 1H). |
| 528 | 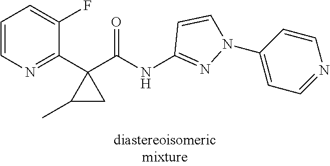 [SMILES: CC1CC1(C(=O)Nc1ccn(-c2ccncc2)n1)c1ncccc1F] | AD | 4 | 0.27 | 338.13 | 1H NMR (400 MHz, Methanol-d4) δ 8.78- 8.70 (m, 2H), 8.59 (d, J = 3.0 Hz, 1H), 8.39 (dt, J = 4.7, 1.5 Hz, 1H), 8.31- 8.22 (m, 2H), 7.57 (ddd, J = 10.5, 8.3, 1.4 Hz, 1H), 7.38 (ddd, J = 8.5, 4.7, 4.0 Hz, 1H), 7.17 (d, J = 3.0 Hz, 1H), 2.49-2.36 (m, 1H), 1.70 (dd, J = 7.2, 4.8 Hz, |
| | | | | | | 1H), 1.27 (d, J = 6.4 Hz, |
| | | | | | | 3H), 1.18 (dd, J = 8.9, 4.8 |
| | | | | | | Hz, 1H). |
| 529 | 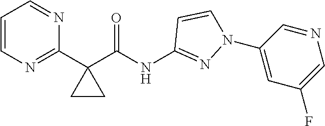 [SMILES: O=C(Nc1ccn(-c2cncc(F)c2)n1)C1(c2ncccn2)CC1] | AD | 40 | 1.3 | 325.12 | 1H NMR (400 MHz, Methanol-d4) δ 8.91 (dd, J = 2.3, 1.0 Hz, 1H), 8.80 (d, J = 5.0 Hz, 2H), 8.38 (d, J = 2.6 Hz, 1H), 8.32 (d, J = 2.7 Hz, 1H), 8.09 (dt, J = 10.0, 2.3 Hz, 1H), 7.34 (t, J = 4.9 Hz, 1H), 6.98 (d, J = 2.7 Hz, 1H), 1.92-1.78 (m, 4H). |
| 530 | 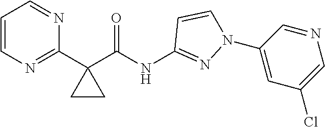 [SMILES: O=C(Nc1ccn(-c2cncc(Cl)c2)n1)C1(c2ncccn2)CC1] | AD | 42 | >5 | 341.05 | 1H NMR (400 MHz, Methanol-d4) δ 8.97 (d, J = 2.3 Hz, 1H), 8.80 (d, J = 4.9 Hz, 2H), 8.44 (d, J = 2.1 Hz, 1H), 8.31 (dd, J = 4.0, 2.4 Hz, 2H), 7.34 (t, J = 4.9 Hz, 1H), 6.97 (d, J = 2.7 Hz, 1H), 1.92-1.78 (m, 4H). |
| 531 | 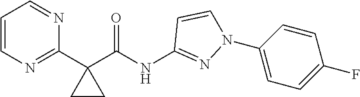 [SMILES: O=C(Nc1ccn(-c2ccc(F)cc2)n1)C1(c2ncccn2)CC1] | AD | 53 | RND | 324.13 | 1H NMR (400 MHz, Methanol-d4) δ 8.79 (d, J = 4.9 Hz, 2H), 8.10 (d, J = 2.6 Hz, 1H), 7.79-7.69 (m, 2H), 7.34 (t, J = 4.9 Hz, 1H), 7.27-7.16 (m, 2H), 6.87 (d, J = 2.6 Hz, 1H), |
| | | | | | | 1.92-1.78 (m, 4H). |
| 532 | 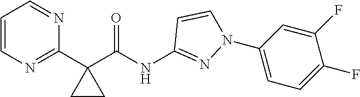 [SMILES: O=C(Nc1ccn(-c2ccc(F)c(F)c2)n1)C1(c2ncccn2)CC1] | AD | 26 | >5 | 342.09 | 1H NMR (400 MHz, Methanol-d4) δ 8.79 (d, J = 4.9 Hz, 2H), 8.15 (d, J = 2.7 Hz, 1H), 7.74 (ddd, J = 11.9, 7.0, 2.7 Hz, 1H), 7.55 (dddd, J = 9.0, 3.7, 2.7, 1.7 Hz, 1H), 7.43-7.30 (m, |
| | | | | | | 2H), 6.89 (d, J = 2.6 Hz, |
| | | | | | | 1H), 1.91-1.78 (m, 4H). |
| 533 | 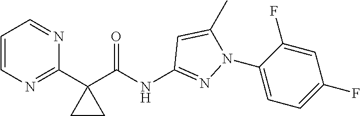 [SMILES: Cc1cc(NC(=O)C2(c3ncccn3)CC2)nn1-c1ccc(F)cc1F] | AD | 13 | >2 | 356.13 | 1H NMR (400 MHz, Methanol-d4) δ 8.75 (d, J = 5.0 Hz, 2H), 7.56 (td, J = 8.7, 5.8 Hz, 1H), 7.35- 7.21 (m, 2H), 7.17 (tdd, J = 8.9, 2.8, 1.5 Hz, 1H), 6.67 (d, J = 0.9 Hz, 1H), 2.19 (t, J = 1.0 Hz, 3H), 1.92-1.77 |
| | | | | | | (m, 4H). |
| 534 | 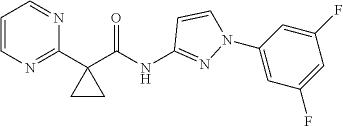 [SMILES: O=C(Nc1ccn(-c2cc(F)cc(F)c2)n1)C1(c2ncccn2)CC1] | AD | 13 | >5 | 342.14 | 1H NMR (400 MHz, DMSO-d6) δ 12.18 (s, 1H), 8.82 (d, J = 5.0 Hz, 2H), 8.55 (d, J = 2.7 Hz, 1H), 7.60-7.53 (m, 2H), 7.49- 7.41 (m, 1H), 7.20-7.11 (m, 1H), 6.90 (d, J = 2.6 Hz, 1H), 1.74-1.57 (m, 5H). |
| 535 | 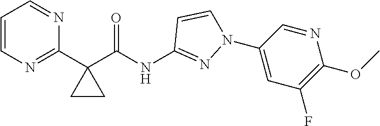 [SMILES: COc1ncc(-n2ccc(NC(=O)C3(c4ncccn4)CC3)n2)cc1F] | AD | 8 | >5 | 355.10 | 1H NMR (400 MHz, Methanol-d4) δ 8.79 (d, J = 5.0 Hz, 2H), 8.36 (d, J = 2.3 Hz, 1H), 8.13 (d, J = 2.6 Hz, 1H), 7.97 (dd, J = 11.0, 2.4 Hz, 1H), 7.34 (t, J = 4.9 Hz, 1H), 6.90 (d, J = 2.6 Hz, 1H), 4.04 (s, 3H), 1.92- 1.80 (m, 4H). |
| 536 | 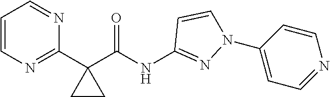 [SMILES: O=C(Nc1ccn(-c2ccncc2)n1)C1(c2ncccn2)CC1] | AD | 45 | >5 | 307.16 | ¹H NMR (400 MHz, DMSO- d₆) δ 12.22 (s, 1H), 8.85- 8.68 (m, 4H), 8.02 (s, 2H), 7.63 (ddd, J = 33.1, 5.7, 3.4 Hz, 1H), 7.42 (td, J = 4.9, 1.6 Hz, 1H), 7.04 (d, J = 2.7 Hz, 1H), 1.71-1.62 |
| | | | | | | (m, 3H), 1.62-1.48 (m, 2H). |

*The Average HEK293 IC50 reflects the average of one or more replicates.
RND = not determined yet

### Example 1.4. Compounds Prepared Using Copper-Mediated Aryl Coupling as Final Step

Described below are Scheme Aryl-1 and Scheme Aryl-2 (includes methods A-D).

### Scheme Aryl-1 (Synthesis of Common Intermediate 1-(2-fluorophenyl)-N-(1H-pyrazol-3-yl)cyclopropane-1-carboxamide for Copper Coupling Procedures)

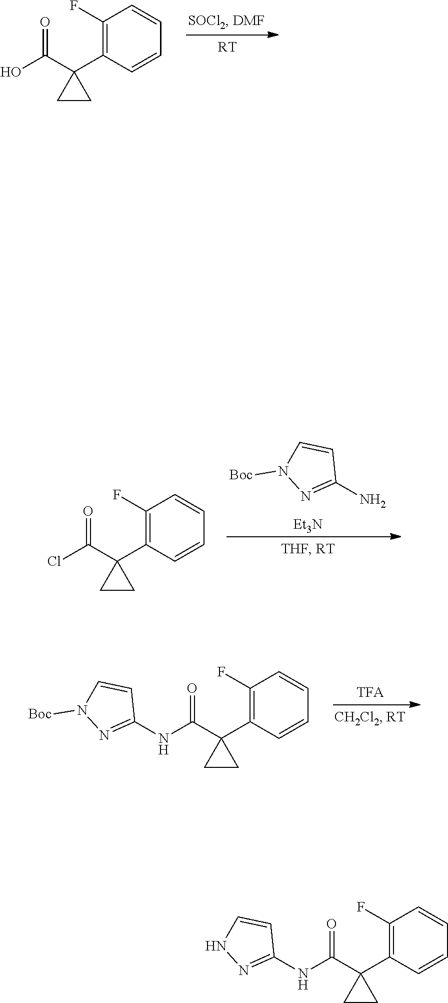

### Step 1: 1-(2-fluorophenyl)cyclopropane-1-carbonyl chloride

1-(2-fluorophenyl)cyclopropanecarboxylic acid (5.0 g, 27.47 mmol) and thionyl chloride (6.0 mL, 82.26 mmol) were combined at room temperature under nitrogen atmosphere. To the resultant brown suspension was added N,N-dimethylformamide (approximately 2 μL, 0.02 mmol), and the reaction mixture was stirred at room temperature for 16 h. Excess thionyl chloride and HCl were removed via rotary evaporation. The crude residue was azeotrope-dried with toluene, and the sample was used in the next step without further purification.

### Step 2: tert-butyl 3-(1-(2-fluorophenyl)cyclopropane-1-carboxamido)-1H-pyrazole-1-carboxylate

The crude residue prepared in Step 1 was dissolved in THF (34.0 mL). Triethylamine (7.76 mL, 55.68 mmol) was added, followed by tert-butyl 3-aminopyrazole-1-carboxylate (4.25 g, 23.20 mmol). The resultant reaction mixture was stirred for 16 h at room temperature. The reaction mixture was partitioned between ethyl acetate (100 mL) and saturated aqueous NaHCO₃. The layers were separated, and the aqueous phase was further extracted with ethyl acetate (2×125 mL). The combined organics were washed with water (200 mL) and brine (200 mL), dried (Na₂SO₄), filtered, and concentrated. The crude residue was purified by silica gel chromatography (330 g column; linear gradient of 0-15% ethyl acetate/heptane) to provide tert-butyl 3-(1-(2-fluorophenyl)cyclopropane-1-carboxamido)-1H-pyrazole-1-carboxylate, the entirety of which was carried forward to Step 3. 1H NMR (400 MHz, DMSO-d₆) δ 9.87 (s, 1H), 8.14 (d, J=2.9 Hz, 1H), 7.49-7.32 (m, 2H), 7.26-7.11 (m, 2H), 6.78 (d, J=2.9 Hz, 1H), 1.59 (q, J=4.4 Hz, 2H), 1.54 (s, 9H), 1.15 (q, J=4.4 Hz, 2H) ppm. ESI-MS m/z calc. 345.15, found 346.12 (M+1).

### Step 3: 1-(2-fluorophenyl)-N-(1H-pyrazol-3-yl)cyclopropane-1-carboxamide

tert-Butyl 3-(1-(2-fluorophenyl)cyclopropane-1-carboxamido)-1H-pyrazole-1-carboxylate obtained in Step 2 was dissolved in dichloromethane (50.0 mL). To the resultant solution was added TFA (5.0 mL, 64.90 mmol), and the reaction mixture was stirred for 16 h at room temperature. The solvent was removed in vacuo, and the crude residue was dissolved in dichloromethane and washed with saturated aqueous NaHCO₃ solution. The layers were separated using a phase separation cartridge. The organic phase was concentrated, then lyophilized to provide 1-(2-fluorophenyl)-N-(1H-pyrazol-3-yl)cyclopropane-1-carboxamide (5.21 g, 92% yield). 1H NMR (300 MHz, CDCl₃) δ 9.76 (s, 3H), 8.88 (s, 1H), 7.56 (m, 1H), 7.48-7.34 (m, 2H), 7.24-7.06 (m, 2H), 6.69 (m, 1H), 1.93-1.73 (m, 2H), 1.26 (m, 2H) ppm. ESI-MS m/z calc. 352.13, found 353.17 (M+1).

### Scheme Aryl-2. General Coupling Procedure for Preparation of Compounds in Table B

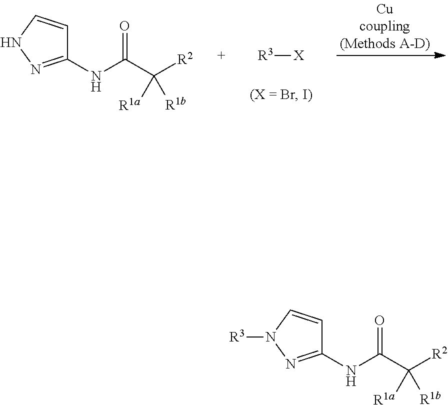

Scheme Aryl-2 provides a general synthetic route for the preparation of compounds listed in Table B. Using 1-(2-fluorophenyl)-N-(1H-pyrrol-3-yl)cyclopropane-1-carboxamide and the appropriate selection of aryl bromide or aryl iodide, compounds within Table B were synthesized according to one of several copper coupling procedures (Copper Coupling Methods A through D). A representative procedure is provided for each method. The coupling method used, as well as the reaction yield and characterization information for each compound is listed within Table B.

### Copper Coupling Method A

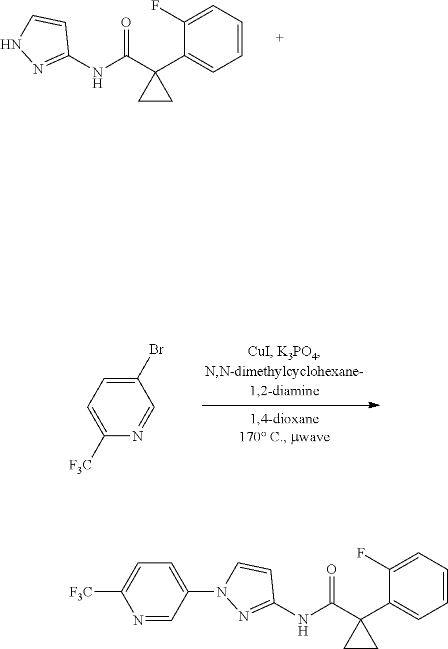 [SMILES: O=C(Nc1cc[nH]n1)C1(c2ccccc2F)CC1 ; FC(F)(F)c1ccc(Br)cn1 ; "CuI, K3PO4,N,N-dimethylcyclohexane-1,2-diamine" ; O=C(Nc1ccn(-c2ccc(C(F)(F)F)nc2)n1)C1(c2ccccc2F)CC1]

### 1-(2-fluorophenyl)-N-(1-(6-(trifluoromethyl)pyridin-3-yl)-1H-pyrazol-3-yl)cyclopropane-1-carboxamide (Compound 315)

1-(2-fluorophenyl)-N-(1H-pyrazol-3-yl)cyclopropanecarboxamide (30 mg, 0.12 mmol, 1.0 eq), 5-bromo-2-(trifluoromethyl)pyridine (100 mg, 0.44 mmol, 3.7 eq), CuI (15 mg, 0.08 mmol, 0.66 eq), N,N-dimethylcyclohexane-1,2-diamine (6 mg. 0.04 mmol, 0.33 eq), tripotassium phosphate (100 mg 3.9 eq) and 1,4-dioxane (1.5 mL) were combined under nitrogen and heated to 170° C. in a microwave for 15 minutes. To the reaction mixture was added 1:1 water/concentrated ammonium hydroxide (2 mL) and ethyl acetate (5 mL). The layers were separated, and the aqueous phase was further extracted with ethyl acetate. The combined organic extracts were dried (Na₂SO₄), filtered, and concentrated. The crude residue was purified by silica gel chromatography (ethyl acetate/heptane) to provide 1-(2-fluorophenyl)-N-(1-(6-(trifluoromethyl)pyridin-3-yl)-1H-pyrazol-3-yl)cyclopropane-1-carboxamide (7.2 mg, 15% yield).

### Copper Coupling Method B

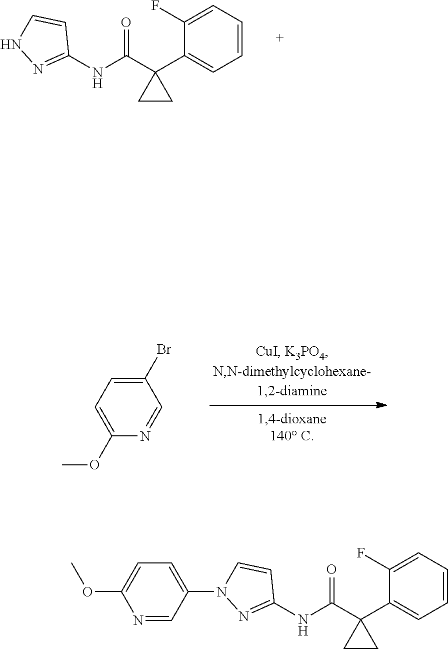

### 1-(2-fluorophenyl)-N-(1-(6-methoxypyridin-3-yl)-1H-pyrazol-3-yl)cyclopropane-1-carboxamide (Compound 319)

1-(2-fluorophenyl)-N-(1H-pyrazol-3-yl)cyclopropanecarboxamide (40.0 mg, 0.16 mmol, 1.0 eq), 5-bromo-2-methoxypyridine (30 μL, 0.23 mmol, 1.4 eq), copper (I) iodide (15.5 mg, 0.08 mmol, 0.5 eq), tripotassium phosphate (69 mg, 0.33 mmol, 2.0 eq), N,N-dimethylcyclohexane-1,2-diamine (13 μL, 0.08 mmol, 0.5 eq), and 1,4-dioxane (2.0 mL) were combined. The reaction vessel was sealed and heated thermally to 140° C. for 16 h. The reaction mixture was cooled to room temperature and partitioned between dichloromethane and saturated aqueous NH₄Cl. The layers were separated on a phase separation cartridge. The organics were concentrated, and the crude residue purified by C18 preparatory HPLC (acetonitrile/water with TFA modifier). The material thus obtained was dissolved in dichloromethane and washed with saturated aqueous NaHCO₃. The organics were separated and concentrated to provide 1-(2-fluorophenyl)-N-(1-(6-methoxypyridin-3-yl)-1H-pyrazol-3-yl)cyclopropane-1-carboxamide (30.1 mg, 52% yield).

### Copper Coupling Method C

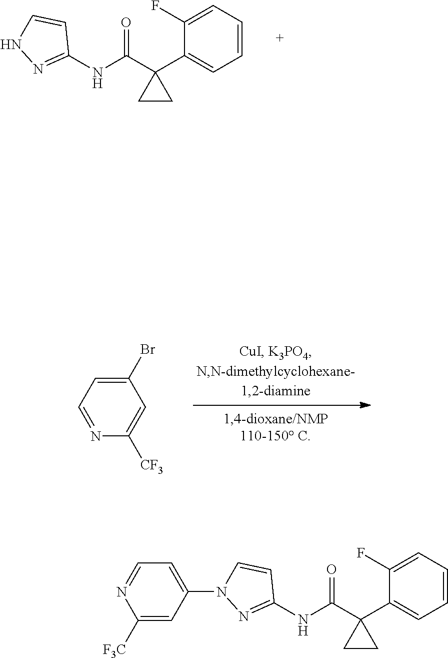 [SMILES: O=C(Nc1cc[nH]n1)C1(c2ccccc2F)CC1 ; FC(F)(F)c1cc(Br)ccn1 ; "CuI, K3PO4,N,N-dimethylcyclohexane-1,2-diamine" ; O=C(Nc1ccn(-c2ccnc(C(F)(F)F)c2)n1)C1(c2ccccc2F)CC1]

### 1-(2-fluorophenyl)-N-(1-(2-(trifluoromethyl)pyridin-4-yl)-1H-pyrazol-3-yl)cyclopropane-1-carboxamide (Compound 324)

1-(2-fluorophenyl)-N-(1H-pyrazol-3-yl)cyclopropanecarboxamide (50.0 mg, 0.20 mmol, 1.0 eq), 4-bromo-2-(trifluoromethyl)pyridine (76 mg, 0.28 mmol, 1.4 eq), copper (I) iodide (20 mg, 0.10 mmol, 0.5 eq), tripotassium phosphate (110 mg, 0.52 mmol, 2.6 eq), N,N-dimethylcyclohexane-1,2-diamine (15 μL, 0.10 mmol, 0.5 eq), and 1,4-dioxane (2.0 mL) were combined. The reaction vessel was sealed and heated thermally to 110° C. for approximately 16 h. NMP (1.0 mL) was added, and heating was continued at 150° C. for approximately 60 h. The reaction mixture was cooled to room temperature and partitioned between dichloromethane and saturated aqueous NH₄Cl. The mixture was filtered through Celite, and the filter pad was rinsed with dichloromethane. The filtrate layers were separated on a phase separation cartridge. The dichloromethane fraction was concentrated, and the crude residue was purified by C18 preparatory HPLC (acetonitrile/water with TFA modifier). The material thus obtained was dissolved in dichloromethane and washed with saturated aqueous NaHCO₃. The organics were separated and concentrated to provide 1-(2-fluorophenyl)-N-(1-(2-(trifluoromethyl)pyridin-4-yl)-1H-pyrazol-3-yl)cyclopropane-1-carboxamide (7.5 mg, 9% yield).

### Copper Coupling Method D

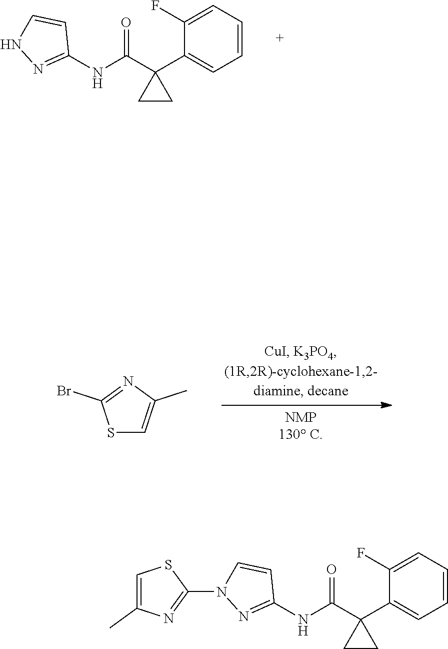

### 1-(2-fluorophenyl)-N-(1-(4-methylthiazol-2-yl)-1H-pyrazol-3-yl)cyclopropane-1-carboxamide (Compound 374)

1-(2-fluorophenyl)-N-(1H-pyrazol-3-yl)cyclopropanecarboxamide (40 mg, 0.16 mmol, 1.0 eq), 2-bromo-4-methylthiazole (29 mg, 0.16 mmol, 1.0 eq), CuI (6.2 mg, 0.03 mmol, 0.2 eq), potassium carbonate (5.6 mg, 0.25 eq), (1R,2R)-cyclohexane-1,2-diamine (3.7 mg, 0.03 mmol, 0.2 eq), decane (13 μL, 0.07 mmol, 0.4 eq) and 1-methyl-pyrrolidin-2-one (3 mL) were combined in a sealed vial and heated to 130° C. for 16 h. The reaction mixture was cooled to room temperature and partitioned between dichloromethane and saturated aqueous NH₄Cl. The organic layer was collected and evaporated to dryness. The crude residue was purified by C18 preparatory HPLC (acetonitrile/water with TFA modifier). The material thus obtained was dissolved in dichloromethane and washed with saturated aqueous NaHCO₃. The organics were separated and concentrated to provide 1-(2-fluorophenyl)-N-(1-(4-methylthiazol-2-yl)-1H-pyrazol-3-yl)cyclopropane-1-carboxamide (4.5 mg, 8% yield).

### Copper Coupling Method E

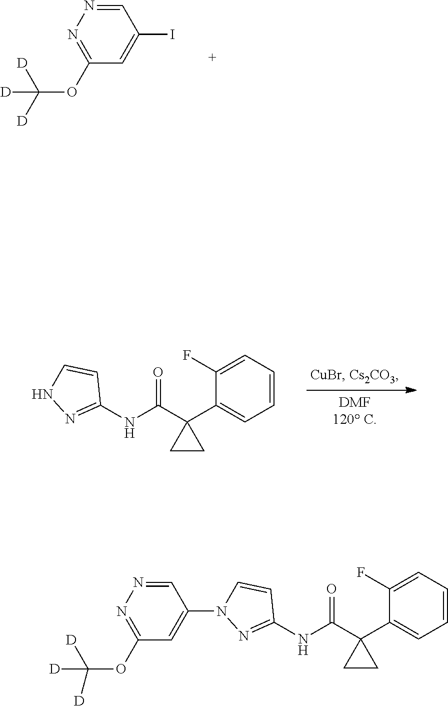 [SMILES: [2H]C([2H])([2H])Oc1cc(I)cnn1 ; O=C(Nc1cc[nH]n1)C1(c2ccccc2F)CC1 ; "CuBr, Cs2CO3," ; [2H]C([2H])([2H])Oc1cc(-n2ccc(NC(=O)C3(c4ccccc4F)CC3)n2)cnn1]

### 1-(2-fluorophenyl)-N-[1-[6-(trideuteriomethoxy)pyridazin-4-yl]pyrazol-3-yl]cyclopropanecarboxamide (Compound 432)

1-(2-fluorophenyl)-N-(1H-pyrazol-3-yl)cyclopropanecarboxamide (50 mg, 0.187 mmol, 1.5 eq), 5-iodo-3-(trideuteriomethoxy)pyridazine (30 mg, 0.126 mmol, 1.0 eq), copper(I) bromide (0 mg, 0.070 mmol, 0.56 eq), cesium carbonate (250 mg, 0.767 mmol, 6.1 eq) and DMF (2.0 mL) were combined. The resultant mixture was heated at 120° C. for 16 hours. The reaction mixture was cooled to room temperature, filtered, and the filtrate directly purified by C18 preparatory HPLC (acetonitrile/water with TFA modifier). The material thus obtained was dissolved in dichloromethane, and the solution was washed with saturated aqueous sodium bicarbonate. The organics were collected and evaporated to provide 1-(2-fluorophenyl)-N-[1-[6-(trideuteriomethoxy)pyridazin-4-yl]pyrazol-3-yl]cyclopropanecarboxamide (2.1 mg, 3% yield).

TABLE B
Compounds Prepared Using Copper-Mediated Aryl Coupling as Final Step

| | | | | Average | | |
|---|---|---|---|---|---|---|
| | | | | HEK293 | | |
| Cpd | | | Yield | IC50 | | NMR |
| No. | Structure | Method | (%) | (uM)* | M + 1 | (shifts in PPM) |
| 315 | 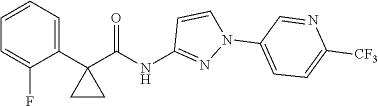 [SMILES: O=C(Nc1ccn(-c2ccc(C(F)(F)F)nc2)n1)C1(c2ccccc2F)CC1] | A | 15 | >5 | 391.14 | 1H NMR (400 MHz, DMSO- d₆) δ 9.67 (s, 1H), 9.19 (d, J = 2.5 Hz, 1H), 8.63 (d, J = 2.7 Hz, 1H), 8.37 (dd, J = 8.5, 2.6 Hz, 1H), 8.01 (d, J = 8.7 Hz, 1H), 7.47 (td, J = 7.9, 1.9 Hz, 1H), 7.39 (tdd, J = |
| | | | | | | 7.4, 5.3, 1.8 Hz, 1H), 7.24- |
| | | | | | | 7.18 (m, 2H), 6.87 (d, J = 2.7 |
| | | | | | | Hz, 1H), 1.61 (q, J = 4.3 Hz, |
| | | | | | | 2H), 1.17 (q, J = 4.4 Hz, 2H). |
| 316 | 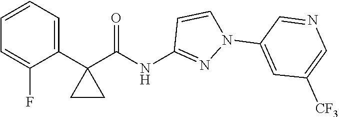 [SMILES: O=C(Nc1ccn(-c2cncc(C(F)(F)F)c2)n1)C1(c2ccccc2F)CC1] | A | 9 | >5 | 391.05 | 1H NMR (400 MHz, DMSO- d₆) δ 9.64 (s, 1H), 9.33 (d, J = 2.4 Hz, 1H), 8.84 (d, J = 1.9 Hz, 1H), 8.66 (d, J = 2.7 Hz, 1H), 8.48 (d, J = 2.3 Hz, 1H), 7.47 (td, J = 7.8, 1.9 Hz, 1H), 7.40 (tdd, J = 7.6, 5.3, 1.8 Hz, 1H), 7.24-7.15 (m, |
| | | | | | | 2H), 6.86 (d, J = 2.7 Hz, 1H), |
| | | | | | | 1.62 (q, J = 4.3 Hz, 2H), 1.17 |
| | | | | | | (q, J = 4.3 Hz, 2H). |
| 317 | 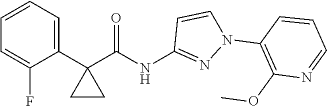 [SMILES: COc1ncccc1-n1ccc(NC(=O)C2(c3ccccc3F)CC2)n1] | B | 30 | >5 | 353.17 | 1H NMR (400 MHz, DMSO- d₆) δ 9.36 (s, 1H), 8.21 (d J = 2.6 Hz, 1H), 8.12 (m, 1H), 7.98 (m, 1H), 7.50-7.35 (m, 2H), 7.24-7.09 (m, 3H), 6.73 (d, J = 2.6 Hz, 1H), 3.97 (s, 3H), 1.60 (m, 2H), |
| | | | | | | 1.15 (m, 2H). |
| 318 | 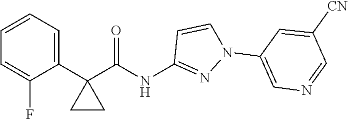 [SMILES: N#Cc1cncc(-n2ccc(NC(=O)C3(c4ccccc4F)CC3)n2)c1] | B | 69 | >5 | 348.16 | 1H NMR (400 MHz, DMSO- d₆) δ 9.55 (s, 1H), 9.27 (d, J = 2.6 Hz, 1H), 8.88 (d, J = 1.7 Hz, 1H), 8.70-8.61 (m, 1H), 8.56 (d, J = 2.7 Hz, 1H), 7.51-7.35 (m, 2H), 7.30- 7.16 (m, 2H), 6.86 (d, J = 2.7 Mz, 1H), 1.61 (m, 2H), 1.17 |
| | | | | | | (m, 2H). |
| 319 | 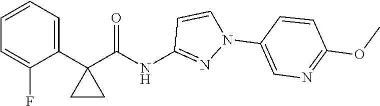 [SMILES: COc1ccc(-n2ccc(NC(=O)C3(c4ccccc4F)CC3)n2)cn1] | B | 52 | 0.058 | 353.17 | 1H NMR (400 MHz, DMSO- d₆) δ 9.40 (s, 1H), 8.53 (d, J = 2.8 Hz, 1H), 8.31 (d, J = 2.6 Hz, 1H), 8.05 (m, 1H), 7.52-7.33 (m, 2H), 7.21 (m, 2H), 6.93 (d, J = 9.0 Hz, 1H), 6.73 (d, J = 2.6 Hz, 1H), 3.87 |
| | | | | | | (s, 3H), 1.60 (m, 2H), 1.15 |
| | | | | | | (m, 2H). |
| 320 | 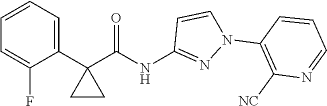 [SMILES: N#Cc1ncccc1-n1ccc(NC(=O)C2(c3ccccc3F)CC2)n1] | B | 18 | >5 | 348.16 | 1H NMR (400 MHz, DMSO- d₆) δ 9.53 (s, 1H), 8.70 (m, 1H), 8.39 (d, J = 2.7 Hz, 1H), 8.23 (m, 1H), 7.87 (m, 1H), 7.51-7.34 (m, 2H), 7.24- 7.16 (m, 2H), 6.88 (d, J = 2.7 Hz, 1H), 1.61 (m, 2H), 1.17 |
| | | | | | | (m, 2H). |
| 321 | 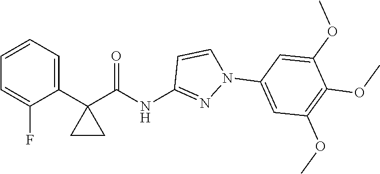 [SMILES: COc1cc(-n2ccc(NC(=O)C3(c4ccccc4F)CC3)n2)cc(OC)c1OC] | B | 3 | >5 | 412.19 | 1H NMR (400 MHz, DMSO- d₆) δ 9.43 (s, 1H), 8.40 (d, J = 2.6 Hz, 1H), 7.48 (t, J = 7.4 Hz, 1H), 7.40 (d, J = 7.1 Hz, 1H), 7.21 (dd, J = 12.2, 6.5 Hz, 2H), 7.02 (s, 2H), 6.73 (d, J = 2.6 Hz, 1H), 3.81 (s, 6H), 3.65 (s, 3H), 1.60 (q, J = 4.4 Hz, 2H), 1.14 (q, J = 4.1 Hz, 2H). |
| 322 | 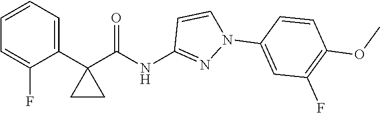 [SMILES: COc1ccc(-n2ccc(NC(=O)C3(c4ccccc4F)CC3)n2)cc1F] | B | 5 | 4.0 | 370.12 | 1H NMR (400 MHz, DMSO- d₆) δ 9.32 (s, 1H), 8.31 (d, J = 2.6 Hz, 1H), 7.63 (dd, J = 12.7, 2.7 Hz, 1H), 7.56-7.45 (m, 2H), 7.44-7.36 (m, 1H), 7.28-7.16 (m, 3H), 6.70 (d, J = 2.5 Hz, 1H), 3.85 (s, 3H), |
| | | | | | | 1.59 (q, J = 4.3 Hz, 2H), 1.15 |
| | | | | | | (q, J = 4.4 Hz, 2H). |
| 323 | 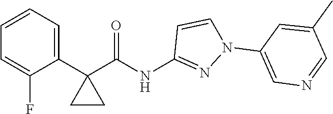 [SMILES: Cc1cncc(-n2ccc(NC(=O)C3(c4ccccc4F)CC3)n2)c1] | B | 65 | 0.35 | 337.15 | 1H NMR (400 MHz, DMSO- d₆) δ 9.42 (s, 1H), 8.81 (d, J = 2.5 Hz, 1H), 8.45 (d, J = 2.7 Hz, 1H), 8.30 (s, 1H), 7.97 (s, 1H), 7.53-7.36 (m, 2H), 7.22 (s, 3H), 6.78 (d, J = 2.6 Hz, 1H), 2.35 (s, 3H), 1.60 (m, 2H), 1.16 (m, 2H). |
| 324 | 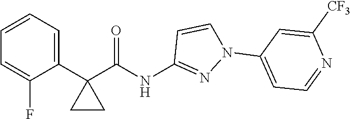 [SMILES: O=C(Nc1ccn(-c2ccnc(C(F)(F)F)c2)n1)C1(c2ccccc2F)CC1] | C | 9 | >5 | 391.09 | 1H NMR (400 MHz, DMSOd₆) δ 9.74 (s, 1H), 8.75 (dd, J = 8.1, 4.2 Hz, 2H), 8.20 (d, J = 2.1 Hz, 1H), 8.03 (dd, J = 5.8, 2.1 Hz, 1H), 7.54-7.36 (m, 2H), 7.28-7.15 (m, 2H), 6.90 (d, J = 2.8 Hz, 1H), 1.62 (q, J = |
| | | | | | | 4.4 Hz, 2H), 1.18 (q, J = 4.4 |
| | | | | | | Hz, 2H). |
| 325 | 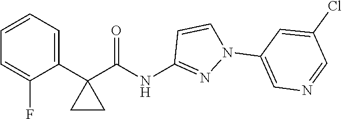 [SMILES: O=C(Nc1ccn(-c2cncc(Cl)c2)n1)C1(c2ccccc2F)CC1] | B | 35 | 0.073 | 357.15 | 1H NMR (400 MHz, DMSO- d₆) δ 9.55 (s, 1H), 8.99 (d, J = 2.3 Hz, 1H), 8.53 (m, 2H), 8.31 (m, 1H), 7.55-7.33 (m, 2H), 7.26-7.16 (m, 2H), 6.83 (d, J = 2.7 Hz, 1H), 1.61 (m, 2H), 1.17 (m, 2H). |
| 326 | 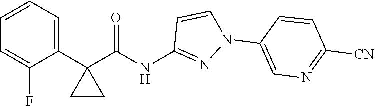 [SMILES: N#Cc1ccc(-n2ccc(NC(=O)C3(c4ccccc4F)CC3)n2)cn1] | B | 14 | >5 | 348.16 | 1H NMR (400 MHz, DMSO- d₆) δ 9.69 (s, 1H), 9.18 (d, J = 2.6 Hz, 1H), 8.64 (d, J = 2.8 Hz, 1H), 8.33 (m, 1H), 8.15 (d, J = 8.6 Hz, 1H), 7.51- 7.35 (m, 2H), 7.27-7.15 (m, 2H), 6.89 (d, J = 2.7 Hz, |
| | | | | | | 1H), 1.62 (m, 2H), 1.17 (m, |
| | | | | | | 2H). |
| 327 | 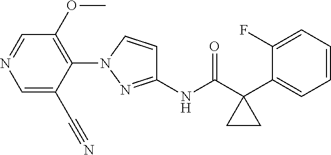 [SMILES: COc1cncc(C#N)c1-n1ccc(NC(=O)C2(c3ccccc3F)CC2)n1] | B | 6 | >5 | 378.18 | 1H NMR (400 MHz, DMSO- d₆) δ 9.52 (s, 1H), 8.11 (d, J = 2.7 Hz, 1H), 7.53-7.34 (m, 2H), 7.20 (m, 2H), 6.84 (d, J = 2.7 Hz, 1H), 3.99 (s, 3H), 1.61 (m, 2H), 1.17 (m, 2H). |
| 328 | 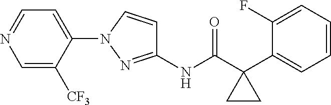 [SMILES: O=C(Nc1ccn(-c2ccncc2C(F)(F)F)n1)C1(c2ccccc2F)CC1] | B | 3 | >5 | 391.15 | 1H NMR (400 MHz, DMSO- d₆) δ 9.46 (s, 1H), 8.07 (d, J = 2.6 Hz, 1H), 7.75 (s, 1H), 7.54-7.32 (m, 2H), 7.21 (m, 2H), 6.84 (d, J = 2.6 Hz, 1H), 1.61 (m, 2H), 1.17 (m, 2H). |
| 329 | 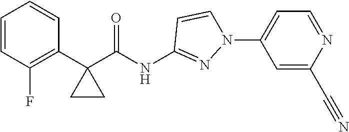 [SMILES: N#Cc1cc(-n2ccc(NC(=O)C3(c4ccccc4F)CC3)n2)ccn1] | B | 56 | 3.6 | 348.16 | 1H NMR (400 MHz, DMSO- d₆) δ 9.66 (s, 1H), 8.70 (m, 2H), 8.39 (d, J = 2.2 Hz, 1H), 8.01 (m, 1H), 7.55-7.36 (m, 2H), 7.29-7.15 (m, 2H), 6.91 (d, J = 2.8 Hz, 1H), 1.62 (m, 2H), 1.18 (m, 2H). |
| 330 | 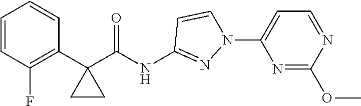 [SMILES: COc1nccc(-n2ccc(NC(=O)C3(c4ccccc4F)CC3)n2)n1] | B | 30 | 0.26 | 354.26 | 1H NMR (300 MHz, CDCl₃) δ 8.51-8.38 (m, 2H), 7.54- 7.39 (m, 2H), 7.26-7.13 (m, 3H), 7.05 (d, J = 2.8 Hz, 1H), 4.06 (s, 3H), 1.82 (m, 2H), 1.28-1.19 (m, 2H). |
| 331 | 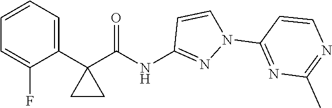 [SMILES: Cc1nccc(-n2ccc(NC(=O)C3(c4ccccc4F)CC3)n2)n1] | B | 24 | >5 | 338.14 | 1H NMR (300 MHz, CDCl₃) δ 8.58 (d, J = 5.7 Hz, 1H), 8.48 (m, 1H), 7.73 (s, 1H), 7.56- 7.35 (m, 3H), 7.24-7.13 (m, 1H), 7.06 (m, 1H), 2.71 (s, 3H), 1.93-1.72 (m, 2H), 1.32-1.13 (m, 2H). |
| 332 | 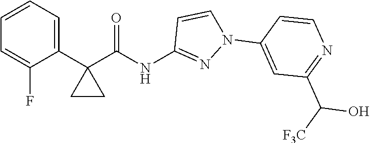 [SMILES: O=C(Nc1ccn(-c2ccnc(C(O)C(F)(F)F)c2)n1)C1(c2ccccc2F)CC1] | B | 15 | RND | 421.17 | 1H NMR (400 MHz, DMSO- d₆) δ 9.71 (s, 1H), 8.60 (m, 2H), 8.00 (d, J = 2.1 Hz, 1H), 7.80 (m, 1H), 7.51-7.36 (m, 2H), 7.28-7.11 (m, 3H), 6.85 (d, J = 2.7 Hz, 1H), 5.14 (m, 1H), 1.62 (m, 2H), 1.17 (m, 2H). |
| 333 | 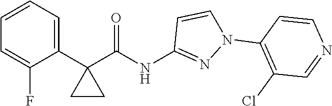 [SMILES: O=C(Nc1ccn(-c2ccncc2Cl)n1)C1(c2ccccc2F)CC1] | B | 10 | 0.95 | 357.15 | 1H NMR (400 MHz, DMSO- d₆) δ 9.61 (s, 1H), 8.79 (s, 1H), 8.58 (d, J = 5.4 Hz, 1H), 8.36 (d, J = 2.7 Hz, 1H), 7.67 (d, J = 5.4 Hz, 1H), 7.52- 7.33 (m, 2H), 7.21 (m, 2H), 6.85 (d, J = 2.7 Hz, 1H), 1.61 |
| | | | | | | (m, 2H), 1.17 (m, 2H). |
| 334 | 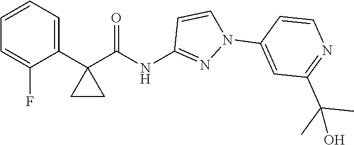 [SMILES: CC(C)(O)c1cc(-n2ccc(NC(=O)C3(c4ccccc4F)CC3)n2)ccn1] | B | 37 | >5 | 381.21 | 1H NMR (400 MHz, DMSO- d₆) δ 9.61 (s, 1H), 8.61- 8.44 (m, 2H), 8.02 (d, J = 2.2 Hz, 1H), 7.59 (m, 1H), 7.52- 7.36 (m, 3H), 7.26-7.16 (m, 2H), 6.82 (d, J = 2.7 Hz, 1H), 5.30 (s, 1H), 1.62 (m, 2H), 1.44 (s, 6H), 1.19- 1.12 (m, 2H). |
| 335 | 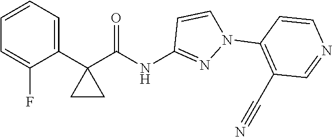 [SMILES: N#Cc1cnccc1-n1ccc(NC(=O)C2(c3ccccc3F)CC2)n1] | B | 17 | >5 | 348.16 | 1H NMR (400 MHz, DMSO- d₆) δ 9.60 (s, 1H), 9.04 (s, 1H), 8.83 (d, J = 5.8 Hz, 1H), 8.57 (d, J = 2.8 Hz, 1H), 7.87 (d, J = 5.8 Hz, 1H), 7.53- 7.34 (m, 2H), 7.30-7.13 (m, 2H), 6.94 (d, J = 2.8 Hz, 1H), 1.62 (m, 2H), 1.19 (m, 2H). |
| 336 | 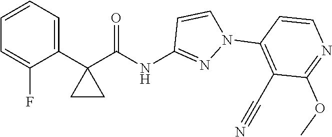 [SMILES: COc1nccc(-n2ccc(NC(=O)C3(c4ccccc4F)CC3)n2)c1C#N] | B | 22 | >5 | 378.18 | 1H NMR (400 MHz, DMSO- d₆) δ 9.64 (s, 1H), 8.53 (d, J = 2.8 Hz, 1H), 8.43 (d, J = 5.9 Hz, 1H), 7.56-7.34 (m, 3H), 7.27-7.16 (m, 2H), 6.93 (d, J = 2.8 Hz, 1H), 4.02 (s, 3H), 1.62 (m, 2H), 1.18 (m, 2H). |
| 337 | 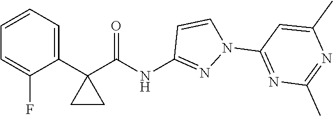 [SMILES: Cc1cc(-n2ccc(NC(=O)C3(c4ccccc4F)CC3)n2)nc(C)n1] | B | 68 | >5 | 352.23 | 1H NMR (400 MHz, DMSO- d₆) δ 9.52 (s, 1H), 8.53 (d, J = 2.8 Hz, 1H), 7.51-7.35 (m, 3H), 7.25-7.19 (m, 2H), 6.84 (d, J = 2.8 Hz, 1H), 2.56 (s, 3H), 2.44 (s, 3H), 1.62-1.57 (m, 2H), 1.18 |
| | | | | | | (m, 2H). |
| 338 | 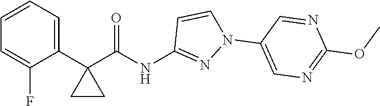 [SMILES: COc1ncc(-n2ccc(NC(=O)C3(c4ccccc4F)CC3)n2)cn1] | B | 3 | >5 | 354.25 | 1H NMR (400 MHz, DMSO- d₆) δ 9.50 (s, 1H), 8.96 (s, 2H), 8.37 (d, J = 2.6 Hz, 1H), 7.47 (td, J = 7.8, 1.8 Hz, 1H), 7.39 (tdd, J = 7.4, 5.2, 1.7 Hz, 1H), 7.25-7.17 (m, 2H), 6.78 (d, J = 2.6 Hz, 1H), 3.94 |
| | | | | | | (s, 3H), 1.59 (q, J = 4.3 Hz, |
| | | | | | | 2H), 1.15 (q, J = 4.3 Hz, 2H). |
| 339 | 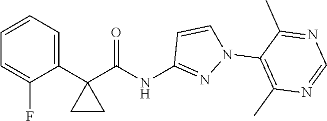 [SMILES: Cc1ncnc(C)c1-n1ccc(NC(=O)C2(c3ccccc3F)CC2)n1] | B | 14 | >5 | 352.22 | 1H NMR (400 MHz, DMSO- d₆) δ 9.41 (s, 1H), 8.62 (s, 1H), 8.03 (d, J = 2.5 Hz, 1H), 7.50-7.42 (m, 1H), 7.38 (tdd, J = 7.5, 5.3, 1.7 Hz, 1H), 7.19 (dd, J = 10.3, 8.0 Hz, 2H), 6.76 (d, J = 2.5 Hz, 1H), 2.54 (s, 6H), 1.59 (q, J = |
| | | | | | | 4.3 Hz, 2H), 1.14 (q, J = 4.3 |
| | | | | | | Hz, 2H). |
| 340 | 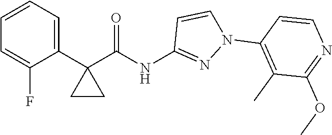 [SMILES: COc1nccc(-n2ccc(NC(=O)C3(c4ccccc4F)CC3)n2)c1C] | B | 5 | >5 | 367.31 | 1H NMR (400 MHz, DMSO- d₆) δ 9.44 (s, 1H), 8.05 (dd, J = 8.2, 4.0 Hz, 2H), 7.46 (td, J = 7.8, 1.8 Hz, 1H), 7.38 (tdd, J = 7.6, 5.3, 1.8 Hz, 1H), 7.27- 7.12 (m, 2H), 7.03 (d, J = 5.5 Hz, 1H), 6.76 (d, J = 2.6 Hz, 1H), 3.91 (s, 3H), 2.14 (s, 3H), 1.59 (q, J = 4.3 Hz, 2H), |
| | | | | | | 1.14 (q, J = 4.3 Hz, 2H). |
| 341 | 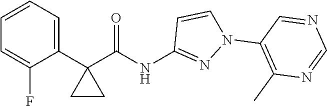 [SMILES: Cc1ncncc1-n1ccc(NC(=O)C2(c3ccccc3F)CC2)n1] | B | 8 | 0.70 | 338.31 | 1H NMR (400 MHz, DMSO- d₆) δ 9.44 (s, 1H), 9.05 (s, 1H), 8.77 (s, 1H), 8.10 (d, J = 2.6 Hz, 1H), 7.47 (td, J = 7.8, 1.8 Hz, 1H), 7.42-7.32 (m, 1H), 7.25-7.14 (m, 2H), 6.79 (d, J = 2.5 Hz, 1H), 2.49 |
| | | | | | | (s, 5H), 1.59 (q, J = 4.3 Hz, |
| | | | | | | 2H), 1.15 (q, J = 4.3 Hz, 2H). |
| 342 | 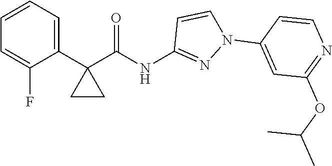 [SMILES: CC(C)Oc1cc(-n2ccc(NC(=O)C3(c4ccccc4F)CC3)n2)ccn1] | B | 6 | >5 | 381.40 | 1H NMR (400 MHz, DMSO- d₆) δ 9.52 (s, 1H), 8.55 (d, J = 2.7 Hz, 1H), 8.14 (d, J = 5.7 Hz, 1H), 7.47 (td, J = 7.8, 1.8 Hz, 1H), 7.40 (tdd, J = 7.5, 5.3, 1.7 Hz, 1H), 7.33 (dd, J = 5.7, 1.9 Hz, 1H), 7.26-7.14 (m, 2H), 7.09 (d, J = 1.9 Hz, 1H), 6.79 (d, J = 2.7 Hz, 1H), 5.26 (h, J = 6.1 Hz, 1H), 1.61 (q, J = 4.3 Hz, |
| | | | | | | 2H), 1.29 (d, J = 6.2 Hz, 6H), |
| | | | | | | 1.16 (q, J = 4.3 Hz, 2H). |
| 343 | 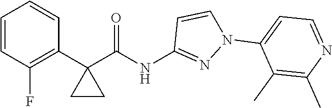 [SMILES: Cc1nccc(-n2ccc(NC(=O)C3(c4ccccc4F)CC3)n2)c1C] | B | 7 | >5 | 351.19 | 1H NMR (400 MHz, DMSO- d₆) δ 9.57 (s, 1H), 8.19 (d, J = 2.6 Hz, 1H), 7.63 (s, 1H), 7.51-7.32 (m, 2H), 7.30- 7.10 (m, 3H), 6.85 (d, J = 2.6 Hz, 1H), 2.53 (s, 6H), 1.61 (m, 2H), 1.17 (m, 2H). |
| 344 | 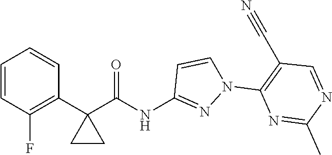 [SMILES: Cc1ncc(C#N)c(-n2ccc(NC(=O)C3(c4ccccc4F)CC3)n2)n1] | B | 3 | >5 | 363.17 | 1H NMR(400 MHz, DMSO- d₆) δ 9.53 (s, 1H), 9.14 (s, 1H), 8.64 (d, J = 2.9 Hz, 1H), 7.52-7.34 (m, 2H), 7.28- 7.18 (m, 2H), 6.93 (d, J = 2.9 Hz, 1H), 2.69 (s, 3H), 1.63 (m, 2H), 1.21 (m, 2H). |
| 345 | 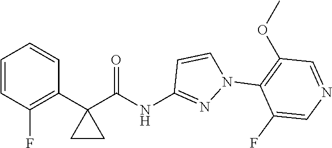 [SMILES: COc1cncc(F)c1-n1ccc(NC(=O)C2(c3ccccc3F)CC2)n1] | B | 7 | >2 | 371.18 | 1H NMR (400 MHz, DMSO- d₆) δ 9.46 (s, 1H), 8.47 (m, 2H), 7.92 (d, J = 2.6 Hz, 1H), 7.51-7.33 (m, 2H), 7.23- 7.13 (m, 2H), 6.75 (d, J = 2.6 Hz, 1H), 3.93 (s, 3H), 1.59 (m, 2H), 1.14 (m, 2H). |
| 346 | 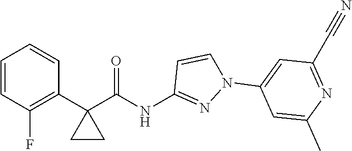 [SMILES: Cc1cc(-n2ccc(NC(=O)C3(c4ccccc4F)CC3)n2)cc(C#N)n1] | B | 36 | >5 | 362.20 | 1H NMR (400 MHz, DMSO- d₆) δ 9.55 (s, 1H), 8.63 (d, J = 2.8 Hz, 1H), 8.22 (d, J = 2.0 Hz, 1H), 7.91 (d, J = 2.0 Hz, 1H), 7.54-7.33 (m, 2H), 7.24-7.16 (m, 2H), 6.89 (d, J = 2.8 Hz, 1H), 2.54 (s, 3H), 1.61 (m, 2H), 1.18 (m, 2H). |
| 347 | 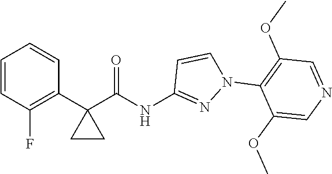 [SMILES: COc1cncc(OC)c1-n1ccc(NC(=O)C2(c3ccccc3F)CC2)n1] | B | 5 | >5 | 383.17 | 1H NMR (400 MHz, DMSO- d₆) δ 9.25 (s, 1H), 8.25 (s, 2H), 7.67 (d, J = 2.5 Hz, 1H), 7.51-7.32 (m, 2H), 7.26- 7.13 (m, 2H), 6.64 (d, J = 2.5 Hz, 1H), 3.82 (s, 6H), 1.58 (m, 2H), 1.13 (m, 2H). |
| 348 | 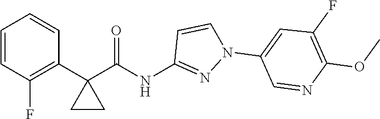 [SMILES: COc1ncc(-n2ccc(NC(=O)C3(c4ccccc4F)CC3)n2)cc1F] | B | 7 | 0.033 | 371.27 | 1H NMR (400 MHz, DMSO- d₆) δ 9.44 (s, 1H), 8.37 (m, 2H), 8.13 (m, 1H), 7.51- 7.32 (m, 2H), 7.26-7.15 (m, 2H), 6.76 (d, J = 2.6 Hz, 1H), 3.96 (s, 3H), 1.60 (m, 2H), 1.15 (m, 2H). |
| 349 | 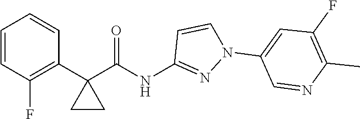 [SMILES: Cc1ncc(-n2ccc(NC(=O)C3(c4ccccc4F)CC3)n2)cc1F] | B | 10 | 0.44 | 355.16 | 1H NMR (400 MHz, DMSO- d₆) δ 9.52 (s, 1H), 8.77 (d, J = 2.1 Hz, 1H), 8.47 (d, J = 2.7 Hz, 1H), 8.04 (m, 1H), 7.54-7.33 (m, 2H), 7.30- 7.15 (m, 2H), 6.80 (d, J = 2.6 Hz, 1H), 2.44 (d, J = 2.7 Hz, 3H), 1.60 (m, 2H), 1.16 (m, |
| | | | | | | 2H). |
| 350 | 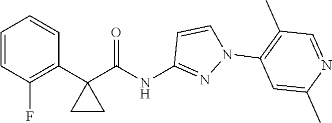 [SMILES: Cc1cc(-n2ccc(NC(=O)C3(c4ccccc4F)CC3)n2)c(C)cn1] | B | 17 | >5 | 351.19 | 1H NMR (400 MHz, DMSO- d₆) δ 9.39 (s, 1H), 8.39 (s, 1H), 8.13 (d, J = 2.6 Hz, 1H), 7.55-7.34 (m, 2H), 7.31 (s, 1H), 7.24-7.14 (m, 2H), 6.77 (d, J = 2.6 Hz, 1H), 2.44 (s, 3H), 2.32 (s, 3H), 1.60 (m, 2H), 1.15 (m, 2H). |
| 351 | 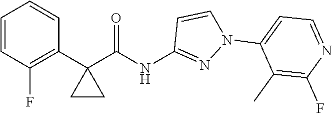 [SMILES: Cc1c(-n2ccc(NC(=O)C3(c4ccccc4F)CC3)n2)ccnc1F] | B | 20 | 0.42 | 355.16 | 1H NMR (400 MHz, DMSO- d₆) δ 9.64 (s, 1H), 8.35- 8.24 (m, 2H), 7.63 (t, J = 5.7 Hz, 1H), 7.50-7.36 (m, 3H), 7.27-7.18 (m, 3H), 6.87 (d, J = 2.8 Hz, 1H), 2.51 (s, 3H), 1.62 (m, 2H), 1.17 (m, 2H). |
| 352 | 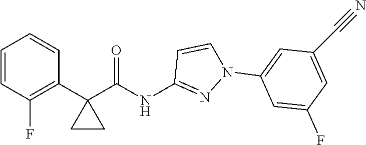 [SMILES: N#Cc1cc(F)cc(-n2ccc(NC(=O)C3(c4ccccc4F)CC3)n2)c1] | B | 8 | >5 | 365.16 | 1H NMR (400 MHz, DMSO- d₆) δ 9.49 (s, 1H), 8.54 (d, J = 2.7 Hz, 1H), 8.11 (d, J = 1.6 Hz, 1H), 7.97 (dt, J = 10.5, 2.3 Hz, 1H), 7.73 (ddd, J = 8.2, 2.4, 1.2 Hz, 1H), 7.47 (td, J = 7.9, 1.9 Hz, 1H), 7.40 (tdd, J = 7.6, 5.3, 1.8 Hz, 1H), 7.26-7.15 (m, 2H), 6.82 (d, J = 2.7 Hz, 1H), 1.60 |
| | | | | | | (q, J = 4.3 Hz, 2H), 1.16 (q, J = |
| | | | | | | 4.3 Hz, 2H). |
| 353 | 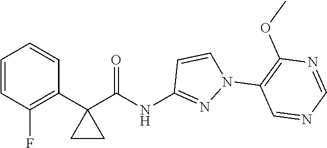 [SMILES: COc1ncncc1-n1ccc(NC(=O)C2(c3ccccc3F)CC2)n1] | B | 3 | 2.2 | 354.18 | 1H NMR (400 MHz, DMSO- d₆) δ 9.51 (s, 1H), 8.80 (s, 1H), 8.72 (s, 1H), 8.20 (d, J = 2.7 Hz, 1H), 7.46 (td, J = 7.8, 1.9 Hz, 1H), 7.39 (tdd, J = 7.3, 5.2, 1.7 Hz, 1H), 7.23- 7.17 (m, 2H), 6.78 (d, J = 2.7 Hz, 1H), 4.05 (s, 3H), 1.59 (q, J = 4.3 Hz, 2H), 1.15 (q, J = 4.3 Hz, 2H). |
| 354 | 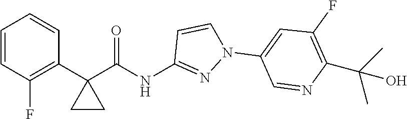 [SMILES: CC(C)(O)c1ncc(-n2ccc(NC(=O)C3(c4ccccc4F)CC3)n2)cc1F] | B | 21 | >5 | 399.32 | 1H NMR (400 MHz, DMSO- d₆) δ 9.57 (s, 1H), 8.78 (dd, J = 2.2, 1.1 Hz, 1H), 8.50 (d, J = 2.7 Hz, 1H), 8.05 (m, 1H), 7.51-7.34 (m, 2H), 7.24- 7.17 (m, 2H), 6.82 (d, J = 2.7 Hz, 1H), 5.26 (s, 1H), 1.61 (m, 2H), 1.51 (d, J = 1.1 Hz, |
| | | | | | | 6H), 1.16 (m, 2H). |
| 355 | 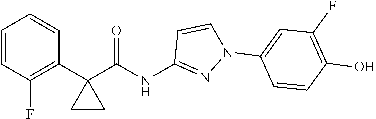 [SMILES: O=C(Nc1ccn(-c2ccc(O)c(F)c2)n1)C1(c2ccccc2F)CC1] | B | 31 | 1.7 | 356.28 | 1H NMR (400 MHz, DMSO- d₆) δ 9.99 (s, 1H), 9.27 (s, 1H), 8.24 (d, J = 2.6 Hz, 1H), 7.54 (dd, J = 12.5, 2.6 Hz, 1H), 7.50-7.44 (m, 1H), 7.44-7.32 (m, 2H), 7.26- 7.16 (m, 2H), 6.98 (t, J = 9.1 Hz, 1H), 6.67 (d, J = 2.6 Hz, |
| | | | | | | 1H), 1.58 (q, J = 4.2 Hz, 2H), |
| | | | | | | 1.14 (q, J = 4.3 Hz, 2H). |
| 356 | 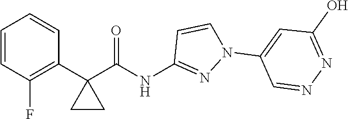 [SMILES: O=C(Nc1ccn(-c2cnnc(O)c2)n1)C1(c2ccccc2F)CC1] | B | 5 | >5 | 340.19 | 1H NMR (400 MHz, DMSO- d₆) δ 13.10 (s, 1H), 9.76 (s, 1H), 8.57 (d, J = 2.8 Hz, 1H), 8.46 (d, J = 2.5 Hz, 1H), 7.45 (td, J = 7.8, 1.9 Hz, 1H), 7.39 (tdd, J = 7.5, 5.3, 1.7 Hz, 1H), 7.25-7.10 (m, 2H), 7.01 (t, J = 2.1 Hz, 1H), 6.88 |
| | | | | | | (d, J = 2.8 Hz, 1H), 1.61 (q, J = |
| | | | | | | 4.3 Hz, 2H), 1.16 (q, J = |
| | | | | | | 4.4 Hz, 2H). |
| 357 | 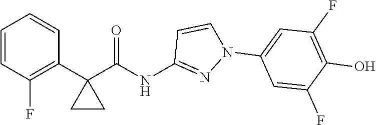 [SMILES: O=C(Nc1ccn(-c2cc(F)c(O)c(F)c2)n1)C1(c2ccccc2F)CC1] | B | 21 | >5 | 374.29 | 1H NMR (400 MHz, DMSO- d₆) δ 10.19 (s, 1H), 9.27 (s, 1H), 8.25 (d, J = 2.6 Hz, 1H), 7.45-7.36 (m, 2H), 7.36- 7.28 (m, 1H), 7.18-7.09 (m, 2H), 6.63 (d, J = 2.5 Hz, 1H), 1.52 (q, J = 4.2 Hz, 2H), 1.07 (q, J = 4.3 Hz, 2H). |
| 374 | 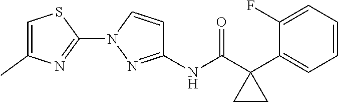 [SMILES: Cc1csc(-n2ccc(NC(=O)C3(c4ccccc4F)CC3)n2)n1] | D | 8 | 0.47 | 343.15 | 1H NMR (300 MHz, CDCl₃) δ 8.15 (d, J = 2.7, 1H), 7.71 (s, 1H), 7.50-7.41 (m, 2H), 7.22-7.12 (m, 1H), 7.02 (d, J = 2.7, 1H), 6.58 (s, 1H), 2.39 (s, 3H), 1.81 (m, 2H), 1.21 (m, 2H). |
| 375 | 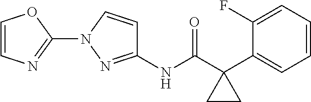 [SMILES: O=C(Nc1ccn(-c2ncco2)n1)C1(c2ccccc2F)CC1] | D | 33 | 0.50 | 312.92 | 1H NMR (300 MHz, CDCl₃) δ 8.06 (d, J = 2.8 Hz, 1H), 7.54 (d, J = 0.9 Hz, 1H), 7.49- 7.36 (m, 2H), 7.25-7.08 (m, 4H), 1.82 (m, J = 4.1 Hz, 2H), 1.24 (m, J = 4.1 Hz, 2H). |
| 381 | 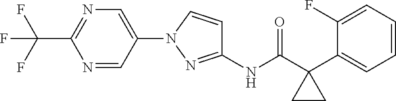 [SMILES: O=C(Nc1ccn(-c2cnc(C(F)(F)F)nc2)n1)C1(c2ccccc2F)CC1] | B | 18 | >5 | 392.21 | 1H NMR (400 MHz, DMSO- d₆) δ 9.78 (s, 1H), 9.40 (s, 2H), 8.67 (d, J = 2.7 Hz, 1H), 7.55-7.45 (m, 1H), 7.40 (d, J = 7.5 Hz, 1H), 7.25-7.18 (m, 2H), 6.94 (d, J = 2.7 Hz, 1H), 1.62 (q, J = 4.3 Hz, 2H), |
| | | | | | | 1.18 (q, J = 4.3 Hz, 2H). |
| 382 | 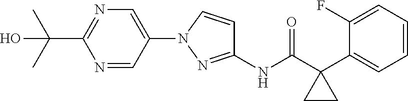 [SMILES: CC(C)(O)c1ncc(-n2ccc(NC(=O)C3(c4ccccc4F)CC3)n2)cn1] | B | 37 | >5 | 382.21 | 1H NMR (400 MHz, DMSO- d₆) δ 9.63 (s, 1H), 9.16 (s, 2H), 8.52 (d, J = 2.6 Hz, 1H), 7.52-7.44 (m, 1H), 7.43- 7.36 (m, 1H), 7.28-7.16 (m, 2H), 6.85 (d, J = 2.6 Hz, 1H), 5.15 (s, 1H), 1.61 (q, J = 4.3 |
| | | | | | | Hz, 2H), 1.51 (s, 6H), 1.16 |
| | | | | | | (q, J = 4.3 Hz, 2H). |
| 383 | 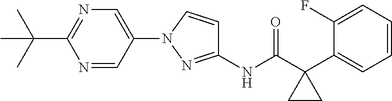 [SMILES: CC(C)(C)c1ncc(-n2ccc(NC(=O)C3(c4ccccc4F)CC3)n2)cn1] | B | 27 | >5 | 380.28 | 1H NMR (400 MHz, DMSO- d₆) δ 9.62 (s, 1H), 9.11 (s, 2H), 8.48 (d, J = 2.6 Hz, 1H), 7.56-7.32 (m, 2H), 7.26- 7.17 (m, 2H), 6.83 (d, J = 2.6 Hz, 1H), 1.61 (q, J = 4.3 Hz, 2H), 1.36 (s, 9H), 1.16 (q, J = 4.3 Hz, 2H). |
| 384 | 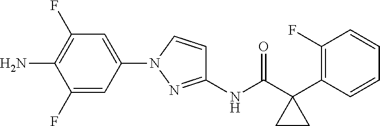 [SMILES: Nc1c(F)cc(-n2ccc(NC(=O)C3(c4ccccc4F)CC3)n2)cc1F] | B | 20 | 0.98 | 373.21 | 1H NMR (400 MHz, DMSO- d₆) δ 9.26 (s, 1H), 8.24 (d, J = 2.5 Hz, 1H), 7.47 (t, J = 7.7 Hz, 1H), 7.45-7.32 (m, 2H), 7.28-7.17 (m, 2H), 6.66 (d, J = 2.5 Hz, 1H), 5.76 (s, 1H), 5.25 (s, 2H), 1.59 (q, J = 4.3 Hz, 2H), 1.14 (q, J = 4.2 Hz, |
| | | | | | | 2H). |
| 385 | 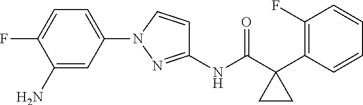 [SMILES: Nc1cc(-n2ccc(NC(=O)C3(c4ccccc4F)CC3)n2)ccc1F] | B | 20 | 1.4 | 355.24 | 1H NMR (400 MHz, DMSO- d₆) δ 9.13 (s, 1H), 8.16 (d, J = 2.5 Hz, 1H), 7.49 (t, J = 7.6 Hz, 1H), 7.46-7.37 (m, 1H), 7.23 (t, J = 8.2 Hz, 2H), 7.10 (dd, J = 8.0, 2.7 Hz, 1H), 7.03 (dd, J = 11.0, 8.8 Hz, |
| | | | | | | 1H), 6.80 (dt, J = 8.7, 3.4 Hz, |
| | | | | | | 1H), 6.66 (d, J = 2.5 Hz, 1H), |
| | | | | | | 5.76 (s, 1H), 5.39 (s, 2H), |
| | | | | | | 1.59 (q, J = 4.2 Hz, 2H), 1.15 |
| | | | | | | (q, J = 4.2 Hz, 2H). |
| 387 | 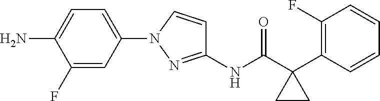 [SMILES: Nc1ccc(-n2ccc(NC(=O)C3(c4ccccc4F)CC3)n2)cc1F] | B | 15 | 1.1 | 355.12 | 1H NMR (400 MHz, DMSO- d₆) δ 9.18 (s, 1H), 8.15 (d, J = 2.5 Hz, 1H), 7.54-7.45 (m, 1H), 7.45-7.36 (m, 2H), 7.28-7.18 (m, 2H), 6.79 (t, J = 9.2 Hz, 1H), 6.63 (d, J = 2.4 Hz, 1H), 5.76 (s, 1H), |
| | | | | | | 5.21 (s, 2H), 1.58 (q, J = 4.2 |
| | | | | | | Hz, 2H), 1.14 (q, J = 4.3 Hz, |
| | | | | | | 2H). |
| 388 | 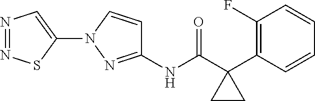 [SMILES: O=C(Nc1ccn(-c2cnns2)n1)C1(c2ccccc2F)CC1] | B | 2 | 0.037 | 330.16 | 1H NMR (400 MHz, DMSO- d₆) δ 9.96 (s, 1H), 9.25 (s, 1H), 8.54 (d, J = 2.7 Hz, 1H), 7.53-7.32 (m, 2H), 7.23- 7.14 (m, 2H), 6.92 (d, J = 2.7 Hz, 1H), 1.61 (q, J = 4.3 Hz, 2H), 1.17 (q, J = 4.4 Hz, 2H). |
| 401 | 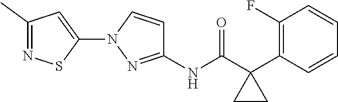 [SMILES: Cc1cc(-n2ccc(NC(=O)C3(c4ccccc4F)CC3)n2)sn1] | B | 3 | 2.3 | 343.05 | 1H NMR (400 MHz, DMSO- d₆) δ 9.79 (s, 1H), 8.38 (d, J = 2.7 Hz, 1H), 7.49-7.35 (m, 3H), 7.24-7.14 (m, 2H), 6.83 (d, J = 2.7 Hz, 1H), 1.60 (q, J = 4.4 Hz, 2H), 1.15 (q, J = 4.4 Hz, 2H). |
| 402 | 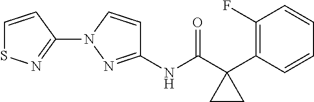 [SMILES: O=C(Nc1ccn(-c2ccsn2)n1)C1(c2ccccc2F)CC1] | B | 3 | 0.060 | 329.05 | 1H NMR (400 MHz, DMSO- d₆) δ 9.53 (s, 1H), 9.14 (d, J = 4.8 Hz, 1H), 8.36 (d, J = 2.6 Hz, 1H), 7.52-7.43 (m, 2H), 7.45-7.34 (m, 1H), 7.26-7.16 (m, 2H), 6.77 (d, J = 2.7 Hz, 1H), 1.61 (q, J = |
| | | | | | | 4.3 Hz, 2H), 1.16 (q, J = 4.4 |
| | | | | | | Hz, 2H). |
| 403 | 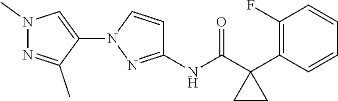 [SMILES: Cc1nn(C)cc1-n1ccc(NC(=O)C2(c3ccccc3F)CC2)n1] | B | 3 | >5 | 340.10 | 1H NMR (400 MHz, DMSO- d₆) δ 9.11 (s, 1H), 7.89 (s, 1H), 7.77 (d, J = 2.4 Hz, 1H), 7.47 (td, J = 7.6, 1.8 Hz, 1H), 7.39 (tdd, J = 7.5, 5.3, 1.8 Hz, 1H), 7.21 (t, J = 8.9 Hz, 2H), 6.60 (d, J = 2.4 Hz, 1H), |
| | | | | | | 3.75 (s, 3H), 2.15 (s, 3H), |
| | | | | | | 1.57 (q, J = 4.2 Hz, 2H), 1.13 |
| | | | | | | (q, J = 4.3 Hz, 2H). |
| 404 | 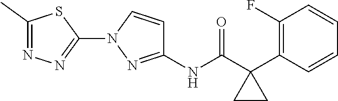 [SMILES: Cc1nnc(-n2ccc(NC(=O)C3(c4ccccc4F)CC3)n2)s1] | B | 3 | >5 | 343.95 | 1H NMR (400 MHz, DMSO- d₆) δ 9.94 (s, 1H), 8.46 (d, J = 2.8 Hz, 1H), 7.49-7.33 (m, 2H), 7.24-7.14 (m, 2H), 6.87 (d, J = 2.8 Hz, 1H), 2.68 (s, 3H), 1.61 (q, J = 4.4 Hz, |
| | | | | | | 2H), 1.17 (q, J = 4.4 Hz, 2H). |
| 405 | 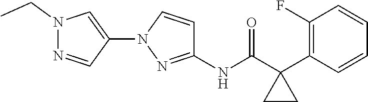 [SMILES: CCn1cc(-n2ccc(NC(=O)C3(c4ccccc4F)CC3)n2)cn1] | B | 5 | RND | 340.20 | 1H NMR (400 MHz, DMSO- d₆) δ 9.12 (s, 1H), 8.07 (s, 1H), 7.98 (d, J = 2.4 Hz, 1H), 7.71 (s, 1H), 7.48 (td, J = 7.6, 1.8 Hz, 1H), 7.46-7.35 (m, 1H), 7.22 (t, J = 8.3 Hz, 2H), 6.60 (d, J = 2.4 Hz, 1H), |
| | | | | | | 4.11 (q, J = 7.3 Hz, 2H), 1.58 |
| | | | | | | (q, J = 4.2 Hz, 2H), 1.36 (t, |
| | | | | | | J = 7.3 Hz, 3H), 1.13 (q, J = |
| | | | | | | 4.3 Hz, 2H). |
| 406 | 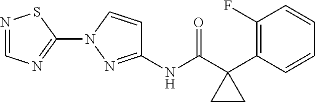 [SMILES: O=C(Nc1ccn(-c2ncns2)n1)C1(c2ccccc2F)CC1] | B | 11 | 0.018 | 330.02 | 1H NMR (400 MHz, DMSO- d₆) δ 10.11 (s, 1H), 8.62 (s, 1H), 8.47 (d, J = 2.9 Hz, 1H), 7.49-7.33 (m, 2H), 7.24- 7.14 (m, 2H), 6.96 (d, J = 2.9 Hz, 1H), 2.55 (s, 4H), 1.63 (q, J = 4.4 Hz, 2H), 1.18 (q, |
| | | | | | | J = 4.4 Hz, 2H). |
| 432 | 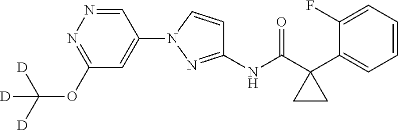 [SMILES: [2H]C([2H])([2H])Oc1cc(-n2ccc(NC(=O)C3(c4ccccc4F)CC3)n2)cnn1] | E | 3 | RND | 357.12 | 1H NMR (400 MHz, Methanol-d₄) δ 9.33 (s, 1H), 8.42 (d, J = 2.9 Hz, 1H), 7.55- 7.44 (m, 1H), 7.48-7.39 (m, 2H), 7.26 (td, J = 7.6, 1.2 Hz, 1H), 7.18 (ddd, J = 10.4, 8.3, 1.2 Hz, 1H), 6.97 (d, J = 2.9 Hz, 1H), 1.70 (q, J = 4.2 |
| | | | | | | Hz, 2H), 1.28-1.20 (m, 2H). |

*The Average HEK293 IC50 reflects the average of one or more replicates.
RND = not determined yet

### Example 1.5. Compounds Prepared Using SnAr as Final Step

### Scheme S_{N}Ar-1. Preparation of Compounds Listed in Table C

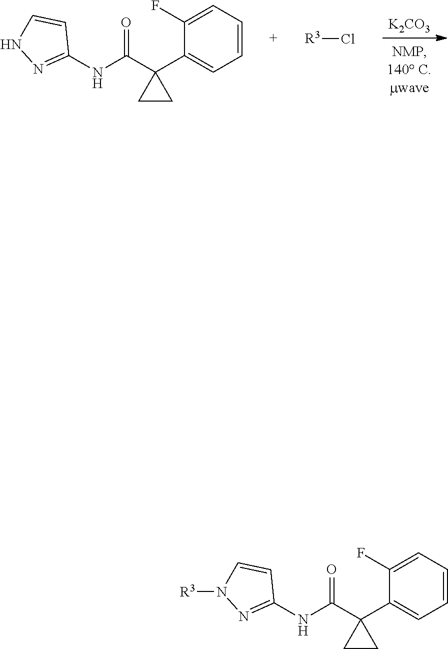

Scheme S_{N}Ar-1 provides a general synthetic route for the preparation of compounds listed in Table C. Using 1-(2-fluorophenyl)-N-(1H-pyrrol-3-yl)cyclopropane-1-carboxamide and the appropriate selection of aryl halide, compounds were synthesized according to the representative procedure described below for 1-(2-fluorophenyl)-N-(1-(6-methoxypyrimidin-4-yl)-1H-pyrazol-3-yl)cyclopropane-1-carboxamide. The reaction yield and characterization information for each compound is listed within Table C.

### Representative Procedure for SnAr Reaction

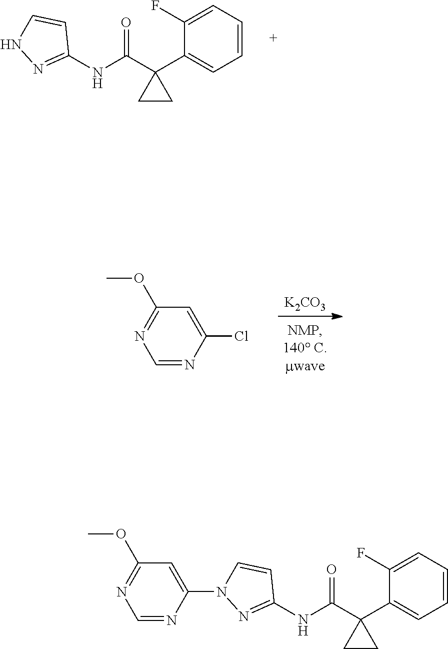

### 1-(2-fluorophenyl)-N-(1-(6-methoxypyrimidin-4-yl)-1H-pyrazol-3-yl)cyclopropane-1-carboxamide (Compound 358)

1-(2-fluorophenyl)-N-(1H-pyrazol-3-yl)cyclopropanecarboxamide (30 mg, 0.12 mmol) was dissolved in N-methylpyrrolidin-2-one (3.0 mL). To the resultant solution was added potassium carbonate (35 mg, 0.25 mmol) and 4-chloro-6-methoxy-pyrimidine (20 mg, 0.14 mmol). The reaction vessel was sealed and heated in a microwave at 140° C. for 30 minutes. The reaction mixture was cooled to room temperature, diluted with dichloromethane, and washed with 1N NaOH and saturated aqueous NaCl. The organics were concentrated, and the crude residue was purified by C18 preparatory HPLC (acetonitrile/water with TFA modifier) to provide 1-(2-fluorophenyl)-N-[1-(6-methoxypyrimidin-4-yl)pyrazol-3-yl]cyclopropanecarboxamide (Compound 358, 3.4 mg, 6%).

TABLE C
Compounds Prepared Using SnAr as Final Step

| | | | Average | | |
|---|---|---|---|---|---|
| | | | HEK293 | | |
| Cpd | | Yield | IC50 | | NMR |
| No. | Structure | (%) | (uM)* | M + 1 | (shifts in ppm) |
| 358 | 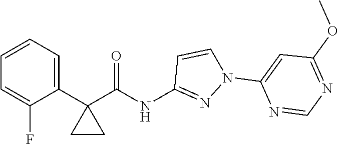 [SMILES: COc1cc(-n2ccc(NC(=O)C3(c4ccccc4F)CC3)n2)ncn1] | 6 | >5 | 354.354 | 1H NMR (300 MHz, CDCl₃) δ 8.57 (d, J = 1.0 Hz, 1H), 8.39 (d, J = 2.8 Hz, 1H), 7.72 (s, 1H), 7.45 (m, 2H), 7.24- 7.13 (m, 1H), 7.02 (d, J = 2.8 Hz, 1H), 6.94 (d, J = 1.0 Hz, 1H), 4.00 (s, 3H), 1.82 (m, 2H), 1.23 |
| | | | | | (m, 2H). |
| 359 | 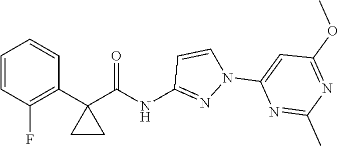 [SMILES: COc1cc(-n2ccc(NC(=O)C3(c4ccccc4F)CC3)n2)nc(C)n1] | 7 | >5 | 368.33 | 1H NMR (300 MHz, CDCl₃) δ 8.41 (dd, J = 2.8, 0.5 Hz, 1H), 7.52- 7.39 (m, 2H), 7.25- 7.13 (m, 2H), 7.00 (d, J = 2.8 Hz, 1H), 6.75 (d, J = 0.7 Hz, 1H), 3.97 (s, 3H), 2.59 (s, 3H), 1.82 (m, 2H), 1.22 (m, 2H). |
| 360 | 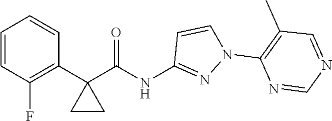 [SMILES: Cc1cncnc1-n1ccc(NC(=O)C2(c3ccccc3F)CC2)n1] | 21 | 0.069 | 338.30 | 1H NMR (300 MHz, CDCl₃) δ 8.85 (s, 1H), 8.44 (m, 1H), 7.53- 7.39 (m, 2H), 7.26- 7.10 (m, 2H), 7.04 (d, J = 2.8 Hz, 1H), 2.53 (s, 3H), 1.83 (m, 2H), 1.23 (m, 2H). |
| 361 | 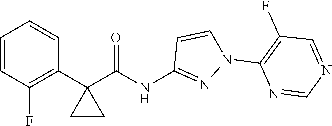 [SMILES: O=C(Nc1ccn(-c2ncncc2F)n1)C1(c2ccccc2F)CC1] | 29 | 0.15 | 342.30 | 1H NMR (300 MHz, CDCl₃) δ 8.85 (d, J = 2.4 Hz, 1H), 8.66 (d, J = 4.3 Hz, 1H), 8.32 (m, 1H), 7.96 (s, 1H), 7.48- 7.36 (m, 2H), 7.27- 7.10 (m, 3H), 1.88- 1.77 (m, 2H), 1.30- |
| | | | | | 1.17 (m, 2H). |

*The Average HEK293 IC50 reflects the average of one or more replicates.

### Example 1.6. Compounds Prepared Using a Boronic Acid Coupling Sequence

### Scheme Boron-1. Preparation of Compounds Listed in Table D

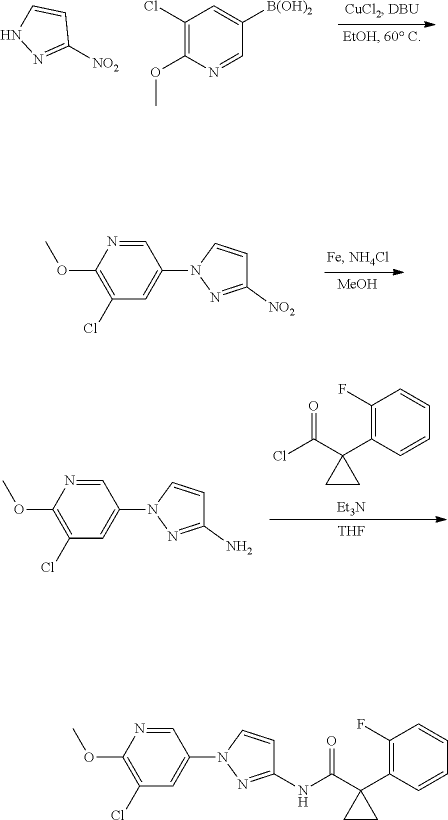

### Step 1: 3-chloro-2-methoxy-5-(3-nitro-1H-pyrazol-1-yl)pyridine

3-nitro-1H-pyrazole (145 mg, 1.28 mmol, 1.2 eq), copper (II) chloride (14.4 mg, 0.11 mmol, 0.1 eq), DBU (199 μL, 1.33 mmol, 1.25 eq), and ethanol (5.0 mL) were combined and stirred for 5 minutes. (5-chloro-6-methoxypyridin-3-yl)boronic acid (200 mg, 1.07 mmol, 1.0 eq) was added, air was bubbled through the reaction, and the mixture was heated to 60° C. for 5 days. The mixture was filtered through celite, and the filtrate evaporated. The crude residue was dissolved in dichlormethane and washed with 2N NaOH, saturated aqueous NH₄Cl, water, and brine. The organic layer was collected and evaporated to provide 3-chloro-2-methoxy-5-(3-nitro-1H-pyrazol-1-yl)pyridine, the full quantity of which was carried forward in the following step without further manipulation.

### Step 2: 1-(5-chloro-6-methoxypyridin-3-yl)-1H-pyrazol-3-amine

3-chloro-2-methoxy-5-(3-nitro-1H-pyrazol-1-yl)pyridine from Step 1 was dissolved in methanol (5.0 mL), to which was added iron (119 mg, 2.13 mmol, 2.0 eq) and 7M NH₄Cl (457 μL, 3.2 mmol, 3.0 eq). The reaction mixture was stirred 16 h, then the crude reaction mixture was filtered through Celite. The filtrate was evaporated to provide 1-(5-chloro-6-methoxypyridin-3-yl)-1H-pyrazol-3-amine, the full quantity of which was carried forward in the following step without further manipulation.

### Step 3: N-(1-(5-chloro-6-methoxypyridin-3-yl)-1H-pyrazol-3-yl)-1-(2-fluorophenyl)cyclopropane-1-carboxamide (Compound 378)

1-(5-chloro-6-methoxypyridin-3-yl)-1H-pyrazol-3-amine from Step 2 was dissolved in tetrahydrofuran (5.0 mL). To the solution was added triethylamine (297 μL, 2.13 mmol, 2.0 eq) and 1-(2-fluorophenyl)cyclopropane-1-carbonyl chloride (212 mg, 1.07 mmol, 1.0 eq). The resultant mixture was stirred 16 h, and the solvent was then evaporated. The crude residue was dissolved in dichloromethane and washed with saturated aqueous NaHCO₃. The organic layer was collected and evaporated, and the crude residue was purified by C18 preparatory HPLC (acetonitrile/water with ammonium hydroxide modifier) to provide N-(1-(5-chloro-6-methoxypyridin-3-yl)-1H-pyrazol-3-yl)-1-(2-fluorophenyl)cyclopropane-1-carboxamide (16.7 mg, 4% yield).

TABLE D
Compounds prepared using boronic acid coupling sequence

| | | | Average | | |
|---|---|---|---|---|---|
| Cpd | | Yield | HEK293 | | NMR |
| No. | Structure | (%) | IC50 (uM)* | M + 1 | (shifts in ppm) |
| 376 | 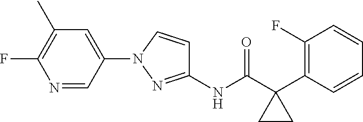 [SMILES: Cc1cc(-n2ccc(NC(=O)C3(c4ccccc4F)CC3)n2)cnc1F] | 4 | 0.065 | 355.17 | 1H NMR (300 MHz, CDCl₃) δ 8.21 (ddd, J = 2.5, 1.7, 0.8 Hz, 1H), 7.82 (ddd, J = 8.3, 2.7, 0.9 Hz, 1H), 7.75 (dd, J = 2.6, 0.5 Hz, 1H), 7.51-7.38 (m, 2H), 7.26-7.13 (m, 2H), 7.01 (d, J = 2.5 Hz, 1H), 2.33 (dt, J = 1.4, 0.7 Hz, 3H), 1.82 (m, |
| | | | | | 2H), 1.27-1.18 (m, 2H). |
| 377 | 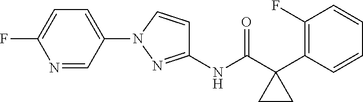 [SMILES: O=C(Nc1ccn(-c2ccc(F)nc2)n1)C1(c2ccccc2F)CC1] | 2 | 0.023 | 341.20 | 1H NMR (300 MHz, CDCl₃) δ 8.42 (ddd, J = 2.9, 1.5, 0.6 Hz, 1H), 7.98 (ddd, J = 8.8, 6.7, 2.9 Hz, 1H), 7.77 (dd, J = 2.6, 0.5 Hz, 1H), 7.74 (s, 1H), 7.51- 7.38 (m, 2H), 7.27-7.14 (m, |
| | | | | | 2H), 7.03 (d, J = 2.6 Hz, 1H), |
| | | | | | 7.02-6.98 (m, 1H), 1.82 (m, |
| | | | | | 2H), 1.23 (m, 2H). |
| 378 | 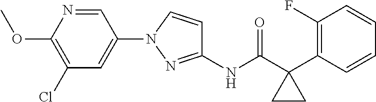 [SMILES: COc1ncc(-n2ccc(NC(=O)C3(c4ccccc4F)CC3)n2)cc1Cl] | 4 | 0.12 | 387.15 | 1H NMR (400 MHz, DMSO-d₆) δ 9.43 (s, 1H), 8.52 (d, J = 2.5 Hz, 1H), 8.37 (d, J = 2.6 Hz, 1H), 8.30 (d, J = 2.5 Hz, 1H), 7.47 (td, J = 7.9, 1.9 Hz, 1H), 7.44-7.34 (m, 1H), 7.29- 7.14 (m, 2H), 6.75 (d, J = 2.6 |
| | | | | | Hz, 1H), 3.95 (s, 3H), 1.59 (q, J = |
| | | | | | 4.3 Hz, 2H), 1.15 (q, J = 4.3 |
| | | | | | Hz, 2H). |
| 379 | 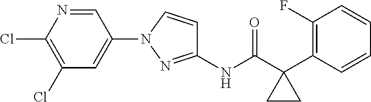 [SMILES: O=C(Nc1ccn(-c2cnc(Cl)c(Cl)c2)n1)C1(c2ccccc2F)CC1] | 2 | 0.12 | 391.10 | 1H NMR (400 MHz, DMSO-d₆) δ 9.58 (s, 1H), 8.82 (d, J = 2.5 Hz, 1H), 8.54 (d, J = 2.7 Hz, 1H), 8.51 (d, J = 2.5 Hz, 1H), 7.46 (td, J = 7.8, 1.8 Hz, 1H), 7.43-7.33 (m, 1H), 7.29- 7.16 (m, 2H), 6.83 (d, J = 2.7 |
| | | | | | Hz, 1H), 1.60 (q, J = 4.3 Hz, |
| | | | | | 2H), 1.16 (q, J = 4.4 Hz, 2H). |

*The Average HEK293 IC50 reflects the average of one or more replicates.
RND = not determined yet

### Example 1.7. Compounds Prepared Via Miscellaneous Methods

### 2-(2-fluorophenyl)-N-methyl-N-(1-phenyl-1H-pyrazol-3-yl)acetamide (Compound 362)

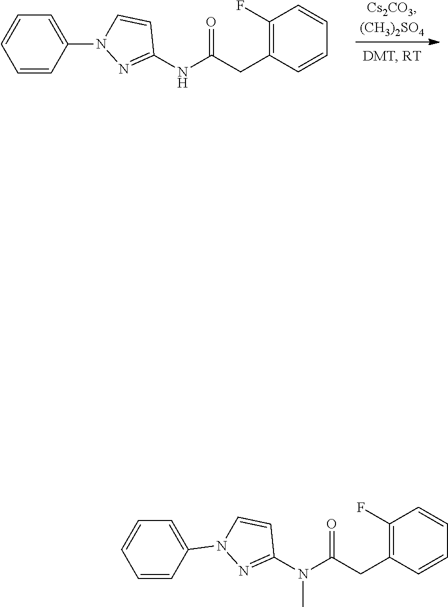

2-(2-Fluorophenyl)-N-(1-phenyl-1H-pyrazol-3-yl)acetamide (63 mg, 0.21 mmol) was dissolved in DMF (1.0 mL). Cesium carbonate (152 mg, 0.47 mmol) and dimethyl sulfate (30 μL, 0.32 mmol) were added, and the resultant reaction mixture was stirred 24 h at room temperature. Additional dimethyl sulfate (20 μL, 0.2114 mmol) was added, and the reaction was stirred a further 6 h. The reaction mixture was partitioned between ethyl acetate and water. The layers were separated, and the organic layer was washed with brine, dried (Na₂SO₄), filtered, and concentrated. The crude oil was purified by silica chromatography (12 g silica column; linear gradient of 0-50% ethyl acetate/heptane) to provide 2-(2-fluorophenyl)-N-methyl-N-(1-phenyl-1H-pyrazol-3-yl)acetamide (46.7 mg, 71% yield) as a white solid. 1H NMR (300 MHz, CDCl₃) δ 7.90 (s, 1H), 7.74-7.57 (m, 2H), 7.49 (m, 2H), 7.43-7.19 (m, 3H), 7.19-6.86 (m, 2H), 6.29 (s, 1H), 3.82 (s, 2H), 3.37 (s, 3H) ppm. ESI-MS m/z calc. 309.13, found 310.49 (M+1).

### 1-(2-fluorophenyl)-N-(1-(2-fluoropyridin-4-yl)-1H-pyrazol-3-yl)-N-methylcyclopropane-1-carboxamide (Compound 363)

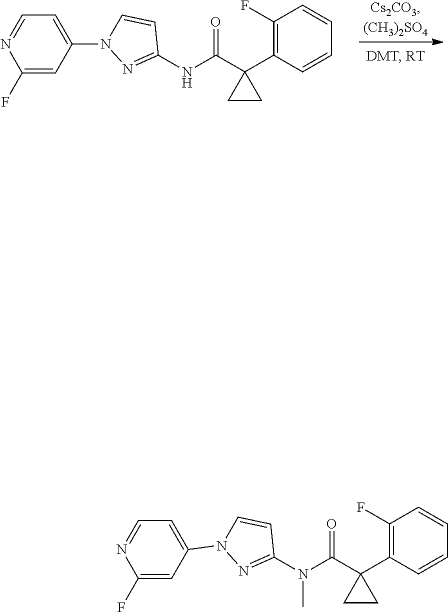

Compound 87 (30 mg, 0.09 mmol) was dissolved in DMF (500 μL). Cesium carbonate (63 mg, 0.19 mmol) and dimethyl sulfate (42 μL, 0.4439 mmol) were added, and the reaction mixture was stirred 48 h at room temperature (˜50% conversion to product observed by LCMS). The reaction mixture was partitioned between ethyl acetate and water. The layers were separated, and the organic layer was washed with brine, dried (Na₂SO₄), filtered, and concentrated. The crude oil was purified by silica chromatography (12 g silica column; linear gradient of 0-50% ethyl acetate/heptane) to provide 1-(2-fluorophenyl)-N-(1-(2-fluoropyridin-4-yl)-1H-pyrazol-3-yl)-N-methylcyclopropane-1-carboxamide (9.2 mg, 28% yield) as a white solid. 1H NMR (300 MHz, CDCl₃) δ 8.23 (dd, J=5.7, 2.2 Hz, 1H), 7.71 (t, J=2.5 Hz, 1H), 7.35 (dt, J=5.7, 1.6 Hz, 1H), 7.19-7.01 (m, 2H), 6.91 (ddd, J=9.4, 8.5, 1.3 Hz, 1H), 6.82 (d, J=7.3 Hz, 2H), 6.44 (s, 1H), 3.31 (d, J=2.2 Hz, 3H), 1.74 (dd, J=4.8, 2.6 Hz, 2H), 1.24-1.12 (m, 2H) ppm. ESI-MS m/z calc. 354.13, found 355.09 (M+1).

### 1-(2-fluorophenyl)-N-(1-(2-hydroxypyridin-4-yl)-1H-pyrazol-3-yl)cyclopropane-1-carboxamide (Compound 364)

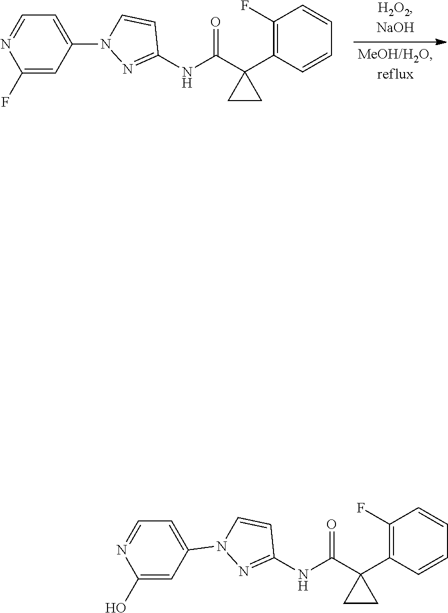

1-(2-Fluorophenyl)-N-(1-(2-fluoropyridin-4-yl)-1H-pyrazol-3-yl)cyclopropane-1-carboxamide (200 mg, 0.59 mmol) was dissolved in methanol (5.4 mL). To the solution was added H₂O₂ (1 mL of 30% w/w, 8.82 mmol) and NaOH (1 mL of 6 M, 6.00 mmol). The resultant mixture was heated to reflux for 72 h. The solvent was reduced, and water was added resulting in precipitation of a white solid, which was collected by vacuum filtration and air-dried. The solid was dissolved in hot methanol, hot-filtered and then cooled first to room temperature followed by cooling to 0° C. The precipitate was collected by vacuum filtration and air-dried to provide 1-(2-fluorophenyl)-N-(1-(2-hydroxypyridin-4-yl)-1H-pyrazol-3-yl)cyclopropane-1-carboxamide (42.5 mg, 20% yield) as a colorless solid. 1H NMR (400 MHz, DMSO-d₆) δ 11.57 (s, 1H), 9.60 (s, 1H), 8.47 (d, J=2.8 Hz, 1H), 7.53-7.32 (m, 3H), 7.28-7.12 (m, 2H), 6.80 (d, J=2.7 Hz, 1H), 6.70 (dd, J=7.2, 2.3 Hz, 1H), 6.60 (d, J=2.2 Hz, 1H), 1.60 (q, J=4.3 Hz, 2H), 1.16 (q, J=4.4 Hz, 2H) ppm. ESI-MS m/z calc. 338.12, found 338.98 (M+1).

### N-(4-fluoro-1-(2-fluoropyridin-4-yl)-1H-pyrazol-3-yl)-1-(2-fluorophenyl)cyclopropane-1-carboxamide (Compound 365)

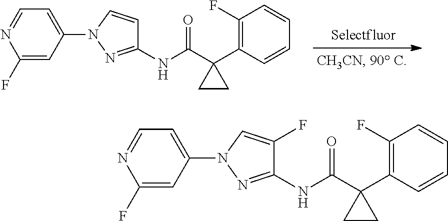

To a solution of 1-(2-fluorophenyl)-N-(1-(2-fluoropyridin-4-yl)-1H-pyrazol-3-yl)cyclopropane-1-carboxamide (20 mg, 0.06 mmol) in acetonitrile (1.0 mL) was added Selectfluor (50 mg, 0.14 mmol). The resultant mixture was stirred at room temperature for 24 h, then 90° C. for 48 h. The solvent was removed, and the crude residue was purified by C18 preparatory HPLC (acetonitrile/water with TFA modifier). The material thus obtained was dissolved in dichloromethane/methanol and passed through a PL-HCO3 MP SPE cartridge. The filtrate was concentrated to provide N-(4-fluoro-1-(2-fluoropyridin-4-yl)-1H-pyrazol-3-yl)-1-(2-fluorophenyl)cyclopropane-1-carboxamide (6.0 mg, 27% yield). 1H NMR (400 MHz, Methanol-d4) δ 8.46 (d, J=4.5 Hz, 1H), 8.20 (d, J=5.8 Hz, 1H), 7.63 (ddd, J=5.8, 1.9, 1.2 Hz, 1H), 7.50 (td, J=7.6, 1.8 Hz, 1H), 7.50-7.35 (m, 2H), 7.28-7.12 (m, 2H), 2.03 (s, 1H), 1.70 (q, J=4.2 Hz, 2H), 1.32-1.19 (m, 2H) ppm. ESI-MS m/z calc. 358.10, found 359.06 (M+1).

### N-[4-fluoro-1-(5-fluoro-3-pyridyl)pyrazol-3-yl]-1-phenyl-cyclopropanecarboxamide (Compound 445)

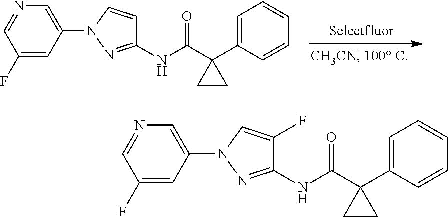

To a solution of Compound 201 (10 mg, 0.031 mmol) in acetonitrile (2.0 mL) was added Selectfluor (20 mg, 0.056 mmol). The resultant reaction mixture was stirred at room temperature for 24 h, then 100° C. for 48 days. The reaction mixture was diluted with DMSO and directly purified by by C18 preparatory HPLC (acetonitrile/water with TFA modifier) to provide N-[4-fluoro-1-(5-fluoro-3-pyridyl)pyrazol-3-yl]-1-phenyl-cyclopropanecarboxamide (Trifluoroacetate salt, 2.4 mg, 15%). 1H NMR (400 MHz, Methanol-d₄) δ 8.81 (dd, J=2.2, 0.9 Hz, 1H), 8.43-8.36 (m, 2H), 8.01 (dt, J=9.9, 2.3 Hz, 1H), 7.56-7.49 (m, 2H), 7.48-7.38 (m, 2H), 7.40-7.31 (m, 1H), 1.63 (q, J=3.9 Hz, 2H), 1.24 (q, J=4.0 Hz, 2H) ppm. ESI-MS m/z calc. 340.11, found 341.07 (M+1).

### N-(1-(5-bromopyrimidin-2-yl)-1H-pyrazol-3-yl)-1-(2-fluorophenyl)cyclopropane-1-carboxamide (Compound 380)

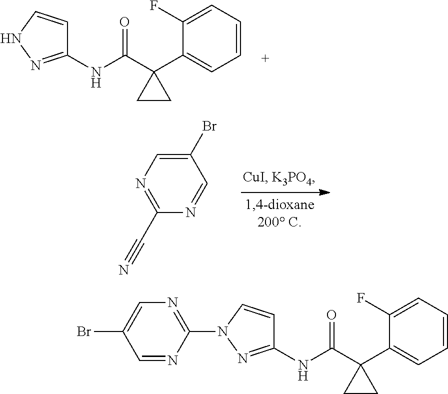

1-(2-fluorophenyl)-N-(1H-pyrazol-3-yl)cyclopropanecarboxamide (63 mg, 0.25 mmol, 1.0 eq), 5-bromopyrimidine-2-carbonitrile (56 mg, 0.30 mol, 1.2 eq), Copper (I) Iodide (40 mg, 0.21 mmol, 0.8 eq), potassium phosphate (110 mg, 0.5182 mmol), and dioxane (2.0 mL) were combined in a sealed vial and heated to 200° C. for 5 minutes. The reaction mixture was cooled to room temperature and partitioned between dichloromethane and saturated aqueous NaCl. The organics were collected, evaporated to dryness, and purified by silica gel chromatography (linear gradient 0-40% ethyl acetate/heptane) to provide N-(1-(5-bromopyrimidin-2-yl)-1H-pyrazol-3-yl)-1-(2-fluorophenyl)cyclopropane-1-carboxamide (73.5 mg, 69%). 1H NMR (300 MHz, DMSO-d6) δ 9.79 (s, 1H), 8.96 (s, 2H), 8.50 (d, J=2.8 Hz, 1H), 7.49-7.36 (m, 2H), 7.23-7.17 (m, 2H), 6.86 (d, J=2.8 Hz, 1H), 1.71-1.55 (m, 2H), 1.27-1.07 (m, 2H) ppm. ESI-MS m/z calc. 401.02875, found 403.15 (M+1).

### N-(1-(3,5-difluoropyridin-2-yl)-1H-pyrazol-3-yl)-1-(2-fluorophenyl)cyclopropane-1-carboxamide (Compound 391)

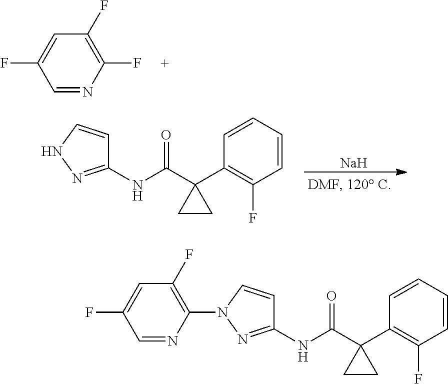

To a slurry of sodium hydride (60% w/w dispersion in oil, 6.5 mg, 0.163 mmol, 1.0 eq) in DMF (2.0 mL) was added N-(1H-pyrazol-3-yl)acetamide (40 mg, 0.163 mmol, 1.0 eq). After gas evolution subsided, 2,3,5-trifluoropyridine (26 mg, 0.196 mmol, 1.2 eq) was added, and the resultant reaction mixture was stirred for 16 h at 120° C. The mixture was cooled to room temperature and partitioned between dichloromethane and water. The organics were collected and evaporated. The crude residue was purified by C18 preparatory HPLC (acetonitrile/water with TFA modifier). The product thus obtained was dissolved in dichloromethane and washed with saturated aqueous sodium bicarbonate solution. The organics were collected, dried (Na2SO4), filtered, and concentrated to provide N-(1-(3,5-difluoropyridin-2-yl)-1H-pyrazol-3-yl)-1-(2-fluorophenyl)cyclopropane-1-carboxamide (16.4 mg, 26% yield). 1H NMR (400 MHz, DMSO-d6) δ 9.53 (s, 1H), 8.27-8.16 (m, 3H), 7.53-7.36 (m, 2H), 7.30-7.16 (m, 2H), 6.85 (d, J=2.7 Hz, 1H), 1.61 (m, 2H), 1.17 (m, 2H) ppm. ESI-MS m/z calc. 358.1041, found 359.06 (M+1).

### N-(1-(5-chloropyridin-2-yl)-1H-pyrazol-3-yl)-1-(2-fluorophenyl)cyclopropane-1-carboxamide (Compound 398)

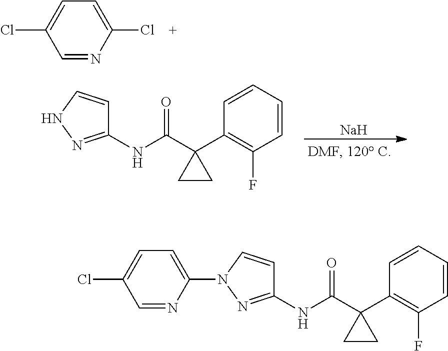

Prepared by the procedure described above for N-(1-(3,5-difluoropyridin-2-yl)-1H-pyrazol-3-yl)-1-(2-fluorophenyl)cyclopropane-1-carboxamide (Compound 391), except that 2,5-dichloropyridine was used as the aryl halide starting material. Product was obtained in 50% yield. 1H NMR (400 MHz, DMSO-d6) δ 9.52 (s, 1H), 8.48 (dd, J=12.4, 2.6 Hz, 2H), 8.05 (dd, J=8.8, 2.6 Hz, 1H), 7.72 (d, J=8.8 Hz, 1H), 7.54-7.35 (m, 2H), 7.26-7.16 (m, 2H), 6.80 (d, J=2.7 Hz, 1H), 1.61 (m, 2H), 1.17 (m, 2H) ppm. ESI-MS m/z calc. 356.0840, found 357.14 (M+1).

### 1-(2-fluorophenyl)-N-(1-(5-fluoropyridin-3-yl)-1H-pyrazol-3-yl)cyclopropane-1-carboxamide (Compound 446)

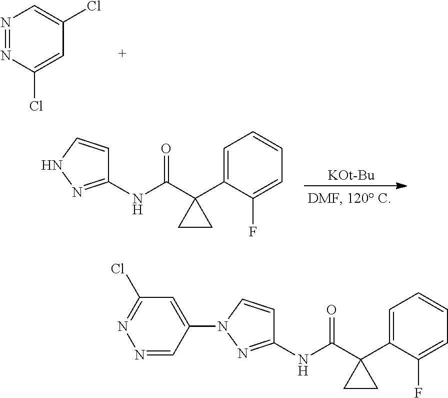 [SMILES: Clc1cnnc(Cl)c1 ; O=C(Nc1cc[nH]n1)C1(c2ccccc2F)CC1 ; O=C(Nc1ccn(-c2cnnc(Cl)c2)n1)C1(c2ccccc2F)CC1 ; "KOt-Bu"]

1-(2-fluorophenyl)-N-(1H-pyrazol-3-yl)cyclopropanecarboxamide (1.046 g, 3.903 mmol), 3,5-dichloropyridazine (620 mg, 3.907 mmol), potassium t-butoxide (450 mg, 4.010 mmol) and DMF (10.0 mL) were combined. The resultant mixture was heated to 100° C. for 16 h. The reaction mixture was partitioned between water and ethyl acetate. The layers were separated, and the aqueous further extracted with ethyl acetate. The combined organic fractions were washed with water and brine, dried (sodium sulfate), filtered, and concentrated. The crude residue was purified by silica gel chromatography (linear gradient of MeOH/methylene chloride containing 0.1% TEA) to provide the desired product (320 mg), though it still contained impurities. A 30 mg of the impure product was purified by C18 preparatory HPLC (acetonitrile/water with TFA modifier) to provide N-[1-(6-chloropyridazin-4-yl)pyrazol-3-yl]-1-(2-fluorophenyl)cyclopropanecarboxamide (trifluoroacetate salt, 13.5 mg). 1H NMR (400 MHz, Methanol-d4) δ 9.58 (d, J=2.3 Hz, 1H), 8.44 (d, J=2.9 Hz, 1H), 8.05 (d, J=2.3 Hz, 1H), 7.54-7.37 (m, 2H), 7.24 (td, J=7.6, 1.2 Hz, 1H), 7.17 (ddd, J=10.4, 8.3, 1.2 Hz, 1H), 7.00 (d, J=2.9 Hz, 1H), 4.84 (s, 1H), 1.70 (q, J=4.2 Hz, 2H), 1.24 (q, J=4.2 Hz, 2H) ppm. ESI-MS m/z calc. 357.08, found 358.13 (M+1).

### 1-(2-fluorophenyl)-N-[1-(6-methoxypyridazin-4-yl)pyrazol-3-yl]cyclopropanecarboxamide (Compound 447)

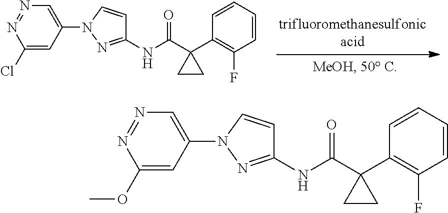

To a solution of Compound 446 (18 mg, 0.049 mmol) in MeOH (1.0 mL) was added trifluoromethanesulfonic acid (10 μL, 0.113 mmol). The resultant solution was heated to 50° C. for 16 h. The solution was directly purified by C18 preparatory HPLC (acetonitrile/water with TFA modifier to provide 1-(2-fluorophenyl)-N-[1-(6-methoxypyridazin-4-yl)pyrazol-3-yl]cyclopropanecarboxamide (trifluoroacetate salt, 3.0 mg, 12% yield) 1H NMR (400 MHz, Methanol-d4) δ 9.33 (d, J=2.2 Hz, 1H), 8.42 (d, J=2.9 Hz, 1H), 7.55-7.40 (m, 3H), 7.30-7.13 (m, 2H), 6.97 (dd, J=2.8, 1.1 Hz, 1H), 4.12 (s, 3H), 1.74-1.66 (m, 2H), 1.24 (q, J=4.2 Hz, 2H) ppm. ESI-MS m/z calc. 353.13, found 354.17 (M+1).

### 2-(hydroxymethyl)-1-phenyl-N-(1-phenyl-1H-pyrazol-3-yl)cyclopropane-1-carboxamide (Compound 444)

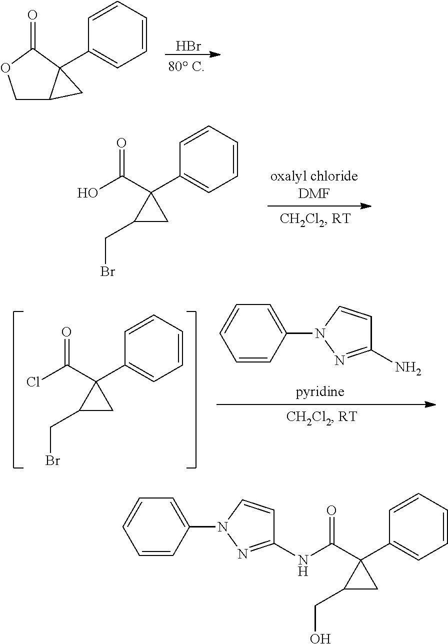

### Step 1

To a stirred solution of HBr (25 mL of 33% w/v, 102.0 mmol) was added 1-phenyl-3-oxabicyclo[3.1.0]hexan-2-one (5 g, 28.70 mmol) portion-wise. Once addition was completed the solution was stirred at 80° C. for 2 hours. The reaction mixture was cooled to room temperature and stirred with 100 g of ice. A solid crashed out and was collected by filtration to yield 2-(bromomethyl)-1-phenyl-cyclopropanecarboxylic acid (7.16 g, 98%) 1H NMR (400 MHz, Chloroform-d) δ 7.47-7.41 (m, 2H), 7.42-7.31 (m, 3H), 3.90 (m, 1H), 3.78 (m, 1H), 2.19 (m, 1H), 1.87 (m, 1H), 1.68 (m, 1H) ppm. ESI-MS m/z calc. 253.99, found 255.01 (M+1).

### Steps 2 & 3

2-(bromomethyl)-1-phenyl-cyclopropanecarboxylic acid (1.6 g, 6.272 mmol) was added to thionyl chloride (1.9 mL, 26.05 mmol) to form a suspension. N,N-dimethylformamide (5 μL, 0.0646 mmol) was added, resulting in gas evolution. The resultant mixture was stirred overnight and concentrated under a stream of nitrogen to remove excess thionyl chloride. The resulting yellow amorphous solid was dissolved in methylene chloride (10.0 mL) and pyridine (1.4 mL, 17.31 mmol). 1-phenylpyrazol-3-amine (1 g, 6.282 mmol) was added portion-wise over 15 minutes, resulting in bubbling/exotherm and formation of a dark-red purple color. The mixture was stirred overnight. LCMS indicated 2-(bromomethyl)-1-phenyl-N-(1-phenylpyrazol-3-yl)cyclopropanecarboxamide as the major component along with some 2-(hydroxymethyl)-1-phenyl-N-(1-phenylpyrazol-3-yl)cyclopropanecarboxamide. Additional dichloromethane (20 mL) was added, and the organics were extracted from water on a phase separation cartridge. The organic phase was evaporated, and the crude residue purified by silica gel chromatography (linear gradient of EtOAc/heptane) to furnish 2-(bromomethyl)-1-phenyl-N-(1-phenylpyrazol-3-yl)cyclopropanecarboxamide (728 mg, 29%) and 2-(hydroxymethyl)-1-phenyl-N-(1-phenylpyrazol-3-yl)cyclopropanecarboxamide (300 mg, 13%). Characterization data for 2-(hydroxymethyl)-1-phenyl-N-(1-phenylpyrazol-3-yl)cyclopropanecarboxamide: 1H NMR (300 MHz, Chloroform-d) δ 7.84 (d, J=2.6 Hz, 1H), 7.75-7.66 (m, 2H), 7.61-7.53 (m, 2H), 7.46-7.32 (m, 4H), 7.32-7.28 (m, 1H), 7.27-7.19 (m, 1H), 6.51 (d, J=2.6 Hz, 1H), 4.65 (dd, J=9.0, 4.5 Hz, 1H), 4.44 (d, J=9.0 Hz, 1H), 2.42 (m, 1H), 1.76 (m, 1H), 1.35 (dd, J=4.9, 4.9 Hz, 1H) ppm.

### Compounds Prepared Via SFC Separation of a Racemate

### 2,2-difluoro-1-phenyl-N-(1-phenyl-1H-pyrazol-3-yl)cyclopropane-1-carboxamide (Compound 143)

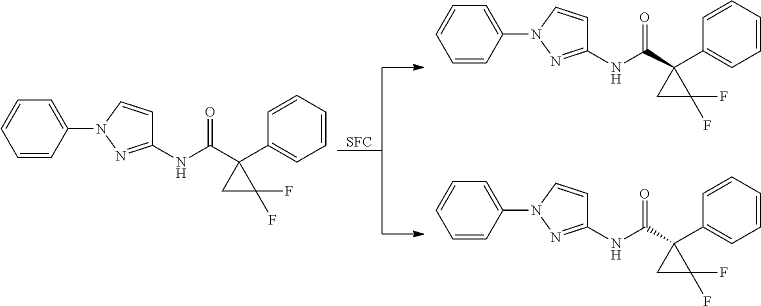

Rel-(R)-2,2-difluoro-1-phenyl-N-(1-phenyl-1H-pyrazol-3-yl)cyclopropane-1-carboxamide and rel-(S)-2,2-difluoro-1-phenyl-N-(1-phenyl-1H-pyrazol-3-yl)cyclopropane-1-carboxamide were prepared by SFC separation of racemic mixture 2,2-difluoro-1-phenyl-N-(1-phenyl-1H-pyrazol-3-yl)cyclopropane-1-carboxamide (Compound 143) using 20×250 mm OJ-H column with isocratic 30% methanol (5 mM ammonia), 70% CO₂ as mobile phase. Absolute configuration of the separated enantiomers was arbitrarily assigned (as indicated with the prefix “rel” in the IUPAC name). The first elution peak was assigned to Compound 366 and the later elution peak was assigned to Compound 367.

### Rel-(R)-2,2-difluoro-1-phenyl-N-(1-phenyl-1H-pyrazol-3-yl)cyclopropane-1-carboxamide (Compound 366)

¹H NMR (400 MHz, DMSO-d₆) δ 11.16 (s, 1H), 8.40 (d, J=2.6 Hz, 1H), 7.76 (m, 2H), 7.65 (m, 2H), 7.43 (m, 5H), 7.28 (m, 1H), 6.74 (d, J=2.6 Hz, 1H), 2.43 (m, 1H), 2.14 (m, 1H) ppm. ESI-MS m/z calc. 339.12, found 340.02 (M+1). [α]_{D} −61.5.

### Rel-(S)-2,2-difluoro-1-phenyl-N-(1-phenyl-1H-pyrazol-3-yl)cyclopropane-1-carboxamide (Compound 367)

¹H NMR (400 MHz, DMSO-d₆) δ 11.16 (s, 1H), 8.39 (d, J=2.6 Hz, 1H), 7.75 (m, 2H), 7.65 (m, 2H), 7.44 (m, 5H), 7.28 (m, 1H), 6.74 (d, J=2.6 Hz, 1H), 2.43 (m, 1H), 2.14 (m, 1H) ppm. ESI-MS m/z calc. 339.12, found 340.02 (M+1). [α]_{D} +62.3.

### 2,2-difluoro-N-(1-(2-fluoropyridin-4-yl)-1H-pyrazol-3-yl)-1-phenylcyclopropane-1-carboxamide (Compound 169)

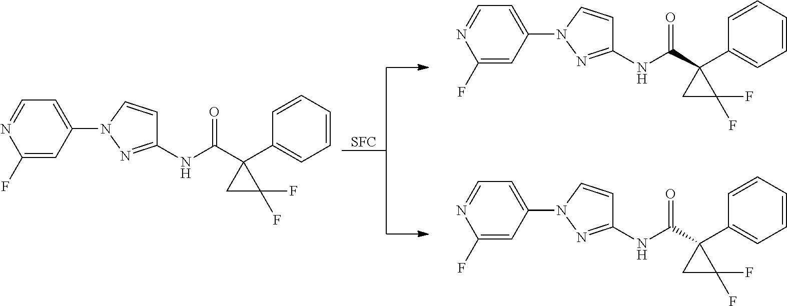

Rel-(R)-2,2-difluoro-N-(1-(2-fluoropyridin-4-yl)-1H-pyrazol-3-yl)-1-phenylcyclopropane-1-carboxamide (Compound 368) and rel-(S)-2,2-difluoro-N-(1-(2-fluoropyridin-4-yl)-1H-pyrazol-3-yl)-1-phenylcyclopropane-1-carboxamide (Compound 369) were prepared by SFC separation of racemic mixture 2,2-difluoro-N-(1-(2-fluoropyridin-4-yl)-1H-pyrazol-3-yl)-1-phenylcyclopropane-1-carboxamide (Compound 169) using 20×250 mm OJ-H column with isocratic 90% hexanes, 10% ethanol/methanol, 0.2% diethylamine as mobile phase. Absolute configuration of the separated enantiomers was arbitrarily assigned (as indicated with the prefix “rel” in the IUPAC name). The first elution peak was assigned to Compound 368 and the later elution peak was assigned to Compound 369.

### Rel-(R)-2,2-difluoro-N-(1-(2-fluoropyridin-4-yl)-1H-pyrazol-3-yl)-1-phenylcyclopropane-1-carboxamide (Compound 368)

¹H NMR (400 MHz, DMSO-d₆) δ 11.35 (s, 1H), 8.65 (d, J=2.8 Hz, 1H), 8.29 (d, J=5.7 Hz, 1H), 7.74 (d, J=5.7 Hz, 1H), 7.64 (d, J=7.0 Hz, 2H), 7.52 (d, J=1.8 Hz, 1H), 7.40 (m, 3H), 6.88 (d, J=2.8 Hz, 1H), 2.44 (m, 1H), 2.19-2.12 (m, 1H) ppm. ESI-MS m/z calc. 358.10, found 359.17 (M+1). [α]_{D} +44.5.

### Rel-(S)-2,2-difluoro-N-(1-(2-fluoropyridin-4-yl)-1H-pyrazol-3-yl)-1-phenylcyclopropane-1-carboxamide (Compound 369)

¹H NMR (400 MHz, DMSO-d₆) δ 11.35 (s, 1H), 8.65 (d, J=2.8 Hz, 1H), 8.29 (d, J=5.7 Hz, 1H), 7.74 (d, J=5.7 Hz, 1H), 7.64 (d, J=7.0 Hz, 2H), 7.52 (d, J=1.8 Hz, 1H), 7.40 (m, 3H), 6.88 (d, J=2.8 Hz, 1H), 2.44 (m, 1H), 2.19-2.12 (m, 1H) ppm. ESI-MS m/z calc. 358.10, found 359.17 (M+1). [α]_{D} −38.4.

### 2,2-dichloro-N-(1-(2-fluoropyridin-4-yl)-1H-pyrazol-3-yl)-1-phenylcyclopropane-1-carboxamide

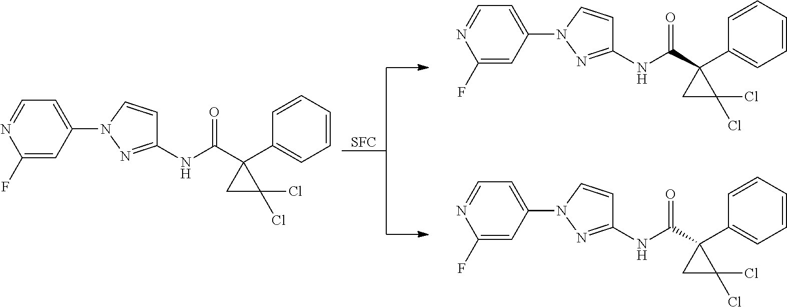

Rel-(R)-2,2-dichloro-N-(1-(2-fluoropyridin-4-yl)-1H-pyrazol-3-yl)-1-phenylcyclopropane-1-carboxamide and rel-(S)-2,2-dichloro-N-(1-(2-fluoropyridin-4-yl)-1H-pyrazol-3-yl)-1-phenylcyclopropane-1-carboxamide were prepared by SFC separation of racemic mixture 2,2-dichloro-N-(1-(2-fluoropyridin-4-yl)-1H-pyrazol-3-yl)-1-phenylcyclopropane-1-carboxamide using 20×250 mm AD-H column with isocratic 30% ethanol (5 mM ammonia), 70% CO₂ as mobile phase. Absolute configuration of the separated enantiomers was arbitrarily assigned (as indicated with the prefix “rel” in the IUPAC name). The first elution peak was assigned to Compound 366 and the later elution peak was assigned to Compound 367.

### Rel-(R)-2,2-dichloro-N-(1-(2-fluoropyridin-4-yl)-1H-pyrazol-3-yl)-1-phenylcyclopropane-1-carboxamide (Compound 370)

¹H NMR (400 MHz, DMSO-d₆) δ 11.39 (s, 1H), 8.65 (d, J=2.8 Hz, 1H), 8.29 (d, J=5.7 Hz, 1H), 7.75-7.66 (m, 3H), 7.52 (m, 1H), 7.40 (m, 3H), 6.88 (d, J=2.8 Hz, 1H), 2.59 (d, J=8.7 Hz, 1H), 2.36 (d, J=8.7 Hz, 1H) ppm. ESI-MS m/z calc. 390.05, found 390.87 (M+1). [α]_{D} −42.1.

### Rel-(S)-2,2-dichloro-N-(1-(2-fluoropyridin-4-yl)-1H-pyrazo-3-yl)-1-phenylcyclopropane-1-carboxamide (Compound 371)

¹H NMR (400 MHz, DMSO-d₆) δ 11.39 (s, 1H), 8.65 (d, J=2.8 Hz, 1H), 8.29 (d, J=5.7 Hz, 1H), 7.75-7.66 (m, 3H), 7.52 (m, 1H), 7.40 (m, 3H), 6.88 (d, J=2.8 Hz, 1H), 2.59 (d, J=8.7 Hz, 1H), 2.36 (d, J=8.7 Hz, 1H) ppm. ESI-MS m/z calc. 390.05, found 390.87 (M+1). [α]_{D} +47.6.

### 2,2-difluoro-1-phenyl-N-(1-(pyridin-4-yl)-1H-pyrazol-3-yl)cyclopropane-1-carboxamide (Compound 419)

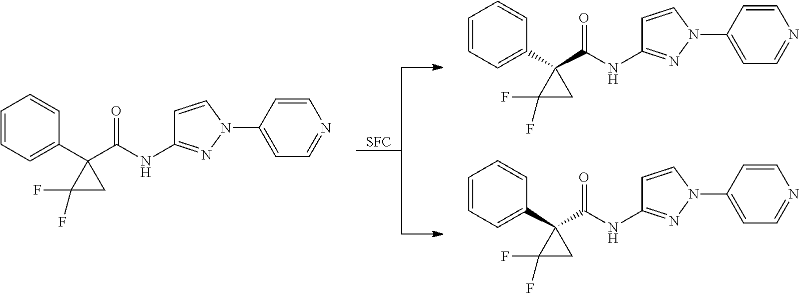

Rel-(S)-2,2-difluoro-1-phenyl-N-(1-(pyridin-4-yl)-1H-pyrazol-3-yl)cyclopropane-1-carboxamide and Rel-(R)-2,2-difluoro-1-phenyl-N-(1-(pyridin-4-yl)-1H-pyrazol-3-yl)cyclopropane-1-carboxamide were prepared by SFC separation of racemic mixture 2,2-difluoro-1-phenyl-N-(1-(pyridin-4-yl)-1H-pyrazol-3-yl)cyclopropane-1-carboxamide (Compound 419) using 20×250 mm OJ-H column with isocratic 60% hexanes/40% isopropanol (0.2% diethylamine) as mobile phase. Absolute configuration of the separated enantiomers was arbitrarily assigned (as indicated with the prefix “rel” in the IUPAC name). The first elution peak was assigned to Compound 433 and the later elution peak was assigned to Compound 434.

### Rel-(S)-2,2-difluoro-1-phenyl-N-(1-(pyridin-4-yl)-1H-pyrazol-3-yl)cyclopropane-1-carboxamide (Compound 433)

ESI-MS m/z calc. 340.11, found 341.06 (M+1).

### Rel-(R)-2,2-difluoro-1-phenyl-N-(1-(pyridin-4-yl)-1H-pyrazol-3-yl)cyclopropane-1-carboxamide (Compound 434)

ESI-MS m/z calc. 340.11, found 341.06 (M+1).

### 2,2-difluoro-N-(1-(5-fluoropyridin-3-yl)-1H-pyrazol-3-yl)-1-phenylcyclopropane-1-carboxamide (Compound 420)

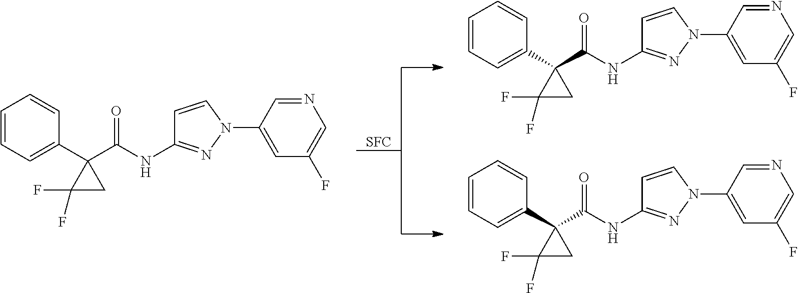

Rel-(S)-2,2-difluoro-N-(1-(5-fluoropyridin-3-yl)-1H-pyrazol-3-yl)-1-phenylcyclopropane-1-carboxamide and Rel-(R)-2,2-difluoro-N-(1-(5-fluoropyridin-3-yl)-1H-pyrazol-3-yl)-1-phenylcyclopropane-1-carboxamide were prepared by SFC separation of racemic mixture 2,2-difluoro-N-(1-(5-fluoropyridin-3-yl)-1H-pyrazol-3-yl)-1-phenylcyclopropane-1-carboxamide (Compound 420) using 10×250 mm AD-H column with isocratic 40% methanol (5 mM ammonia), 60% CO₂ as mobile phase. Absolute configuration of the separated enantiomers was arbitrarily assigned (as indicated with the prefix “rel” in the IUPAC name). The first elution peak was assigned to Compound 435 and the later elution peak was assigned to Compound 436.

### Rel-(S)-2,2-difluoro-N-(1-(5-fluoropyridin-3-yl)-1H-pyrazol-3-yl)-1-phenylcyclopropane-1-carboxamide (Compound 435)

ESI-MS m/z calc. 358.10, found 359.06 (M+1).

### Rel-(R)-2,2-difluoro-N-(1-(5-fluoropyridin-3-yl)-1H-pyrazol-3-yl)-1-phenylcyclopropane-1-carboxamide (Compound 436)

ESI-MS m/z calc. 358.10, found 359.06 (M+1).

### N-(1-(3-chlorophenyl)-1H-pyrazol-3-yl)-2,2-difluoro-1-phenylcyclopropane-1-carboxamide (Compound 421)

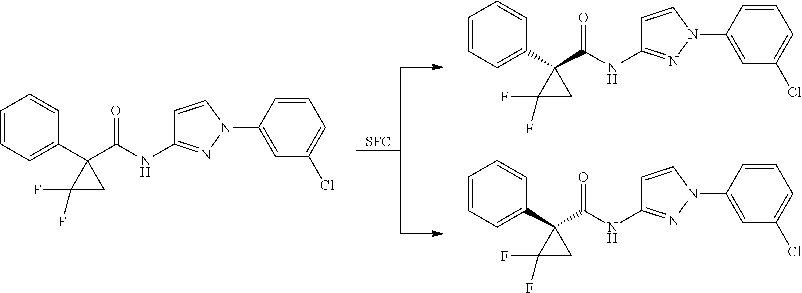

Rel-(S)—N-(1-(3-chlorophenyl)-1H-pyrazol-3-yl)-2,2-difluoro-1-phenylcyclopropane-1-carboxamide and Rel-(R)—N-(1-(3-chlorophenyl)-1H-pyrazol-3-yl)-2,2-difluoro-1-phenylcyclopropane-1-carboxamide were prepared by SFC separation of racemic mixture N-(1-(3-chlorophenyl)-1H-pyrazol-3-yl)-2,2-difluoro-1-phenylcyclopropane-1-carboxamide (Compound 421) using 10×250 mm OJ-H column with isocratic 15% methanol (5 mM ammonia), 85% CO₂ as mobile phase. Absolute configuration of the separated enantiomers was arbitrarily assigned (as indicated with the prefix “rel” in the IUPAC name). The first elution peak was assigned to Compound 437 and the later elution peak was assigned to Compound 438.

### Rel-(S)—N-(1-(3-chlorophenyl)-1H-pyrazol-3-yl)-2,2-difluoro-1-phenylcyclopropane-1-carboxamide (Compound 437)

ESI-MS m/z calc. 373.08, found 374.05 (M+1).

### Rel-(R)—N-(1-(3-chlorophenyl)-1H-pyrazol-3-yl)-2,2-difluoro-1-phenylcyclopropane-1-carboxamide (Compound 438)

ESI-MS m/z calc. 373.08, found 374.05 (M+1).

### 1-(3-fluoropyridin-2-yl)-N-(1-(5-fluoropyridin-3-yl)-1H-pyrazol-3-yl)spiro[2.2]pentane-1-carboxamide (Compound 473)

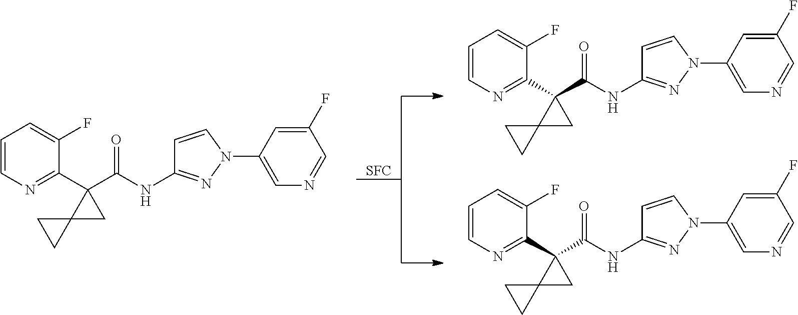

Rel-(S)-1-(3-fluoropyridin-2-yl)-N-(1-(5-fluoropyridin-3-yl)-1H-pyrazol-3-yl)spiro[2.2]pentane-1-carboxamide and Rel-(R)-1-(3-fluoropyridin-2-yl)-N-(1-(5-fluoropyridin-3-yl)-1H-pyrazol-3-yl)spiro[2.2]pentane-1-carboxamide were prepared by SFC separation of racemic mixture 1-(3-fluoropyridin-2-yl)-N-(1-(5-fluoropyridin-3-yl)-1H-pyrazol-3-yl)spiro[2.2]pentane-1-carboxamide (Compound 473) using 10×250 mm IB column with isocratic 40% isopropanol (5 mM ammonia), 60% CO₂ as mobile phase. Absolute configuration of the separated enantiomers was arbitrarily assigned (as indicated with the prefix “rel” in the IUPAC name). The first elution peak was assigned to Compound 499 and the later elution peak was assigned to Compound 500.

### (S)-1-(3-fluoropyridin-2-yl)-N-(1-(5-fluoropyridin-3-yl)-1H-pyrazol-3-yl)spiro[2.2]pentane-1-carboxamide (Compound 499)

1H NMR (400 MHz, Chloroform-d) δ 8.64 (t, J=1.4 Hz, 1H), 8.50 (s, 1H), 8.42 (dt, J=4.6, 1.5 Hz, 1H), 8.28 (d, J=2.5 Hz, 1H), 7.77 (d, J=2.8 Hz, 1H), 7.64 (dt, J=9.5, 2.3 Hz, 1H), 7.39 (ddd, J=9.7, 8.3, 1.4 Hz, 1H), 7.26 (ddd, J=8.4, 4.6, 4.0 Hz, 1H), 6.98 (d, J=2.7 Hz, 1H), 2.22 (d, J=4.9 Hz, 1H), 2.12 (d, J=4.9 Hz, 1H), 1.25-1.16 (m, 2H), 0.97 (dt, J=9.8, 5.2 Hz, 1H), 0.77 (dt, J=8.8, 5.4 Hz, 1H) ppm. ESI-MS m/z calc. 367.12, found 368.06 (M+1).

### (R)-1-(3-fluoropyridin-2-yl)-N-(1-(5-fluoropyridin-3-yl)-1H-pyrazol-3-yl)spiro[2.2]pentane-1-carboxamide (Compound 500)

1H NMR (400 MHz, Chloroform-d) δ 8.64 (t, J=1.5 Hz, 1H), 8.50 (s, 1H), 8.42 (dt, J=4.7, 1.5 Hz, 1H), 8.28 (d, J=2.5 Hz, 1H), 7.77 (dd, J=2.7, 0.5 Hz, 1H), 7.64 (dt, J=9.5, 2.4 Hz, 1H), 7.39 (ddd, J=9.7, 8.3, 1.4 Hz, 1H), 7.26 (ddd, J=8.4, 4.7, 4.0 Hz, 1H), 6.98 (d, J=2.7 Hz, 1H), 2.22 (d, J=4.9 Hz, 1H), 2.12 (d, J=4.7 Hz, 1H), 1.25-1.15 (m, 2H), 0.97 (dt, J=9.8, 5.2 Hz, 1H), 0.77 (dt, J=8.9, 5.3 Hz, 1H) ppm. ESI-MS m/z calc. 367.12, found 368.06 (M+1).

### Compounds Purchased Commercially

The following two compounds were purchased commercially from Enamine: 1-(3-fluorophenyl)-N-(4-methyl-1-phenyl-pyrazol-3-yl)cyclobutanecarboxamide (Compound 372), 1-(o-tolyl)-N-[1-(4-pyridyl)pyrazol-3-yl]cyclopropanecarboxamide (Compound 373), with their structures shown below, respectively.

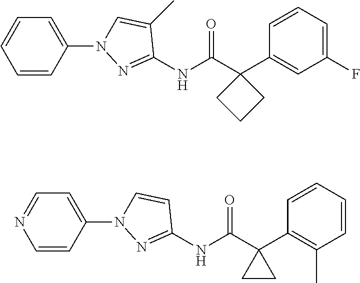 [SMILES: Cc1cn(-c2ccccc2)nc1NC(=O)C1(c2cccc(F)c2)CCC1 ; Cc1ccccc1C1(C(=O)Nc2ccn(-c3ccncc3)n2)CC1]

TABLE E
Compounds prepared via miscellaneous methods described herein.

| | | Average |
|---|---|---|
| Compound | | HEK293 |
| Number | Structure** | IC₅₀ (uM)* |
| 362 | 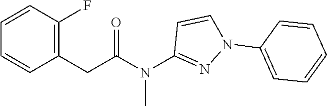 [SMILES: CN(C(=O)Cc1ccccc1F)c1ccn(-c2ccccc2)n1] | >5 |
| 363 | 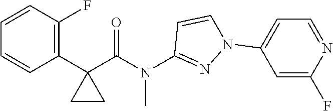 [SMILES: CN(C(=O)C1(c2ccccc2F)CC1)c1ccn(-c2ccnc(F)c2)n1] | >5 |
| 364 | 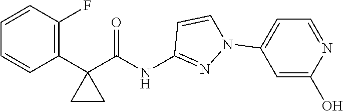 [SMILES: O=C(Nc1ccn(-c2ccnc(O)c2)n1)C1(c2ccccc2F)CC1] | RND |
| 365 | 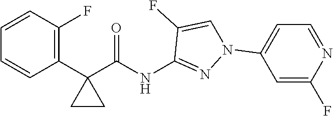 [SMILES: O=C(Nc1nn(-c2ccnc(F)c2)cc1F)C1(c2ccccc2F)CC1] | 0.41 |
| 366 | 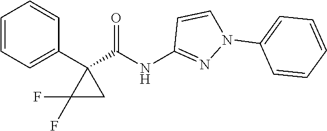 [SMILES: O=C(Nc1ccn(-c2ccccc2)n1)[C@]1(c2ccccc2)CC1(F)F] | 0.21 |
| 367 | 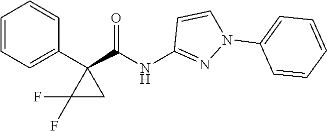 [SMILES: O=C(Nc1ccn(-c2ccccc2)n1)[C@@]1(c2ccccc2)CC1(F)F] | 0.11 |
| 368 | 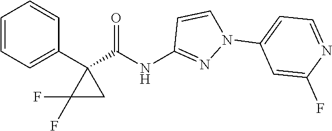 [SMILES: O=C(Nc1ccn(-c2ccnc(F)c2)n1)[C@]1(c2ccccc2)CC1(F)F] | 0.032 |
| 369 | 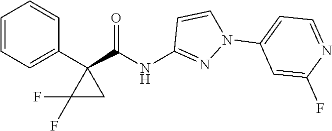 [SMILES: O=C(Nc1ccn(-c2ccnc(F)c2)n1)[C@@]1(c2ccccc2)CC1(F)F] | 0.036 |
| 370 | 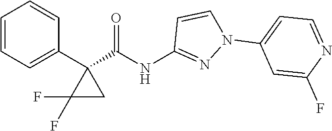 [SMILES: O=C(Nc1ccn(-c2ccnc(F)c2)n1)[C@]1(c2ccccc2)CC1(F)F] | 0.17 |
| 371 | 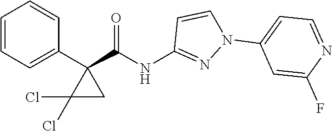 [SMILES: O=C(Nc1ccn(-c2ccnc(F)c2)n1)[C@@]1(c2ccccc2)CC1(Cl)Cl] | 0.72 |
| 372 | 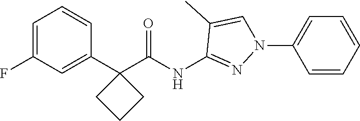 [SMILES: Cc1cn(-c2ccccc2)nc1NC(=O)C1(c2cccc(F)c2)CCC1] | 3.7 |
| 373 | 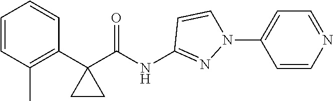 [SMILES: Cc1ccccc1C1(C(=O)Nc2ccn(-c3ccncc3)n2)CC1] | 0.14 |
| 380 | 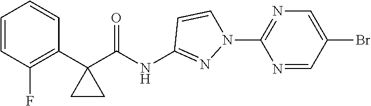 [SMILES: O=C(Nc1ccn(-c2ncc(Br)cn2)n1)C1(c2ccccc2F)CC1] | RND |
| 391 | 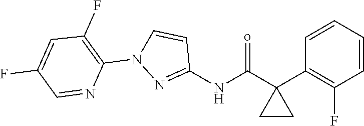 | 0.19 |
| 398 | 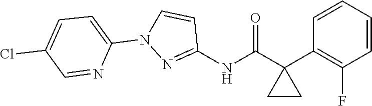 [SMILES: O=C(Nc1ccn(-c2ccc(Cl)cn2)n1)C1(c2ccccc2F)CC1] | >5 |
| 446 | 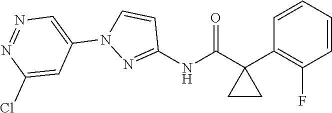 [SMILES: O=C(Nc1ccn(-c2cnnc(Cl)c2)n1)C1(c2ccccc2F)CC1] | RND |
| 445 | 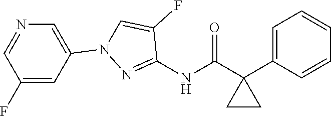 [SMILES: O=C(Nc1nn(-c2cncc(F)c2)cc1F)C1(c2ccccc2)CC1] | 1.4 |
| 447 | 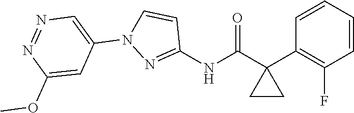 [SMILES: COc1cc(-n2ccc(NC(=O)C3(c4ccccc4F)CC3)n2)cnn1] | >5 |
| 433 | 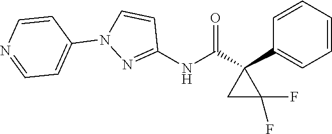 [SMILES: O=C(Nc1ccn(-c2ccncc2)n1)[C@@]1(c2ccccc2)CC1(F)F] | 0.32 |
| 434 | 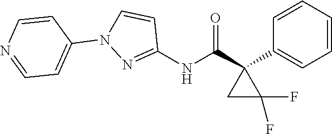 [SMILES: O=C(Nc1ccn(-c2ccncc2)n1)[C@]1(c2ccccc2)CC1(F)F] | 0.34 |
| 435 | 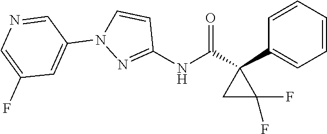 [SMILES: O=C(Nc1ccn(-c2cncc(F)c2)n1)[C@@]1(c2ccccc2)CC1(F)F] | 0.060 |
| 436 | 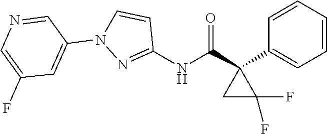 [SMILES: O=C(Nc1ccn(-c2cncc(F)c2)n1)[C@]1(c2ccccc2)CC1(F)F] | 0.18 |
| 437 | 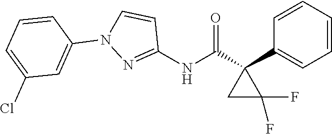 [SMILES: O=C(Nc1ccn(-c2cccc(Cl)c2)n1)[C@@]1(c2ccccc2)CC1(F)F] | 2.4 |
| 438 | 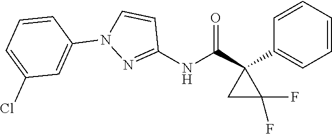 [SMILES: O=C(Nc1ccn(-c2cccc(Cl)c2)n1)[C@]1(c2ccccc2)CC1(F)F] | 0.54 |
| 499 | 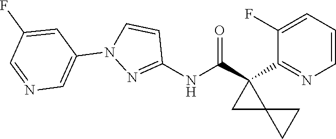 [SMILES: O=C(Nc1ccn(-c2cncc(F)c2)n1)[C@]1(c2ncccc2F)CC12CC2] | 0.74 |
| 500 | 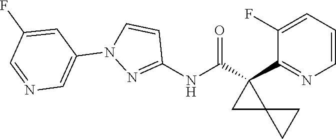 [SMILES: O=C(Nc1ccn(-c2cncc(F)c2)n1)[C@@]1(c2ncccc2F)CC12CC2] | 0.43 |
| 444 | 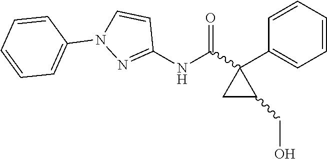 [SMILES: O=C(Nc1ccn(-c2ccccc2)n1)C1(c2ccccc2)CC1CO] | 0.26 |

*The Average HEK293 IC50 reflects the average of one or more replicates.
RND = not determined yet.
**The absolute stereochemistry of chiral compounds was assigned arbitrarily, as explained in the examples
above.

### Example 2. IC₅₀ Assays; In Vitro and In Vivo Efficacy Studies

### Example 2.1. HEK293 VLCFA-LPC IC₅₀ Determination

HEK293 cells were treated with compounds, such as those listed in Tables A-E of Example 1, using the representative manual protocols described below. The protocols below were also adapted to a semi-automated protocol using standard methods in the art.

Cell Culture Growth Conditions:

HEK293 cells were maintained in FreeStyle F17 media (Gibco # A13835) supplemented with PenStrep (1%, Gibco #15070-063), Glutamax (2%, Gibco #35050-061), and Pluronic (0.1%, Gibco #24040-032) (“supplemented media”). Suspension cultures were grown in disposable Erlenmeyer flasks at about 120 rpm, 37° C., 5% CO₂, and 80% humidity. Cell densities were kept between about 0.5 and 3 million cells per mL, in about 50-200 mL per flask.

Treatment of Cells with Compounds Provided Herein:

The cells were treated with compounds using either a total of 900 uL cell media volume (high-volume assay) or a total of 200 uL cell media volume (low-volume assay). In the high volume assay, 450 μL of supplemented media plus 13C-acetate (1.0 mg/mL, Sigma Aldrich #282014) were added to 0.5 μL of a compound (such as those in Tables A-E) in DMSO in a polypropylene v-bottom plate (Costar #3363) in 1 of 3 dilution schemes. The contents of each well were mixed and transferred to a sterile polypropylene deep-well v-bottom plate (Costar #3960). 450 μL of cultured HEK293 cells in supplemented media at a density of 1.0 million cells/mL was added to each well. In the low volume assay, 100 μL of supplemented media plus 13C-acetate (1.0 mg/mL, Sigma Aldrich #282014) were added to either 0.1 μL or 1.0 uL of a compound (such as those in Tables A-E) in DMSO in a polypropylene v-bottom plate (Costar #3363) in 1 of 3 dilution schemes. 100 μL of cultured HEK293 cells in supplemented media at a density of 1.0 million cells/mL was added to each well. The high volume and low volume plates were sealed with either AirPore Tape Sheets (Qiagen #19571) or Duetz plate covers to control evaporation and placed into a shaking incubator at 225 rpm, 37° C., 5% CO2, and 80% humidity for 48 hours. For both the high and low volume assays the 3 dilution schemes used were as follows:

a) Top dose of 5 uM with a 2.5 fold dilution scheme across 9 points to generate a 10-point IC50 curve
b) Top dose of 5 uM with a 2.5 fold dilution scheme across 7 points to generate a 8-point IC50 curve
c) Top dose of 0.2 uM with a 2.5 fold dilution scheme across 7 points to generate a 8-point IC50 curve

Following incubation, treated cells were harvested by centrifugation at 1690×g for 10 minutes. In the high volume assay 200 uL treated cells were transferred to a polypropylene v-bottom plate (Costar #3363) prior to centrifugation. In the low volume assay the incubation plate was centrifuged directly, without a transfer step. The supernatant was then discarded and the analytes were extracted using 1 of 2 different extraction schemes. In the first scheme, the cell pellet was visibly broken up by mixing the cell pellet up and down in 100 μL of hexane/isopropanol (60:40) 20 times. The resulting mixture was transferred to a 0.45 μm Durapore membrane (Millipore #MSH VN4510) atop a polypropylene v-bottom plate (Costar #3363) and filtered by centrifugation at 1690×g for 5 minutes. 120 μL of n-butanol containing 10 nM C13:0 lysophosphatidylcholine was added to the filtrate as an injection control standard, then the entire volume was transferred to a new Durapore membrane/v-bottom plate. In the second scheme, the cell pellet was visibly broken up by mixing the cell pellet up and down in 180 μL of methanol containing 10 nM C13:0 lysophosphatidylcholine 20 times. The resulting mixture was transferred to a 0.45 μm Durapore membrane (Millipore #MSH VN4510) atop a polypropylene v-bottom plate and filtered by centrifugation at 1690×g for 5 minutes. In both schemes, the plates were then sealed with pierceable capmats (Micronic MP53017) and stored at −20 OC until analyzed.

UHPLC/Mass Spectrometry Readout:

The filtered organic extraction was analyzed with a 1290 Agilent Infinity Series UHPLC coupled to an ABI Sciex QTrap 6500 mass spectrometer. Separation of the derivatized VLCFA, lysophosphatidylcholine, of varying chain lengths (e.g., C16:0, C18:0, C20:0, C22:0, C24:0, and C26:0) was achieved using an Ascentis Express HILIC column (2.7 micron, 5 cm×2.1 mm, Sigma #53934-U). The UHPLC mobile phases consisted of 100% water with 20 mM ammonium formate (solvent A) and acetonitrile (90%)/water (10%) with 20 mM ammonium formate (solvent B). The peak area for the mass spectrometry transition monitoring 13C-labeled C26:0 lysophosphatidylcholine (638.500/104.100 m/z) was used to generate IC₅₀ values by fitting the data to a four parameter dose response (Y=Bottom+(Top-Bottom)/(1+10̂((Log IC₅₀−X)*Hill Slope)). In dilution scheme a), peak areas for the 13C-labeled C26:0 were normalized to the median signal of the lowest tested concentration (negative control). In dilution schemes b) and c), peak areas for the 13C-labeled C26:0 were normalized between the average signal of 8 DMSO-treated wells (negative control) and the average signal of 8 established C26:0 LPC-lowering compound-treated wells (positive control). IC₅₀ values were generated using either GraphPad Prism (La Jolla, Calif.) or GeneData Analyzer Software (Basel, Switzerland). IC₅₀ values for a set of control compounds were found to be within acceptable variance regardless of the assay volume, extraction scheme, or dilution scheme utilized.

### Example 2.2. Reduction in C26:0 LPC Concentration in Human HEK and Patient Cells In Vitro

Lysophosphatidylcholine (LPC) VLCFA were generated from straight chain VLCFA (SC-VLCFA) and were used clinically for newborn screening (Vogel et al., Mol. Genet. Metab. (2015) 114(4):599-603). In vitro efficacy studies were performed by measuring LPC VLCFA level (measured as LPC synthesis) in various cell lines, specifically in 1) human HEK cells, 2) patient derived cells, and 3) human microglia, which are disease relevant CNS cells. Compound 87's dose response relationships and IC₅₀ values were measured in HEK cells, primary patient fibroblasts, immortalized patient lymphocytes, and a human microglial cell line. To measure LPC VLCFA synthesis, the foregoing cells were grown in the presence of ¹³C labeled acetate (13C Labeled sodium acetate; Sigma Aldrich #282014) and Compound 87 (prepared in DMSO) for about 48 hours. Primary patient fibroblasts and immortalized primary patient lymphocytes were acquired from the Coriell Cell Repository at the Coriell Institute for Medical Research.

Hek293 Cells:

HEK293 cell culture protocol and treatment with compound, such as Compound 87, was described in example 2.1.

Human Microglia:

Immortalized human microglia (Applied Biological Materials (ABM); catalog #T0251; Richmond BC, Canada) were grown and sub-cultured following the subculturing protocols from ABM except DMEM (high glucose, pyruvate; LifeTech Cat. No. 11995) was used instead of Prigrow III medium and standard tissue culture grade flasks and plates were used. Microglia cells were grown to about 80% confluence and the media was aspirated and washed once with DPBS. TrypIE (or trypsin) was added and incubated for about 5 min until the cells detached. An equal volume of media was used to neutralize the detachment media and the cells were collected and counted. The cells were spun down at 1000 rpm for 5 min and brought back up in complete media and plated as required at the desired density the day before treatment.

Cell assays for microglia cells were run in 12 well tissue culture treated plates. Assays run in 12 well plates were done either in 900 or 1000 ul of media plus Compound 87, which was added to 12 well plated by changing the media with media containing 1 mg/ml ¹³C-Sodium acetate. Cells were treated with Compound 87 for about 2 days at a dose of 2 uM, along with a 2-fold dilution scheme across 11 points to generate a 12 points IC50 curve. After about 2 days compound treatment, the cells were harvested.

Upon the completion of the compound treatment, the media (with compound treatment) was aspirated from the well. About 1-2 ml of DPBS was added to wash the cells. 100 ul of TrypIE was added to the cells and allowed to incubate at room temperature or 37° C. for 5 min. The cells were scraped and transferred to a polypropylene V-bottomed 96 well plate. Each well was then washed with another 100 ul of DPBS, scraped and transferred again to the same polypropylene V-bottomed 96 well plate. The polypropylene plate was then centrifuged at 3000 rpm for 10 minutes. The supernatant was then removed. The plate was sealed with a plate tape and put at −80° C. for further VLCFA extraction and VLCFA quantitation on LC-MS, as described below.

B-Lymphocytes:

Immortalized primary patient lymphocytes cell lines (cell lines GM13496, GM13497, and GM04674) were obtained from the Coriell Cell Repository at the Coriell Institute for Medical Research. Lymphocytes were cultured and plated at a desired cell density, such as 1×10⁵ cells/well. Media used was RPMI+2 mM Glutamine or Glutamax+15% FBS (not heat inactivated). Assays were completed similar to the protocol described for microglia cells except that round bottom 96 well plates were used and the assays were performed in 200 ul of complete media with 1 mg/ml 13C-sodium acetate. Lymphocytes were treated with Compound 87 for about two days at the following doses: 2, 0.964, 0.464, 0.224, 0.108, 0.0519, 0.025, 0.0121, 0.0058, 0.0028, 0.00135, and 0.00065 μM. At completion of the assay, lymphocytes were harvested by spinning down at 3000 rpm for 10 min and removing the supernatant. The plate was sealed with a plate tape and put at −80° C. for further VLCFA extraction and VLCFA quantitation on LC-MS, as described below.

Patient Fibroblasts:

Primary patient fibroblasts were obtained from Coriell Institute for Medical Research. Fibroblasts were cultured by passing the cells at about 95% confluency (nearly 100%), aspirating the media, washing the plate with DPBS, adding TrypIE (preferred) or trypsin to dislodge the cells and leave at 37° C. for 5-10 min, collecting cells with at least as much volume as TrypIE used to neutralize the trypsin, count the cells and calculating cell density. Fibroblasts were plated at a desired cell density, such as 1.9×10⁵ cell/well, in 12 well plates the day before dosing with Compound 87. 13C-acetate (1.0 mg/mL, Sigma Aldrich #282014) and Compound 87 were diluted in media and simultaneously added to a 50% confluent fibroblast culture in 12 well plates, following removal of the growth media. The cells were incubated at 37° C., 5% CO₂, and 80% humidity for 48 hours with Compound 87 at the following doses: 2, 1, 0.5, 0.25, 0.125, 0.0625, 0.03125, 0.015625, 0.0078125, 0.00390625, 0.001953125, and 0.000976563 μM. Upon the completion of the compound treatment, the cells were harvested similarly to the protocol described for microglia. The plate was sealed with a plate tape and put at −80° C. for further VLCFA extraction and VLCFA quantitation on LC-MS, as described below.

VLCFA Extraction and Quantitation on LCMS:

Treated cells were transferred to a polypropylene v-bottom plate and then centrifuged at 1690×g for 10 minutes. The supernatant was discarded and the cell pellet was disrupted by trituration in 100 uL of hexane (60%)/isopropanol (40%). The resulting mixture was transferred to a 0.45 um Durapore membrane (Millipore #MSH VN4510) atop a polypropylene v-bottom plate and filtered by centrifugation at 1690×g for 5 minutes. 120 uL of n-butanol containing 10 nM C13:0 lysophosphatidylcholine was added to the filtrate, then the entire volume was transferred to a new Durapore membrane/v-bottom plate. The resulting mixture was filtered as before followed by centrifugation at 1690×g for 10 minutes. The plates were then sealed with pierceable capmats (Micronic MP53017) and stored at −20° C. until further analyzed using UPHLC/Mass Spectrometry Readout, as described above in example 2.1, which measured the integration of ¹³C into lysophosphatidylcholine (LPC) indicated fatty acid elongation. Specifically, C16:0, C18:0, C20:0, C22:0, C24:0, and C26:0 LPC levels were measured via mass spectroscopy as described above and IC₅₀ values indicated half maximal reduction in C26:0 LPC levels.

Results:

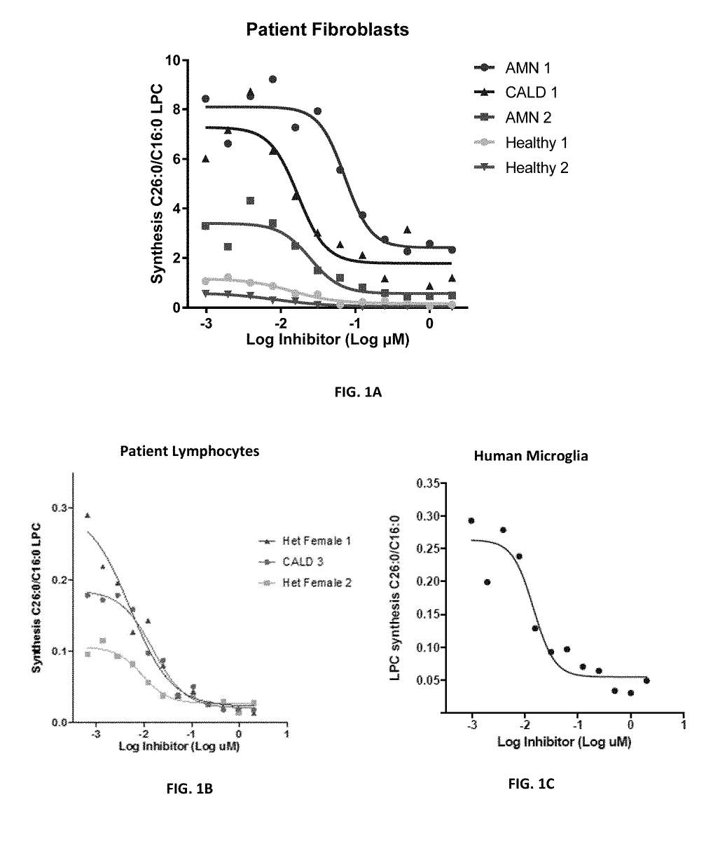

C26:0 LPC levels normalized by C16:0 LPC are shown in FIG. 1A, FIG. 1B, and FIG. 1C. Compound 87 lowered LPC C26:0 levels in human HEK293, patient fibroblasts (CALD1, AMN1, AMN2), patient-derived lymphocytes (CALD, Het Female 1, Het Female 2), and human microglia (see FIG. 1A, FIG. 1B, and FIG. 1C, and Table 5 below). Specifically, Compound 87 reduced C26:0 LPC synthesis in HEK cells, yielding an IC₅₀ of 8 nM. The potency of Compound 87 for ALD patient fibroblasts, lymphocytes, and microglia was similar to the potency for HEK cells.

TABLE 5
Compound 87 Potencies Across Cell Types

| Cell Type | IC₅₀ (nM) |
|---|---|
| HEK293 | 8 |
| Patient Fibroblast (CALD 1) | 17, 23 |
| Patient Fibroblast (AMN 1) | 73, 12 |
| Patient Fibroblast (AMN 2) | 25, 9 |
| Healthy 1 Fibroblast | 14, 32 |
| Healthy 2 Fibroblast | 9, 6 |
| Patient Lymphocyte (CALD) | 15 |
| Patient Lymphocyte (Het Female 1) | 5 |
| Patient Lymphocyte (Het Female 2) | 9 |
| Human Microglia | 11 |

Note:
ALD: adrenoleukodystrophy;
AMN: adrenomyeloneuropathy;
CALD: cerebral adrenoleukodystrophy;
Het: heterozygous;
LPC: lysophosphatidylcholine;
IC₅₀ values indicate half maximal reduction in C26:0 LPC. Each number indicates a separate measurement.

### Example 2.3. Reduction of Plasma C26:0 LPC In Vivo in a Mouse Model, Wild-Type Rats, and Wild-Type Monkeys

Bioanalysis of LPC in Whole Blood and Brain Tissue:

A LC-MS/MS method of analyzing Lysophosphatidylcholine (LPC) in whole blood (dried blood spot card, DBS) and brain tissue samples was developed for measuring the abundance of saturated C16, C18, C20, C22, C24 and C26 LPC in DBS and brain samples. Whole blood was collected with Whatman DMPK-C DBS card at an approximate volume of 20 μL at each time point. Brain tissue was collected at the end point of the study. Samples were prepared and LC-MS/MS analysis was performed as described below.

Sample Preparation for LPC Bioanalysis:

For DBS bioanalysis, the DBS card was punched at 3 mm in diameter using a semi-automated DBS card puncher. To each punched spot 200 μL of pure methanol was added. The vial was vortexed at low speed for 20 minutes and centrifuged at 4000 rpm for 20 minutes. The clear supernatant was injected onto LC-MS/MS for analysis. For brain tissue bioanalysis, brain tissue was collected in a tared homogenization tube pre-filled with metal bead and weighted. To each sample vial two parts weight of methanol was added. The sample was homogenized using Precellys-24 at 5000 rpm for 20 seconds with one cycle. A 100 mg aliquot of homogenate was used for analysis. To each sample vial 400 μL of pure methanol was added. The vial was vortexed at low speed for 20 minutes and centrifuged at 4000 rpm for 20 minutes. The clear supernatant was injected onto LC-MS/MS for analysis.

LC-MS/MS Analysis:

The supernatant obtained from each sample was injected into a LC-MS/MS system (Agilent Technologies, Santa Clara, Calif. and Applied Biosystems, Framingham, Mass.) for analysis. All six LPC components (C16:0, C18:0, C20:0, C22:0, C24:0 and C26:0) were chromatographically separated using a Series 1290 binary pump and a Phenomenex (Torrance, Calif.) Kinetex C18 analytical column (2.1×100 mm, 5 μm particle diameter) with a 10-min gradient. A 5% acetonitrile in water solution was used as the aqueous phase and a 40% acetonitrile/60% methanol solution in 1% 2 Mol ammonium acetate was used as the organic mobile phase for achieving the chromatographic analysis. LPCs were detected by an AB Sciex API-6500 triple quadrupole MS with electrospray ionization in the mode of multiple reaction monitoring. Ions of Q1 were monitored at m/z of 496.6, 524.6, 552.6, 580.6, 608.6 and 636.6 for LPC 16:0, LPC 18:0, LPC 20:0, LPC 22:0, LPC 24:0 and LPC 26:0, respectively. A common Q3 ion m/z of 184.2 was used for all LPC analyses. C16:0 LPC levels were expressed as a concentration. All other LPC levels were expressed relative to C16. A one-way ANOVA with Dunnett's multiple comparisons test was performed to assess differences in LPC levels among the different groups. A value of P≤0.05 was considered statistically significant. All statistical analyses were conducted using Prism Software version 7.01 (GraphPad, La Jolla, Calif.).

Dosing in ABCD1 Knockout Mice:

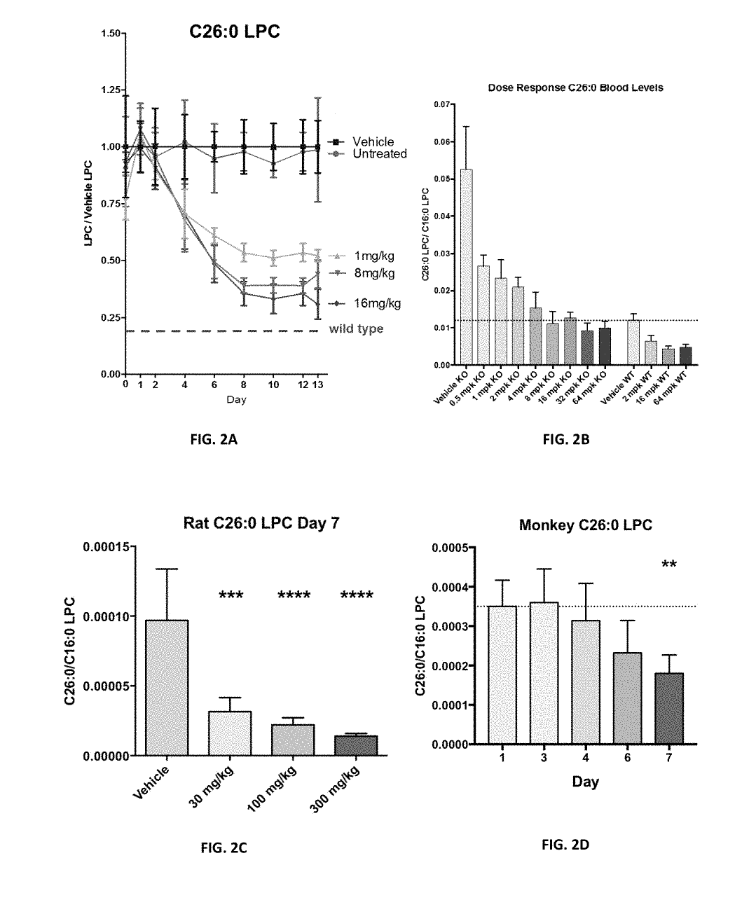

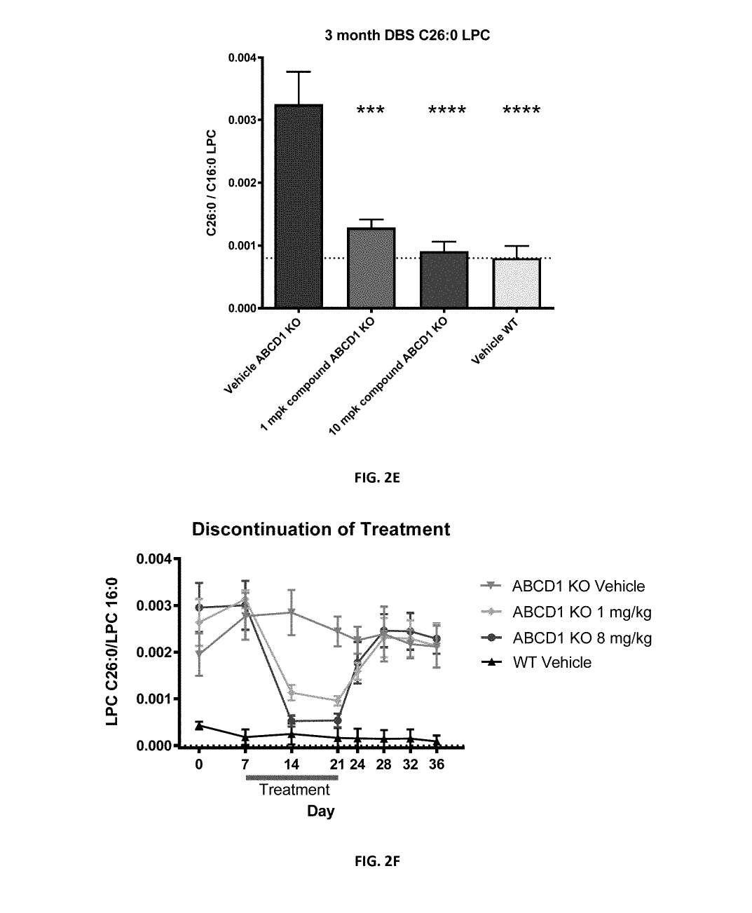

To determine the effect of Compound 87 on blood VLCFA levels, Compound 87 was administered to ABCD1 knockout (KO) mice, a model that reproduces the C26:0 VLCFA accumulation observed in ALD patients. Specifically, Compound 87 was administered orally (PO) QD at 1, 8, or 16 mg/kg to ABCD1 KO mice (n=5 per group). DBS were collected on day 0 (pre-dosing), and daily through 14 days of dosing. DBS cards were stored at 4° C. in sealed ziplock bags with desiccant until they could be analyzed for LPC using the sample preparation and LC-MS/MS as described above. The vehicle used was 2% D-α-Tocopherol polyethylene glycol 1000 succinate (TPGS) and Compound 87 doses were prepared in 2% TPGS. ABCD1 KO mice showed 5-fold higher blood C26:0 LPC levels than WT mice, consistent with the elevations seen in human ALD patients (Van debeek 2016). Interperitoneal dosing, at 2 or 20 mg/kg (data not shown) or oral (PO) dosing at 1, 8, or 16 mg/kg (FIG. 2A) yielded similar results. A dose response was observed between 1 and 8 mg/kg. Plasma C26:0 LPC levels dropped over the first 8 days before plateauing at near WT baseline levels. FIG. 2A shows LPC/vehicle LPC levels (C26:0 LPC levels were normalized to C16:0 LPC levels and vehicle controls) for ABCD1 knockout mice without treatment, vehicle, 1, 8, or 16 mg/kg Compound 87 PO QD daily for 14 days. Error bars indicate standard deviation.

Daily Oral Dosing in ABCD1 Knockout Mice:

To establish the dose response relationship, WT and ABCD1 KO mice were treated with Compound 87 at doses ranging from 0.5 to 64 mg/kg PO once daily (QD) for 28 days (FIG. 2B). The vehicle used is 2% D-α-Tocopherol polyethylene glycol 1000 succinate (TPGS) and Compound 87 doses were prepared in 2% TPGS. Mice were dosed daily (QD) orally (PO) with Compound 87 for 28 days (n=5 mice per group). DBS were collected (n=2 per mouse per time point) and DBS cards were stored at 4° C. until they could be analyzed for lysophosphatidyl cholines (LPCs). DBS samples were prepared and analyzed using LC-MS/MS as described above.

The lowest dose tested, 0.5 mg/kg, yielded a statistically significant reduction in C24:0 and C26:0 LPC levels compared to vehicle controls (50% reduction, one-way ANOVA with Dunnett's multiple comparisons test, p=0.0001). The dose response in ABCD1 KO mice plateaued with a reduction of approximately 75% in C26:0 LPC levels between the 4 mg/kg and 8 mg/kg doses. Blood area under the concentration time-curves (AUCs) were 1951 (±289) ng·h/ml and 3487 (±657) ng·h/ml at the 4 mg/kg and 8 mg/kg doses, respectively. This maximal effect plateau arose at approximately WT baseline LPC levels. WT mice treated with Compound 87 also showed a reduction in VLCFA levels following Compound 87 treatment. The maximal effect plateau in WT mice was reached between the 2 mg/kg and 16 mg/kg doses, and resulted in about a 65% reduction in C26:0 LPC levels to below baseline levels. In FIG. 2B, P value versus ABCD1 KO vehicle controls was 0.0001 at 0.5 mg/kg and higher doses (P≤0.0001); error bars indicated standard deviation.

Reduction of Plasma C26:0 LPC In Vivo in Rats and Monkeys:

Compound 87 was dosed PO (orally by oral gavage) QD at 30, 100, and 300 mg/kg in wild-type (WT) rats (n=5) for 7 days (FIG. 2C). The lowest dose tested in rats, 30 mg/kg, yielded about a 65% reduction in C26:0 LPC levels compared to vehicle controls. The 100 and 300 mg/kg doses yielded about 75% and about 85% reductions, respectively compared to vehicle controls. C26:0 LPC levels in the blood were reduced to below WT baseline. The vehicle used was 5% TPGS and Compound 87 doses were prepared in 5% TPGS. Dried Blood Spot (DSB) samples were collected on day 7, at termination of the experiment. DBS cards were stored at 4° C. until they could be analyzed for LPC. DBS samples were prepared and analyzed using LC-MS/MS as described above.

Compound 87 was dosed PO QD at 30 mg/kg in wild-type male cynomolgus monkeys (n=5) for 7 days (FIG. 2D) and showed about a 50% reduction in blood C26:0 LPC after 7 days of dosing. The vehicle used was 2% TPGS and Compound 87 doses were prepared in 2% TPGS. Dried Blood Spot (DSB) samples were collected at 0.25, 0.5, 1, 2, 4, 8 and 24 hours post dose on Day 1 and Day 7, respectively. In addition, DSB samples were collected for all animals prior to dosing on study Days 3, 4 and 6. DBS cards were stored at 4° C. until they could be analyzed for VLCFAs. DBS samples were prepared and analyzed using LC-MS/MS as described above.

In FIG. 2C and FIG. 2D, **P≤0.01, ***P≤0.001, ****P≤0.0001, one-way ANOVA with Dunnett's multiple comparisons test; error bars indicate standard deviation.

Long Term Dosing in ABCD1 Knock-Out Mice:

To examine whether continuous dosing maintained efficacy in blood, WT mice were dosed with vehicle (n=6) and female ABCD1 KO mice (n=6 per group) were dosed for 3 months with vehicle or with Compound 87 at 1 or 10 mg/kg PO QD. The vehicle used was 2% TPGS and Compound 87 doses were prepared in 2% TPGS. DBS were collected on day 0 (pre-dose), day 1, and weekly through 12 weeks of dosing. DBS cards were stored at 4° C. in sealed ziplock bags with desiccant until they could be analyzed for VLCFAs. DBS samples were prepared and analyzed using LC-MS/MS as described above. Blood C26:0 LPC levels, depicted as C26:0 LPC/C16:0 LPC level, were assessed (FIG. 2E). A dose response was observed; the 1 mg/kg dose induced approximately a 65% reduction in C26:0 LPC levels in vivo and the 10 mg/kg dose induced approximately a 70% reduction in C26:0 LPC levels in vivo. C26:0 LPC/C16:0 LPC levels in the blood were maintained at near WT levels following 3 months of dosing. A one-way ANOVA with Dunnett's multiple comparisons test yielded P value of <0.001 and 0.0001, respectively for the 1 and 10 mg/kg groups. Error bars indicated standard deviation.

Reversible Reducing Effect on LPC Level:

The C26:0 LPC reducing effect of Compound 87 was found to be reversible. After treating WT mice with vehicle (n=5) and adult female ABCD1 KO mice (n=5 per group) with vehicle, 1 or 8 mg/kg of Compound 87 PO (orally) QD (once per day) for 14 days (i.e., day 7 through day 21), treatments with Compound 87 and vehicle were discontinued and blood LPC levels were assessed for another 2 weeks. The vehicle used was 2% TPGS and Compound 87 doses were prepared in 2% TPGS. DBS were collected (n=2 per mouse per time point) on day 0, day 7 (before dosing with Compound 87 or vehicle), days 14 and 21 (while on Compound 87 treatment or on vehicle), as well as days 24, 28, 32 and 36 (after treatment with Compound 87 or vehicle were discontinued). DBS cards were stored at 4° C. until they could be analyzed for lysophosphatidyl cholines (LPCs). DBS samples were prepared and analyzed using LC-MS/MS as described above. Since this study is longitudinal (multiple time points), a two-way ANOVA was performed to assess differences in LPC levels among the different groups. A value of P≤0.05 was considered statistically significant. All statistical analyses were conducted using Prism Software version 7.01. LPC levels returned to baseline levels in approximately 1 week after compound discontinuation, mirroring the kinetics observed following Compound 87 initiation (FIG. 2F).

### Example 2.4. Reduction of C26:0 LPC and SC-VLCFA Levels in Wild-Type and ABCD1 KO Brains

To examine the effect of Compound 87 on VLCFA levels in the CNS, female ABCD1 KO mice were treated with vehicle, 1 or 10 mg/kg PO QD for 2 weeks (14 days; n=5 per group), 1 month (28 days; n=5 per group), 2 months (56 days; n=6 per group), or 3 months (84 days; n=6 per group). The brain samples used in this study were from the same mice used in the long term dosing study in ABCD1 knock-out mice and WT mice (see Example 2.3). Brain tissue samples were collected after 2, 4, 8, or 12 weeks of dosing with vehicle or Compound 87. Brain samples were frozen at −70° C. and were analyzed for VLCFA (LPC, SC-VLCFA, acyl-carnitines) via liquid chromatography-mass spectrometry (LCMS) as described below. The vehicle used was 2% TPGS and Compound 87 doses were prepared in 2% TPGS.

Levels of VLCFA, including straight chain very long chain fatty acids (SC-VLCFA), acyl carnitines, and lysophosphatidylcholines (LPC), in the brain were examined. SC-VLCFA were expected to be rapidly incorporated into other forms and acyl carnitines were expected to be rapidly degraded, contributing to a short expected half-life for these forms. LPC was expected to integrate into membranes, contributing to a longer expected half-life.

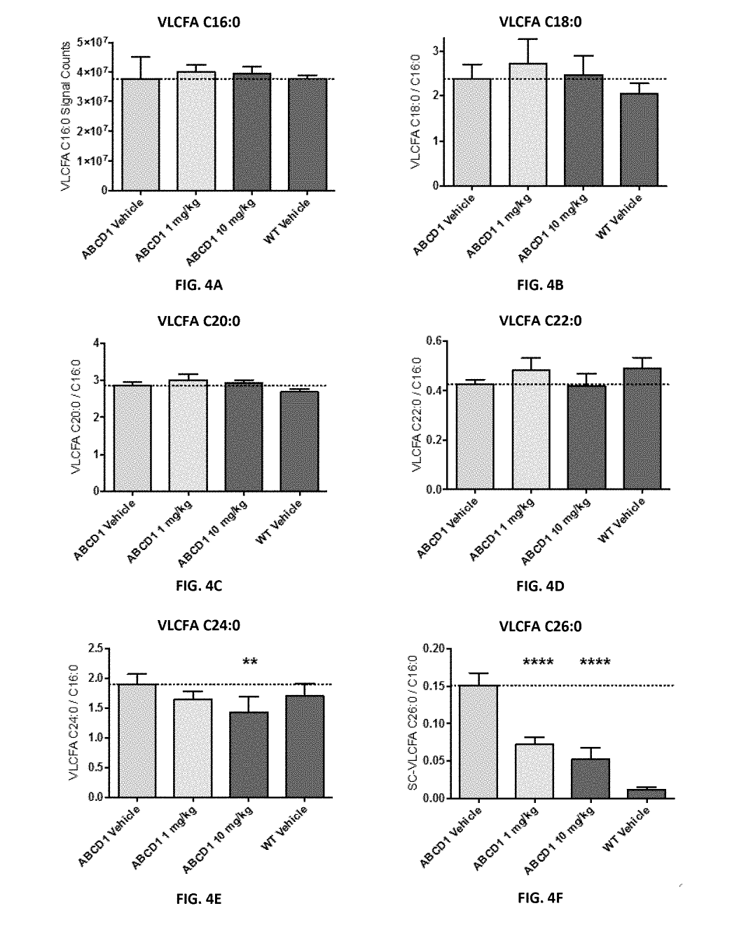

Compound 87 reduced C26:0 SC-VLCFA levels in the brains of ABCD1 KO mice after 2 months of treatment (data not shown), and levels were significantly reduced after 3 months (FIG. 4F). In this experiment, C26:0 SC-VLCFA levels in ABCD1 KO mice were 10 fold higher than in WT mice (Poulos A., et al., Ann. Neurol. (1994) 36(5):741-6; Asheuer M., et al., Hum. Mol. Genet. (2005) 14(10):1293-303). There were no changes in SC-VLCFA levels at either 1 mg/kg or 10 mg/kg dose after 2 weeks of dosing (not shown). The 1 mg/kg dose of Compound 87 reduced C26:0 SC-VLCFA levels by about 30% at 2 months (not shown) and about 50% at 3 months (FIG. 4F). The 10 mg/kg dose yielded a more rapid reduction followed by an apparent plateau, reducing C26:0 SC-VLCFA by about 55% by month 2 (not shown) and by about 65% by month 3 (FIG. 4F). Ten mg/kg of Compound 87 also induced a significant reduction in brain C24:0 SC-VLCFA level after 3 months of dosing (P≤0.01) (FIG. 4E). In FIG. 4, P values versus ABCD1 KO vehicle controls are as follows: *P≤0.05, ** P≤0.01, *** P≤0.001, **** P≤0.0001; and error bars indicated standard deviation.

Compound 87 reduced C26:0 acyl carnitine levels in the brains of ABCD1 KO mice as well. After 2 months of treatment, C26:0 acyl carnitine levels showed about a 50% reduction at 1 mg/kg and about a 70% reduction at 10 mg/kg. Data for acyl carnitine levels are not shown.

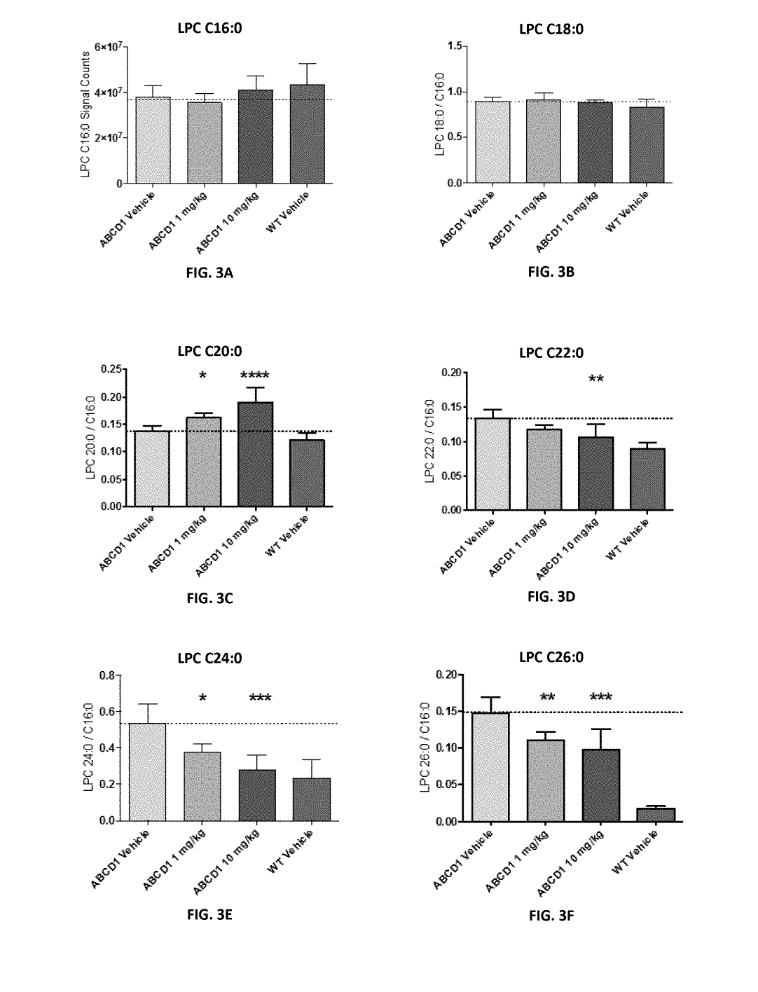

LPC levels in the brains of ABCD1 KO mice showed more modest changes in response to Compound 87. FIG. 3F shows levels of normalized C26:0 LPC in brains of wild-type adult female mice (n=6) treated with vehicle and of adult female ABCD1 KO mice treated with vehicle (n=6), treated with 1 mg/kg of Compound 87 PO QD for 3 months (n=6), and treated with 10 mg/kg Compound 87 PO QD for 3 months (n=6). Brain C26:0 LPC levels in ABCD1 KO mouse were approximately 8 fold higher than in WT mice. There were no changes in LPC levels at either dose after 2 weeks of dosing (not shown). One mg/kg Compound 87 induced about a 30% reduction in brain C26:0 LPC at 2 months (not shown) that was maintained through month 3 (FIG. 3F). Ten mg/kg Compound 87 induced about a 40% reduction in brain C26:0 LPC at 2 months (not shown) and 3 months (FIG. 3F). Both one mg/kg and ten mg/kg of Compound 87 induced a reduction in brain C24:0 LPC levels (normalized by C16:0 LPC levels) (FIG. 3E). P values versus ABCD1 KO vehicle controls are indicated as follows: *P≤0.05, ** P≤0.01, *** P≤0.001, **** P≤0.0001; error bars indicated standard deviation.

These long term brain studies indicated that Compound 87 induced significant reductions in VLCFA levels in the brains of ABCD1 KO mice, a preclinical model of CLD. Specifically, there were significant reductions in brain C26:0 LPC (FIG. 3F) and SC-VLCFA (FIG. 4F) levels at both doses by 3 months of dosing. LPC levels exhibited more modest changes, while acyl carnitines and straight chain VLCFA levels showed robust changes after 8 weeks of dosing.

Brain Sample Preparation:

(i) 3 volumes of MeOH was add to each sample; (ii) homogenized tissue samples with FastPrep (FP120) at 4.5 intensity for 25 seconds; and (iii) aliquoted tissue lysates.

LPC and Acylcarnitine Extraction with CHCl3/MeOH Liquid-Liquid Extraction:

Added 1 mL MeOH, then added 1 mL CH₃Cl to the brain tissue lysates; incubated 30 minutes at room temperature; added 1 mL CHCl₃ and 0.75 mL H₂O; incubated 30 minutes; centrifuged max for 10 minutes; transferred lower layer to new vials; organic phase was dried using Turbo-Vac. The resulting residue was re-constituted with MeOH.

3-Step Chemical Derivatization of SC-VLCFA Using Dimethylaminoethanol (VLCFA-DMAE):

(i) added oxalyl chloride (2 mol/l oxalyl chloride in CH2Cl2, 200 ul) to the dried mixture, incubated at 65° C. for 5 minutes; (ii) added 60 uL dimethylaminoethanol, incubated at 25° C. for 5 minutes and dried down; (iii) added 100 uL methyl iodide, incubated briefly and dried down. The resulting residue was re-constituted with ethanol (EtOH).

LCMSMS Detection of VLCFA (e.g., Spingomyelin (SM) and LPC and Derivatized VLCFA (FA-DMAE)):

LPC Detection:
Column: Discovery C18, 2.1×20 mm
Phase A: 50% MeOH/5 mM AF; Phase B: 2-propanol
MS: 4000 Qtrap operated in ESI MRM positive mode

FA-DMAE Detection:
Column: Synergi Polar RP, 2×150 mm
Phase A: H2O/0.1% FA; Phase B: ACN/0.1% FA
MS: 4000 Qtrap operated in ESI MRM positive mode

### Example 2.5. Thermal Pain Sensitivity in ABCD1 KO Mice in Prophylactic and Therapeutic Dosing Models

ABCD1 KO mice were used as a functional model of AMN. ABCD1 KO mice display a progressive loss of sensitivity to painful thermal stimulus similar to symptoms observed in AMN patients such as decreased sensitivity to touch. To determine the effect of Compound 87 on thermal sensitivity, Compound 87 was dosed PO QD either prophylactically or therapeutically to determine whether ABCD1 KO mice have different latency thresholds for the Plantar test (Hargreaves apparatus) response compared to wild-type (WT) mice.

For the prophylactic study, mice were tested beginning at 10 months of age (before the loss of pain sensitivity) using doses of either 5 or 20 mg/kg. For the therapeutic study, mice were tested beginning at 18 months of age, after there was already a significant loss of pain sensitivity, using doses of either 32 or 64 mg/kg. Mice did not have a significant drop in body weight or any other noticeable adverse effect during Compound 87 treatment in either experiment. The Plantar test (using a Hargreaves apparatus) was used and measured the latency to respond to a thermal stimulus using the following protocol. An individual mouse was placed into an individual compartment with a glass floor for about 10-15 minutes until they were settled. Each individual mouse was given three trials with an infrared source on each hind paw (alternated hind paws each time, and waited 5 minutes between each trial). The infrared source was placed under the glass floor and was positioned by the operator directly beneath the hind paw. A trial was commenced by depressing a key/button which turned on the infrared source and started a digital timer. When a response was observed (paw withdrawal), the key/button was released and the latency to respond was recorded (in seconds).

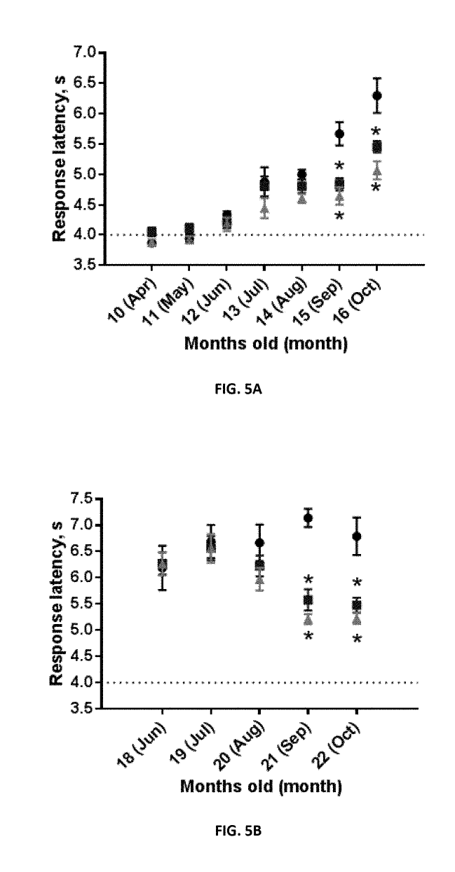

Prophylactic treatment with Compound 87 at 5 or 20 mg/kg reduced the loss of thermal pain sensitivity in ABCD1 KO mice (n=8-10 mice per group) (FIG. 5A). Compound 87 treated mice developed smaller deficits than vehicle treated mice. Dosing was initiated at 10 months of age, before the mice show deficits in thermal sensitivity. Ten-month-old ABCD1 KO mice initially had response latencies around 4 seconds, similar to WT mice (indicated by the dashed horizontal line in FIG. 5A). Mice dosed with vehicle had a significant increase in response latencies over the 6 month period, consistent with a loss of thermal pain sensitivity. Mice dosed with Compound 87 exhibited lower latencies than vehicle treated mice, indicating a restoration or preservation of thermal pain sensitivity and slowing of disease progression. Two-way ANOVA revealed a significant effect of time (p<0.0001), treatment (p<0.0001) and an interaction (p<0.0001).

Therapeutic treatment with Compound 87 reversed the loss of thermal pain sensitivity in older ABCD1 KO mice (n=8-10 mice per group) (FIG. 5B). Dosing was initiated at 18 months of age, after the mice developed deficits in thermal sensitivity, which occurs around 15 months of age. Eighteen-month-old ABCD1 KO mice have response latencies of approximately 6 seconds, which are significantly longer than WT mice (indicated by the dashed horizontal line in FIG. 5B). The Plantar test (using a Hargreaves apparatus) was used and measured the latency to respond to a thermal stimulus using the previously described protocol. Baseline measurements were performed before dosing was initiated and used to randomize mice into treatment groups. Mice dosed with vehicle had a gradual increase in response latencies over several months, consistent with further losses in thermal pain sensitivity as the mice age. Mice dosed with Compound 87 showed a statistically significant improvement in response latencies compared to vehicle treated mice, suggesting slowing or an arrest of disease progression. Therapeutically treated mice showed statistically significant improvements relative to their 18 month baseline scores. Two-way ANOVA revealed a significant effect of time (p<0.0001), treatment (p=0.0053) and an interaction (p<0.0001).

### Example 3. Metabolic Stability of Compound 87

The metabolic intrinsic clearance (CLᵢₙₜ) of Compound 87 was determined in human, monkey, dog, rat, and mouse hepatocytes. Cryopreserved human hepatocytes (Lot Hue50c), monkey hepatocytes (cynomolgus; Lot Cy328), dog hepatocytes (beagle, Lot Db235), rat hepatocytes (Sprague Dawley; NNH), and mouse hepatocytes (CD-1; Lot Mc522) were obtained from ThermoFisher (Paisley, UK). In separate experiments, compound 87 (1 μM) was incubated with hepatocytes from each species (0.5 million cells/mL, suspension) in Dulbecco's Modified Eagle's Medium (DMEM) supplemented with 4-(2-Hydroxyethyl)piperazine-1-ethanesulfonic acid (HEPES, 9 mM) and fructose (2.2 mM) (pH 7.5). Samples were quenched with acetonitrile and analyzed by LC-MS/MS. The mean CLᵢₙₜ for Compound 87 in human, monkey, dog, rat, and mouse hepatocytes after incubation for 4 hours was determined to be ≤2.5, ≤2.5, 7.2, 23.6 and 10.7 μL/min/million cells. Based on these date, Compound 87 was low to moderately metabolized in hepatocytes in mouse, rat, dog, monkey, and human, and the rank order of stability at 1M was approximately human>monkey>dog>mouse>rat. Thus, Compound 87 was shown to have favorable in vitro metabolic stability. The metabolic stability of Compound 87 was not expected.

While a number of embodiments of this invention have been described, it is apparent that the basic examples may be altered to provide other embodiments that utilize the chemical entities, methods, uses, and processes of this invention. Therefore, it will be appreciated that the scope of this invention is to be defined by the appended claims rather than by the specific embodiments that have been represented by way of example herein.

1. A chemical entity, which is a free compound of formula (II.B) or a pharmaceutically acceptable salt thereof, wherein Formula (II.B) has the structure, 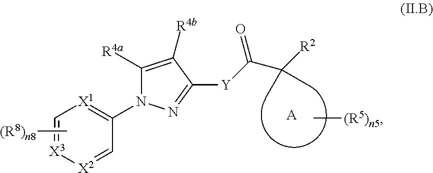
wherein:
A is a cyclopropyl, cyclobutyl, or oxetanyl;
each instance of R⁵ independently is selected from halo, C₁₋₄ alkyl, C₁₋₄ haloalkyl, —(C(R^{J1a}₂))₀₋₂—OH, —(C(R^{J1a}₂))₀₋₂—OR^{J1}, —(C(R^{J1a}₂))₀₋₂—SR^{J1}, —(C(R^{J1a}₂))₀₋₂—NH₂, —(C(R^{J1a}₂))₀₋₂—NHR^{J1}, and —(C(R^{J1a}₂))₀₋₂—NR^{J1}₂
or two geminal R⁵, together with the carbon atom to which they are attached, form a C₃₋₆ cycloalkyl or 3- to 6-membered monocyclic heterocycle containing 1-2 heteroatoms selected from O, N, and S;
n5 is 0, 1 or 2;
R² is phenyl or 5- or 6-membered monocyclic heteroaryl having 1-3 ring heteroatoms independently selected from O, N and S,
wherein each of said phenyl and said 5- or 6-membered monocyclic heteroaryl is unsubstituted or substituted with 1-3 substituents independently selected from halo, C₁₋₄ alkyl, C₁₋₄haloalkyl, —(C(R^{J2a}₂))₀₋₂—OH, —(C(R^{J2a}₂))₀₋₂—OR^{J2}, —(C(R^{J2a}₂))₀₋₂—SR^{J1}, —(C(R^{J2a}₂))₀₋₂—NH₂, —(C(R^{J2}₂)₀₋₂—NHR^{J2}, —(C(R^{J2a}₂))₀₋₂—NR^{J2}₂, —C(O)R^{J2}, and —CN,
wherein each instance of R^{J2} is independently C₁₋₃ alkyl or C₁₋₄ haloalkyl,
wherein each instance of R^{J2a} is independently H, C₁₋₃ alkyl, or C₁₋₄ haloalkyl,
wherein optionally methylenedioxy constitutes a substituent of said phenyl, wherein the methylene unit of the methylenedioxy is unsubstituted or substituted with halo;
one of X¹, X² and X³ is N, and the other two are carbon atoms;
each instance of R⁸ independently is selected from halo, C₁₋₄ alkyl, C₁₋₄ haloalkyl, —(C(R^{J3a}₂))₀₋₂—OH, —(C(R^{J3a}₂))₀₋₂—OR^{J3}, —(C(R^{J3a}₂))₀₋₂—SR^{J3}, —(C(R^{J3a}₂))₀₋₂—NH₂, —(C(R^{J3a}₂))₀₋₂—NHR^{J3}, —(C(R^{J3a}₂))₀₋₂—NR^{J3}₂, —C(O)R^{J3}, and —CN,
wherein each instance of R^{J3} is independently C₁₋₃ alkyl or C₁₋₄ haloalkyl,
wherein each instance of R^{J3a} is independently H, C₁₋₃ alkyl, or C₁₋₄ haloalkyl,
n8 is 0, 1, 2 or 3;
each of R^{4a} and R^{4b} independently is —H, halo, C₁₋₄ alkyl and
Y is —NH— or —N(C₁₋₄ alkyl)-;
wherein 0 to 6 hydrogen atoms of said compound of Formula (II.B) are optionally replaced with deuterium;
provided that the compound of Formula (II.B) is not

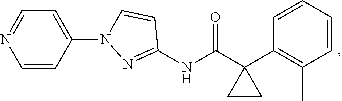 [SMILES: Cc1ccccc1C1(C(=O)Nc2ccn(-c3ccncc3)n2)CC1]

2-11. (canceled)

12. A chemical entity that is a free compound selected from the group consisting of:
2-(2-fluorophenyl)-N-(1-phenylpyrazol-3-yl)acetamide;
1-phenyl-N-(1-phenylpyrazol-3-yl)cyclopropanecarboxamide;
2-(2-fluorophenyl)-N-(4-methyl-1-phenyl-pyrazol-3-yl)acetamide;
N-[1-(2-chloro-4-pyridyl)pyrazol-3-yl]-2-(2-fluorophenyl)acetamide;
N-(1-phenylpyrazol-3-yl)-2-(3-thienyl)acetamide;
2-(4-chlorophenyl)-N-(1-phenylpyrazol-3-yl)acetamide;
2-(2,3-dimethoxyphenyl)-N-(1-phenylpyrazol-3-yl)acetamide;
2-(4-hydroxyphenyl)-N-(1-phenylpyrazol-3-yl)acetamide;
2-(4-hydroxy-3-methoxy-phenyl)-N-(1-phenylpyrazol-3-yl)acetamide;
2-(4-isopropyl phenyl)-N-(1-phenylpyrazol-3-yl)acetamide;
2-[4-fluoro-2-(trifluoromethyl)phenyl]-N-(1-phenylpyrazol-3-yl)acetamide;
1-(o-tolyl)-N-(1-phenylpyrazol-3-yl)cyclopropanecarboxamide;
1-(4-fluorophenyl)-N-(1-phenylpyrazol-3-yl)cyclopropanecarboxamide;
2-(2-fluorophenyl)-N-[1-(4-pyridyl)pyrazol-3-yl]acetamide;
2-(2-fluorophenyl)-N-(1-pyrimidin-5-ylpyrazol-3-yl)acetamide;
2-(4-fluorophenyl)-N-(1-phenylpyrazol-3-yl)acetamide;
N-(1-phenylpyrazol-3-yl)-2-(3,4,5-trifluorophenyl)acetamide;
2-(2,4-difluorophenyl)-N-(1-phenylpyrazol-3-yl)acetamide;
1-(3-chlorophenyl)-N-(1-phenylpyrazol-3-yl)cyclopropanecarboxamide;
(2S)-2-phenyl-N-(1-phenylpyrazol-3-yl)propanamide;
1-(3-fluorophenyl)-N-(1-phenylpyrazol-3-yl)cyclopropanecarboxamide;
1-(2-fluorophenyl)-N-(1-phenylpyrazol-3-yl)cyclopropanecarboxamide;
1-(4-chlorophenyl)-N-(1-phenylpyrazol-3-yl)cyclopropanecarboxamide;
1-(2-chlorophenyl)-N-(1-phenylpyrazol-3-yl)cyclopropanecarboxamide;
1-(4-methoxyphenyl)-N-(1-phenylpyrazol-3-yl)cyclopropanecarboxamide;
1-(3-methoxyphenyl)-N-(1-phenylpyrazol-3-yl)cyclopropanecarboxamide;
(2R)-2-phenyl-N-(1-phenylpyrazol-3-yl)propanamide;
(2R)-2-phenyl-N-(1-phenylpyrazol-3-yl)butanamide;
1-[3-fluoro-5-(trifluoromethyl)phenyl]-N-(1-phenylpyrazol-3-yl)cyclopropanecarboxamide;
2-methyl-2-phenyl-N-(1-phenylpyrazol-3-yl)propanamide;
3-methyl-2-phenyl-N-(1-phenylpyrazol-3-yl)butanamide;
1-(m-tolyl)-N-(1-phenylpyrazol-3-yl)cyclopropanecarboxamide;
1-(4-ethoxyphenyl)-N-(1-phenylpyrazol-3-yl)cyclopropanecarboxamide;
1-phenyl-N-(1-phenylpyrazol-3-yl)cyclopentanecarboxamide;
1-phenyl-N-(1-phenylpyrazol-3-yl)cyclobutanecarboxamide;
1-(3,5-dimethylphenyl)-N-(1-phenylpyrazol-3-yl)cyclopropanecarboxamide;
N-(1-phenylpyrazol-3-yl)-2-(2-thienyl)acetamide;
N-(1-phenylpyrazol-3-yl)-2-(4-pyridyl)acetamide;
N-(1-phenylpyrazol-3-yl)-2-(3-pyridyl)acetamide;
N-(1-phenylpyrazol-3-yl)-2-(2-pyridyl)acetamide;
N-(1-phenylpyrazol-3-yl)-2-(p-tolyl)acetamide;
2-(m-tolyl)-N-(1-phenylpyrazol-3-yl)acetamide;
2-(o-tolyl)-N-(1-phenylpyrazol-3-yl)acetamide;
2-(4-methoxyphenyl)-N-(1-phenylpyrazol-3-yl)acetamide;
2-(3-methoxyphenyl)-N-(1-phenylpyrazol-3-yl)acetamide;
2-(4-ethoxyphenyl)-N-(1-phenylpyrazol-3-yl)acetamide;
2-(2-ethoxyphenyl)-N-(1-phenylpyrazol-3-yl)acetamide;
2-(3-fluorophenyl)-N-(1-phenylpyrazol-3-yl)acetamide;
2-(3-chlorophenyl)-N-(1-phenylpyrazol-3-yl)acetamide;
N-(1-phenylpyrazol-3-yl)-2-[3-(trifluoromethyl)phenyl]acetamide;
N-(1-phenylpyrazol-3-yl)-2-[2-(trifluoromethyl)phenyl]acetamide;
N-(1-phenylpyrazol-3-yl)-2-[4-(trifluoromethoxy)phenyl]acetamide;
N-(1-phenylpyrazol-3-yl)-2-[2-(trifluoromethoxy)phenyl]acetamide;
2-(2,5-dimethylphenyl)-N-(1-phenylpyrazol-3-yl)acetamide;
2-(3-fluoro-4-hydroxy-phenyl)-N-(1-phenylpyrazol-3-yl)acetamide;
2-(3,4-difluorophenyl)-N-(1-phenylpyrazol-3-yl)acetamide;
2-(3,5-difluorophenyl)-N-(1-phenylpyrazol-3-yl)acetamide;
2-(2,5-difluorophenyl)-N-(1-phenylpyrazol-3-yl)acetamide;
2-(2,3-difluorophenyl)-N-(1-phenylpyrazol-3-yl)acetamide;
2-(2,6-difluorophenyl)-N-(1-phenylpyrazol-3-yl)acetamide;
2-(4-methoxy-3-methyl-phenyl)-N-(1-phenylpyrazol-3-yl)acetamide;
2-(3-fluoro-4-methoxy-phenyl)-N-(1-phenylpyrazol-3-yl)acetamide;
2-(3-chloro-4-fluoro-phenyl)-N-(1-phenylpyrazol-3-yl)acetamide;
2-(4-chloro-2-fluoro-phenyl)-N-(1-phenylpyrazol-3-yl)acetamide;
2-(3,4-dimethoxyphenyl)-N-(1-phenylpyrazol-3-yl)acetamide;
2-(2,5-dimethoxyphenyl)-N-(1-phenylpyrazol-3-yl)acetamide;
2-(3,4-dichlorophenyl)-N-(1-phenylpyrazol-3-yl)acetamide;
2-(2,3-dichlorophenyl)-N-(1-phenylpyrazol-3-yl)acetamide;
2-(2,6-dichlorophenyl)-N-(1-phenylpyrazol-3-yl)acetamide;
2-[4-fluoro-3-(trifluoromethyl)phenyl]-N-(1-phenylpyrazol-3-yl)acetamide;
2-[3-fluoro-5-(trifluoromethyl)phenyl]-N-(1-phenylpyrazol-3-yl)acetamide;
2-[3,5-bis(trifluoromethyl)phenyl]-N-(1-phenylpyrazol-3-yl)acetamide;
N-(1-phenylpyrazol-3-yl)-2-(2,3,4-trifluorophenyl)acetamide;
N-(1-phenylpyrazol-3-yl)-2-(2,4,5-trifluorophenyl)acetamide;
N-(1-phenylpyrazol-3-yl)-2-(2,4,6-trifluorophenyl)acetamide;
N-(1-phenylpyrazol-3-yl)-2-(p-tolyl)propanamide;
2-(4-isobutyl phenyl)-N-(1-phenylpyrazol-3-yl)propanamide;
1-(4-chlorophenyl)-N-(1-phenylpyrazol-3-yl)cyclopentanecarboxamide;
2-phenyl-N-(1-phenylpyrazol-3-yl)propanamide;
1-phenyl-N-(1-thiazol-2-ylpyrazol-3-yl)cyclopropanecarboxamide;
N-[1-(3-fluoro-2-pyridyl)pyrazol-3-yl]-1-phenyl-cyclopropanecarboxamide;
N-[1-(2-chloro-4-pyridyl)pyrazol-3-yl]-2-phenyl-acetamide;
N-[1-(2-chloro-4-pyridyl)pyrazol-3-yl]-1-phenyl-cyclopropanecarboxamide;
1-(2-fluorophenyl)-N-(1-pyrimidin-5-ylpyrazol-3-yl)cyclopropanecarboxamide;
2-phenyl-N-[1-(2-pyridyl)pyrazol-3-yl]acetamide;
1-(2-fluorophenyl)-N-[1-(2-fluoro-4-pyridyl)pyrazol-3-yl]cyclopropanecarboxamide;
2-(4-fluorophenyl)-N-[1-(3-fluoro-2-pyridyl)pyrazol-3-yl]acetamide;
2-phenyl-N-(1-thiazol-2-ylpyrazol-3-yl)acetamide;
2-(2-fluorophenyl)-N-(1-thiazol-2-ylpyrazol-3-yl)acetamide;
2-(4-fluorophenyl)-N-(1-thiazol-2-ylpyrazol-3-yl)acetamide;
1-(2-fluorophenyl)-N-(1-thiazol-2-ylpyrazol-3-yl)cyclopropanecarboxamide;
N-[1-(3-fluoro-2-pyridyl)pyrazol-3-yl]-2-phenyl-acetamide;
2-(2-fluorophenyl)-N-[1-(3-fluoro-2-pyridyl)pyrazol-3-yl]acetamide;
1-(2-fluorophenyl)-N-[1-(3-fluoro-2-pyridyl)pyrazol-3-yl]cyclopropanecarboxamide;
2-phenyl-N-(1-pyrimidin-5-ylpyrazol-3-yl)acetamide;
2-(4-fluorophenyl)-N-(1-pyrimidin-5-ylpyrazol-3-yl)acetamide;
1-phenyl-N-(1-pyrimidin-5-ylpyrazol-3-yl)cyclopropanecarboxamide;
2-phenyl-N-[1-(4-pyridyl)pyrazol-3-yl]acetamide;
1-phenyl-N-[1-(4-pyridyl)pyrazol-3-yl]cyclopropanecarboxamide;
1-(2-fluorophenyl)-N-[1-(4-pyridyl)pyrazol-3-yl]cyclopropanecarboxamide;
2-(4-fluorophenyl)-N-[1-(4-pyridyl)pyrazol-3-yl]acetamide;
N-[1-(6-chloro-3-pyridyl)pyrazol-3-yl]-2-phenyl-acetamide;
N-[1-(6-chloro-3-pyridyl)pyrazol-3-yl]-2-(2-fluorophenyl)acetamide;
N-[1-(6-chloro-3-pyridyl)pyrazol-3-yl]-2-(4-fluorophenyl)acetamide;
N-[1-(6-chloro-3-pyridyl)pyrazol-3-yl]-1-phenyl-cyclopropanecarboxamide;
N-[1-(6-chloro-3-pyridyl)pyrazol-3-yl]-1-(2-fluorophenyl)cyclopropanecarboxamide;
1-(4-fluorophenyl)-N-[1-(2-methyl-4-pyridyl)pyrazol-3-yl]cyclopropanecarboxamide;
N-[1-(2-methyl-4-pyridyl)pyrazol-3-yl]-2-phenyl-acetamide;
2-(2-fluorophenyl)-N-[1-(2-methyl-4-pyridyl)pyrazol-3-yl]acetamide;
2-(4-fluorophenyl)-N-[1-(2-methyl-4-pyridyl)pyrazol-3-yl]acetamide;
N-[1-(2-methyl-4-pyridyl)pyrazol-3-yl]-1-phenyl-cyclopropanecarboxamide;
1-(2-fluorophenyl)-N-[1-(2-methyl-4-pyridyl)pyrazol-3-yl]cyclopropanecarboxamide;
N-[1-(2,5-difluoro-4-pyridyl)pyrazol-3-yl]-2-phenyl-acetamide;
N-[1-(2,5-difluoro-4-pyridyl)pyrazol-3-yl]-1-phenyl-cyclopropanecarboxamide;
N-[1-(2,5-difluoro-4-pyridyl)pyrazol-3-yl]-1-(2-fluorophenyl)cyclopropanecarboxamide;
N-[1-(6-methyl-3-pyridyl)pyrazol-3-yl]-2-phenyl-acetamide;
N-[1-(6-methyl-3-pyridyl)pyrazol-3-yl]-1-phenyl-cyclopropanecarboxamide;
2-(2-fluorophenyl)-N-[1-(6-methyl-3-pyridyl)pyrazol-3-yl]acetamide;
1-(2-fluorophenyl)-N-[1-(6-methyl-3-pyridyl)pyrazol-3-yl]cyclopropanecarboxamide;
N-[1-(3-chlorophenyl)pyrazol-3-yl]-2-phenyl-acetamide;
2-(2-fluorophenyl)-N-[1-[2-(trifluoromethyl)-4-pyridyl]pyrazol-3-yl]acetamide;
2-phenyl-N-[1-[2-(trifluoromethyl)-4-pyridyl]pyrazol-3-yl]acetamide;
1-(2-fluorophenyl)-N-[1-(3-fluorophenyl)pyrazol-3-yl]cyclopropanecarboxamide;
N-[1-(3-fluorophenyl)pyrazol-3-yl]-1-phenyl-cyclopropanecarboxamide;
2-(2-fluorophenyl)-N-[1-(3-fluorophenyl)pyrazol-3-yl]acetamide;
N-[1-(3-fluorophenyl)pyrazol-3-yl]-2-phenyl-acetamide;
N-[1-(3-chlorophenyl)pyrazol-3-yl]-1-(2-fluorophenyl)cyclopropanecarboxamide;
N-[1-(3-chlorophenyl)pyrazol-3-yl]-1-phenyl-cyclopropanecarboxamide;
N-[1-(3-chlorophenyl)pyrazol-3-yl]-2-(2-fluorophenyl)acetamide;
N-[1-(4-chlorophenyl)pyrazol-3-yl]-1-(2-fluorophenyl)cyclopropanecarboxamide;
N-[1-(4-chlorophenyl)pyrazol-3-yl]-1-phenyl-cyclopropanecarboxamide;
N-[1-(4-chlorophenyl)pyrazol-3-yl]-2-(2-fluorophenyl)acetamide;
N-[1-(4-chlorophenyl)pyrazol-3-yl]-2-phenyl-acetamide;
1-(2-fluorophenyl)-2,2-dimethyl-N-(1-phenylpyrazol-3-yl)cyclopropanecarboxamide;
2,2-di methyl-1-phenyl-N-(1-phenylpyrazol-3-yl)cyclopropanecarboxamide;
1-(2-fluorophenyl)-N-[1-(3-fluoro-4-pyridyl)pyrazol-3-yl]cyclopropanecarboxamide;
N-[1-(3-fluoro-4-pyridyl)pyrazol-3-yl]-1-phenyl-cyclopropanecarboxamide;
2-(2-fluorophenyl)-N-[1-(3-fluoro-4-pyridyl)pyrazol-3-yl]acetamide;
N-[1-(3-fluoro-4-pyridyl)pyrazol-3-yl]-2-phenyl-acetamide;
2,2-dichloro-1-phenyl-N-(1-phenylpyrazol-3-yl)cyclopropanecarboxamide;
1-phenyl-N-(1-phenylpyrazol-3-yl)cyclohexanecarboxamide;
2,2-difluoro-1-phenyl-N-(1-phenylpyrazol-3-yl)cyclopropanecarboxamide;
3-phenyl-N-(1-phenylpyrazol-3-yl)oxetane-3-carboxamide;
(2R)-2-phenyl-N-(1-phenylpyrazol-3-yl)spiro[2.4]heptane-2-carboxamide;
(2S)-2-phenyl-N-(1-phenylpyrazol-3-yl)spiro[2.3]hexane-2-carboxamide;
2,2-dichloro-N-[1-(2-fluoro-4-pyridyl)pyrazol-3-yl]-1-phenyl-cyclopropanecarboxamide;
N-[1-(2-fluoro-4-pyridyl)pyrazol-3-yl]-1-phenyl-cyclohexanecarboxamide;
N-[1-(2-fluoro-4-pyridyl)pyrazol-3-yl]-3-phenyl-oxetane-3-carboxamide;
(2S)—N-[1-(2-fluoro-4-pyridyl)pyrazol-3-yl]-2-phenyl-spiro[2.4]heptane-2-carboxamide;
(2S)—N-[1-(2-fluoro-4-pyridyl)pyrazol-3-yl]-2-phenyl-spiro[2.3]hexane-2-carboxamide;
2-cyclopentyl-N-[1-(2-fluoro-4-pyridyl)pyrazol-3-yl]-2-phenyl-acetamide;
2-phenyl-N-[1-[4-(trifluoromethyl)phenyl]pyrazol-3-yl]acetamide;
1-(2-fluorophenyl)-N-[1-[4-(trifluoromethyl)phenyl]pyrazol-3-yl]cyclopropanecarboxamide;
2-(2-fluorophenyl)-N-[1-[4-(trifluoromethyl)phenyl]pyrazol-3-yl]acetamide;
1-phenyl-N-[1-[4-(trifluoromethyl)phenyl]pyrazol-3-yl]cyclopropanecarboxamide;
2-phenyl-N-[1-[3-(trifluoromethyl)phenyl]pyrazol-3-yl]acetamide;
2-(2-fluorophenyl)-N-[1-[3-(trifluoromethyl)phenyl]pyrazol-3-yl]acetamide;
1-phenyl-N-[1-[3-(trifluoromethyl)phenyl]pyrazol-3-yl]cyclopropanecarboxamide;
N-[1-(2,5-difluorophenyl)pyrazol-3-yl]-2-phenyl-acetamide;
N-[1-(2,5-difluorophenyl)pyrazol-3-yl]-1-(2-fluorophenyl)cyclopropanecarboxamide;
N-[1-(2,5-difluorophenyl)pyrazol-3-yl]-2-(2-fluorophenyl)acetamide;
N-[1-(2,5-difluorophenyl)pyrazol-3-yl]-1-phenyl-cyclopropanecarboxamide;
(2R)—N-[1-(2-fluoro-4-pyridyl)pyrazol-3-yl]-2-phenyl-spiro[2.4]heptane-2-carboxamide;
N-[1-(2-fluoro-4-pyridyl)pyrazol-3-yl]-2,2-dimethyl-1-phenyl-cyclopropanecarboxamide;
2-ethyl-2-methyl-1-phenyl-N-(1-phenylpyrazol-3-yl)cyclopropanecarboxamide;
2-cyclopentyl-2-phenyl-N-(1-phenylpyrazol-3-yl)acetamide;
2-ethyl-N-[1-(2-fluoro-4-pyridyl)pyrazol-3-yl]-2-methyl-1-phenyl-cyclopropanecarboxamide;
2,2-difluoro-N-[1-(2-fluoro-4-pyridyl)pyrazol-3-yl]-1-phenyl-cyclopropanecarboxamide;
3-methylsulfanyl-2-phenyl-N-(1-phenylpyrazol-3-yl)propanamide;
N-[1-(2-fluoro-4-pyridyl)pyrazol-3-yl]-2-phenyl-butanamide;
3,3-difluoro-N-[1-(2-fluoro-4-pyridyl)pyrazol-3-yl]-1-phenyl-cyclobutanecarboxamide;
N-[1-(2-fluoro-4-pyridyl)pyrazol-3-yl]-2-phenyl-pentanamide;
N-[1-(2-fluoro-4-pyridyl)pyrazol-3-yl]-4-phenyl-tetrahydropyran-4-carboxamide;
2-cyclopropyl-N-[1-(2-fluoro-4-pyridyl)pyrazol-3-yl]-2-phenyl-acetamide 2-phenyl-N-(1-phenylpyrazol-3-yl) pentanamide;
3,3-difluoro-1-phenyl-N-(1-phenylpyrazol-3-yl)cyclobutanecarboxamide;
4-phenyl-N-(1-phenylpyrazol-3-yl)tetrahydropyran-4-carboxamide;
2-cyclopropyl-2-phenyl-N-(1-phenylpyrazol-3-yl)acetamide;
1-(2-fluorophenyl)-N-[1-(5-fluoro-3-pyridyl)pyrazol-3-yl]cyclopropanecarboxamide;
N-[1-(4-fluorophenyl)pyrazol-3-yl]-2-phenyl-acetamide;
1-(2-fluorophenyl)-N-[1-(4-fluorophenyl)pyrazol-3-yl]cyclopropanecarboxamide;
2-(2-fluorophenyl)-N-[1-(4-fluorophenyl)pyrazol-3-yl]acetamide;
N-[1-(4-fluorophenyl)pyrazol-3-yl]-1-phenyl-cyclopropanecarboxamide;
N-[1-(2-fluorophenyl)pyrazol-3-yl]-2-phenyl-acetamide;
1-(2-fluorophenyl)-N-[1-(2-fluorophenyl)pyrazol-3-yl]cyclopropanecarboxamide;
2-(2-fluorophenyl)-N-[1-(2-fluorophenyl)pyrazol-3-yl]acetamide;
N-[1-(2-fluorophenyl)pyrazol-3-yl]-1-phenyl-cyclopropanecarboxamide;
N-[1-(3,4-difluorophenyl)pyrazol-3-yl]-2-phenyl-acetamide;
N-[1-(3,4-difluorophenyl)pyrazol-3-yl]-1-(2-fluorophenyl)cyclopropanecarboxamide;
N-[1-(3,4-difluorophenyl)pyrazol-3-yl]-2-(2-fluorophenyl)acetamide;
N-[1-(3,4-difluorophenyl)pyrazol-3-yl]-1-phenyl-cyclopropanecarboxamide;
N-[1-(4-chloro-3-fluoro-phenylpyrazol-3-yl]-2-phenyl-acetamide;
N-[1-(4-chloro-3-fluoro-phenylpyrazol-3-yl]-1-(2-fluorophenyl)cyclopropanecarboxamide;
N-[1-(4-chloro-3-fluoro-phenyl)pyrazol-3-yl]-2-(2-fluorophenyl)acetamide;
N-[1-(4-chloro-3-fluoro-phenylpyrazol-3-yl]-1-phenyl-cyclopropanecarboxamide;
N-[1-(3-chloro-4-fluoro-phenylpyrazol-3-yl]-2-phenyl-acetamide;
N-[1-(3-chloro-4-fluoro-phenylpyrazol-3-yl]-1-(2-fluorophenyl)cyclopropanecarboxamide;
N-[1-(3-chloro-4-fluoro-phenyl)pyrazol-3-yl]-2-(2-fluorophenyl)acetamide;
N-[1-(3-chloro-4-fluoro-phenylpyrazol-3-yl]-1-phenyl-cyclopropanecarboxamide;
N-[1-(5-fluoro-3-pyridyl)pyrazol-3-yl]-1-phenyl-cyclopropanecarboxamide;
1-(2-fluorophenyl)-N-(1-pyridazin-4-ylpyrazol-3-yl)cyclopropanecarboxamide;
2-(2-fluorophenyl)-N-[1-(2-fluoro-4-pyridyl)pyrazol-3-yl]acetamide;
2-(2-fluorophenyl)-N-[1-(5-fluoro-3-pyridyl)pyrazol-3-yl]acetamide;
N-[1-(5-fluoro-3-pyridyl)pyrazol-3-yl]-2-phenyl-acetamide;
1-(2-fluorophenyl)-N-(1-pyrimidin-4-ylpyrazol-3-yl)cyclopropanecarboxamide;
1-phenyl-N-(1-pyrimidin-4-ylpyrazol-3-yl)cyclopropanecarboxamide;
2-(2-fluorophenyl)-N-(1-pyrimidin-4-ylpyrazol-3-yl)acetamide;
2-phenyl-N-(1-pyrimidin-4-ylpyrazol-3-yl)acetamide;
1-(2-fluorophenyl)-N-[1-(2-pyridyl)pyrazol-3-yl]cyclopropanecarboxamide;
2-(2-fluorophenyl)-N-[1-(2-pyridyl)pyrazol-3-yl]acetamide;
1-phenyl-N-[1-(2-pyridyl)pyrazol-3-yl]cyclopropanecarboxamide;
2-phenyl-N-[1-(3-pyridyl)pyrazol-3-yl]acetamide;
1-(2-fluorophenyl)-N-[1-(3-pyridyl)pyrazol-3-yl]cyclopropanecarboxamide;
1-(2-fluorophenyl)-N-(1-pyrazin-2-ylpyrazol-3-yl)cyclopropanecarboxamide;
2-(2-fluorophenyl)-N-(1-pyrazin-2-ylpyrazol-3-yl)acetamide;
1-(2-fluorophenyl)-N-(1-thiazol-5-ylpyrazol-3-yl)cyclopropanecarboxamide;
2-phenyl-N-(1-pyrazin-2-ylpyrazol-3-yl)acetamide;
N-(1-phenylpyrazol-3-yl)-2-pyrazin-2-yl-acetamide;
N-(1-phenylpyrazol-3-yl)-2-pyrimidin-2-yl-acetamide;
N-[1-(2-fluoro-4-pyridyl)pyrazol-3-yl]-2-pyrimidin-2-yl-acetamide;
N-[1-(2-fluoro-4-pyridyl)pyrazol-3-yl]-1-(3-pyridyl)cyclopropanecarboxamide;
N-[1-(2-fluoro-4-pyridyl)pyrazol-3-yl]-1-(2-pyridyl)cyclopropanecarboxamide;
N-(1-phenylpyrazol-3-yl)-1-(3-pyridyl)cyclopropanecarboxamide;
N-(1-phenylpyrazol-3-yl)-1-(2-pyridyl)cyclopropanecarboxamide;
1-(4-cyanophenyl)-N-[1-(2-fluoro-4-pyridyl)pyrazol-3-yl]cyclopropanecarboxamide;
1-(4-cyanophenyl)-N-(1-phenylpyrazol-3-yl)cyclopropanecarboxamide;
N-[1-(1-methylpyrazol-3-yl)pyrazol-3-yl]-2-phenyl-acetamide;
1-(2-fluorophenyl)-N-[1-(1-methylpyrazol-3-yl)pyrazol-3-yl]cyclopropanecarboxamide;
2-(2-fluorophenyl)-N-[1-(1-methylpyrazol-3-yl)pyrazol-3-yl]acetamide;
N-[1-(1-methylpyrazol-3-yl)pyrazol-3-yl]-1-phenyl-cyclopropanecarboxamide;
N-[1-(2-methoxy-4-pyridyl)pyrazol-3-yl]-2-phenyl-acetamide;
1-phenyl-N-(1-pyrazin-2-ylpyrazol-3-yl)cyclopropanecarboxamide;
1-phenyl-N-[1-(3-pyridyl)pyrazol-3-yl]cyclopropanecarboxamide;
2-(2-fluorophenyl)-N-[1-(3-pyridyl)pyrazol-3-yl]acetamide;
N-[1-(2-fluoro-4-pyridyl)pyrazol-3-yl]-2-pyrimidin-5-yl-acetamide;
N-[1-(2-fluoro-4-pyridyl)pyrazol-3-yl]-1-phenyl-cyclopropanecarboxamide;
N-[1-(2-fluoro-4-pyridyl)pyrazol-3-yl]-1-(4-iodophenyl)cyclopropanecarboxamide;
1-(2-fluorophenyl)-N-(1-pyridazin-3-ylpyrazol-3-yl)cyclopropanecarboxamide;
1-(2-fluorophenyl)-N-(1-pyrimidin-2-ylpyrazol-3-yl)cyclopropanecarboxamide;
1-(2-fluorophenyl)-N-[1-(2-methylpyrimidin-5-yl)pyrazol-3-yl]cyclopropanecarboxamide;
1-(2-cyanophenyl)-N-(1-phenylpyrazol-3-yl)cyclopropanecarboxamide;
1-(3-cyanophenyl)-N-[1-(2-fluoro-4-pyridyl)pyrazol-3-yl]cyclopropanecarboxamide;
1-(3-cyanophenyl)-N-(1-phenylpyrazol-3-yl)cyclopropanecarboxamide;
N-[1-(2-fluoro-4-pyridyl)pyrazol-3-yl]-1-(m-tolyl)cyclopropanecarboxamide;
1-(4-ethoxyphenyl)-N-[1-(2-fluoro-4-pyridyl)pyrazol-3-yl]cyclopropanecarboxamide;
1-(2-cyanophenyl)-N-[1-(2-fluoro-4-pyridyl)pyrazol-3-yl]cyclopropanecarboxamide;
1-(3,5-dimethylphenyl)-N-[1-(2-fluoro-4-pyridyl)pyrazol-3-yl]cyclopropanecarboxamide;
N-[1-(2-fluoro-4-pyridyl)pyrazol-3-yl]-1-(4-methoxyphenyl)cyclopropanecarboxamide;
N-[1-(2-fluoro-4-pyridyl)pyrazol-3-yl]-1-(3-methoxyphenyl)cyclopropanecarboxamide;
1-(2-chlorophenyl)-N-[1-(2-fluoro-4-pyridyl)pyrazol-3-yl]cyclopropanecarboxamide;
N-[1-(2-fluoro-4-pyridyl)pyrazol-3-yl]-1-(o-tolyl)cyclopropanecarboxamide;
1-(2,4-dichlorophenyl)-N-[1-(2-fluoro-4-pyridyl)pyrazol-3-yl]cyclopropanecarboxamide;
1-(3-fluorophenyl)-N-[1-(2-fluoro-4-pyridyl)pyrazol-3-yl]cyclopropanecarboxamide;
1-(4-fluorophenyl)-N-[1-(2-fluoro-4-pyridyl)pyrazol-3-yl]cyclopropanecarboxamide;
1-(3-chlorophenyl)-N-[1-(2-fluoro-4-pyridyl)pyrazol-3-yl]cyclopropanecarboxamide;
1-(4-chlorophenyl)-N-[1-(2-fluoro-4-pyridyl)pyrazol-3-yl]cyclopropanecarboxamide;
N-[1-(4,6-dideuterio-2-methyl-pyrimidin-5-yl) pyrazol-3-yl]-1-(2-fluorophenyl)cyclopropanecarboxamide;
1-(2-fluorophenyl)-N-[1-(2-methoxy-4-pyridyl)pyrazol-3-yl]cyclopropanecarboxamide;
1-(2,5-difluorophenyl)-N-[1-(2-fluoro-4-pyridyl)pyrazol-3-yl]cyclopropanecarboxamide;
1-(2,5-difluorophenyl)-N-(1-phenylpyrazol-3-yl)cyclopropanecarboxamide;
1-(4-chloro-2-fluoro-phenyl)-N-[1-(2-fluoro-4-pyridyl) pyrazol-3-yl]cyclopropanecarboxamide;
1-(4-chloro-2-fluoro-phenyl)-N-(1-phenylpyrazol-3-yl)cyclopropanecarboxamide;
1-(5-chloro-2-fluoro-phenyl)-N-[1-(2-fluoro-4-pyridyl) pyrazol-3-yl]cyclopropanecarboxamide;
1-(5-chloro-2-fluoro-phenyl)-N-(1-phenylpyrazol-3-yl)cyclopropanecarboxamide;
1-(2,6-difluorophenyl)-N-[1-(2-fluoro-4-pyridyl)pyrazol-3-yl]cyclopropanecarboxamide;
1-(2,6-difluorophenyl)-N-(1-phenylpyrazol-3-yl)cyclopropanecarboxamide;
1-(2,3-difluorophenyl)-N-[1-(2-fluoro-4-pyridyl)pyrazol-3-yl]cyclopropanecarboxamide;
1-(2,3-difluorophenyl)-N-(1-phenylpyrazol-3-yl)cyclopropanecarboxamide;
1-(3,5-difluorophenyl)-N-[1-(2-fluoro-4-pyridyl)pyrazol-3-yl]cyclopropanecarboxamide;
1-(3,5-difluorophenyl)-N-(1-phenylpyrazol-3-yl)cyclopropanecarboxamide;
1-(2-chloro-6-fluoro-3-methyl-phenyl)-N-[1-(2-fluoro-4-pyridyl)pyrazol-3-yl]cyclopropanecarboxamide;
1-(2-chloro-6-fluoro-3-methyl-phenyl)-N-(1-phenylpyrazol-3-yl)cyclopropanecarboxamide;
1-(2-chloro-6-fluoro-phenyl)-N-[1-(2-fluoro-4-pyridyl) pyrazol-3-yl]cyclopropanecarboxamide;
1-(2-chloro-6-fluoro-phenyl)-N-(1-phenylpyrazol-3-yl)cyclopropanecarboxamide;
2,2,3,3-tetradeuterio-1-(2-fluorophenyl)-N-[1-(2-fluoro-4-pyridyl)pyrazol-3-yl]cyclopropanecarboxamide;
1-(3-fluoro-2-pyridyl)-N-[1-(2-fluoro-4-pyridyl)pyrazol-3-yl]cyclopropanecarboxamide;
1-(3-fluoro-4-pyridyl)-N-(1-pyrimidin-4-ylpyrazol-3-yl)cyclopropanecarboxamide;
1-(3-fluoro-2-pyridyl)-N-(1-pyrimidin-4-ylpyrazol-3-yl)cyclopropanecarboxamide;
1-(3-fluoro-4-pyridyl)-N-(1-phenylpyrazol-3-yl)cyclopropanecarboxamide;
1-(3-fluoro-2-pyridyl)-N-(1-phenylpyrazol-3-yl)cyclopropanecarboxamide;
N-[1-(2-fluoro-4-pyridyl)pyrazol-3-yl]-2-(5-methylpyrazol-1-yl)acetamide;
N-[1-(2-fluoro-4-pyridyl)pyrazol-3-yl]-2-pyrazol-1-yl-acetamide;
N-[1-(2-fluoro-4-pyridyl)pyrazol-3-yl]-2-(3-methylpyrazol-1-yl)acetamide;
1-(2,2-difluoro-1,3-benzodioxol-5-yl)-N-[1-(2-fluoro-4-pyridyl)pyrazol-3-yl]cyclopropanecarboxamide;
N-[1-(2-fluoro-4-pyridyl)pyrazol-3-yl]-2-imidazol-1-yl-acetamide;
N-[1-(2-fluoro-4-pyridyl)pyrazol-3-yl]-2-(4-methylpyrazol-1-yl)acetamide;
2-(5-methylpyrazol-1-yl)-N-(1-phenylpyrazol-3-yl)acetamide;
N-(1-phenylpyrazol-3-yl)-2-pyrazol-1-yl-acetamide;
2-(4-methylpyrazol-1-yl)-N-(1-phenylpyrazol-3-yl)acetamide;
2-(3-methylpyrazol-1-yl)-N-(1-phenylpyrazol-3-yl)acetamide;
1-(2,2-difluoro-1,3-benzodioxol-5-yl)-N-(1-phenylpyrazol-3-yl)cyclopropanecarboxamide;
N-[1-(2-fluoro-4-pyridyl)pyrazol-3-yl]-2-(3-thienyl)acetamide;
N-[1-(2-fluoro-4-pyridyl)pyrazol-3-yl]-2-methyl-2-phenyl-butanamide;
N-[1-(2-fluoro-4-pyridyl)pyrazol-3-yl]-2-pyrrol-1-yl-acetamide;
2-(2-fluorophenyl)-2-methyl-N-(1-phenylpyrazol-3-yl)propanamide;
N-(1-phenylpyrazol-3-yl)-2-pyrrol-1-yl-acetamide;
N-(1-phenylpyrazol-3-yl)-2-(triazol-2-yl)acetamide;
N-[1-(2-fluoro-4-pyridyl)pyrazol-3-yl]-2-(triazol-2-yl)acetamide;
2-methyl-2-phenyl-N-(1-phenylpyrazol-3-yl)butanamide;
N-[1-(3,5-difluorophenyl)pyrazol-3-yl]-1-(2-fluorophenyl)cyclopropanecarboxamide;
N-[1-(2-fluoro-4-pyridyl)pyrazol-3-yl]-2-(1-methylimidazol-2-yl)acetamide;
N-[1-(2-fluoro-4-pyridyl)pyrazol-3-yl]-2-(1,2,4-triazol-1-yl)acetamide;
N-[1-(2-fluoro-4-pyridyl)pyrazol-3-yl]-2-isoxazol-3-yl-acetamide;
N-(1-phenylpyrazol-3-yl)-2-(1,2,4-triazol-1-yl)acetamide;
2-isoxazol-3-yl-N-(1-phenylpyrazol-3-yl)acetamide;
N-[1-(2-fluoro-4-pyridyl)pyrazol-3-yl]-2-thiazol-2-yl-acetamide;
N-[1-(3,5-difluorophenyl)pyrazol-3-yl]-1-phenyl-cyclopropanecarboxamide;
N-[1-(2-fluoro-4-pyridyl)pyrazol-3-yl]-2-(1-methylpyrrol-2-yl)acetamide;
N-(1-phenylpyrazol-3-yl)-2-thiazol-2-yl-acetamide;
2-(1-methylimidazol-2-yl)-N-(1-phenylpyrazol-3-yl)acetamide;
2-(1-methylpyrrol-2-yl)-N-(1-phenylpyrazol-3-yl)acetamide;
1-(3-fluoro-4-pyridyl)-N-[1-(2-fluoro-4-pyridyl)pyrazol-3-yl]cyclopropanecarboxamide;
1-(2-fluoro-5-methoxy-phenyl)-N-[1-(2-fluoro-4-pyridyl)pyrazol-3-yl]cyclopropanecarboxamide;
1-(2-fluorophenyl)-N-[1-[6-(trifluoromethyl)-3-pyridyl]pyrazol-3-yl]cyclopropanecarboxamide;
1-(2-fluorophenyl)-N-[1-[5-(trifluoromethyl)-3-pyridyl]pyrazol-3-yl]cyclopropanecarboxamide;
1-(2-fluorophenyl)-N-[1-(2-methoxy-3-pyridyl)pyrazol-3-yl]cyclopropanecarboxamide;
N-[1-(5-cyano-3-pyridyl) pyrazol-3-yl]-1-(2-fluorophenyl)cyclopropanecarboxamide;
1-(2-fluorophenyl)-N-[1-(6-methoxy-3-pyridyl)pyrazol-3-yl]cyclopropanecarboxamide;
N-[1-(2-cyano-3-pyridyl) pyrazol-3-yl]-1-(2-fluorophenyl)cyclopropanecarboxamide;
1-(2-fluorophenyl)-N-[1-(3,4,5-trimethoxyphenyl)pyrazol-3-yl]cyclopropanecarboxamide;
N-[1-(3-fluoro-4-methoxy-phenyl)pyrazol-3-yl]-1-(2-fluorophenyl)cyclopropanecarboxamide;
1-(2-fluorophenyl)-N-[1-(5-methyl-3-pyridyl)pyrazol-3-yl]cyclopropanecarboxamide;
1-(2-fluorophenyl)-N-[1-[2-(trifluoromethyl)-4-pyridyl]pyrazol-3-yl]cyclopropanecarboxamide;
N-[1-(5-chloro-3-pyridyl)pyrazol-3-yl]-1-(2-fluorophenyl)cyclopropanecarboxamide;
N-[1-(6-cyano-3-pyridyl) pyrazol-3-yl]-1-(2-fluorophenyl)cyclopropanecarboxamide;
N-[1-(3-cyano-5-methoxy-4-pyridyl)pyrazol-3-yl]-1-(2-fluorophenyl)cyclopropanecarboxamide;
1-(2-fluorophenyl)-N-[1-[3-(trifluoromethyl)-4-pyridyl]pyrazol-3-yl]cyclopropanecarboxamide;
N-[1-(2-cyano-4-pyridyl) pyrazol-3-yl]-1-(2-fluorophenyl)cyclopropanecarboxamide;
1-(2-fluorophenyl)-N-[1-(2-methoxypyrimidin-4-yl)pyrazol-3-yl]cyclopropanecarboxamide;
1-(2-fluorophenyl)-N-[1-(2-methylpyrimidin-4-yl)pyrazol-3-yl]cyclopropanecarboxamide;
1-(2-fluorophenyl)-N-[1-[2-(2,2,2-trifluoro-1-hydroxy-ethyl)-4-pyridyl]pyrazol-3-yl]cyclopropanecarboxamide;
N-[1-(3-chloro-4-pyridyl)pyrazol-3-yl]-1-(2-fluorophenyl)cyclopropanecarboxamide;
1-(2-fluorophenyl)-N-[1-[2-(1-hydroxy-1-methyl-ethyl)-4-pyridyl]pyrazol-3-yl]cyclopropanecarboxamide;
N-[1-(3-cyano-4-pyridyl) pyrazol-3-yl]-1-(2-fluorophenyl)cyclopropanecarboxamide;
N-[1-(3-cyano-2-methoxy-4-pyridyl)pyrazol-3-yl]-1-(2-fluorophenyl)cyclopropanecarboxamide;
N-[1-(2,6-di methyl pyrimidin-4-yl)pyrazol-3-yl]-1-(2-fluorophenyl)cyclopropanecarboxamide;
1-(2-fluorophenyl)-N-[1-(2-methoxypyrimidin-5-yl)pyrazol-3-yl]cyclopropanecarboxamide;
N-[1-(4,6-di methyl pyrimidin-5-yl)pyrazol-3-yl]-1-(2-fluorophenyl)cyclopropanecarboxamide;
1-(2-fluorophenyl)-N-[1-(2-methoxy-3-methyl-4-pyridyl)pyrazol-3-yl]cyclopropanecarboxamide;
1-(2-fluorophenyl)-N-[1-(4-methylpyrimidin-5-yl)pyrazol-3-yl]cyclopropanecarboxamide;
1-(2-fluorophenyl)-N-[1-(2-isopropoxy-4-pyridyl)pyrazol-3-yl]cyclopropanecarboxamide;
N-[1-(2,3-di methyl-4-pyridyl)pyrazol-3-yl]-1-(2-fluorophenyl)cyclopropanecarboxamide;
N-[1-(5-cyano-2-methyl-pyrimidin-4-yl)pyrazol-3-yl]-1-(2-fluorophenyl)cyclopropanecarboxamide;
N-[1-(3-fluoro-5-methoxy-4-pyridyl)pyrazol-3-yl]-1-(2-fluorophenyl)cyclopropanecarboxamide;
N-[1-(2-cyano-6-methyl-4-pyridyl)pyrazol-3-yl]-1-(2-fluorophenyl)cyclopropanecarboxamide;
N-[1-(3,5-di methoxy-4-pyridyl)pyrazol-3-yl]-1-(2-fluorophenyl)cyclopropanecarboxamide;
N-[1-(5-fluoro-6-methoxy-3-pyridyl)pyrazol-3-yl]-1-(2-fluorophenyl)cyclopropanecarboxamide;
N-[1-(5-fluoro-6-methyl-3-pyridyl)pyrazol-3-yl]-1-(2-fluorophenyl)cyclopropanecarboxamide;
N-[1-(2,5-di methyl-4-pyridyl)pyrazol-3-yl]-1-(2-fluorophenyl)cyclopropanecarboxamide;
N-[1-(2-fluoro-3-methyl-4-pyridyl)pyrazol-3-yl]-1-(2-fluorophenyl)cyclopropanecarboxamide;
N-[1-(3-cyano-5-fluoro-phenyl)pyrazol-3-yl]-1-(2-fluorophenyl)cyclopropanecarboxamide;
1-(2-fluorophenyl)-N-[1-(4-methoxypyrimidin-5-yl)pyrazol-3-yl]cyclopropanecarboxamide;
N-[1-[5-fluoro-6-(1-hydroxy-1-methyl-ethyl)-3-pyridyl]pyrazol-3-yl]-1-(2-fluorophenyl)cyclopropanecarboxamide;
N-[1-(3-fluoro-4-hydroxy-phenyl)pyrazol-3-yl]-1-(2-fluorophenyl)cyclopropanecarboxamide;
1-(2-fluorophenyl)-N-[1-(6-hydroxypyridazin-4-yl)pyrazol-3-yl]cyclopropanecarboxamide;
N-[1-(3,5-difluoro-4-hydroxy-phenyl)pyrazol-3-yl]-1-(2-fluorophenyl)cyclopropanecarboxamide;
1-(2-fluorophenyl)-N-[1-(6-methoxypyrimidin-4-yl)pyrazol-3-yl]cyclopropanecarboxamide;
1-(2-fluorophenyl)-N-[1-(6-methoxy-2-methyl-pyrimidin-4-yl)pyrazol-3-yl]cyclopropanecarboxamide;
1-(2-fluorophenyl)-N-[1-(5-methylpyrimidin-4-yl)pyrazol-3-yl]cyclopropanecarboxamide;
1-(2-fluorophenyl)-N-[1-(5-fluoropyrimidin-4-yl)pyrazol-3-yl]cyclopropanecarboxamide;
2-(2-fluorophenyl)-N-methyl-N-(1-phenylpyrazol-3-yl)acetamide;
1-(2-fluorophenyl)-N-[1-(2-fluoro-4-pyridyl)pyrazol-3-yl]-N-methyl-cyclopropanecarboxamide;
1-(2-fluorophenyl)-N-[1-(2-hydroxy-4-pyridyl)pyrazol-3-yl]cyclopropanecarboxamide;
N-[4-fluoro-1-(2-fluoro-4-pyridyl)pyrazol-3-yl]-1-(2-fluorophenyl)cyclopropanecarboxamide;
Rel-(R)-2,2-difluoro-1-phenyl-N-(1-phenyl-1H-pyrazol-3-yl)cyclopropane-1-carboxamide;
Rel-(S)-2,2-difluoro-1-phenyl-N-(1-phenyl-1H-pyrazol-3-yl)cyclopropane-1-carboxamide;
Rel-(R)-2,2-difluoro-N-(1-(2-fluoropyridin-4-yl)-1H-pyrazol-3-yl)-1-phenylcyclopropane-1-carboxamide;
Rel-(S)-2,2-difluoro-N-(1-(2-fluoropyridin-4-yl)-1H-pyrazol-3-yl)-1-phenylcyclopropane-1-carboxamide;
Rel-(R)-2,2-dichloro-N-(1-(2-fluoropyridin-4-yl)-1H-pyrazol-3-yl)-1-phenylcyclopropane-1-carboxamide;
Rel-(S)-2,2-dichloro-N-(1-(2-fluoropyridin-4-yl)-1H-pyrazol-3-yl)-1-phenylcyclopropane-1-carboxamide;
1-(2-fluorophenyl)-N-[1-(4-methylthiazol-2-yl)pyrazol-3-yl]cyclopropanecarboxamide;
1-(2-fluorophenyl)-N-(1-oxazol-2-ylpyrazol-3-yl)cyclopropanecarboxamide;
N-[1-(6-fluoro-5-methyl-3-pyridyl)pyrazol-3-yl]-1-(2-fluorophenyl)cyclopropanecarboxamide;
1-(2-fluorophenyl)-N-[1-(6-fluoro-3-pyridyl)pyrazol-3-yl]cyclopropanecarboxamide;
N-[1-(5-chloro-6-methoxy-3-pyridyl)pyrazol-3-yl]-1-(2-fluorophenyl)cyclopropanecarboxamide;
N-[1-(5,6-dichloro-3-pyridyl)pyrazol-3-yl]-1-(2-fluorophenyl)cyclopropanecarboxamide;
N-[1-(5-bromopyrimidin-2-yl)pyrazol-3-yl]-1-(2-fluorophenyl)cyclopropanecarboxamide;
1-(2-fluorophenyl)-N-[1-[2-(trifluoromethyl)pyrimidin-5-yl]pyrazol-3-yl]cyclopropanecarboxamide;
1-(2-fluorophenyl)-N-[1-[2-(1-hydroxy-1-methyl-ethyl)pyrimidin-5-yl]pyrazol-3-yl]cyclopropanecarboxamide;
N-[1-(2-tert-butyl pyrimidin-5-yl)pyrazol-3-yl]-1-(2-fluorophenyl)cyclopropanecarboxamide;
N-[1-(4-amino-3,5-difluoro-phenyl)pyrazol-3-yl]-1-(2-fluorophenyl)cyclopropanecarboxamide;
N-[1-(3-amino-4-fluoro-phenyl)pyrazol-3-yl]-1-(2-fluorophenyl)cyclopropanecarboxamide;
1-(2-fluorophenyl)-N-(5-methyl-1-phenyl-pyrazol-3-yl)cyclopropanecarboxamide;
N-[1-(4-amino-3-fluoro-phenyl)pyrazol-3-yl]-1-(2-fluorophenyl)cyclopropanecarboxamide;
1-(2-fluorophenyl)-N-[1-(thiadiazol-5-yl)pyrazol-3-yl]cyclopropanecarboxamide;
N-[1-(2-fluoro-4-pyridyl)pyrazol-3-yl]-1-(2-methoxyphenyl)cyclopropanecarboxamide;
1-(2-fluorophenyl)-N-[1-(1-methylpyrazol-4-yl)pyrazol-3-yl]cyclopropanecarboxamide;
N-[1-(3,5-difluoro-2-pyridyl)pyrazol-3-yl]-1-(2-fluorophenyl)cyclopropanecarboxamide;
1-(2-fluorophenyl)-N-[1-(1-methylimidazol-4-yl)pyrazol-3-yl]cyclopropanecarboxamide;
N-[1-(2-amino-4-pyridyl)pyrazol-3-yl]-1-(2-fluorophenyl)cyclopropanecarboxamide;
N-[1-[6-(dimethylamino)-3-pyridyl]pyrazol-3-yl]-1-(2-fluorophenyl)cyclopropanecarboxamide;
N-[1-[2-(difluoromethoxy)-4-pyridyl]pyrazol-3-yl]-1-(2-fluorophenyl)cyclopropanecarboxamide;
N-[1-[2-(difluoromethyl)-4-pyridyl]pyrazol-3-yl]-1-(2-fluorophenyl)cyclopropanecarboxamide;
N-[1-[6-(difluoromethyl)-3-pyridyl]pyrazol-3-yl]-1-(2-fluorophenyl)cyclopropanecarboxamide;
N-[1-(5-chloro-2-pyridyl)pyrazol-3-yl]-1-(2-fluorophenyl)cyclopropanecarboxamide;
N-[1-(6-amino-5-fluoro-3-pyridyl)pyrazol-3-yl]-1-(2-fluorophenyl)cyclopropanecarboxamide;
1-(2-fluorophenyl)-N-[1-(1-methyltriazol-4-yl)pyrazol-3-yl]cyclopropanecarboxamide;
1-(2-fluorophenyl)-N-[1-(3-methylisothiazol-5-yl)pyrazol-3-yl]cyclopropanecarboxamide;
1-(2-fluorophenyl)-N-(1-isothiazol-3-ylpyrazol-3-yl)cyclopropanecarboxamide;
N-[1-(1,3-di methylpyrazol-4-yl) pyrazol-3-yl]-1-(2-fluorophenyl)cyclopropanecarboxamide;
1-(2-fluorophenyl)-N-[1-(5-methyl-1,3,4-thiadiazol-2-yl)pyrazol-3-yl]cyclopropanecarboxamide;
N-[1-(1-ethylpyrazol-4-yl)pyrazol-3-yl]-1-(2-fluorophenyl)cyclopropanecarboxamide;
1-(2-fluorophenyl)-N-[1-(1,2,4-thiadiazol-5-yl)pyrazol-3-yl]cyclopropanecarboxamide;
N-[1-(2-methoxy-4-pyridyl)pyrazol-3-yl]-1-pyrimidin-2-yl-cyclopropanecarboxamide;
1-(2-fluorophenyl)-N-[1-(2-methoxythiazol-5-yl)pyrazol-3-yl]cyclopropanecarboxamide;
N-[1-[6-(difluoromethoxy)-3-pyridyl]pyrazol-3-yl]-1-(2-fluorophenyl)cyclopropanecarboxamide;
N-[1-(2-chlorothiazol-5-yl)pyrazol-3-yl]-1-(2-fluorophenyl)cyclopropanecarboxamide;
1-(2-fluorophenyl)-N-[1-(3-methoxy-4-pyridyl)pyrazol-3-yl]cyclopropanecarboxamide;
1-(2-fluorophenyl)-N-[1-[6-(methylamino)-3-pyridyl]pyrazol-3-yl]cyclopropanecarboxamide;
N-[1-(2,4-di methylthiazol-5-yl)pyrazol-3-yl]-1-(2-fluorophenyl)cyclopropanecarboxamide;
N-[1-[1-(difluoromethyl)-3-methyl-pyrazol-4-yl]pyrazol-3-yl]-1-(2-fluorophenyl)cyclopropanecarboxamide;
1-(2-fluorophenyl)-N-[1-(2-methyl-1,2,4-triazol-3-yl)pyrazol-3-yl]cyclopropanecarboxamide;
1-(2-fluorophenyl)-N-(1-isoxazol-3-ylpyrazol-3-yl)cyclopropanecarboxamide;
1-(2-fluorophenyl)-N-[1-(2-methylpyrazol-3-yl)pyrazol-3-yl]cyclopropanecarboxamide;
N-[1-(3-chlorophenyl)pyrazol-3-yl]-1-(3-fluoro-2-pyridyl)cyclopropanecarboxamide;
2,2-difluoro-1-phenyl-N-[1-(4-pyridyl)pyrazol-3-yl]cyclopropanecarboxamide;
2,2-difluoro-N-[1-(5-fluoro-3-pyridyl)pyrazol-3-yl]-1-phenyl-cyclopropanecarboxamide;
N-[1-(3-chlorophenyl)pyrazol-3-yl]-2,2-difluoro-1-phenyl-cyclopropanecarboxamide;
1-(3-fluoro-2-pyridyl)-N-[1-(4-pyridyl) pyrazol-3-yl]cyclopropanecarboxamide;
1-(3-fluoro-2-pyridyl)-N-[1-(5-fluoro-3-pyridyl)pyrazol-3-yl]cyclopropanecarboxamide;
1-(2-fluorophenyl)-N-(1-isoxazol-4-ylpyrazol-3-yl)cyclopropanecarboxamide;
1-(2-fluorophenyl)-N-[1-(1-methyl-1,2,4-triazol-3-yl)pyrazol-3-yl]cyclopropanecarboxamide;
1-(2-fluorophenyl)-N-[1-(3-methylimidazol-4-yl)pyrazol-3-yl]cyclopropanecarboxamide;
1-(2-fluorophenyl)-N-[1-(4-methyl-1,2,4-triazol-3-yl)pyrazol-3-yl]cyclopropanecarboxamide;
N-[1-(2,4-dimethoxyphenyl)pyrazol-3-yl]-1-(2-fluorophenyl)cyclopropanecarboxamide;
N-[1-(4-fluoro-2-methoxy-phenyl)pyrazol-3-yl]-1-(2-fluorophenyl)cyclopropanecarboxamide;
N-[1-(6-ethoxy-3-pyridyl)pyrazol-3-yl]-1-(2-fluorophenyl)cyclopropanecarboxamide;
N-[1-[5-fluoro-6-(methylamino)-3-pyridyl]pyrazol-3-yl]-1-(2-fluorophenyl)cyclopropanecarboxamide;
1-(2-fluorophenyl)-N-[1-[6-(trideuteriomethoxy)pyridazin-4-yl]pyrazol-3-yl]cyclopropanecarboxamide;
Rel-(S)-2,2-difluoro-1-phenyl-N-[1-(4-pyridyl)pyrazol-3-yl]cyclopropanecarboxamide;
Rel-(R)-2,2-difluoro-1-phenyl-N-[1-(4-pyridyl)pyrazol-3-yl]cyclopropanecarboxamide;
Rel-(S)-2,2-difluoro-N-[1-(5-fluoro-3-pyridyl)pyrazol-3-yl]-1-phenyl-cyclopropanecarboxamide;
Rel-(R)-2,2-difluoro-N-[1-(5-fluoro-3-pyridyl)pyrazol-3-yl]-1-phenyl-cyclopropanecarboxamide;
Rel-(S)—N-[1-(3-chlorophenyl)pyrazol-3-yl]-2,2-difluoro-1-phenyl-cyclopropanecarboxamide;
Rel-(R)—N-[1-(3-chlorophenyl)pyrazol-3-yl]-2,2-difluoro-1-phenyl-cyclopropanecarboxamide;
1-(2-fluorophenyl)-N-[1-[2-(trideuteriomethoxy)-4-pyridyl]pyrazol-3-yl]cyclopropanecarboxamide;
1-(2-fluorophenyl)-N-[1-[6-(trideuteriomethoxy)-3-pyridyl]pyrazol-3-yl]cyclopropanecarboxamide;
1-(2-fluorophenyl)-N-[1-[5-fluoro-6-(trideuteriomethoxy)-3-pyridyl]pyrazol-3-yl]cyclopropanecarboxamide;
N-[5-fluoro-1-(5-fluoro-6-methoxy-3-pyridyl)pyrazol-3-yl]-1-(2-fluorophenyl)cyclopropanecarboxamide;
1-(2-fluorophenyl)-N-[1-(5-methyl-1,3,4-oxadiazol-2-yl)pyrazol-3-yl]cyclopropanecarboxamide;
2-(hydroxymethyl)-1-phenyl-N-(1-phenylpyrazol-3-yl)cyclopropanecarboxamide;
N-[4-fluoro-1-(5-fluoro-3-pyridyl)pyrazol-3-yl]-1-phenyl-cyclopropanecarboxamide;
N-[1-(6-chloropyridazin-4-yl) pyrazol-3-yl]-1-(2-fluorophenyl)cyclopropanecarboxamide;
1-(2-fluorophenyl)-N-[1-(6-methoxypyridazin-4-yl)pyrazol-3-yl]cyclopropanecarboxamide;
N-[1-(2,4-difluorophenyl)pyrazol-3-yl]-1-(2-fluorophenyl)cyclopropanecarboxamide;
N-[1-(2-ethoxy-4-pyridyl)pyrazol-3-yl]-1-(2-fluorophenyl)cyclopropanecarboxamide;
N-[1-(4-fluoro-2-methyl-phenyl)pyrazol-3-yl]-1-(3-fluoro-2-pyridyl)cyclopropanecarboxamide;
N-[1-(2,4-difluorophenyl)-5-methyl-pyrazol-3-yl]-1-(3-fluoro-2-pyridyl)cyclopropanecarboxamide;
N-[1-(5-fluoro-6-methoxy-3-pyridyl)pyrazol-3-yl]-1-(3-fluoro-2-pyridyl)cyclopropanecarboxamide;
N-[1-(3-chloro-2-pyridyl)pyrazol-3-yl]-1-(3-fluoro-2-pyridyl)cyclopropanecarboxamide;
1-(3-fluoro-2-pyridyl)-N-[1-(2-methoxypyrimidin-5-yl)pyrazol-3-yl]cyclopropanecarboxamide;
N-[1-(5-chloro-3-pyridyl)pyrazol-3-yl]-1-(3-fluoro-2-pyridyl)cyclopropanecarboxamide;
N-[1-(4-fluorophenyl)pyrazol-3-yl]-1-(3-fluoro-2-pyridyl)cyclopropanecarboxamide;
1-(3-fluoro-2-pyridyl)-N-[1-[4-(trifluoromethyl)phenyl]pyrazol-3-yl]cyclopropanecarboxamide;
1-(3-fluoro-2-pyridyl)-N-(1-pyridazin-3-ylpyrazol-3-yl)cyclopropanecarboxamide;
1-(3-fluoro-2-pyridyl)-N-(1-pyrimidin-5-ylpyrazol-3-yl)cyclopropanecarboxamide;
1-(3-fluoro-2-pyridyl)-N-[1-(3-fluoro-2-pyridyl)pyrazol-3-yl]cyclopropanecarboxamide;
N-[1-(3,5-difluorophenyl)pyrazol-3-yl]-1-(3-fluoro-2-pyridyl)cyclopropanecarboxamide;
N-[5-fluoro-1-(5-fluoro-3-pyridyl)pyrazol-3-yl]-1-(3-fluoro-2-pyridyl)cyclopropanecarboxamide;
N-[5-fluoro-1-(5-fluoro-3-pyridyl)pyrazol-3-yl]-1-(2-fluorophenyl)cyclopropanecarboxamide;
N-[1-(5-fluoro-3-pyridyl)pyrazol-3-yl]-1-(2-pyridyl)cyclopropanecarboxamide;
N-[1-(5-fluoro-3-pyridyl)pyrazol-3-yl]-1-[3-(trifluoromethoxy)phenyl]cyclopropanecarboxamide;
1-(5-fluoro-2-pyridyl)-N-[1-(5-fluoro-3-pyridyl)pyrazol-3-yl]cyclopropanecarboxamide;
N-[1-(5-fluoro-2-methoxy-4-pyridyl)pyrazol-3-yl]-1-(2-fluorophenyl)cyclopropanecarboxamide;
N-[1-(4-fluoro-3-methoxy-phenyl)pyrazol-3-yl]-1-(2-fluorophenyl)cyclopropanecarboxamide;
1-(2-fluorophenyl)-N-[1-(4-methoxy-3-pyridyl)pyrazol-3-yl]cyclopropanecarboxamide;
N-[1-(2-fluoro-4-methoxy-phenylpyrazol-3-yl]-1-(2-fluorophenyl)cyclopropanecarboxamide;
1-(2-fluorophenyl)-N-[1-(2-methyl-3-pyridyl)pyrazol-3-yl]cyclopropanecarboxamide;
1-(2-fluorophenyl)-N-[1-(4-methyl-3-pyridyl)pyrazol-3-yl]cyclopropanecarboxamide;
2-(3-fluoro-2-pyridyl)-N-[1-(5-fluoro-3-pyridyl)pyrazol-3-yl]spiro[2.2]pentane-2-carboxamide;
N-(1-phenylpyrazol-3-yl)-1-(2-thienyl)cyclopropanecarboxamide;
1-(5-fluoro-2-pyridyl)-N-(1-phenylpyrazol-3-yl)cyclopropanecarboxamide;
2-methyl-N-(1-phenylpyrazol-3-yl)-2-(2-pyridyl)propanamide;
N-(1-phenylpyrazol-3-yl)-1-pyrazin-2-yl-cyclopropanecarboxamide;
N-[1-(2-methoxy-4-pyridyl)pyrazol-3-yl]-1-(2-thienyl)cyclopropanecarboxamide;
N-[1-(3,4-difluorophenyl)pyrazol-3-yl]-1-(2-thienyl)cyclopropanecarboxamide;
N-[1-(3,5-difluorophenyl)pyrazol-3-yl]-1-(2-thienyl)cyclopropanecarboxamide;
N-[1-(4-fluorophenyl)pyrazol-3-yl]-1-(2-thienyl)cyclopropanecarboxamide;
N-[1-(4-pyridyl)pyrazol-3-yl]-1-(2-thienyl)cyclopropanecarboxamide;
N-[1-(5-fluoro-3-pyridyl)pyrazol-3-yl]-1-(2-thienyl)cyclopropanecarboxamide;
N-[1-[5-(difluoromethoxy)-3-pyridyl]pyrazol-3-yl]-1-(2-thienyl)cyclopropanecarboxamide;
1-(5-fluoro-2-pyridyl)-N-[1-(2-methoxy-4-pyridyl)pyrazol-3-yl]cyclopropanecarboxamide;
N-[1-(3,4-difluorophenyl)pyrazol-3-yl]-1-(5-fluoro-2-pyridyl)cyclopropanecarboxamide;
N-[1-(3,5-difluorophenyl)pyrazol-3-yl]-1-(5-fluoro-2-pyridyl)cyclopropanecarboxamide;
N-[1-(4-fluorophenyl)pyrazol-3-yl]-1-(5-fluoro-2-pyridyl)cyclopropanecarboxamide;
1-(5-fluoro-2-pyridyl)-N-[1-(4-pyridyl) pyrazol-3-yl]cyclopropanecarboxamide;
N-[1-[5-(difluoromethoxy)-3-pyridyl]pyrazol-3-yl]-1-(5-fluoro-2-pyridyl)cyclopropanecarboxamide;
N-[1-(2-methoxy-4-pyridyl)pyrazol-3-yl]-1-pyrazin-2-yl-cyclopropanecarboxamide;
N-[1-(3,4-difluorophenyl)pyrazol-3-yl]-1-pyrazin-2-yl-cyclopropanecarboxamide;
N-[1-(3,5-difluorophenyl)pyrazol-3-yl]-1-pyrazin-2-yl-cyclopropanecarboxamide;
N-[1-(4-fluorophenyl)pyrazol-3-yl]-1-pyrazin-2-yl-cyclopropanecarboxamide;
1-pyrazin-2-yl-N-[1-(4-pyridyl)pyrazol-3-yl]cyclopropanecarboxamide;
N-[1-[5-(difluoromethoxy)-3-pyridyl]pyrazol-3-yl]-1-pyrazin-2-yl-cyclopropanecarboxamide;
1-(3-fluoro-5-methyl-2-pyridyl)-N-[1-(5-fluoro-3-pyridyl)pyrazol-3-yl]cyclopropanecarboxamide;
1-(5-chloro-3-fluoro-2-pyridyl)-N-[1-(5-fluoro-3-pyridyl)pyrazol-3-yl]cyclopropanecarboxamide;
Rel-(S)-2-(3-fluoro-2-pyridyl)-N-[1-(5-fluoro-3-pyridyl)pyrazol-3-yl]spiro[2.2]pentane-2-carboxamide;
Rel-(R)-2-(3-fluoro-2-pyridyl)-N-[1-(5-fluoro-3-pyridyl)pyrazol-3-yl]spiro[2.2]pentane-2-carboxamide;
1-(2-fluorophenyl)-N-[1-(4-methoxy-2-pyridyl)pyrazol-3-yl]cyclopropanecarboxamide;
1-(2-fluorophenyl)-N-[1-(6-methoxy-2-pyridyl)pyrazol-3-yl]cyclopropanecarboxamide;
1-(3-fluoro-2-pyridyl)-N-[1-(5-fluoro-3-pyridyl)pyrazol-3-yl]-2-methyl-cyclopropanecarboxamide;
N-[1-(5-chloro-3-pyridyl)pyrazol-3-yl]-1-(3-fluoro-2-pyridyl)-2-methyl-cyclopropanecarboxamide;
N-[1-(4-fluorophenyl)pyrazol-3-yl]-1-(3-fluoro-2-pyridyl)-2-methyl-cyclopropanecarboxamide;
N-[1-(3,4-difluorophenyl)pyrazol-3-yl]-1-(3-fluoro-2-pyridyl)-2-methyl-cyclopropanecarboxamide;
N-[1-(2,4-difluorophenyl)-5-methyl-pyrazol-3-yl]-1-(3-fluoro-2-pyridyl)-2-methyl-cyclopropanecarboxamide;
N-[1-(3,5-difluorophenyl)pyrazol-3-yl]-1-(3-fluoro-2-pyridyl)-2-methyl-cyclopropanecarboxamide;
N-[1-(5-fluoro-6-methoxy-3-pyridyl)pyrazol-3-yl]-1-(3-fluoro-2-pyridyl)-2-methyl-cyclopropanecarboxamide;
1-(3-fluoro-2-pyridyl)-N-[1-(2-methoxy-4-pyridyl)pyrazol-3-yl]-2-methyl-cyclopropanecarboxamide;
1-(3-fluoro-2-pyridyl)-N-[1-(5-fluoro-3-pyridyl)pyrazol-3-yl]-2,2-dimethyl-cyclopropanecarboxamide;
N-[1-(5-chloro-3-pyridyl)pyrazol-3-yl]-1-(3-fluoro-2-pyridyl)-2,2-dimethyl-cyclopropanecarboxamide;
N-[1-(4-fluorophenyl)pyrazol-3-yl]-1-(3-fluoro-2-pyridyl)-2,2-dimethyl-cyclopropanecarboxamide;
N-[1-(3,4-difluorophenyl)pyrazol-3-yl]-1-(3-fluoro-2-pyridyl)-2,2-di methyl-cyclopropanecarboxamide;
N-[1-(2,4-difluorophenyl)-5-methyl-pyrazol-3-yl]-1-(3-fluoro-2-pyridyl)-2,2-di methyl-cyclopropanecarboxamide;
N-[1-(3,5-difluorophenyl)pyrazol-3-yl]-1-(3-fluoro-2-pyridyl)-2,2-di methyl-cyclopropanecarboxamide;
N-[1-(5-fluoro-6-methoxy-3-pyridyl)pyrazol-3-yl]-1-(3-fluoro-2-pyridyl)-2,2-di methyl-cyclopropanecarboxamide;
1-(3-fluoro-2-pyridyl)-2,2-dimethyl-N-[1-(4-pyridyl)pyrazol-3-yl]cyclopropanecarboxamide;
1-(3-fluoro-2-pyridyl)-N-[1-(2-methoxy-4-pyridyl)pyrazol-3-yl]-2,2-di methyl-cyclopropanecarboxamide;
N-[1-(5-chloro-3-pyridyl)pyrazol-3-yl]-2-(3-fluoro-2-pyridyl)spiro[2.2]pentane-2-carboxamide;
N-[1-(4-fluorophenyl)pyrazol-3-yl]-2-(3-fluoro-2-pyridyl)spiro[2.2]pentane-2-carboxamide;
N-[1-(3,4-difluorophenyl)pyrazol-3-yl]-2-(3-fluoro-2-pyridyl)spiro[2.2]pentane-2-carboxamide;
N-[1-(2,4-difluorophenyl)-5-methyl-pyrazol-3-yl]-2-(3-fluoro-2-pyridyl)spiro[2.2]pentane-2-carboxamide;
N-[1-(3,5-difluorophenyl)pyrazol-3-yl]-2-(3-fluoro-2-pyridyl)spiro[2.2]pentane-2-carboxamide;
N-[1-(5-fluoro-6-methoxy-3-pyridyl)pyrazol-3-yl]-2-(3-fluoro-2-pyridyl)spiro[2.2]pentane-2-carboxamide;
2-(3-fluoro-2-pyridyl)-N-[1-(4-pyridyl) pyrazol-3-yl]spiro[2.2]pentane-2-carboxamide;
2-(3-fluoro-2-pyridyl)-N-[1-(2-methoxy-4-pyridyl)pyrazol-3-yl]spiro[2.2]pentane-2-carboxamide;
1-(3-fluoro-2-pyridyl)-2-methyl-N-[1-(4-pyridyl)pyrazol-3-yl]cyclopropanecarboxamide;
N-[1-(5-fluoro-3-pyridyl)pyrazol-3-yl]-1-pyrimidin-2-yl-cyclopropanecarboxamide;
N-[1-(5-chloro-3-pyridyl)pyrazol-3-yl]-1-pyrimidin-2-yl-cyclopropanecarboxamide;
N-[1-(4-fluorophenyl)pyrazol-3-yl]-1-pyrimidin-2-yl-cyclopropanecarboxamide;
N-[1-(3,4-difluorophenyl)pyrazol-3-yl]-1-pyrimidin-2-yl-cyclopropanecarboxamide;
N-[1-(2,4-difluorophenyl)-5-methyl-pyrazol-3-yl]-1-pyrimidin-2-yl-cyclopropanecarboxamide;
N-[1-(3,5-difluorophenyl)pyrazol-3-yl]-1-pyrimidin-2-yl-cyclopropanecarboxamide;
N-[1-(5-fluoro-6-methoxy-3-pyridyl)pyrazol-3-yl]-1-pyrimidin-2-yl-cyclopropanecarboxamide; and
N-[1-(4-pyridyl)pyrazol-3-yl]-1-pyrimidin-2-yl-cyclopropanecarboxamide;
or a pharmaceutically acceptable salt thereof.

13-24. (canceled)

25. A pharmaceutical composition comprising a chemical entity of claim 1 and a pharmaceutically acceptable carrier, adjuvant, or excipient.

26. A method of treating a disease, disorder or condition in a subject comprising administering to the subject an effective amount of the chemical entity of claim 1.

27. The method of claim 26, wherein the disease, disorder or condition is associated with (1) one or more mutations of ABCD1 transporter protein, (2) impaired peroxisomal beta-oxidation, (3) mutations of at least one of Acyl-CoA oxidase, D-Bifunctional protein, or ACBD5, or (4) accumulation of very long chain fatty acid (VLCFA) levels.

28. A method of treating ALD comprising administering to a subject an effective amount of a chemical entity of claim 1.

29. A method of reduction of very long chain fatty acids (VLCFA) levels in a subject comprising administering to the subject an effective amount of a chemical entity of claim 1.

30. A method of preparing the chemical entity of claim 1, comprising step (z): coupling a compound of formula: 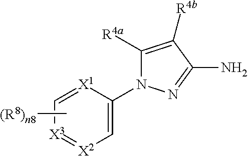
with a compound of formula: 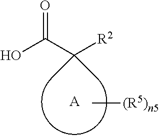
under conditions suitable to make the chemical entity.

31-33. (canceled)

34. The chemical entity of claim 12, wherein the chemical entity is a free compound selected from the group consisting of:
N-[1-(3-fluoro-2-pyridyl)pyrazol-3-yl]-1-phenyl-cyclopropanecarboxamide;
N-[1-(2-chloro-4-pyridyl)pyrazol-3-yl]-1-phenyl-cyclopropanecarboxamide;
1-(2-fluorophenyl)-N-[1-(2-fluoro-4-pyridyl)pyrazol-3-yl]cyclopropanecarboxamide;
1-(2-fluorophenyl)-N-[1-(3-fluoro-2-pyridyl)pyrazol-3-yl]cyclopropanecarboxamide;
1-phenyl-N-[1-(4-pyridyl)pyrazol-3-yl]cyclopropanecarboxamide;
1-(2-fluorophenyl)-N-[1-(4-pyridyl)pyrazol-3-yl]cyclopropanecarboxamide;
N-[1-(6-chloro-3-pyridyl)pyrazol-3-yl]-1-phenyl-cyclopropanecarboxamide;
N-[1-(6-chloro-3-pyridyl)pyrazol-3-yl]-1-(2-fluorophenyl)cyclopropanecarboxamide;
1-(4-fluorophenyl)-N-[1-(2-methyl-4-pyridyl)pyrazol-3-yl]cyclopropanecarboxamide;
N-[1-(2-methyl-4-pyridyl)pyrazol-3-yl]-1-phenyl-cyclopropanecarboxamide;
1-(2-fluorophenyl)-N-[1-(2-methyl-4-pyridyl)pyrazol-3-yl]cyclopropanecarboxamide;
N-[1-(2,5-difluoro-4-pyridyl)pyrazol-3-yl]-1-phenyl-cyclopropanecarboxamide;
N-[1-(2,5-difluoro-4-pyridyl)pyrazol-3-yl]-1-(2-fluorophenyl)cyclopropanecarboxamide;
N-[1-(6-methyl-3-pyridyl)pyrazol-3-yl]-1-phenyl-cyclopropanecarboxamide;
1-(2-fluorophenyl)-N-[1-(6-methyl-3-pyridyl)pyrazol-3-yl]cyclopropanecarboxamide;
1-(2-fluorophenyl)-N-[1-(3-fluoro-4-pyridyl)pyrazol-3-yl]cyclopropanecarboxamide;
N-[1-(3-fluoro-4-pyridyl)pyrazol-3-yl]-1-phenyl-cyclopropanecarboxamide;
2,2-dichloro-N-[1-(2-fluoro-4-pyridyl)pyrazol-3-yl]-1-phenyl-cyclopropanecarboxamide;
N-[1-(2-fluoro-4-pyridyl)pyrazol-3-yl]-3-phenyl-oxetane-3-carboxamide;
(2S)—N-[1-(2-fluoro-4-pyridyl)pyrazol-3-yl]-2-phenyl-spiro[2.4]heptane-2-carboxamide;
(2S)—N-[1-(2-fluoro-4-pyridyl)pyrazol-3-yl]-2-phenyl-spiro[2.3]hexane-2-carboxamide;
(2R)—N-[1-(2-fluoro-4-pyridyl)pyrazol-3-yl]-2-phenyl-spiro[2.4]heptane-2-carboxamide;
N-[1-(2-fluoro-4-pyridyl)pyrazol-3-yl]-2,2-dimethyl-1-phenyl-cyclopropanecarboxamide;
2-ethyl-N-[1-(2-fluoro-4-pyridyl)pyrazol-3-yl]-2-methyl-1-phenyl-cyclopropanecarboxamide;
2,2-difluoro-N-[1-(2-fluoro-4-pyridyl)pyrazol-3-yl]-1-phenyl-cyclopropanecarboxamide;
3,3-difluoro-N-[1-(2-fluoro-4-pyridyl)pyrazol-3-yl]-1-phenyl-cyclobutanecarboxamide;
1-(2-fluorophenyl)-N-[1-(5-fluoro-3-pyridyl)pyrazol-3-yl]cyclopropanecarboxamide;
N-[1-(5-fluoro-3-pyridyl)pyrazol-3-yl]-1-phenyl-cyclopropanecarboxamide;
1-(2-fluorophenyl)-N-[1-(2-pyridyl)pyrazol-3-yl]cyclopropanecarboxamide;
1-phenyl-N-[1-(2-pyridyl)pyrazol-3-yl]cyclopropanecarboxamide;
1-(2-fluorophenyl)-N-[1-(3-pyridyl)pyrazol-3-yl]cyclopropanecarboxamide;
N-[1-(2-fluoro-4-pyridyl)pyrazol-3-yl]-1-(3-pyridyl)cyclopropanecarboxamide;
N-[1-(2-fluoro-4-pyridyl)pyrazol-3-yl]-1-(2-pyridyl)cyclopropanecarboxamide;
1-(4-cyanophenyl)-N-[1-(2-fluoro-4-pyridyl)pyrazol-3-yl]cyclopropanecarboxamide;
1-phenyl-N-[1-(3-pyridyl)pyrazol-3-yl]cyclopropanecarboxamide;
N-[1-(2-fluoro-4-pyridyl)pyrazol-3-yl]-1-phenyl-cyclopropanecarboxamide;
N-[1-(2-fluoro-4-pyridyl)pyrazol-3-yl]-1-(4-iodophenyl)cyclopropanecarboxamide;
1-(3-cyanophenyl)-N-[1-(2-fluoro-4-pyridyl)pyrazol-3-yl]cyclopropanecarboxamide;
N-[1-(2-fluoro-4-pyridyl)pyrazol-3-yl]-1-(m-tolyl)cyclopropanecarboxamide;
1-(4-ethoxyphenyl)-N-[1-(2-fluoro-4-pyridyl)pyrazol-3-yl]cyclopropanecarboxamide;
1-(2-cyanophenyl)-N-[1-(2-fluoro-4-pyridyl)pyrazol-3-yl]cyclopropanecarboxamide;
1-(3,5-dimethylphenyl)-N-[1-(2-fluoro-4-pyridyl)pyrazol-3-yl]cyclopropanecarboxamide;
N-[1-(2-fluoro-4-pyridyl)pyrazol-3-yl]-1-(4-methoxyphenyl)cyclopropanecarboxamide;
N-[1-(2-fluoro-4-pyridyl)pyrazol-3-yl]-1-(3-methoxyphenyl)cyclopropanecarboxamide;
1-(2-chlorophenyl)-N-[1-(2-fluoro-4-pyridyl)pyrazol-3-yl]cyclopropanecarboxamide;
N-[1-(2-fluoro-4-pyridyl)pyrazol-3-yl]-1-(o-tolyl)cyclopropanecarboxamide;
1-(2,4-dichlorophenyl)-N-[1-(2-fluoro-4-pyridyl)pyrazol-3-yl]cyclopropanecarboxamide;
1-(3-fluorophenyl)-N-[1-(2-fluoro-4-pyridyl)pyrazol-3-yl]cyclopropanecarboxamide;
1-(4-fluorophenyl)-N-[1-(2-fluoro-4-pyridyl)pyrazol-3-yl]cyclopropanecarboxamide;
1-(3-chlorophenyl)-N-[1-(2-fluoro-4-pyridyl)pyrazol-3-yl]cyclopropanecarboxamide;
1-(4-chlorophenyl)-N-[1-(2-fluoro-4-pyridyl)pyrazol-3-yl]cyclopropanecarboxamide;
1-(2-fluorophenyl)-N-[1-(2-methoxy-4-pyridyl)pyrazol-3-yl]cyclopropanecarboxamide;
1-(2,5-difluorophenyl)-N-[1-(2-fluoro-4-pyridyl)pyrazol-3-yl]cyclopropanecarboxamide;
1-(4-chloro-2-fluoro-phenyl)-N-[1-(2-fluoro-4-pyridyl)pyrazol-3-yl]cyclopropanecarboxamide;
1-(5-chloro-2-fluoro-phenyl)-N-[1-(2-fluoro-4-pyridyl)pyrazol-3-yl]cyclopropanecarboxamide;
1-(2,6-difluorophenyl)-N-[1-(2-fluoro-4-pyridyl)pyrazol-3-yl]cyclopropanecarboxamide;
1-(2,3-difluorophenyl)-N-[1-(2-fluoro-4-pyridyl)pyrazol-3-yl]cyclopropanecarboxamide;
1-(3,5-difluorophenyl)-N-[1-(2-fluoro-4-pyridyl)pyrazol-3-yl]cyclopropanecarboxamide;
1-(2-chloro-6-fluoro-3-methyl-phenyl)-N-[1-(2-fluoro-4-pyridyl)pyrazol-3-yl]cyclopropanecarboxamide;
1-(2-chloro-6-fluoro-phenyl)-N-[1-(2-fluoro-4-pyridyl)pyrazol-3-yl]cyclopropanecarboxamide;
2,2,3,3-tetradeuterio-1-(2-fluorophenyl)-N-[1-(2-fluoro-4-pyridyl)pyrazol-3-yl]cyclopropanecarboxamide;
1-(3-fluoro-2-pyridyl)-N-[1-(2-fluoro-4-pyridyl)pyrazol-3-yl]cyclopropanecarboxamide;
1-(2,2-difluoro-1,3-benzodioxol-5-yl)-N-[1-(2-fluoro-4-pyridyl)pyrazol-3-yl]cyclopropanecarboxamide;
1-(3-fluoro-4-pyridyl)-N-[1-(2-fluoro-4-pyridyl)pyrazol-3-yl]cyclopropanecarboxamide;
1-(2-fluoro-5-methoxy-phenyl)-N-[1-(2-fluoro-4-pyridyl)pyrazol-3-yl]cyclopropanecarboxamide;
1-(2-fluorophenyl)-N-[1-[6-(trifluoromethyl)-3-pyridyl]pyrazol-3-yl]cyclopropanecarboxamide;
1-(2-fluorophenyl)-N-[1-[5-(trifluoromethyl)-3-pyridyl]pyrazol-3-yl]cyclopropanecarboxamide;
1-(2-fluorophenyl)-N-[1-(2-methoxy-3-pyridyl)pyrazol-3-yl]cyclopropanecarboxamide;
N-[1-(5-cyano-3-pyridyl) pyrazol-3-yl]-1-(2-fluorophenyl)cyclopropanecarboxamide;
1-(2-fluorophenyl)-N-[1-(6-methoxy-3-pyridyl)pyrazol-3-yl]cyclopropanecarboxamide;
N-[1-(2-cyano-3-pyridyl) pyrazol-3-yl]-1-(2-fluorophenyl)cyclopropanecarboxamide;
1-(2-fluorophenyl)-N-[1-(5-methyl-3-pyridyl)pyrazol-3-yl]cyclopropanecarboxamide;
1-(2-fluorophenyl)-N-[1-[2-(trifluoromethyl)-4-pyridyl]pyrazol-3-yl]cyclopropanecarboxamide;
N-[1-(5-chloro-3-pyridyl)pyrazol-3-yl]-1-(2-fluorophenyl)cyclopropanecarboxamide;
N-[1-(6-cyano-3-pyridyl) pyrazol-3-yl]-1-(2-fluorophenyl)cyclopropanecarboxamide;
N-[1-(3-cyano-5-methoxy-4-pyridyl)pyrazol-3-yl]-1-(2-fluorophenyl)cyclopropanecarboxamide;
1-(2-fluorophenyl)-N-[1-[3-(trifluoromethyl)-4-pyridyl]pyrazol-3-yl]cyclopropanecarboxamide;
N-[1-(2-cyano-4-pyridyl) pyrazol-3-yl]-1-(2-fluorophenyl)cyclopropanecarboxamide;
1-(2-fluorophenyl)-N-[1-[2-(2,2,2-trifluoro-1-hydroxy-ethyl)-4-pyridyl]pyrazol-3-yl]cyclopropanecarboxamide;
N-[1-(3-chloro-4-pyridyl)pyrazol-3-yl]-1-(2-fluorophenyl)cyclopropanecarboxamide;
1-(2-fluorophenyl)-N-[1-[2-(1-hydroxy-1-methyl-ethyl)-4-pyridyl]pyrazol-3-yl]cyclopropanecarboxamide;
N-[1-(3-cyano-4-pyridyl) pyrazol-3-yl]-1-(2-fluorophenyl)cyclopropanecarboxamide;
N-[1-(3-cyano-2-methoxy-4-pyridyl)pyrazol-3-yl]-1-(2-fluorophenyl)cyclopropanecarboxamide;
1-(2-fluorophenyl)-N-[1-(2-methoxy-3-methyl-4-pyridyl)pyrazol-3-yl]cyclopropanecarboxamide;
1-(2-fluorophenyl)-N-[1-(2-isopropoxy-4-pyridyl)pyrazol-3-yl]cyclopropanecarboxamide;
N-[1-(2,3-dimethyl-4-pyridyl)pyrazol-3-yl]-1-(2-fluorophenyl)cyclopropanecarboxamide;
N-[1-(3-fluoro-5-methoxy-4-pyridyl)pyrazol-3-yl]-1-(2-fluorophenyl)cyclopropanecarboxamide;
N-[1-(2-cyano-6-methyl-4-pyridyl)pyrazol-3-yl]-1-(2-fluorophenyl)cyclopropanecarboxamide;
N-[1-(3,5-dimethoxy-4-pyridyl)pyrazol-3-yl]-1-(2-fluorophenyl)cyclopropanecarboxamide;
N-[1-(5-fluoro-6-methoxy-3-pyridyl)pyrazol-3-yl]-1-(2-fluorophenyl)cyclopropanecarboxamide;
N-[1-(5-fluoro-6-methyl-3-pyridyl)pyrazol-3-yl]-1-(2-fluorophenyl)cyclopropanecarboxamide;
N-[1-(2,5-dimethyl-4-pyridyl)pyrazol-3-yl]-1-(2-fluorophenyl)cyclopropanecarboxamide;
N-[1-(2-fluoro-3-methyl-4-pyridyl)pyrazol-3-yl]-1-(2-fluorophenyl)cyclopropanecarboxamide;
N-[1-[5-fluoro-6-(1-hydroxy-1-methyl-ethyl)-3-pyridyl]pyrazol-3-yl]-1-(2-fluorophenyl)cyclopropanecarboxamide;
1-(2-fluorophenyl)-N-[1-(2-fluoro-4-pyridyl)pyrazol-3-yl]-N-methyl-cyclopropanecarboxamide;
1-(2-fluorophenyl)-N-[1-(2-hydroxy-4-pyridyl)pyrazol-3-yl]cyclopropanecarboxamide;
N-[4-fluoro-1-(2-fluoro-4-pyridyl)pyrazol-3-yl]-1-(2-fluorophenyl)cyclopropanecarboxamide;
Rel-(R)-2,2-difluoro-N-(1-(2-fluoropyridin-4-yl)-1H-pyrazol-3-yl)-1-phenylcyclopropane-1-carboxamide;
Rel-(S)-2,2-difluoro-N-(1-(2-fluoropyridin-4-yl)-1H-pyrazol-3-yl)-1-phenylcyclopropane-1-carboxamide;
Rel-(R)-2,2-dichloro-N-(1-(2-fluoropyridin-4-yl)-1H-pyrazol-3-yl)-1-phenylcyclopropane-1-carboxamide;
Rel-(S)-2,2-dichloro-N-(1-(2-fluoropyridin-4-yl)-1H-pyrazol-3-yl)-1-phenylcyclopropane-1-carboxamide;
N-[1-(6-fluoro-5-methyl-3-pyridyl)pyrazol-3-yl]-1-(2-fluorophenyl)cyclopropanecarboxamide;
1-(2-fluorophenyl)-N-[1-(6-fluoro-3-pyridyl)pyrazol-3-yl]cyclopropanecarboxamide;
N-[1-(5-chloro-6-methoxy-3-pyridyl)pyrazol-3-yl]-1-(2-fluorophenyl)cyclopropanecarboxamide;
N-[1-(5,6-dichloro-3-pyridyl)pyrazol-3-yl]-1-(2-fluorophenyl)cyclopropanecarboxamide;
N-[1-(2-fluoro-4-pyridyl)pyrazol-3-yl]-1-(2-methoxyphenyl)cyclopropanecarboxamide;
N-[1-(3,5-difluoro-2-pyridyl)pyrazol-3-yl]-1-(2-fluorophenyl)cyclopropanecarboxamide;
N-[1-(2-amino-4-pyridyl)pyrazol-3-yl]-1-(2-fluorophenyl)cyclopropanecarboxamide;
N-[1-[6-(dimethylamino)-3-pyridyl]pyrazol-3-yl]-1-(2-fluorophenyl)cyclopropanecarboxamide;
N-[1-[2-(difluoromethoxy)-4-pyridyl]pyrazol-3-yl]-1-(2-fluorophenyl)cyclopropanecarboxamide;
N-[1-[2-(difluoromethyl)-4-pyridyl]pyrazol-3-yl]-1-(2-fluorophenyl)cyclopropanecarboxamide;
N-[1-[6-(difluoromethyl)-3-pyridyl]pyrazol-3-yl]-1-(2-fluorophenyl)cyclopropanecarboxamide;
N-[1-(5-chloro-2-pyridyl)pyrazol-3-yl]-1-(2-fluorophenyl)cyclopropanecarboxamide;
N-[1-(6-amino-5-fluoro-3-pyridyl)pyrazol-3-yl]-1-(2-fluorophenyl)cyclopropanecarboxamide;
N-[1-(2-methoxy-4-pyridyl)pyrazol-3-yl]-1-pyrimidin-2-yl-cyclopropanecarboxamide;
N-[1-[6-(difluoromethoxy)-3-pyridyl]pyrazol-3-yl]-1-(2-fluorophenyl)cyclopropanecarboxamide;
1-(2-fluorophenyl)-N-[1-(3-methoxy-4-pyridyl)pyrazol-3-yl]cyclopropanecarboxamide;
1-(2-fluorophenyl)-N-[1-[6-(methylamino)-3-pyridyl]pyrazol-3-yl]cyclopropanecarboxamide;
2,2-difluoro-1-phenyl-N-[1-(4-pyridyl)pyrazol-3-yl]cyclopropanecarboxamide;
2,2-difluoro-N-[1-(5-fluoro-3-pyridyl)pyrazol-3-yl]-1-phenyl-cyclopropanecarboxamide;
1-(3-fluoro-2-pyridyl)-N-[1-(4-pyridyl)pyrazol-3-yl]cyclopropanecarboxamide;
1-(3-fluoro-2-pyridyl)-N-[1-(5-fluoro-3-pyridyl)pyrazol-3-yl]cyclopropanecarboxamide;
N-[1-(6-ethoxy-3-pyridyl)pyrazol-3-yl]-1-(2-fluorophenyl)cyclopropanecarboxamide;
N-[1-[5-fluoro-6-(methylamino)-3-pyridyl]pyrazol-3-yl]-1-(2-fluorophenyl)cyclopropanecarboxamide;
Rel-(S)-2,2-difluoro-1-phenyl-N-[1-(4-pyridyl)pyrazol-3-yl]cyclopropanecarboxamide;
Rel-(R)-2,2-difluoro-1-phenyl-N-[1-(4-pyridyl)pyrazol-3-yl]cyclopropanecarboxamide;
Rel-(S)-2,2-difluoro-N-[1-(5-fluoro-3-pyridyl)pyrazol-3-yl]-1-phenyl-cyclopropanecarboxamide;
Rel-(R)-2,2-difluoro-N-[1-(5-fluoro-3-pyridyl)pyrazol-3-yl]-1-phenyl-cyclopropanecarboxamide;
1-(2-fluorophenyl)-N-[1-[2-(trideuteriomethoxy)-4-pyridyl]pyrazol-3-yl]cyclopropanecarboxamide;
1-(2-fluorophenyl)-N-[1-[6-(trideuteriomethoxy)-3-pyridyl]pyrazol-3-yl]cyclopropanecarboxamide;
1-(2-fluorophenyl)-N-[1-[5-fluoro-6-(trideuteriomethoxy)-3-pyridyl]pyrazol-3-yl]cyclopropanecarboxamide;
N-[5-fluoro-1-(5-fluoro-6-methoxy-3-pyridyl)pyrazol-3-yl]-1-(2-fluorophenyl)cyclopropanecarboxamide;
N-[4-fluoro-1-(5-fluoro-3-pyridyl)pyrazol-3-yl]-1-phenyl-cyclopropanecarboxamide;
N-[1-(2-ethoxy-4-pyridyl)pyrazol-3-yl]-1-(2-fluorophenyl)cyclopropanecarboxamide;
N-[1-(5-fluoro-6-methoxy-3-pyridyl)pyrazol-3-yl]-1-(3-fluoro-2-pyridyl)cyclopropanecarboxamide;
N-[1-(3-chloro-2-pyridyl)pyrazol-3-yl]-1-(3-fluoro-2-pyridyl)cyclopropanecarboxamide;
N-[1-(5-chloro-3-pyridyl)pyrazol-3-yl]-1-(3-fluoro-2-pyridyl)cyclopropanecarboxamide;
1-(3-fluoro-2-pyridyl)-N-[1-(3-fluoro-2-pyridyl)pyrazol-3-yl]cyclopropanecarboxamide;
N-[5-fluoro-1-(5-fluoro-3-pyridyl)pyrazol-3-yl]-1-(3-fluoro-2-pyridyl)cyclopropanecarboxamide;
N-[5-fluoro-1-(5-fluoro-3-pyridyl)pyrazol-3-yl]-1-(2-fluorophenyl)cyclopropanecarboxamide;
N-[1-(5-fluoro-3-pyridyl)pyrazol-3-yl]-1-(2-pyridyl)cyclopropanecarboxamide;
N-[1-(5-fluoro-3-pyridyl)pyrazol-3-yl]-1-[3-(trifluoromethoxy)phenyl]cyclopropanecarboxamide;
1-(5-fluoro-2-pyridyl)-N-[1-(5-fluoro-3-pyridyl)pyrazol-3-yl]cyclopropanecarboxamide;
N-[1-(5-fluoro-2-methoxy-4-pyridyl)pyrazol-3-yl]-1-(2-fluorophenyl)cyclopropanecarboxamide;
1-(2-fluorophenyl)-N-[1-(4-methoxy-3-pyridyl)pyrazol-3-yl]cyclopropanecarboxamide;
1-(2-fluorophenyl)-N-[1-(2-methyl-3-pyridyl)pyrazol-3-yl]cyclopropanecarboxamide;
1-(2-fluorophenyl)-N-[1-(4-methyl-3-pyridyl)pyrazol-3-yl]cyclopropanecarboxamide;
2-(3-fluoro-2-pyridyl)-N-[1-(5-fluoro-3-pyridyl)pyrazol-3-yl]spiro[2.2]pentane-2-carboxamide;
N-[1-(2-methoxy-4-pyridyl)pyrazol-3-yl]-1-(2-thienyl)cyclopropanecarboxamide;
N-[1-(4-pyridyl)pyrazol-3-yl]-1-(2-thienyl)cyclopropanecarboxamide;
N-[1-(5-fluoro-3-pyridyl)pyrazol-3-yl]-1-(2-thienyl)cyclopropanecarboxamide;
N-[1-[5-(difluoromethoxy)-3-pyridyl]pyrazol-3-yl]-1-(2-thienyl)cyclopropanecarboxamide;
1-(5-fluoro-2-pyridyl)-N-[1-(2-methoxy-4-pyridyl)pyrazol-3-yl]cyclopropanecarboxamide;
1-(5-fluoro-2-pyridyl)-N-[1-(4-pyridyl)pyrazol-3-yl]cyclopropanecarboxamide;
N-[1-[5-(difluoromethoxy)-3-pyridyl]pyrazol-3-yl]-1-(5-fluoro-2-pyridyl)cyclopropanecarboxamide;
N-[1-(2-methoxy-4-pyridyl)pyrazol-3-yl]-1-pyrazin-2-yl-cyclopropanecarboxamide;
1-pyrazin-2-yl-N-[1-(4-pyridyl)pyrazol-3-yl]cyclopropanecarboxamide;
N-[1-[5-(difluoromethoxy)-3-pyridyl]pyrazol-3-yl]-1-pyrazin-2-yl-cyclopropanecarboxamide;
1-(3-fluoro-5-methyl-2-pyridyl)-N-[1-(5-fluoro-3-pyridyl)pyrazol-3-yl]cyclopropanecarboxamide;
1-(5-chloro-3-fluoro-2-pyridyl)-N-[1-(5-fluoro-3-pyridyl)pyrazol-3-yl]cyclopropanecarboxamide;
Rel-(S)-2-(3-fluoro-2-pyridyl)-N-[1-(5-fluoro-3-pyridyl)pyrazol-3-yl]spiro[2.2]pentane-2-carboxamide;
Rel-(R)-2-(3-fluoro-2-pyridyl)-N-[1-(5-fluoro-3-pyridyl)pyrazol-3-yl]spiro[2.2]pentane-2-carboxamide;
1-(2-fluorophenyl)-N-[1-(4-methoxy-2-pyridyl)pyrazol-3-yl]cyclopropanecarboxamide;
1-(2-fluorophenyl)-N-[1-(6-methoxy-2-pyridyl)pyrazol-3-yl]cyclopropanecarboxamide;
1-(3-fluoro-2-pyridyl)-N-[1-(5-fluoro-3-pyridyl)pyrazol-3-yl]-2-methyl-cyclopropanecarboxamide;
N-[1-(5-chloro-3-pyridyl)pyrazol-3-yl]-1-(3-fluoro-2-pyridyl)-2-methyl-cyclopropanecarboxamide;
N-[1-(5-fluoro-6-methoxy-3-pyridyl)pyrazol-3-yl]-1-(3-fluoro-2-pyridyl)-2-methyl-cyclopropanecarboxamide;
1-(3-fluoro-2-pyridyl)-N-[1-(2-methoxy-4-pyridyl)pyrazol-3-yl]-2-methyl-cyclopropanecarboxamide;
1-(3-fluoro-2-pyridyl)-N-[1-(5-fluoro-3-pyridyl)pyrazol-3-yl]-2,2-dimethyl-cyclopropanecarboxamide;
N-[1-(5-chloro-3-pyridyl)pyrazol-3-yl]-1-(3-fluoro-2-pyridyl)-2,2-dimethyl-cyclopropanecarboxamide;
N-[1-(5-fluoro-6-methoxy-3-pyridyl)pyrazol-3-yl]-1-(3-fluoro-2-pyridyl)-2,2-di methyl-cyclopropanecarboxamide;
1-(3-fluoro-2-pyridyl)-2,2-dimethyl-N-[1-(4-pyridyl)pyrazol-3-yl]cyclopropanecarboxamide;
1-(3-fluoro-2-pyridyl)-N-[1-(2-methoxy-4-pyridyl)pyrazol-3-yl]-2,2-dimethyl-cyclopropanecarboxamide;
N-[1-(5-chloro-3-pyridyl)pyrazol-3-yl]-2-(3-fluoro-2-pyridyl)spiro[2.2]pentane-2-carboxamide;
N-[1-(5-fluoro-6-methoxy-3-pyridyl)pyrazol-3-yl]-2-(3-fluoro-2-pyridyl)spiro[2.2]pentane-2-carboxamide;
2-(3-fluoro-2-pyridyl)-N-[1-(4-pyridyl)pyrazol-3-yl]spiro[2.2]pentane-2-carboxamide;
2-(3-fluoro-2-pyridyl)-N-[1-(2-methoxy-4-pyridyl)pyrazol-3-yl]spiro[2.2]pentane-2-carboxamide;
1-(3-fluoro-2-pyridyl)-2-methyl-N-[1-(4-pyridyl)pyrazol-3-yl]cyclopropanecarboxamide;
N-[1-(5-fluoro-3-pyridyl)pyrazol-3-yl]-1-pyrimidin-2-yl-cyclopropanecarboxamide;
N-[1-(5-chloro-3-pyridyl)pyrazol-3-yl]-1-pyrimidin-2-yl-cyclopropanecarboxamide;
N-[1-(5-fluoro-6-methoxy-3-pyridyl)pyrazol-3-yl]-1-pyrimidin-2-yl-cyclopropanecarboxamide; and
N-[1-(4-pyridyl)pyrazol-3-yl]-1-pyrimidin-2-yl-cyclopropanecarboxamide;
or a pharmaceutically acceptable salt thereof.

35. The chemical entity of claim 12, wherein the chemical entity is a free compound selected from the group consisting of: 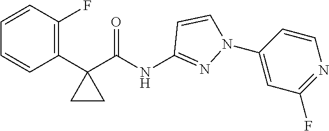 [SMILES: O=C(Nc1ccn(-c2ccnc(F)c2)n1)C1(c2ccccc2F)CC1]
1-(2-fluorophenyl)-N-[1-(2-fluoro-4-pyridyl)pyrazol-3-yl]cyclopropanecarboxamide; 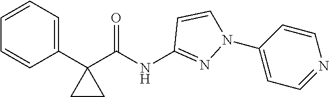 [SMILES: O=C(Nc1ccn(-c2ccncc2)n1)C1(c2ccccc2)CC1]
1-phenyl-N-[1-(4-pyridyl)pyrazol-3-yl]cyclopropanecarboxamide; 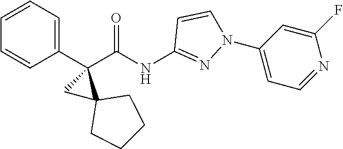 [SMILES: O=C(Nc1ccn(-c2ccnc(F)c2)n1)[C@@]1(c2ccccc2)CC12CCCC2]
(2S)—N-[1-(2-fluoro-4-pyridyl)pyrazol-3-yl]-2-phenyl-spiro[2.4]heptane-2-carboxamide; 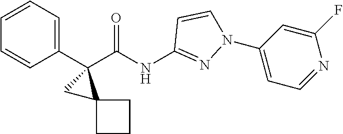 [SMILES: O=C(Nc1ccn(-c2ccnc(F)c2)n1)[C@@]1(c2ccccc2)CC12CCC2]
(2S)—N-[1-(2-fluoro-4-pyridyl)pyrazol-3-yl]-2-phenyl-spiro[2.3]hexane-2-carboxamide; 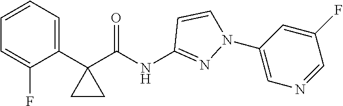 [SMILES: O=C(Nc1ccn(-c2cncc(F)c2)n1)C1(c2ccccc2F)CC1]
1-(2-fluorophenyl)-N-[1-(5-fluoro-3-pyridyl)pyrazol-3-yl]cyclopropanecarboxamide; 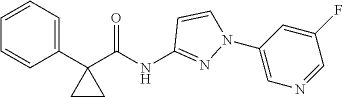 [SMILES: O=C(Nc1ccn(-c2cncc(F)c2)n1)C1(c2ccccc2)CC1]
N-[1-(5-fluoro-3-pyridyl)pyrazol-3-yl]-1-phenyl-cyclopropanecarboxamide; 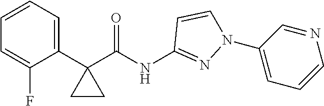 [SMILES: O=C(Nc1ccn(-c2cccnc2)n1)C1(c2ccccc2F)CC1]
1-(2-fluorophenyl)-N-[1-(3-pyridyl)pyrazol-3-yl]cyclopropanecarboxamide; 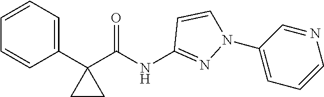 [SMILES: O=C(Nc1ccn(-c2cccnc2)n1)C1(c2ccccc2)CC1]
1-phenyl-N-[1-(3-pyridyl)pyrazol-3-yl]cyclopropanecarboxamide; 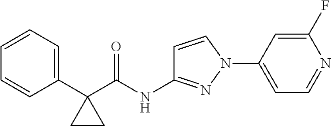 [SMILES: O=C(Nc1ccn(-c2ccnc(F)c2)n1)C1(c2ccccc2)CC1]
N-[1-(2-fluoro-4-pyridyl)pyrazol-3-yl]-1-phenyl-cyclopropanecarboxamide; 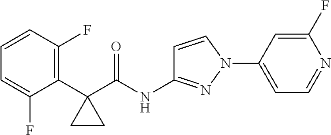 [SMILES: O=C(Nc1ccn(-c2ccnc(F)c2)n1)C1(c2c(F)cccc2F)CC1]
1-(2,6-difluorophenyl)-N-[1-(2-fluoro-4-pyridyl)pyrazol-3-yl]cyclopropanecarboxamide; 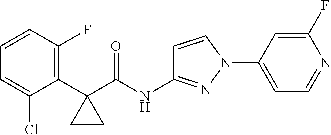 [SMILES: O=C(Nc1ccn(-c2ccnc(F)c2)n1)C1(c2c(F)cccc2Cl)CC1]
1-(2-chloro-6-fluoro-phenyl)-N-[1-(2-fluoro-4-pyridyl)pyrazol-3-yl]cyclopropanecarboxamide; 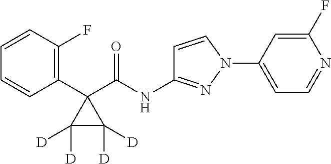 [SMILES: [2H]C1([2H])C([2H])([2H])C1(C(=O)Nc1ccn(-c2ccnc(F)c2)n1)c1ccccc1F]
2,2,3,3-tetradeuterio-1-(2-fluorophenyl)-N-[1-(2-fluoro-4-pyridyl)pyrazol-3-yl]cyclopropanecarboxamide; 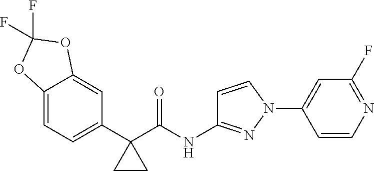 [SMILES: O=C(Nc1ccn(-c2ccnc(F)c2)n1)C1(c2ccc3c(c2)OC(F)(F)O3)CC1]
1-(2,2-difluoro-1,3-benzodioxol-5-yl)-N-[1-(2-fluoro-4-pyridyl)pyrazol-3-yl]cyclopropanecarboxamide; 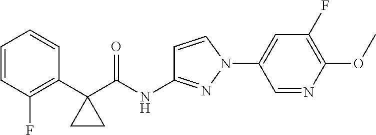 [SMILES: COc1ncc(-n2ccc(NC(=O)C3(c4ccccc4F)CC3)n2)cc1F]
N-[1-(5-fluoro-6-methoxy-3-pyridyl)pyrazol-3-yl]-1-(2-fluorophenyl)cyclopropanecarboxamide; 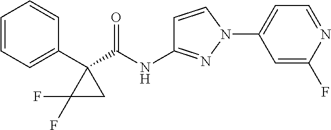 [SMILES: O=C(Nc1ccn(-c2ccnc(F)c2)n1)[C@]1(c2ccccc2)CC1(F)F]
Rel-(R)-2,2-difluoro-N-(1-(2-fluoropyridin-4-yl)-1H-pyrazol-3-yl)-1-phenylcyclopropane-1-carboxamide; 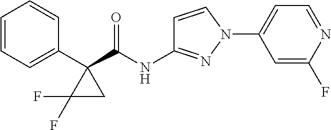 [SMILES: O=C(Nc1ccn(-c2ccnc(F)c2)n1)[C@@]1(c2ccccc2)CC1(F)F]
Rel-(S)-2,2-difluoro-N-(1-(2-fluoropyridin-4-yl)-1H-pyrazol-3-yl)-1-phenylcyclopropane-1-carboxamide; 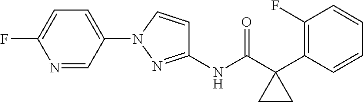 [SMILES: O=C(Nc1ccn(-c2ccc(F)nc2)n1)C1(c2ccccc2F)CC1]
1-(2-fluorophenyl)-N-[1-(6-fluoro-3-pyridyl)pyrazol-3-yl]cyclopropanecarboxamide; 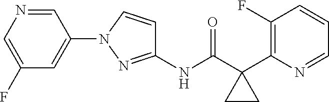 [SMILES: O=C(Nc1ccn(-c2cncc(F)c2)n1)C1(c2ncccc2F)CC1]
1-(3-fluoro-2-pyridyl)-N-[1-(5-fluoro-3-pyridyl)pyrazol-3-yl]cyclopropanecarboxamide; 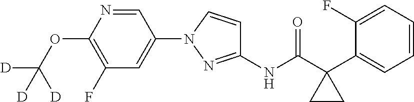 [SMILES: [2H]C([2H])([2H])Oc1ncc(-n2ccc(NC(=O)C3(c4ccccc4F)CC3)n2)cc1F]
1-(2-fluorophenyl)-N-[1-[5-fluoro-6-(trideuteriomethoxy)-3-pyridyl]pyrazol-3-yl]cyclopropanecarboxamide; 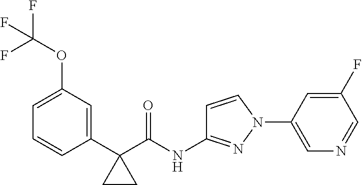 [SMILES: O=C(Nc1ccn(-c2cncc(F)c2)n1)C1(c2cccc(OC(F)(F)F)c2)CC1]
N-[1-(5-fluoro-3-pyridyl)pyrazol-3-yl]-1-[3-(trifluoromethoxy)phenyl]cyclopropanecarboxamide; 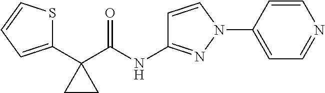 [SMILES: O=C(Nc1ccn(-c2ccncc2)n1)C1(c2cccs2)CC1]
N-[1-(4-pyridyl)pyrazol-3-yl]-1-(2-thienyl)cyclopropanecarboxamide; and 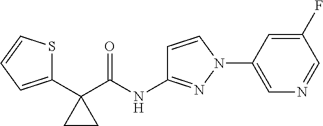 [SMILES: O=C(Nc1ccn(-c2cncc(F)c2)n1)C1(c2cccs2)CC1]
N-[1-(5-fluoro-3-pyridyl)pyrazol-3-yl]-1-(2-thienyl)cyclopropanecarboxamide;
or a pharmaceutically acceptable salt thereof.

36. The chemical entity of claim 35, wherein the chemical entity is a free compound.

37. The chemical entity of claim 35, wherein the chemical entity is a pharmaceutically acceptable salt.

38. The chemical entity of claim 1, which is a free compound of formula (II.B.1): 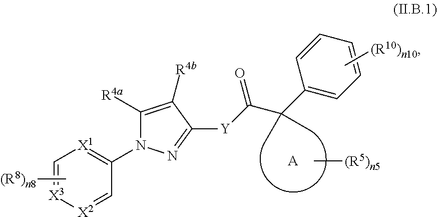
or a pharmaceutically acceptable salt thereof, wherein:
each instance of R¹⁰ independently is selected from halo, C₁₋₄ alkyl, C₁₋₄ haloalkyl, —(C(R^{J2a}₂))₀₋₂—OH, —(C(R^{J2a}₂))₀₋₂—OR^{J2}, —(C(R^{J2a}₂))₀₋₂—SR^{J2}, —(C(R^{J2a}₂))₀₋₂—NH₂, —(C(R^{J2a}₂))₀₋₂—NHR^{J2}, —(C(R^{J2a}₂))₀₋₂—NR^{J2}₂, —C(O)R^{J1}₂, and —CN,
wherein each instance of R^{J2} is independently C₁₋₃ alkyl or C₁₋₄ haloalkyl,
wherein each instance of R^{J2a} is independently H, C₁₋₃ alkyl, C₁₋₄ haloalkyl,
wherein optionally methylenedioxy constitutes a substituent of said phenyl, wherein the methylene unit of the methylenedioxy is unsubstituted or substituted with halo; and
n10 is 0, 1, 2 or 3.

39. The chemical entity of claim 1, wherein X¹ is N, and X² and X³ are CH.

40. The chemical entity of claim 1, wherein X² is N, and X¹ and X³ are CH.

41. The chemical entity of claim 1, wherein X³ is N, and X¹ and X² are CH.

42. The chemical entity of claim 1, wherein A is cyclopropane; and wherein each instance of R⁵ independently is C₁₋₄ alkyl or halo, or two geminal R⁵, together with the carbon atom to which they are attached, form a C₄₋₆ carbocycle.

43. The chemical entity of claim 1, wherein A is cyclopropane and n5 is 0.

44. The chemical entity of claim 38, wherein each instance of R¹⁰ independently is —F, —Cl, Me, Et, Pr, Bu, iPr, iBu, —OH, —OMe, —OEt, —OPr, —OiPr, NH₂, —NHMe, —NHEt, —NHiPr, —CF₃, —CHF₂ or —CN.

45. The chemical entity of claim 1, wherein Y is —NH—, and each of R^{4a} and R^{4b} is —H.

46. The chemical entity of claim 1, wherein each instance of R⁸ independently is halo, C₁₋₄ alkyl, C₁₋₄ haloalkyl, —OH, —OMe or —OEt.
